(19) United States
(12) Patent Application Publication
(10) Pub. No.: US 2024/0368122 A1
(43) Pub. Date: Nov. 7, 2024

(54) JAK2 INHIBITORS AND METHODS OF USE THEREOF

(71) Applicant: RELAY THERAPEUTICS, INC., Cambridge, MA (US)

(72) Inventors: Lucian V. DiPietro, Gloucester, MA (US); Ravi Kurukulasuriya, Cambridge, MA (US); Amael Madec, Cambridge, MA (US); Catherine A. Evans, Somerville, MA (US); Cary Griffin Fridrich, Cambridge, MA (US); Rebecca Jane Swett, Cambridge, MA (US); Alessandro Boezio, Cambridge, MA (US); Alexander M. Taylor, Cambridge, MA (US); Thomas H. McLean, Cambridge, MA (US); Christopher Thomson, Hoddesdon (GB)

(21) Appl. No.: 18/685,018

(22) PCT Filed: Aug. 19, 2022
(86) PCT No.: PCT/US2022/040835

§ 371 Date: Feb. 20, 2024

Related U.S. Application Data
(60) Provisional application No. 63/313,560, filed on Feb. 24, 2022.
(60) Provisional application No. 63/235,706, filed on Aug. 21, 2021.

(51) Int. Cl. C07D 401/14; A61K 31/501; A61K 31/502; A61K 31/506; A61K 31/675; C07D 237/24; C07D 401/12; C07D 403/12; C07D 405/14; C07D 409/14; C07D 413/14; C07D 417/12; C07D 417/14; C07D 471/04; C07D 487/04; C07F 9/6558

(52) U.S. Cl. CPC C07D 401/14; A61K 31/501; A61K 31/502; A61K 31/506; A61K 31/675; C07D 237/24; C07D 401/12; C07D 403/12; C07D 405/14; C07D 409/14; C07D 413/14; C07D 417/12; C07D 417/14; C07D 471/04; C07D 487/04; C07F 9/65583

(57) ABSTRACT

The present disclosure relates to novel compounds and pharmaceutical compositions thereof, and methods for inhibiting the activity of JAK2 enzymes with the compounds and compositions of the disclosure. The present disclosure further relates to, but is not limited to, methods for treating disorders associated with JAK2 signaling with the compounds and compositions of the disclosure.

### CROSS REFERENCE TO RELATED APPLICATIONS

This application claims the benefit of and priority to U.S. Provisional Patent Application No. 63/235,706, filed on Aug. 21, 2021; and U.S. Provisional Patent Application No. 63/313,560, filed Feb. 24, 2022, the content of each of which is hereby incorporated by reference in its entirety for all purposes.

### BACKGROUND

The Janus kinase (JAK) family of kinases (JAK1, JAK2, JAK3, and TYK2) are a family of intracellular, non-receptor tyrosine kinases that transduce cytokine-mediated signals. JAKs are in the cell selectively associated with the cytoplasmic domains of various cytokine receptors. Receptor-associated JAKs are activated in a ligand-dependent manner. Upon binding of the ligand and subsequent activation, JAKs can phosphorylate another JAK protein on the paired receptor and the intracellular tail of the receptors to which the JAKs are bound. These phosphorylated peptides serve as docking sites for a family of transcription factors, the signal transducers and activators of transcription (STAT). Upon binding of the STATs to the activated receptor-JAK complex, the STATs are phosphorylated, dimerize, and then are translocated to the nucleus where the binding of DNA and regulate gene expression occurs. Alterations in JAK2 signaling can occur through point mutations/deletions/insertions or chromosomal translocations. These JAK2 alterations drive diseases that are primarily characterized by abnormal proliferation of terminally differentiated myeloid cells. Examples of disease with JAK2 alterations are essential thrombocytosis or essential thrombocythemia (ET), polycythemia vera (PV), myelofibrosis (MF), primary myelofibrosis (PMF), and secondary myelofibrosis (SMF). Clinical features of these diseases include progressive anemia, splenomegaly, and constitutional symptoms (cough, fatigue, puritus, and bone pain).

Existing compounds that inhibit JAK2 (e.g. ruxoltinib and fedratinib) have been developed for these myeloproliferative indications and have proven beneficial to patients in terms of spleen volume reduction and symptomatic improvement. However, a significant limitation in the clinical effectiveness of existing JAK2 inhibitors has been the inability to achieve clinically effective doses while avoiding toxicity in patients with myeloid proliferative disease, due to their strong inhibition of wildtype JAK2. Treatment of patients with the approved JAK2 inhibitors can result in anemia and thrombocytopenia and these toxicities are consistent with the known function of wildtype JAK2 in regulation of erythrocytes and platelets. Thus, there exists a need for JAK2 compounds that inhibit mutant JAK2 activity while sparing cytokine-mediated activity of wildtype JAK2, in order to increase the therapeutic window and allow for more complete inhibition of the mutant protein.

### SUMMARY

In some embodiments, the present disclosure provides a compound of formula I:

Cy^{A}-L¹-Cy^{B} I

or a pharmaceutically acceptable salt thereof, wherein each of Cy^{A}, Cy^{B}, Cy^{C}, L¹, and L² is as defined in embodiments and classes and subclasses herein.

In some embodiments, the present disclosure provides a compound of formula I′:

Cy^{A}-L¹-Cy^{B} I′

or a pharmaceutically acceptable salt thereof, wherein each of Cy^{A}, Cy^{B}, Cy^{C}, L¹, and L² is as defined in embodiments and classes and subclasses herein.

In some embodiments, the present disclosure provides a pharmaceutical composition comprising a compound of formula I or I′, or a pharmaceutically acceptable salt thereof, and a pharmaceutically acceptable carrier, adjuvant, or diluent.

In some embodiments, the present disclosure provides a method of treating a JAK2-mediated disorder comprising administering to a patient in need thereof a compound of formula I or I′, or composition comprising said compound.

In some embodiments, the present disclosure provides a process for providing a compound of formula I or I′, or synthetic intermediates thereof.

In some embodiments, the present disclosure provides a process for providing pharmaceutical compositions comprising compounds of formula I or I′.

### DETAILED DESCRIPTION

### 1. General Description of Certain Embodiments of the Disclosure

Compounds of the present disclosure, and pharmaceutical compositions thereof, are useful as inhibitors of JAK2. In some embodiments, the present disclosure provides a compound of formula I:

Cy^{A}-L¹-Cy^{B} I

or a pharmaceutically acceptable salt thereof, wherein:

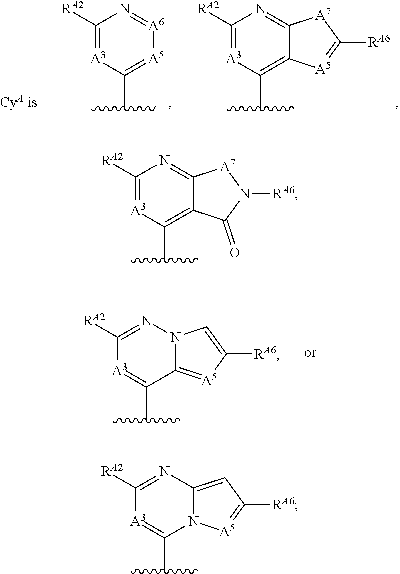

L¹ is —NH—; or

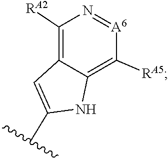

Cy^{A}-L¹- taken together are
Cy^{B} is

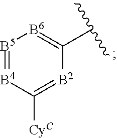

R^{A2} is hydrogen, or —NHR^{A2A}
A³ is N, CH, or C(R^{A3});
A⁵ is N, CH, or C(R^{A5});
A⁶ is N or CH;
A⁷ is NH, S, or CH₂;
B² is N, CH or C(R^{B2});
B⁴ is N, CH or C(R^{B4});
B⁵ is N, CH or C(R^{B5});
B⁶ is N or CH;
Cy^{C} is phenyl, or a 5-6 membered monocyclic heteroaryl ring having 1-4 heteroatoms independently selected from nitrogen, oxygen, and sulfur; wherein Cy^{C} is substituted by R^{C1} and 0-4 instances of R^{C2};
R^{C1} is

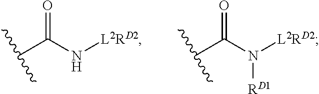

a 3-7 membered saturated or partially unsaturated heterocyclic ring having 1-4 heteroatoms independently selected from nitrogen, oxygen, or sulfur; or a 3-7 membered saturated or partially unsaturated carbocyclic ring; wherein said ring is substituted by -L²R^{D2} and 0-4 instances of R^{CA},
L² is —CH₂—, —CH(R^{L2})—, —C(R^{L2})₂—, —C(O)—, —O—, or —N(R^{L2})—;
each instance of R^{A2A}, R^{A3}, R^{A5}, R^{A6}, R^{B2}, R^{B4}, R^{B5}, R^{C2}, R^{CA}, R^{D1}, and R^{L2} is independently R^{A} or R^{B}, and is substituted by 0-4 instances of R^{C};
R^{D2} is R^{B}, substituted by 0-4 instances of R^{D2A}.
each instance of R^{D2A} is independently R^{C}; or an instance of R^{D2A} and R^{D1} are taken together with their intervening atoms to form a saturated or partially unsaturated 3-7 membered ring fused to R^{D2};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, the present disclosure provides a compound of formula I′:

Cy^{A}-L¹-Cy^{B} I′

or a pharmaceutically acceptable salt thereof, wherein:
Cy^{A} is

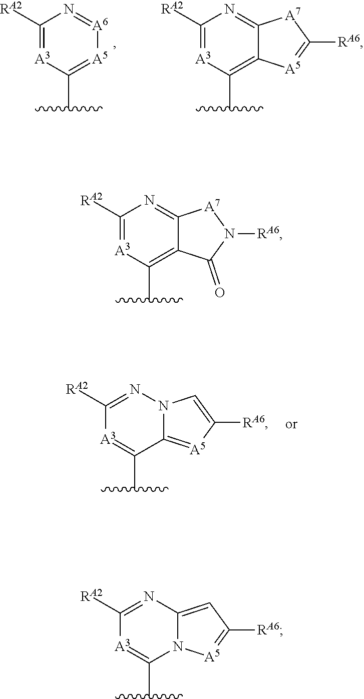

L¹ is —NH—; or
   Cy^{A}-L¹- taken together are

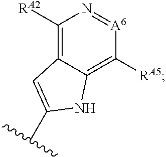

Cy^{B} is

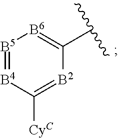

R^{A2} is hydrogen, or —NHR^{A2A}
A³ is N, CH, or C(R^{A3});
A⁵ is N, CH, or C(R^{A5});
A⁶ is N or CH;
A⁷ is NH, S, or CH₂;
B² is N, CH or C(R^{B2});
B⁴ is N, CH or C(R^{B4});
B⁵ is N, CH or C(R^{B5});
B⁶ is N or CH;
Cy^{C} is phenyl, or a 5-6 membered monocyclic heteroaryl ring having 1-4 heteroatoms independently selected from nitrogen, oxygen, and sulfur; wherein Cy^{C} is substituted by R^{C1} and 0-4 instances of R^{C2};
R^{C1} is

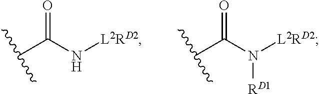

a 3-7 membered saturated or partially unsaturated heterocyclic ring having 1-4 heteroatoms independently selected from nitrogen, oxygen, or sulfur; or a 3-7 membered saturated or partially unsaturated carbocyclic ring; wherein said ring is substituted by -L²R^{D2} and 0-4 instances of R^{CA},
L² is —CH₂—, —CH(R^{L2})—, —C(R^{L2})₂—, —C(O)—, —O—, or —N(R^{L2})—;
each instance of R^{A2A}, R^{A3}, R^{A5}, R^{A6}, R^{B2}, R^{B4}, R^{B5}, R^{C2}, R^{CA}, R^{D1}, and R^{L2} is independently R^{A} or R^{B}, and is substituted by 0-4 instances of R^{C};
R^{D2} is R^{B}, substituted by 0-4 instances of R^{D2A},
each instance of R^{D2A} is independently R^{C}; or an instance of R^{D2A} and R^{D1} are taken together with their intervening atoms to form a saturated or partially unsaturated 3-7 membered ring fused to R^{D2};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “C₁₋₈ (or C₁₋₆, or C₁₋₄) bivalent saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but is not limited to, phenyl, biphenyl, naphthyl, anthracyl and the like, which may bear one or more substituents.

The terms “heteroaryl” or “heteroaromatic”, unless otherwise defined, as used herein refers to a monocyclic aromatic 5-6 membered ring containing one or more heteroatoms, for example one to three heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

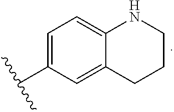 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings, and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

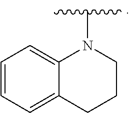 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

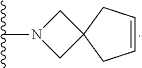 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

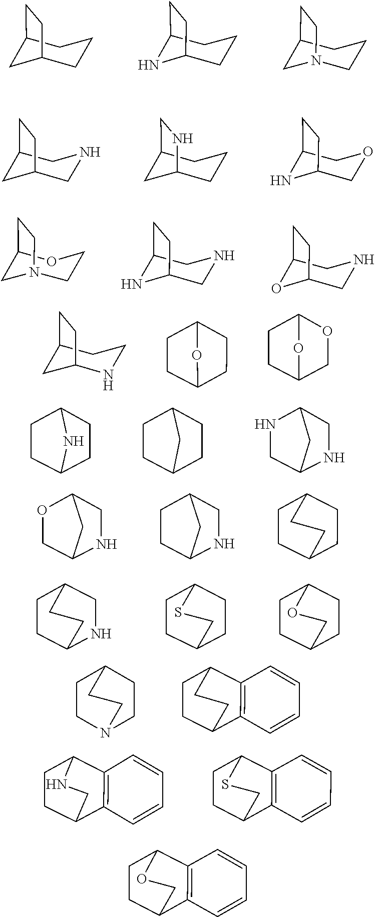

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘}S(O)₂NR^{∘}₂; —N(R^{∘}S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}. —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O (haloR^{•}), —CN, —C(O) OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O) OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds of the disclosure are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds of the disclosure are within the scope of the disclosure.

Such compounds are useful, for example, as analytical tools, as probes in biological assays, or as therapeutic agents in accordance with the present disclosure. In certain embodiments, for example, a warhead moiety, R^{W}, of a provided compound comprises one or more deuterium atoms.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66 (1), 1; and Gould, P. L., Int. J. Pharmaceutics 1986, 33, 201-217; (each hereby incorporated by reference in its entirety).

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to JAK2 signaling, or one or more symptoms thereof, prevent the advancement of conditions or symptoms related to afflictions related to JAK2 signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to JAK2 signaling activity or that may otherwise be relieved by the compounds and/or compositions of the disclosure.

### 3. Description of Exemplary Embodiments

As described above, in some embodiments, the present disclosure provides a compound of formula I:

Cy^{A}-L¹-Cy^{B} I

or a pharmaceutically acceptable salt thereof, wherein:
Cy^{A} is

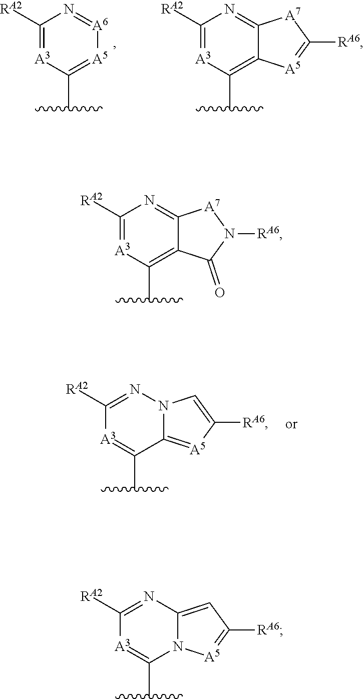

L¹ is —NH—; or

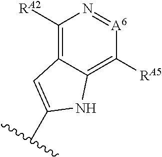

Cy^{A}-L¹- taken together are
Cy^{B} is

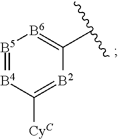

R^{A2} is hydrogen, or —NHR^{A2A};
A³ is N, CH, or C(R^{A3});
A⁵ is N, CH, or C(R^{A5});
A⁶ is N or CH;
A⁷ is NH, S, or CH₂;
B² is N, CH or C(R^{B2});
B⁴ is N, CH or C(R^{B4});
B⁵ is N, CH or C(R^{B5});
B⁶ is N or CH;
Cy^{C} is phenyl, or a 5-6 membered monocyclic heteroaryl ring having 1-4 heteroatoms independently selected from nitrogen, oxygen, and sulfur; wherein Cy^{C} is substituted by R^{C1} and 0-4 instances of R^{C2};
R^{C1} is

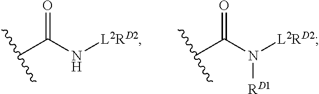

a 3-7 membered saturated or partially unsaturated heterocyclic ring having 1-4 heteroatoms independently selected from nitrogen, oxygen, or sulfur; or a 3-7 membered saturated or partially unsaturated carbocyclic ring; wherein said ring is substituted by -L²R^{D2} and 0-4 instances of R^{CA},
L² is —CH₂—, —CH(R^{L2})—, —C(R^{L2})₂—, —C(O)—, —O—, or —N(R^{L2})—;
each instance of R^{A2A}, R^{A3}, R^{A5}, R^{A6}, R^{B2}, R^{B4}, R^{B5}, R^{C2}, R^{CA}, R^{D1}, and R^{L2} is independently R^{A} or R^{B}, and is substituted by 0-4 instances of R^{C};
R^{D2} is R^{B}, substituted by 0-4 instances of R^{D2A}.
each instance of R^{D2A} is independently R^{C}; or an instance of R^{D2A} and R^{D1} are taken together with their intervening atoms to form a saturated or partially unsaturated 3-7 membered ring fused to R^{D2};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, the present disclosure provides a compound of formula I′:

Cy^{A}-L¹-Cy^{B} I′
or a pharmaceutically acceptable salt thereof, wherein:
Cy^{A} is

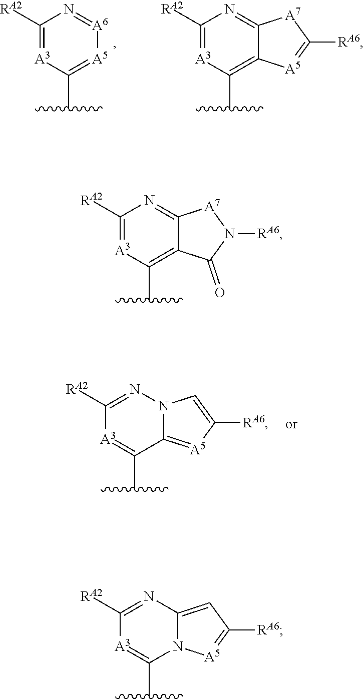

L¹ is —NH—; or
   Cy^{A}-L¹- taken together are

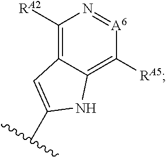

Cy^{B} is

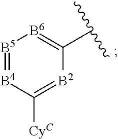

R^{A2} is hydrogen, or —NHR^{A2A}
A³ is N, CH, or C(R^{A3});
A⁵ is N, CH, or C(R^{A5});
A⁶ is N or CH;
A⁷ is NH, S, or CH₂;
B² is N, CH or C(R^{B2});
B⁴ is N, CH or C(R^{B4});
B⁵ is N, CH or C(R^{B5});
B⁶ is N or CH;
Cy^{C} is phenyl, or a 5-6 membered monocyclic heteroaryl ring having 1-4 heteroatoms independently selected from nitrogen, oxygen, and sulfur; wherein Cy^{C} is substituted by R^{C1} and 0-4 instances of R^{C2};
R^{C1} is

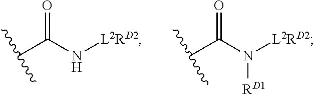

a 3-7 membered saturated or partially unsaturated heterocyclic ring having 1-4 heteroatoms independently selected from nitrogen, oxygen, or sulfur; or a 3-7 membered saturated or partially unsaturated carbocyclic ring; wherein said ring is substituted by -L²R^{D2} and 0-4 instances of R^{CA},
L² is —CH₂—, —CH(R^{L2})—, —C(R^{L2})₂—, —C(O)—, —O—, or —N(R^{L2})—;
each instance of R^{A2A}, R^{A3}, R^{A5}, R^{A6}, R^{B2}, R^{B4}, R^{B5}, R^{C2}, R^{CA}, R^{D1}, and R^{L2} is independently R^{A} or R^{B}, and is substituted by 0-4 instances of R^{C};
R^{D2} is R^{B}, substituted by 0-4 instances of R^{D2A},
each instance of R^{D2A} is independently R^{C}; or an instance of R^{D2A} and R^{D1} are taken together with their intervening atoms to form a saturated or partially unsaturated 3-7 membered ring fused to R^{D2};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
   two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur.

As defined generally above, Cy^{A} is

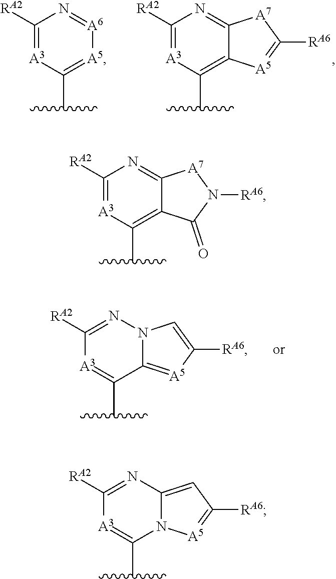

or Cy^{A}-L¹-taken together are

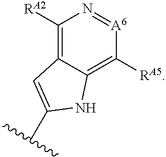

In some embodiments, Cy^{A} is

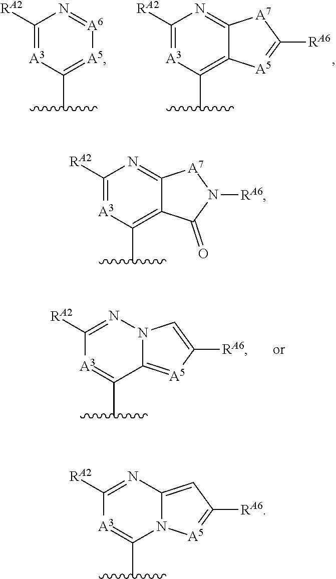

In some embodiments, Cy^{A} is

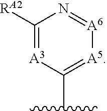

In some embodiments, Cy^{A} is,

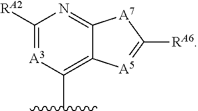

In some embodiments, Cy^{A} is,

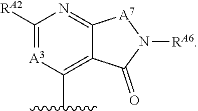

In some embodiments, Cy^{A} is

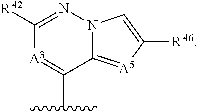

In some embodiments, Cy^{A} is

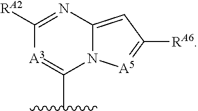

In some embodiments, Cy^{A}-L¹- taken together are

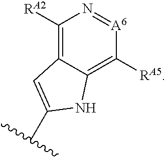

In some embodiments, Cy^{A} taken together with its A³, A⁵, A⁶, and R^{A2} substituents is

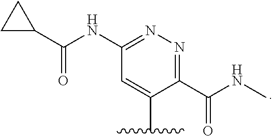 [SMILES: *c1cc(NC(=O)C2CC2)nnc1C(=O)NC]

In some embodiments, Cy^{A} taken together with its A³, A⁵, A⁶, and R^{A2} substituents is

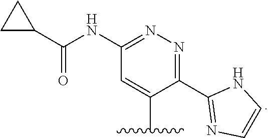 [SMILES: *c1cc(NC(=O)C2CC2)nnc1-c1ncc[nH]1]

In some embodiments, Cy^{A} taken together with its A³, A⁵, A⁶, and R^{A2} substituents is

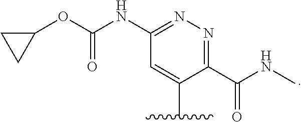 [SMILES: *c1cc(NC(=O)OC2CC2)nnc1C(=O)NC]

In some embodiments, Cy^{A} taken together with its A³, A⁵, A⁶, and R^{A2} substituents is

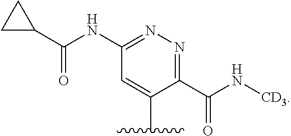 [SMILES: *c1cc(NC(=O)C2CC2)nnc1C(=O)NC([2H])([2H])[2H]]

In some embodiments, Cy^{A} taken together with its A³, A⁵, R^{A2}, and R^{A6} substituents is

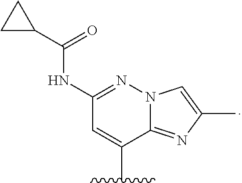 [SMILES: *c1cc(NC(=O)C2CC2)nn2cc(C)nc12]

In some embodiments, Cy^{A} taken together with its A³, A⁵, R^{A2}, and R^{A6} substituents is

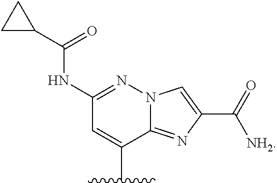 [SMILES: *c1cc(NC(=O)C2CC2)nn2cc(C(N)=O)nc12]

In some embodiments, Cy^{A} taken together with its A³, A⁵, R^{A2}, and R^{A6} substituents is

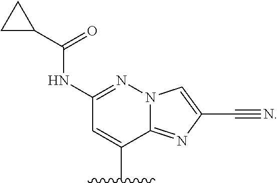 [SMILES: *c1cc(NC(=O)C2CC2)nn2cc(C#N)nc12]

As defined generally above, L¹ is —NH—. In some embodiments, L¹ is —NH—. In some embodiments, L¹ is selected from the groups depicted in the compounds in Table 1.

As defined generally above, Cy^{B} is

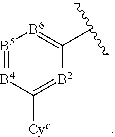

In some embodiments, Cy^{B} taken together with its B², B⁴, B⁵, and B⁶ substituents is

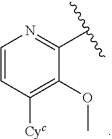

In some embodiments, Cy^{B} taken together with its B², B⁴, B⁵, and B⁶ substituents is

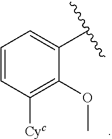

In some embodiments, Cy^{B} taken together with its B², B⁴, B⁵, and B⁶ substituents is

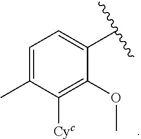

In some embodiments, Cy^{B} taken together with its B², B⁴, B⁵, and B⁶ substituents is

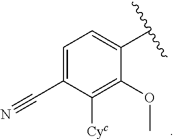

In some embodiments, Cy^{B} is selected from the groups depicted in the compounds in Table 1.

As defined generally above, R^{A2} is hydrogen, or —NHR^{A2A}. In some embodiments, R^{A2} is hydrogen. In some embodiments, R^{A2} is —NHR^{A2A}. In some embodiments, R^{A2} is selected from the groups depicted in the compounds in Table 1.

As defined generally above, R^{A2A} is R^{A} or R^{B}, and is substituted by 0-4 instances of R^{C}. In some embodiments, R^{A2A} is —C(O)R, —C(O)OR, —C(O)NR₂, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur. In some embodiments, R^{A2A} is —C(O)R. In some embodiments, R^{A2A} is

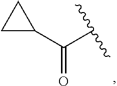 [SMILES: *C(=O)C1CC1]

substituted by 0-4 instances of R^{C}. In some embodiments, R^{A2A} is —C(O)OR. In some embodiments, R^{A2A} is

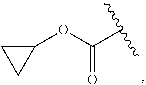 [SMILES: *C(=O)OC1CC1]

substituted by 0-4 instances of R^{C}. In some embodiments, R^{A2A} is —C(O)NR₂. In some embodiments, R^{A2A} is a 5-6 membered monocyclic heteroaryl ring having 1-4 heteroatoms independently selected from nitrogen, oxygen, and sulfur. In some embodiments, R^{A2A} is an 8-10 membered bicyclic heteroaryl ring having 1-4 heteroatoms independently selected from nitrogen, oxygen, and sulfur. In some embodiments, R^{A2A} is selected from the groups depicted in the compounds in Table 1.

As defined generally above, A³ is N, CH, or C(R^{A3}). In some embodiments, A³ is N. In some embodiments, A³ is CH. In some embodiments, A³ is C(R^{A3}). In some embodiments, A³ is selected from the groups depicted in the compounds in Table 1.

As defined generally above, each instance of R^{A3} is independently R^{A} or R^{B}, and is substituted by 0-4 instances of R^{C}. In some embodiments, R^{A3} is R^{A} substituted by 0-4 instances of R^{C}. In some embodiments, R^{A3} is R^{B} substituted by 0-4 instances of R^{C}. In some embodiments, R^{A3} is selected from the groups depicted in the compounds in Table 1.

As defined generally above, A⁵ is N, CH, or C(R^{A5}). In some embodiments, A⁵ is N. In some embodiments, A⁵ is CH. In some embodiments, A⁵ is C(R^{A5}). In some embodiments, A⁵ is selected from the groups depicted in the compounds in Table 1.

As defined generally above, R^{A5} is R^{A} or R^{B}, and is substituted by 0-4 instances of R^{C}. In some embodiments, R^{A5} is R^{A}, and is substituted by 0-4 instances of R^{C}. In some embodiments, R^{A5} is R^{B}, and is substituted by 0-4 instances of R^{C}. In some embodiments, R^{A5} is —C(O)NH₂, —C(O)NHMe, or —C(O)NHCD₃. In some embodiments, R^{A5} is —C(O)NH₂. In some embodiments, R^{A5} is —C(O)NHMe, or —C(O)NHCD₃. In some embodiments, R^{A5} is —C(O)NHMe. In some embodiments, R^{A5} is-CN. In some embodiments, R^{A5} is a 5-6 membered monocyclic heteroaryl ring having 1-4 heteroatoms independently selected from nitrogen, oxygen, and sulfur, substituted with 0-4 instances of R^{C}. In some embodiments, R^{A5} is selected from the groups depicted in the compounds in Table 1.

As defined generally above, A⁶ is N or CH. In some embodiments, A⁶ is N. In some embodiments, A⁶ is CH. In some embodiments, A⁶ is selected from the groups depicted in the compounds in Table 1.

As defined generally above, R^{A6} is R^{A} or R^{B}, and is substituted by 0-4 instances of R^{C}. In some embodiments, R^{A6} is R^{A} substituted by 0-4 instances of R^{C}. In some embodiments, R^{A6} is R^{B}, substituted by 0-4 instances of R^{C}. In some embodiments, R^{A6} is halogen, CH₃, CD₃, CHF₂, or CF₃. In some embodiments, R^{A6} is CH₃. In some embodiments, R^{A6} is CD₃. In some embodiments, R^{A6} is CHF₂. In some embodiments, R^{A6} is selected from the groups depicted in the compounds in Table 1.

As defined generally above, A⁷ is NH, S, or CH₂. In some embodiments, A⁷ is NH. In some embodiments, A⁷ is S. In some embodiments, A⁷ is CH₂. In some embodiments, A⁷ is selected from the groups depicted in the compounds in Table 1.

As defined generally above, B² is N, CH or C(R^{B2}). In some embodiments, B² is N. In some embodiments, B² is CH. In some embodiments, B² is C(R^{B2}). In some embodiments, B² is selected from the groups depicted in the compounds in Table 1.

As defined generally above, each instance of R^{B2} is independently R^{A} or R^{B}, and is substituted by 0-4 instances of R^{C}. In some embodiments, R^{B2} is R^{A} substituted by 0-4 instances of R^{C}. In some embodiments, R^{B2} is R^{B} substituted by 0-4 instances of R^{C}. In some embodiments, R^{B2} is —OCH₃. In some embodiments, R^{B2} is selected from the groups depicted in the compounds in Table 1.

As defined generally above, B⁴ is N, CH or C(R^{B4}). In some embodiments, B⁴ is N. In some embodiments, B⁴ is CH. In some embodiments, B⁴ is C(R^{B4}). In some embodiments, B⁴ is selected from the groups depicted in the compounds in Table 1.

As defined generally above, each instance of R^{B4} is independently R^{A} or R^{B}, and is substituted by 0-4 instances of R^{C}. In some embodiments, R^{B4} is R^{A} substituted by 0-4 instances of R^{C}. In some embodiments, R^{B4} is R^{B} substituted by 0-4 instances of R^{C}. In some embodiments, R^{B4} is CH₃. In some embodiments, R^{B4} is CN. In some embodiments, R^{B4} is halogen. In some embodiments, R^{B4} is chloro. In some embodiments, R^{B4} is selected from the groups depicted in the compounds in Table 1.

As defined generally above, B⁵ is N, CH or C(R^{B5}). In some embodiments, B⁵ is N. In some embodiments, B⁵ is CH. In some embodiments, B⁵ is C(R^{B5}). In some embodiments, B⁵ is selected from the groups depicted in the compounds in Table 1.

As defined generally above, each instance of R^{B5} is independently R^{A} or R^{B}, and is substituted by 0-4 instances of R^{C}. In some embodiments, R^{B5} is R^{A} substituted by 0-4 instances of R^{C}. In some embodiments, R^{B5} is R^{B} substituted by 0-4 instances of R^{C}. In some embodiments, R^{B5} is halogen. In some embodiments, R^{B5} is fluoro. In some embodiments, R^{B5} is selected from the groups depicted in the compounds in Table 1.

As defined generally above, B⁶ is N or CH. In some embodiments, B⁶ is N. In some embodiments, B⁶ is CH. In some embodiments, B⁶ is selected from the groups depicted in the compounds in Table 1.

As defined generally above, Cy^{C} is phenyl, or a 5-6 membered monocyclic heteroaryl ring having 1-4 heteroatoms independently selected from nitrogen, oxygen, and sulfur; wherein Cy^{C} is substituted by R^{C1} and 0-4 instances of R^{C2}. In some embodiments, Cy^{C} is phenyl substituted by R^{C1} and 0-4 instances of R^{C2}. In some embodiments, Cy^{C} is a 5-6 membered monocyclic heteroaryl ring having 1-4 heteroatoms independently selected from nitrogen, oxygen, and sulfur; substituted by R^{C1} and 0-4 instances of R^{C2}. In some embodiments, Cy^{C} is a 5 membered monocyclic heteroaryl ring having 1-4 heteroatoms independently selected from nitrogen, oxygen, and sulfur; substituted by R^{C1} and 0-4 instances of R^{C2}. In some embodiments, Cy^{C} is a 6 membered monocyclic heteroaryl ring having 1-4 heteroatoms independently selected from nitrogen, oxygen, and sulfur; substituted by R^{C1} and 0-4 instances of R^{C2}.

In some embodiments, Cy^{C} is

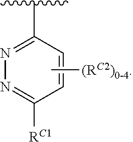

In some embodiments, Cy^{C} is

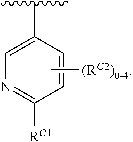

In some embodiments, Cy^{C} is

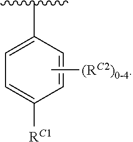

In some embodiments, Cy^{C} is

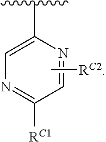

In some embodiments, Cy^{C} is

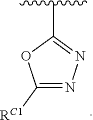

In some embodiments, Cy^{C} is

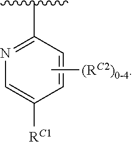

In some embodiments, Cy^{C} is

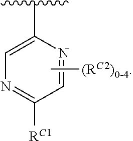

In some embodiments, Cy^{C} is

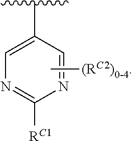

In some embodiments, Cy^{C} is

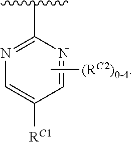

In some embodiments, Cy^{C} is

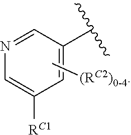

In some embodiments, Cy^{C} is

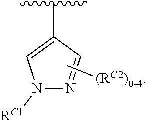

In some embodiments, Cy^{C} is

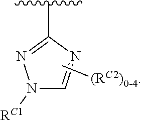

In some embodiments, Cy^{C} is

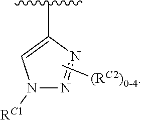

In some embodiments, Cy^{C} is

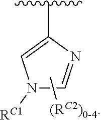

In some embodiments, Cy^{C} is

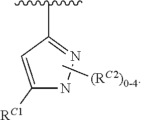

In some embodiments, Cy^{C} is selected from the groups depicted in the compounds in Table 1.

As defined generally above, R^{C1} is

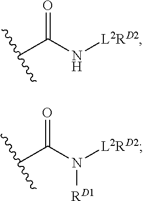

a 3-7 membered saturated or partially unsaturated heterocyclic ring having 1-4 heteroatoms independently selected from nitrogen, oxygen, or sulfur; or a 3-7 membered saturated or partially unsaturated carbocyclic ring; wherein said ring is substituted by -L²R^{D2} and 0-4 instances of R^{CA}. In some embodiments, R^{C1} is

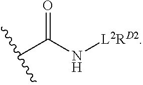

In some embodiments, R^{C1} is

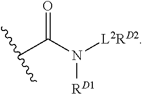

In some embodiments, R^{C1} is a 3-7 membered saturated or partially unsaturated heterocyclic ring having 1-4 heteroatoms independently selected from nitrogen, oxygen, or sulfur; or a 3-7 membered saturated or partially unsaturated carbocyclic ring; wherein said ring is substituted by -L²R^{D2} and 0-4 instances of R^{CA}. In some embodiments, R^{C1} is a 3-4 membered saturated or partially unsaturated heterocyclic ring having 1-4 heteroatoms independently selected from nitrogen, oxygen, or sulfur; or a 3-4 membered saturated or partially unsaturated carbocyclic ring; wherein said ring is substituted by -L²R^{D2} and 0-4 instances of R^{CA}. In some embodiments, R^{C1} is a 3-4 membered saturated or partially unsaturated heterocyclic ring having 1-4 heteroatoms independently selected from nitrogen, oxygen, or sulfur, wherein said ring is substituted by -L²R^{D2} and 0-4 instances of R^{CA}. In some embodiments, R^{C1} is a 3-4 membered saturated or partially unsaturated carbocyclic ring; wherein said ring is substituted by -L²R^{D2} and 0-4 instances of R^{CA}. In some embodiments, R^{C1} is a 4 membered saturated heterocyclic ring having 1-4 heteroatoms independently selected from nitrogen, oxygen, or sulfur, wherein said ring is substituted by -L²R^{D2} and 0-4 instances of R^{CA}. In some embodiments, R^{C1} is a 4 membered saturated carbocyclic ring having 1-4 heteroatoms independently selected from nitrogen, oxygen, or sulfur, wherein said ring is substituted by -L²R^{D2} and 0-4 instances of R^{CA}. In some embodiments, R^{C1} is azetidine, substituted by -L²R^{D2} and 0-4 instances of R^{CA}.

In some embodiments, R^{C1} is

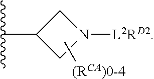

In some embodiments, R^{C1} is

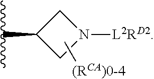

In some embodiments, R^{C1} is

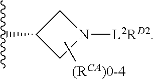

In some embodiments, R^{C1} is

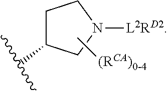

In some embodiments, R^{C1} is

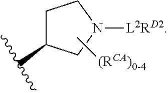

In some embodiments, R^{C1} is

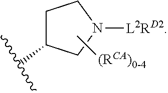

In some embodiments, R^{C1} is

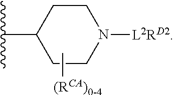

In some embodiments, R^{C1} is

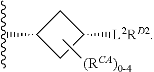

In some embodiments, R^{C1} is

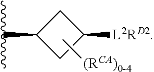

In some embodiments, R^{C1} is

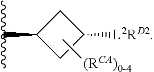

In some embodiments, R^{C1} is

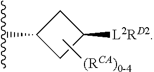

In some embodiments, R^{C1} is

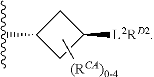

In some embodiments, R^{C1} is selected from the groups depicted in the compounds in Table 1.

As defined generally above, each instance of R^{CA} is independently R^{A} or R^{B}, and is substituted by 0-4 instances of R^{C}.

In some embodiments, R^{CA} is R^{A} substituted by 0-4 instances of R^{C}. In some embodiments, R^{CA} is R^{B} substituted by 0-4 instances of R^{C}.

In some embodiments, R^{CA} is

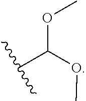 [SMILES: *C(OC)OC]

In some embodiments, R^{CA} is

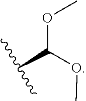

In some embodiments, R^{CA} is

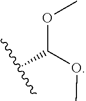

In some embodiments, R^{CA} is

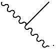

In some embodiments, R^{CA} is

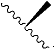

In some embodiments, R^{CA} is

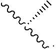

In some embodiments, R^{CA} is

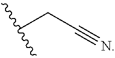 [SMILES: *CC#N]

In some embodiments, R^{CA} is

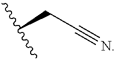

In some embodiments, R^{CA} is

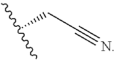

In some embodiments, R^{CA} is selected from the groups depicted in the compounds in Table 1.

As defined generally above, L² is —CH₂—, —CH(R^{L2})—, —C(R^{L2})₂—, —C(O)—, —O—, or —N(R^{L2})—. In some embodiments, L² is —CH₂—, —CH(R^{L2})—, or —C(R^{L2})₂—. In some embodiments, L² is —CH₂—, or —CH(R^{L2})—. In some embodiments, L² is —CH₂—. In some embodiments, L² is —CH(R^{L2})—. In some embodiments, L² is —C(R^{L2})₂—. In some embodiments, L² is —C(O)—. In some embodiments, L² is —O—. In some embodiments, L² is —N(R^{L2})—.

In some embodiments, L² is

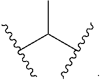 [SMILES: *C(*)C]

In some embodiments, L² is

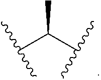

In some embodiments, L² is

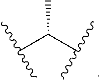

In some embodiments, L² is

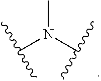 [SMILES: *N(*)C]

In some embodiments, L² is

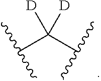 [SMILES: *C(*)([2H])[2H]]

In some embodiments, L² is

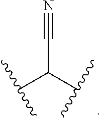 [SMILES: *C(*)C#N]

In some embodiments, L² is

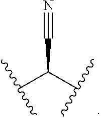

In some embodiments, L² is

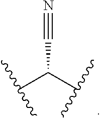

In some embodiments, L² is

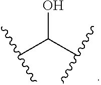 [SMILES: *C(*)O]

In some embodiments, L² is

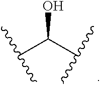

In some embodiments, L² is

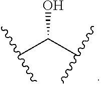

In some embodiments, L² is

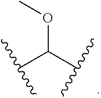 [SMILES: *C(*)OC]

In some embodiments, L² is

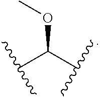

In some embodiments, L² is

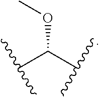

In some embodiments, L² is selected from the groups depicted in the compounds in Table 1.

As defined generally above, each instance of R^{C2} is independently R^{A} or R^{B}, and is substituted by 0-4 instances of R^{C}.

In some embodiments, R^{C2} is R^{A} substituted by 0-4 instances of R^{C}. In some embodiments, R^{C2} is R^{B} substituted by 0-4 instances of R^{C}.

In some embodiments, R^{C2} is CH₃. In some embodiments, R^{C2} is CN. In some embodiments, R^{C2} is halogen. In some embodiments, R^{C2} is fluoro. In some embodiments, R^{C2} is CF₃. In some embodiments, R^{C2} is CHF₂. In some embodiments, R^{C2} is OCH₃. In some embodiments, R^{C2} is

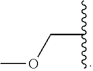 [SMILES: *COC]

In some embodiments, R^{C2} is

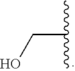 [SMILES: *CO]

In some embodiments, R^{C2} is

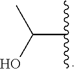 [SMILES: *C(C)O]

In some embodiments, R^{C2} is

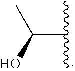 [SMILES: *[C@H](C)O]

In some embodiments, R^{C2} is

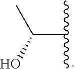 [SMILES: *[C@@H](C)O]

In some embodiments, R^{C2} is

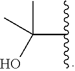 [SMILES: *C(C)(C)O]

In some embodiments, R^{C2} is

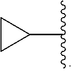 [SMILES: *C1CC1]

In some embodiments, R^{C2} is selected from the groups depicted in the compounds in Table 1.

As defined generally above, each instance of R^{CA} is independently R^{A} or R^{B}, and is substituted by 0-4 instances of R^{C}.

In some embodiments, R^{CA} is R^{A} substituted by 0-4 instances of R^{C}. In some embodiments, R^{CA} is R^{B} substituted by 0-4 instances of R^{C}.

In some embodiments, R^{CA} is selected from the groups depicted in the compounds in Table 1.

As defined generally above, each instance of R^{D1} is independently R^{A} or R^{B}, and is substituted by 0-4 instances of R^{C}.

In some embodiments, R^{D1} is R^{A} substituted by 0-4 instances of R^{C}. In some embodiments, R^{D1} is R^{B} substituted by 0-4 instances of R^{C}.

In some embodiments, R^{D1} is CH₃. In some embodiments, R^{D1} is CD₃. In some embodiments, R^{D1} is OCH₃. In some embodiments, R^{D1} is

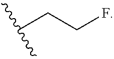 [SMILES: *CCF]

In some embodiments, R^{D1} is

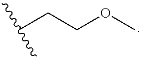 [SMILES: *CCOC]

In some embodiments, R^{D1} is selected from the groups depicted in the compounds in Table 1.

As defined generally above, each instance of R^{L2} is independently R^{A} or R^{B}, and is substituted by 0-4 instances of R^{C}.

In some embodiments, R^{L2} is R^{A} substituted by 0-4 instances of R^{C}. In some embodiments, R^{L2} is R^{B} substituted by 0-4 instances of R^{C}.

In some embodiments, R^{L2} is selected from the groups depicted in the compounds in Table 1.

As defined generally above, R^{D2} is R^{B}, substituted by 0-4 instances of R^{D2A}. In some embodiments, R^{D2} is phenyl, or a 5-6 membered monocyclic heteroaryl ring having 1-4 heteroatoms independently selected from nitrogen, oxygen, and sulfur, and is substituted by 0-4 instances of R^{D2A}. In some embodiments, R^{D2} is phenyl, substituted by 0-4 instances of R^{D2A}. In some embodiments, R^{D2} is a 5-6 membered monocyclic heteroaryl ring having 1-4 heteroatoms independently selected from nitrogen, oxygen, and sulfur, substituted by 0-4 instances of R^{D2A}. In some embodiments, R^{D2} is a 5 membered monocyclic heteroaryl ring having 1-4 heteroatoms independently selected from nitrogen, oxygen, and sulfur, and is substituted by 0-4 instances of R^{D2A}. In some embodiments, R^{D2} is a 6 membered monocyclic heteroaryl ring having 1-4 heteroatoms independently selected from nitrogen, oxygen, and sulfur, and is substituted by 0-4 instances of R^{D2A}. In some embodiments, R^{D2} is pyridyl, substituted by 0-4 instances of R^{D2A}. In some embodiments, R^{D2} is thiazolyl, substituted by 0-4 instances of R^{D2A}. In some embodiments, R^{D2} is pyrazolyl, substituted by 0-4 instances of R^{D2A}.

In some embodiments, R^{D2} is

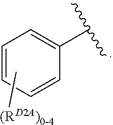

In some embodiments, R^{D2} is

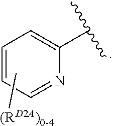

In some embodiments, R^{D2} is

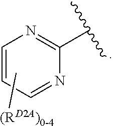

In some embodiments, R^{D2} is

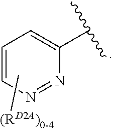

In some embodiments, R^{D2} is

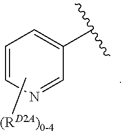

In some embodiments, R^{D2} is

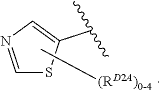

In some embodiments, R^{D2} is

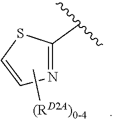

In some embodiments, R^{D2} is

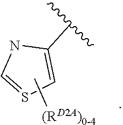

In some embodiments, R^{D2} is

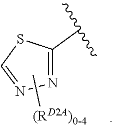

In some embodiments, R^{D2} is

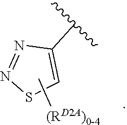

In some embodiments, R^{D2} is

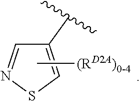

In some embodiments, R^{D2} is

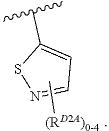

In some embodiments, R^{D2} is

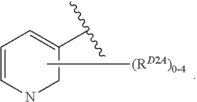

In some embodiments, R^{D2} is

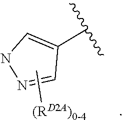

In some embodiments, R^{D2} is

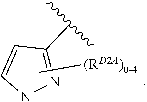

In some embodiments, R^{D2} is

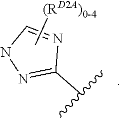

In some embodiments, R^{D2} is

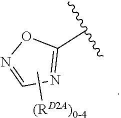

In some embodiments, R^{D2} is

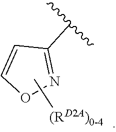

In some embodiments, R^{D2} is

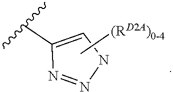

In some embodiments, R^{D2} is

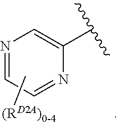

In some embodiments, R^{D2} is

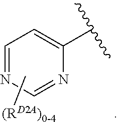

In some embodiments, R^{D2} is

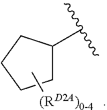

In some embodiments, R^{D2} is

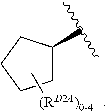

In some embodiments, R^{D2} is

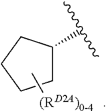

In some embodiments, R^{D2} is

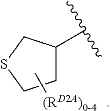

In some embodiments, R^{D2} is

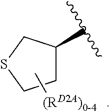

In some embodiments, R^{D2} is

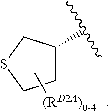

In some embodiments, R^{D2} is

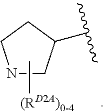

In some embodiments, R^{D2} is

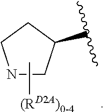

In some embodiments, R^{D2} is

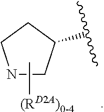

In some embodiments, R^{D2} is

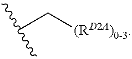

In some embodiments, R^{D2} is

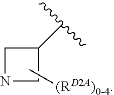

In some embodiments, R^{D2} is

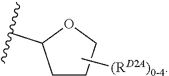

In some embodiments, R^{D2} is

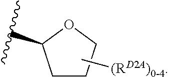

In some embodiments, R^{D2} is

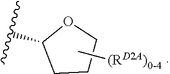

In some embodiments, R^{D2} is

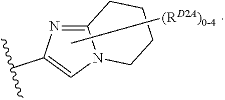

In some embodiments, R^{D2} is

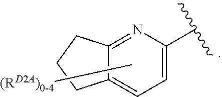

In some embodiments, R^{D2} is

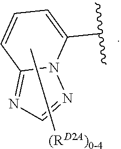

In some embodiments, R^{D2} is

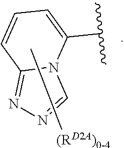

In some embodiments, R^{D2} is

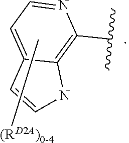

In some embodiments, R^{D2} is

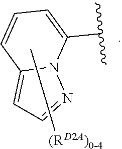

In some embodiments, R^{D2} is

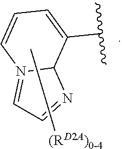

In some embodiments, R^{D2} is

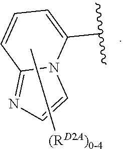

In some embodiments, R^{D2} is

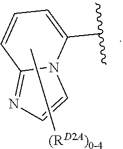

In some embodiments, R^{D2} is

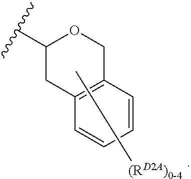

In some embodiments, R^{D2} is

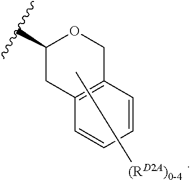

In some embodiments, R^{D2} is

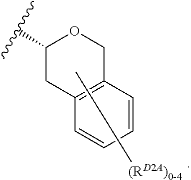

In some embodiments, R^{D2} is

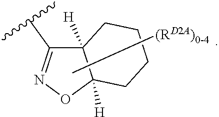

In some embodiments, R^{D2} is

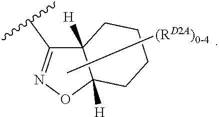

In some embodiments, R^{D2} is

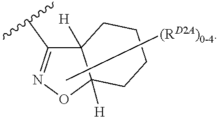

In some embodiments, R^{D2} is selected from the groups depicted in the compounds in Table 1.

As defined generally above, each instance of R^{D2A} is independently R^{C}; or an instance of R^{D2A} and R^{D1} are taken together with their intervening atoms to form a saturated or partially unsaturated 3-7 membered ring fused to R^{D2}.

In some embodiments, R^{D2A} is R^{C}. In some embodiments, R_{D2A} and R^{D1} are taken together with their intervening atoms to form a saturated or partially unsaturated 3-7 membered ring fused to R_{D2}.

In some embodiments, R^{D2A} is halogen. In some embodiments, R^{D2A} is fluoro. In some embodiments, R_{D2A} is chloro. In some embodiments, R^{D2A} is bromo. In some embodiments, R^{D2A} is CN. In some embodiments, R^{D2A} is NH₂. In some embodiments, R^{D2A} is CH₃. In some embodiments, R^{D2A} is oxo. In some embodiments, R^{D2A} is OCHF₂.

In some embodiments, R^{D2A} is CF₃. In some embodiments, R^{D2A} is CHF₂. In some embodiments, R^{D2A} is OCH₃. In some embodiments, R^{D2A} is OCH₂CH₃. In some embodiments, R^{D2A} is CO₂H. In some embodiments, R^{D2A} is CHO. In some embodiments, R^{D2A} is OH. In some embodiments, R^{D2A} is

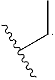 [SMILES: *CC]

In some embodiments, R^{D2A} is

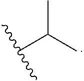 [SMILES: *C(C)C]

In some embodiments, R^{D2A} is

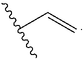 [SMILES: *C=C]

In some embodiments, R^{D2A} is

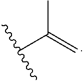 [SMILES: *C(=C)C]

In some embodiments, R^{D2A} is

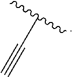 [SMILES: *C#C]

In some embodiments, R^{D2A} is

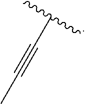 [SMILES: *C#CC]

In some embodiments, R^{D2A} is

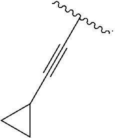 [SMILES: *C#CC1CC1]

In some embodiments, R^{D2A} is

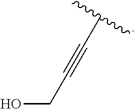 [SMILES: *C#CCO]

In some embodiments, R^{D2A} is

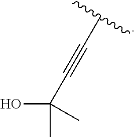 [SMILES: *C#CC(C)(C)O]

In some embodiments, R^{D2A} is

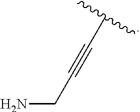 [SMILES: *C#CCN]

In some embodiments, R^{D2A} is

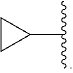 [SMILES: *C1CC1]

In some embodiments, R^{D2A} is

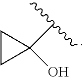 [SMILES: *C1(O)CC1]

In some embodiments, R^{D2A} is

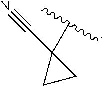 [SMILES: *C1(C#N)CC1]

In some embodiments, R^{D2A} is

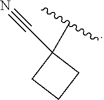 [SMILES: *C1(C#N)CCC1]

In some embodiments, R^{D2A} is

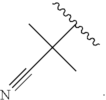 [SMILES: *C(C)(C)C#N]

In some embodiments, R^{D2A} is

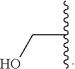 [SMILES: *CO]

In some embodiments, R^{D2A} is

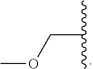 [SMILES: *COC]

In some embodiments, R^{D2A} is

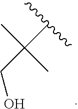 [SMILES: *C(C)(C)CO]

In some embodiments, R^{D2A} is

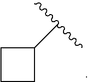 [SMILES: *C1CCC1]

In some embodiments, R^{D2A} is

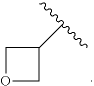 [SMILES: *C1COC1]

In some embodiments,R^{D2A} is

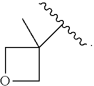 [SMILES: *C1(C)COC1]

In some embodiments, R^{D2A} is

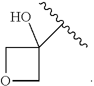 [SMILES: *C1(O)COC1]

In some embodiments, R^{D2A} is

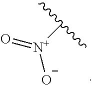 [SMILES: *[N+](=O)[O-]]

In some embodiments, R^{D2A} is

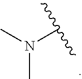 [SMILES: *N(C)C]

In some embodiments, R^{D2A} is

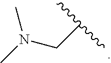 [SMILES: *CN(C)C]

In some embodiments, R^{D2A} is

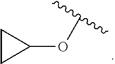 [SMILES: *OC1CC1]

In some embodiments, R^{D2A} is

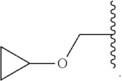 [SMILES: *COC1CC1]

In some embodiments, R^{D2A} is

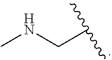 [SMILES: *CNC]

In some embodiments, R^{D2A} is

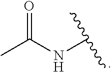 [SMILES: *NC(C)=O]

In some embodiments, R^{D2A} is

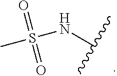 [SMILES: *NS(C)(=O)=O]

In some embodiments, R^{D2A} is

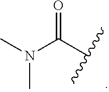 [SMILES: *C(=O)N(C)C]

In some embodiments, R^{D2A} is

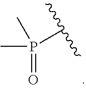 [SMILES: *P(C)(C)=O]

In some embodiments, R^{D2A} is

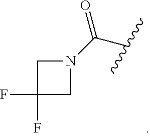 [SMILES: *C(=O)N1CC(F)(F)C1]

In some embodiments, R^{D2A} is

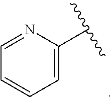 [SMILES: *c1ccccn1]

In some embodiments, R^{D2A} is

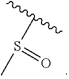 [SMILES: *S(C)=O]

In some embodiments, R^{D2A} is

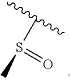 [SMILES: *[S@](C)=O]

In some embodiments, R^{D2A} is

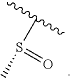 [SMILES: *[S@@](C)=O]

In some embodiments, R^{D2A} is

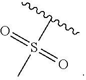 [SMILES: *S(C)(=O)=O]

In some embodiments, R^{D2A} is

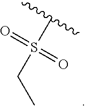 [SMILES: *S(=O)(=O)CC]

In some embodiments, R^{D2A} is

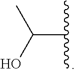 [SMILES: *C(C)O]

In some embodiments, R^{D2A} is

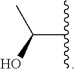 [SMILES: *[C@H](C)O]

In some embodiments, R^{D2A} is

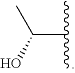 [SMILES: *[C@@H](C)O]

In some embodiments, R^{D2A} is

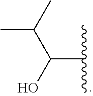 [SMILES: *C(O)C(C)C]

In some embodiments, R^{D2A} is

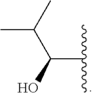 [SMILES: *[C@@H](O)C(C)C]

In some embodiments, R^{D2A} is

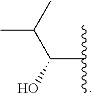 [SMILES: *[C@H](O)C(C)C]

In some embodiments, R^{D2A} is

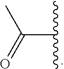 [SMILES: *C(C)=O]

In some embodiments, R^{D2A} is

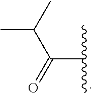 [SMILES: *C(=O)C(C)C]

In some embodiments, R^{D2A} is

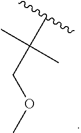 [SMILES: *C(C)(C)COC]

In some embodiments, R^{D2A} is

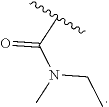 [SMILES: *C(=O)N(C)CC]

In some embodiments, R^{D2A} is

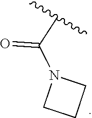 [SMILES: *C(=O)N1CCC1]

In some embodiments, R^{D2A} is

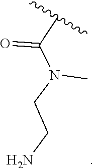 [SMILES: *C(=O)N(C)CCN]

In some embodiments, R^{D2A} is

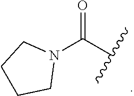 [SMILES: *C(=O)N1CCCC1]

In some embodiments, R^{D2A} is

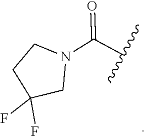 [SMILES: *C(=O)N1CCC(F)(F)C1]

In some embodiments, R^{D2A} is

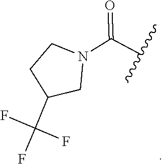 [SMILES: *C(=O)N1CCC(C(F)(F)F)C1]

In some embodiments, R^{D2A} and R^{D1} are taken together with their intervening atoms to form

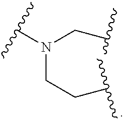 [SMILES: *CCN(*)C*]

In some embodiments, R^{D2A} and R^{D1} are taken together with their intervening atoms to form

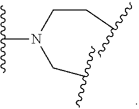 [SMILES: *CCN(*)C*]

In some embodiments, R^{D2A} and R^{D1} are taken together with their intervening atoms to form

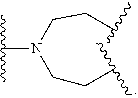 [SMILES: *CCN(*)CC*]

In some embodiments, R^{D2A} and R^{D1}, together with ^{RD2}, are taken together with their

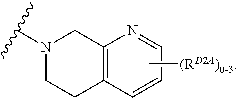

In some embodiments, R^{D2A} and R^{D1} intervening atoms to form together with R^{D2}, are taken together with their intervening atoms to form

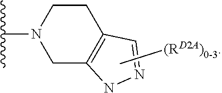

In some embodiments, R^{D2A} and R^{D1}, together with ^{RD2}, are taken together with their intervening atoms to form

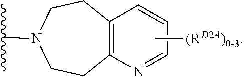

In some embodiments, R^{D2A} is selected from the groups depicted in the compounds in Table 1

As defined generally above, each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, R^{A} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, R^{A} is selected from the groups depicted in the compounds in Table 1.

As defined generally above, each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{B} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{B} is selected from the groups depicted in the compounds in Table 1.

As defined generally above, each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R^{C} is oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R^{C} is selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur. In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur. In some embodiments, R is selected from the groups depicted in the compounds in Table 1.

In some embodiments, the present disclosure provides a compound of formula I or I′ wherein Cy^{A} is

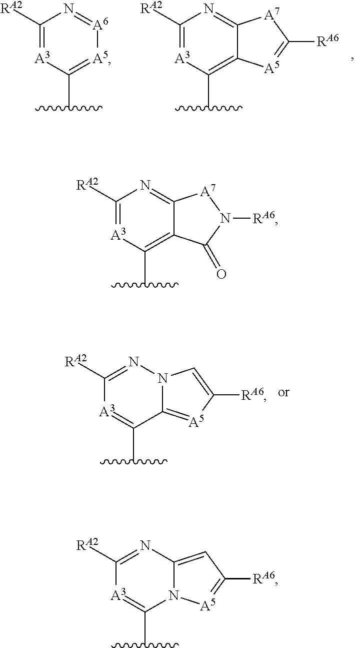

thereby forming a compound of one of formulas II-a, II-b, II-c, II-d, or II-e, respectively:

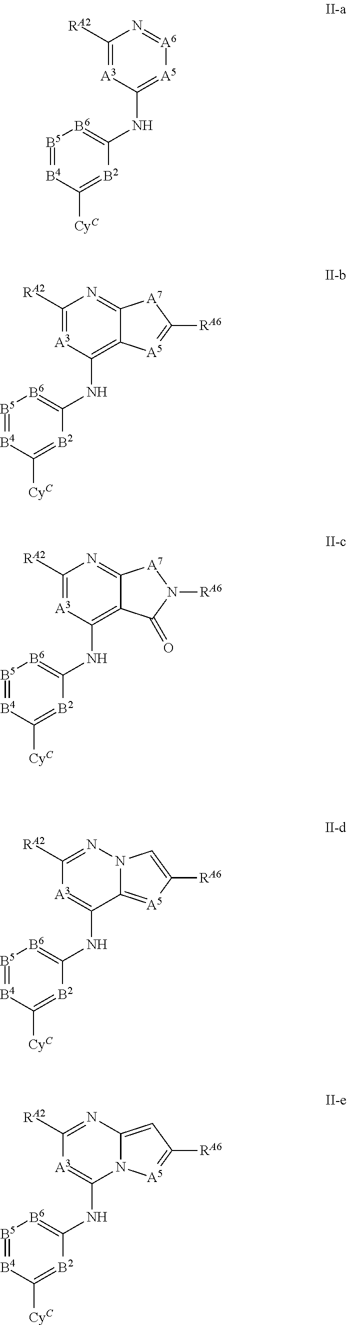

or a pharmaceutically acceptable salt thereof, wherein each of A³, A⁵, A⁶, A⁷, R^{A2}, R^{A6}, B², B⁴, B⁵, B⁶, and Cy^{C} is as defined in embodiments and classes and subclasses herein.

In some embodiments, the present disclosure provides a compound of one of formulas II-a, II-b, II-c, II-d, or II-e wherein R^{C1} is azetidin-3-yl, thereby forming a compound of one of formulas III-a, III-b, III-c, III-d, or III-e, respectively:

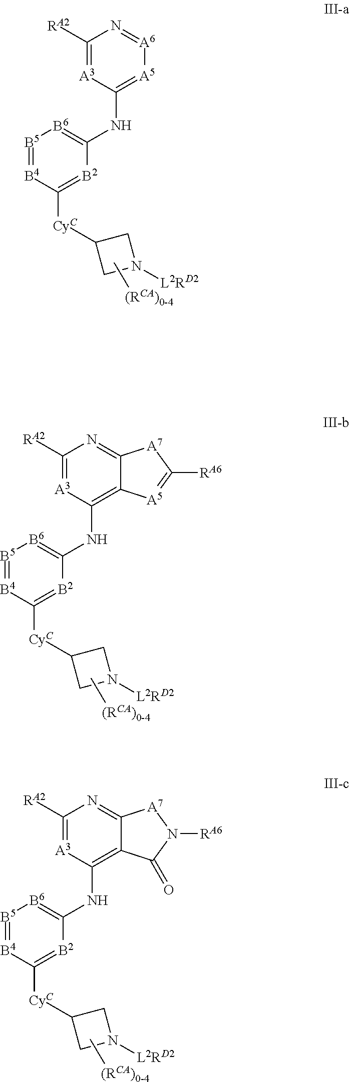

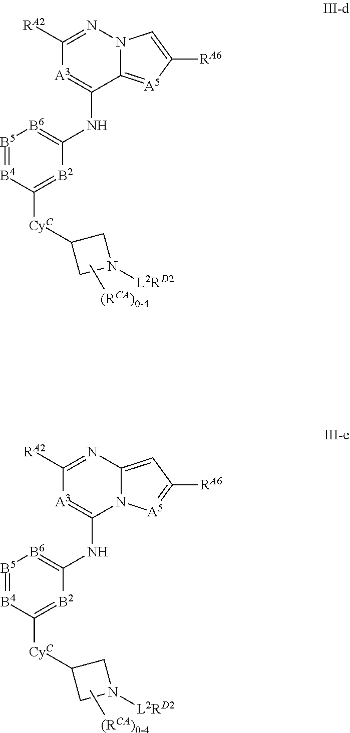

or a pharmaceutically acceptable salt thereof, wherein each of A³, A³, A⁶, A⁷, R^{A2}, R^{A5}, R^{CA}, R^{D2}, B², B⁴, B⁵, B⁶, and L² is as defined in embodiments and classes and subclasses herein.

In some embodiments, the present disclosure provides a compound of one of formulas

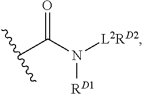

II-a, II-b, II-c, II-d, or II-e wherein R^{C1} is thereby forming a compound of one of formulas IV-a, IV-b, IV-c, IV-d, or IV-e, respectively:

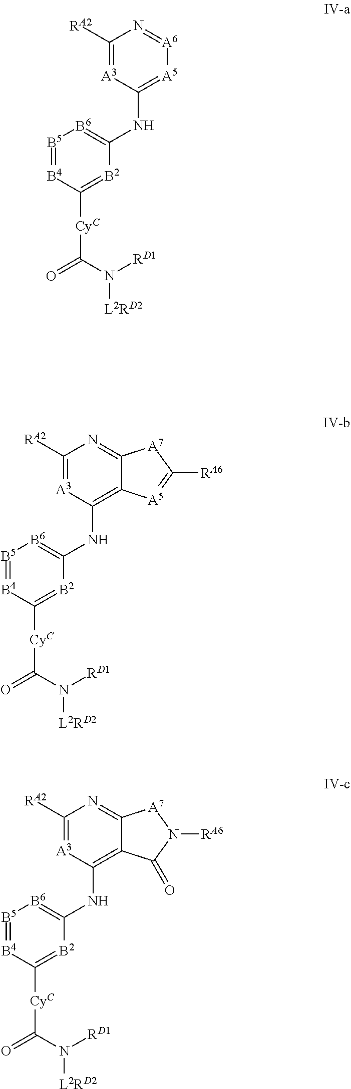

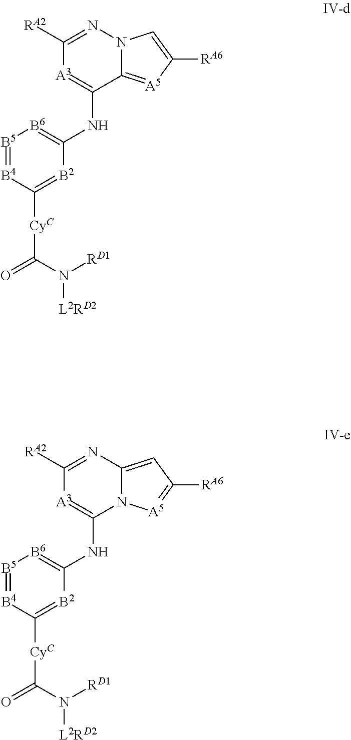

or a pharmaceutically acceptable salt thereof, wherein each of A³, A⁵, A⁶, A⁷, R^{A2}, R^{A6}, R^{D1}, R^{D2}, B², B⁴, B⁵, B⁶, and L² is as defined in embodiments and classes and subclasses herein.

In some embodiments, the present disclosure provides a compound of one of formulas I, II-a, II-b, II-c, II-d, II-e, III-a, III-b, III-c, III-d, III-e, IV-a, IV-b, IV-c, IV-d, or IV-e, or a pharmaceutically acceptable salt thereof, wherein B² is C(R^{B2}).

In some embodiments, the present disclosure provides a compound of one of formulas I, II-a, II-b, II-c, II-d, II-e, III-a, III-b, III-c, III-d, III-e, IV-a, IV-b, IV-c, IV-d, or IV-e, or a pharmaceutically acceptable salt thereof, wherein B⁴ is CH.

In some embodiments, the present disclosure provides a compound of one of formulas I, II-a, II-b, II-c, II-d, II-e, III-a, III-b, III-c, III-d, III-e, IV-a, IV-b, IV-c, IV-d, or IV-e, or a pharmaceutically acceptable salt thereof, wherein B⁴ is C(R^{B4}).

In some embodiments, the present disclosure provides a compound of one of formulas I, II-a, II-b, II-c, II-d, II-e, III-a, III-b, III-c, III-d, III-e, IV-a, IV-b, IV-c, IV-d, or IV-e, or a pharmaceutically acceptable salt thereof, wherein B⁵ is CH.

In some embodiments, the present disclosure provides a compound of one of formulas I, II-a, II-b, II-c, II-d, II-e, III-a, III-b, III-c, III-d, III-e, IV-a, IV-b, IV-c, IV-d, or IV-e, or a pharmaceutically acceptable salt thereof, wherein B⁵ is C(R^{B5}).

In some embodiments, the present disclosure provides a compound of one of formulas I, II-a, II-b, II-c, II-d, II-e, III-a, III-b, III-c, III-d, III-e, IV-a, IV-b, IV-c, IV-d, or IV-e, or a pharmaceutically acceptable salt thereof, wherein R^{B2} is-OR.

In some embodiments, the present disclosure provides a compound of one of formulas I, II-a, II-b, II-c, II-d, II-e, III-a, III-b, III-c, III-d, III-e, IV-a, IV-b, IV-c, IV-d, or IV-e, or a pharmaceutically acceptable salt thereof, wherein Cy^{C} is pyrazole, triazole, thiazole, oxazole, thiadiazole, oxadiazole, phenyl, pyridine, pyrimidine, pyrazine, or pyridazine.

In some embodiments, the compound is not a compound disclosed in CN application Ser. No. 11190949140 or U.S. Pat. No. 10,000,480.

Examples of compounds of the present disclosure include those listed in the Tables and exemplification herein, or a pharmaceutically acceptable salt, stereoisomer, or mixture of stereoisomers thereof. In some embodiments, the present disclosure provides a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

TABLE 1
Representative Compounds of the Disclosure with Bioactivity Data.

| | | | | Cell PD- | |
|---|---|---|---|---|---|
| | | | | TF1 | Cell PD |
| | | SPR | SPR | JAK2 | TF1 |
| Com- | | JAK2 | JAK2 | V617F | V617F |
| pound | | JH2 | JH1 | STAT3 | STAT5 |
| No. | Structure | KD | KD | (IC₅₀) | (IC₅₀) |
| 1 | 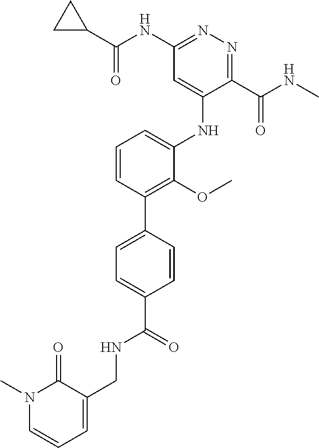 | A | D | D | D |
| 2 | 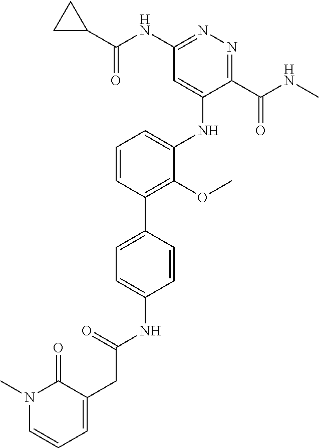 | A | | E | E |
| 3 | 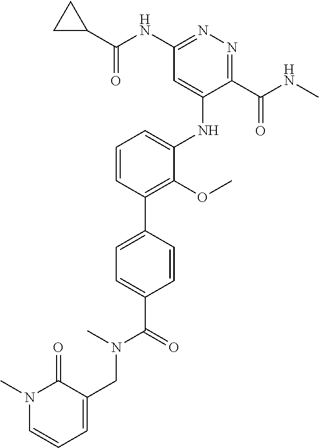 | A | E | D | D |
| 4 | 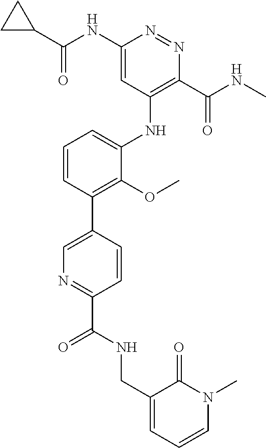 [SMILES: CNC(=O)c1nnc(NC(=O)C2CC2)cc1Nc1cccc(-c2ccc(C(=O)NCc3cccn(C)c3=O)nc2)c1OC] | A | E | D | D |
| 5 | 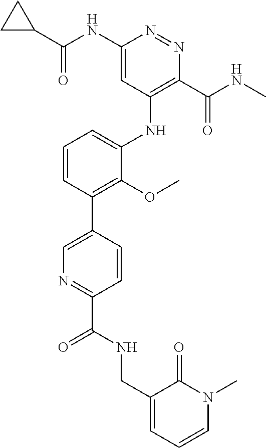 [SMILES: CNC(=O)c1nnc(NC(=O)C2CC2)cc1Nc1cccc(-c2ccc(C(=O)NCc3cccn(C)c3=O)nc2)c1OC] | A | E | D | D |
| 6 | 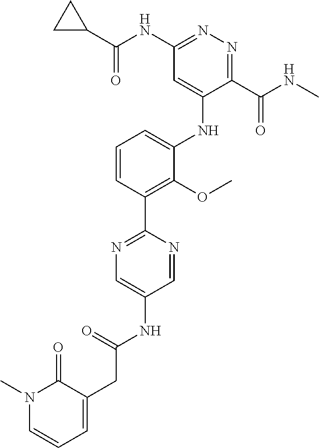 | A | D | E | E |
| 7 | 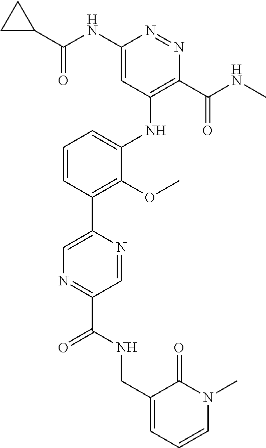 [SMILES: CNC(=O)c1nnc(NC(=O)C2CC2)cc1Nc1cccc(-c2cnc(C(=O)NCc3cccn(C)c3=O)cn2)c1OC] | A | | D | E |
| 8 | 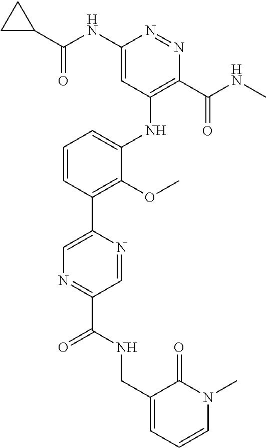 [SMILES: CNC(=O)c1nnc(NC(=O)C2CC2)cc1Nc1cccc(-c2cnc(C(=O)NCc3cccn(C)c3=O)cn2)c1OC] | A | E | D | E |
| 9 | 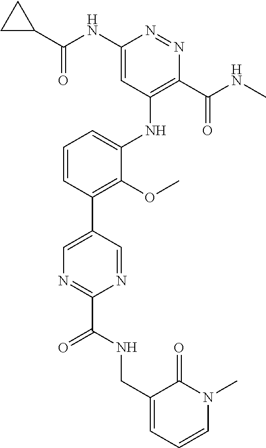 [SMILES: CNC(=O)c1nnc(NC(=O)C2CC2)cc1Nc1cccc(-c2cnc(C(=O)NCc3cccn(C)c3=O)nc2)c1OC] | A | D | D | E |
| 10 | 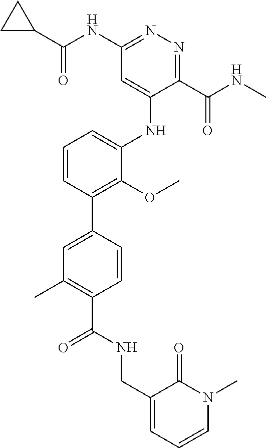 [SMILES: CNC(=O)c1nnc(NC(=O)C2CC2)cc1Nc1cccc(-c2ccc(C(=O)NCc3cccn(C)c3=O)c(C)c2)c1OC] | A | E | D | D |
| 11 | 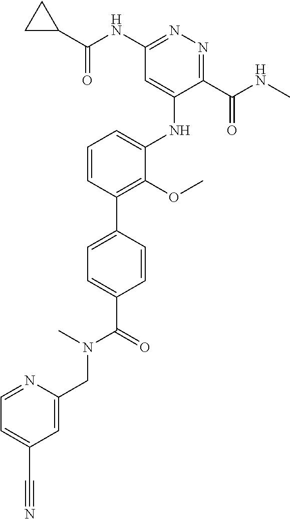 [SMILES: CNC(=O)c1nnc(NC(=O)C2CC2)cc1Nc1cccc(-c2ccc(C(=O)N(C)Cc3cc(C#N)ccn3)cc2)c1OC] | A | E | D | D |
| 12 | 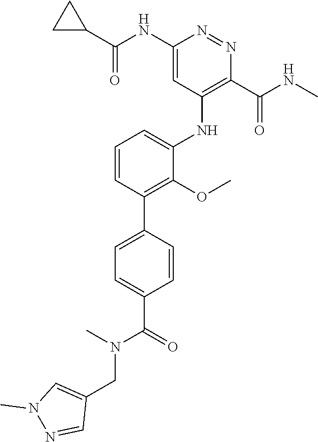 | A | | D | D |
| 13 | 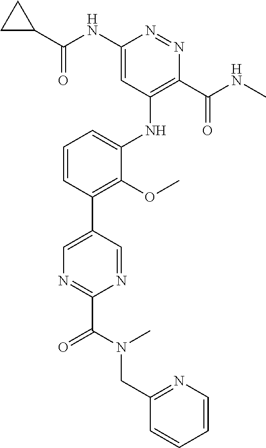 [SMILES: CNC(=O)c1nnc(NC(=O)C2CC2)cc1Nc1cccc(-c2cnc(C(=O)N(C)Cc3ccccn3)nc2)c1OC] | A | | D | D |
| 14 | 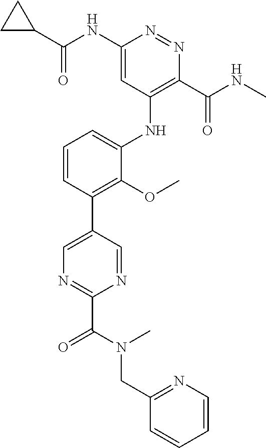 [SMILES: CNC(=O)c1nnc(NC(=O)C2CC2)cc1Nc1cccc(-c2cnc(C(=O)N(C)Cc3ccccn3)nc2)c1OC] | A | | | |
| 15 | 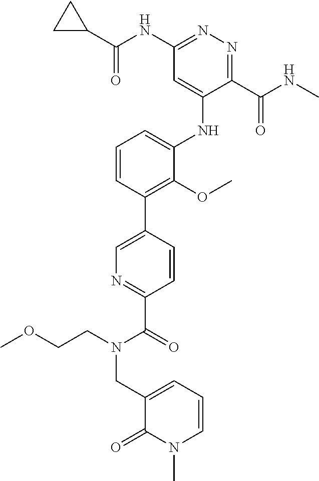 | A | | E | E |
| 16 | 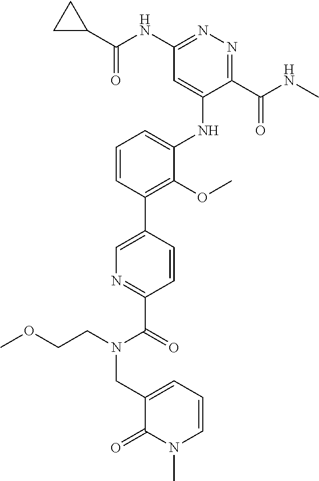 | A | | D | E |
| 17 | 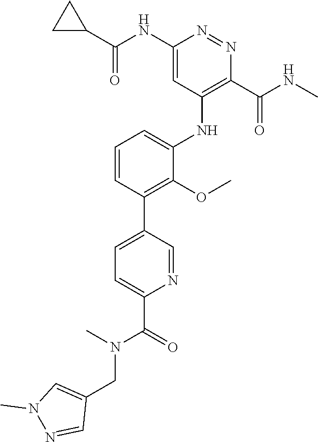 | A | E | C | C |
| 18 | 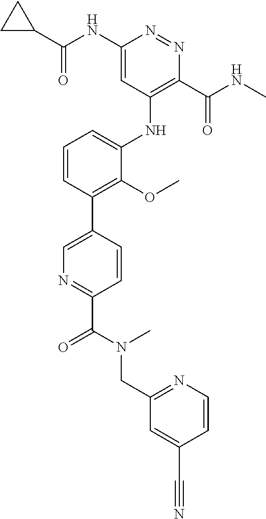 [SMILES: CNC(=O)c1nnc(NC(=O)C2CC2)cc1Nc1cccc(-c2ccc(C(=O)N(C)Cc3cc(C#N)ccn3)nc2)c1OC] | A | E | C | C |
| 19 | 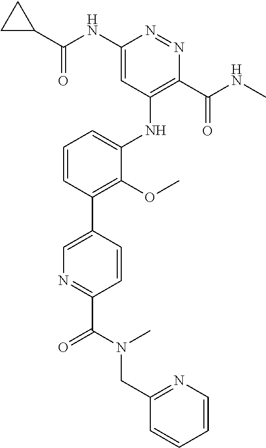 [SMILES: CNC(=O)c1nnc(NC(=O)C2CC2)cc1Nc1cccc(-c2ccc(C(=O)N(C)Cc3ccccn3)nc2)c1OC] | A | | D | D |
| 20 | 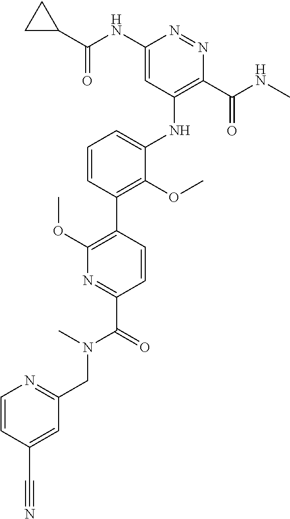 [SMILES: CNC(=O)c1nnc(NC(=O)C2CC2)cc1Nc1cccc(-c2ccc(C(=O)N(C)Cc3cc(C#N)ccn3)nc2OC)c1OC] | A | | D | D |
| 21 | 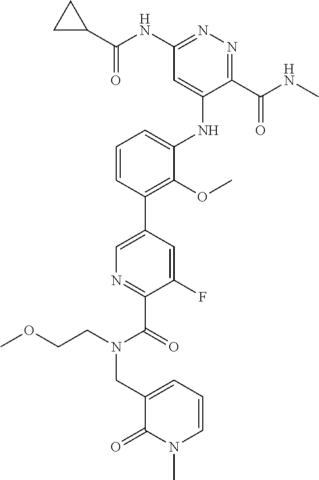 | A | | D | D |
| 22 | 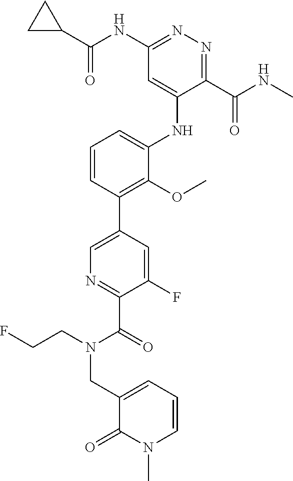 [SMILES: CNC(=O)c1nnc(NC(=O)C2CC2)cc1Nc1cccc(-c2cnc(C(=O)N(CCF)Cc3cccn(C)c3=O)c(F)c2)c1OC] | A | | D | D |
| 23 | 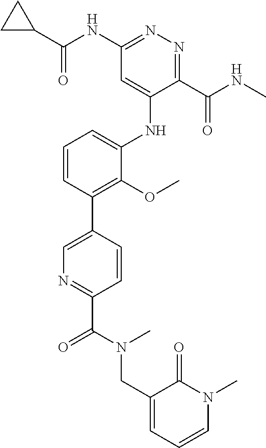 [SMILES: CNC(=O)c1nnc(NC(=O)C2CC2)cc1Nc1cccc(-c2ccc(C(=O)N(C)Cc3cccn(C)c3=O)nc2)c1OC] | A | | D | C |
| 24 | 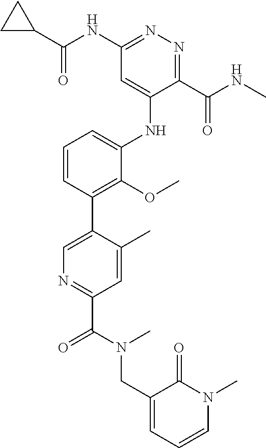 [SMILES: CNC(=O)c1nnc(NC(=O)C2CC2)cc1Nc1cccc(-c2cnc(C(=O)N(C)Cc3cccn(C)c3=O)cc2C)c1OC] | A | | D | D |
| 25 | 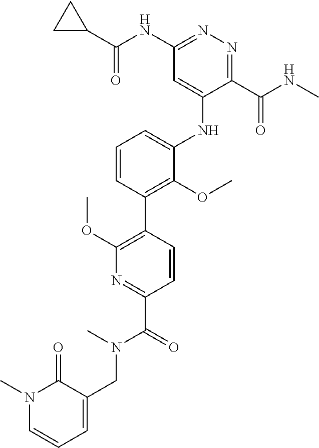 | A | | D | D |
| 26 | 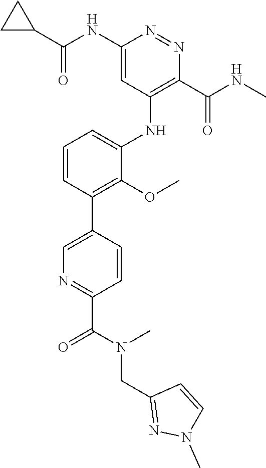 [SMILES: CNC(=O)c1nnc(NC(=O)C2CC2)cc1Nc1cccc(-c2ccc(C(=O)N(C)Cc3ccn(C)n3)nc2)c1OC] | A | E | C | C |
| 27 | 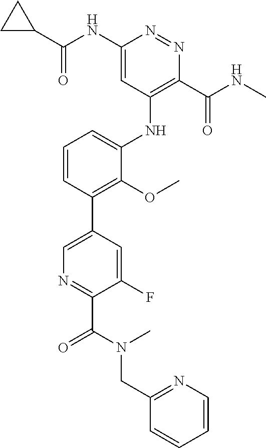 [SMILES: CNC(=O)c1nnc(NC(=O)C2CC2)cc1Nc1cccc(-c2cnc(C(=O)N(C)Cc3ccccn3)c(F)c2)c1OC] | A | | C | C |
| 28 | 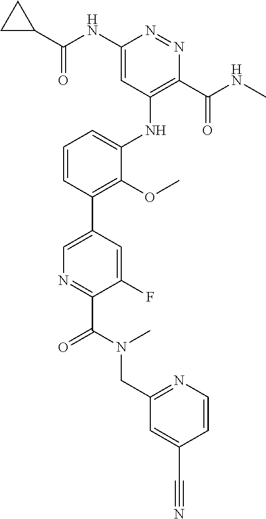 [SMILES: CNC(=O)c1nnc(NC(=O)C2CC2)cc1Nc1cccc(-c2cnc(C(=O)N(C)Cc3cc(C#N)ccn3)c(F)c2)c1OC] | A | E | C | C |
| 29 | 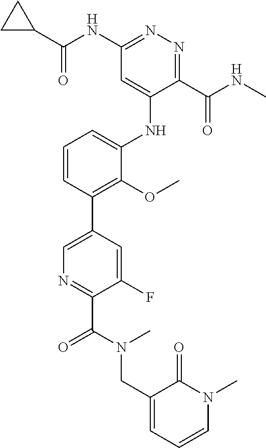 [SMILES: CNC(=O)c1nnc(NC(=O)C2CC2)cc1Nc1cccc(-c2cnc(C(=O)N(C)Cc3cccn(C)c3=O)c(F)c2)c1OC] | A | | C | C |
| 30 | 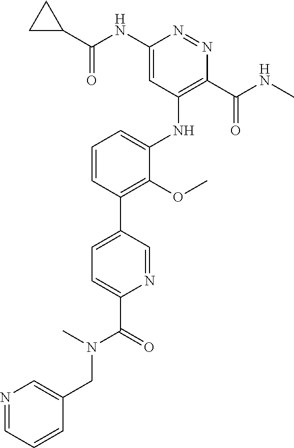 [SMILES: CNC(=O)c1nnc(NC(=O)C2CC2)cc1Nc1cccc(-c2ccc(C(=O)N(C)Cc3cccnc3)nc2)c1OC] | A | E | C | C |
| 31 | 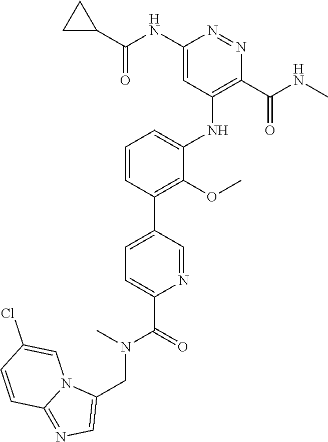 [SMILES: CNC(=O)c1nnc(NC(=O)C2CC2)cc1Nc1cccc(-c2ccc(C(=O)N(C)Cc3cnc4ccc(Cl)cn34)nc2)c1OC] | A | | D | D |
| 32 | 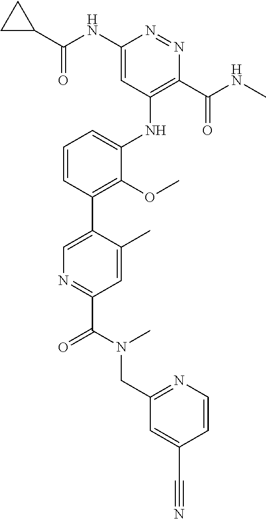 [SMILES: CNC(=O)c1nnc(NC(=O)C2CC2)cc1Nc1cccc(-c2cnc(C(=O)N(C)Cc3cc(C#N)ccn3)cc2C)c1OC] | A | E | D | D |
| 33 | 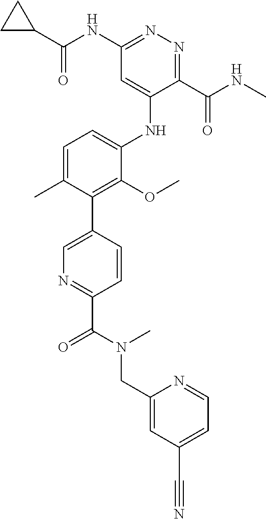 [SMILES: CNC(=O)c1nnc(NC(=O)C2CC2)cc1Nc1ccc(C)c(-c2ccc(C(=O)N(C)Cc3cc(C#N)ccn3)nc2)c1OC] | A | E | B | B |
| 34 | 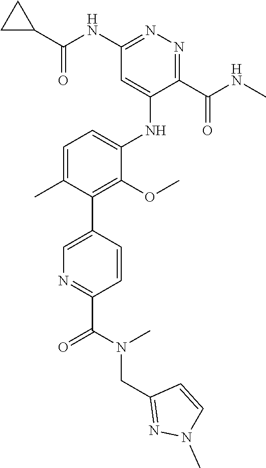 [SMILES: CNC(=O)c1nnc(NC(=O)C2CC2)cc1Nc1ccc(C)c(-c2ccc(C(=O)N(C)Cc3ccn(C)n3)nc2)c1OC] | A | E | C | C |
| 35 | 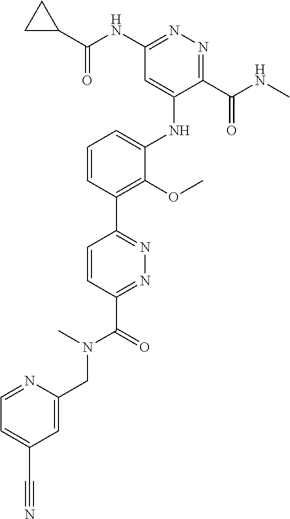 [SMILES: CNC(=O)c1nnc(NC(=O)C2CC2)cc1Nc1cccc(-c2ccc(C(=O)N(C)Cc3cc(C#N)ccn3)nn2)c1OC] | A | E | C | D |
| 36 | 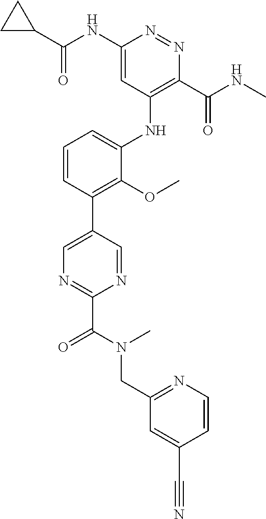 [SMILES: CNC(=O)c1nnc(NC(=O)C2CC2)cc1Nc1cccc(-c2cnc(C(=O)N(C)Cc3cc(C#N)ccn3)nc2)c1OC] | A | E | C | C |
| 37 | 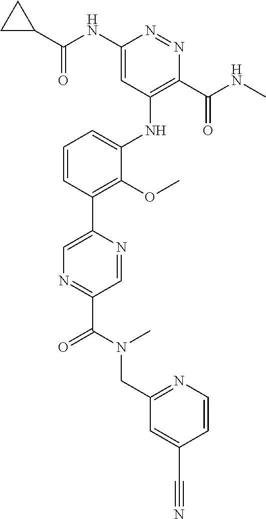 [SMILES: CNC(=O)c1nnc(NC(=O)C2CC2)cc1Nc1cccc(-c2cnc(C(=O)N(C)Cc3cc(C#N)ccn3)cn2)c1OC] | A | E | C | C |
| 38 | 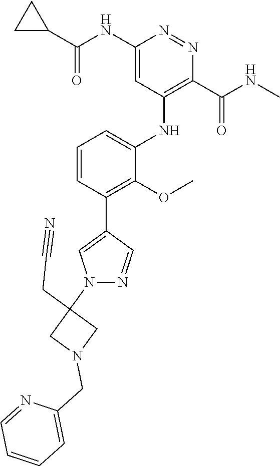 [SMILES: CNC(=O)c1nnc(NC(=O)C2CC2)cc1Nc1cccc(-c2cnn(C3(CC#N)CN(Cc4ccccn4)C3)c2)c1OC] | B | | D | E |
| 39 | 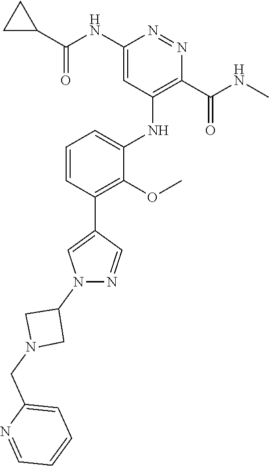 [SMILES: CNC(=O)c1nnc(NC(=O)C2CC2)cc1Nc1cccc(-c2cnn(C3CN(Cc4ccccn4)C3)c2)c1OC] | A | D | C | C |
| 40 | 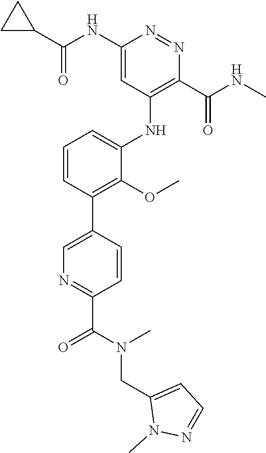 [SMILES: CNC(=O)c1nnc(NC(=O)C2CC2)cc1Nc1cccc(-c2ccc(C(=O)N(C)Cc3ccnn3C)nc2)c1OC] | A | E | D | D |
| 41 | 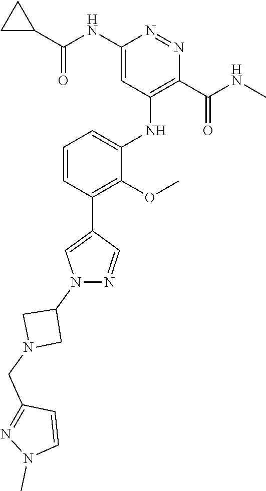 [SMILES: CNC(=O)c1nnc(NC(=O)C2CC2)cc1Nc1cccc(-c2cnn(C3CN(Cc4ccn(C)n4)C3)c2)c1OC] | A | D | C | C |
| 42 | 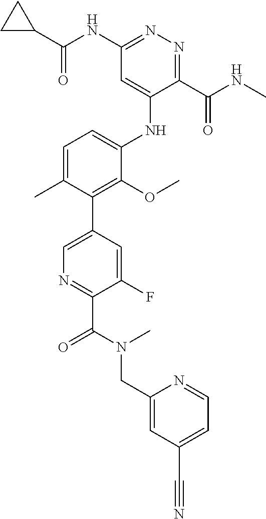 [SMILES: CNC(=O)c1nnc(NC(=O)C2CC2)cc1Nc1ccc(C)c(-c2cnc(C(=O)N(C)Cc3cc(C#N)ccn3)c(F)c2)c1OC] | A | E | C | C |
| 43 | 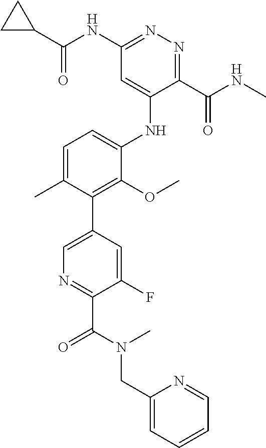 [SMILES: CNC(=O)c1nnc(NC(=O)C2CC2)cc1Nc1ccc(C)c(-c2cnc(C(=O)N(C)Cc3ccccn3)c(F)c2)c1OC] | A | E | D | D |
| 44 | 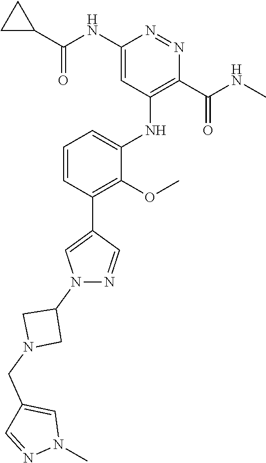 [SMILES: CNC(=O)c1nnc(NC(=O)C2CC2)cc1Nc1cccc(-c2cnn(C3CN(Cc4cnn(C)c4)C3)c2)c1OC] | A | D | D | D |
| 45 | 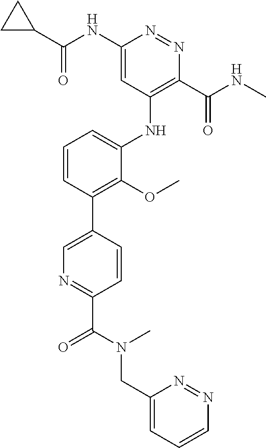 [SMILES: CNC(=O)c1nnc(NC(=O)C2CC2)cc1Nc1cccc(-c2ccc(C(=O)N(C)Cc3cccnn3)nc2)c1OC] | A | | D | D |
| 46 | 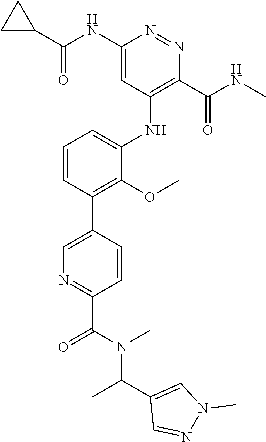 [SMILES: CNC(=O)c1nnc(NC(=O)C2CC2)cc1Nc1cccc(-c2ccc(C(=O)N(C)C(C)c3cnn(C)c3)nc2)c1OC] | A | | | |
| 47 | 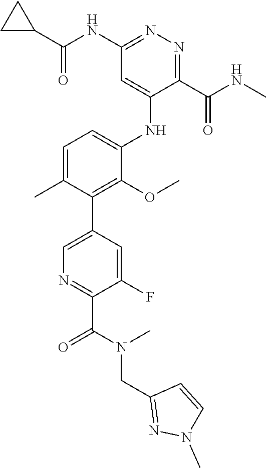 [SMILES: CNC(=O)c1nnc(NC(=O)C2CC2)cc1Nc1ccc(C)c(-c2cnc(C(=O)N(C)Cc3ccn(C)n3)c(F)c2)c1OC] | A | | D | D |
| 48 | 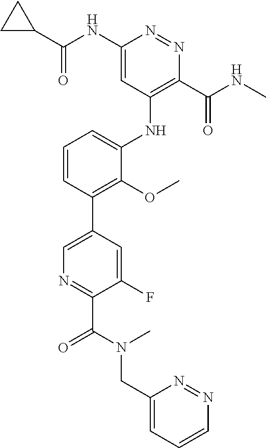 [SMILES: CNC(=O)c1nnc(NC(=O)C2CC2)cc1Nc1cccc(-c2cnc(C(=O)N(C)Cc3cccnn3)c(F)c2)c1OC] | A | E | D | D |
| 49 | 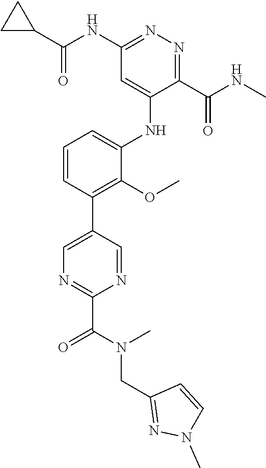 [SMILES: CNC(=O)c1nnc(NC(=O)C2CC2)cc1Nc1cccc(-c2cnc(C(=O)N(C)Cc3ccn(C)n3)nc2)c1OC] | A | | D | D |
| 50 | 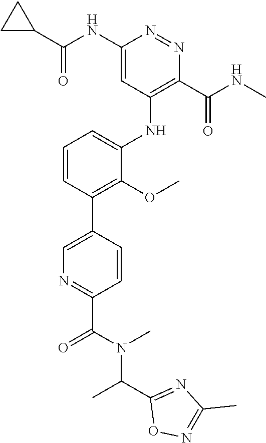 [SMILES: CNC(=O)c1nnc(NC(=O)C2CC2)cc1Nc1cccc(-c2ccc(C(=O)N(C)C(C)c3nc(C)no3)nc2)c1OC] | A | | E | E |
| 51 | 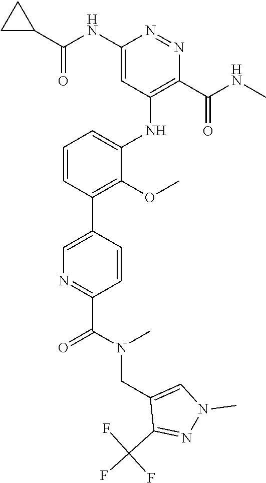 [SMILES: CNC(=O)c1nnc(NC(=O)C2CC2)cc1Nc1cccc(-c2ccc(C(=O)N(C)Cc3cn(C)nc3C(F)(F)F)nc2)c1OC] | A | | C | C |
| 52 | 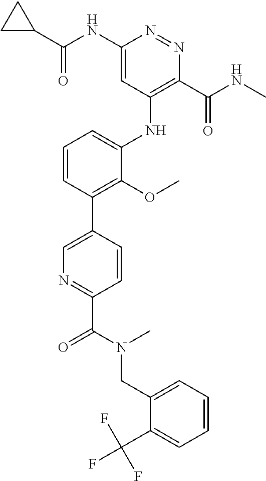 [SMILES: CNC(=O)c1nnc(NC(=O)C2CC2)cc1Nc1cccc(-c2ccc(C(=O)N(C)Cc3ccccc3C(F)(F)F)nc2)c1OC] | A | | D | D |
| 53 | 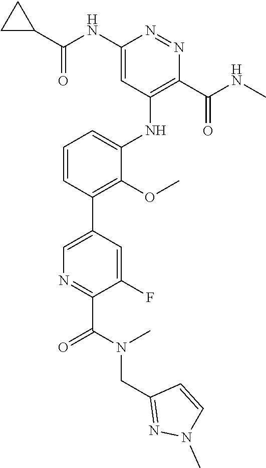 [SMILES: CNC(=O)c1nnc(NC(=O)C2CC2)cc1Nc1cccc(-c2cnc(C(=O)N(C)Cc3ccn(C)n3)c(F)c2)c1OC] | A | | D | D |
| 54 | 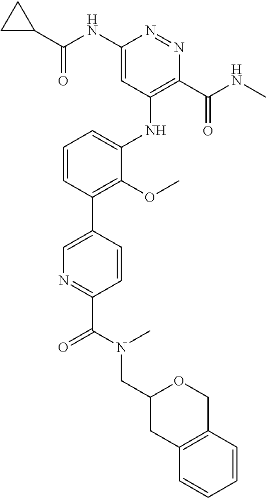 [SMILES: CNC(=O)c1nnc(NC(=O)C2CC2)cc1Nc1cccc(-c2ccc(C(=O)N(C)CC3Cc4ccccc4CO3)nc2)c1OC] | A | | D | E |
| 55 | 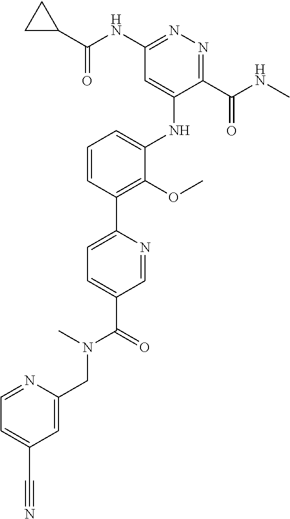 [SMILES: CNC(=O)c1nnc(NC(=O)C2CC2)cc1Nc1cccc(-c2ccc(C(=O)N(C)Cc3cc(C#N)ccn3)cn2)c1OC] | A | E | D | D |
| 56 | 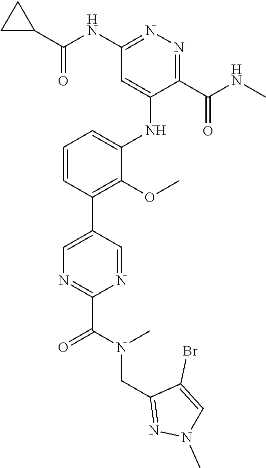 [SMILES: CNC(=O)c1nnc(NC(=O)C2CC2)cc1Nc1cccc(-c2cnc(C(=O)N(C)Cc3nn(C)cc3Br)nc2)c1OC] | A | | C | C |
| 57 | 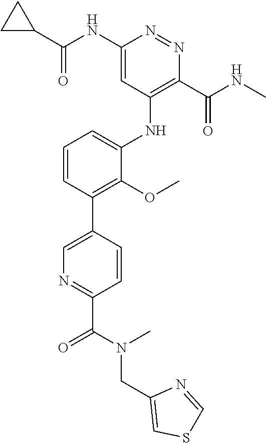 [SMILES: CNC(=O)c1nnc(NC(=O)C2CC2)cc1Nc1cccc(-c2ccc(C(=O)N(C)Cc3cscn3)nc2)c1OC] | A | | C | C |
| 58 | 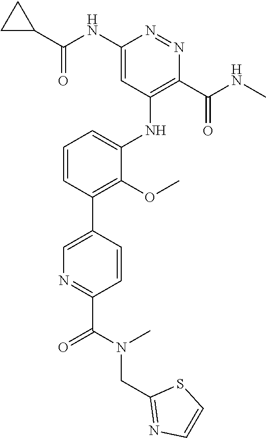 [SMILES: CNC(=O)c1nnc(NC(=O)C2CC2)cc1Nc1cccc(-c2ccc(C(=O)N(C)Cc3nccs3)nc2)c1OC] | A | | D | D |
| 59 | 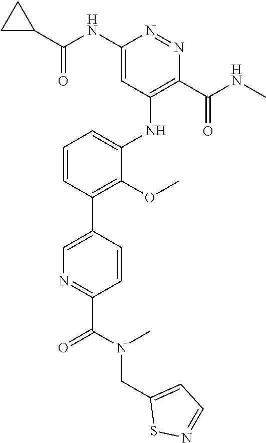 [SMILES: CNC(=O)c1nnc(NC(=O)C2CC2)cc1Nc1cccc(-c2ccc(C(=O)N(C)Cc3ccns3)nc2)c1OC] | A | | D | E |
| 60 | 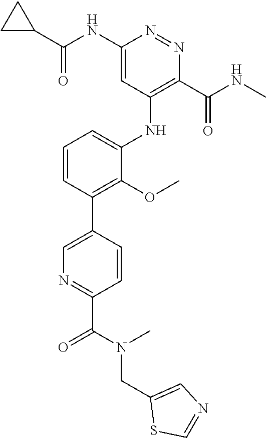 [SMILES: CNC(=O)c1nnc(NC(=O)C2CC2)cc1Nc1cccc(-c2ccc(C(=O)N(C)Cc3cncs3)nc2)c1OC] | B | | D | E |
| 61 | 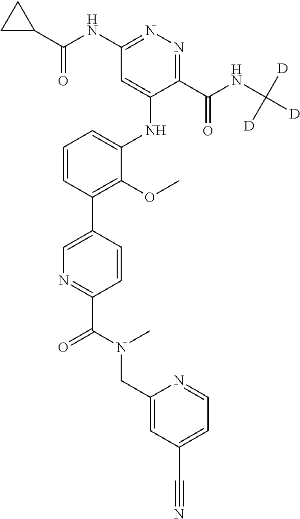 [SMILES: [2H]C([2H])([2H])NC(=O)c1nnc(NC(=O)C2CC2)cc1Nc1cccc(-c2ccc(C(=O)N(C)Cc3cc(C#N)ccn3)nc2)c1OC] | A | E | C | C |
| 62 | 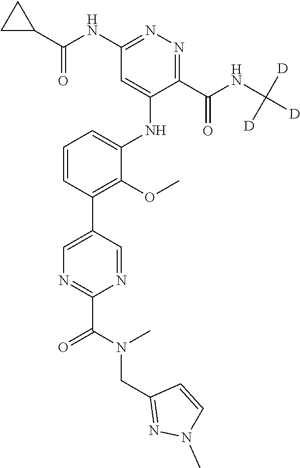 [SMILES: [2H]C([2H])([2H])NC(=O)c1nnc(NC(=O)C2CC2)cc1Nc1cccc(-c2cnc(C(=O)N(C)Cc3ccn(C)n3)nc2)c1OC] | A | | D | D |
| 63 | 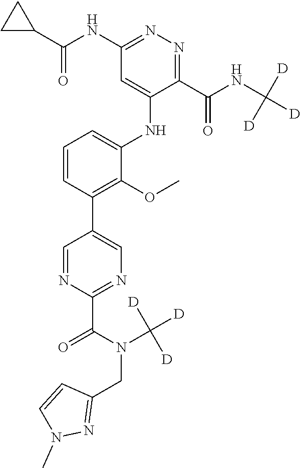 [SMILES: [2H]C([2H])([2H])NC(=O)c1nnc(NC(=O)C2CC2)cc1Nc1cccc(-c2cnc(C(=O)N(Cc3ccn(C)n3)C([2H])([2H])[2H])nc2)c1OC] | A | | D | D |
| 64 | 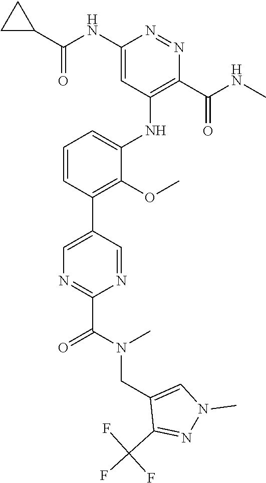 [SMILES: CNC(=O)c1nnc(NC(=O)C2CC2)cc1Nc1cccc(-c2cnc(C(=O)N(C)Cc3cn(C)nc3C(F)(F)F)nc2)c1OC] | A | | D | C |
| 65 | 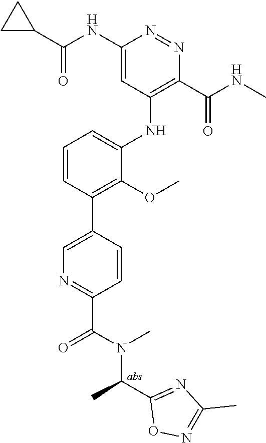 [SMILES: CNC(=O)c1nnc(NC(=O)C2CC2)cc1Nc1cccc(-c2ccc(C(=O)N(C)[C@H](C)c3nc(C)no3)nc2)c1OC] | A | | D | D |
| 66 | 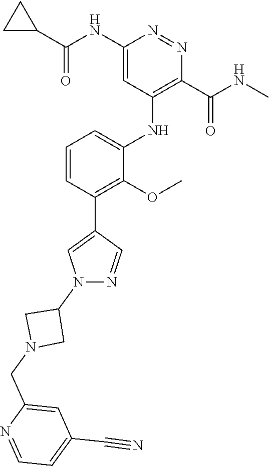 [SMILES: CNC(=O)c1nnc(NC(=O)C2CC2)cc1Nc1cccc(-c2cnn(C3CN(Cc4cc(C#N)ccn4)C3)c2)c1OC] | A | E | C | D |
| 67 | 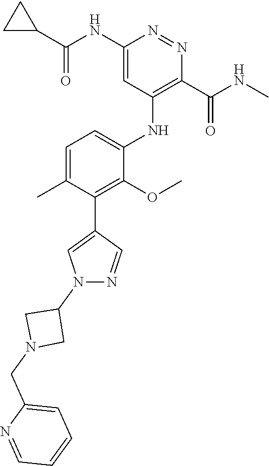 [SMILES: CNC(=O)c1nnc(NC(=O)C2CC2)cc1Nc1ccc(C)c(-c2cnn(C3CN(Cc4ccccn4)C3)c2)c1OC] | A | | D | D |
| 68 | 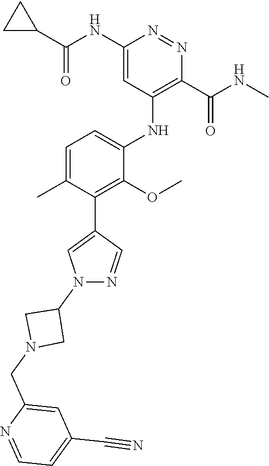 [SMILES: CNC(=O)c1nnc(NC(=O)C2CC2)cc1Nc1ccc(C)c(-c2cnn(C3CN(Cc4cc(C#N)ccn4)C3)c2)c1OC] | A | E | D | D |
| 69 | 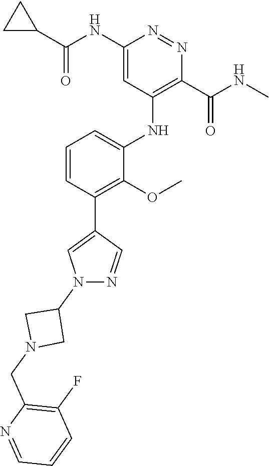 [SMILES: CNC(=O)c1nnc(NC(=O)C2CC2)cc1Nc1cccc(-c2cnn(C3CN(Cc4ncccc4F)C3)c2)c1OC] | A | | D | D |
| 70 | 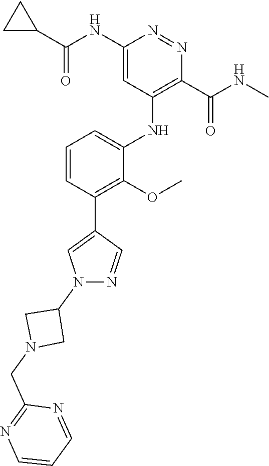 [SMILES: CNC(=O)c1nnc(NC(=O)C2CC2)cc1Nc1cccc(-c2cnn(C3CN(Cc4ncccn4)C3)c2)c1OC] | A | | D | D |
| 71 | 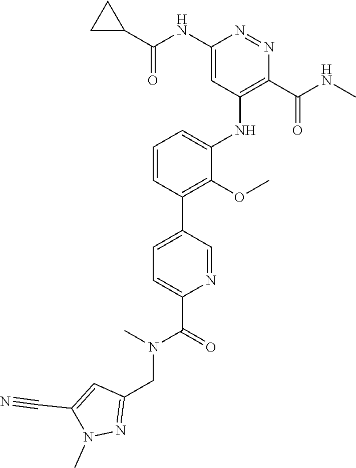 [SMILES: CNC(=O)c1nnc(NC(=O)C2CC2)cc1Nc1cccc(-c2ccc(C(=O)N(C)Cc3cc(C#N)n(C)n3)nc2)c1OC] | A | | C | C |
| 72 | 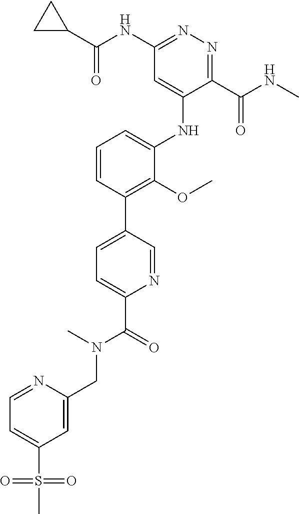 [SMILES: CNC(=O)c1nnc(NC(=O)C2CC2)cc1Nc1cccc(-c2ccc(C(=O)N(C)Cc3cc(S(C)(=O)=O)ccn3)nc2)c1OC] | A | | C | C |
| 73 | 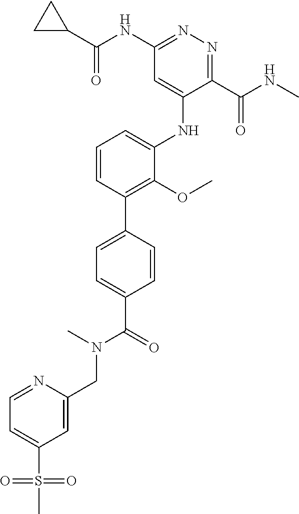 [SMILES: CNC(=O)c1nnc(NC(=O)C2CC2)cc1Nc1cccc(-c2ccc(C(=O)N(C)Cc3cc(S(C)(=O)=O)ccn3)cc2)c1OC] | A | | C | C |
| 74 | 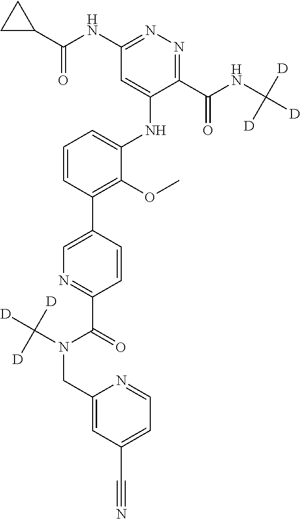 [SMILES: [2H]C([2H])([2H])NC(=O)c1nnc(NC(=O)C2CC2)cc1Nc1cccc(-c2ccc(C(=O)N(Cc3cc(C#N)ccn3)C([2H])([2H])[2H])nc2)c1OC] | A | | C | C |
| 75 | 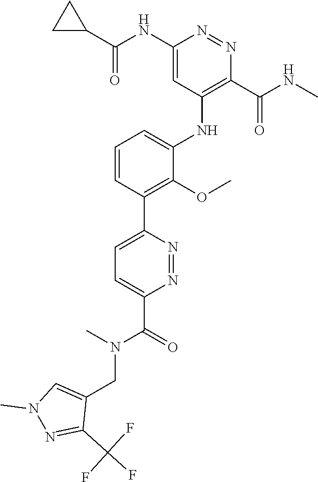 | A | | D | D |
| 76 | 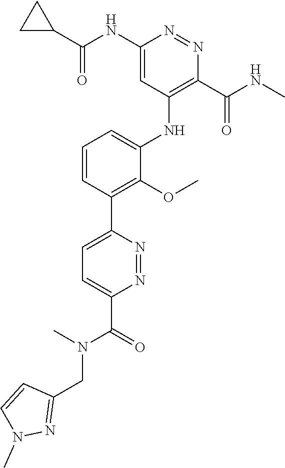 [SMILES: CNC(=O)c1nnc(NC(=O)C2CC2)cc1Nc1cccc(-c2ccc(C(=O)N(C)Cc3ccn(C)n3)nn2)c1OC] | A | | D | D |
| 77 | 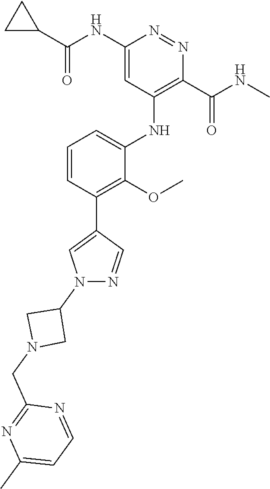 | A | | C | D |
| 78 | 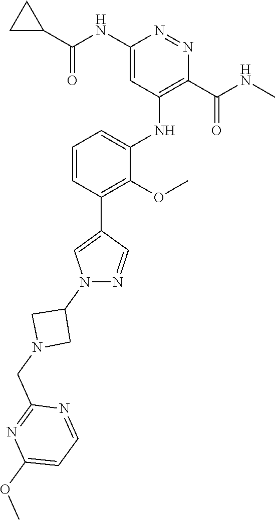 [SMILES: CNC(=O)c1nnc(NC(=O)C2CC2)cc1Nc1cccc(-c2cnn(C3CN(Cc4nccc(OC)n4)C3)c2)c1OC] | A | | D | D |
| 79 | 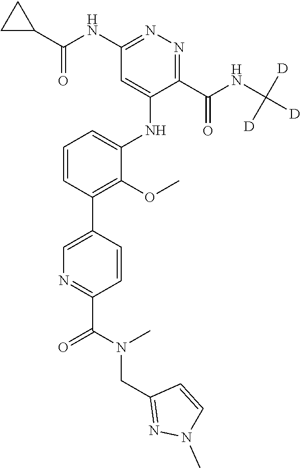 [SMILES: [2H]C([2H])([2H])NC(=O)c1nnc(NC(=O)C2CC2)cc1Nc1cccc(-c2ccc(C(=O)N(C)Cc3ccn(C)n3)nc2)c1OC] | A | | C | D |
| 80 | 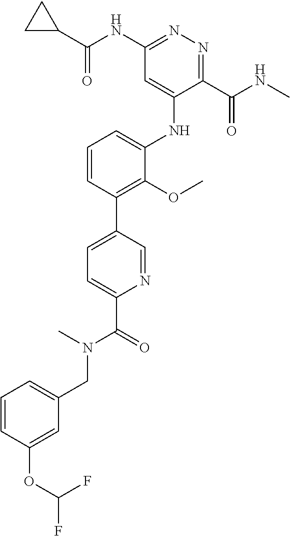 [SMILES: CNC(=O)c1nnc(NC(=O)C2CC2)cc1Nc1cccc(-c2ccc(C(=O)N(C)Cc3cccc(OC(F)F)c3)nc2)c1OC] | A | | C | D |
| 81 | 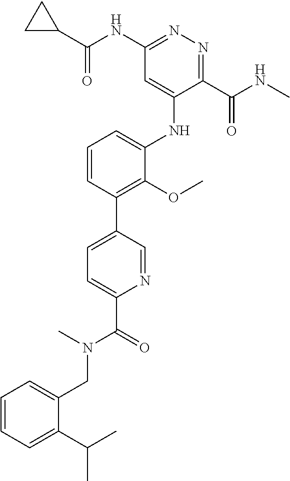 [SMILES: CNC(=O)c1nnc(NC(=O)C2CC2)cc1Nc1cccc(-c2ccc(C(=O)N(C)Cc3ccccc3C(C)C)nc2)c1OC] | B | | D | D |
| 82 | 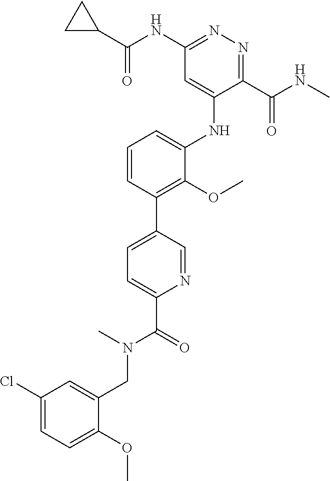 [SMILES: CNC(=O)c1nnc(NC(=O)C2CC2)cc1Nc1cccc(-c2ccc(C(=O)N(C)Cc3cc(Cl)ccc3OC)nc2)c1OC] | A | | D | D |
| 83 | 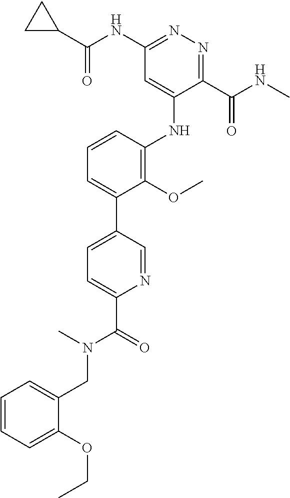 [SMILES: CCOc1ccccc1CN(C)C(=O)c1ccc(-c2cccc(Nc3cc(NC(=O)C4CC4)nnc3C(=O)NC)c2OC)cn1] | A | | D | D |
| 84 | 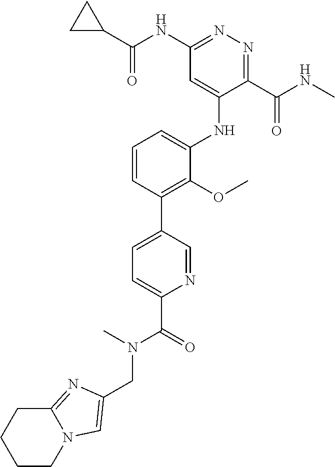 [SMILES: CNC(=O)c1nnc(NC(=O)C2CC2)cc1Nc1cccc(-c2ccc(C(=O)N(C)Cc3cn4c(n3)CCCC4)nc2)c1OC] | B | | D | E |
| 85 | 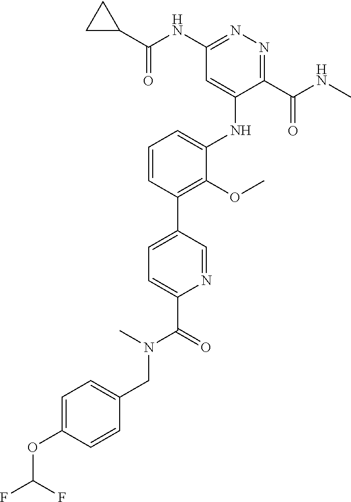 [SMILES: CNC(=O)c1nnc(NC(=O)C2CC2)cc1Nc1cccc(-c2ccc(C(=O)N(C)Cc3ccc(OC(F)F)cc3)nc2)c1OC] | A | | D | D |
| 86 | 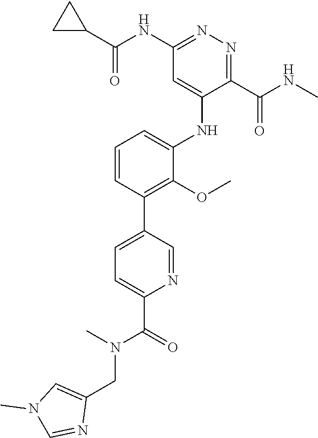 | A | | D | D |
| 87 | 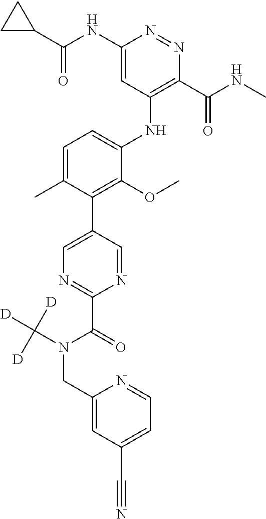 [SMILES: [2H]C([2H])([2H])N(Cc1cc(C#N)ccn1)C(=O)c1ncc(-c2c(C)ccc(Nc3cc(NC(=O)C4CC4)nnc3C(=O)NC)c2OC)cn1] | A | | | |
| 88 | 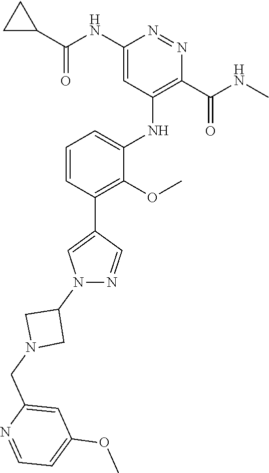 [SMILES: CNC(=O)c1nnc(NC(=O)C2CC2)cc1Nc1cccc(-c2cnn(C3CN(Cc4cc(OC)ccn4)C3)c2)c1OC] | A | | D | D |
| 89 | 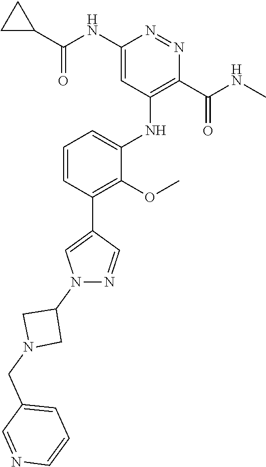 [SMILES: CNC(=O)c1nnc(NC(=O)C2CC2)cc1Nc1cccc(-c2cnn(C3CN(Cc4cccnc4)C3)c2)c1OC] | A | | C | C |
| 90 | 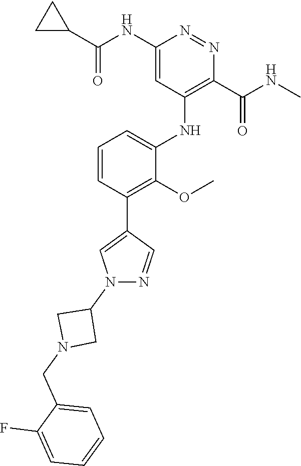 [SMILES: CNC(=O)c1nnc(NC(=O)C2CC2)cc1Nc1cccc(-c2cnn(C3CN(Cc4ccccc4F)C3)c2)c1OC] | A | | D | D |
| 91 | 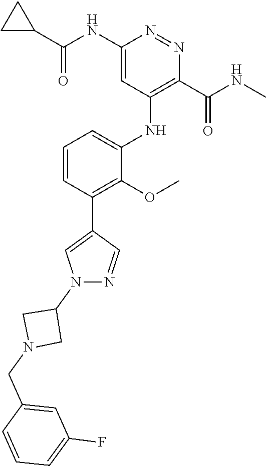 [SMILES: CNC(=O)c1nnc(NC(=O)C2CC2)cc1Nc1cccc(-c2cnn(C3CN(Cc4cccc(F)c4)C3)c2)c1OC] | A | | D | D |
| 92 | 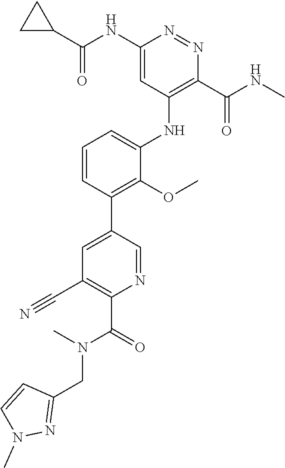 [SMILES: CNC(=O)c1nnc(NC(=O)C2CC2)cc1Nc1cccc(-c2cnc(C(=O)N(C)Cc3ccn(C)n3)c(C#N)c2)c1OC] | A | E | D | D |
| 93 | 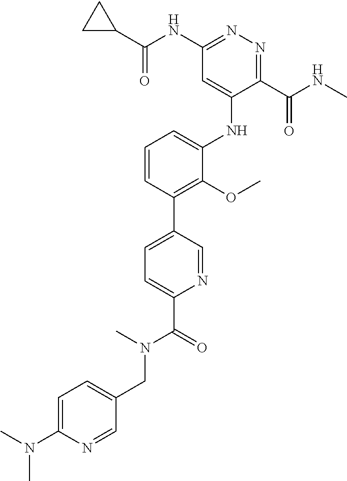 | B | | D | D |
| 94 | 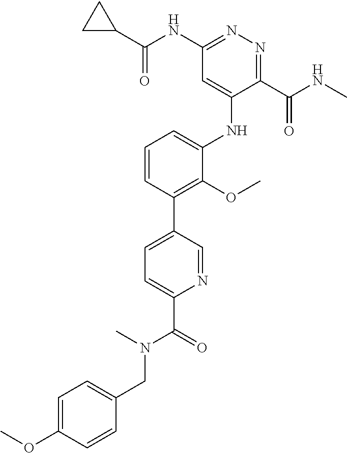 | A | | D | D |
| 95 | 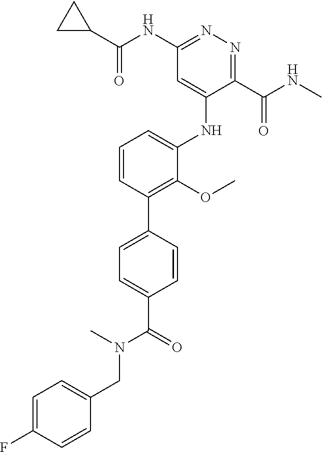 [SMILES: CNC(=O)c1nnc(NC(=O)C2CC2)cc1Nc1cccc(-c2ccc(C(=O)N(C)Cc3ccc(F)cc3)cc2)c1OC] | B | | E | E |
| 96 | 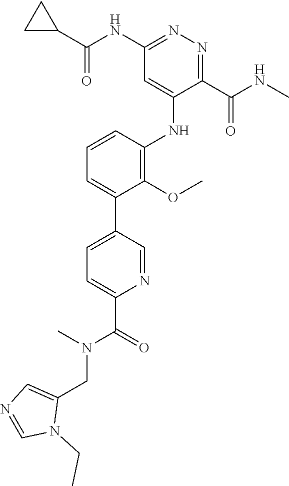 [SMILES: CCn1cncc1CN(C)C(=O)c1ccc(-c2cccc(Nc3cc(NC(=O)C4CC4)nnc3C(=O)NC)c2OC)cn1] | A | | D | D |
| 97 | 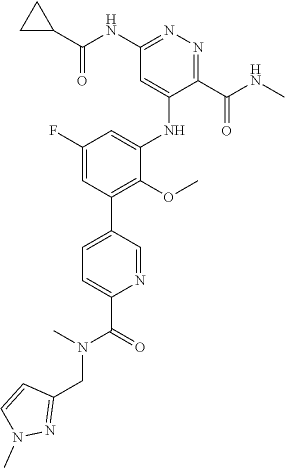 [SMILES: CNC(=O)c1nnc(NC(=O)C2CC2)cc1Nc1cc(F)cc(-c2ccc(C(=O)N(C)Cc3ccn(C)n3)nc2)c1OC] | A | | C | C |
| 98 | 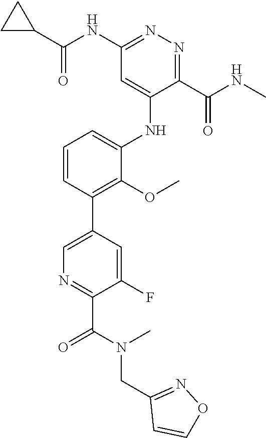 [SMILES: CNC(=O)c1nnc(NC(=O)C2CC2)cc1Nc1cccc(-c2cnc(C(=O)N(C)Cc3ccon3)c(F)c2)c1OC] | A | | D | E |
| 99 | 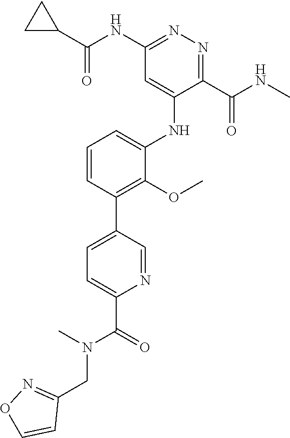 [SMILES: CNC(=O)c1nnc(NC(=O)C2CC2)cc1Nc1cccc(-c2ccc(C(=O)N(C)Cc3ccon3)nc2)c1OC] | A | | D | E |
| 100 | 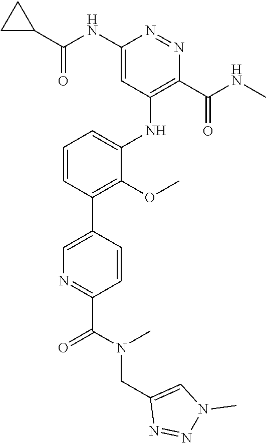 [SMILES: CNC(=O)c1nnc(NC(=O)C2CC2)cc1Nc1cccc(-c2ccc(C(=O)N(C)Cc3cn(C)nn3)nc2)c1OC] | A | | D | D |
| 101 | 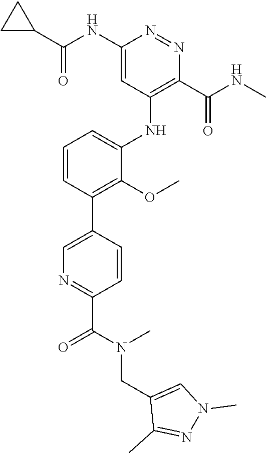 [SMILES: CNC(=O)c1nnc(NC(=O)C2CC2)cc1Nc1cccc(-c2ccc(C(=O)N(C)Cc3cn(C)nc3C)nc2)c1OC] | A | | C | C |
| 102 | 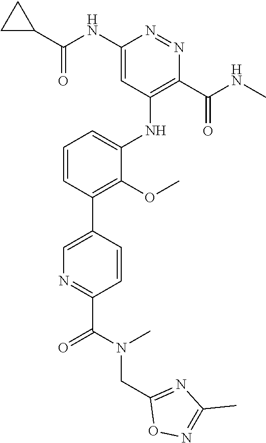 [SMILES: CNC(=O)c1nnc(NC(=O)C2CC2)cc1Nc1cccc(-c2ccc(C(=O)N(C)Cc3nc(C)no3)nc2)c1OC] | A | | D | D |
| 103 | 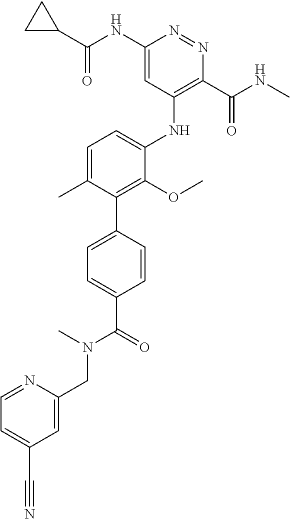 [SMILES: CNC(=O)c1nnc(NC(=O)C2CC2)cc1Nc1ccc(C)c(-c2ccc(C(=O)N(C)Cc3cc(C#N)ccn3)cc2)c1OC] | A | | C | D |
| 104 | 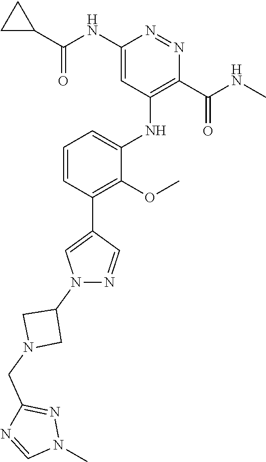 [SMILES: CNC(=O)c1nnc(NC(=O)C2CC2)cc1Nc1cccc(-c2cnn(C3CN(Cc4ncn(C)n4)C3)c2)c1OC] | A | | D | D |
| 105 | 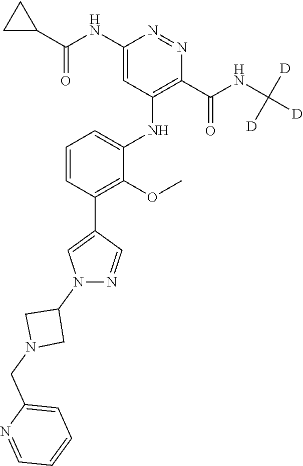 [SMILES: [2H]C([2H])([2H])NC(=O)c1nnc(NC(=O)C2CC2)cc1Nc1cccc(-c2cnn(C3CN(Cc4ccccn4)C3)c2)c1OC] | A | | C | C |
| 106 | 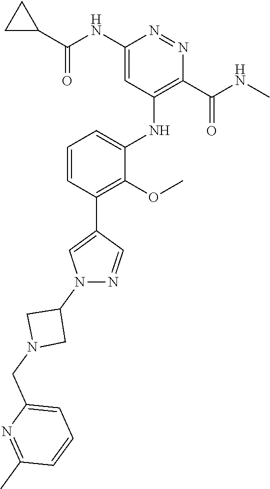 | A | | C | C |
| 107 | 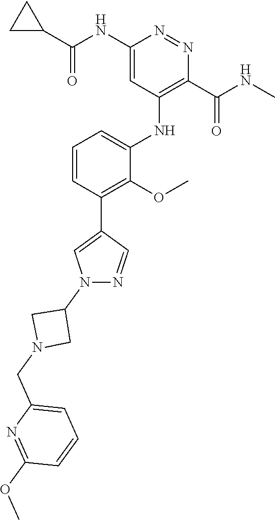 [SMILES: CNC(=O)c1nnc(NC(=O)C2CC2)cc1Nc1cccc(-c2cnn(C3CN(Cc4cccc(OC)n4)C3)c2)c1OC] | A | | C | C |
| 108 | 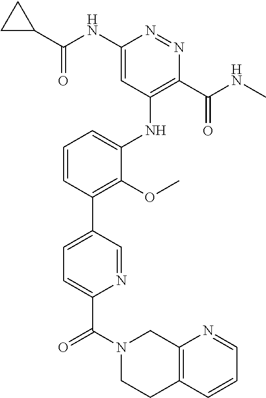 [SMILES: CNC(=O)c1nnc(NC(=O)C2CC2)cc1Nc1cccc(-c2ccc(C(=O)N3CCc4cccnc4C3)nc2)c1OC] | B | | E | E |
| 109 | 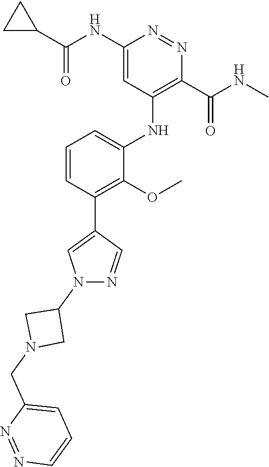 [SMILES: CNC(=O)c1nnc(NC(=O)C2CC2)cc1Nc1cccc(-c2cnn(C3CN(Cc4cccnn4)C3)c2)c1OC] | A | | C | C |
| 110 | 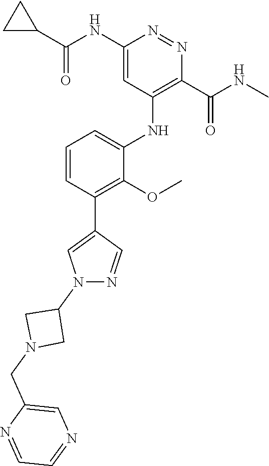 [SMILES: CNC(=O)c1nnc(NC(=O)C2CC2)cc1Nc1cccc(-c2cnn(C3CN(Cc4cnccn4)C3)c2)c1OC] | A | | C | D |
| 111 | 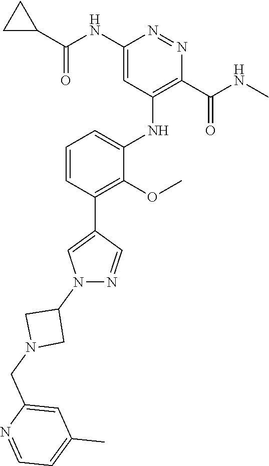 [SMILES: CNC(=O)c1nnc(NC(=O)C2CC2)cc1Nc1cccc(-c2cnn(C3CN(Cc4cc(C)ccn4)C3)c2)c1OC] | A | | C | C |
| 112 | 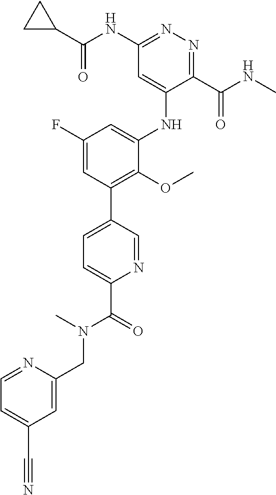 [SMILES: CNC(=O)c1nnc(NC(=O)C2CC2)cc1Nc1cc(F)cc(-c2ccc(C(=O)N(C)Cc3cc(C#N)ccn3)nc2)c1OC] | A | | C | C |
| 113 | 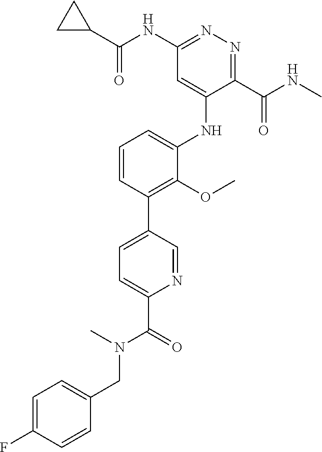 [SMILES: CNC(=O)c1nnc(NC(=O)C2CC2)cc1Nc1cccc(-c2ccc(C(=O)N(C)Cc3ccc(F)cc3)nc2)c1OC] | B | | D | D |
| 114 | 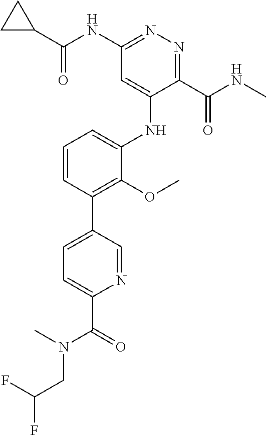 [SMILES: CNC(=O)c1nnc(NC(=O)C2CC2)cc1Nc1cccc(-c2ccc(C(=O)N(C)CC(F)F)nc2)c1OC] | B | | E | E |
| 115 | 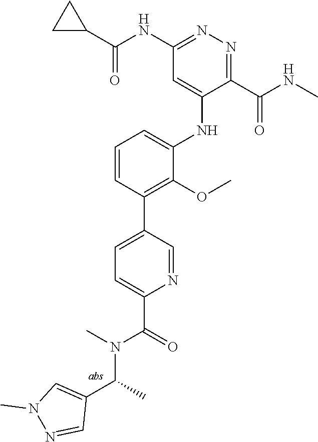 | B | | E | E |
| 116 | 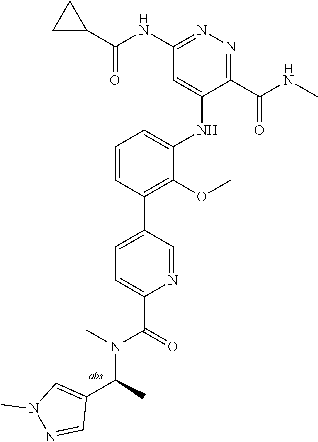 | A | | C | D |
| 117 | 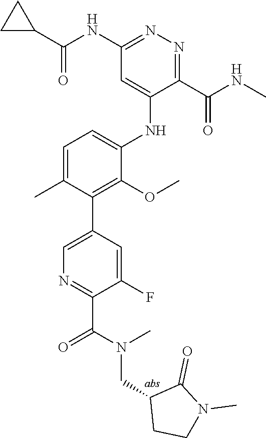 [SMILES: CNC(=O)c1nnc(NC(=O)C2CC2)cc1Nc1ccc(C)c(-c2cnc(C(=O)N(C)C[C@H]3CCN(C)C3=O)c(F)c2)c1OC] | A | | E | E |
| 118 | 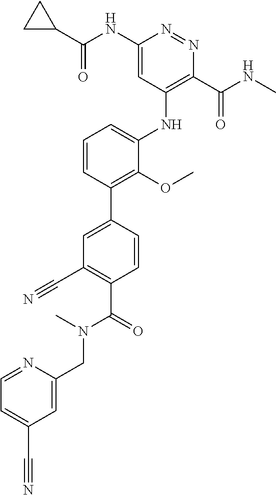 [SMILES: CNC(=O)c1nnc(NC(=O)C2CC2)cc1Nc1cccc(-c2ccc(C(=O)N(C)Cc3cc(C#N)ccn3)c(C#N)c2)c1OC] | A | | D | D |
| 119 | 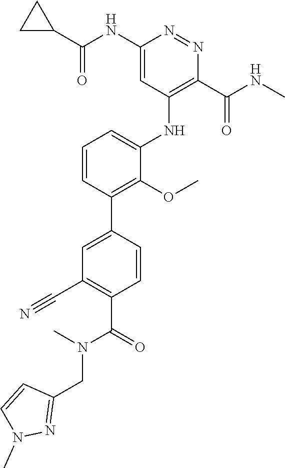 [SMILES: CNC(=O)c1nnc(NC(=O)C2CC2)cc1Nc1cccc(-c2ccc(C(=O)N(C)Cc3ccn(C)n3)c(C#N)c2)c1OC] | A | E | D | D |
| 120 | 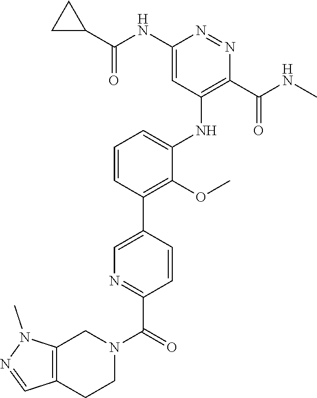 [SMILES: CNC(=O)c1nnc(NC(=O)C2CC2)cc1Nc1cccc(-c2ccc(C(=O)N3CCc4cnn(C)c4C3)nc2)c1OC] | A | | E | E |
| 121 | 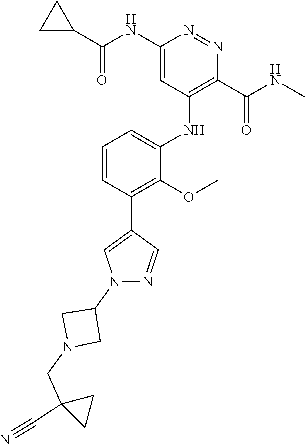 [SMILES: CNC(=O)c1nnc(NC(=O)C2CC2)cc1Nc1cccc(-c2cnn(C3CN(CC4(C#N)CC4)C3)c2)c1OC] | A | | D | E |
| 122 | 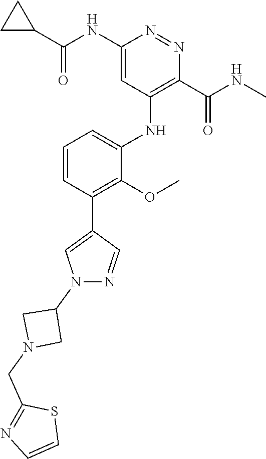 [SMILES: CNC(=O)c1nnc(NC(=O)C2CC2)cc1Nc1cccc(-c2cnn(C3CN(Cc4nccs4)C3)c2)c1OC] | A | | C | C |
| 123 | 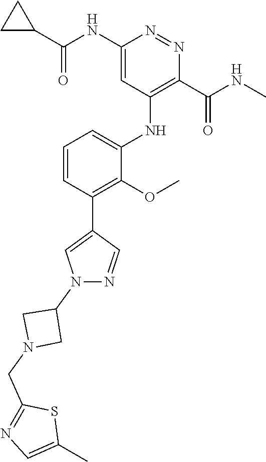 [SMILES: CNC(=O)c1nnc(NC(=O)C2CC2)cc1Nc1cccc(-c2cnn(C3CN(Cc4ncc(C)s4)C3)c2)c1OC] | A | | D | D |
| 124 | 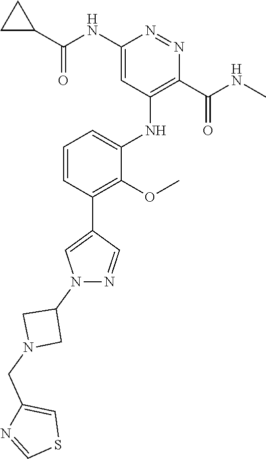 [SMILES: CNC(=O)c1nnc(NC(=O)C2CC2)cc1Nc1cccc(-c2cnn(C3CN(Cc4cscn4)C3)c2)c1OC] | A | | C | C |
| 125 | 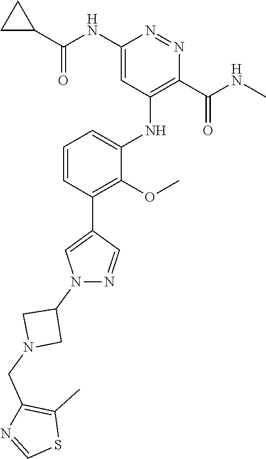 [SMILES: CNC(=O)c1nnc(NC(=O)C2CC2)cc1Nc1cccc(-c2cnn(C3CN(Cc4ncsc4C)C3)c2)c1OC] | A | | D | D |
| 126 | 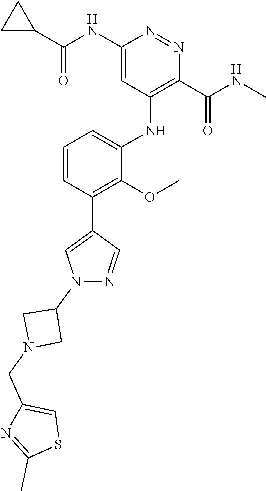 [SMILES: CNC(=O)c1nnc(NC(=O)C2CC2)cc1Nc1cccc(-c2cnn(C3CN(Cc4csc(C)n4)C3)c2)c1OC] | A | | C | C |
| 127 | 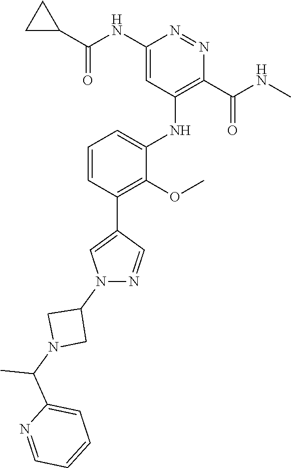 [SMILES: CNC(=O)c1nnc(NC(=O)C2CC2)cc1Nc1cccc(-c2cnn(C3CN(C(C)c4ccccn4)C3)c2)c1OC] | A | | D | D |
| 128 | 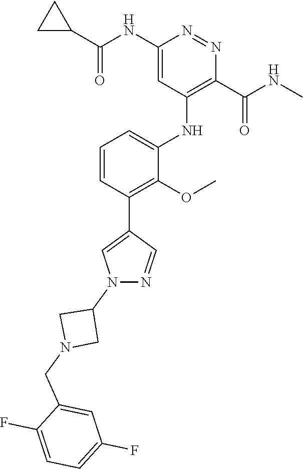 [SMILES: CNC(=O)c1nnc(NC(=O)C2CC2)cc1Nc1cccc(-c2cnn(C3CN(Cc4cc(F)ccc4F)C3)c2)c1OC] | A | | D | D |
| 129 | 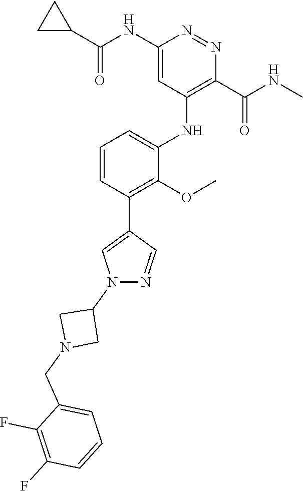 [SMILES: CNC(=O)c1nnc(NC(=O)C2CC2)cc1Nc1cccc(-c2cnn(C3CN(Cc4cccc(F)c4F)C3)c2)c1OC] | B | | D | D |
| 130 | 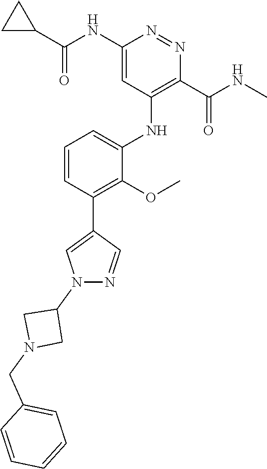 [SMILES: CNC(=O)c1nnc(NC(=O)C2CC2)cc1Nc1cccc(-c2cnn(C3CN(Cc4ccccc4)C3)c2)c1OC] | A | | D | D |
| 131 | 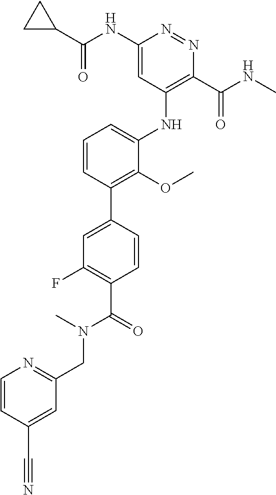 [SMILES: CNC(=O)c1nnc(NC(=O)C2CC2)cc1Nc1cccc(-c2ccc(C(=O)N(C)Cc3cc(C#N)ccn3)c(F)c2)c1OC] | A | | D | D |
| 132 | 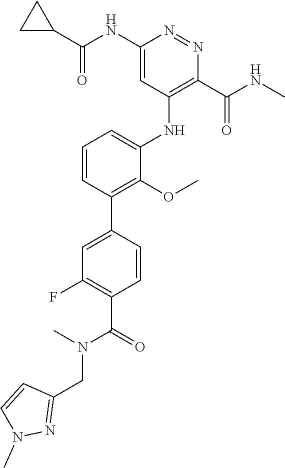 [SMILES: CNC(=O)c1nnc(NC(=O)C2CC2)cc1Nc1cccc(-c2ccc(C(=O)N(C)Cc3ccn(C)n3)c(F)c2)c1OC] | A | | D | D |
| 133 | 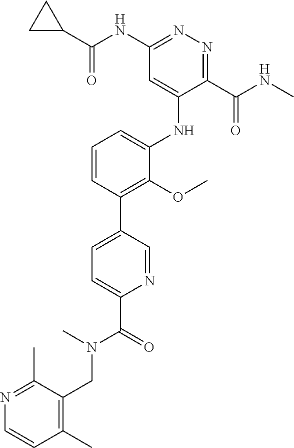 [SMILES: CNC(=O)c1nnc(NC(=O)C2CC2)cc1Nc1cccc(-c2ccc(C(=O)N(C)Cc3c(C)ccnc3C)nc2)c1OC] | A | E | C | C |
| 134 | 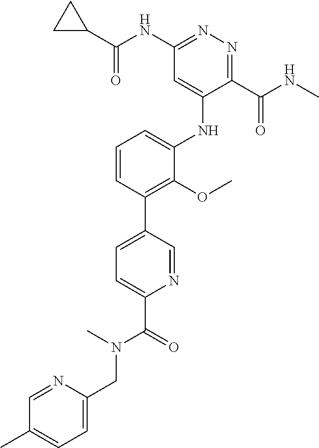 | A | | C | D |
| 135 | 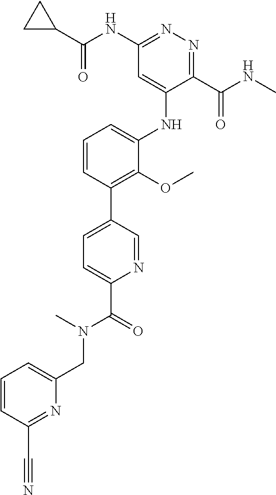 [SMILES: CNC(=O)c1nnc(NC(=O)C2CC2)cc1Nc1cccc(-c2ccc(C(=O)N(C)Cc3cccc(C#N)n3)nc2)c1OC] | A | E | C | C |
| 136 | 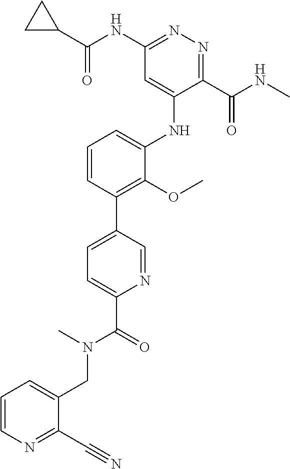 [SMILES: CNC(=O)c1nnc(NC(=O)C2CC2)cc1Nc1cccc(-c2ccc(C(=O)N(C)Cc3cccnc3C#N)nc2)c1OC] | A | E | C | D |
| 137 | 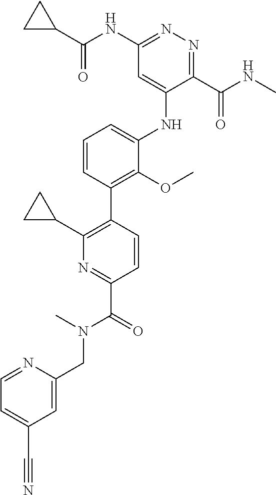 [SMILES: CNC(=O)c1nnc(NC(=O)C2CC2)cc1Nc1cccc(-c2ccc(C(=O)N(C)Cc3cc(C#N)ccn3)nc2C2CC2)c1OC] | B | | E | E |
| 138 | 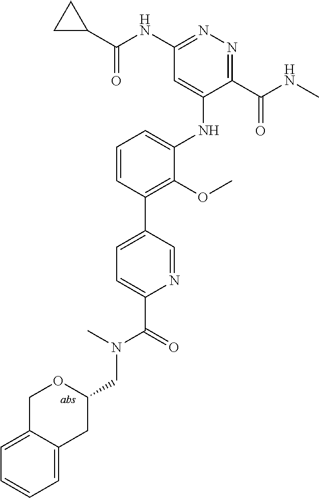 [SMILES: CNC(=O)c1nnc(NC(=O)C2CC2)cc1Nc1cccc(-c2ccc(C(=O)N(C)C[C@@H]3Cc4ccccc4CO3)nc2)c1OC] | B | | D | E |
| 139 | 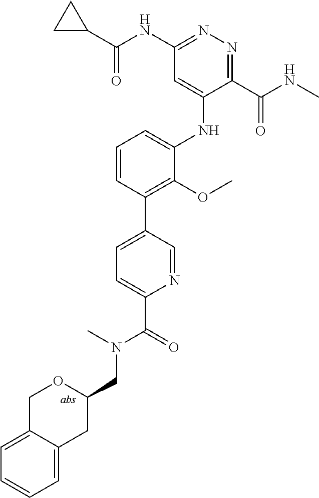 [SMILES: CNC(=O)c1nnc(NC(=O)C2CC2)cc1Nc1cccc(-c2ccc(C(=O)N(C)C[C@H]3Cc4ccccc4CO3)nc2)c1OC] | B | | D | E |
| 140 | 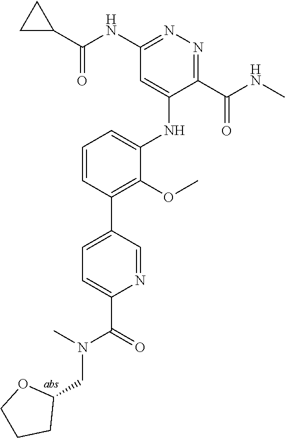 [SMILES: CNC(=O)c1nnc(NC(=O)C2CC2)cc1Nc1cccc(-c2ccc(C(=O)N(C)C[C@@H]3CCCO3)nc2)c1OC] | B | | E | E |
| 141 | 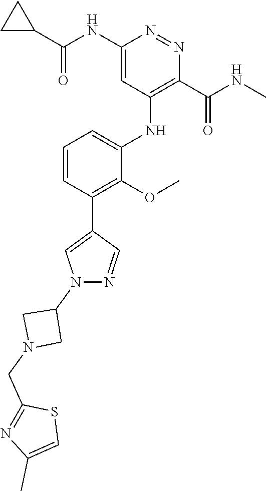 [SMILES: CNC(=O)c1nnc(NC(=O)C2CC2)cc1Nc1cccc(-c2cnn(C3CN(Cc4nc(C)cs4)C3)c2)c1OC] | A | E | D | D |
| 142 | 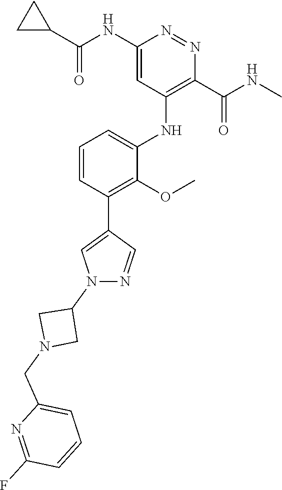 [SMILES: CNC(=O)c1nnc(NC(=O)C2CC2)cc1Nc1cccc(-c2cnn(C3CN(Cc4cccc(F)n4)C3)c2)c1OC] | A | D | C | C |
| 143 | 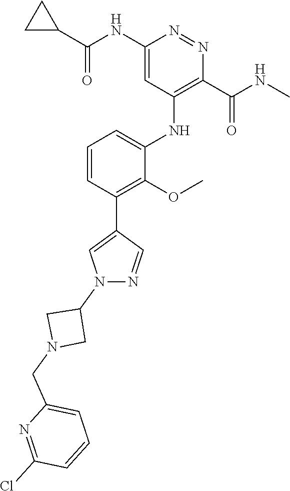 [SMILES: CNC(=O)c1nnc(NC(=O)C2CC2)cc1Nc1cccc(-c2cnn(C3CN(Cc4cccc(Cl)n4)C3)c2)c1OC] | A | E | C | C |
| 144 | 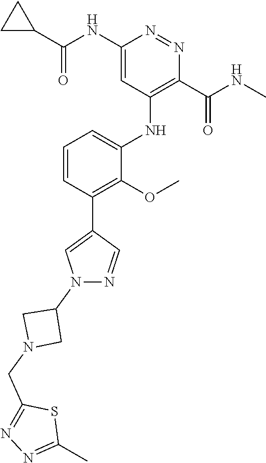 [SMILES: CNC(=O)c1nnc(NC(=O)C2CC2)cc1Nc1cccc(-c2cnn(C3CN(Cc4nnc(C)s4)C3)c2)c1OC] | A | | D | D |
| 145 | 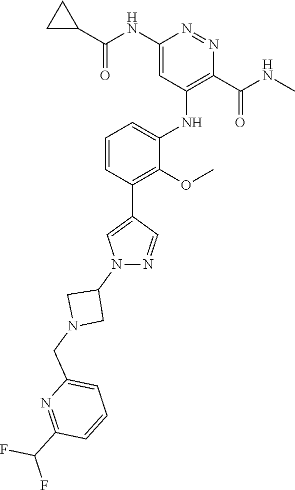 [SMILES: CNC(=O)c1nnc(NC(=O)C2CC2)cc1Nc1cccc(-c2cnn(C3CN(Cc4cccc(C(F)F)n4)C3)c2)c1OC] | A | E | C | C |
| 146 | 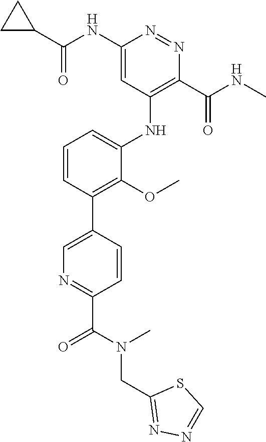 [SMILES: CNC(=O)c1nnc(NC(=O)C2CC2)cc1Nc1cccc(-c2ccc(C(=O)N(C)Cc3nncs3)nc2)c1OC] | A | E | D | E |
| 147 | 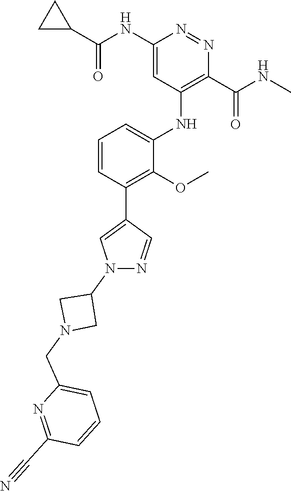 [SMILES: CNC(=O)c1nnc(NC(=O)C2CC2)cc1Nc1cccc(-c2cnn(C3CN(Cc4cccc(C#N)n4)C3)c2)c1OC] | A | D | C | C |
| 148 | 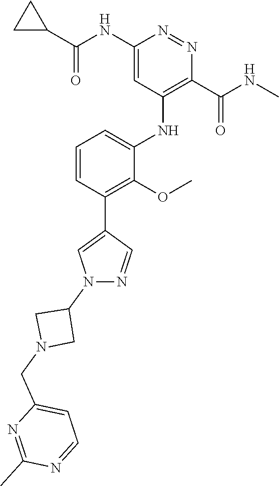 | A | D | D | E |
| 149 | 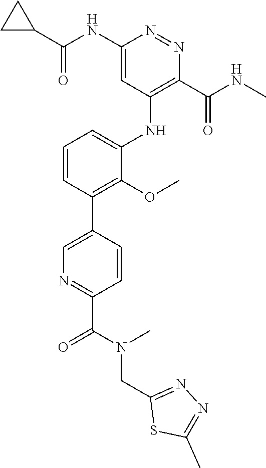 [SMILES: CNC(=O)c1nnc(NC(=O)C2CC2)cc1Nc1cccc(-c2ccc(C(=O)N(C)Cc3nnc(C)s3)nc2)c1OC] | A | E | D | D |
| 150 | 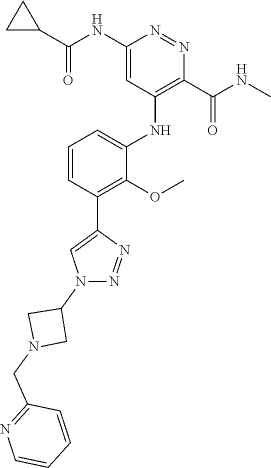 [SMILES: CNC(=O)c1nnc(NC(=O)C2CC2)cc1Nc1cccc(-c2cn(C3CN(Cc4ccccn4)C3)nn2)c1OC] | A | E | D | D |
| 151 | 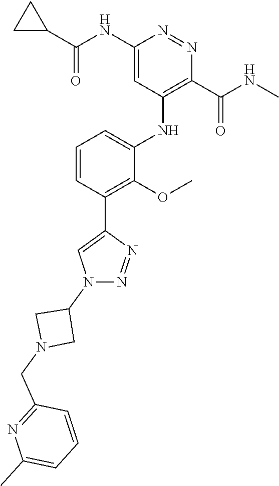 | A | D | C | C |
| 152 | 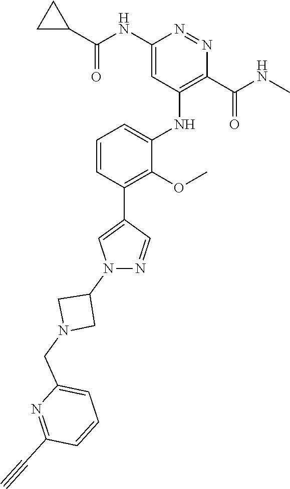 [SMILES: C#Cc1cccc(CN2CC(n3cc(-c4cccc(Nc5cc(NC(=O)C6CC6)nnc5C(=O)NC)c4OC)cn3)C2)n1] | A | D | C | C |
| 153 | 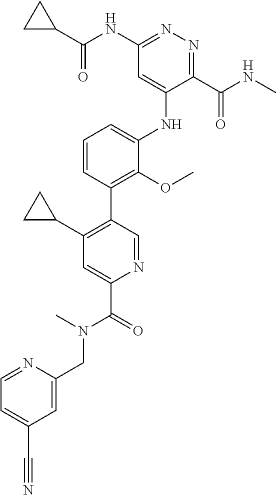 [SMILES: CNC(=O)c1nnc(NC(=O)C2CC2)cc1Nc1cccc(-c2cnc(C(=O)N(C)Cc3cc(C#N)ccn3)cc2C2CC2)c1OC] | A | | C | C |
| 154 | 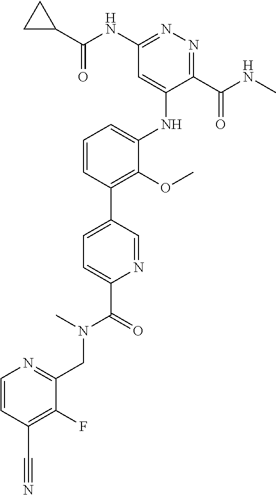 [SMILES: CNC(=O)c1nnc(NC(=O)C2CC2)cc1Nc1cccc(-c2ccc(C(=O)N(C)Cc3nccc(C#N)c3F)nc2)c1OC] | A | | E | E |
| 155 | 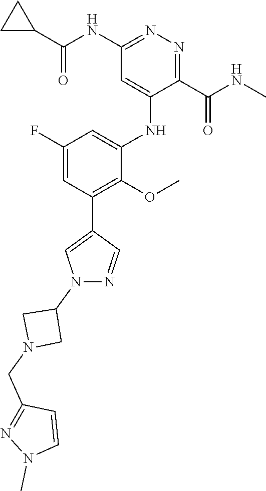 [SMILES: CNC(=O)c1nnc(NC(=O)C2CC2)cc1Nc1cc(F)cc(-c2cnn(C3CN(Cc4ccn(C)n4)C3)c2)c1OC] | A | E | D | D |
| 156 | 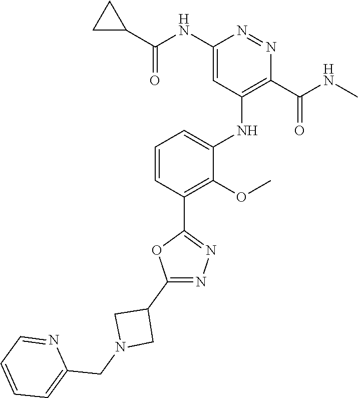 [SMILES: CNC(=O)c1nnc(NC(=O)C2CC2)cc1Nc1cccc(-c2nnc(C3CN(Cc4ccccn4)C3)o2)c1OC] | B | E | E | E |
| 157 | 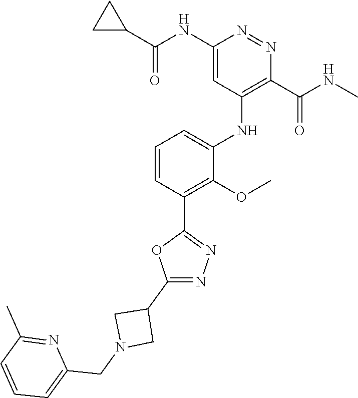 [SMILES: CNC(=O)c1nnc(NC(=O)C2CC2)cc1Nc1cccc(-c2nnc(C3CN(Cc4cccc(C)n4)C3)o2)c1OC] | A | E | E | E |
| 158 | 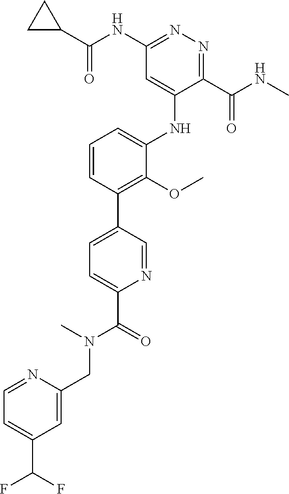 [SMILES: CNC(=O)c1nnc(NC(=O)C2CC2)cc1Nc1cccc(-c2ccc(C(=O)N(C)Cc3cc(C(F)F)ccn3)nc2)c1OC] | A | E | C | C |
| 159 | 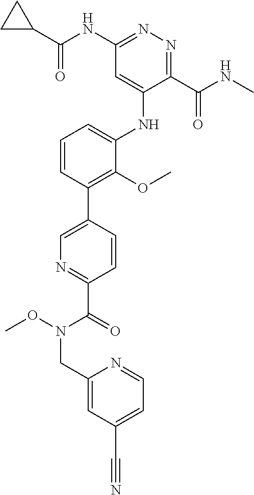 [SMILES: CNC(=O)c1nnc(NC(=O)C2CC2)cc1Nc1cccc(-c2ccc(C(=O)N(Cc3cc(C#N)ccn3)OC)nc2)c1OC] | A | E | C | C |
| 160 | 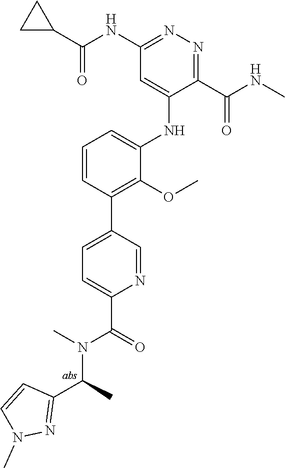 [SMILES: CNC(=O)c1nnc(NC(=O)C2CC2)cc1Nc1cccc(-c2ccc(C(=O)N(C)[C@@H](C)c3ccn(C)n3)nc2)c1OC] | A | | D | D |
| 161 | 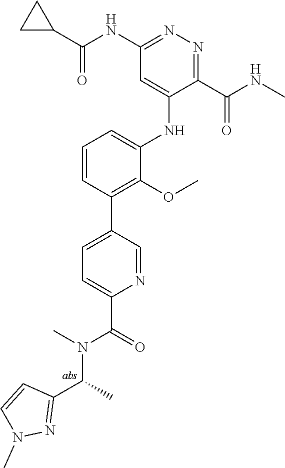 [SMILES: CNC(=O)c1nnc(NC(=O)C2CC2)cc1Nc1cccc(-c2ccc(C(=O)N(C)[C@H](C)c3ccn(C)n3)nc2)c1OC] | A | | D | D |
| 162 | 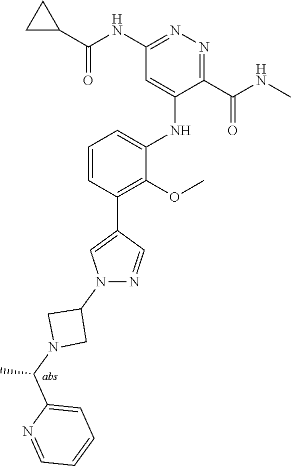 [SMILES: CNC(=O)c1nnc(NC(=O)C2CC2)cc1Nc1cccc(-c2cnn(C3CN([C@@H](C)c4ccccn4)C3)c2)c1OC] | A | | C | D |
| 163 | 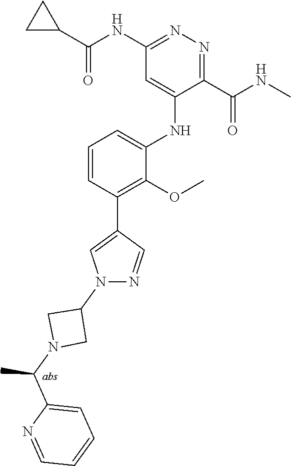 [SMILES: CNC(=O)c1nnc(NC(=O)C2CC2)cc1Nc1cccc(-c2cnn(C3CN([C@H](C)c4ccccn4)C3)c2)c1OC] | A | | D | E |
| 164 | 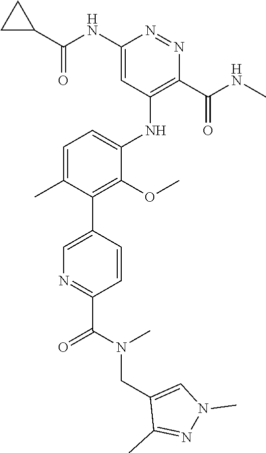 [SMILES: CNC(=O)c1nnc(NC(=O)C2CC2)cc1Nc1ccc(C)c(-c2ccc(C(=O)N(C)Cc3cn(C)nc3C)nc2)c1OC] | A | | C | C |
| 165 | 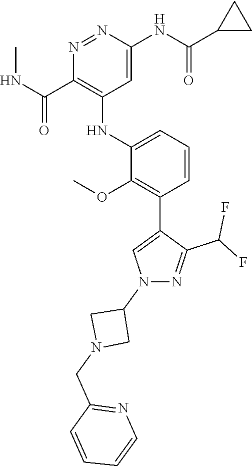 [SMILES: CNC(=O)c1nnc(NC(=O)C2CC2)cc1Nc1cccc(-c2cn(C3CN(Cc4ccccn4)C3)nc2C(F)F)c1OC] | A | | D | D |
| 166 | 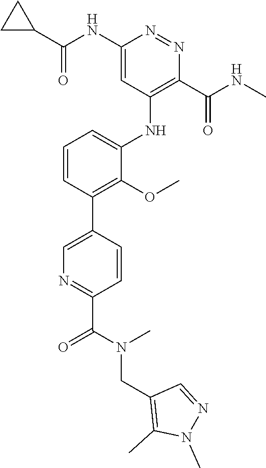 [SMILES: CNC(=O)c1nnc(NC(=O)C2CC2)cc1Nc1cccc(-c2ccc(C(=O)N(C)Cc3cnn(C)c3C)nc2)c1OC] | A | | D | D |
| 167 | 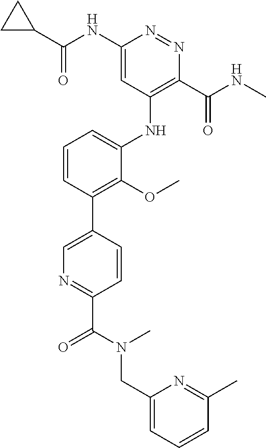 [SMILES: CNC(=O)c1nnc(NC(=O)C2CC2)cc1Nc1cccc(-c2ccc(C(=O)N(C)Cc3cccc(C)n3)nc2)c1OC] | A | | C | C |
| 168 | 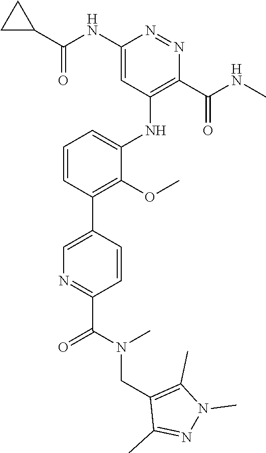 [SMILES: CNC(=O)c1nnc(NC(=O)C2CC2)cc1Nc1cccc(-c2ccc(C(=O)N(C)Cc3c(C)nn(C)c3C)nc2)c1OC] | A | | C | C |
| 169 | 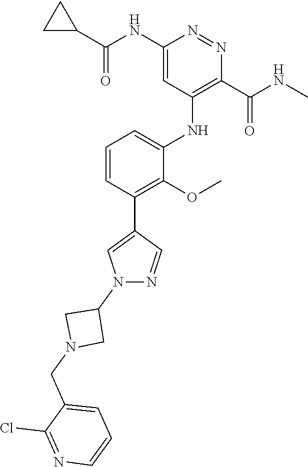 [SMILES: CNC(=O)c1nnc(NC(=O)C2CC2)cc1Nc1cccc(-c2cnn(C3CN(Cc4cccnc4Cl)C3)c2)c1OC] | A | | C | C |
| 170 | 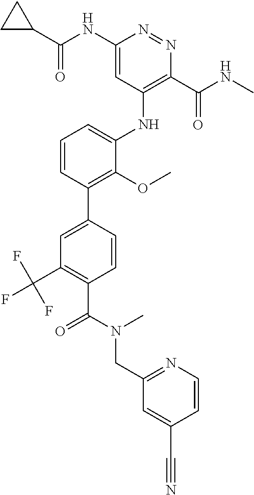 [SMILES: CNC(=O)c1nnc(NC(=O)C2CC2)cc1Nc1cccc(-c2ccc(C(=O)N(C)Cc3cc(C#N)ccn3)c(C(F)(F)F)c2)c1OC] | A | | D | D |
| 171 | 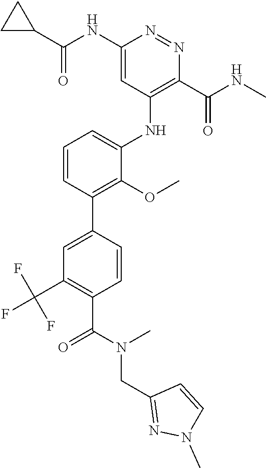 [SMILES: CNC(=O)c1nnc(NC(=O)C2CC2)cc1Nc1cccc(-c2ccc(C(=O)N(C)Cc3ccn(C)n3)c(C(F)(F)F)c2)c1OC] | A | | D | D |
| 172 | 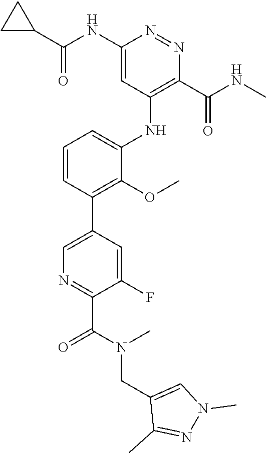 [SMILES: CNC(=O)c1nnc(NC(=O)C2CC2)cc1Nc1cccc(-c2cnc(C(=O)N(C)Cc3cn(C)nc3C)c(F)c2)c1OC] | A | | C | C |
| 173 | 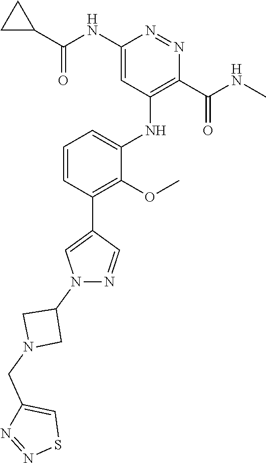 [SMILES: CNC(=O)c1nnc(NC(=O)C2CC2)cc1Nc1cccc(-c2cnn(C3CN(Cc4csnn4)C3)c2)c1OC] | A | | C | C |
| 174 | 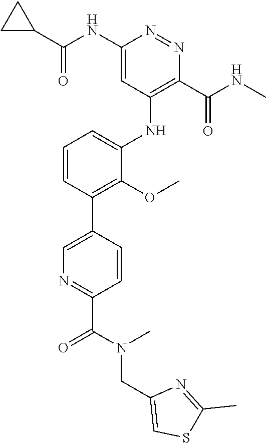 [SMILES: CNC(=O)c1nnc(NC(=O)C2CC2)cc1Nc1cccc(-c2ccc(C(=O)N(C)Cc3csc(C)n3)nc2)c1OC] | A | | C | C |
| 175 | 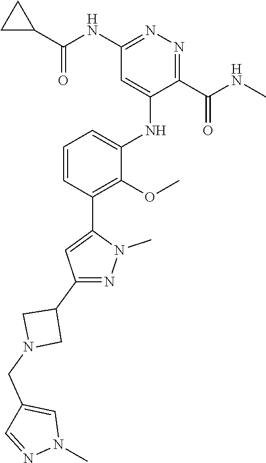 [SMILES: CNC(=O)c1nnc(NC(=O)C2CC2)cc1Nc1cccc(-c2cc(C3CN(Cc4cnn(C)c4)C3)nn2C)c1OC] | A | | E | E |
| 176 | 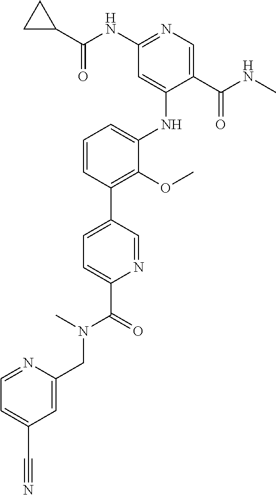 [SMILES: CNC(=O)c1cnc(NC(=O)C2CC2)cc1Nc1cccc(-c2ccc(C(=O)N(C)Cc3cc(C#N)ccn3)nc2)c1OC] | A | | D | C |
| 177 | 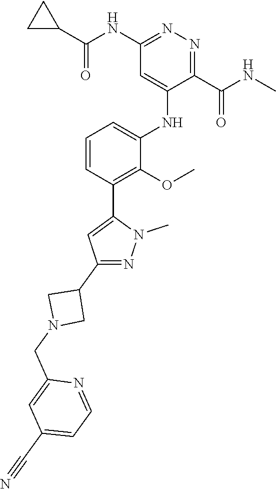 [SMILES: CNC(=O)c1nnc(NC(=O)C2CC2)cc1Nc1cccc(-c2cc(C3CN(Cc4cc(C#N)ccn4)C3)nn2C)c1OC] | A | | E | E |
| 178 | 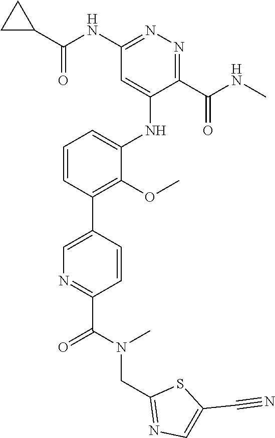 [SMILES: CNC(=O)c1nnc(NC(=O)C2CC2)cc1Nc1cccc(-c2ccc(C(=O)N(C)Cc3ncc(C#N)s3)nc2)c1OC] | A | | E | E |
| 179 | 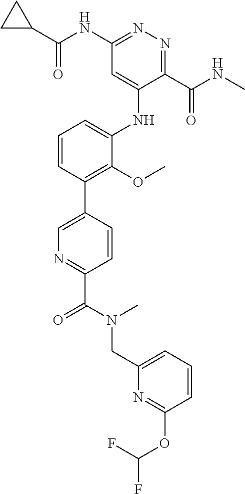 [SMILES: CNC(=O)c1nnc(NC(=O)C2CC2)cc1Nc1cccc(-c2ccc(C(=O)N(C)Cc3cccc(OC(F)F)n3)nc2)c1OC] | A | | D | D |
| 180 | 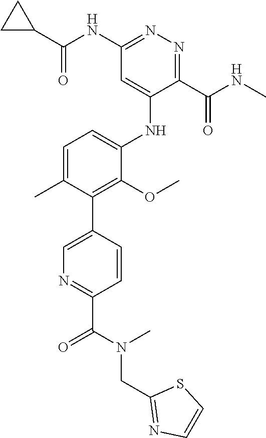 [SMILES: CNC(=O)c1nnc(NC(=O)C2CC2)cc1Nc1ccc(C)c(-c2ccc(C(=O)N(C)Cc3nccs3)nc2)c1OC] | A | | D | D |
| 181 | 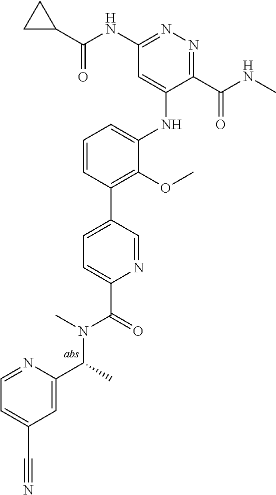 [SMILES: CNC(=O)c1nnc(NC(=O)C2CC2)cc1Nc1cccc(-c2ccc(C(=O)N(C)[C@H](C)c3cc(C#N)ccn3)nc2)c1OC] | A | | D | D |
| 182 | 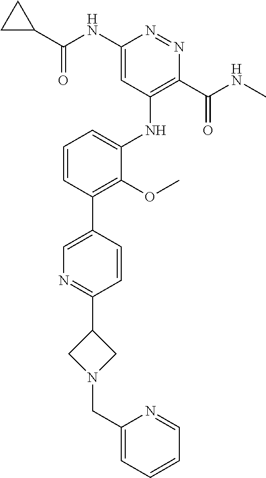 [SMILES: CNC(=O)c1nnc(NC(=O)C2CC2)cc1Nc1cccc(-c2ccc(C3CN(Cc4ccccn4)C3)nc2)c1OC] | A | | E | D |
| 183 | 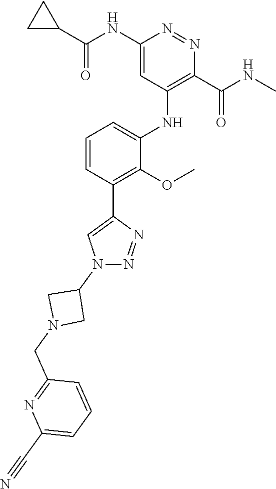 [SMILES: CNC(=O)c1nnc(NC(=O)C2CC2)cc1Nc1cccc(-c2cn(C3CN(Cc4cccc(C#N)n4)C3)nn2)c1OC] | A | | D | C |
| 184 | 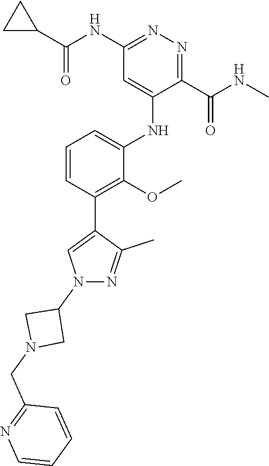 [SMILES: CNC(=O)c1nnc(NC(=O)C2CC2)cc1Nc1cccc(-c2cn(C3CN(Cc4ccccn4)C3)nc2C)c1OC] | A | | D | D |
| 185 | 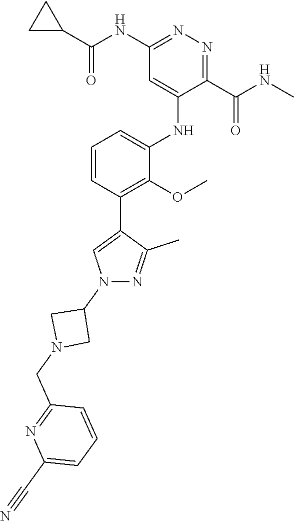 [SMILES: CNC(=O)c1nnc(NC(=O)C2CC2)cc1Nc1cccc(-c2cn(C3CN(Cc4cccc(C#N)n4)C3)nc2C)c1OC] | A | | D | D |
| 186 | 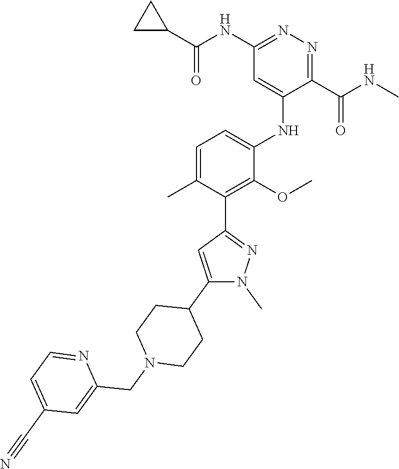 [SMILES: CNC(=O)c1nnc(NC(=O)C2CC2)cc1Nc1ccc(C)c(-c2cc(C3CCN(Cc4cc(C#N)ccn4)CC3)n(C)n2)c1OC] | C | | E | E |
| 187 | 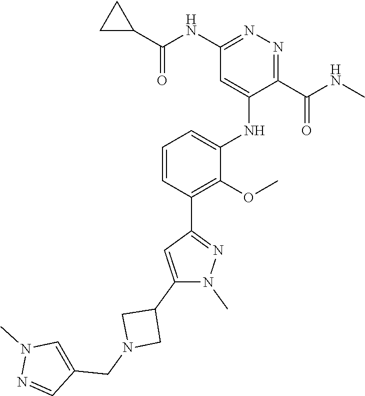 | C | | E | E |
| 188 | 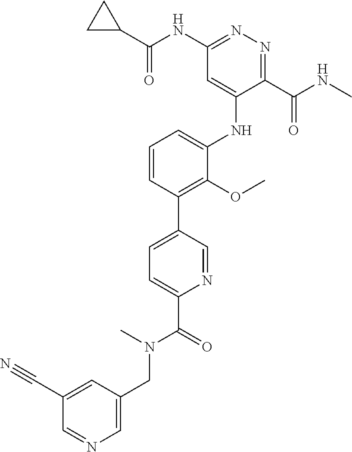 [SMILES: CNC(=O)c1nnc(NC(=O)C2CC2)cc1Nc1cccc(-c2ccc(C(=O)N(C)Cc3cncc(C#N)c3)nc2)c1OC] | A | | D | D |
| 189 | 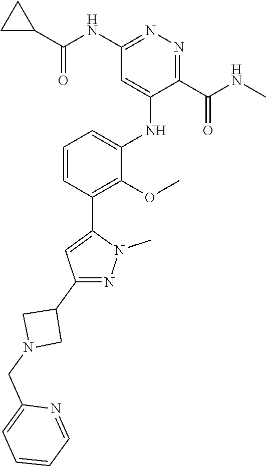 [SMILES: CNC(=O)c1nnc(NC(=O)C2CC2)cc1Nc1cccc(-c2cc(C3CN(Cc4ccccn4)C3)nn2C)c1OC] | B | | E | E |
| 190 | 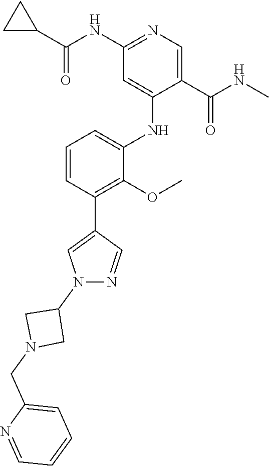 [SMILES: CNC(=O)c1cnc(NC(=O)C2CC2)cc1Nc1cccc(-c2cnn(C3CN(Cc4ccccn4)C3)c2)c1OC] | A | | D | D |
| 191 | 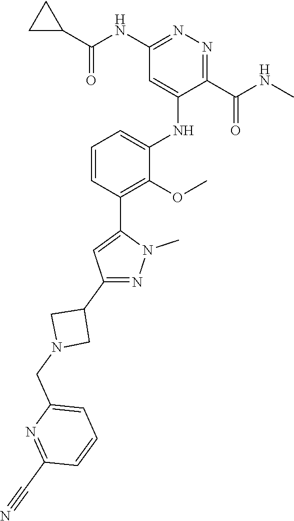 [SMILES: CNC(=O)c1nnc(NC(=O)C2CC2)cc1Nc1cccc(-c2cc(C3CN(Cc4cccc(C#N)n4)C3)nn2C)c1OC] | B | | E | E |
| 192 | 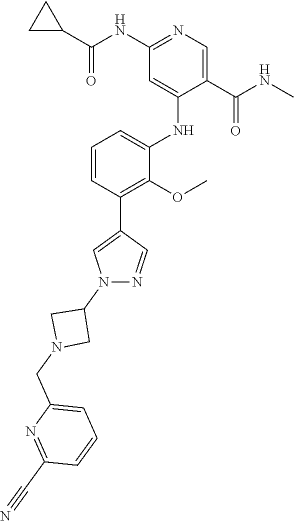 [SMILES: CNC(=O)c1cnc(NC(=O)C2CC2)cc1Nc1cccc(-c2cnn(C3CN(Cc4cccc(C#N)n4)C3)c2)c1OC] | A | | C | C |
| 193 | 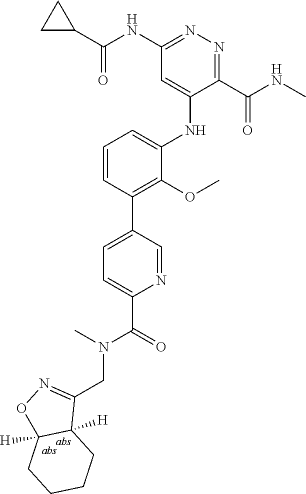 [SMILES: CNC(=O)c1nnc(NC(=O)C2CC2)cc1Nc1cccc(-c2ccc(C(=O)N(C)CC3=NO[C@@H]4CCCC[C@H]34)nc2)c1OC] | A | | C | C |
| 194 | 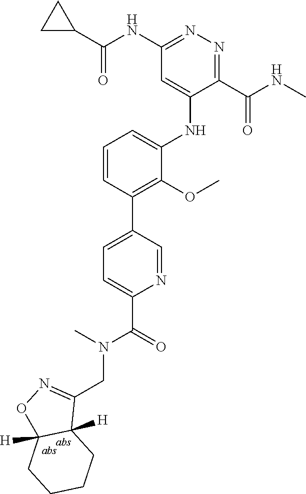 [SMILES: CNC(=O)c1nnc(NC(=O)C2CC2)cc1Nc1cccc(-c2ccc(C(=O)N(C)CC3=NO[C@H]4CCCC[C@@H]34)nc2)c1OC] | A | | D | D |
| 195 | 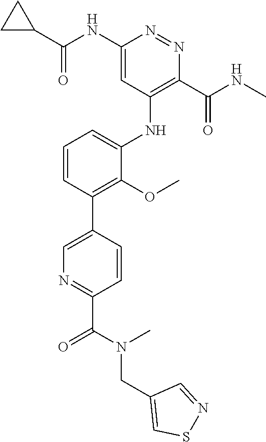 [SMILES: CNC(=O)c1nnc(NC(=O)C2CC2)cc1Nc1cccc(-c2ccc(C(=O)N(C)Cc3cnsc3)nc2)c1OC] | A | | D | D |
| 196 | 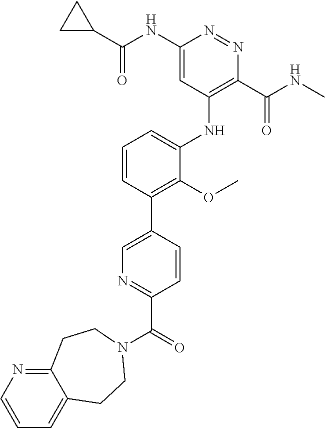 [SMILES: CNC(=O)c1nnc(NC(=O)C2CC2)cc1Nc1cccc(-c2ccc(C(=O)N3CCc4cccnc4CC3)nc2)c1OC] | A | | D | D |
| 197 | 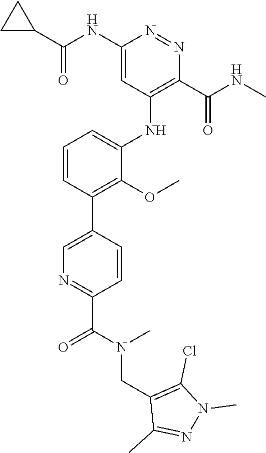 [SMILES: CNC(=O)c1nnc(NC(=O)C2CC2)cc1Nc1cccc(-c2ccc(C(=O)N(C)Cc3c(C)nn(C)c3Cl)nc2)c1OC] | A | | C | C |
| 198 | 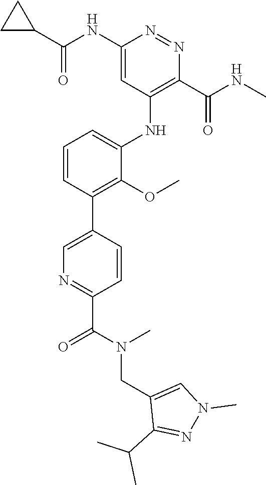 [SMILES: CNC(=O)c1nnc(NC(=O)C2CC2)cc1Nc1cccc(-c2ccc(C(=O)N(C)Cc3cn(C)nc3C(C)C)nc2)c1OC] | A | | C | C |
| 199 | 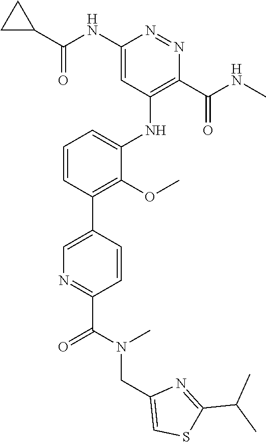 [SMILES: CNC(=O)c1nnc(NC(=O)C2CC2)cc1Nc1cccc(-c2ccc(C(=O)N(C)Cc3csc(C(C)C)n3)nc2)c1OC] | A | | C | C |
| 200 | 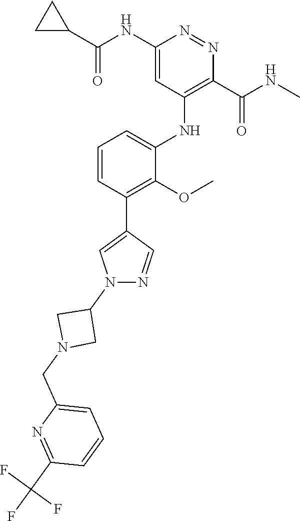 [SMILES: CNC(=O)c1nnc(NC(=O)C2CC2)cc1Nc1cccc(-c2cnn(C3CN(Cc4cccc(C(F)(F)F)n4)C3)c2)c1OC] | A | | C | C |
| 201 | 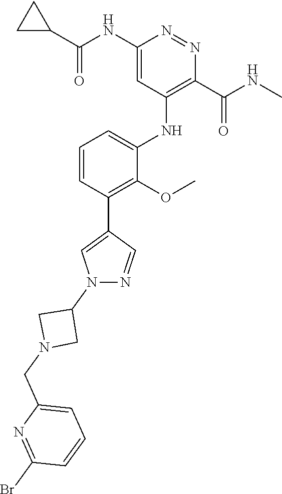 [SMILES: CNC(=O)c1nnc(NC(=O)C2CC2)cc1Nc1cccc(-c2cnn(C3CN(Cc4cccc(Br)n4)C3)c2)c1OC] | A | | C | C |
| 202 | 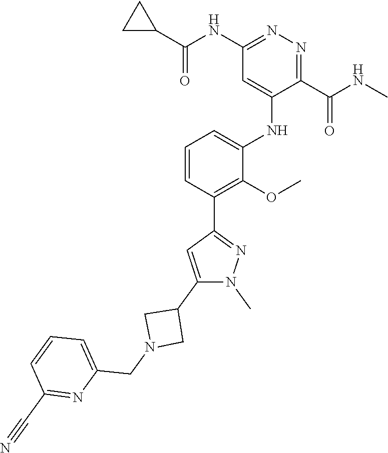 [SMILES: CNC(=O)c1nnc(NC(=O)C2CC2)cc1Nc1cccc(-c2cc(C3CN(Cc4cccc(C#N)n4)C3)n(C)n2)c1OC] | C | | E | E |
| 203 | 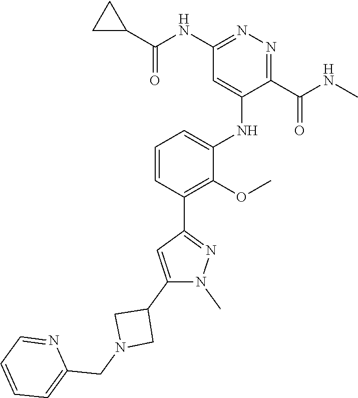 [SMILES: CNC(=O)c1nnc(NC(=O)C2CC2)cc1Nc1cccc(-c2cc(C3CN(Cc4ccccn4)C3)n(C)n2)c1OC] | C | | E | E |
| 204 | 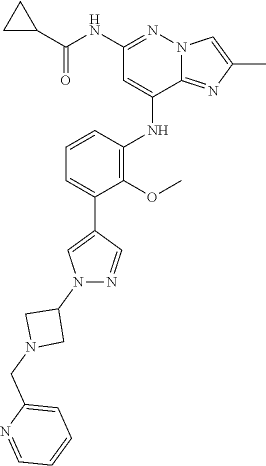 [SMILES: COc1c(Nc2cc(NC(=O)C3CC3)nn3cc(C)nc23)cccc1-c1cnn(C2CN(Cc3ccccn3)C2)c1] | A | | D | D |
| 205 | 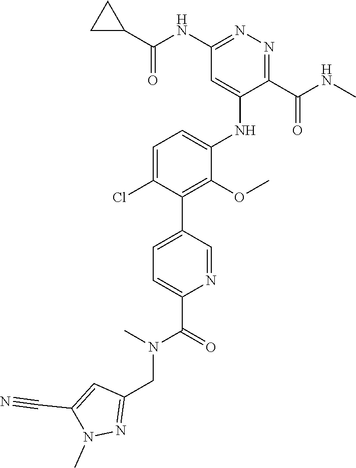 [SMILES: CNC(=O)c1nnc(NC(=O)C2CC2)cc1Nc1ccc(Cl)c(-c2ccc(C(=O)N(C)Cc3cc(C#N)n(C)n3)nc2)c1OC] | A | | C | C |
| 206 | 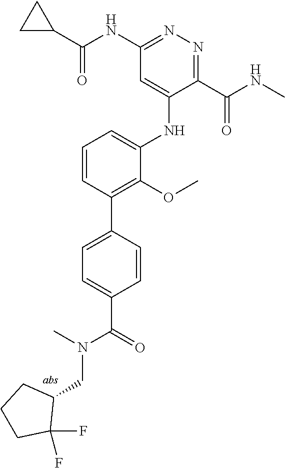 [SMILES: CNC(=O)c1nnc(NC(=O)C2CC2)cc1Nc1cccc(-c2ccc(C(=O)N(C)C[C@H]3CCCC3(F)F)cc2)c1OC] | B | | E | E |
| 207 | 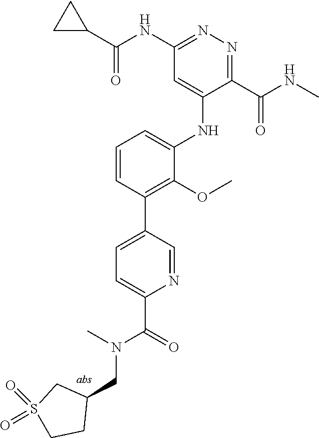 [SMILES: CNC(=O)c1nnc(NC(=O)C2CC2)cc1Nc1cccc(-c2ccc(C(=O)N(C)C[C@H]3CCS(=O)(=O)C3)nc2)c1OC] | A | | E | E |
| 208 | 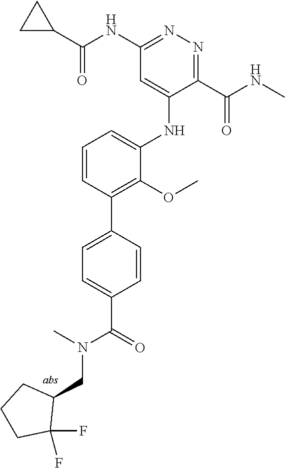 [SMILES: CNC(=O)c1nnc(NC(=O)C2CC2)cc1Nc1cccc(-c2ccc(C(=O)N(C)C[C@@H]3CCCC3(F)F)cc2)c1OC] | A | | E | E |
| 209 | 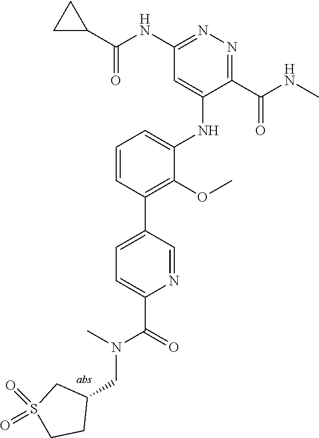 [SMILES: CNC(=O)c1nnc(NC(=O)C2CC2)cc1Nc1cccc(-c2ccc(C(=O)N(C)C[C@@H]3CCS(=O)(=O)C3)nc2)c1OC] | B | | E | E |
| 210 | 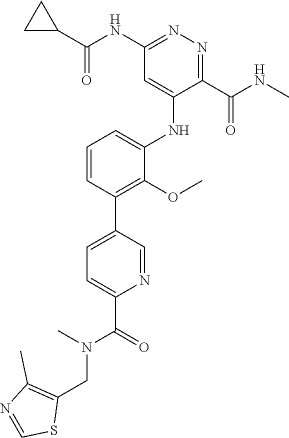 [SMILES: CNC(=O)c1nnc(NC(=O)C2CC2)cc1Nc1cccc(-c2ccc(C(=O)N(C)Cc3scnc3C)nc2)c1OC] | A | | E | D |
| 211 | 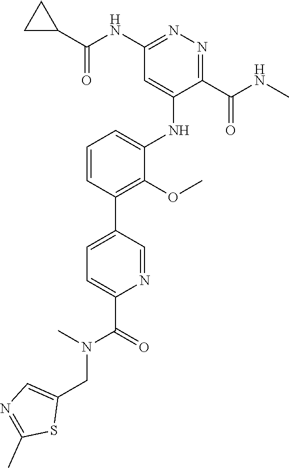 [SMILES: CNC(=O)c1nnc(NC(=O)C2CC2)cc1Nc1cccc(-c2ccc(C(=O)N(C)Cc3cnc(C)s3)nc2)c1OC] | A | | E | D |
| 212 | 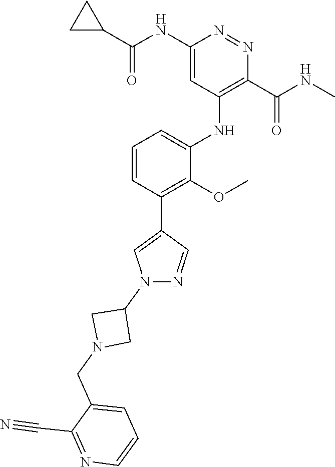 [SMILES: CNC(=O)c1nnc(NC(=O)C2CC2)cc1Nc1cccc(-c2cnn(C3CN(Cc4cccnc4C#N)C3)c2)c1OC] | A | | C | C |
| 213 | 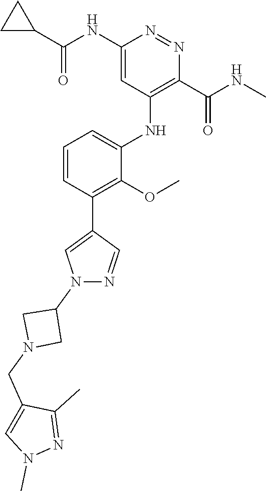 [SMILES: CNC(=O)c1nnc(NC(=O)C2CC2)cc1Nc1cccc(-c2cnn(C3CN(Cc4cn(C)nc4C)C3)c2)c1OC] | A | | D | D |
| 214 | 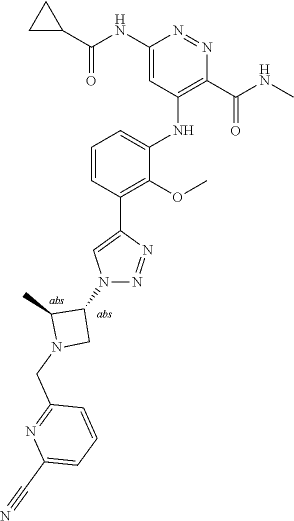 [SMILES: CNC(=O)c1nnc(NC(=O)C2CC2)cc1Nc1cccc(-c2cn([C@@H]3CN(Cc4cccc(C#N)n4)[C@H]3C)nn2)c1OC] | A | | C | C |
| 215 | 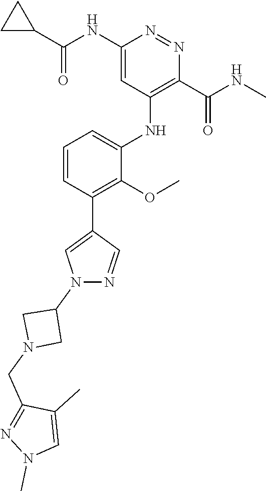 [SMILES: CNC(=O)c1nnc(NC(=O)C2CC2)cc1Nc1cccc(-c2cnn(C3CN(Cc4nn(C)cc4C)C3)c2)c1OC] | 0.002 | | D | D |
| 216 | 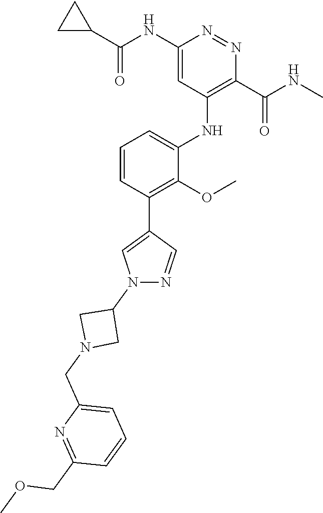 | A | | C | C |
| 217 | 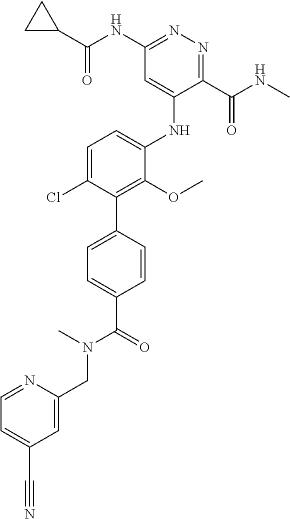 [SMILES: CNC(=O)c1nnc(NC(=O)C2CC2)cc1Nc1ccc(Cl)c(-c2ccc(C(=O)N(C)Cc3cc(C#N)ccn3)cc2)c1OC] | A | | D | D |
| 218 | 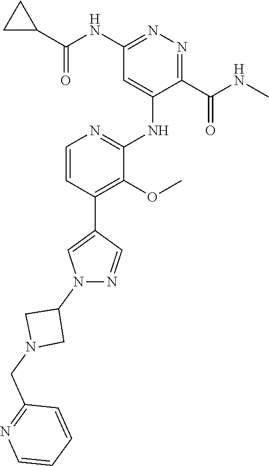 [SMILES: CNC(=O)c1nnc(NC(=O)C2CC2)cc1Nc1nccc(-c2cnn(C3CN(Cc4ccccn4)C3)c2)c1OC] | A | | C | D |
| 219 | 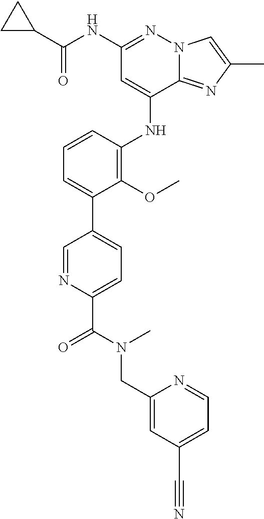 [SMILES: COc1c(Nc2cc(NC(=O)C3CC3)nn3cc(C)nc23)cccc1-c1ccc(C(=O)N(C)Cc2cc(C#N)ccn2)nc1] | A | | D | D |
| 220 | 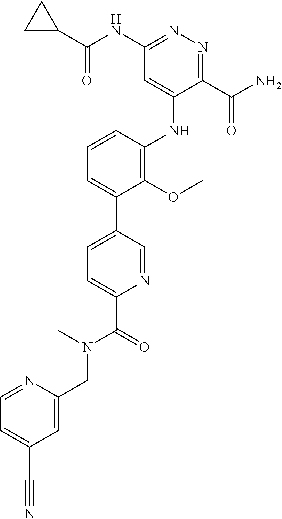 [SMILES: COc1c(Nc2cc(NC(=O)C3CC3)nnc2C(N)=O)cccc1-c1ccc(C(=O)N(C)Cc2cc(C#N)ccn2)nc1] | A | C | C | C |
| 221 | 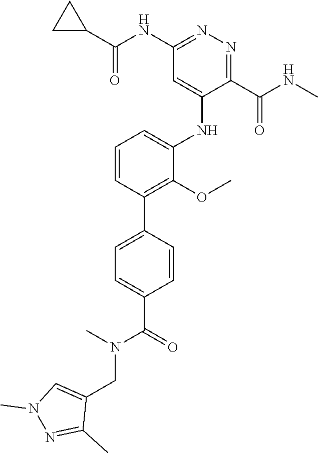 | A | | C | C |
| 222 | 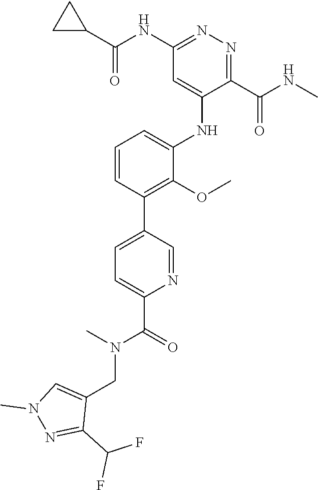 | A | | C | C |
| 223 | 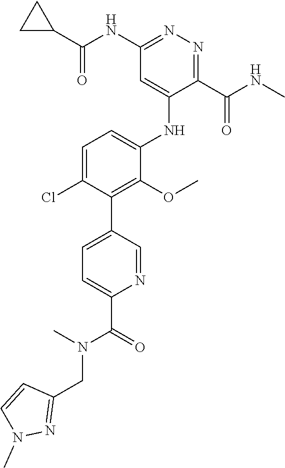 [SMILES: CNC(=O)c1nnc(NC(=O)C2CC2)cc1Nc1ccc(Cl)c(-c2ccc(C(=O)N(C)Cc3ccn(C)n3)nc2)c1OC] | A | | D | C |
| 224 | 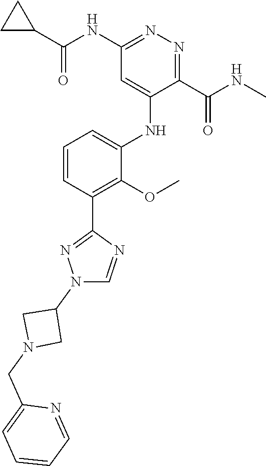 [SMILES: CNC(=O)c1nnc(NC(=O)C2CC2)cc1Nc1cccc(-c2ncn(C3CN(Cc4ccccn4)C3)n2)c1OC] | A | | E | E |
| 225 | 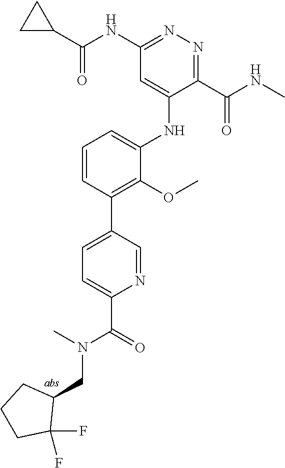 [SMILES: CNC(=O)c1nnc(NC(=O)C2CC2)cc1Nc1cccc(-c2ccc(C(=O)N(C)C[C@@H]3CCCC3(F)F)nc2)c1OC] | B | | E | E |
| 226 | 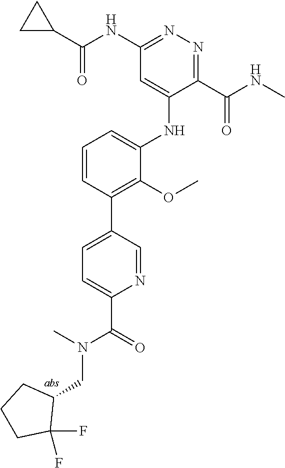 [SMILES: CNC(=O)c1nnc(NC(=O)C2CC2)cc1Nc1cccc(-c2ccc(C(=O)N(C)C[C@H]3CCCC3(F)F)nc2)c1OC] | B | | E | E |
| 227 | 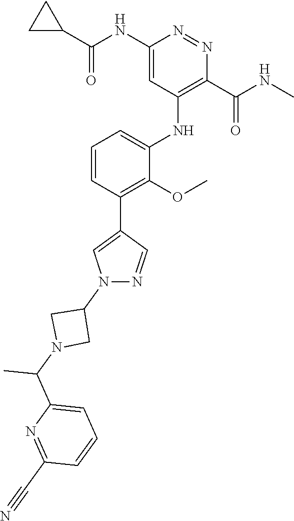 [SMILES: CNC(=O)c1nnc(NC(=O)C2CC2)cc1Nc1cccc(-c2cnn(C3CN(C(C)c4cccc(C#N)n4)C3)c2)c1OC] | A | | D | D |
| 228 | 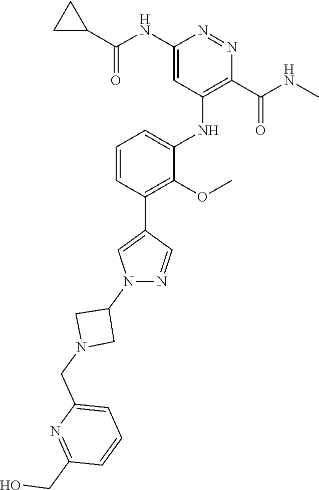 [SMILES: CNC(=O)c1nnc(NC(=O)C2CC2)cc1Nc1cccc(-c2cnn(C3CN(Cc4cccc(CO)n4)C3)c2)c1OC] | A | | C | C |
| 229 | 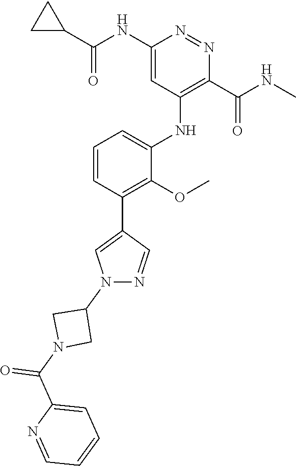 [SMILES: CNC(=O)c1nnc(NC(=O)C2CC2)cc1Nc1cccc(-c2cnn(C3CN(C(=O)c4ccccn4)C3)c2)c1OC] | A | | D | D |
| 230 | 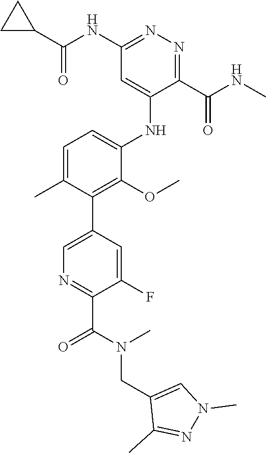 [SMILES: CNC(=O)c1nnc(NC(=O)C2CC2)cc1Nc1ccc(C)c(-c2cnc(C(=O)N(C)Cc3cn(C)nc3C)c(F)c2)c1OC] | A | | C | C |
| 231 | 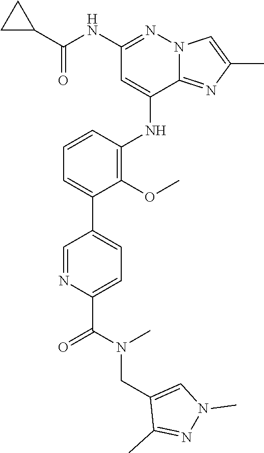 [SMILES: COc1c(Nc2cc(NC(=O)C3CC3)nn3cc(C)nc23)cccc1-c1ccc(C(=O)N(C)Cc2cn(C)nc2C)nc1] | A | | D | D |
| 232 | 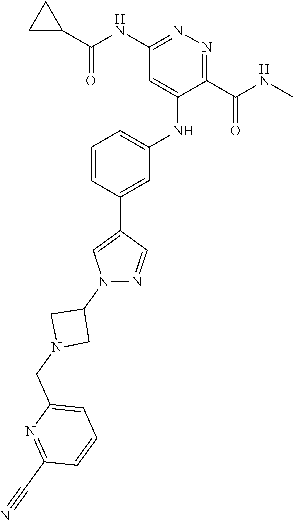 [SMILES: CNC(=O)c1nnc(NC(=O)C2CC2)cc1Nc1cccc(-c2cnn(C3CN(Cc4cccc(C#N)n4)C3)c2)c1] | B | | E | E |
| 233 | 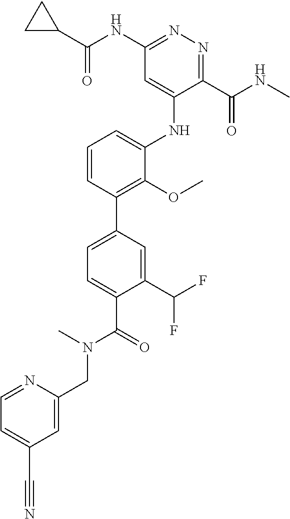 [SMILES: CNC(=O)c1nnc(NC(=O)C2CC2)cc1Nc1cccc(-c2ccc(C(=O)N(C)Cc3cc(C#N)ccn3)c(C(F)F)c2)c1OC] | 0.004 | | D | D |
| 234 | 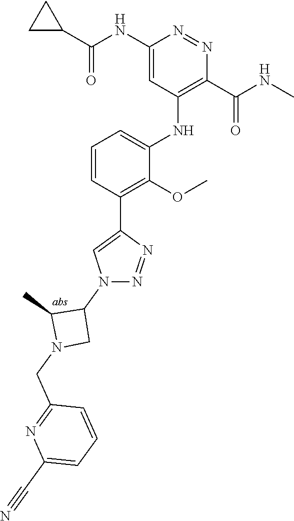 [SMILES: CNC(=O)c1nnc(NC(=O)C2CC2)cc1Nc1cccc(-c2cn(C3CN(Cc4cccc(C#N)n4)[C@H]3C)nn2)c1OC] | A | | C | C |
| 235 | 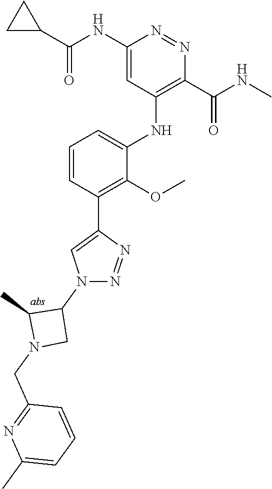 | A | | C | C |
| 236 | 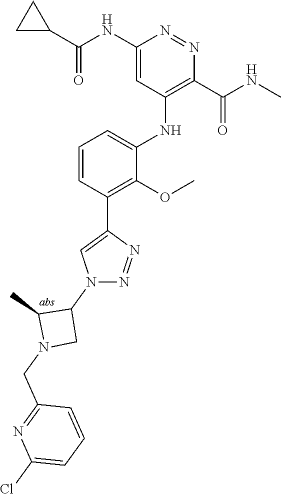 | A | | C | C |
| 237 | 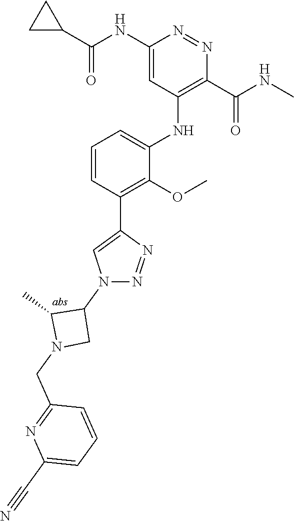 [SMILES: CNC(=O)c1nnc(NC(=O)C2CC2)cc1Nc1cccc(-c2cn(C3CN(Cc4cccc(C#N)n4)[C@@H]3C)nn2)c1OC] | A | | D | D |
| 238 | 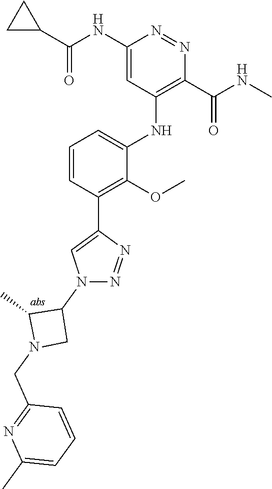 | A | | D | D |
| 239 | 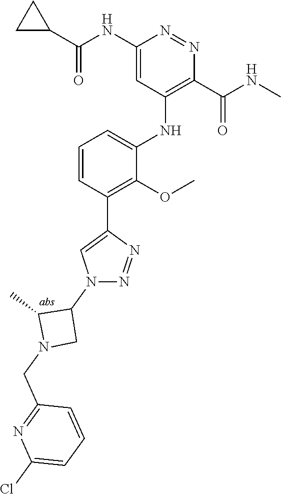 | A | | D | D |
| 240 | 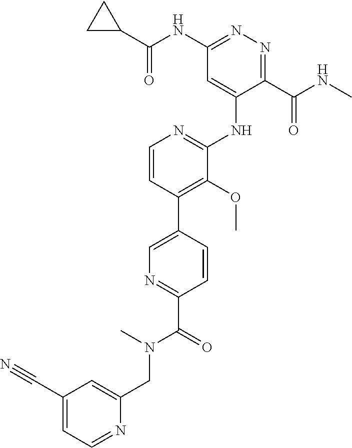 [SMILES: CNC(=O)c1nnc(NC(=O)C2CC2)cc1Nc1nccc(-c2ccc(C(=O)N(C)Cc3cc(C#N)ccn3)nc2)c1OC] | A | | C | D |
| 241 | 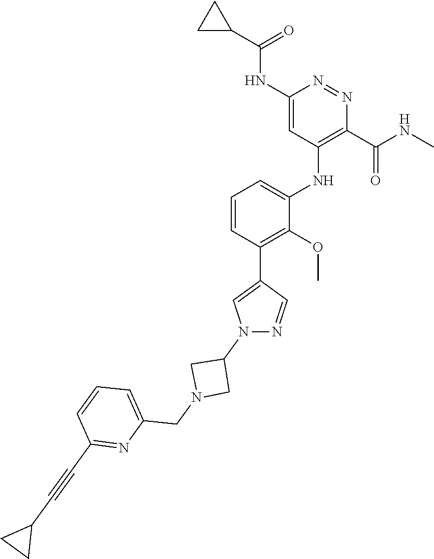 [SMILES: CNC(=O)c1nnc(NC(=O)C2CC2)cc1Nc1cccc(-c2cnn(C3CN(Cc4cccc(C#CC5CC5)n4)C3)c2)c1OC] | A | | C | C |
| 242 | 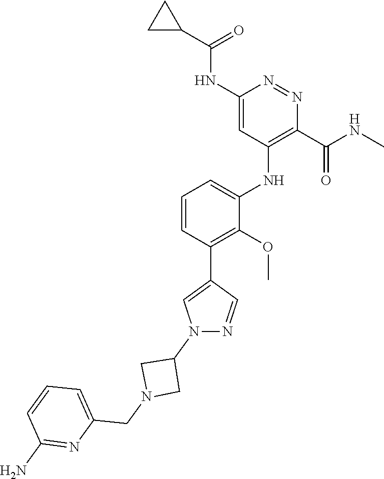 [SMILES: CNC(=O)c1nnc(NC(=O)C2CC2)cc1Nc1cccc(-c2cnn(C3CN(Cc4cccc(N)n4)C3)c2)c1OC] | A | | C | C |
| 243 | 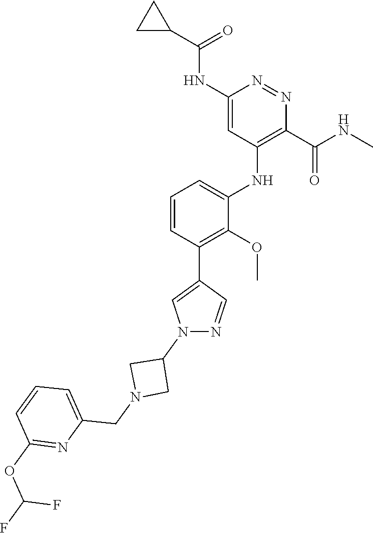 [SMILES: CNC(=O)c1nnc(NC(=O)C2CC2)cc1Nc1cccc(-c2cnn(C3CN(Cc4cccc(OC(F)F)n4)C3)c2)c1OC] | A | | C | C |
| 244 | 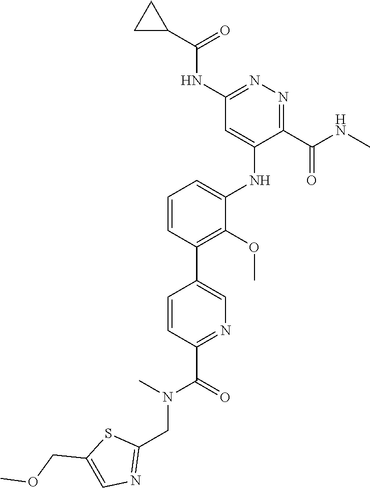 | A | | C | C |
| 245 | 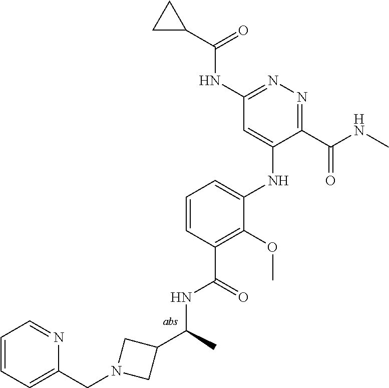 [SMILES: CNC(=O)c1nnc(NC(=O)C2CC2)cc1Nc1cccc(C(=O)N[C@@H](C)C2CN(Cc3ccccn3)C2)c1OC] | C | | E | E |
| 246 | 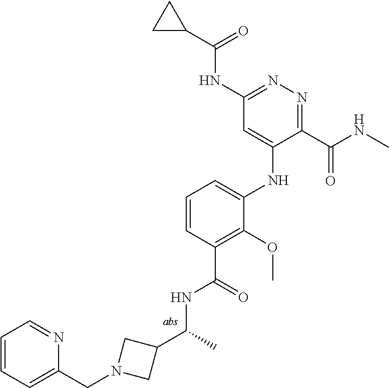 [SMILES: CNC(=O)c1nnc(NC(=O)C2CC2)cc1Nc1cccc(C(=O)N[C@H](C)C2CN(Cc3ccccn3)C2)c1OC] | C | | E | E |
| 247 | 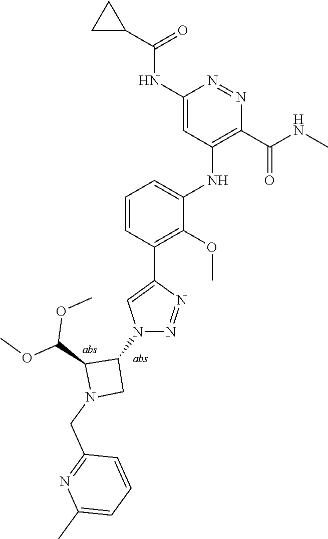 | A | | D | D |
| 248 | 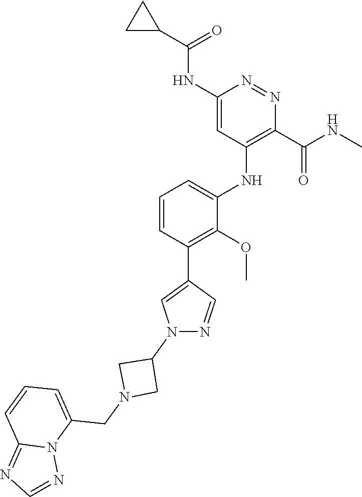 [SMILES: CNC(=O)c1nnc(NC(=O)C2CC2)cc1Nc1cccc(-c2cnn(C3CN(Cc4cccc5ncnn45)C3)c2)c1OC] | A | | C | C |
| 249 | 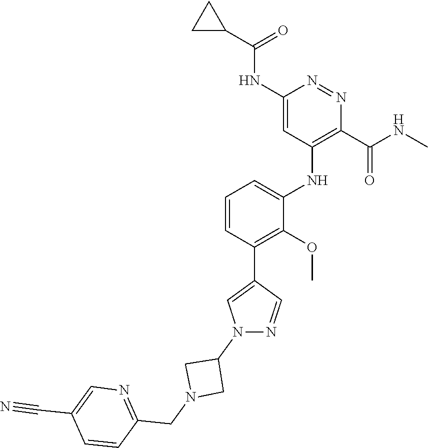 [SMILES: CNC(=O)c1nnc(NC(=O)C2CC2)cc1Nc1cccc(-c2cnn(C3CN(Cc4ccc(C#N)cn4)C3)c2)c1OC] | B | | E | E |
| 250 | 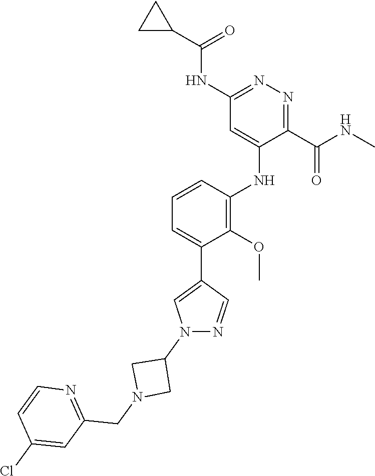 [SMILES: CNC(=O)c1nnc(NC(=O)C2CC2)cc1Nc1cccc(-c2cnn(C3CN(Cc4cc(Cl)ccn4)C3)c2)c1OC] | A | | C | C |
| 251 | 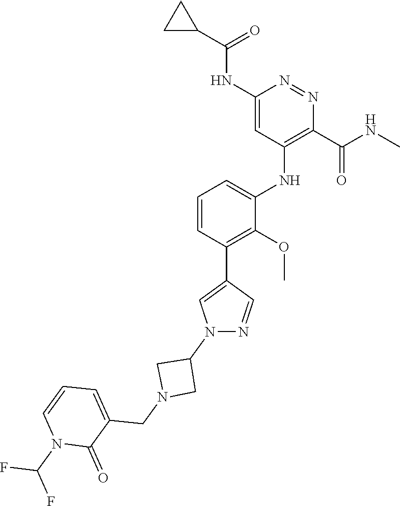 [SMILES: CNC(=O)c1nnc(NC(=O)C2CC2)cc1Nc1cccc(-c2cnn(C3CN(Cc4cccn(C(F)F)c4=O)C3)c2)c1OC] | A | | C | C |
| 252 | 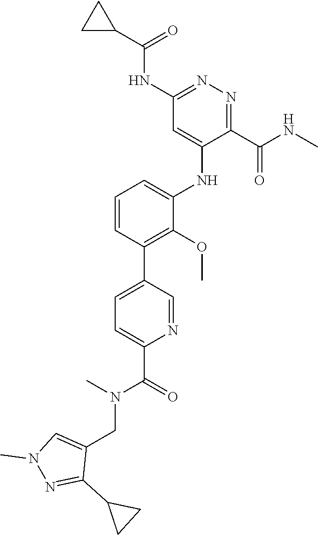 | A | | C | C |
| 253 | 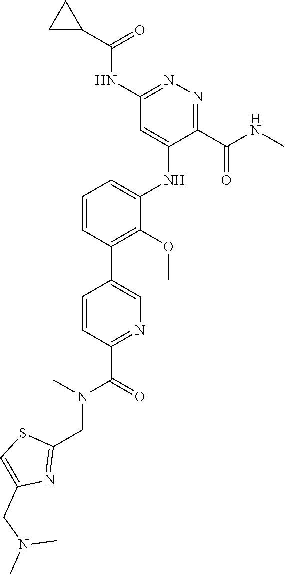 [SMILES: CNC(=O)c1nnc(NC(=O)C2CC2)cc1Nc1cccc(-c2ccc(C(=O)N(C)Cc3nc(CN(C)C)cs3)nc2)c1OC] | B | | E | D |
| 254 | 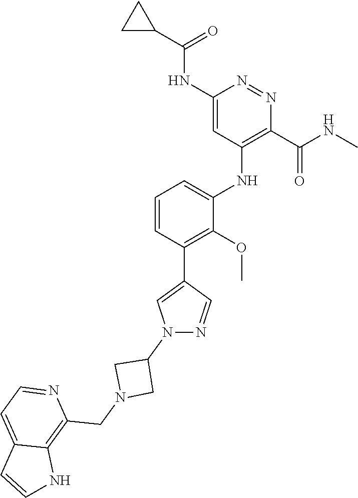 [SMILES: CNC(=O)c1nnc(NC(=O)C2CC2)cc1Nc1cccc(-c2cnn(C3CN(Cc4nccc5cc[nH]c45)C3)c2)c1OC] | A | | C | C |
| 255 | 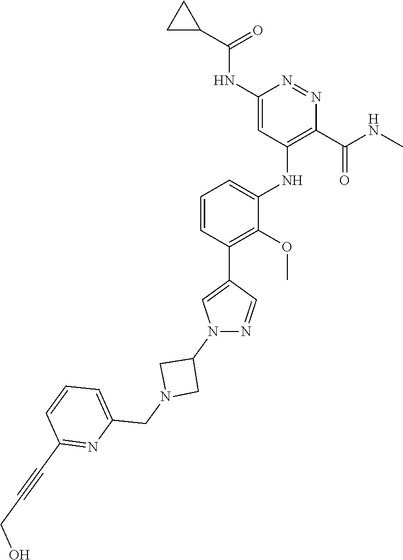 [SMILES: CNC(=O)c1nnc(NC(=O)C2CC2)cc1Nc1cccc(-c2cnn(C3CN(Cc4cccc(C#CCO)n4)C3)c2)c1OC] | A | | C | C |
| 256 | 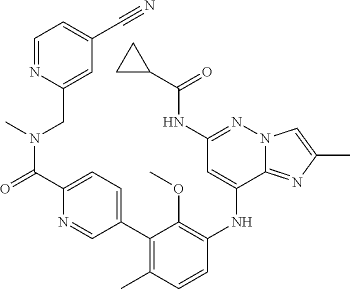 [SMILES: COc1c(Nc2cc(NC(=O)C3CC3)nn3cc(C)nc23)ccc(C)c1-c1ccc(C(=O)N(C)Cc2cc(C#N)ccn2)nc1] | A | | D | D |
| 257 | 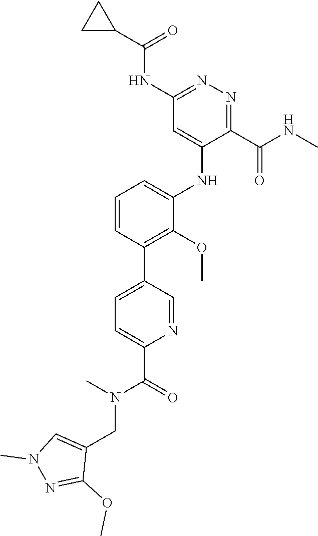 | A | | C | C |
| 258 | 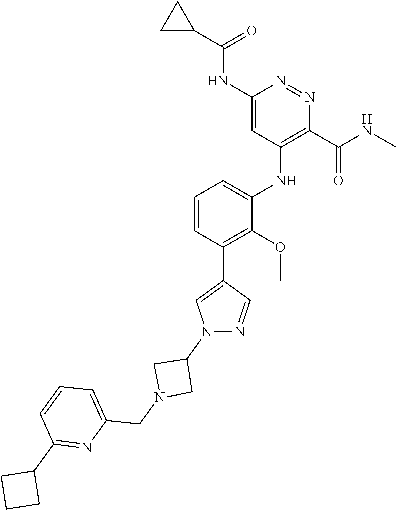 [SMILES: CNC(=O)c1nnc(NC(=O)C2CC2)cc1Nc1cccc(-c2cnn(C3CN(Cc4cccc(C5CCC5)n4)C3)c2)c1OC] | A | | C | C |
| 259 | 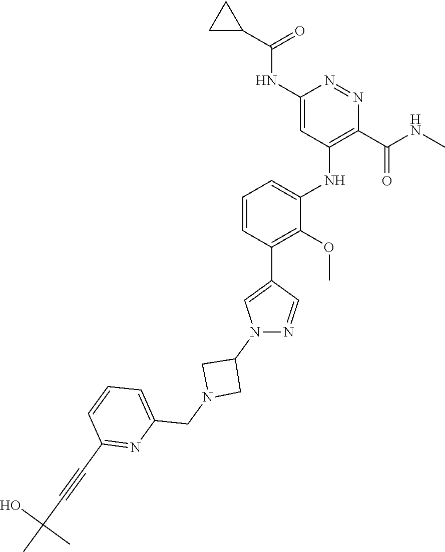 [SMILES: CNC(=O)c1nnc(NC(=O)C2CC2)cc1Nc1cccc(-c2cnn(C3CN(Cc4cccc(C#CC(C)(C)O)n4)C3)c2)c1OC] | A | | C | C |
| 260 | 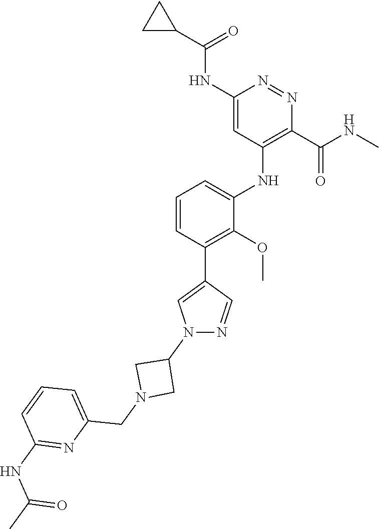 [SMILES: CNC(=O)c1nnc(NC(=O)C2CC2)cc1Nc1cccc(-c2cnn(C3CN(Cc4cccc(NC(C)=O)n4)C3)c2)c1OC] | A | | C | C |
| 261 | 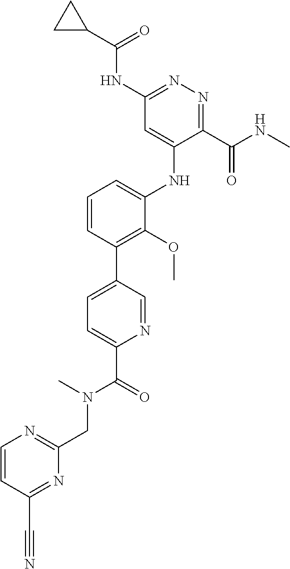 [SMILES: CNC(=O)c1nnc(NC(=O)C2CC2)cc1Nc1cccc(-c2ccc(C(=O)N(C)Cc3nccc(C#N)n3)nc2)c1OC] | A | | C | D |
| 262 | 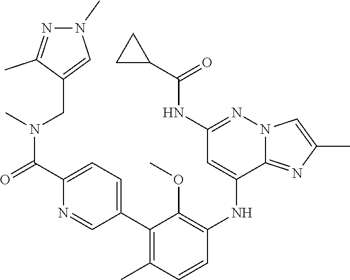 | A | | D | E |
| 263 | 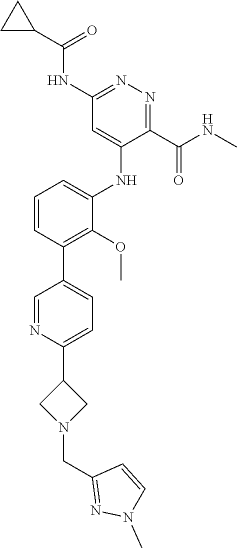 [SMILES: CNC(=O)c1nnc(NC(=O)C2CC2)cc1Nc1cccc(-c2ccc(C3CN(Cc4ccn(C)n4)C3)nc2)c1OC] | B | | E | E |
| 264 | 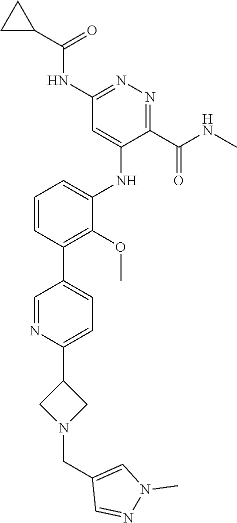 [SMILES: CNC(=O)c1nnc(NC(=O)C2CC2)cc1Nc1cccc(-c2ccc(C3CN(Cc4cnn(C)c4)C3)nc2)c1OC] | C | | E | E |
| 265 | 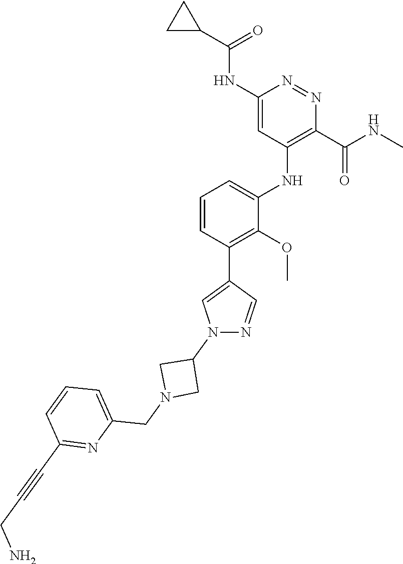 [SMILES: CNC(=O)c1nnc(NC(=O)C2CC2)cc1Nc1cccc(-c2cnn(C3CN(Cc4cccc(C#CCN)n4)C3)c2)c1OC] | A | | D | D |
| 266 | 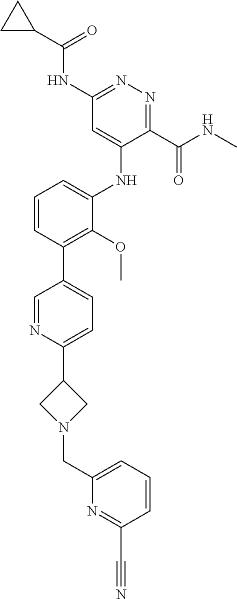 [SMILES: CNC(=O)c1nnc(NC(=O)C2CC2)cc1Nc1cccc(-c2ccc(C3CN(Cc4cccc(C#N)n4)C3)nc2)c1OC] | A | | D | D |
| 267 | 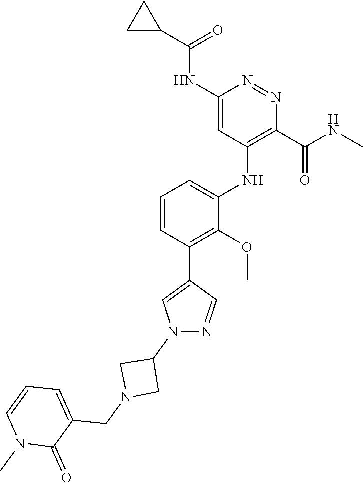 | A | | C | C |
| 268 | 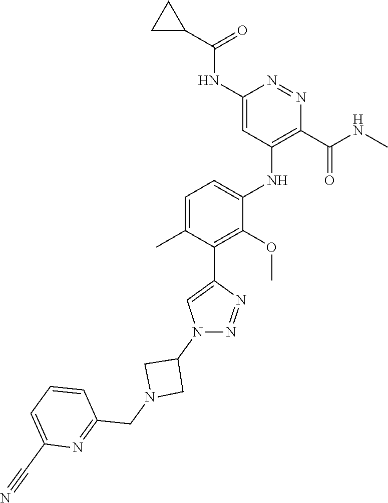 [SMILES: CNC(=O)c1nnc(NC(=O)C2CC2)cc1Nc1ccc(C)c(-c2cn(C3CN(Cc4cccc(C#N)n4)C3)nn2)c1OC] | A | | D | D |
| 269 | 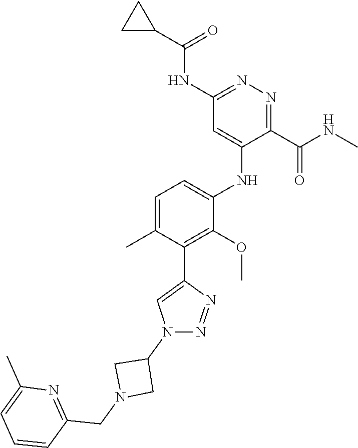 [SMILES: CNC(=O)c1nnc(NC(=O)C2CC2)cc1Nc1ccc(C)c(-c2cn(C3CN(Cc4cccc(C)n4)C3)nn2)c1OC] | A | | D | D |
| 270 | 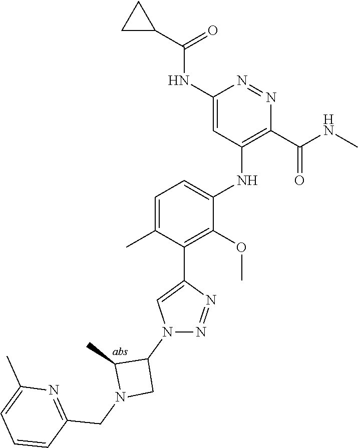 [SMILES: CNC(=O)c1nnc(NC(=O)C2CC2)cc1Nc1ccc(C)c(-c2cn(C3CN(Cc4cccc(C)n4)[C@H]3C)nn2)c1OC] | A | | D | D |
| 271 | 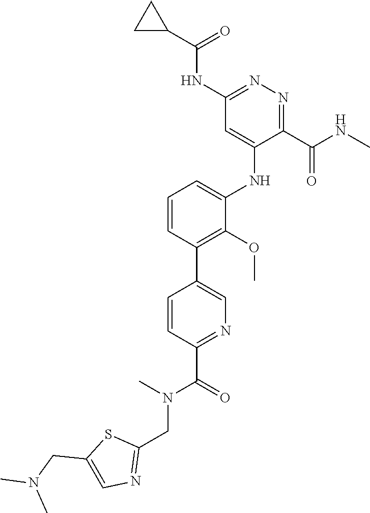 | A | | D | D |
| 272 | 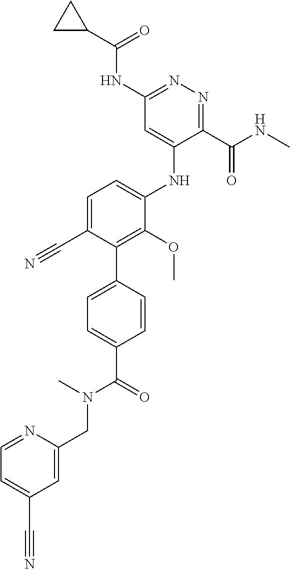 [SMILES: CNC(=O)c1nnc(NC(=O)C2CC2)cc1Nc1ccc(C#N)c(-c2ccc(C(=O)N(C)Cc3cc(C#N)ccn3)cc2)c1OC] | A | | D | D |
| 273 | 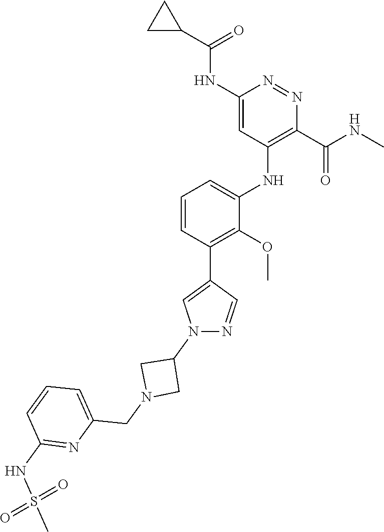 [SMILES: CNC(=O)c1nnc(NC(=O)C2CC2)cc1Nc1cccc(-c2cnn(C3CN(Cc4cccc(NS(C)(=O)=O)n4)C3)c2)c1OC] | A | | D | D |
| 274 | 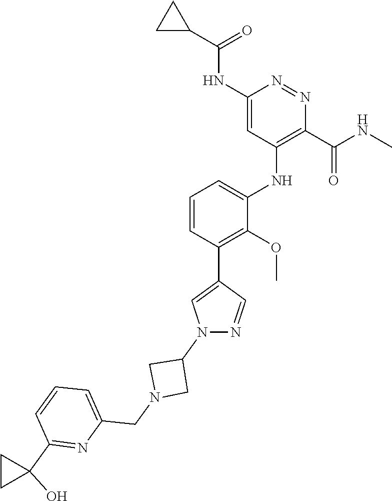 [SMILES: CNC(=O)c1nnc(NC(=O)C2CC2)cc1Nc1cccc(-c2cnn(C3CN(Cc4cccc(C5(O)CC5)n4)C3)c2)c1OC] | A | | C | C |
| 275 | 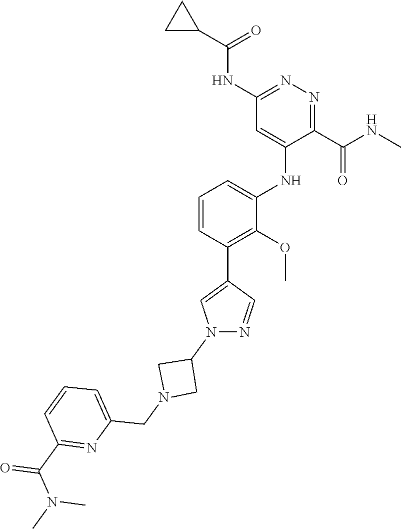 [SMILES: CNC(=O)c1nnc(NC(=O)C2CC2)cc1Nc1cccc(-c2cnn(C3CN(Cc4cccc(C(=O)N(C)C)n4)C3)c2)c1OC] | A | | B | B |
| 276 | 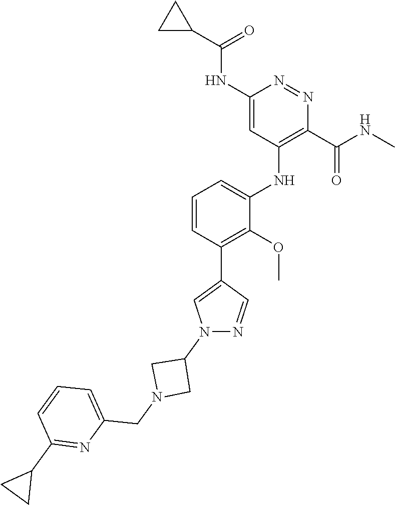 [SMILES: CNC(=O)c1nnc(NC(=O)C2CC2)cc1Nc1cccc(-c2cnn(C3CN(Cc4cccc(C5CC5)n4)C3)c2)c1OC] | A | | C | C |
| 277 | 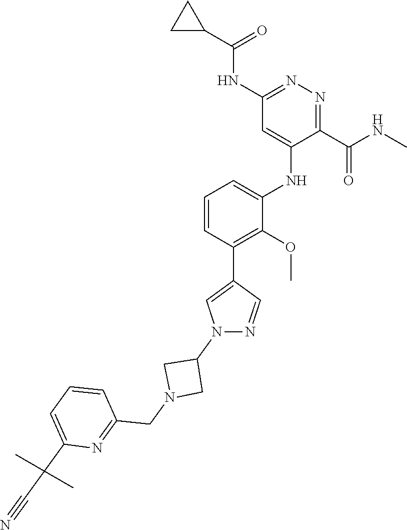 [SMILES: CNC(=O)c1nnc(NC(=O)C2CC2)cc1Nc1cccc(-c2cnn(C3CN(Cc4cccc(C(C)(C)C#N)n4)C3)c2)c1OC] | A | | C | C |
| 278 | 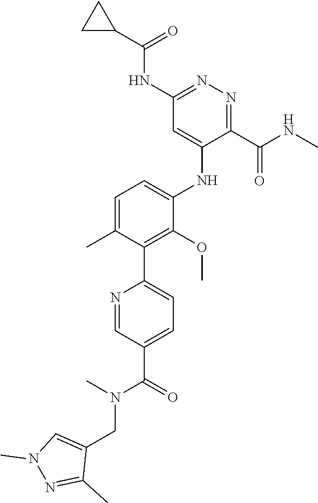 | A | | D | D |
| 279 | 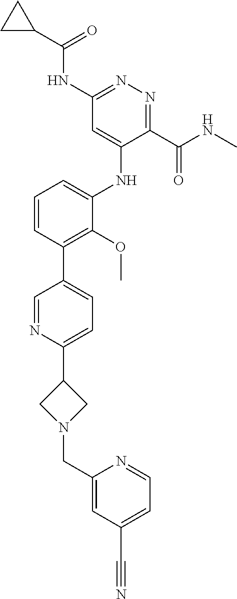 [SMILES: CNC(=O)c1nnc(NC(=O)C2CC2)cc1Nc1cccc(-c2ccc(C3CN(Cc4cc(C#N)ccn4)C3)nc2)c1OC] | B | | E | E |
| 280 | 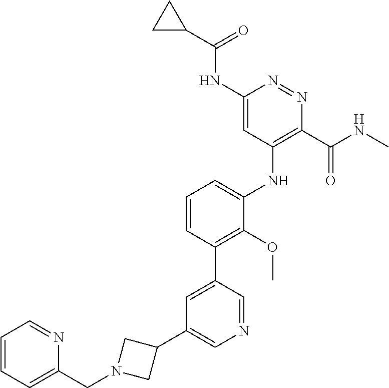 [SMILES: CNC(=O)c1nnc(NC(=O)C2CC2)cc1Nc1cccc(-c2cncc(C3CN(Cc4ccccn4)C3)c2)c1OC] | B | | E | E |
| 281 | 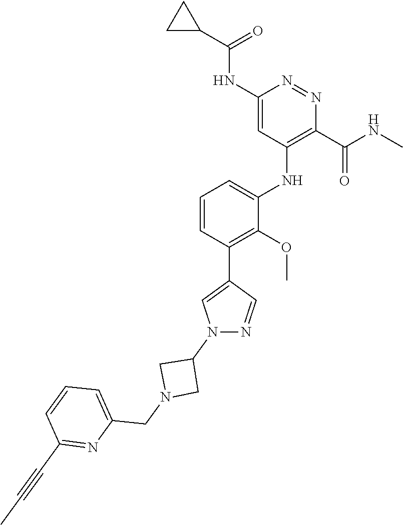 [SMILES: CC#Cc1cccc(CN2CC(n3cc(-c4cccc(Nc5cc(NC(=O)C6CC6)nnc5C(=O)NC)c4OC)cn3)C2)n1] | A | | C | C |
| 282 | 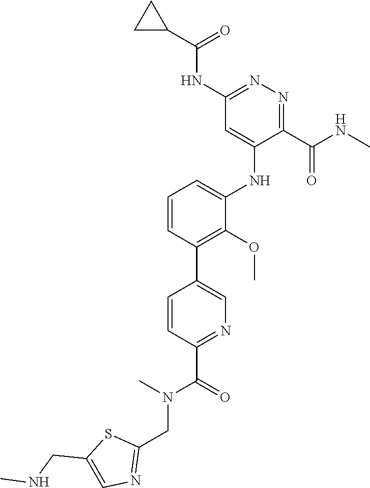 | A | | D | D |
| 283 | 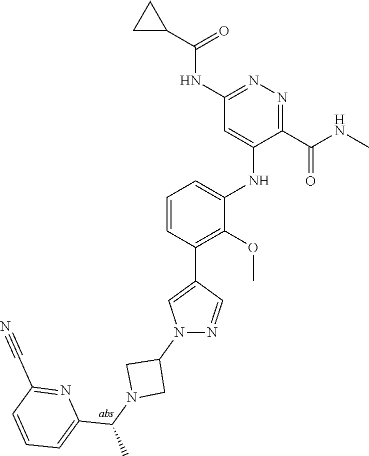 [SMILES: CNC(=O)c1nnc(NC(=O)C2CC2)cc1Nc1cccc(-c2cnn(C3CN([C@H](C)c4cccc(C#N)n4)C3)c2)c1OC] | A | | D | D |
| 284 | 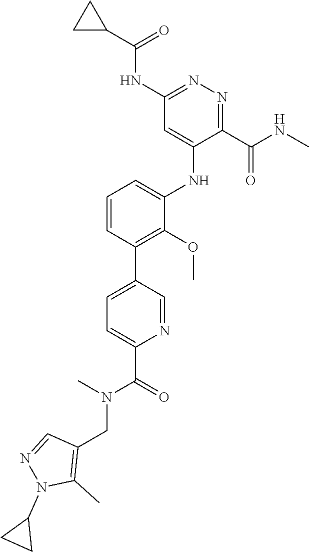 [SMILES: CNC(=O)c1nnc(NC(=O)C2CC2)cc1Nc1cccc(-c2ccc(C(=O)N(C)Cc3cnn(C4CC4)c3C)nc2)c1OC] | A | | D | D |
| 285 | 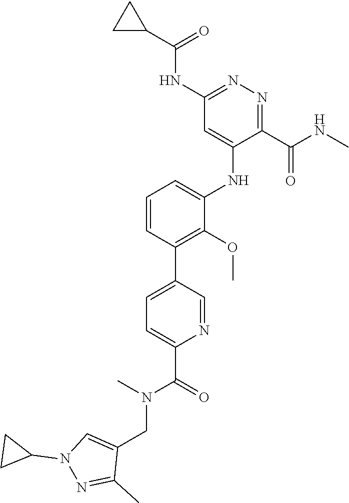 [SMILES: CNC(=O)c1nnc(NC(=O)C2CC2)cc1Nc1cccc(-c2ccc(C(=O)N(C)Cc3cn(C4CC4)nc3C)nc2)c1OC] | A | | D | D |
| 286 | 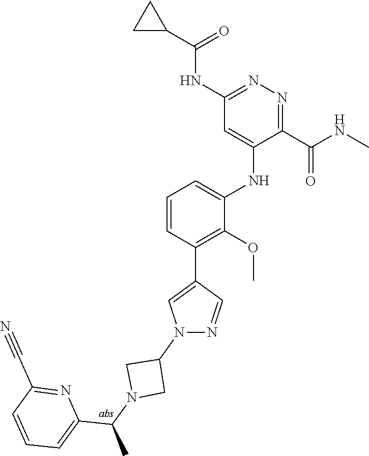 [SMILES: CNC(=O)c1nnc(NC(=O)C2CC2)cc1Nc1cccc(-c2cnn(C3CN([C@@H](C)c4cccc(C#N)n4)C3)c2)c1OC] | A | | C | C |
| 287 | 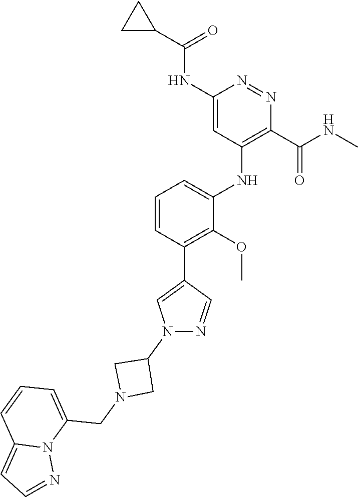 [SMILES: CNC(=O)c1nnc(NC(=O)C2CC2)cc1Nc1cccc(-c2cnn(C3CN(Cc4cccc5ccnn45)C3)c2)c1OC] | A | | C | C |
| 288 | 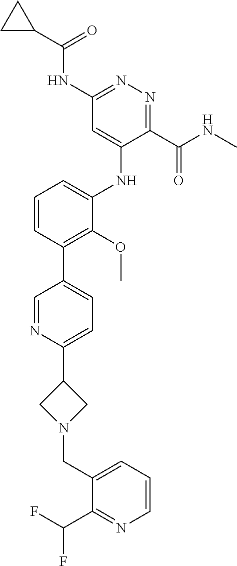 [SMILES: CNC(=O)c1nnc(NC(=O)C2CC2)cc1Nc1cccc(-c2ccc(C3CN(Cc4cccnc4C(F)F)C3)nc2)c1OC] | C | | E | E |
| 289 | 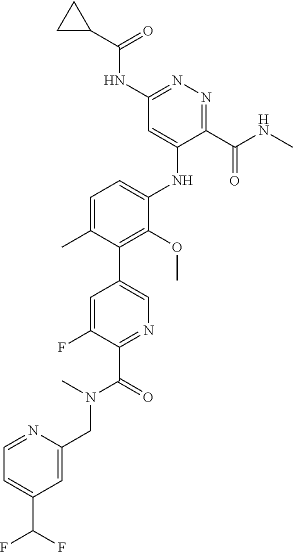 [SMILES: CNC(=O)c1nnc(NC(=O)C2CC2)cc1Nc1ccc(C)c(-c2cnc(C(=O)N(C)Cc3cc(C(F)F)ccn3)c(F)c2)c1OC] | A | | C | C |
| 290 | 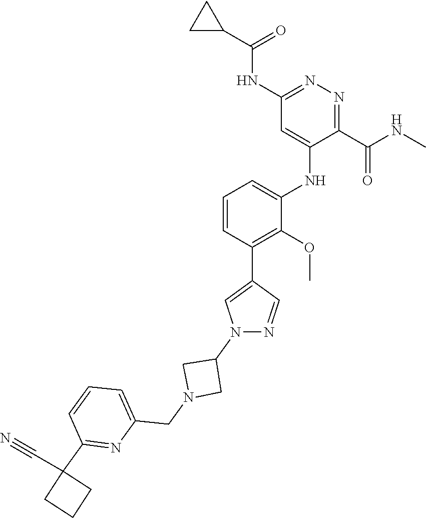 [SMILES: CNC(=O)c1nnc(NC(=O)C2CC2)cc1Nc1cccc(-c2cnn(C3CN(Cc4cccc(C5(C#N)CCC5)n4)C3)c2)c1OC] | A | | C | C |
| 291 | 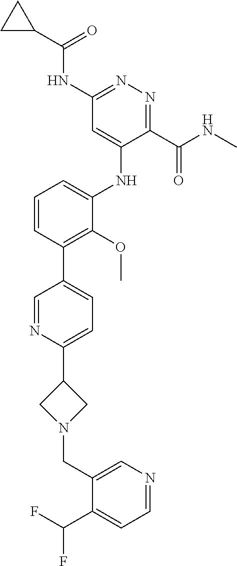 [SMILES: CNC(=O)c1nnc(NC(=O)C2CC2)cc1Nc1cccc(-c2ccc(C3CN(Cc4cnccc4C(F)F)C3)nc2)c1OC] | B | | E | D |
| 292 | 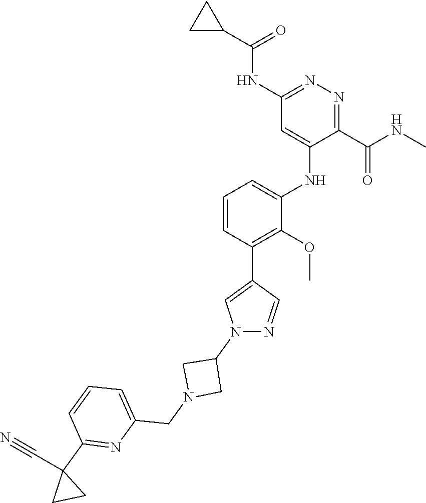 [SMILES: CNC(=O)c1nnc(NC(=O)C2CC2)cc1Nc1cccc(-c2cnn(C3CN(Cc4cccc(C5(C#N)CC5)n4)C3)c2)c1OC] | A | | C | C |
| 293 | 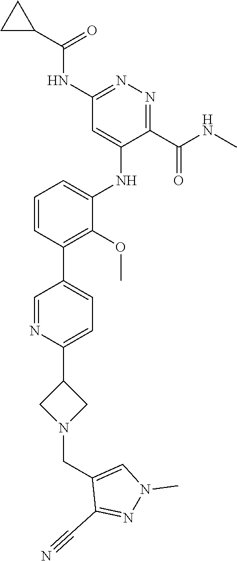 [SMILES: CNC(=O)c1nnc(NC(=O)C2CC2)cc1Nc1cccc(-c2ccc(C3CN(Cc4cn(C)nc4C#N)C3)nc2)c1OC] | C | | D | E |
| 294 | 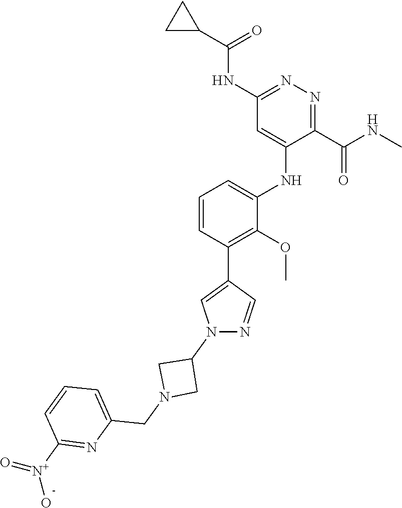 | A | | C | C |
| 295 | 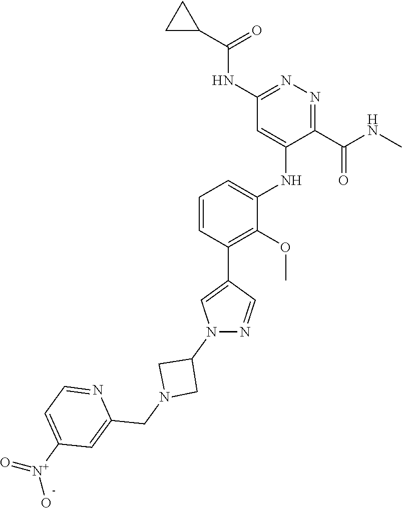 | A | | C | C |
| 296 | 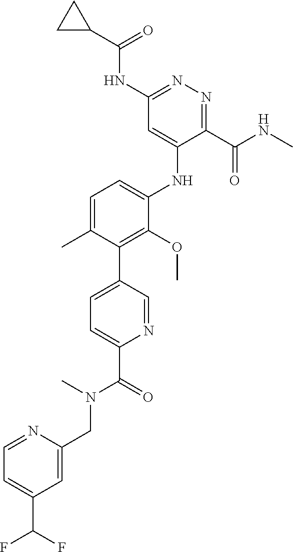 [SMILES: CNC(=O)c1nnc(NC(=O)C2CC2)cc1Nc1ccc(C)c(-c2ccc(C(=O)N(C)Cc3cc(C(F)F)ccn3)nc2)c1OC] | A | | C | C |
| 297 | 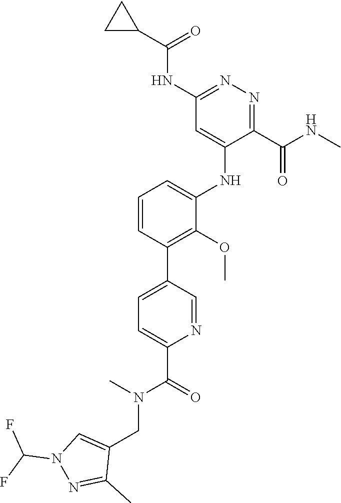 [SMILES: CNC(=O)c1nnc(NC(=O)C2CC2)cc1Nc1cccc(-c2ccc(C(=O)N(C)Cc3cn(C(F)F)nc3C)nc2)c1OC] | A | | C | C |
| 298 | 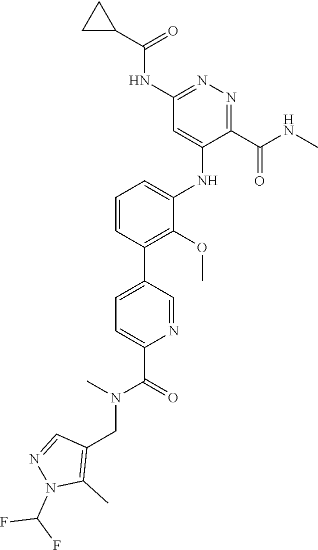 [SMILES: CNC(=O)c1nnc(NC(=O)C2CC2)cc1Nc1cccc(-c2ccc(C(=O)N(C)Cc3cnn(C(F)F)c3C)nc2)c1OC] | A | | D | D |
| 299 | 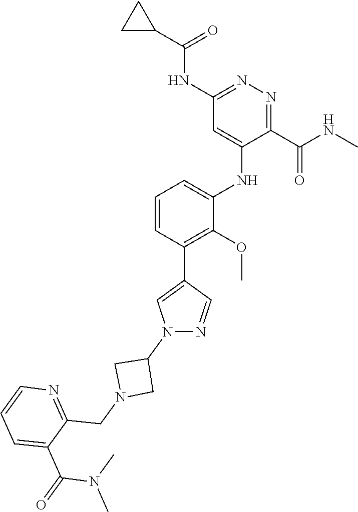 [SMILES: CNC(=O)c1nnc(NC(=O)C2CC2)cc1Nc1cccc(-c2cnn(C3CN(Cc4ncccc4C(=O)N(C)C)C3)c2)c1OC] | A | | D | D |
| 300 | 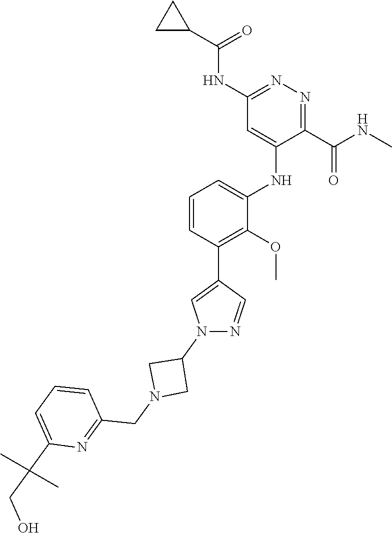 [SMILES: CNC(=O)c1nnc(NC(=O)C2CC2)cc1Nc1cccc(-c2cnn(C3CN(Cc4cccc(C(C)(C)CO)n4)C3)c2)c1OC] | A | | C | C |
| 301 | 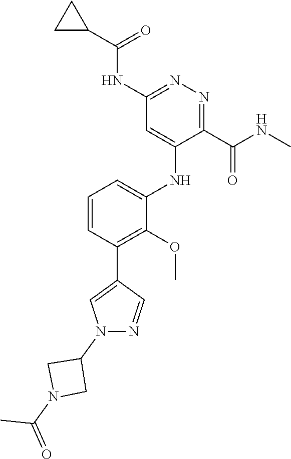 [SMILES: CNC(=O)c1nnc(NC(=O)C2CC2)cc1Nc1cccc(-c2cnn(C3CN(C(C)=O)C3)c2)c1OC] | C | E | E | E |
| 302 | 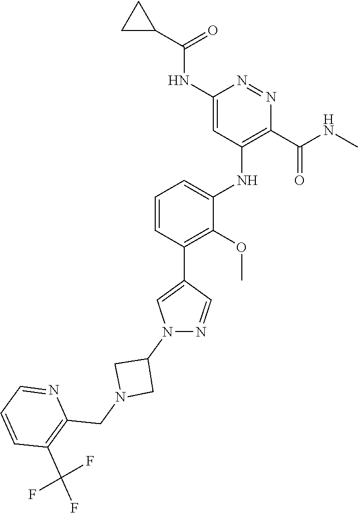 [SMILES: CNC(=O)c1nnc(NC(=O)C2CC2)cc1Nc1cccc(-c2cnn(C3CN(Cc4ncccc4C(F)(F)F)C3)c2)c1OC] | A | | D | D |
| 303 | 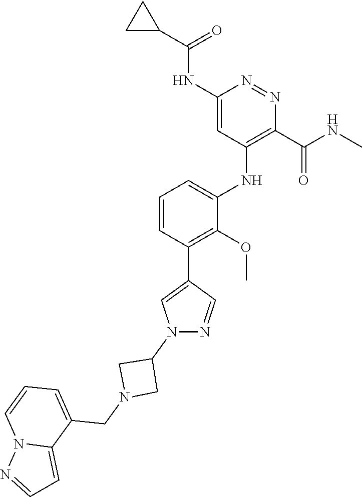 [SMILES: CNC(=O)c1nnc(NC(=O)C2CC2)cc1Nc1cccc(-c2cnn(C3CN(Cc4cccn5nccc45)C3)c2)c1OC] | A | | C | C |
| 304 | 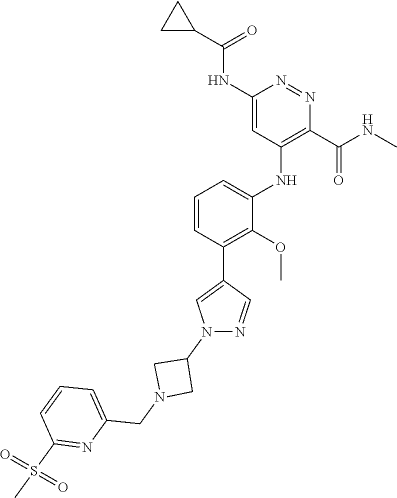 [SMILES: CNC(=O)c1nnc(NC(=O)C2CC2)cc1Nc1cccc(-c2cnn(C3CN(Cc4cccc(S(C)(=O)=O)n4)C3)c2)c1OC] | A | | C | C |
| 305 | 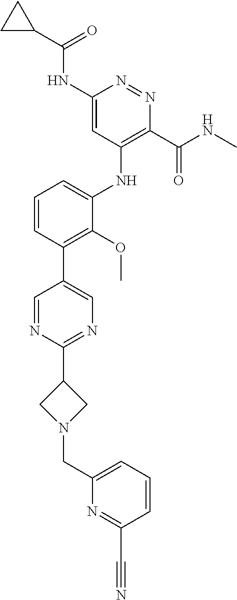 [SMILES: CNC(=O)c1nnc(NC(=O)C2CC2)cc1Nc1cccc(-c2cnc(C3CN(Cc4cccc(C#N)n4)C3)nc2)c1OC] | A | | D | D |
| 306 | 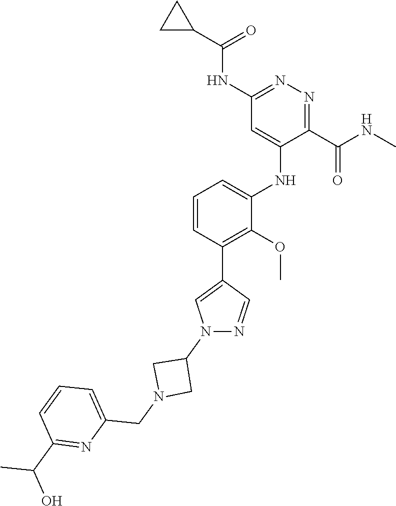 [SMILES: CNC(=O)c1nnc(NC(=O)C2CC2)cc1Nc1cccc(-c2cnn(C3CN(Cc4cccc(C(C)O)n4)C3)c2)c1OC] | A | | C | C |
| 307 | 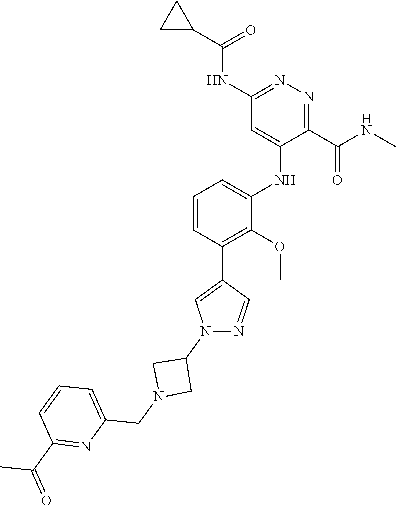 [SMILES: CNC(=O)c1nnc(NC(=O)C2CC2)cc1Nc1cccc(-c2cnn(C3CN(Cc4cccc(C(C)=O)n4)C3)c2)c1OC] | A | | C | C |
| 308 | 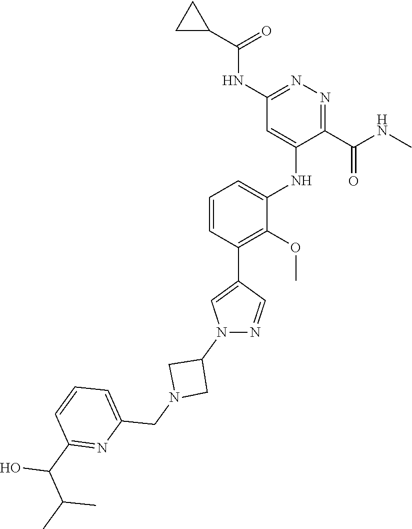 [SMILES: CNC(=O)c1nnc(NC(=O)C2CC2)cc1Nc1cccc(-c2cnn(C3CN(Cc4cccc(C(O)C(C)C)n4)C3)c2)c1OC] | B | | C | C |
| 309 | 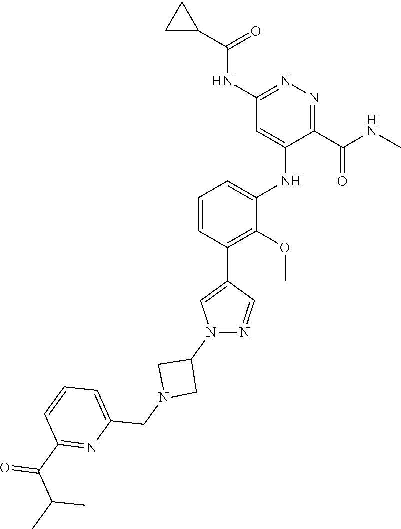 [SMILES: CNC(=O)c1nnc(NC(=O)C2CC2)cc1Nc1cccc(-c2cnn(C3CN(Cc4cccc(C(=O)C(C)C)n4)C3)c2)c1OC] | A | | D | C |
| 310 | 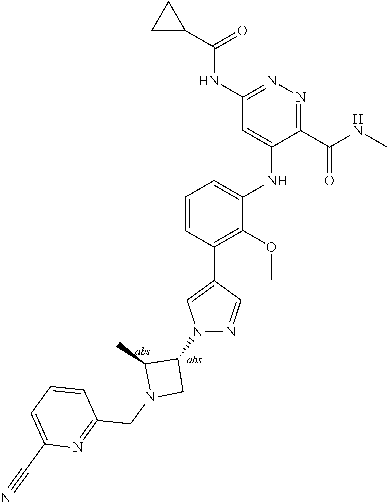 [SMILES: CNC(=O)c1nnc(NC(=O)C2CC2)cc1Nc1cccc(-c2cnn([C@@H]3CN(Cc4cccc(C#N)n4)[C@H]3C)c2)c1OC] | A | | D | D |
| 311 | 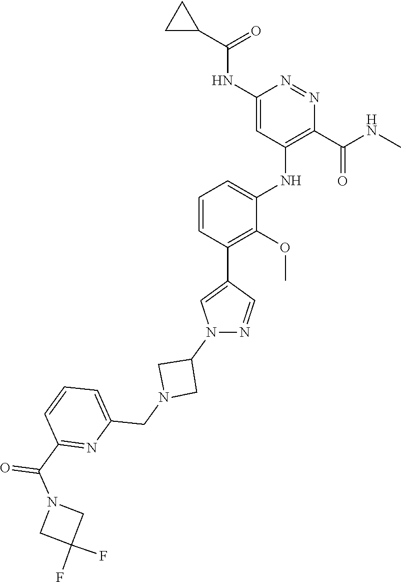 [SMILES: CNC(=O)c1nnc(NC(=O)C2CC2)cc1Nc1cccc(-c2cnn(C3CN(Cc4cccc(C(=O)N5CC(F)(F)C5)n4)C3)c2)c1OC] | A | | C | C |
| 312 | 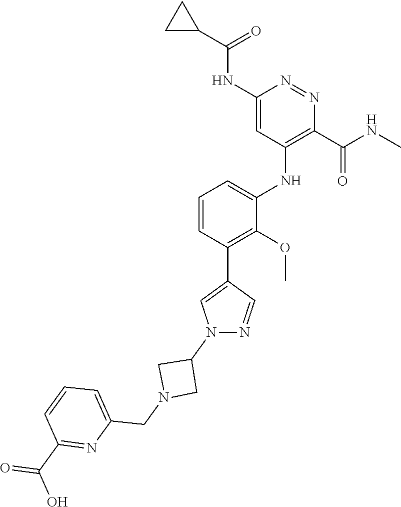 [SMILES: CNC(=O)c1nnc(NC(=O)C2CC2)cc1Nc1cccc(-c2cnn(C3CN(Cc4cccc(C(=O)O)n4)C3)c2)c1OC] | A | | E | E |
| 313 | 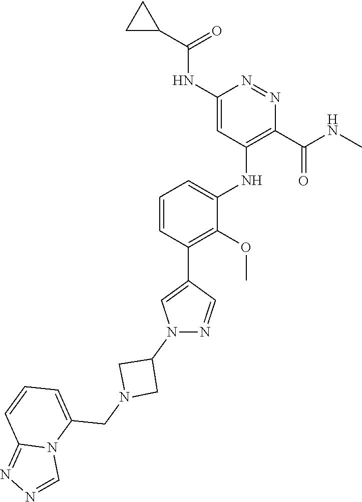 [SMILES: CNC(=O)c1nnc(NC(=O)C2CC2)cc1Nc1cccc(-c2cnn(C3CN(Cc4cccc5nncn45)C3)c2)c1OC] | B | | D | D |
| 314 | 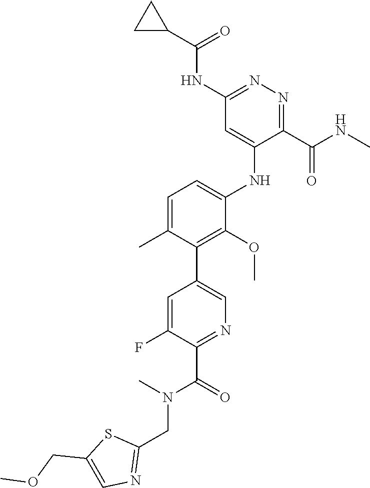 | A | | D | C |
| 315 | 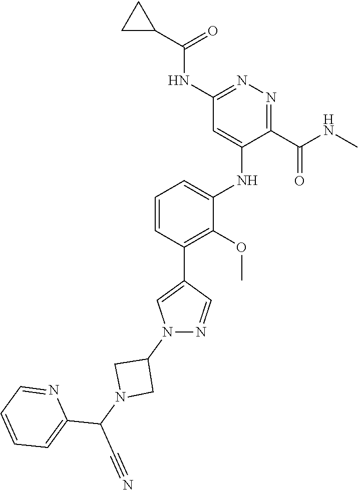 [SMILES: CNC(=O)c1nnc(NC(=O)C2CC2)cc1Nc1cccc(-c2cnn(C3CN(C(C#N)c4ccccn4)C3)c2)c1OC] | A | | D | E |
| 316 | 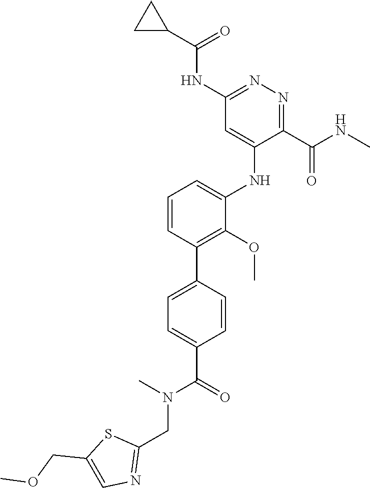 | A | | D | D |
| 317 | 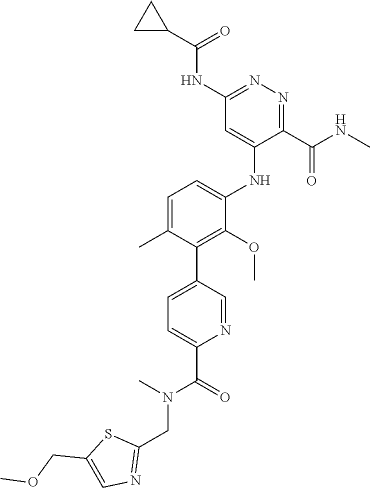 | A | | D | C |
| 318 | 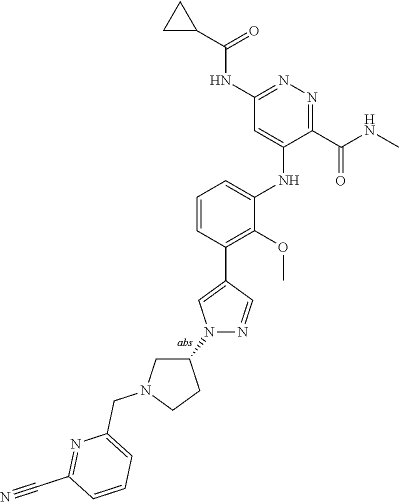 [SMILES: CNC(=O)c1nnc(NC(=O)C2CC2)cc1Nc1cccc(-c2cnn([C@@H]3CCN(Cc4cccc(C#N)n4)C3)c2)c1OC] | A | | D | D |
| 319 | 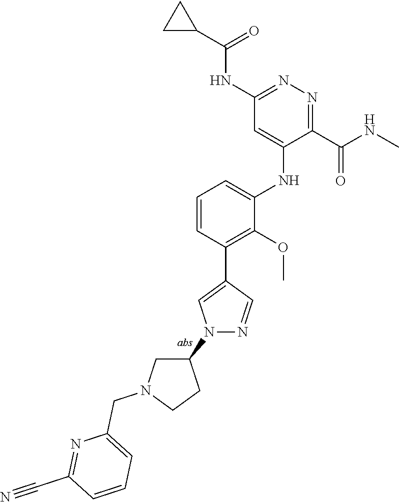 [SMILES: CNC(=O)c1nnc(NC(=O)C2CC2)cc1Nc1cccc(-c2cnn([C@H]3CCN(Cc4cccc(C#N)n4)C3)c2)c1OC] | A | | C | C |
| 320 | 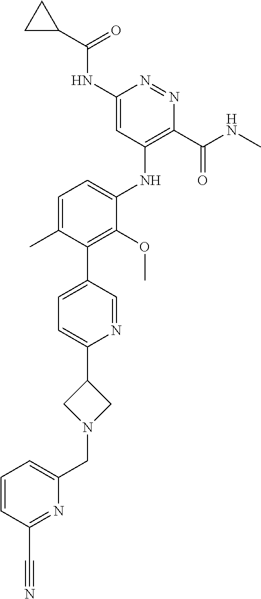 [SMILES: CNC(=O)c1nnc(NC(=O)C2CC2)cc1Nc1ccc(C)c(-c2ccc(C3CN(Cc4cccc(C#N)n4)C3)nc2)c1OC] | A | | D | D |
| 321 | 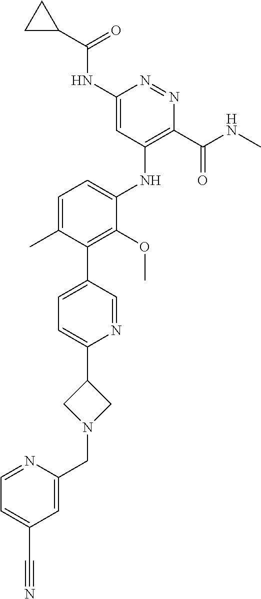 [SMILES: CNC(=O)c1nnc(NC(=O)C2CC2)cc1Nc1ccc(C)c(-c2ccc(C3CN(Cc4cc(C#N)ccn4)C3)nc2)c1OC] | B | | E | E |
| 322 | 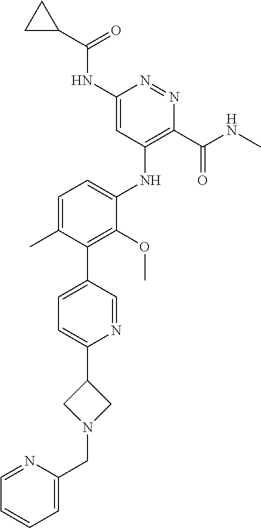 [SMILES: CNC(=O)c1nnc(NC(=O)C2CC2)cc1Nc1ccc(C)c(-c2ccc(C3CN(Cc4ccccn4)C3)nc2)c1OC] | B | | E | E |
| 323 | 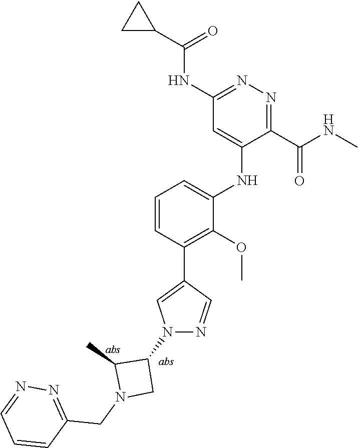 [SMILES: CNC(=O)c1nnc(NC(=O)C2CC2)cc1Nc1cccc(-c2cnn([C@@H]3CN(Cc4cccnn4)[C@H]3C)c2)c1OC] | A | | D | D |
| 324 | 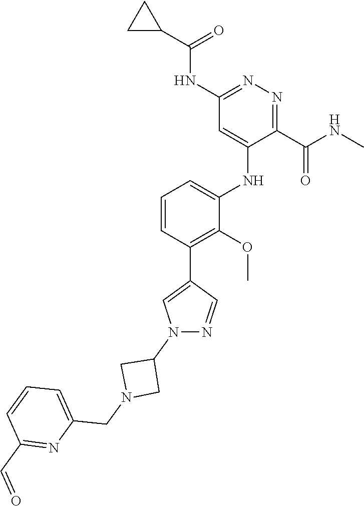 [SMILES: CNC(=O)c1nnc(NC(=O)C2CC2)cc1Nc1cccc(-c2cnn(C3CN(Cc4cccc(C=O)n4)C3)c2)c1OC] | A | | C | C |
| 325 | 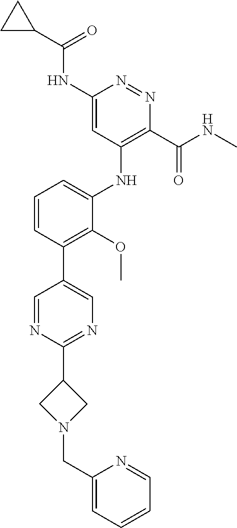 [SMILES: CNC(=O)c1nnc(NC(=O)C2CC2)cc1Nc1cccc(-c2cnc(C3CN(Cc4ccccn4)C3)nc2)c1OC] | A | | E | E |
| 326 | 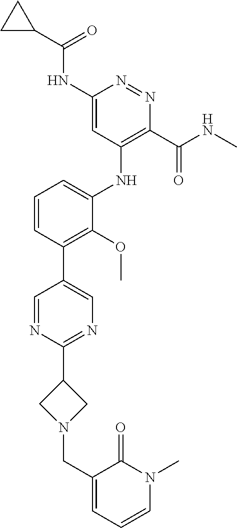 [SMILES: CNC(=O)c1nnc(NC(=O)C2CC2)cc1Nc1cccc(-c2cnc(C3CN(Cc4cccn(C)c4=O)C3)nc2)c1OC] | B | | E | E |
| 327 | 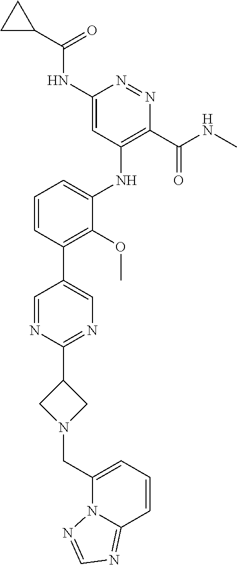 [SMILES: CNC(=O)c1nnc(NC(=O)C2CC2)cc1Nc1cccc(-c2cnc(C3CN(Cc4cccc5ncnn45)C3)nc2)c1OC] | A | | E | E |
| 328 | 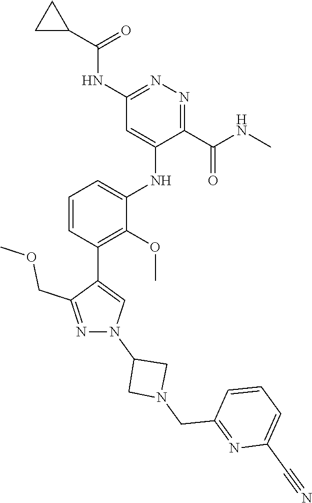 [SMILES: CNC(=O)c1nnc(NC(=O)C2CC2)cc1Nc1cccc(-c2cn(C3CN(Cc4cccc(C#N)n4)C3)nc2COC)c1OC] | A | D | C | C |
| 329 | 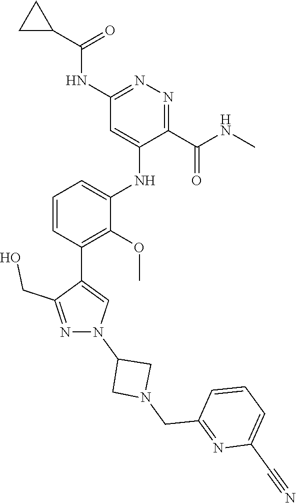 [SMILES: CNC(=O)c1nnc(NC(=O)C2CC2)cc1Nc1cccc(-c2cn(C3CN(Cc4cccc(C#N)n4)C3)nc2CO)c1OC] | A | D | D | D |
| 330 | 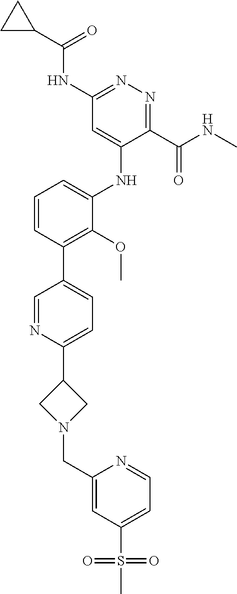 [SMILES: CNC(=O)c1nnc(NC(=O)C2CC2)cc1Nc1cccc(-c2ccc(C3CN(Cc4cc(S(C)(=O)=O)ccn4)C3)nc2)c1OC] | A | | D | D |
| 331 | 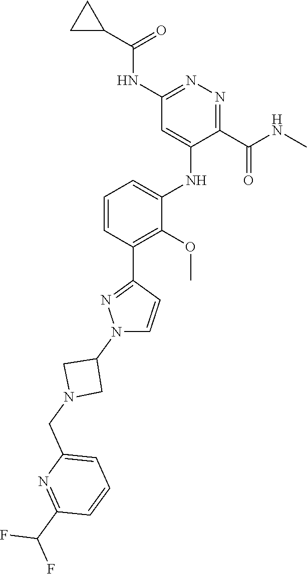 [SMILES: CNC(=O)c1nnc(NC(=O)C2CC2)cc1Nc1cccc(-c2ccn(C3CN(Cc4cccc(C(F)F)n4)C3)n2)c1OC] | A | | D | D |
| 332 | 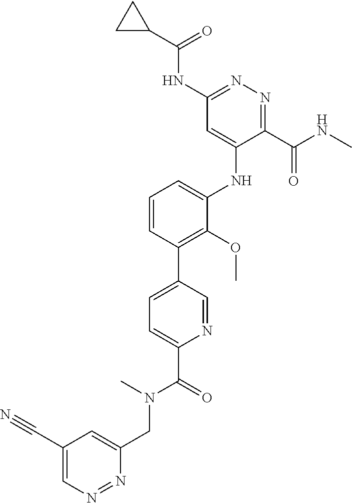 [SMILES: CNC(=O)c1nnc(NC(=O)C2CC2)cc1Nc1cccc(-c2ccc(C(=O)N(C)Cc3cc(C#N)cnn3)nc2)c1OC] | A | | D | D |
| 333 | 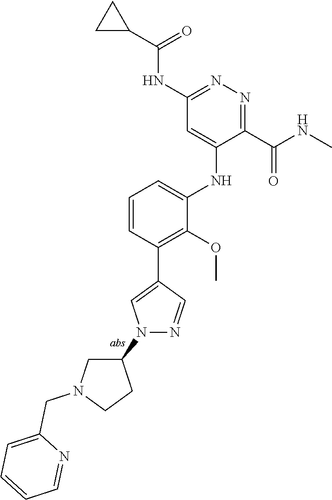 [SMILES: CNC(=O)c1nnc(NC(=O)C2CC2)cc1Nc1cccc(-c2cnn([C@H]3CCN(Cc4ccccn4)C3)c2)c1OC] | A | | D | D |
| 334 | 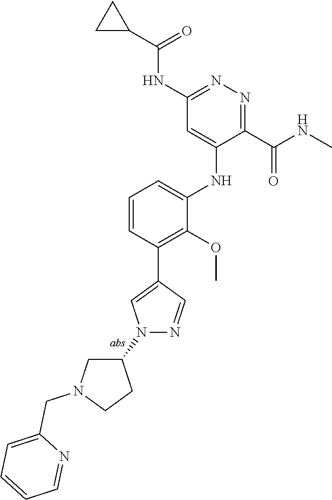 [SMILES: CNC(=O)c1nnc(NC(=O)C2CC2)cc1Nc1cccc(-c2cnn([C@@H]3CCN(Cc4ccccn4)C3)c2)c1OC] | A | | D | D |
| 335 | 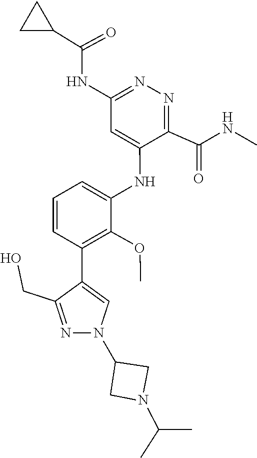 [SMILES: CNC(=O)c1nnc(NC(=O)C2CC2)cc1Nc1cccc(-c2cn(C3CN(C(C)C)C3)nc2CO)c1OC] | C | | E | E |
| 336 | 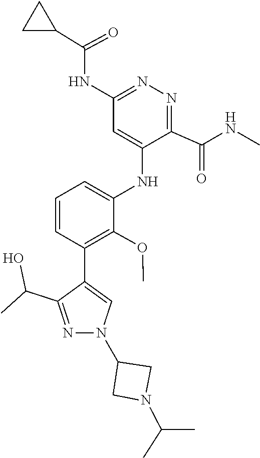 [SMILES: CNC(=O)c1nnc(NC(=O)C2CC2)cc1Nc1cccc(-c2cn(C3CN(C(C)C)C3)nc2C(C)O)c1OC] | C | | E | E |
| 337 | 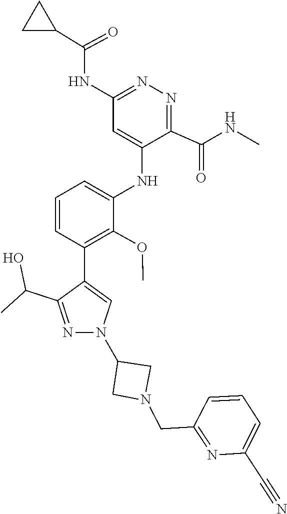 [SMILES: CNC(=O)c1nnc(NC(=O)C2CC2)cc1Nc1cccc(-c2cn(C3CN(Cc4cccc(C#N)n4)C3)nc2C(C)O)c1OC] | A | | E | E |
| 338 | 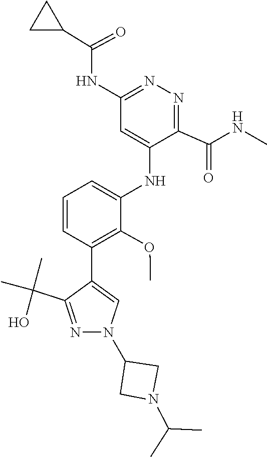 [SMILES: CNC(=O)c1nnc(NC(=O)C2CC2)cc1Nc1cccc(-c2cn(C3CN(C(C)C)C3)nc2C(C)(C)O)c1OC] | C | | D | E |
| 339 | 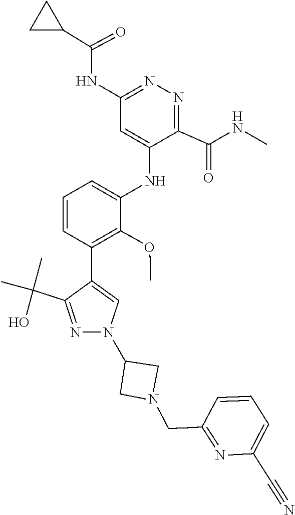 [SMILES: CNC(=O)c1nnc(NC(=O)C2CC2)cc1Nc1cccc(-c2cn(C3CN(Cc4cccc(C#N)n4)C3)nc2C(C)(C)O)c1OC] | C | | E | E |
| 340 | 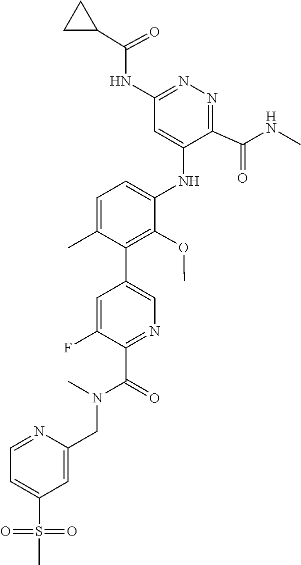 [SMILES: CNC(=O)c1nnc(NC(=O)C2CC2)cc1Nc1ccc(C)c(-c2cnc(C(=O)N(C)Cc3cc(S(C)(=O)=O)ccn3)c(F)c2)c1OC] | A | | C | C |
| 341 | 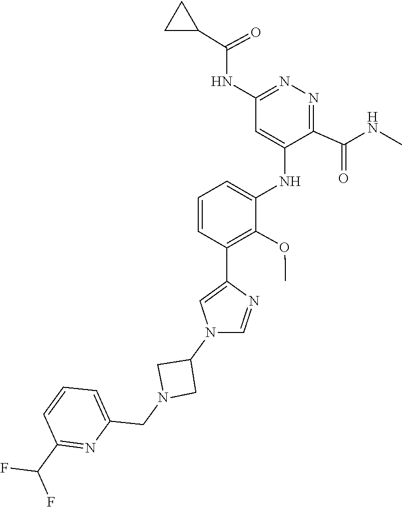 [SMILES: CNC(=O)c1nnc(NC(=O)C2CC2)cc1Nc1cccc(-c2cn(C3CN(Cc4cccc(C(F)F)n4)C3)cn2)c1OC] | A | | C | C |
| 342 | 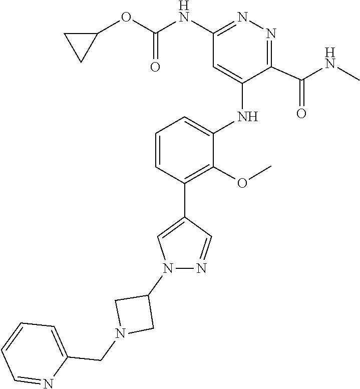 [SMILES: CNC(=O)c1nnc(NC(=O)OC2CC2)cc1Nc1cccc(-c2cnn(C3CN(Cc4ccccn4)C3)c2)c1OC] | A | | D | D |
| 343 | 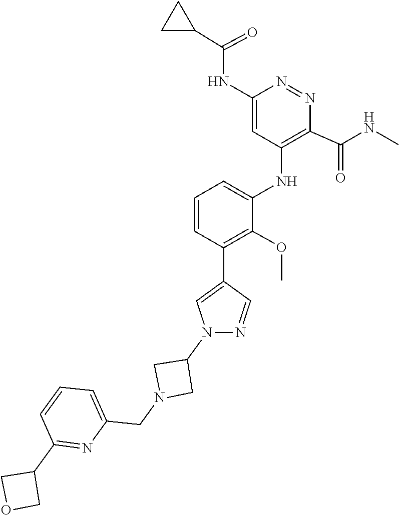 [SMILES: CNC(=O)c1nnc(NC(=O)C2CC2)cc1Nc1cccc(-c2cnn(C3CN(Cc4cccc(C5COC5)n4)C3)c2)c1OC] | A | | C | C |
| 344 | 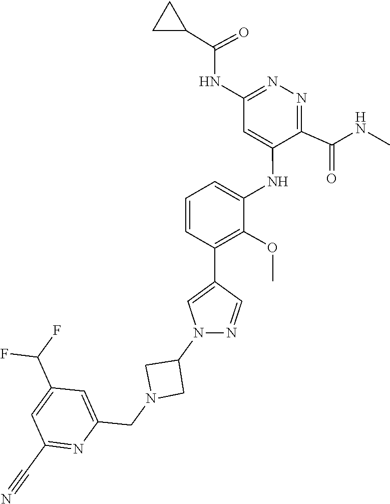 [SMILES: CNC(=O)c1nnc(NC(=O)C2CC2)cc1Nc1cccc(-c2cnn(C3CN(Cc4cc(C(F)F)cc(C#N)n4)C3)c2)c1OC] | A | | C | C |
| 345 | 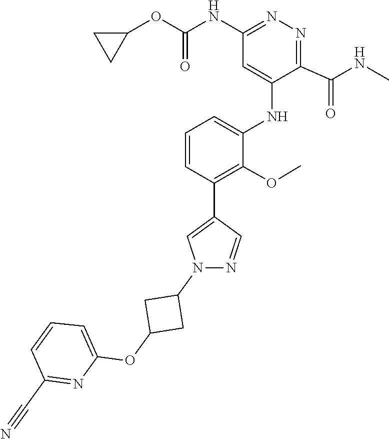 [SMILES: CNC(=O)c1nnc(NC(=O)OC2CC2)cc1Nc1cccc(-c2cnn(C3CC(Oc4cccc(C#N)n4)C3)c2)c1OC] | A | | E | D |
| 346 | 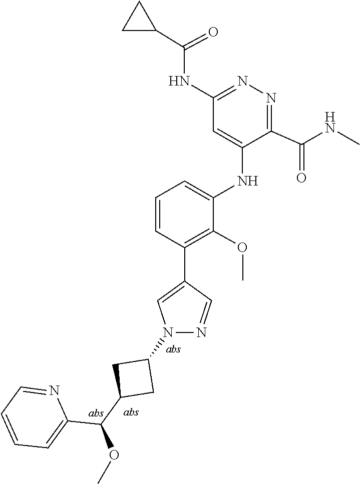 [SMILES: CNC(=O)c1nnc(NC(=O)C2CC2)cc1Nc1cccc(-c2cnn([C@H]3C[C@H]([C@@H](OC)c4ccccn4)C3)c2)c1OC] | A | | D | D |
| 347 | 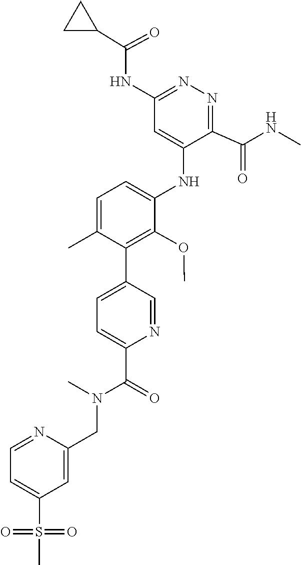 [SMILES: CNC(=O)c1nnc(NC(=O)C2CC2)cc1Nc1ccc(C)c(-c2ccc(C(=O)N(C)Cc3cc(S(C)(=O)=O)ccn3)nc2)c1OC] | A | | C | C |
| 348 | 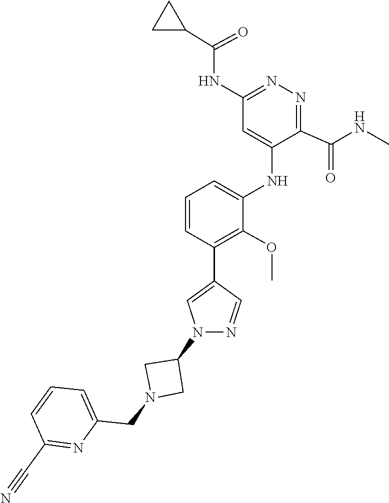 | A | | C | C |
| 349 | 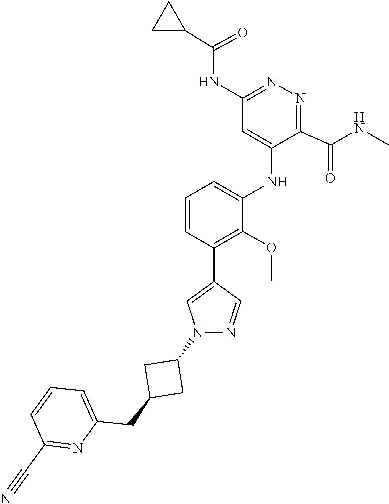 [SMILES: CNC(=O)c1nnc(NC(=O)C2CC2)cc1Nc1cccc(-c2cnn([C@H]3C[C@H](Cc4cccc(C#N)n4)C3)c2)c1OC] | A | | D | D |
| 350 | 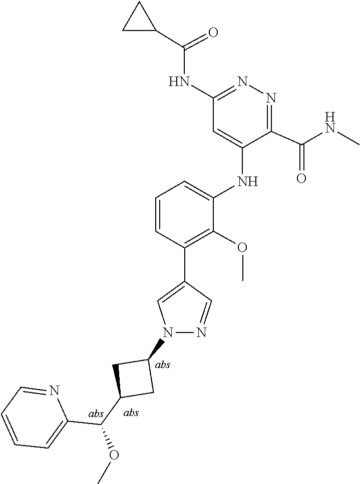 [SMILES: CNC(=O)c1nnc(NC(=O)C2CC2)cc1Nc1cccc(-c2cnn([C@H]3C[C@@H]([C@H](OC)c4ccccn4)C3)c2)c1OC] | B | | D | D |
| 351 | 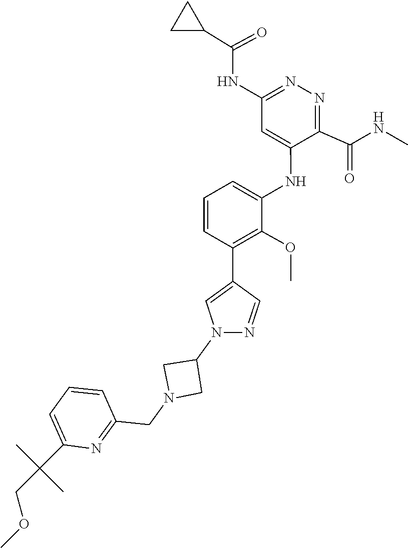 | A | | D | D |
| 352 | 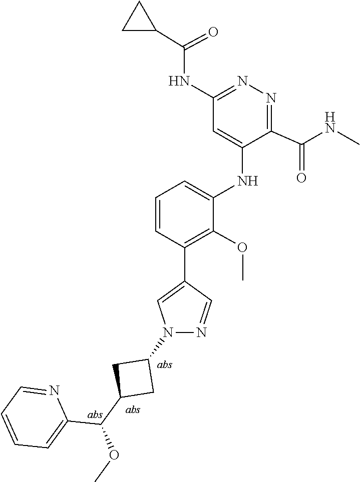 [SMILES: CNC(=O)c1nnc(NC(=O)C2CC2)cc1Nc1cccc(-c2cnn([C@H]3C[C@H]([C@H](OC)c4ccccn4)C3)c2)c1OC] | A | | E | E |
| 353 | 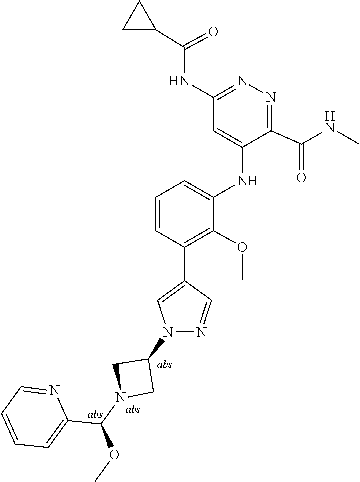 | A | | D | D |
| 354 | 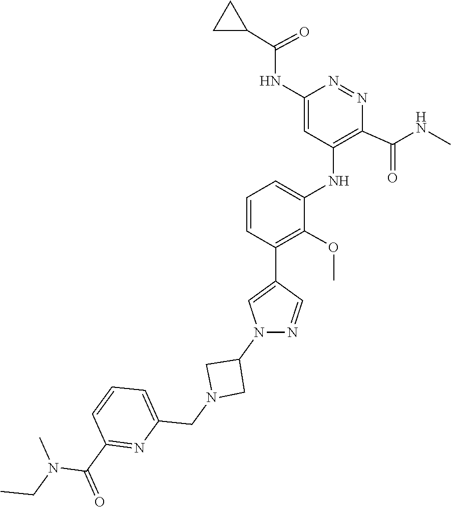 [SMILES: CCN(C)C(=O)c1cccc(CN2CC(n3cc(-c4cccc(Nc5cc(NC(=O)C6CC6)nnc5C(=O)NC)c4OC)cn3)C2)n1] | A | | C | B |
| 355 | 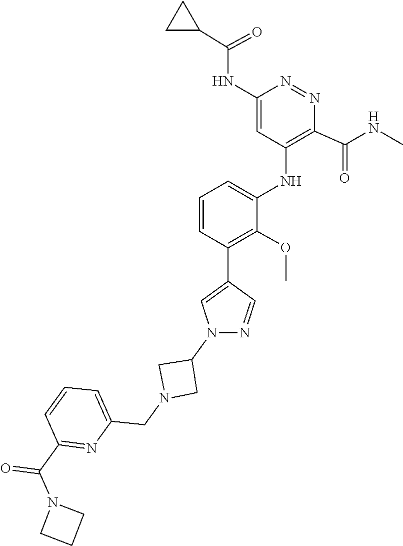 [SMILES: CNC(=O)c1nnc(NC(=O)C2CC2)cc1Nc1cccc(-c2cnn(C3CN(Cc4cccc(C(=O)N5CCC5)n4)C3)c2)c1OC] | A | | C | B |
| 356 | 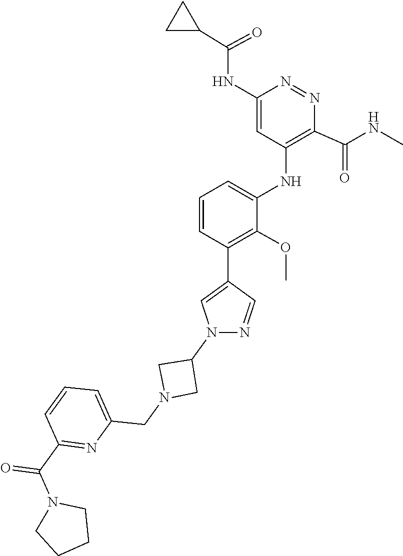 [SMILES: CNC(=O)c1nnc(NC(=O)C2CC2)cc1Nc1cccc(-c2cnn(C3CN(Cc4cccc(C(=O)N5CCCC5)n4)C3)c2)c1OC] | A | | B | B |
| 357 | 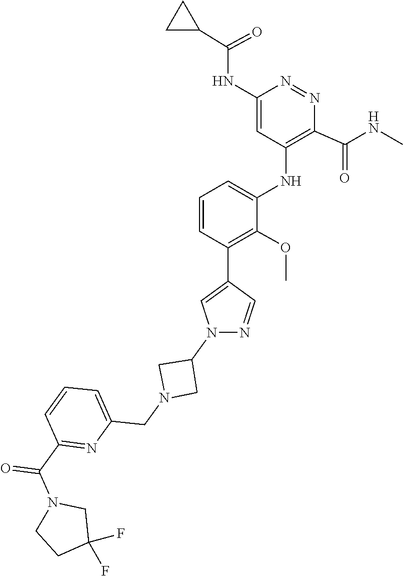 [SMILES: CNC(=O)c1nnc(NC(=O)C2CC2)cc1Nc1cccc(-c2cnn(C3CN(Cc4cccc(C(=O)N5CCC(F)(F)C5)n4)C3)c2)c1OC] | A | | C | B |
| 358 | 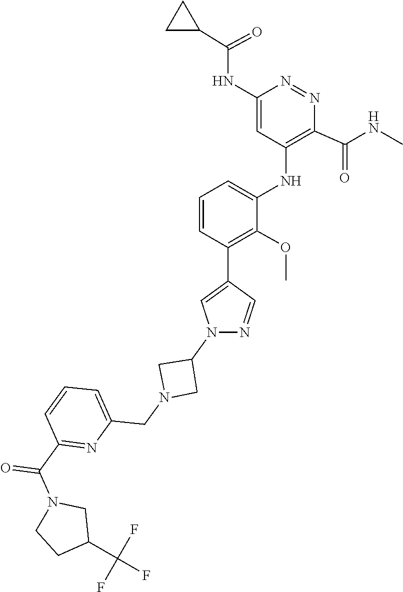 [SMILES: CNC(=O)c1nnc(NC(=O)C2CC2)cc1Nc1cccc(-c2cnn(C3CN(Cc4cccc(C(=O)N5CCC(C(F)(F)F)C5)n4)C3)c2)c1OC] | A | | C | C |
| 359 | 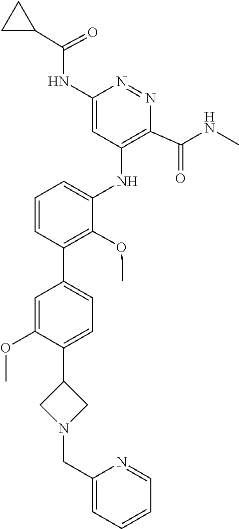 [SMILES: CNC(=O)c1nnc(NC(=O)C2CC2)cc1Nc1cccc(-c2ccc(C3CN(Cc4ccccn4)C3)c(OC)c2)c1OC] | C | | E | D |
| 360 | 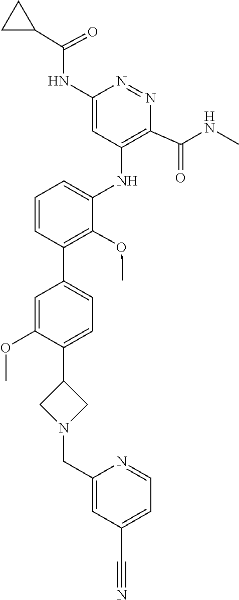 [SMILES: CNC(=O)c1nnc(NC(=O)C2CC2)cc1Nc1cccc(-c2ccc(C3CN(Cc4cc(C#N)ccn4)C3)c(OC)c2)c1OC] | C | | E | D |
| 361 | 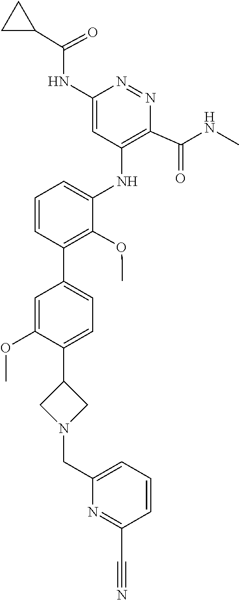 [SMILES: CNC(=O)c1nnc(NC(=O)C2CC2)cc1Nc1cccc(-c2ccc(C3CN(Cc4cccc(C#N)n4)C3)c(OC)c2)c1OC] | C | | E | D |
| 362 | 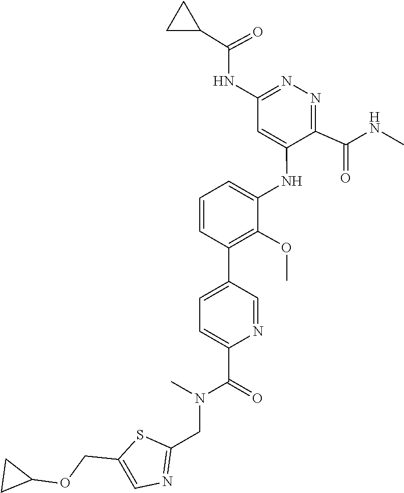 [SMILES: CNC(=O)c1nnc(NC(=O)C2CC2)cc1Nc1cccc(-c2ccc(C(=O)N(C)Cc3ncc(COC4CC4)s3)nc2)c1OC] | A | | D | D |
| 363 | 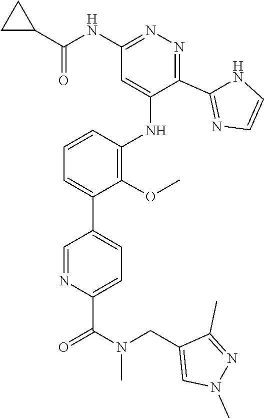 [SMILES: COc1c(Nc2cc(NC(=O)C3CC3)nnc2-c2ncc[nH]2)cccc1-c1ccc(C(=O)N(C)Cc2cn(C)nc2C)nc1] | A | | D | C |
| 364 | 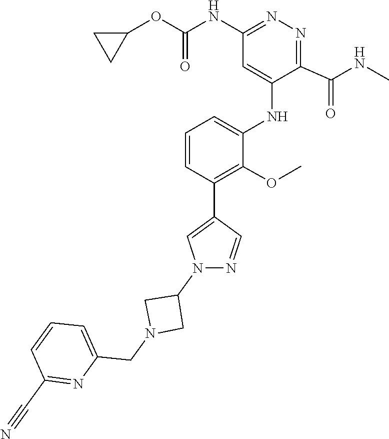 [SMILES: CNC(=O)c1nnc(NC(=O)OC2CC2)cc1Nc1cccc(-c2cnn(C3CN(Cc4cccc(C#N)n4)C3)c2)c1OC] | A | | C | C |
| 365 | 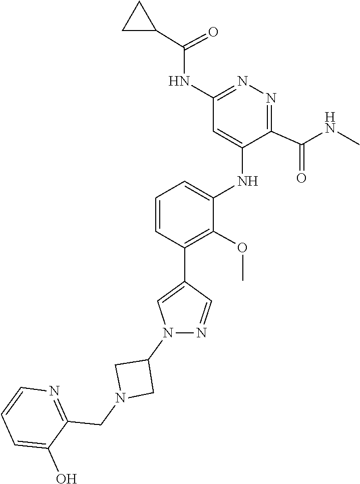 [SMILES: CNC(=O)c1nnc(NC(=O)C2CC2)cc1Nc1cccc(-c2cnn(C3CN(Cc4ncccc4O)C3)c2)c1OC] | A | | D | D |
| 366 | 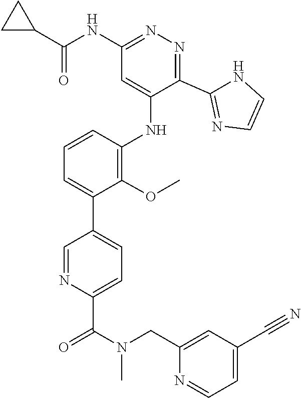 [SMILES: COc1c(Nc2cc(NC(=O)C3CC3)nnc2-c2ncc[nH]2)cccc1-c1ccc(C(=O)N(C)Cc2cc(C#N)ccn2)nc1] | A | | C | C |
| 367 | 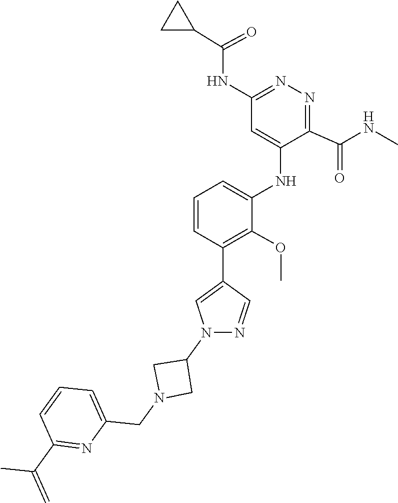 [SMILES: C=C(C)c1cccc(CN2CC(n3cc(-c4cccc(Nc5cc(NC(=O)C6CC6)nnc5C(=O)NC)c4OC)cn3)C2)n1] | A | | C | C |
| 368 | 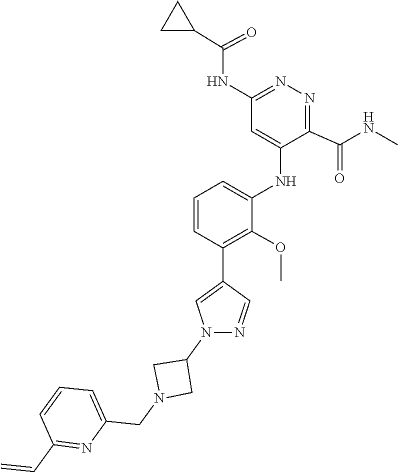 [SMILES: C=Cc1cccc(CN2CC(n3cc(-c4cccc(Nc5cc(NC(=O)C6CC6)nnc5C(=O)NC)c4OC)cn3)C2)n1] | A | | C | C |
| 369 | 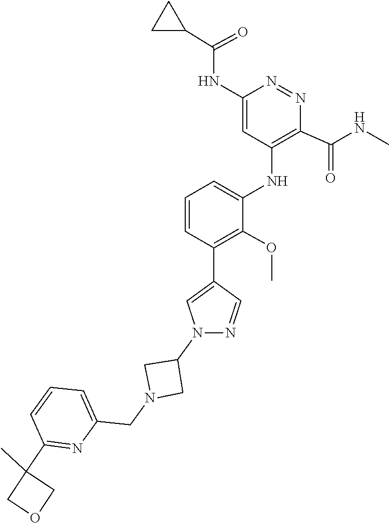 | A | | C | C |
| 370 | 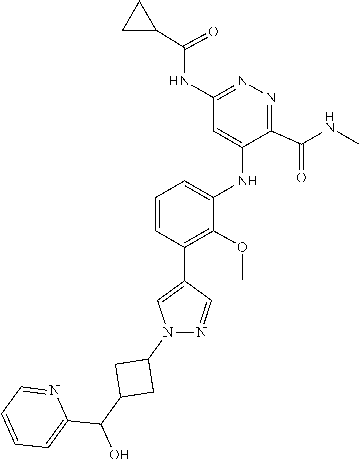 [SMILES: CNC(=O)c1nnc(NC(=O)C2CC2)cc1Nc1cccc(-c2cnn(C3CC(C(O)c4ccccn4)C3)c2)c1OC] | A | | D | D |
| 371 | 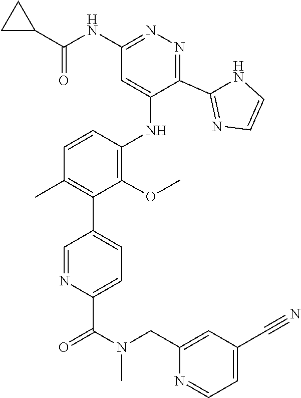 [SMILES: COc1c(Nc2cc(NC(=O)C3CC3)nnc2-c2ncc[nH]2)ccc(C)c1-c1ccc(C(=O)N(C)Cc2cc(C#N)ccn2)nc1] | A | | C | C |
| 372 | 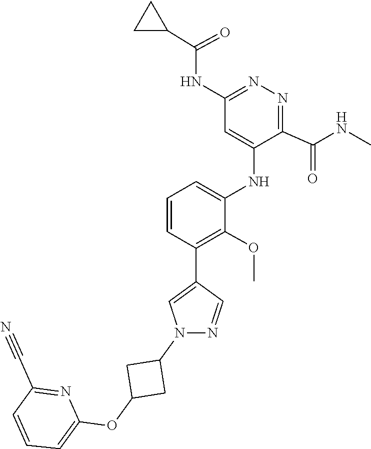 [SMILES: CNC(=O)c1nnc(NC(=O)C2CC2)cc1Nc1cccc(-c2cnn(C3CC(Oc4cccc(C#N)n4)C3)c2)c1OC] | A | | D | D |
| 373 | 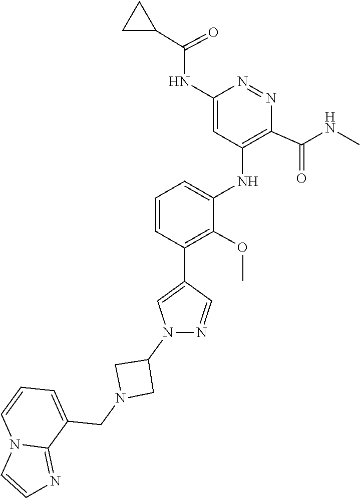 [SMILES: CNC(=O)c1nnc(NC(=O)C2CC2)cc1Nc1cccc(-c2cnn(C3CN(Cc4cccn5ccnc45)C3)c2)c1OC] | A | | C | C |
| 374 | 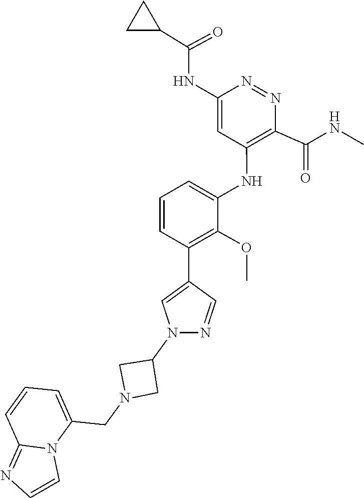 [SMILES: CNC(=O)c1nnc(NC(=O)C2CC2)cc1Nc1cccc(-c2cnn(C3CN(Cc4cccc5nccn45)C3)c2)c1OC] | A | | C | C |
| 375 | 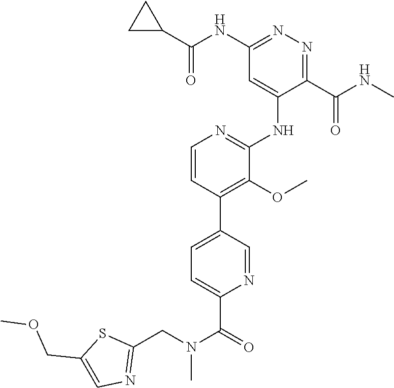 | A | | | |
| 376 | 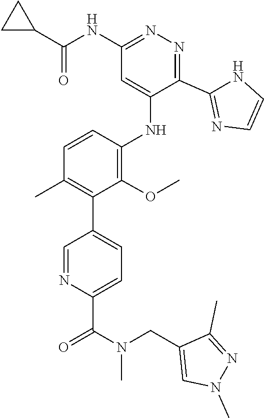 [SMILES: COc1c(Nc2cc(NC(=O)C3CC3)nnc2-c2ncc[nH]2)ccc(C)c1-c1ccc(C(=O)N(C)Cc2cn(C)nc2C)nc1] | A | | D | D |
| 377 | 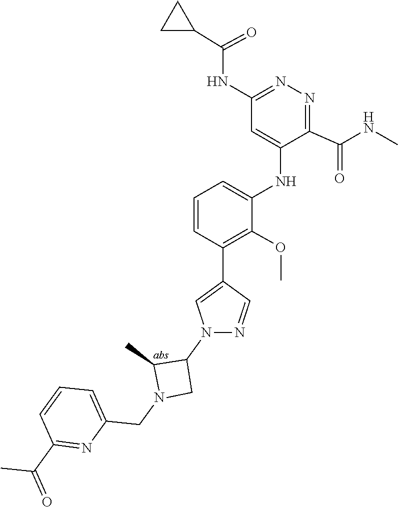 [SMILES: CNC(=O)c1nnc(NC(=O)C2CC2)cc1Nc1cccc(-c2cnn(C3CN(Cc4cccc(C(C)=O)n4)[C@H]3C)c2)c1OC] | A | | D | D |
| 378 | 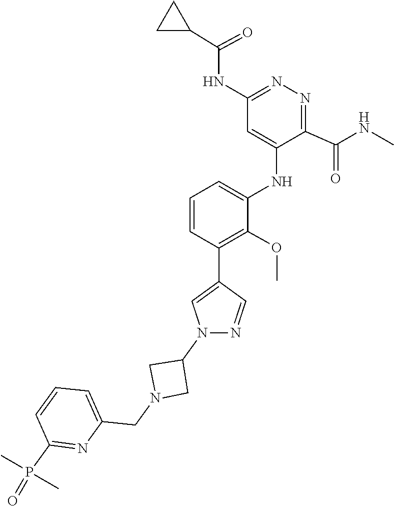 | A | | D | D |
| 379 | 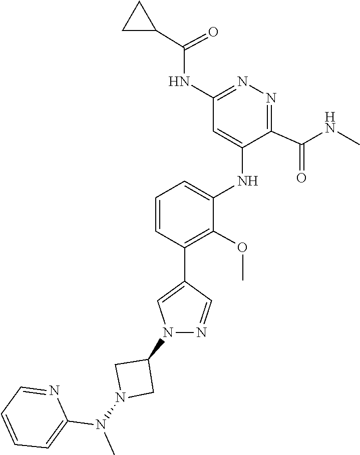 | A | | D | D |
| 380 | 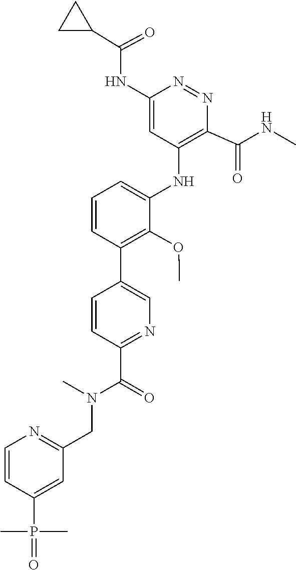 | A | | E | E |
| 381 | 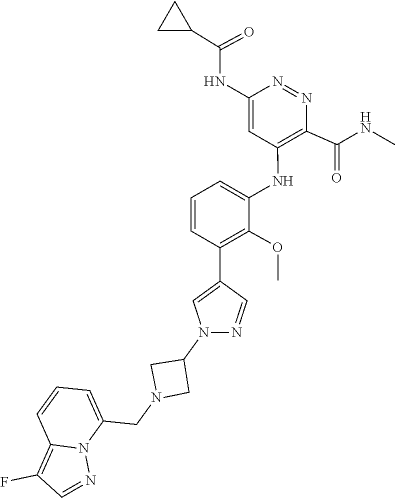 [SMILES: CNC(=O)c1nnc(NC(=O)C2CC2)cc1Nc1cccc(-c2cnn(C3CN(Cc4cccc5c(F)cnn45)C3)c2)c1OC] | A | | C | C |
| 382 | 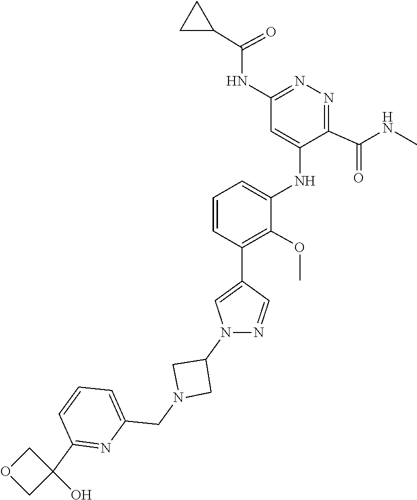 [SMILES: CNC(=O)c1nnc(NC(=O)C2CC2)cc1Nc1cccc(-c2cnn(C3CN(Cc4cccc(C5(O)COC5)n4)C3)c2)c1OC] | A | | C | C |
| 383 | 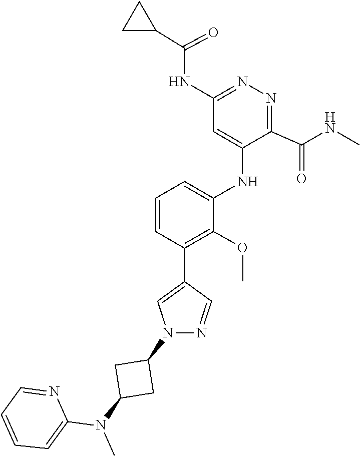 [SMILES: CNC(=O)c1nnc(NC(=O)C2CC2)cc1Nc1cccc(-c2cnn([C@H]3C[C@@H](N(C)c4ccccn4)C3)c2)c1OC] | A | | E | E |
| 384 | 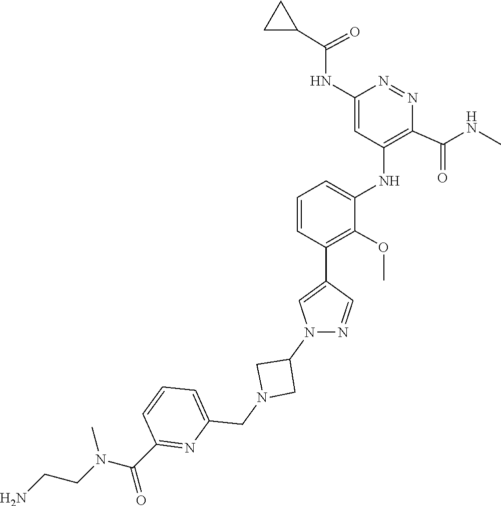 [SMILES: CNC(=O)c1nnc(NC(=O)C2CC2)cc1Nc1cccc(-c2cnn(C3CN(Cc4cccc(C(=O)N(C)CCN)n4)C3)c2)c1OC] | A | | D | D |
| 385 | 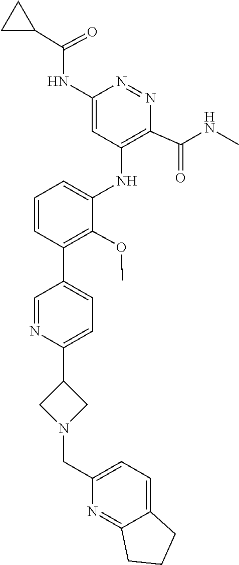 [SMILES: CNC(=O)c1nnc(NC(=O)C2CC2)cc1Nc1cccc(-c2ccc(C3CN(Cc4ccc5c(n4)CCC5)C3)nc2)c1OC] | C | | E | D |
| 386 | 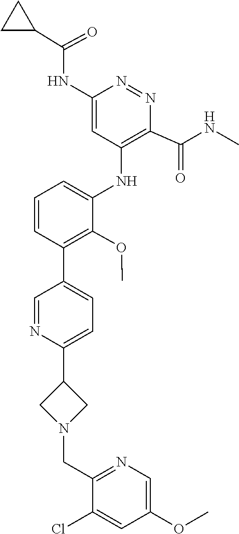 [SMILES: CNC(=O)c1nnc(NC(=O)C2CC2)cc1Nc1cccc(-c2ccc(C3CN(Cc4ncc(OC)cc4Cl)C3)nc2)c1OC] | | | E | D |
| 387 | 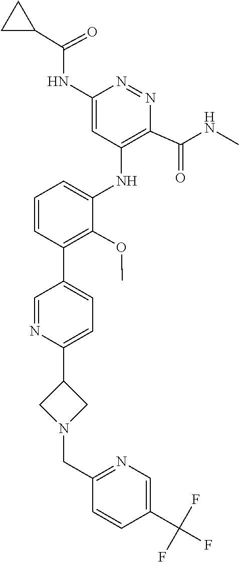 [SMILES: CNC(=O)c1nnc(NC(=O)C2CC2)cc1Nc1cccc(-c2ccc(C3CN(Cc4ccc(C(F)(F)F)cn4)C3)nc2)c1OC] | | | E | D |
| 388 | 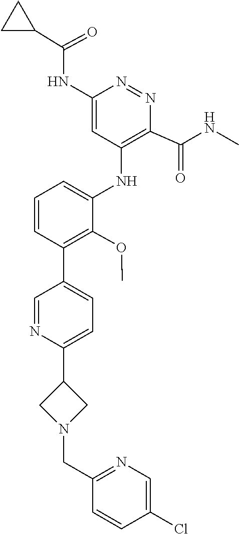 [SMILES: CNC(=O)c1nnc(NC(=O)C2CC2)cc1Nc1cccc(-c2ccc(C3CN(Cc4ccc(Cl)cn4)C3)nc2)c1OC] | C | | E | D |
| 389 | 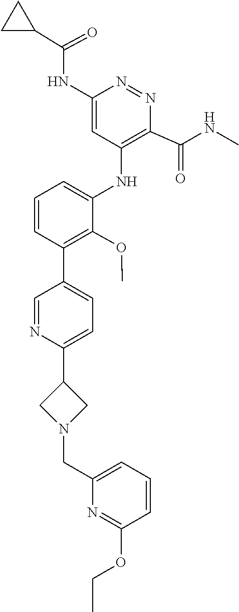 [SMILES: CCOc1cccc(CN2CC(c3ccc(-c4cccc(Nc5cc(NC(=O)C6CC6)nnc5C(=O)NC)c4OC)cn3)C2)n1] | C | | E | D |
| 390 | 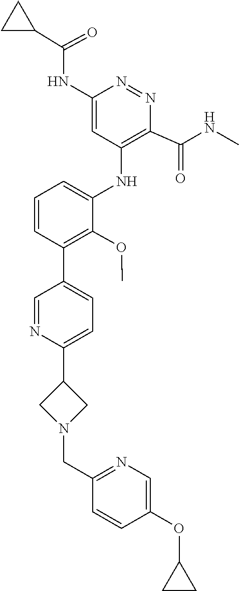 [SMILES: CNC(=O)c1nnc(NC(=O)C2CC2)cc1Nc1cccc(-c2ccc(C3CN(Cc4ccc(OC5CC5)cn4)C3)nc2)c1OC] | C | | | |
| 391 | 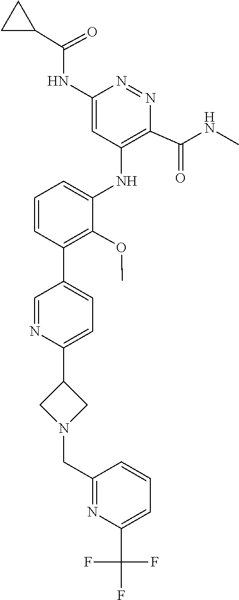 [SMILES: CNC(=O)c1nnc(NC(=O)C2CC2)cc1Nc1cccc(-c2ccc(C3CN(Cc4cccc(C(F)(F)F)n4)C3)nc2)c1OC] | B | | E | D |
| 392 | 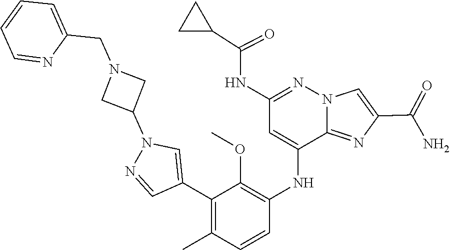 [SMILES: COc1c(Nc2cc(NC(=O)C3CC3)nn3cc(C(N)=O)nc23)ccc(C)c1-c1cnn(C2CN(Cc3ccccn3)C2)c1] | | | E | E |
| 393 | 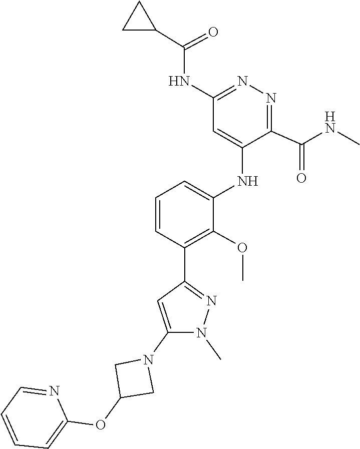 [SMILES: CNC(=O)c1nnc(NC(=O)C2CC2)cc1Nc1cccc(-c2cc(N3CC(Oc4ccccn4)C3)n(C)n2)c1OC] | | | D | E |
| 394 | 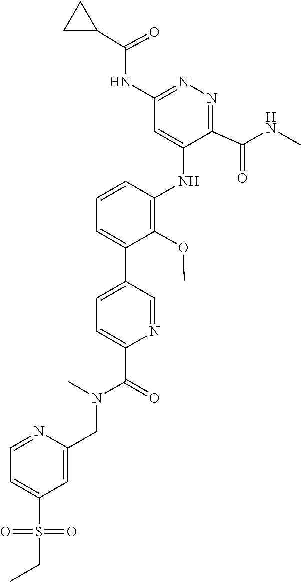 [SMILES: CCS(=O)(=O)c1ccnc(CN(C)C(=O)c2ccc(-c3cccc(Nc4cc(NC(=O)C5CC5)nnc4C(=O)NC)c3OC)cn2)c1] | A | | B | B |
| 395 | 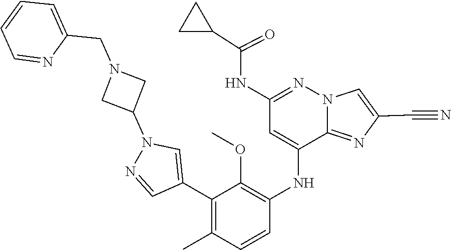 [SMILES: COc1c(Nc2cc(NC(=O)C3CC3)nn3cc(C#N)nc23)ccc(C)c1-c1cnn(C2CN(Cc3ccccn3)C2)c1] | | | E | E |
| 396 | 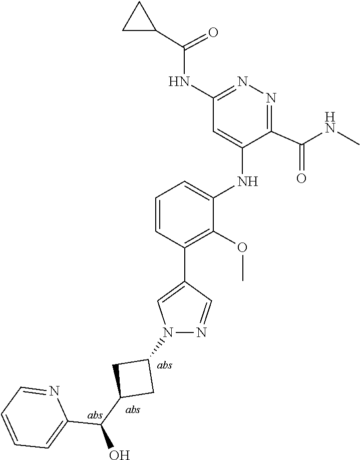 [SMILES: CNC(=O)c1nnc(NC(=O)C2CC2)cc1Nc1cccc(-c2cnn([C@H]3C[C@H]([C@@H](O)c4ccccn4)C3)c2)c1OC] | A | | D | D |
| 397 | 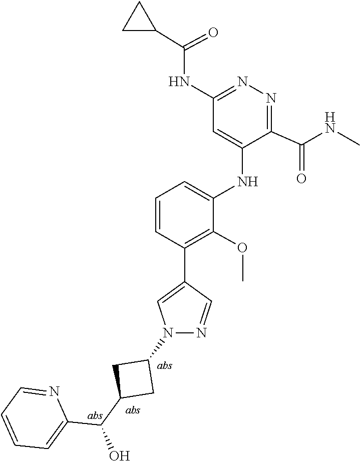 [SMILES: CNC(=O)c1nnc(NC(=O)C2CC2)cc1Nc1cccc(-c2cnn([C@H]3C[C@H]([C@H](O)c4ccccn4)C3)c2)c1OC] | A | | D | E |
| 398 | 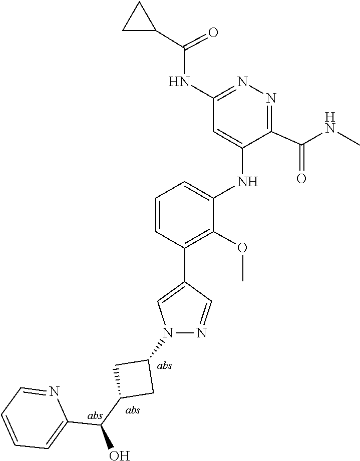 [SMILES: CNC(=O)c1nnc(NC(=O)C2CC2)cc1Nc1cccc(-c2cnn([C@H]3C[C@@H]([C@@H](O)c4ccccn4)C3)c2)c1OC] | A | | D | D |
| 399 | 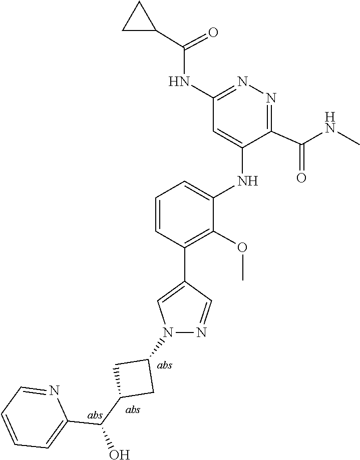 [SMILES: CNC(=O)c1nnc(NC(=O)C2CC2)cc1Nc1cccc(-c2cnn([C@H]3C[C@@H]([C@H](O)c4ccccn4)C3)c2)c1OC] | A | | D | C |
| 400 | 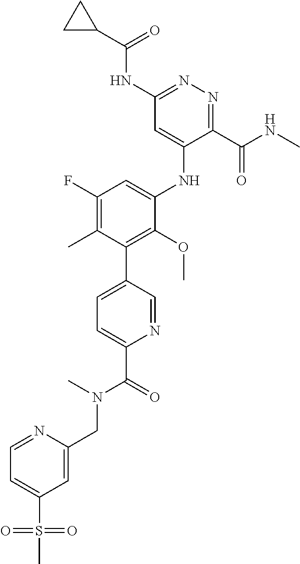 [SMILES: CNC(=O)c1nnc(NC(=O)C2CC2)cc1Nc1cc(F)c(C)c(-c2ccc(C(=O)N(C)Cc3cc(S(C)(=O)=O)ccn3)nc2)c1OC] | | | | C |
| 401 | 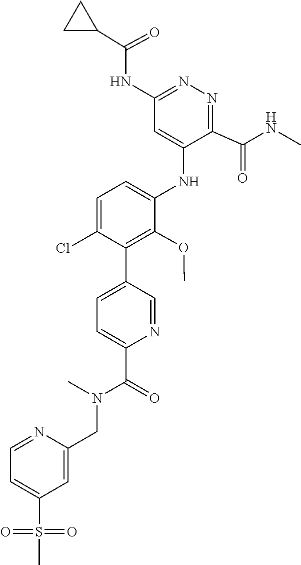 [SMILES: CNC(=O)c1nnc(NC(=O)C2CC2)cc1Nc1ccc(Cl)c(-c2ccc(C(=O)N(C)Cc3cc(S(C)(=O)=O)ccn3)nc2)c1OC] | | | | C |
| 402 | 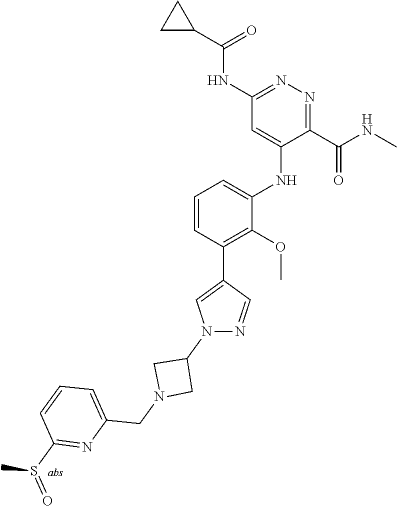 | | | | C |
| 403 | 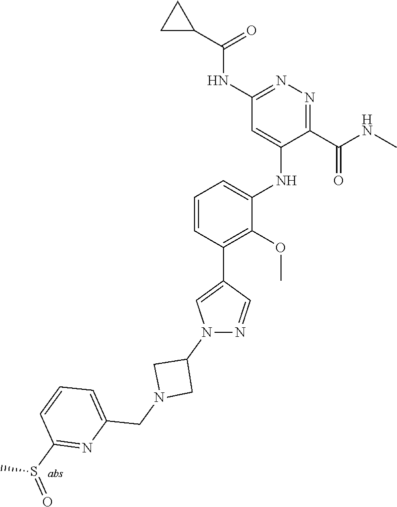 | | | | C |
| 404 | 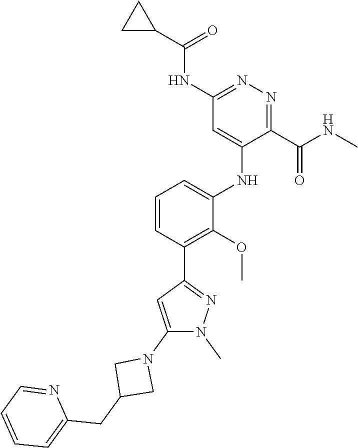 [SMILES: CNC(=O)c1nnc(NC(=O)C2CC2)cc1Nc1cccc(-c2cc(N3CC(Cc4ccccn4)C3)n(C)n2)c1OC] | | | | E |
| 405 | 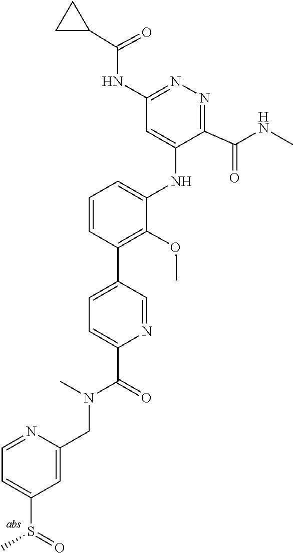 [SMILES: CNC(=O)c1nnc(NC(=O)C2CC2)cc1Nc1cccc(-c2ccc(C(=O)N(C)Cc3cc([S@@](C)=O)ccn3)nc2)c1OC] | | | | C |
| 406 | 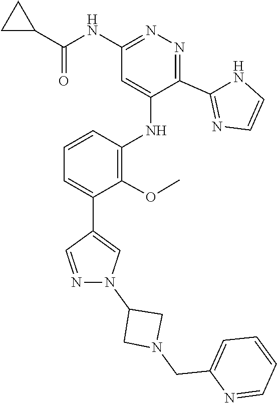 [SMILES: COc1c(Nc2cc(NC(=O)C3CC3)nnc2-c2ncc[nH]2)cccc1-c1cnn(C2CN(Cc3ccccn3)C2)c1] | | | | E |
| 407 | 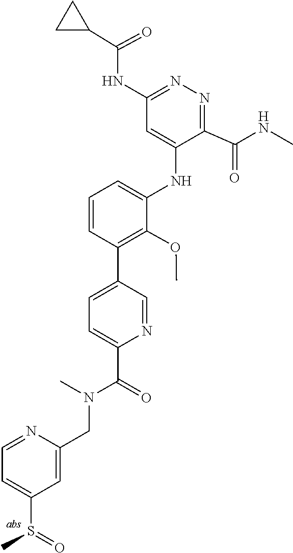 [SMILES: CNC(=O)c1nnc(NC(=O)C2CC2)cc1Nc1cccc(-c2ccc(C(=O)N(C)Cc3cc([S@](C)=O)ccn3)nc2)c1OC] | | | | D |
| 408 | 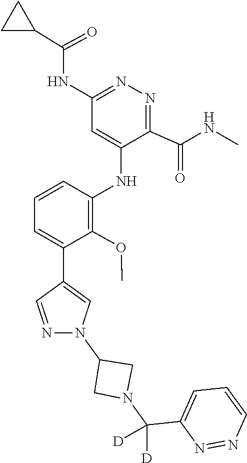 [SMILES: [2H]C([2H])(c1cccnn1)N1CC(n2cc(-c3cccc(Nc4cc(NC(=O)C5CC5)nnc4C(=O)NC)c3OC)cn2)C1] | A | | C | C |
| 409 | 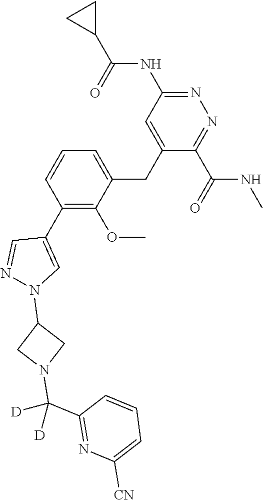 [SMILES: [2H]C([2H])(c1cccc(C#N)n1)N1CC(n2cc(-c3cccc(Cc4cc(NC(=O)C5CC5)nnc4C(=O)NC)c3OC)cn2)C1] | A | | C | C |

In chemical structures in Table 1, above, and the Examples, below, stereogenic centers are described according to the Enhanced Stereo Representation format (MDL/Biovia, e.g. using labels “or1”, “or2”, “abs”, “and1”).

In some embodiments, the present disclosure comprises a compound of formula I or I′ selected from those depicted in Table 1, above, or a pharmaceutically acceptable salt, stereoisomer, or mixture of stereoisomers thereof. In some embodiments, the present disclosure provides a compound of formula I or I′ selected from those depicted in Table 1, above, or a pharmaceutically acceptable salt thereof. In some embodiments, the present disclosure provides a compound of formula I or I′ selected from those depicted in Table 1, above.

### 4. Uses, Formulation, and Administration

### Pharmaceutically Acceptable Compositions

According to another embodiment, the disclosure provides a composition comprising a compound of this disclosure, or a pharmaceutically acceptable derivative thereof, and a pharmaceutically acceptable carrier, adjuvant, or vehicle. In some embodiments, the disclosure provides a pharmaceutical composition comprising a compound of this disclosure, and a pharmaceutically acceptable carrier. The amount of compound in compositions of this disclosure is such that is effective to measurably inhibit a JAK2 protein kinase, or a mutant thereof, in a biological sample or in a patient. In certain embodiments, the amount of compound in compositions of this disclosure is such that it is effective to measurably inhibit a JAK2 protein kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of a JAK2 protein kinase, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range, and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E, eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the inhibition of a kinase or a mutant thereof. In some embodiments, the kinase inhibited by the compounds and compositions described herein is JAK2. In some embodiments, the kinase inhibited by the compounds and compositions described herein is one or more of a JAK1, JAK2, JAK3. In some embodiments, the kinase inhibited by the compounds and compositions described herein is a JAK2 containing a V617F mutation.

Compounds or compositions of the disclosure can be useful in applications that benefit from inhibition of JAK2 enzymes. For example, JAK2 inhibitors of the present disclosure are useful for the treatment of cellular proliferative diseases generally. Compounds or compositions of the disclosure can be useful in applications that benefit from inhibition of JAK2 enzymes. For example, JAK2 inhibitors of the present disclosure are useful for the treatment of cellular proliferative diseases generally.

The activity of a compound utilized in this disclosure as an inhibitor of a JAK2 kinase, or a mutant thereof, may be assayed in vitro, in vivo or in a cell line. In vitro assays include assays that determine inhibition of either the phosphorylation activity and/or the subsequent functional consequences, of an activated JAK2 enzyme, or a mutant thereof. Alternative in vitro assays quantitate the ability of the inhibitor to bind to a JAK2 enzyme. Inhibitor binding may be measured by radiolabeling the inhibitor prior to binding, isolating the inhibitor/JAK2 complex and determining the amount of radiolabel bound. Alternatively, inhibitor binding may be determined by running a competition experiment where new inhibitors are incubated with a JAK2 bound to known radioligands. Alternatively, inhibitor binding may be determined by a biophysical method such as surface plasmon resonance (SPR). Representative in vitro and in vivo assays useful in assaying a JAK2 inhibitor include those described and disclosed in the patent and scientific publications described herein. Detailed conditions for assaying a compound utilized in this disclosure as an inhibitor of a JAK2, or a mutant thereof, are set forth in the Examples below.

### Treatment of Disorders

Provided compounds are inhibitors of JAK2 and are therefore useful for treating one or more disorders associated with activity of JAK2 or mutants thereof. Thus, in certain embodiments, the present disclosure provides a method of treating a JAK2-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating a JAK2-mediated disorder in a subject comprising administering a therapeutically effective amount of a compound of the present disclosure, or a pharmaceutically acceptable composition thereof, to a subject in need thereof. In some embodiments, the subject has a mutant JAK2. In some embodiments, the subject has JAK2 containing a V617F mutations.

As used herein, the term “JAK2-mediated” disorders, diseases, and/or conditions means any disease or other deleterious condition in which JAK2 or a mutant thereof is known to play a role. Accordingly, another embodiment of the present disclosure relates to treating or lessening the severity of one or more diseases in which JAK2, or a mutant thereof, is known to play a role. Such JAK2-mediated disorders include, but are not limited to, cellular proliferative disorders (e.g. cancer). In some embodiments, the JAK2-mediated disorder is a disorder mediated by a mutant JAK2. In some embodiments, the JAK2-mediated disorder is a disorder mediated by a JAK2 containing a V617F mutations.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing.

In some embodiments, the present disclosure provides a method for treating a cellular proliferative disease, said method comprising administering to a patient in need thereof, a therapeutically effective amount of a compound of the present disclosure, or a pharmaceutically acceptable composition thereof.

In some embodiments, the method of treatment comprises the steps of: i) identifying a subject in need of such treatment; (ii) providing a disclosed compound, or a pharmaceutically acceptable salt thereof; and (iii) administering said provided compound in a therapeutically effective amount to treat, suppress and/or prevent the disease state or condition in a subject in need of such treatment. In some embodiments, the subject has a mutant JAK2. In some embodiments, the subject has JAK2 containing a V617F mutation.

In some embodiments, the method of treatment comprises the steps of: i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment. In some embodiments, the subject has a mutant JAK2. In some embodiments, the subject has JAK2 containing a V617F mutation.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a hematopoietic cancer. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; acute megakaryocytic leukemia; chronic myelogenous leukemia; lymphocytic leukemia; myeloid leukemia; T-cell acute lymphoblastic leukemia (T-ALL); B-cell acute lymphoblastic leukemia (B-ALL); acute myeloid leukemia (AML); Chronic Myelomonocytic Leukemia (CMML); T-cell large granular lymphocytic leukemia (T-LGL); T-cell prolymphocytic leukemia (T-PLL); brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cellular proliferative disease has mutant JAK2. In some embodiments, the cellular proliferative disease is a myeloproliferative disorder. In some embodiments, the cancer has mutant JAK2. In some embodiments, the hematopoietic cancer has mutant JAK2. In some embodiments, the myeloproliferative disorder has mutant JAK2.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma. In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of JAK2, or somatic mutation of JAK2.

### Additional Disorders

In some embodiments, the JAK2-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the JAK2-mediated disorder is polycythemia vera, essential thrombocythemia, or myelofibrosis with myeloid metaplasia. In some embodiments, the JAK2-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the JAK2-mediated disorder is polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia areata, erythema multiforme, dermatitis herpetiformis, or scleroderma. In some embodiments, the JAK2-mediated disorder is vitiligo, hypersensitivity angiitis, urticaria, bullous pemphigoid, lupus erythematosus, pemphisus, epidermolysis bullosa acquisita, or autoimmune haematogical disorders (e.g. haemolytic anaemia, aplastic anaemia, pure red cell anaemia and idiopathic thrombocytopenia). In some embodiments, the JAK2-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g. ulcerative colitis and Crohn's disease).

In some embodiments, the JAK2-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the JAK2-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the JAK2-mediated disorder is retinopathy, such as diabetic retinopathy or hyperbaric oxygen-induced retinopathy, and conditions characterized by elevated intraocular pressure or secretion of ocular aqueous humor, such as glaucoma.

In some embodiments, the JAK2-mediated disorder is myelofibrosis (MF), polycythemia Vera (PV), essential thrombocythemia (ET), acute megakaryocytic leukemia, T-cell acute lymphoblastic leukemia (T-ALL), B-cell acute lymphoblastic leukemia (B-ALL), acute myeloid leukemia (AML), Chronic Myelomonocytic Leukemia (CMML), T-cell large granular lymphocytic leukemia (T-LGL), T-cell prolymphocytic leukemia (T-PLL), or graft versus host disease (GVHD).

### Routes of Administration and Dosage Forms

The compounds and compositions, according to the methods of the present disclosure, may be administered using any amount and any route of administration effective for treating or lessening the severity of the disorder (e.g. a proliferative disorder). The exact amount required will vary from subject to subject, depending on the species, age, and general condition of the subject, the severity of the infection, the particular agent, its mode of administration, and the like. Compounds of the disclosure are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the disclosure relates to a method of inhibiting protein kinase activity in a biological sample comprising the step of contacting said biological sample with a compound of this disclosure, or a composition comprising said compound. According to another embodiment, the disclosure relates to a method of inhibiting activity of a JAK2, or a mutant thereof, in a biological sample comprising the step of contacting said biological sample with a compound of this disclosure, or a composition comprising said compound. According to another embodiment, the disclosure relates to a method of inhibiting activity of JAK2, or a mutant thereof, in a biological sample comprising the step of contacting said biological sample with a compound of this disclosure, or a composition comprising said compound. In some embodiments, the JAK2 is a mutant JAK2. In some embodiments, the JAK2 contains a V617F mutation.

Without wishing to be bound by any particular theory, it is contemplated that compounds of the present invention block the activity of JAK2 in a manner that allows the JAK2 enzyme to continue to signal upon activation by cytokines (e.g. EPO), while preventing the constitutive signaling of a mutant JAK2 enzyme in the absence of cytokine. Thus in the native cellular environment, where cytokines are present, the aberrant signaling of the mutant is suppressed, but cytokine-dependent signaling through JAK2 can still occur. In some embodiments, the disclosure provides a method of selectively inhibiting JAK2 over other JAK family members (including JAK1, JAK3, and TYK2). In some embodiments, the disclosure provides a method of selectively inhibiting aberrant mutant JAK2 signaling but sparing cytokine-mediated (e.g. EPO-mediated) signaling. In some embodiments, the disclosure provides a method of selectively inhibiting V617F mutant JAK2 signaling, but sparing cytokine-mediated signaling.

In some embodiments, the disclosure provides a method of selectively inhibiting a mutant JAK2 over a wild-type JAK2. In some embodiments, the disclosure provides a method of inhibiting a mutant JAK2 over wild-type JAK2 in the presence of cytokine.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a JAK2 (for example, JAK2, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound of the present disclosure, or a composition comprising said compound.

According to another embodiment, the disclosure relates to a method of inhibiting activity of a JAK2, or a mutant thereof, in a patient comprising the step of administering to said patient a compound of the present disclosure, or a composition comprising said compound. In some embodiments, the disclosure relates to a method of inhibiting activity of JAK2, or a mutant thereof, in a patient comprising the step of administering to said patient a compound of the present disclosure, or a composition comprising said compound. In some embodiments, the JAK2 is a mutant JAK2. In some embodiments, the JAK2 contains a V617F mutation.

According to another embodiment, the present disclosure provides a method for treating a disorder mediated by a JAK2, or a mutant thereof, in a patient in need thereof, comprising the step of administering to said patient a compound according to the present disclosure or pharmaceutically acceptable composition thereof. In some embodiments, the present disclosure provides a method for treating a disorder mediated by JAK2, or a mutant thereof, in a patient in need thereof, comprising the step of administering to said patient a compound according to the present disclosure or pharmaceutically acceptable composition thereof. In some embodiments, the JAK2 is a mutant JAK2. In some embodiments, the JAK2 contains aV617F mutation.

According to another embodiment, the present disclosure provides a method of inhibiting signaling activity of JAK2, or a mutant thereof, in a subject, comprising administering a therapeutically effective amount of a compound according to the present disclosure, or a pharmaceutically acceptable composition thereof, to a subject in need thereof. In some embodiments, the present disclosure provides a method of inhibiting JAK2 signaling activity in a subject, comprising administering a therapeutically effective amount of a compound according to the present disclosure, or a pharmaceutically acceptable composition thereof, to a subject in need thereof. In some embodiments, the JAK2 is a mutant JAK2. In some embodiments, the JAK2 contains a V617F mutation. In some embodiments, the subject has a V617F mutant JAK2. In some embodiments, the subject has JAK2 containing aV617F mutation.

The compounds described herein can also inhibit JAK2 function through incorporation into agents that catalyze the destruction of JAK2. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of JAK2 to the E3 ligase will thus result in the destruction of the JAK2 protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

Accordingly, in certain embodiments, the method of treatment comprises administering the compound or composition of the disclosure in combination with one or more additional therapeutic agents. In certain other embodiments, the methods of treatment comprise administering the compound or composition of the disclosure as the only therapeutic agent.

The structure of the active compounds identified by code numbers, generic or trade names may be taken from the actual edition of the standard compendium “The Merck Index” or from databases, e.g. Patents International (e.g. IMS World Publications).

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Examples of agents the combinations of this invention may also be combined with include, without limitation: an interferon (e.g. Ropeginterferon alfa-2b), see, for example Sørensen et al. Haematologica 2020, Vol. 105 (9): 2262-2272; a Bcl-2 inhibitor (e.g. Navitoclax), see Harrison et al. Journal of Clinical Oncology 40, no. 15 (May 20, 2022) 1671-1680; a bromodomain and extra-terminal domain (BET) inhibitor (e.g. Pelabresib), see Mascarenhas et al. BET inhibitor pelabresib (CPI-0610) combined with ruxolitinib in patients with myelofibrosis-JAK inhibitor-naïve or with suboptimal response to ruxolitinib-preliminary data from the MANIFEST study. Presented at: 2022 EHA Congress; Jun. 9-12, 2022; Vienna, Austria. Abstract S198; a SMAD signaling modulator (e.g. Luspatercept), see Gerds et al. Blood (2019); 134 (Supplement_1): 557; a phosphatidylinositol 3-kinase delta (PI3Kδ) inhibitor (e.g. Parsaclisib), see Yacoub et al. Addition of parsaclisib (INCB050465), a PI3Kδ inhibitor, in patients with suboptimal response to ruxolitinib: a phase 2 study in patients with myelofibrosis. Presented: 2021 AACR Annual Meeting; Apr. 9-14, 2021; week 1; virtual. Abstract: CT162; erythropoiesis-stimulating agents (e.g. Aranesp), see McMullin et al. The use of erythropoiesis-stimulating agents with ruxolitinib in patients with myelofibrosis in COMFORT-II: an open-label, phase 3 study assessing efficacy and safety of ruxolitinib versus best available therapy in the treatment of myelofibrosis. Exp Hematol Oncol. 2015 Sep. 15; 4:26; a telomerase inhibitor (e.g. Imetelstat), see Hu et al. Combination Treatment with Imetelstat, a Telomerase Inhibitor, and Ruxolitinib Depletes Myelofibrosis Hematopoietic Stem Cells and Progenitor Cells. Blood 2019; 134 (Supplement_1): 2963; an MDM2 inhibitor (e.g. Navtemadlin/KRT-232), see ClinicalTrials.gov Identifier: NCT04485260; and agents that prolong or improve pharmacokinetics such as cytochrome P450 inhibitors (i.e., inhibitors of metabolic breakdown) and CYP3 A4 inhibitors (e.g., ketokenozole and ritonavir), see Umehara et al. Drug-drug interaction (DDI) assessments of ruxolitinib, a dual substrate of CYP3A4 and CYP2C9, using a verified physiologically based pharmacokinetic (PBPK) model to support regulatory submissions. Drug Metab Pers Ther. 2019 May 30; 34 (2).

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to other classes and subclasses and species of each of these compounds, as described herein. Additional compounds of the disclosure were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### LIST OF ABBREVIATIONS

aq: aqueous
Ac: acetyl
ACN or MeCN: acetonitrile
AmF: ammonium formate
anhyd.: anhydrous
BINAP: (+)-2,2′-Bis(diphenylphosphino)-1,1′-binaphthalene
Bn: Benzyl
Boc: tert-Butyloxycarbonyl
B₂Pin₂: Bis(pinacolato)diboron
Brettphos Pd G3: [(2-Di-cyclohexylphosphino-3,6-dimethoxy-2′,4′,6′-triisopropyl-1,1′-biphenyl)-2-(2′-amino-1,1′-biphenyl)] palladium (II) methanesulfonate methanesulfonate
Bu: butyl
conc.: concentrated
CuTC: Copper (I) thiophene-2-carboxylate
dba: Dibenzylideneacetone
DAST: Diethylaminosulfur trifluoride
DBU: 1,8-Diazabicyclo[5.4.0] undec-7-ene
DCE: Dichloroethane
DCM: Dichloromethane
DIBAL-H: Diisobutylaluminium hydride
DIEA: Diisopropylamine
DMA: Dimethylacetamide
DMF: N,N-dimethylformamide
DMP: Dess-Martin periodinane
DMPU: N,N′-Dimethylpropyleneurea
DMSO: dimethylsulfoxide
DPPF: 1,1′-Bis(diphenylphosphino) ferrocene
Dtbpf: 1,1′-Bis(di-tert-butylphosphino) ferrocene
EA or EtOAc: ethyl acetate
EDCI, EDC, or EDAC: 1-Ethyl-3-(3-dimethylaminopropyl) carbodiimide
equiv or eq: molar equivalents
EPhos Pd G4: Palladium, [dicyclohexyl[3-(1-methylethoxy)-2′,4′,6′-tris(1-methylethyl) [1,1′-biphenyl]-2-yl] phosphine-κP](methanesulfonato-κO) [2′-(methylamino-κN) [1,1′-biphenyl]-2-yl-κC]-(ACI)
Et: ethyl
HATU: 1-[Bis(dimethylamino)methylene]-1H-1,2,3-triazolo[4,5-b]pyridinium 3-oxid Hexafluorophosphate
HPLC: high pressure liquid chromatography
IBX: 2-Iodoxybenzoic acid
JohnPhos: 2-[Di(tert-butyl)phosphino]-1,1′-binaphthyl
LCMS or LC-MS: liquid chromatography-mass spectrometry
LDA: Lithium diisopropylamide
LiHMDS: Lithium bis(trimethylsilyl)amide
Ms: methanesulfonyl
MOM: methoxymethyl
NBS: N-bromosuccinimide
NMR: nuclear magnetic resonance
PE: petroleum ether
PMB: p-methoxybenzyl
rt or RT: room temperature
RuPhos Pd G4: Methanesulfonato (2-dicyclohexylphosphino-2′,6′-di-i-propoxy-1,1′-biphenyl) (2′-methylamino-1,1′-biphenyl-2-yl) palladium (II)
sat: saturated
SFC: Supercritical fluid chromatography
TBS: tert-butyldimethylsilyl
TEA: triethylamine
Tf: trifluoromethanesulfonate
TFA: trifluoroacetic acid
TFAA: Trifluoroacetic anhydride
THF: tetrahydrofuran
TLC: thin layer chromatography
TMEDA: Tetramethylethylenediamine
Tol: toluene
UV: ultraviolet
Xantphos: 4,5-Bis(diphenylphosphino)-9,9-dimethylxanthene
XPhos: Dicyclohexyl[2′,4′,6′-tris(propan-2-yl) [1,1′-biphenyl]-2-yl] phosphane
XPhos Pd G3: (2-Dicyclohexylphosphino-2′,4′,6′-triisopropyl-1,1′-biphenyl) [2-(2′-amino-1,1′-biphenyl)] palladium (II) methanesulfonate
XPhos Pd G4 (XPhos Pd Gen IV): (s)-(dicyclohexyl(2′,4′,6′-triisopropyl-[1,1′-biphenyl]-2-yl)-15-phosphaneyl)(2′-(methylamino)-[1,1′-biphenyl]-2-yl) palladium (III) methanesulfonate

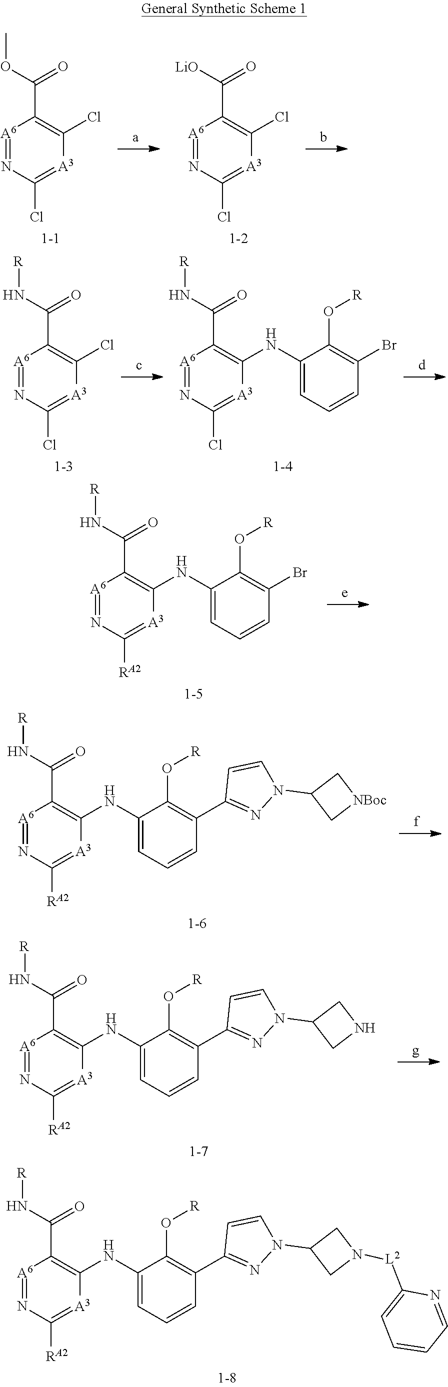

Reagents and conditions: (a) LiBr, DIEA, MeCN, H₂O, 10-25° C., 3 h; (b) MeNH₂·HCl, POCl₃, Pyridine, CH₂Cl₂, 0-20° C., 3 h; (c) 3-Bromo-2-methoxyaniline, LiHMDS, THF, −10-20° C., 1 h; (d) cyclopropanecarboxamide, K₃PO₄, Pd₂(dba)₃, DPPF, 1,4-dioxane, 25-100° C., 16 h; (e) tert-butyl 3-(4-(4,4,5,5- tetramethyl-1,3,2-dioxaborolan-2-yl)-1H-pyrazol-1-yl) azetidine-1-carboxylate, XPhos Pd Gen IV, K₃PO₄, DMF, H₂O, 90° C., 90 min; (f) TFA, CH₂Cl₂ rt, 1 h; (g) picolinaldehyde, Na(OAC)₃, Et₃N, rt, 18 h

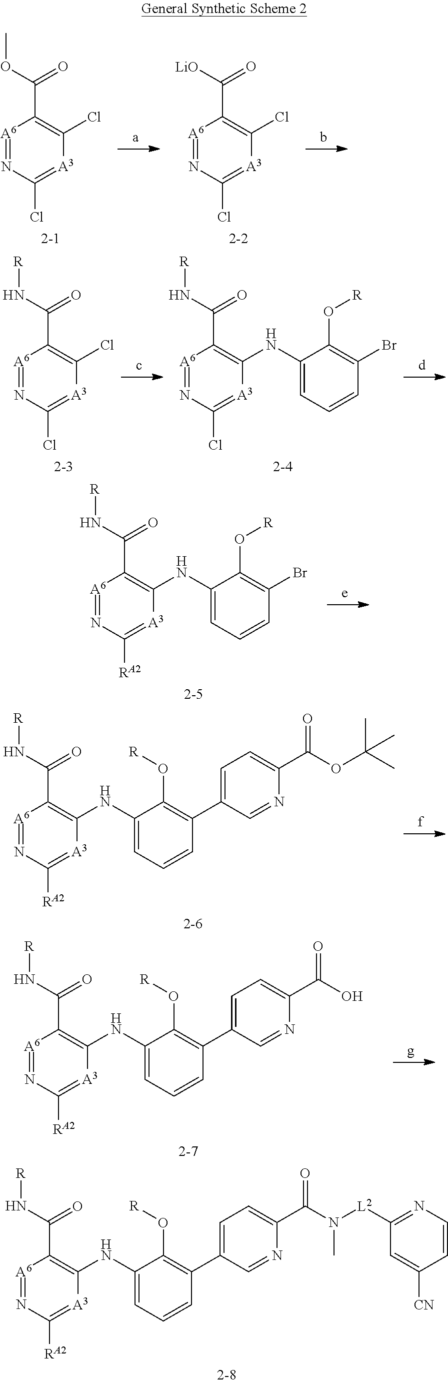

Reagents and conditions: (a) LiBr, DIEA, MeCN, H₂O, 10-25° C., 3 h; (b) MeNH₂·HCl, POCl₃, Pyridine, CH₂Cl₂, 0-20° C., 3 h; (c) 3-Bromo-2-methoxyaniline, LiHMDS, THF, −10-20° C., 1 h; (d) cyclopropanecarboxamide, K₃PO₄, Pd₂(dba)₃, DPPF, 1,4-dioxane, 25-100° C., 16 h; (e) tert-butyl 5-(4,4,5,5-tetramethyl-1,3,2-dioxaborolan-2-yl) picolinate, Pd(dppf)Cl₂·CH₂Cl₂, Cs₂CO₃, 1,4-dioxane, H₂O, 100° C., 12 h; (f) HCl in 1,4-dioxane, CH₂Cl₂ 25° C., 12 h; (g) 2-((methylamino)methyl)isonicotinonitrile, HATU, DIEA, DMF, 0 to 25° C., 2 h

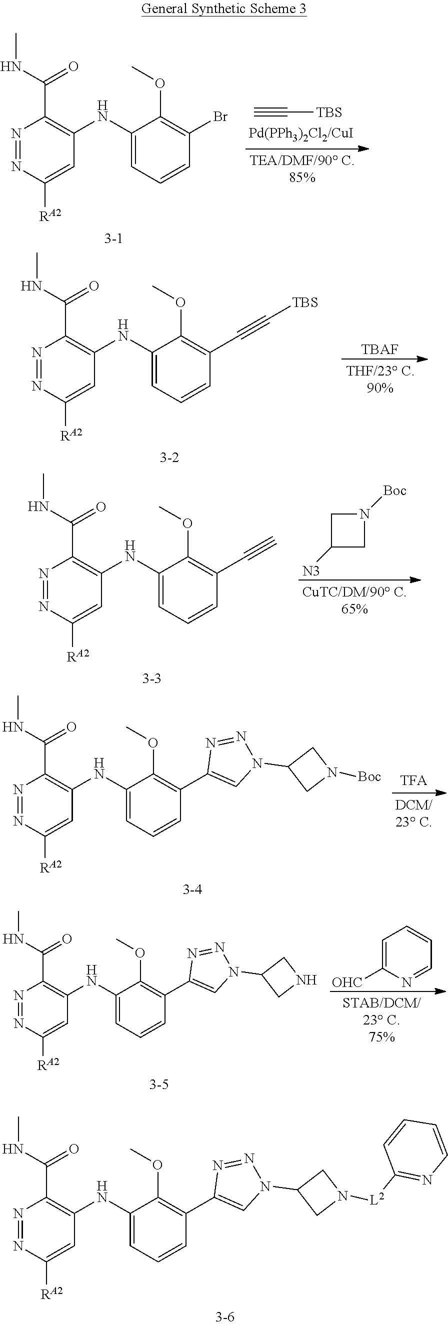

### General Procedures

The following intermediates were prepared according to the following procedures:

### Preparation of Intermediate A

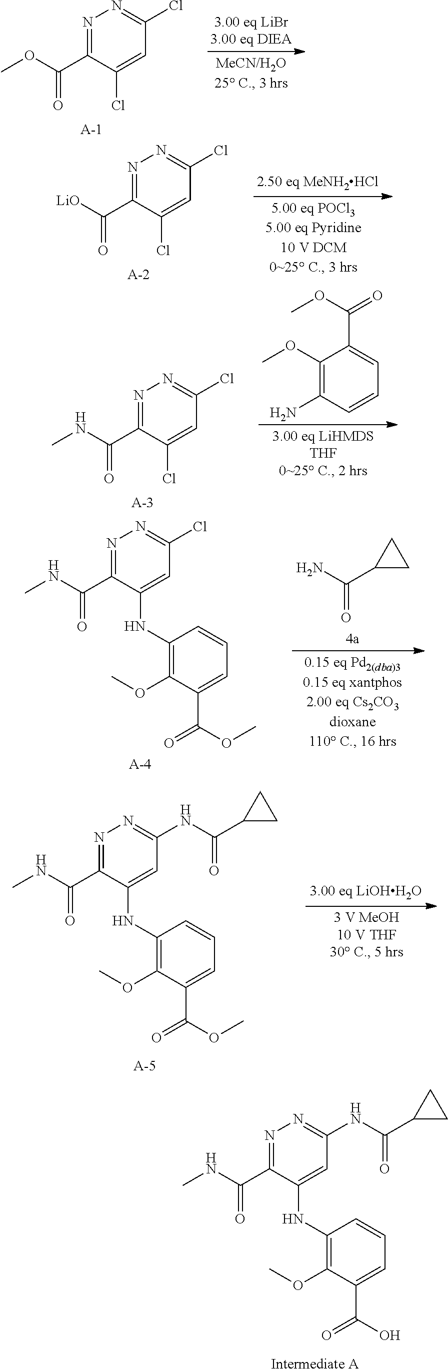

### Preparation of Compound A-2

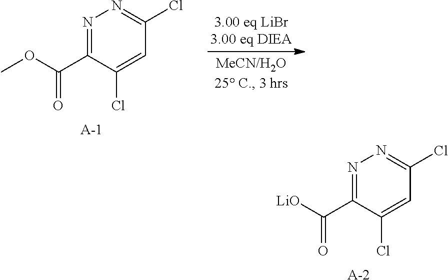

To a solution of compound A-1 (100 g, 483 mmol, 1.00 eq) in acetonitrile (500 mL) and water (75.0 mL) was added LiBr (125 g, 1.45 mol, 36.3 mL, 3.00 eq) and DIEA (187 g, 1.45 mol, 252 mL, 3.00 eq), the mixture was stirred at 25° C. for 3 hrs until the reaction was determined to be complete by LC/MS analysis. The reaction mixture was filtered and the filter cake was washed with acetronitrile (500 mL), concentrated in vacuum to give a white solid. The crude product was used to the next step. Compound A-2 (92.0 g, 462 mmol) was obtained as a white solid in 96% yield.

¹H NMR: (400 MHz, DMSO-d6): δ 8.11 (s, 1H)

### Preparation of Compound A-3

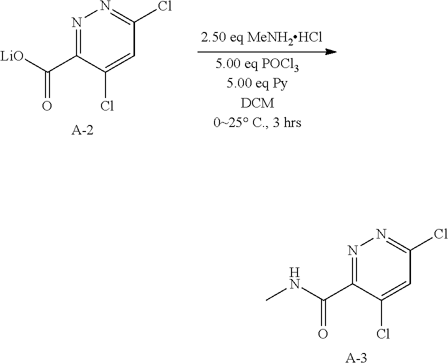

To a solution of compound A-2 (60.0 g, 301 mmol, 1.00 eq) and MeNH₂·HCl (50.9 g, 754 mmol, 2.50 eq) in DCM (600 mL) was added pyridine (119 g, 1.51 mol, 121 mL, 5.00 eq), followed by drop-wise addition of POCl₃ (231 g, 1.51 mol, 140 mL, 5.00 eq) at 0° C. The mixture was stirred at 25° C. for 3 hrs after which the reaction was determined to be complete by LC/MS analysis. The reaction mixture was then poured into H₂O (2.00 L), and the aqueous phase was extracted with dichloromethane (1.00 L×2). The combined organic phase was washed with saturated NaHCO₃ solution (2.00 L×2) and brine (2.00 L×2), dried with anhydrous Na₂SO₄, filtered and concentrated in vacuum to give a crude product. The residue was purified by trituration with petroleum ether:ethyl acetate (10:1 v/v, 50 mL) at 20° C. for 30 mins, filtered to give a yellow solid. Compound A-3 (46.0 g, 223 mmol, 74.0% yield) was obtained as a yellow solid which was confirmed by ¹H NMR.

LCMS: m/z=206.0 (M+H)+

¹H NMR: EW26719-3-P1A (400 MHZ, DMSO-d6): δ 8.93 (s, 1H), 8.47 (s, 1H), 2.83 (d, J=4.8 Hz, 3H).

### General Procedure for Preparation of Compound A-4

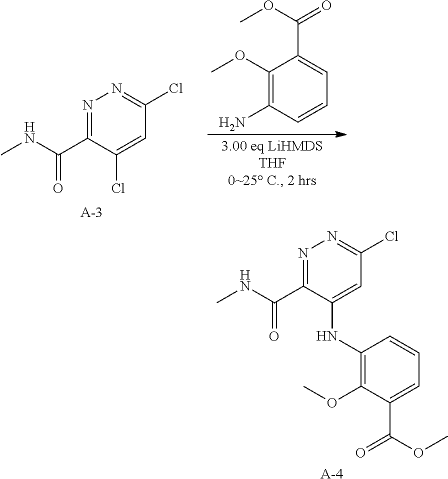

To a solution of compound A-3 (20.0 g, 97.0 mmol, 1.10 equiv) and methyl 3-amino-2-methoxybenzoate (15.9 g, 88.2 mmol, 1.00 equiv) in THF (400 mL) was added LiHMDS (1 M, 264 mL, 3.00 equiv) at 0° C. under N₂, and the mixture was stirred at 25° C. for 2 hrs until the reaction was determined to be complete by LC/MS analysis. The reaction mixture was poured into ice-cold saturated NH₄Cl solution (1.00 L), the aqueous phase was extracted with ethyl acetate (1.00 L×2). The combined organic phase was washed with brine (1.00 L×3), dried with anhydrous Na₂SO₄, filtered and concentrated in vacuum to give a crude product. The crude product was purified by silica gel chromatography (gradient: petroleum ether/ethyl acetate) to provide compound A-4 (26.0 g, 74.1 mmol, 84% yield) was obtained as yellow solid.

LC/MS (ES+): m/z=351.1 (M+H)+.

¹H NMR (400 MHZ, DMSO-d6): δ 10.88 (s, 1H), 8.18 (s, 1H), 7.66-7.63 (m, 1H), 7.41-7.39 (m, 1H), 7.18-7.14 (m, 1H), 6.84 (s, 1H), 3.87 (s, 3H), 3.76 (s, 3H), 2.99 (d, J=5.2 Hz, 3H)

### General Procedure for Preparation of Compound A-5

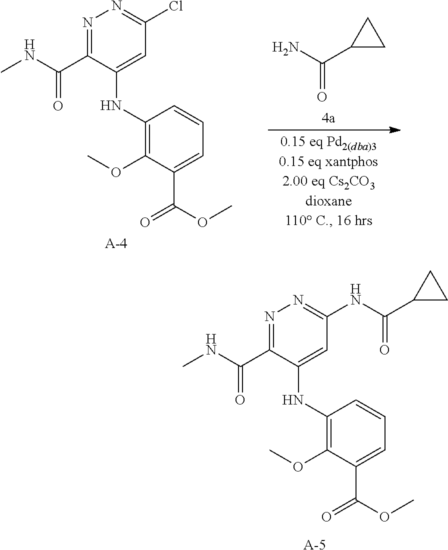

To solution of compound A-4 (25.0 g, 71.2 mmol, 1.00 eq) and cyclopropanecarboxamide (12.1 g, 142 mmol, 2.00 eq) in dioxane (500 mL) was added Cs₂CO₃ (46.4 g, 142 mmol, 2.00 eq), Pd₂(dba)₃ (9.79 g, 10.6 mmol, 0.15 eq) and Xantphos (6.19 g, 10.6 mmol, 0.15 eq). The mixture was then stirred at 110° C. for 16 hrs after which the reaction was determined to be complete by LC/MS analysis. The mixture was filtered through celite and washed with ethyl acetate (500 mL×3), the filtrate was concentrated under reduced pressure. The residue was purified by trituration with petroleum ether:ethyl acetate (1:1 v/v, 100 mL) at 20° C. for 30 mins, and then filtered to give a yellow solid. Compound A-5 (18.7 g, 46.8 mmol, 65.6% yield) was obtained as yellow solid.

LCMS: m/z=400.2 (M+H)+.

¹H NMR (400 MHZ, CDCl₃): δ 11.21 (s, 1H), 9.81-9.77 (m, 1H), 8.21 (s, 1H), 8.03 (d, J=4.8 Hz, 1H), 7.66-7.62 (m, 2H), 7.27-7.23 (m, 1H), 3.93 (s, 3H), 3.88 (s, 3H), 3.05 (d, J=5.2 Hz, 3H), 1.82-1.77 (m, 1H), 1.13-1.09 (m, 2H), 0.97-0.93 (m, 2H).

### General Procedure Preparation of Intermediate A

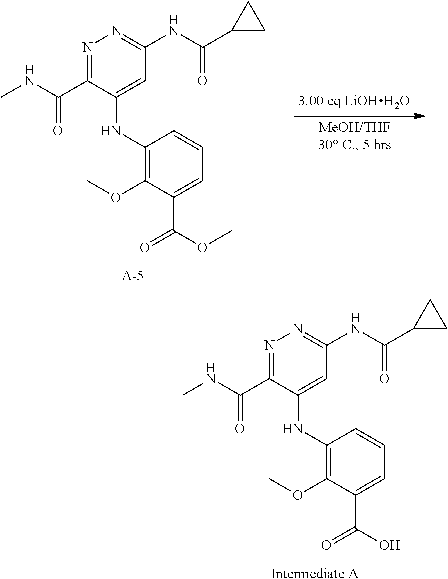

To a solution of compound A-5 (15.0 g, 37.5 mmol, 1.00 eq) in THF (150 mL) and MeOH (45 mL) was added LiOH·H₂O (4.73 g, 112 mmol, 3.00 eq). The mixture was the stirred at 30° C. for 5 hrs until the reaction was determined to be complete by LC/MS analysis. The reaction was adjusted to pH=6 with 1 N HCl, then filtered to give a yellow solid filter cake. Intermediate A (11.2 g, 28.1 mmol, 74.9% yield) was obtained as a yellow solid.

LCMS: m/z=386.1 (M+H)+.

¹H NMR (400 MHZ, DMSO-d6): δ 13.09 (s, 1H), 11.33 (s, 1H), 10.94 (s, 1H), 9.18-9.11 (m, 1H), 8.10 (s, 1H), 7.63-7.61 (m, 1H), 7.51-7.49 (m, 1H), 7.27-7.23 (m, 1H), 3.74 (s, 3H), 2.86 (d, J=4.8 Hz, 3H), 2.09-2.06 (m, 1H), 0.83-0.81 (m, 4H),

### Preparation of Intermediate B

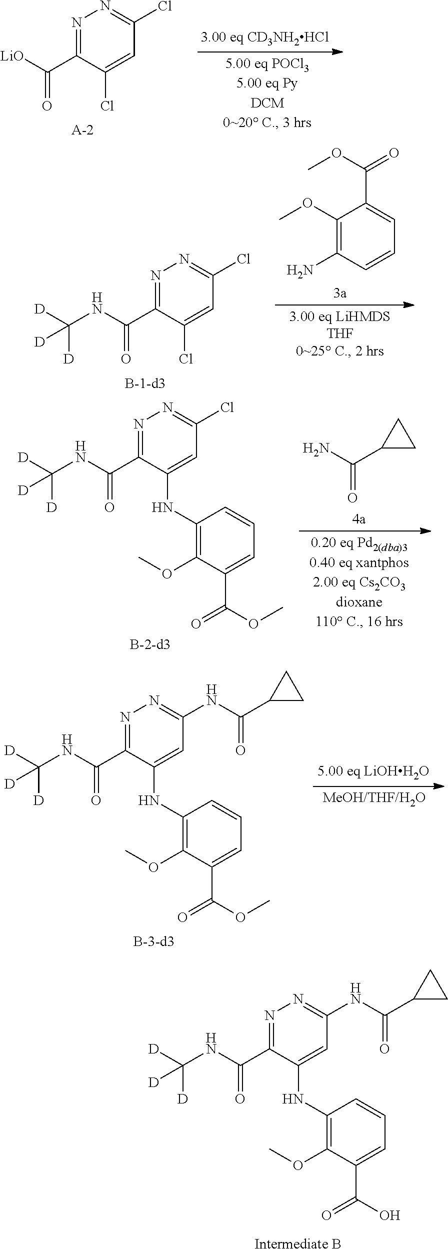

### General Procedure for Preparation of Compound B-1-d3

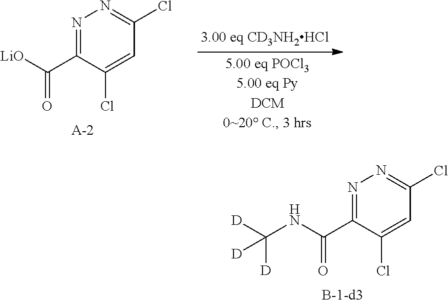

To a solution of compound A-2 (30.0 g, 150 mmol, 1.00 eq) and CD₃NH₂·HCl (26.5 g, 377 mmol, 2.50 eq) in DCM (300 mL) was added pyridine (59.6 g, 754 mmol, 60.8 mL, 5.00 eq), followed by the drop-wise addition of POCl₃ (115 g, 754 mmol, 70.0 mL, 5.00 eq) at 0° C. The mixture was stirred at 25° C. for 3 hrs after which it was determined to be complete by LC/MS analysis. The reaction mixture was poured into H₂O (1.00 L), and the aqueous phase was extracted with dichloromethane (1.00 L×2). The combined organic phase was washed with saturated NaHCO₃ solution (1.00 L×2) and brine (1.00 L×2), dried with anhydrous Na₂SO₄, filtered and concentrated in vacuum to give a crude product. The residue was purified by trituration with petroleum ether/ethyl acetate (10:1 v/v, 200 mL) at 20° C. for 30 mins, and filtered to provide compound B-1-d3 as yellow solid (24.2 g, 115 mmol, 76.8% yield).

LCMS: m/z=209.1 (M+H)+

¹H NMR (400 MHZ, CDCl₃): δ 7.75 (s, 1H), 7.72 (s, 1H)

### General Procedure for Preparation of Compound B-2-d3

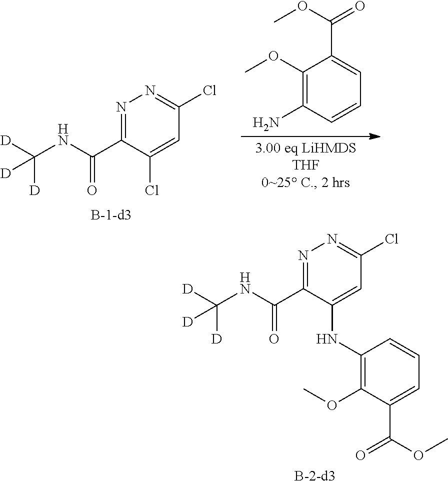

To a solution of compound B-1-d3 (24.0 g, 114 mmol, 1.10 eq) and methyl 3-amino-2 methoxybenzoate (18.9 g, 104 mmol, 1.00 eq) in THF (480 mL) was added LiHMDS (1 M, 313 mL, 3.00 eq) at 0° C. under N₂. The mixture was then stirred at 25° C. for 2 hrs until the reaction was determined to be complete by LC/MS analysis. The reaction mixture was poured into ice-cold saturated NH₄Cl solution (1.00 L), and the aqueous phase was extracted with ethyl acetate (1.00 L×2). The combined organic phase was washed with brine (1.00 L×2), dried with anhydrous Na₂SO₄, filtered and concentrated in vacuum to give a crude product which was purified by silica gel chromatography (gradient of petroleum ether/ethyl acetate: 40:1 to 1:1 v/v) to provide compound B-2-d3 (26.0 g, 73.4 mmol, 70% yield).

LCMS: m/z=354.1 (M+H)+

¹H NMR (400 MHZ, CDCl₃): δ 10.94 (s, 1H), 8.24 (s, 1H), 7.73-7.71 (m, 1H), 7.48-7.47 (m, 1H), 7.25-7.23 (m, 1H), 6.92 (s, 1H), 3.95 (s, 3H), 3.84 (d, J=4.0 Hz, 3H).

### General Procedure for Preparation of Compound B-2-d3

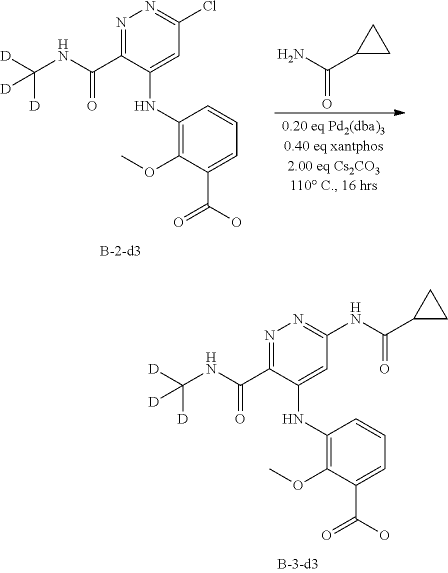 [SMILES: [2H]C([2H])([2H])NC(=O)c1nnc(Cl)cc1Nc1cccc(C(=O)O)c1OC ; NC(=O)C1CC1 ; [2H]C([2H])([2H])NC(=O)c1nnc(NC(=O)C2CC2)cc1Nc1cccc(C(=O)O)c1OC]

To solution of methyl compound B-2-d3 (26.0 g, 73.4 mmol, 1.00 eq) and cyclopropanecarboxamide (12.5 g, 146 mmol, 2.00 eq) in dioxane (1.04 L) was added Cs₂CO₃ (47.8 g, 146 mmol, 2.00 eq), Pd₂(dba)₃ (13.4 g, 14.7 mmol, 0.20 eq) and Xantphos (17.0 g, 29.4 mmol, 0.40 eq). The mixture was then stirred at 110° C. for 16 hrs under N₂ after which the reaction was determined to be complete by LC/MS analysis. The reaction mixture was then poured into H₂O (2.00 L), the aqueous phase was extracted with ethyl acetate (1.00 L×2). The combined organic phase was washed with brine (2.00 L×2), dried with anhydrous Na₂SO₄, filtered and concentrated in vacuum to give a crude product which was purified by trituration with petroleum ether:ethyl acetate (1:1 v/v, 200 mL) at 20° C. for 30 mins, and filtered to provide compound B-3-d3 (19.0 g, 47.2 mmol, 64.2% yield).

LCMS: m/z=403.2 (M+H)+

¹H NMR (400 MHZ, DMSO-d6): δ 11.33 (s, 1H), 10.96 (s, 1H), 9.14 (s, 1H), 8.09 (s, 1H), 7.67-7.65 (m, 1H), 7.52-7.50 (m, 1H), 7.30-7.28 (m, 1H), 3.85 (s, 3H), 3.73 (s, 3H), 2.08-2.05 (m, 1H), 0.85-0.81 (m, 4H).

### General Procedure for Preparation of Intermediate B

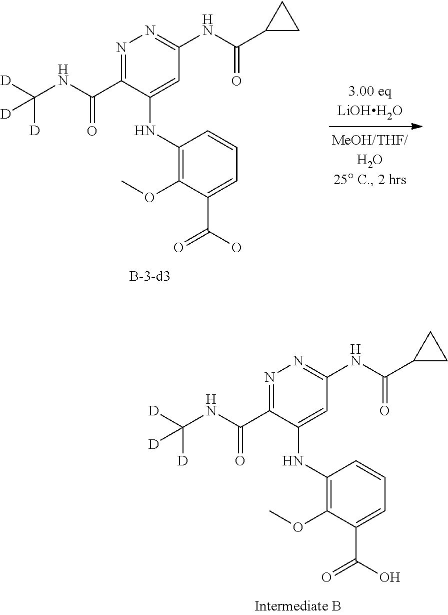 [SMILES: [2H]C([2H])([2H])NC(=O)c1nnc(NC(=O)C2CC2)cc1Nc1cccc(C(=O)O)c1OC ; [2H]C([2H])([2H])NC(=O)c1nnc(NC(=O)C2CC2)cc1Nc1cccc(C(=O)O)c1OC]

To a solution of compound B-3-d3 (19.0 g, 47.2 mmol, 1.00 eq) in THF (285 mL) and MeOH (285 mL), was added LiOH·H₂O (5.94 g, 141 mmol, 3.00 eq) in H₂O (140 mL) at 25° C., the mixture was stirred at 25° C. for 2 hrs after which the reaction was determined to be complete by LC/MS analysis. The mixture was then adjusted to pH about 4 with HCl (1 N) at 0° C. and stirred at 0° C. for 10 mins, concentrated in vacuum to remove THF and MeOH. The resulting solid was filtered to give a yellow solid which was was triturated with MeCN (50.0 mL) at 25° C. for 30 mins and filtered to provide Intermediate B as a yellow solid (9.50 g, 22.5 mmol, 47.8% yield).

LCMS: m/z=389.1 (M+H)+

¹H NMR (400 MHZ, DMSO-d6): δ 13.09 (s, 1H), 11.32 (s, 1H), 10.94 (s, 1H), 9.13 (s, 1H), 8.10 (s, 1H), 7.63-7.61 (m, 1H), 7.51-7.49 (m, 1H), 7.27-7.25 (m, 1H), 3.74 (s, 3H), 2.10-2.04 (m, 1H), 0.82-0.81 (m, 4H).

### Preparation of Intermediate C

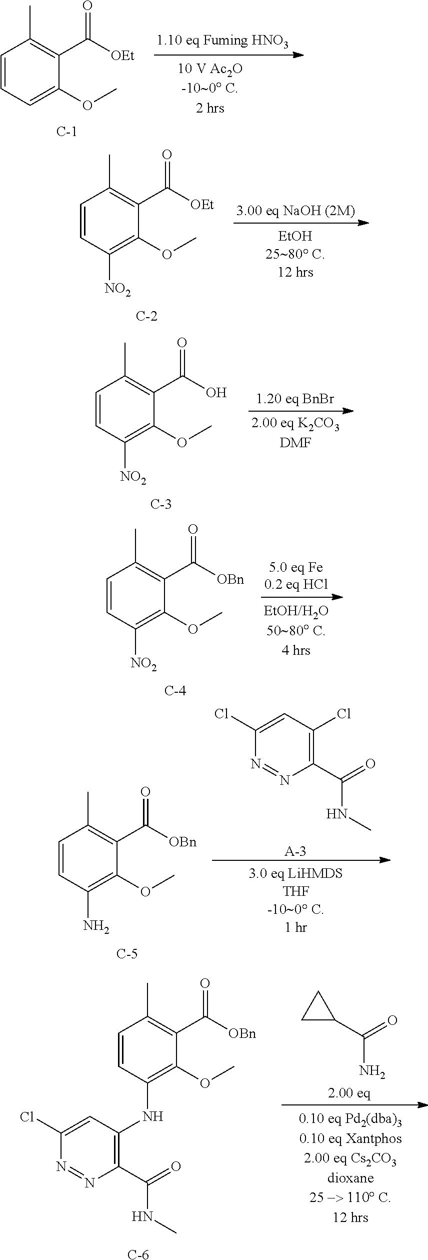

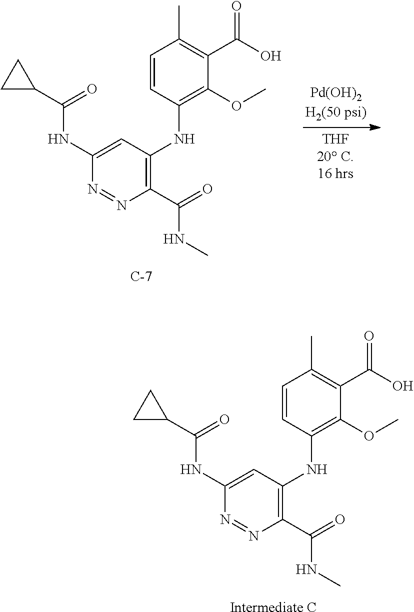

### General Procedure for Preparation of Compound C-2

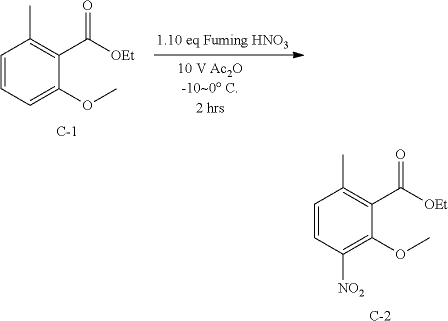

To a solution of compound C-1 (88.0 g, 453 mmol, 1.00 eq) in Ac₂O (880 mL) was added fuming HNO₃ (35.3 g, 561 mmol, 1.24 eq) drop wise at −10° C. The mixture was stirred at −10° C. for 2 hrs after which the reaction was determined to be complete by LC/MS analysis. The reaction was run in two batches combined worked up together. The mixture was poured into ice water (1.00 L) slowly and extracted with ethyl acetate (500 mL×2). The combined organic layer was washed with sat. NaHCO₃ until pH was between 7-8, then washed with brine 10 (500 mL), dried over Na₂SO₄, filtered and concentrated. The residue was purified by column chromatography (gradient of petroleum ether/ethyl acetate) to obtain compound C-2 (122 g, 509 mmol, 56% yield) was obtained as yellow oil.

¹H NMR (400 MHZ, CDCl₃): δ 7.87 (d, J=8.4 Hz, 1H), 7.09 (d, J=8.4 Hz, 1H), 4.47-4.42 (m, 2H), 3.94 (s, 3H), 5.41 (s, 2H), 2.38 (s, 3H), 1.63-1.40 (m, 3H).

### General Procedure for Preparation of Compound C-3

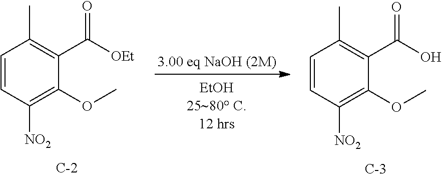

To a solution of compound C-2 (100 g, 418 mmol, 1.00 eq) in EtOH (1.00 L) was added NaOH (2 M, 627 mL, 3.00 eq) at 25° C. after which the mixture was heated to 80° C. and stirred at 80° C. for 12 hrs until determined to be complete by LC/MS analysis. The reaction mixture was filtered and the filtrate was concentrated under vacuum to remove ethanol. The aqueous phase was extracted with ethyl acetate (1.00 L×2) adjusted to pH=2˜4 with 1 N HCl, and extracted with ethyl acetate (2.00 L×2). The combined organic phase was washed with brine (2.00 L×2), dried with anhydrous Na₂SO₄, filtered and concentrated under vacuum to give compound C-3 (90.0 g, crude) as a yellow oil. The crude product was used to the next step.

¹H NMR (400 MHZ, DMSO-d6): δ 7.94 (d, J=8.4 Hz, 1H), 7.27 (d, J=8.4 Hz, 1H), 3.84 (s, 3H), 2.34 (s, 3H).

### General Procedure for Preparation of Compound C-4

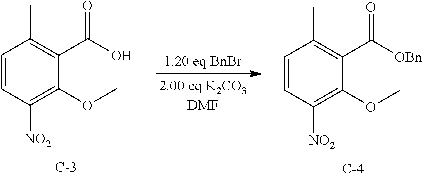

To a solution of compound C-3 (90.0 g, 426 mmol, 1.00 eq) in DMF (900 mL) was added K₂CO₃ (117 g, 852 mmol, 2.00 eq) and BnBr (87.4 g, 511 mmol, 60.7 mL, 1.20 eq), after which the mixture was stirred at 20° C. for 12 hrs until the reaction was determined to be complete by LC/MS analysis. The reaction mixture was poured into H₂O (3.00 L), and the aqueous phase was extracted with ethyl acetate (1.00 L×2) after which the combined organic layer was washed with brine (1.00 L×3), dried over Na₂SO₄, filtered and concentrated to provide compound C-4 (114 g, 378 mmol) as a yellow solid. The crude product was used to the next step.

¹H NMR (400 MHZ, DMSO-d6): δ 8.00 (d, J=8.4 Hz, 1H), 7.48-7.16 (m, 2H), 7.41-7.36 (m, 4H), 7.30-7.28 (m, 1H), 5.41 (s, 2H), 3.74 (s, 3H), 2.28 (s, 3H).

### General Procedure for Preparation of Compound C-5

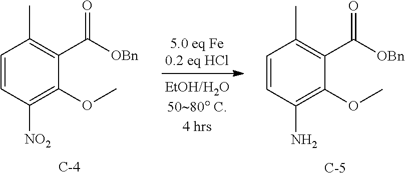

To a solution of compound C-4 (110 g, 365 mmol, 1.00 eq) in EtOH (1.10 L) and H₂O (1.10 L) was added Fe (101 g, 1.83 mol, 5.00 eq), after which HCl (12 M, 6.08 mL, 0.20 eq) was slowly added at 50° C. The mixture was then heated to 80° C. for 16 hrs after which the reaction was determined to be complete by LC/MS analysis. The reaction mixture was then concentrated under vacuum to remove EtOH, the aqueous phase was poured into saturated NaHCO₃ solution (1.00 L), and extracted with ethyl acetate (1.00 L×2). The combined organic phase was washed with brine (1.00 L×2), dried with anhydrous Na₂SO₄, filtered and concentrated under vacuum to give a provide crude compound C-5 (94.0 g, crude) as a yellow oil. The crude product was used to the next step.

LCMS: m/z=272.0 (M+H)+.

1H NMR: (400 MHZ, DMSO-d6) δ 7.46-7.41 (m, 2H), 7.39-7.34 (m, 3H), 6.71-6.66 (m, 2H), 5.32 (s, 2H), 4.89 (s, 2H), 3.57 (s, 3H), 2.02 (s, 3H).

### General Procedure for Preparation of Compound C-6

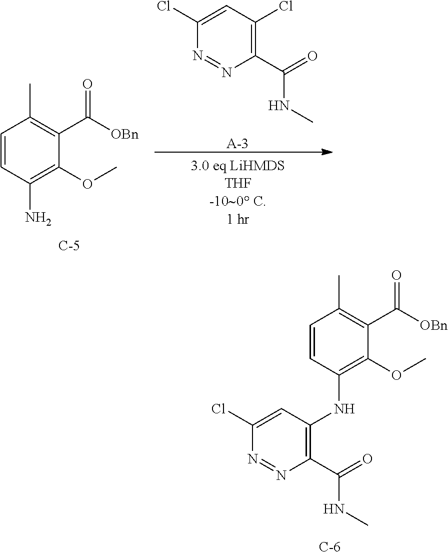

To solution of compound C-5 (60.0 g, 291 mmol, 1.00 eq) and compound 3 (79.0 g, 291 mmol, crude, 1.00 eq) in THF (1.20 L) was added LiHMDS (1 M, 873 mL, 3.00 eq) at −10° C., after which the mixture was warmed to 20° C. and stirred for 1 hr until the reaction was determined to be complete by LC/MS analysis. The reaction mixture was poured into saturated NH₄Cl solution (2.00 L), and the aqueous phase was extracted with ethyl acetate (2.00 L×2). The combined organic phase was washed with brine (2.00 L×2), dried with anhydrous Na₂SO₄, filtered and concentrated in vacuum to give crude product. The residue was purified by trituration with petroleum ether/ethyl acetate (3:1 v/v, 200 mL) at 20° C. for 30 min and filtered after which the filter cake was dried over vacuum to give compound C-6 as a yellow solid (90.0 g, 204 mmol, four steps yield: 49% yield).

LCMS: m/z=440.8 (M+H)+.

¹H NMR (400 MHZ, DMSO-d6): δ 10.8 (s, 1H), 9.39-9.37 (m, 1H), 7.51-7.46 (m, 3H), 7.41-7.37 (m, 3H), 7.12 (d, J=8.4 Hz, 1H), 7.04 (s, 1H), 5.38 (s, 2H), 3.60 (s, 3H), 2.85 (d, J=8.4 Hz, 3H), 2.21 (s, 3H).

### General Procedure for Preparation of Compound C-7

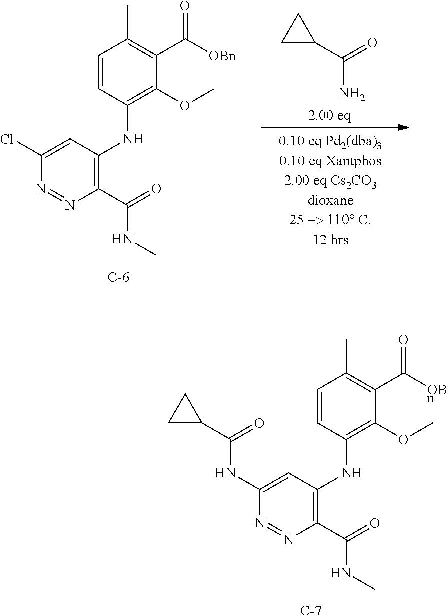

To solution of compound C-6 (70.0 g, 158 mmol, 1.00 eq) and cyclopropanecarboxamide (27.0 g, 317 mmol, 2.00 eq) in dioxane (2.10 L) was added Cs₂CO₃ (103 g, 317 mmol, 2.00 eq), Pd₂(dba)₃ (14.5 g, 15.8 mmol, 0.10 eq) and Xantphos (9.19 g, 15.8 mmol, 0.10 eq) at 25° C. The mixture was degassed with N₂ three times, then the mixture was heated to 110° C. and stirred for 12 hrs under N₂ atmosphere after which the reaction was determined to be complete by LC/MS analysis. The reaction mixture was poured into H₂O (5.00 L), after which the aqueous phase was extracted with ethyl acetate (2.00 L×3). The combined organic phase was washed with brine (3.00 L×2), dried with anhydrous Na₂SO₄, filtered and concentrated under vacuum to give a crude product. The crude product was purified by trituration with ethyl acetate (500 mL) at 20° C. for 30 mins, filtered and the filter cake was dried over vacuum to give compound C-7 (50 g, crude) as a yellow solid. LCMS: m/z=490.2 (M+H)+.

¹H NMR (400 MHZ, DMSO-d6): δ 11.30 (s, 1H), 10.74 (s, 1H), 9.16-9.13 (m, 1H), 8.00 (s, 1H), 7.48-7.42 (m, 2H), 7.40-7.35 (m, 4H), 7.11 (d, J=8.8 Hz, 1H), 5.37 (s, 2H), 3.59 (s, 3H), 2.84 (d, J=4.8 Hz, 3H), 2.20 (s, 3H), 2.07-2.04 (m, 1H), 0.84-0.79 (m, 4H).

### General Procedure for Synthesis of Intermediate C

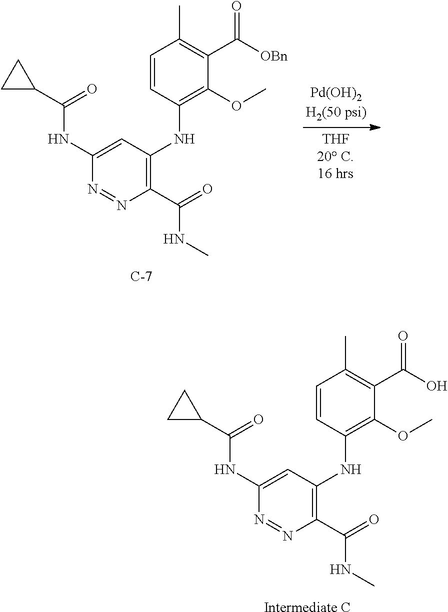

To a solution of compound C-7 (25.0 g, 51.0 mmol, 1.00 eq) in THF (1.00 L) was added Pd(OH)₂ (5.00 g, 20% purity), and the mixture was stirred at 25° C. for 12 hrs under H₂ (50 psi) until the reaction was determined to be complete by LC/MS analysis. The reaction mixture was filtered and the filter cake was washed with DMF (1.00 L×2), then the combined filtrate was concentrated to remove THF. The residue was poured into H₂O (5.00 L) filter cake was collected and triturated with methanol/ethyl acetate (1:1 v/v, 300 mL) at 25° C. for 2 hrs. The resulting filter cake was dried over vacuum to provide intermediate C(two batches combined, 30.0 g, 63.0 mmol, two step yield 56.2% yield) as a yellow solid.

LCMS: m/z=400.0 (M+H)+.

¹H NMR (400 MHZ, DMSO-d6): δ 11.30 (s, 1H), 10.74 (s, 1H), 9.16-9.12 (m, 1H), 8.02 (s, 1H), 7.37 (d, J=8.0 Hz, 1H), 7.11-7.06 (m, 1H), 3.67 (s, 3H), 2.85 (d, J=4.8 Hz, 3H), 2.26 (s, 3H), 2.09-2.04 (m, 1H), 0.82-0.80 (m, 4H).

### Preparation of Intermediate D

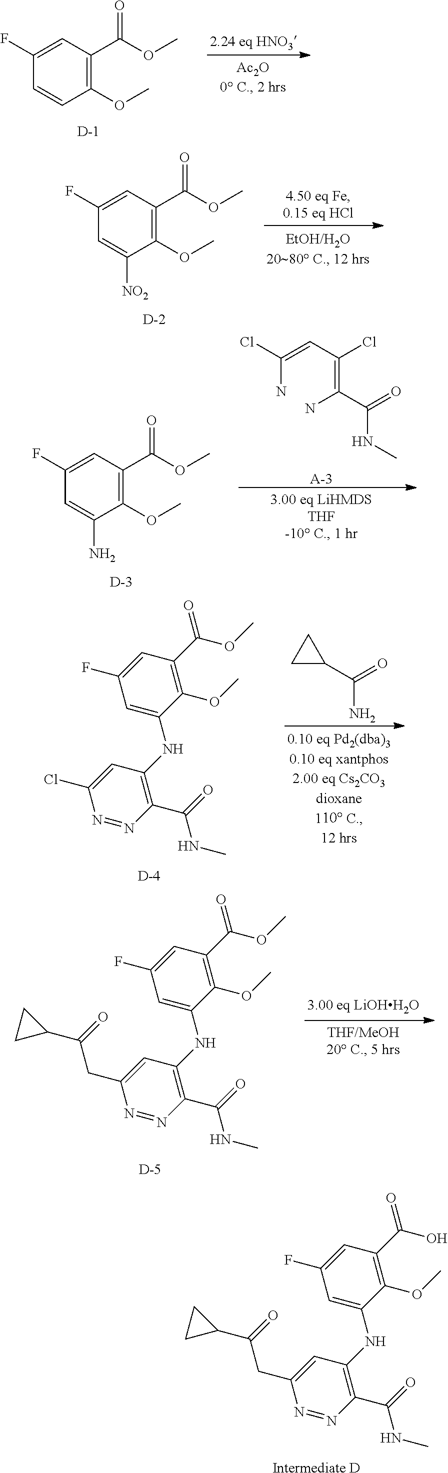

### General Procedure for Preparation of Compound D-2

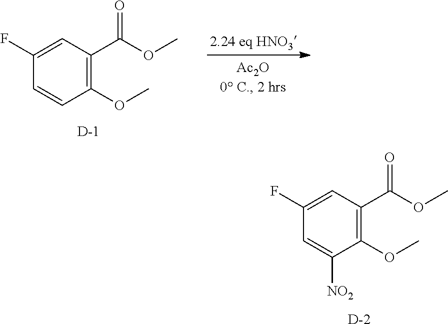

To a mixture of compound D-1 (130 g, 706 mmol, 1.00 eq) in Ac₂O (1.04 L) was dropwise added HNO₃ (99.5 g, 1.58 mol, 71.1 mL, 2.24 eq) at 0° C., after with the mixture was stirred at 20° C. for 2 hrs until the reaction was determined to be complete by HPLC analysis. The mixture was poured into H₂O (1.50 L) extracted with ethyl acetate (500 mL×2), after which the combined organic layers were washed with brine (1.00 L×2), NaOH solution (1.00 mol/L, 1.50 L), brine (1.00 mL×2), and concentrated under reduced pressure to give compound D-2 (100 g, 436 mmol, 61.8% yield) as a brown solid.

¹H NMR (400 MHZ, DMSO-d6): δ 8.22-8.19 (m, 1H), 7.95-7.92 (m, 1H), 3.90 (s, 3H), 3.86 (s, 3H)

### General Procedure for the Preparation of Compound D-3

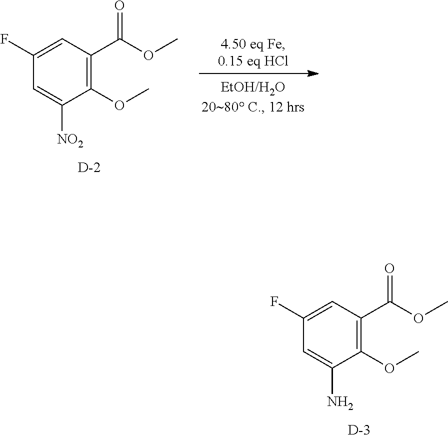

To a mixture of compound D-3 (100 g, 436 mmol, 1.00 eq) in EtOH (1.00 L) was added H₂O (1.00 L), Fe (100 g, 1.79 mol, 4.10 eq) at 20° C., after which HCl was added dropwise (12.0 M, 5.45 mL, 0.15 eq) at 80° C. and stirred for 12 hrs after which the reaction was determined to be complete by LC/MS analysis. The mixture was filtered after which the the filtrate was concentrated to remove EtOH, after which H₂O was added (300 mL) followed by saturated NaHCO₃ solution (300 mL) and extracted with ethyl acetate (500 mL×2). The combined organic layers were washed with brine (500 mL×3) and concentrated under reduced pressure to give compound D-4 (70.0 g, 351 mmol, 80.5% yield) as a brown oil.

¹H NMR (400 MHZ, DMSO-d6): δ 6.66-6.62 (m, 1H), 6.54-6.51 (m, 1H), 5.52 (s, 2H), 3.80 (s, 3H), 3.65 (s, 3H)

### General Procedure for Synthesis of Compound D-4

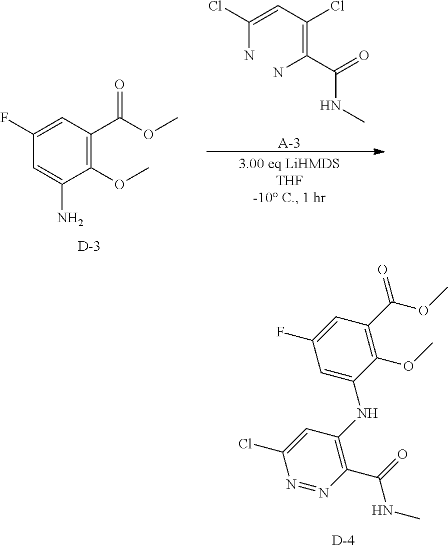

To solution of compound D-3 (50 g, 242 mmol, 1.00 eq) and compound A-3 (48.3 g, 242 mmol, 1.00 eq) in THF (1.00 L) was added LiHMDS (1 M, 728 mL, 3.00 eq) at −10° C., after which the mixture was stirred at 20° C. for 1 hr until the reaction was determined to be complete by LC/MS analysis. The reaction mixture was poured into saturated NH₄Cl solution (2.00 L), and the aqueous phase was extracted with ethyl acetate (2.00 L×2). The combined organic phase was washed with brine (2.00 L×2), dried with anhydrous Na₂SO₄, filtered and concentrated under vacuum to give a crude product. The residue was purified by trituration with petroleum ether/ethyl acetate (1:1 v/v, 200 mL) at 20° C. for 30 mins, and filtered to give compound D-4 as a yellow solid (70.0 g, 189 mmol, 78.2% yield).

HNMR (400 MHZ, DMSO-d6): δ 11.2 (s, 1H), 9.43-9.42 (m, 1H), 7.76-7.73 (m, 1H), 7.43 (s, 1H), 7.35-7.32 (m, 1H), 3.91 (s, 3H), 3.73 (s, 3H), 2.86 (d, J=4.8 Hz, 3H).

### General Procedure for the Preparation of Compound D-5

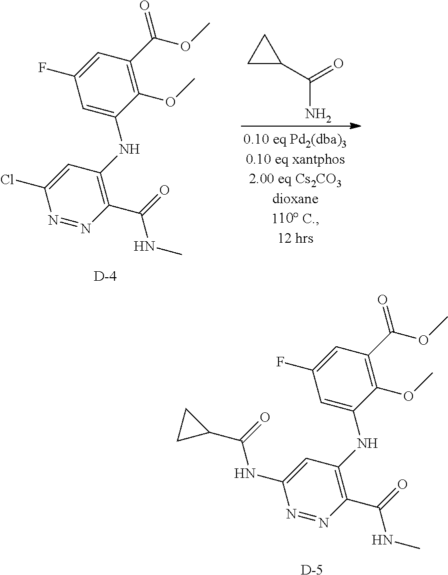

To solution of compound D-4 (70.0 g, 189 mmol, 1.00 eq) and cyclohexanecarboxamide (32.3 g, 379 mmol, 2.00 eq) in dioxane (2.10 L) was added Cs₂CO₃ (123 g, 379 mmol, 2.00 eq), Pd₂(dba)₃ (17.3 g, 18.9 mmol, 0.10 eq) and Xantphos (10.9 g, 18.9 mmol, 0.10 eq). The mixture was stirred at 110° C. for 12 hrs under N₂ atmosphere until it was determined to be complete by LC/MS analysis. The reaction mixture was then poured into H₂O (2.00 L), and the aqueous phase was extracted with ethyl acetate (2.00 L×2). The combined organic phase was washed with brine (2.00 L×2), dried with anhydrous Na₂SO₄, filtered and concentrated in vacuum to give a crude product. The residue was purified by trituration with ethyl acetate (2.00 L) at 20° C. for 30 mins, and then filtered to provide compound D-5 as a yellow solid (43.0 g, 103 mmol, 54.3% yield).

LCMS: m/z=418.0 (M+H)+.

¹H NMR (400 MHZ, DMSO-d6): δ 11.4 (s, 1H), 11.1 (s, 1H), 9.21-9.20 (m, 1H), 8.21 (s, 1H), 7.60-7.57 (m, 1H), 7.29-7.27 (m, 1H), 3.86 (s, 3H), 3.74 (s, 3H), 2.85 (d, J=4.4 Hz, 3H), 2.10-2.07 (m, 1H), 0.85 (d, J=6.0 Hz, 4H)

### General Procedure for Preparation of Intermediate D

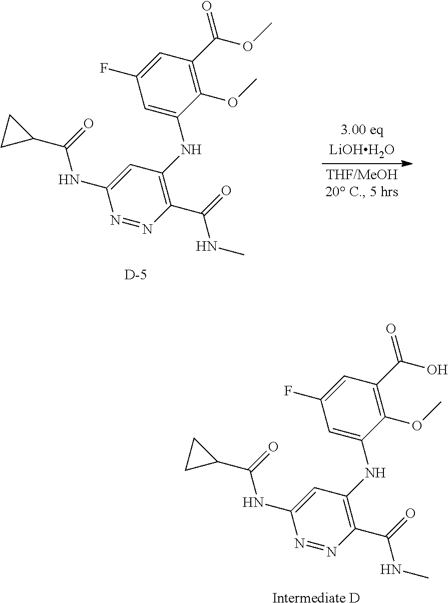

To a solution of compound D-5 (40.0 g, 95.8 mmol, 1.00 eq) in THF (400 mL) and MeOH (120 mL) was added LiOH·H₂O (12.0 g, 287 mmol, 3.00 eq). The mixture was stirred at 20° C. for 4 hrs until the reaction was determined to by complete by LC/MS analysis. The reaction mixture was then poured into H₂O (1.00 L), after which the aqueous phase was extracted with ethyl acetate (1.00 L×3). Then the aqueous phase was adjusted to pH=2˜4 with 1 N HCl, and the resulting precipitate was filtered to provide intermediate D as a yellow solid (20.0 g, 48.0 mmol, 50.1% yield).

LCMS: m/z=404.0 (M+H)+.

¹H NMR (400 MHZ, DMSO-d6): δ 11.3 (s, 1H), 11.1 (s, 1H), 9.21-9.18 (m, 1H), 8.21 (s, 1H), 7.55-7.52 (m, 1H), 7.26-7.23 (m, 1H), 3.74 (s, 3H), 2.85 (d, J=4.8 Hz, 3H), 2.10-2.06 (m, 1H), 0.85 (d, J=6.0 Hz, 4H).

19F NMR (400 MHZ, DMSO-d6): δ 115.583 ppm

### Preparation of Intermediate E

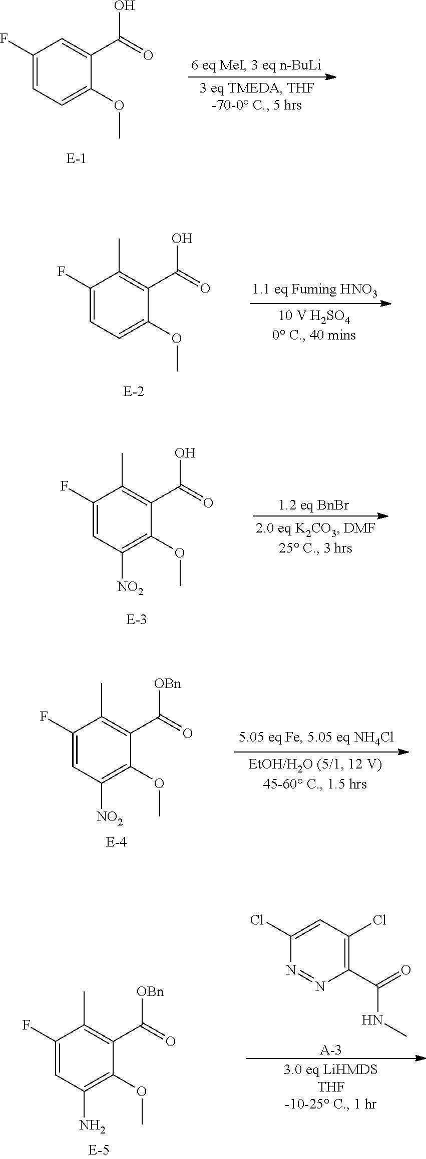

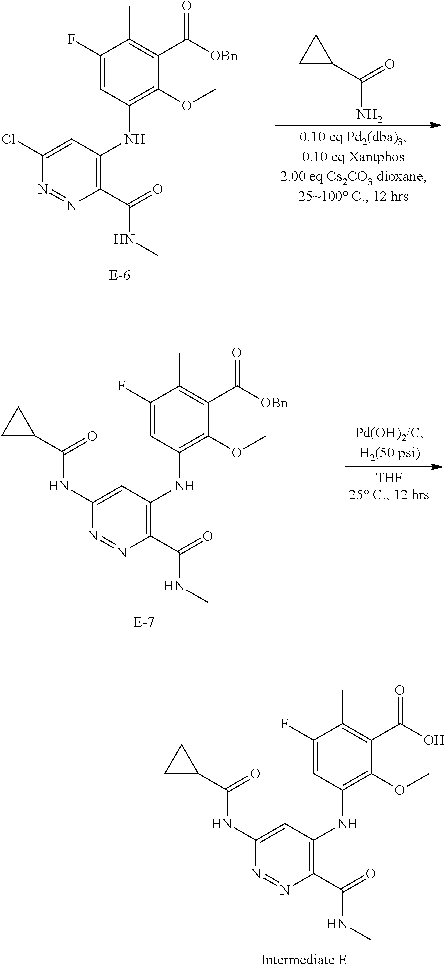 [SMILES: CNC(=O)c1nnc(Cl)cc1Nc1cc(F)c(C)c(C(=O)OCc2ccccc2)c1OC ; NC(=O)C1CC1 ; CNC(=O)c1nnc(NC(=O)C2CC2)cc1Nc1cc(F)c(C)c(C(=O)OCc2ccccc2)c1OC ; CNC(=O)c1nnc(NC(=O)C2CC2)cc1Nc1cc(F)c(C)c(C(=O)O)c1OC]

### General Procedure for Synthesis of Compound E-2

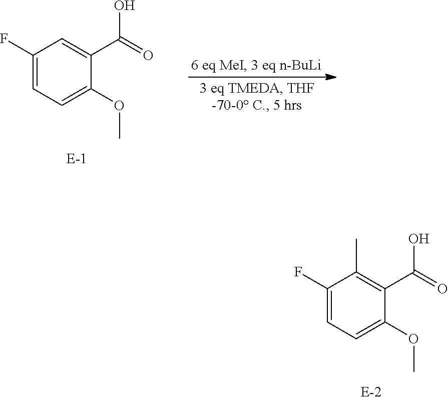

To a mixture of compound E-1 (100 g, 588 mmol, 1.00 eq) and TMEDA (205 g, 1.76 mol, 266 mL, 3.00 eq) in THF (1.50 L) was added n-BuLi (2.50 M, 705 mL, 3.00 eq) dropwise at −70° C., then the mixture was stirred at −70° C. for 1 hr. Additional MeI (500 g, 3.53 mol, 219 mL, 6.00 eq) was drop-wise added to the mixture at −70° C. and the mixture was stirred at −70° C. for 2 hrs. after which the resulting mixture was warmed to 0° C. and stirred at 0° C. for 2 hrs after which the reaction was determined to be complete by LC/MS analysis. The mixture was then poured into ice-water (5.00 L) and extracted with dichloromethane (1.00 L×3). The aqueous phase was adjusted to pH about 3 with HCl (1.00 M) and extracted with ethyl acetate (2.00 L×3). The combined organic layer was dried over Na₂SO₄, filtered and concentrated under reduced pressure to obtain compound E-2 (110 g, crude) as brown oil.

LCMS: m/z=183.0 (M−H)−

¹H NMR 400 MHZ, CDCl3: δ 7.07 (t, J=8.8 Hz, 1H), 6.77 (dd, J=9.2, 4.0 Hz, 1H), 3.87 (s, 3H), 2.35 (d, J=1.2 Hz, 3H).

### General Procedure for Preparation of Compound E-3

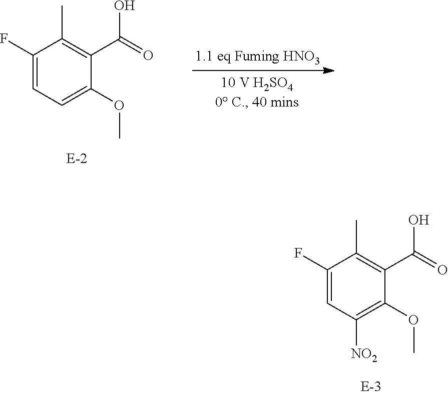 [SMILES: COc1ccc(F)c(C)c1C(=O)O ; COc1c([N+](=O)[O-])cc(F)c(C)c1C(=O)O]

A mixture of compound E-2 (93.0 g, 505 mmol, 1.00 eq) in H₂SO₄ (840 mL) was stirred at 0° C. for 20 mins until dissolving, then a solution of fuming HNO₃ (35.0 g, 555 mmol, 1.10 eq) in H₂SO₄ (95.0 mL) was added drop-wise to the mixture at 0° C. The mixture was stirred at 0° C. for 20 mins after which the reaction was determined to be complete by HPLC analysis. The mixture was poured into ice-water (2.50 L) slowly under stirring and extracted with ethyl acetate (1.00 L×3), after which the combined organic phase was washed with brine (500 mL×2), dried with anhydrous Na₂SO₄, filtered and concentrated under reduced pressure to obtain compound 20 (110 g, crude) as brown oil. The crude material was purified by reverse-phase HPLC (column: Phenomenex luna C18, mobile phase (gradient: 0.01% formic acid in water/MeCN). The eluent was concentrated under reduced pressure to remove MeCN, then extracted with 3thyl acetate (200 mL×3), after which the combined the organic layer was washed with brine (100 mL), dried over Na₂SO₄, filtered and concentrated under reduced pressure to afford compound E-3 (8.45 g, 37.12 mmol, 98.9% purity) as a gray solid.

LCMS: m/z=228.0 (M−H)−

¹H NMR (400 MHZ, DMSO-d6): δ 8.00 (d, J=9.2 Hz, 1H), 3.83 (s, 3H), 2.23 (d, J=2.4 Hz, 3H)

### General Procedure for Preparation of Compound E-4

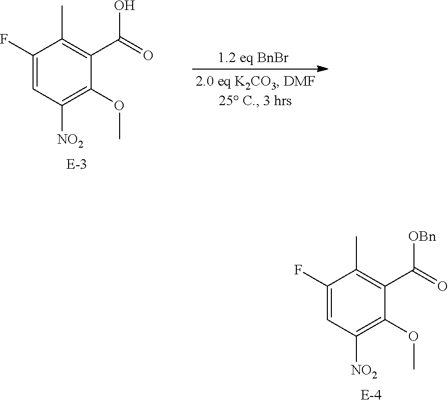 [SMILES: COc1c([N+](=O)[O-])cc(F)c(C)c1C(=O)O ; COc1c([N+](=O)[O-])cc(F)c(C)c1C(=O)OCc1ccccc1]

To a mixture of compound E-3 (130 g, 567 mmol, 1.00 eq) and BnBr (116 g, 681 mmol, 80.8 mL, 1.20 eq) in DMF (1.30 L) was added K₂CO₃ (157 g, 1.13 mol, 2.00 eq) at 25° C. Then the mixture was stirred at 25° C. for 3 hrs until the reaction was determined to be complete by LC/MS analysis. The mixture was diluted with MTBE (1.50 L) and filtered through celite, and the filter cake was washed with MTBE (400 mL×3). The combined organic phase was separated, washed with brine (500 mL×2), dried over Na₂SO₄, filtered and concentrated under reduced pressure. The residue was purified by silica gel flash column chromatography (gradien petroleum ether/ethyl acetate) to obtain compound E-4 (91.0 g, 282 mmol, 50% yield) as a yellow oil.

LCMS: m/z=318.1 (M−H)−

¹H NMR (400 MHZ, CDCl₃): δ 7.68 (d, J=8.4 Hz, 1H), 7.47-7.39 (m, 5H), 5.42 (s, 2H), 3.81 (s, 3H), 2.23 (d, J=1.2 Hz, 3H).

### General Procedure for Preparation of Compound E-5

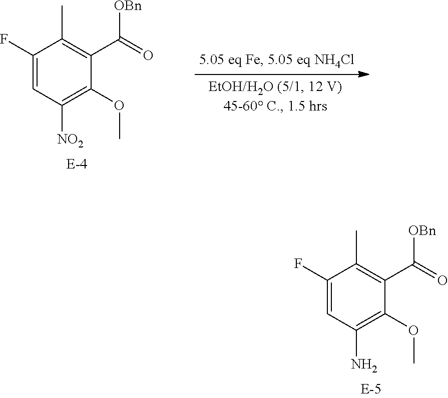 [SMILES: COc1c([N+](=O)[O-])cc(F)c(C)c1C(=O)OCc1ccccc1 ; COc1c(N)cc(F)c(C)c1C(=O)OCc1ccccc1]

To a mixture of compound E-4 (91.0 g, 282 mmol, 1.00 eq) and NH₄Cl (76.2 g, 1.43 mol, 5.05 eq) in EtOH (910 mL) and H₂O (182 mL) was added Fe (79.6 g, 1.42 mol, 5.05 eq) slowly in batches at 45° C. The mixture was then heated to 60° C. and stirred at 60° C. for 1.5 hrs until the reaction was determined to be complete by TLC analysis. The mixture was diluted with ethyl acetate (1.00 L), then filtered and the filter cake was washed with ethyl acetate (300 mL×2). The combined the organic layer and washed with H₂O (300 mL×2), dried over Na₂SO₄, filtered and concentrated under reduced pressure. The residue was purified by flash siliga gel column chromatography (gradient petroleum ether/ethyl acetate) to obtain the crude product. The crude product was triturated with petroleum ether/ethyl acetate (20:1 v/v, 100 mL) at 25° C. for 30 mins. The mixture was filtered and the filter cake was dried over vacuum to obtain compound E-5 (53.0 g, 175 mmol, 62% yield) as a yellow solid.

LCMS: m/z=290.2 (M+H)+

¹H NMR (400 MHZ, CDCl3) δ 7.49-7.44 (m, 2H), 7.42-7.34 (m, 3H), 6.48 (d, J=10.8 Hz, 1H), 5.39 (s, 2H) 3.81 (br s, 2H), 3.67 (s, 3H), 2.05 (d, J=2.0 Hz, 3H).

### General Procedure for Synthesis of Compound E-6

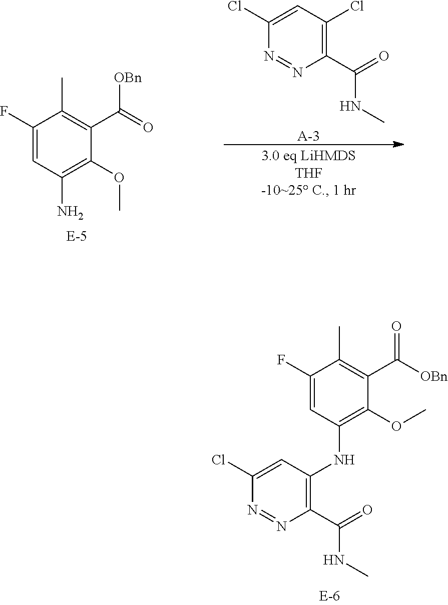 [SMILES: COc1c(N)cc(F)c(C)c1C(=O)OCc1ccccc1 ; CNC(=O)c1nnc(Cl)cc1Cl ; "A-3" ; CNC(=O)c1nnc(Cl)cc1Nc1cc(F)c(C)c(C(=O)OCc2ccccc2)c1OC]

To a solution compound E-5 (36.0 g, 174 mmol, 1.00 eq) and compound A-3 (50.5 g, 174 mmol, 1.00 eq) in THF (180 mL) was drop wise added LiHMDS (1 M, 524 mL, 3.00 eq) at −10° C., and the mixture was allowed to warm to 25° C. and stirred at 25° C. for 1 hr until the reaction was determined to be complete by LC/MS analysis. The reaction mixture was then poured into saturated NH₄Cl solution (2.00 L), after which the aqueous phase was extracted with ethyl acetate (2.00 L×2). The combined organic phase was washed with brine (2.00 L*2), dried with anhydrous Na₂SO₄, filtered and concentrated in vacuum to give a crude compound E-6 (80.0 g, crude) as a brown solid.

LCMS: m/z=459.1 (M+H)+.

1H NMR (400 MHz, CDCl₃): δ 10.89 (s, 1H), 8.26 (d, J=4.0 Hz, 1H), 7.47-7.36 (m, 5H), 7.04 (d, J=9.6 Hz, 1H), 6.93 (s, 1H), 5.41 (s, 2H), 3.63 (s, 3H), 3.06 (d, J=5.2 Hz, 3H), 2.19 (s, 3H).

### General Procedure for the Synthesis of Compound E-7

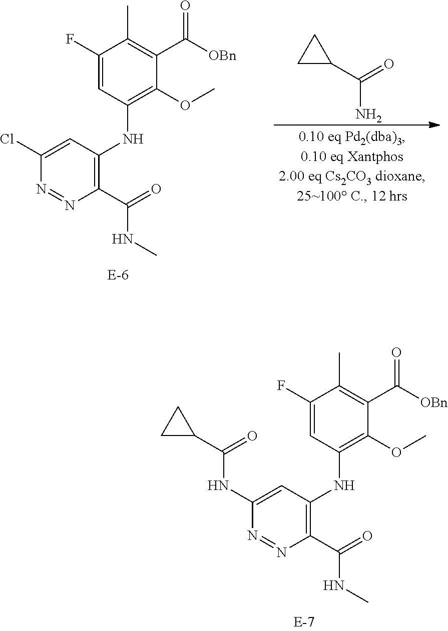 [SMILES: CNC(=O)c1nnc(Cl)cc1Nc1cc(F)c(C)c(C(=O)OCc2ccccc2)c1OC ; NC(=O)C1CC1 ; CNC(=O)c1nnc(NC(=O)C2CC2)cc1Nc1cc(F)c(C)c(C(=O)OCc2ccccc2)c1OC]

To a solution of compound E-6 (80.0 g, 174 mmol, 1.00 eq) and cyclopropancecarboxamide (29.6 g, 348 mmol, 2.00 eq) in dioxane (2.40 L) was added Cs₂CO₃ (113 g, 348 mmol, 2.00 eq), Pd₂(dba)₃ (15.9 g, 17.4 mmol, 0.10 eq) and Xantphos (10.0 g, 17.4 mmol, 0.10 eq) at 25° C., after which the mixture was degassed and purged with N₂ for 3 times. Then the mixture was heated to 100° C. and stirred at 100° C. for 12 hrs under N₂ atmosphere until the reaction was determined to be complete by LC/MS analysis. The reaction mixture was then cooled to 25° C. and poured into H₂O (5.00 L), after which the aqueous phase was extracted with ethyl acetate (3.00 L×3). The combined organic phase was washed with brine (3.00 L×3), dried with anhydrous Na₂SO₄, filtered and concentrated in vacuum to give a crude residue. The residue was triturated with ethyl acetate (500 mL) at 20° C. for 2 hrs, filtered and the filter cake was dried over vacuum at 50° C. to provide compound E-7 (58.0 g, 111 mmol) as a yellow solid.

LCMS: m/z=508.2 (M+H)+.

¹H NMR (400 MHZ, DMSO-d6): δ 11.37 (s, 1H), 10.292 (s, 1H), 9.21-9.17 (m, 1H), 8.10 (s, 1H), 7.49-7.38 (m, 6H), 5.40 (s, 2H), 3.59 (s, 3H), 2.84 (d, J=4.8 Hz, 3H), 2.10 (s, 3H), 2.08-2.05 (m, 1H), 0.84-0.82 (m, 4H).

### General Procedure for the Synthesis of Intermediate E

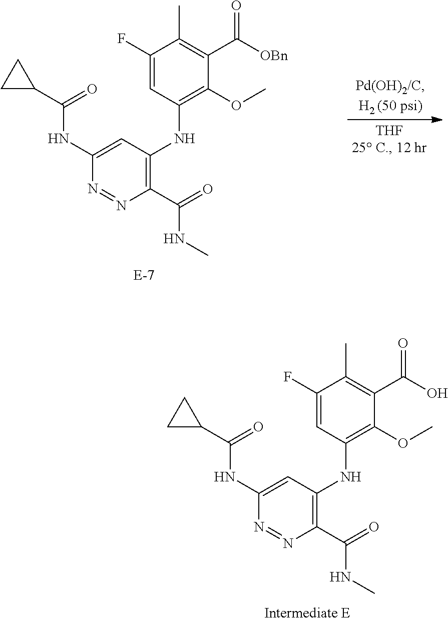 [SMILES: CNC(=O)c1nnc(NC(=O)C2CC2)cc1Nc1cc(F)c(C)c(C(=O)OCc2ccccc2)c1OC ; CNC(=O)c1nnc(NC(=O)C2CC2)cc1Nc1cc(F)c(C)c(C(=O)O)c1OC]

To a solution of compound E-7 (25.0 g, 48.0 mmol, 1.00 eq) in THF (1.00 L) was added Pd(OH)₂/C (2.50 g, 20% purity) at 25° C., the mixture was degassed and purged with H₂ for 3 times. The mixture was stirred under H₂ (50 Psi) at 25° C. for 12 hrs until the reaction was determined to be complete by LC/MS analysis. The reaction mixture was filtered, then the filter cake was washed with DMF (1.00 L×3), then combined filtrate was concentrated in vacuum to remove THF. The residue was poured into H₂O (6.00 L), and the resulting precipitate was filtered to give a filter cake. The filter cake was triturated with ethyl acetate/methanol (1:1 v/v, 300 mL) at 20° C. for 30 mins, filtered and the filter cake was dried over vacuum at 50° C. to provide Intermediate E (25.01 g, 57.1 mmol, three steps yield, combination of two batches: 34% yield) as a yellow solid.

LCMS: m/z=417.9 (M+H)+.

1H NMR: EW30932-2-P1A (400 MHZ, DMSO-d6) δ 11.37 (s, 1H), 10.92 (s, 1H), 9.20-9.18 (m, 1H), 8.11 (s, 1H), 7.37 (d, J=10.8 Hz, 1H), 3.68 (s, 3H), 2.85 (d, J=4.8 Hz, 3H), 2.16 (s, 3H), 2.10-2.07 (m, 1H), 0.84-0.83 (m, 4H).

19F NMR: EW30932-2-P1A (400 MHZ, DMSO-d6) δ−118.425 ppm.

### Preparation of Intermediate F

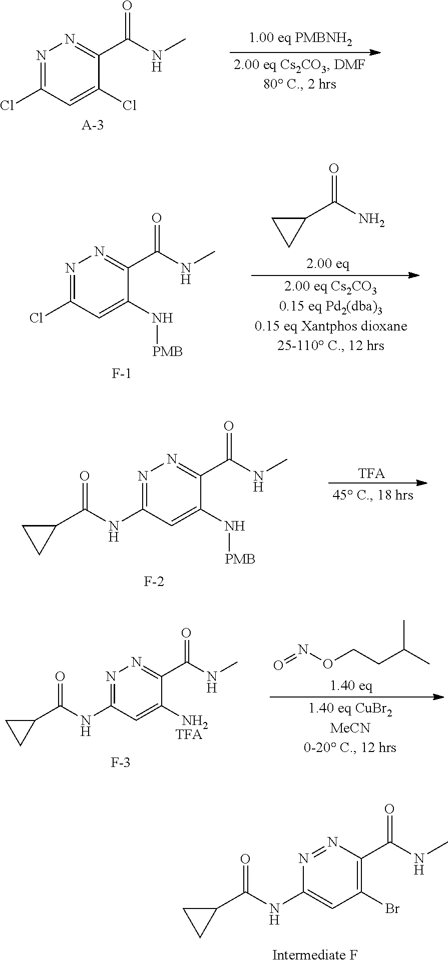

### General Procedure for Synthesis of Compound F-1

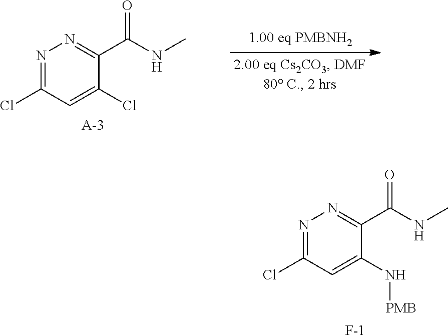

To a solution of compound A-3 (140 g, 680 mmol, 1.00 eq) and 4-methoxylbenzylamine (93.2 g, 680 mmol, 87.9 mL, 1.00 eq) in DMF (1.40 L) was added Cs₂CO₃ (443 g, 1.36 mol, 2.00 eq). The mixture was then stirred at 80° C. for 2 hrs after which the reaction was determined to be complete by LC/MS analysis. The mixture was then poured into H₂O (6.00 L). The resulting filtrate was dissolved with DCM (2.50 L) and the organic phase was washed with brine (1.50 L×2), dried with anhydrous Na₂SO₄, filtered and concentrated in vacuum to give compound F-1 (173 g, 564 mmol, 83% yield) as a brown solid.

LCMS: m/z=307.1 (M+H)+

¹H NMR (400 MHZ, DMSO-d6): δ 9.27-9.30 (m, 1H), 9.14 (d, J=4.8 Hz, 1H), 7.28 (d, J=8.4 Hz, 2H), 7.03 (s, 1H), 6.92 (d, J=8.8 Hz, 2H), 4.42 (d, J=6.0 Hz, 2H), 3.73 (s, 3H), 2.78 (d, J=4.8 Hz, 3H).

### General Procedure for Preparation of Compound F-2

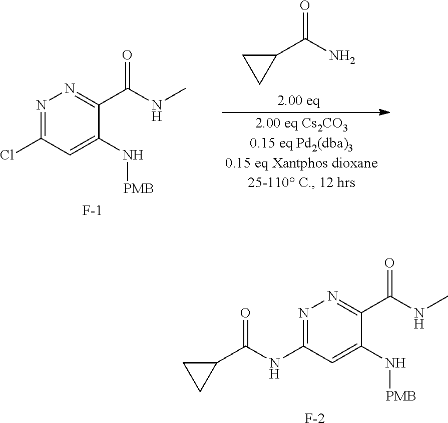

To a solution of compound F-1 (57.5 g, 187 mmol, 1.00 eq) in dioxane (1725 mL) was added compound cyclopropylcarboxamide (31.9 g, 375 mmol, 2.00 eq) and Cs₂CO₃ (122 g, 375 mmol, 2.00 eq) at 25° C. under N₂ after which Pd₂(dba)₃ (25.8 g, 28.1 mmol, 0.15 eq) and Xantphos (16.3 g, 28.1 mmol, 0.15 eq) was added to the mixture under N₂. The mixture was then stirred at 110° C. for 12 hrs under N₂ until the reaction was determined to be complete by LC/MS analysis. The mixture was then filtered and the filter cake was washed with ethyl acetate (2000 mL). The mixture was poured into H₂O (3000 mL) and extracted with ethyl acetate (1500 mL×2). The combined organic phase was washed with brine (2000 mL), dried over anhydrous Na₂SO₄, filtered and concentrated under vacuo. The crude product was triturated with ethyl acetate at 25° C. for 30 mins to give compound F-2 (123 g, 346 mmol, 61.5% yield) as a yellow solid.

LCMS: m/z=356.1 (M+H)+

¹H NMR (400 MHZ, DMSO-d6): δ 11.1 (s, 1H), 9.10-9.13 (m, 1H), 8.90-8.93 (m, 1H), 7.63 (s, 1H), 7.26 (d, J=8.8 Hz, 2H), 6.91 (d, J=8.8 Hz, 2H), 4.30 (d, J=5.6 Hz, 2H), 3.73 (s, 3H), 2.78 (d, J=4.8 Hz, 3H), 2.04-2.10 (m, 1H), 0.82 (d, J=6.0 Hz, 4H).

### General Procedure for Synthesis of Compound F-3

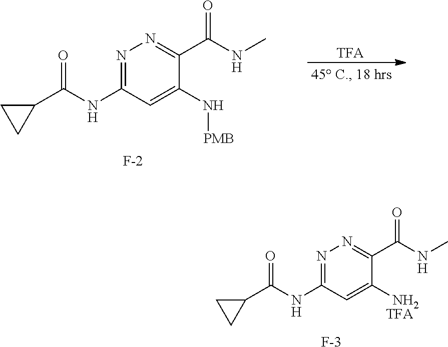

To the mixture of compound F-2 (154 g, 433 mmol, 1.00 eq) was added TFA (1230 mL). The mixture was stirred at 45° C. for 18 hrs after which the reaction was determined to be complete by LC/MS analysis. The mixture was concentrated in vacuum to give compound F-3 (356 g, crude, TFA) as a red solid.

LCMS: m/z=236.1 (M+H)+

### General Procedure for Synthesis of Intermediate F

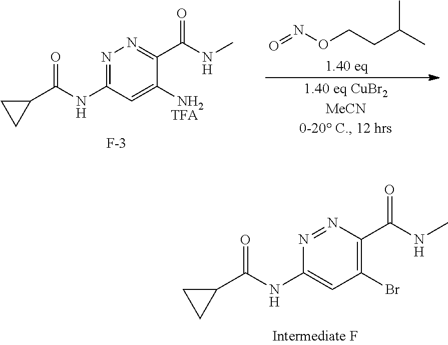

To a solution of compound F-3 (160 g, 192 mmol, 42.0% purity, 1.00 eq, TFA) and isopentyl nitrite (31.6 g, 269 mmol, 36.3 mL, 1.40 eq) in MeCN (800 mL) was added CuBr₂ (60.2 g, 269 mmol, 12.6 mL, 1.40 eq) at 0° C. The mixture was then stirred at 20° C. for 12 hrs until the reaction was determined to be complete by LC/MS analysis after which 10% NH₃·H₂O (3000 mL) was added to the reaction mixture and then extracted with DCM (3000 mL×2). The combined organic phase was washed with 10% NH₃·H₂O (1500 mL×2) and H₂O (2000 mL), dried over anhydrous Na₂SO₄, filtered and concentrated in vacuo. The crude product was triturated with MeOH (400 mL×2) at 20° C. for 3 hrs to provide Intermediate F (26.02 g, 84.8 mmol, 20.1% yield over two steps, combination of 2 batches) as a yellow amorphous solid.

LCMS: m/z=298.9 (M+H)+

¹H NMR (400 MHZ, DMSO-d6): δ 11.8 (s, 1H), 8.79 (d, J=4.4 Hz, 1H), 8.69 (s, 1H), 2.81 (d, J=4.4 Hz, 3H), 2.04-2.10 (m, 1H), 0.88-0.91 (m, 4H).

### Preparation of Intermediate G

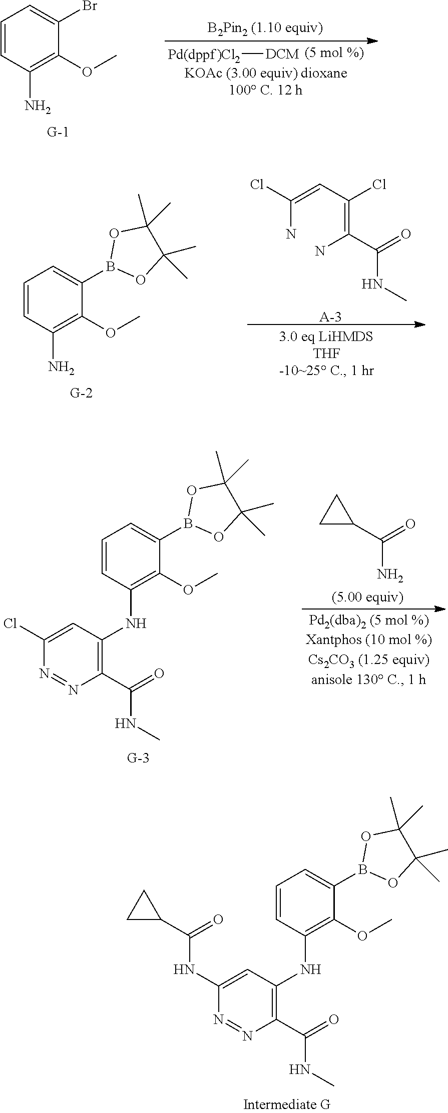

### General Procedure for Synthesis of Compound G-2

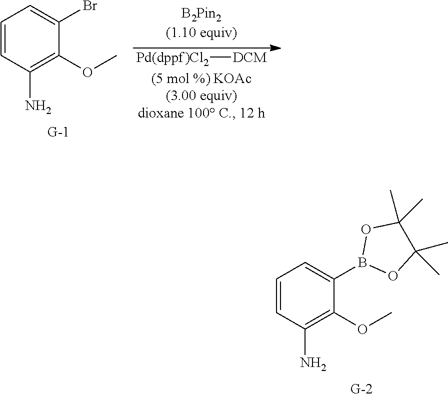

To a solution of compound G-1 (90.0 g, 445 mmol, 1.00 eq) and B₂Pin₂ (124 g, 490 mmol, 1.10 eq) in dioxane (1.80 L) was added KOAc (131 g, 1.34 mol, 3.00 eq) and Pd(dppf)Cl₂·CH₂Cl₂ (18.2 g, 22.3 mmol, 0.05 eq). The mixture was stirred at 100° C. for 12 hrs until the reaction was determined to be complete by LC/MS analysis. The reaction mixture was then poured into H₂O (2.00 L), the aqueous phase was extracted with petroleum ether (2.00 L×4). The combined organic phase was washed with brine (2.00 L×2), dried with anhydrous Na₂SO₄, filtered and concentrated under vacuum to give a crude product. The residue was purified by flash silica gel column (gradient petroleum ether/ethyl acetate) to provide compound G-2 (39.0 g, 157 mmol, 35% yield) as an off-white solid.

LCMS: m/z=249.9 (M+H)+.

¹H NMR (400 MHz, CDCl₃): δ 7.17-7.14 (m, 1H), 6.98-6.88 (m, 2H), 3.95-3.83 (m, 5H), 1.38 (s, 12H).

### General Procedure for Synthesis of Compound G-3

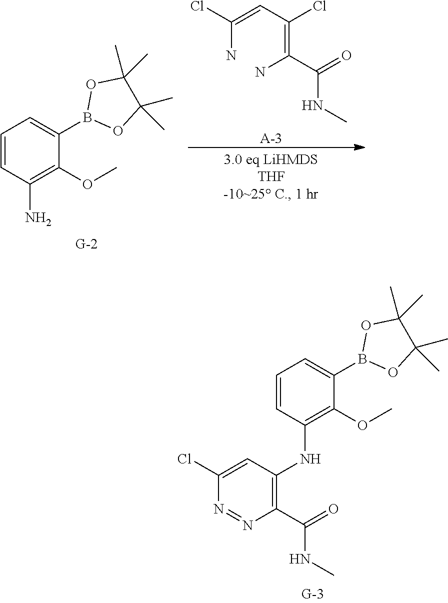 [SMILES: COc1c(N)cccc1B1OC(C)(C)C(C)(C)O1 ; CNC(=O)/C(N)=C(Cl)/C=C(\N)Cl ; "A-3" ; CNC(=O)c1nnc(Cl)cc1Nc1cccc(B2OC(C)(C)C(C)(C)O2)c1OC]

To a solution of compound G-2 (74.0 g, 297 mmol, 1.00 eq), compound 3 (73.4 g, 356 mmol, 1.20 eq) in THF (888 mL) was added drop-wise LiHMDS (1 M, 891 mL, 3.00 eq) at −10 under N₂. The mixture was then stirred at 10° C. for 1 hr after which the reaction was determined to be complete by LC/MS analysis. The reaction mixture was then poured into ice saturated aqueous NH₄Cl (3.00 L), and the aqueous phase was extracted with ethyl acetate (3.00 L). The combined organic phase was washed with brine (3.00 L), dried with anhydrous Na₂SO₄, filtered and concentrated under vacuum. The residue was triturated with petroleum ether/ethyl acetate (10:1 v/v, 1.20 L) at 25° C. for 2 hrs. The mixture was filtered and the cake was collected to provide compound G-3 (120 g, crude) was obtained as brown solid.

LCMS: m/z=419.2 (M+H)+.

¹H NMR (400 MHZ, DMSO-d6) δ 11.0 (s, 1H), 9.36 (d, J=4.8 Hz, 1H), 7.64-7.62 (m, 1H), 7.46-7.44 (m, 1H), 7.21-7.18 (m, 1H), 7.10 (s, 1H), 3.69 (s, 3H), 2.84 (d, J=4.8 Hz, 3H), 1.30 (s, 12H).

### General Procedure for Synthesis of Intermediate G

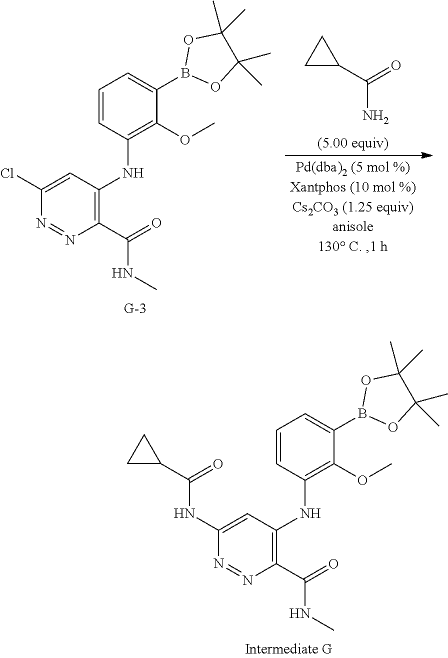

To a solution of compound G-3 (103 g, 246 mmol, 1.00 eq) and cyclopropanecarboxamide (105 g, 1.23 mol, 5.00 eq) in anisole (1.03 L) was added Pd₂(dba)₃ (11.3 g, 12.3 mmol, 0.05 eq), Xantphos (14.2 g, 24.6 mmol, 0.10 eq) and Cs₂CO₃ (100 g, 308 mmol, 1.25 eq). The mixture was stirred at 130° C. for 1 hr until the reaction was determined to be complete by LC/MS analysis. The reaction mixture was cooled to 25° C. and diluted with petroleum (4.00 L). Then the mixture was filtered and the filter cake was collected. The obtained solid was dissolved in dichloromethane/methanol (5:1 v/v, 3.00 L), filtered and the filtrate was washed with brine, the organic phase was dried over anhydrous sodium sulfate, filtered and concentrated under vacuum. The residue was triturated with ethyl acetate/methanol (10:1 v/v, 500 mL) at 25° C. for 2 hrs, filtered and the filter cake was collected via vacuum filtration. The residue was further triturated with ethyl acetate/methanol (5:1 v/v, 600 mL) at 25° C. for 12 hrs, filtered and the filter cake was collected to provide Intermediate G (53.0 g, 109 mmol, 42.7% yield over two steps) as an off-white solid.

LCMS: m/z=468.3 (M+H)+.

¹H NMR (400 MHZ, DMSO-d6): δ 11.3 (s, 1H), 10.9 (s, 1H), 9.13 (d, J=4.8 Hz, 1H), 8.07 (s, 1H), 7.55-7.53 (m, 1H), 7.39-7.37 (m, 1H), 7.20-7.17 (m, 1H), 3.71 (s, 3H), 2.86 (d, J=5.2 Hz, 3H), 2.11-2.05 (m, 1H), 1.32 (s, 12H), 0.83-0.80 (m, 4H).

### Preparation of Intermediate H

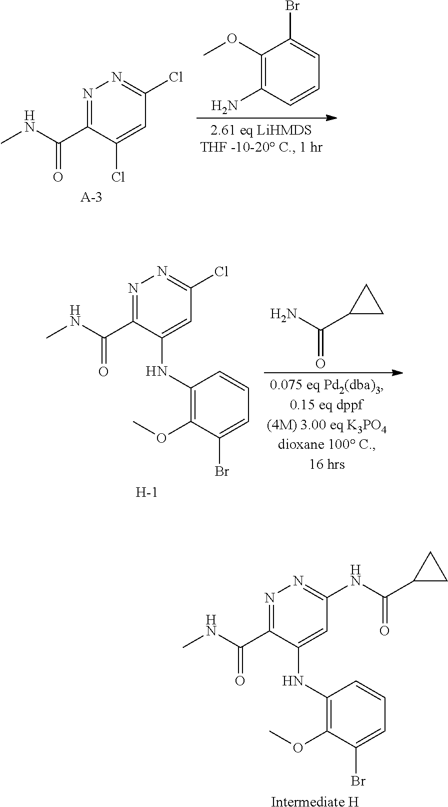

### General Procedure for Preparation of Compound H-1

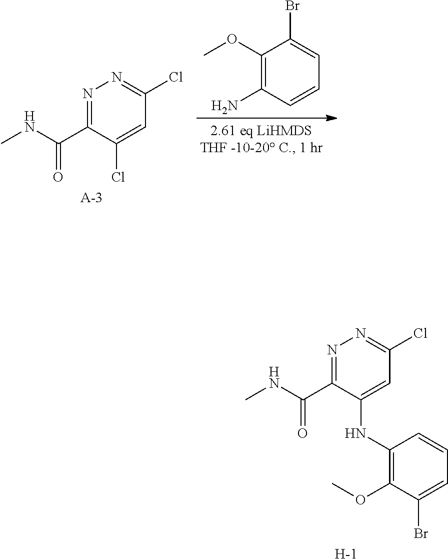

To a solution of compound A-3 (210 g, 958 mmol, 94.0% purity, 1.00 eq) and 2-methoxy-3-bromoaniline (190 g, 940 mmol, 9.81e-1 eq) in THF (1.05 L) was added LiHMDS (1 M, 2.50 L, 2.61 eq) at −10° C. under N₂. The mixture was warmed to 20° C. and stirred at 20° C. for 1 hr until the reaction was determined to be complete by LC/MS analysis. Four reactions were combined for workup/purification. The reaction mixture was poured into ice saturated NH₄Cl solution (8.00 L) and the resulting filter cake was collected. Then the filtrate was then extracted with ethyl acetate (4.00 L×2). The combined organic phase was washed with brine (2.00 L), dried with anhydrous Na₂SO₄, filtered and concentrated under vacuum to give crude product. The combined filter cake and crude product was triturated with petroleum ether/ethyl acetate (5:1 v/v, 2.00 L) at 20° C. for 30 mins, then the mixture was filtered and the filter cake was dried under vacuum to afford compound H-1 (1.30 kg, 3.35 mol, 85% yield) as a yellow solid.

LCMS: m/z=372.9 (M+3H)+.

### General Procedure for Preparation of Intermediate H

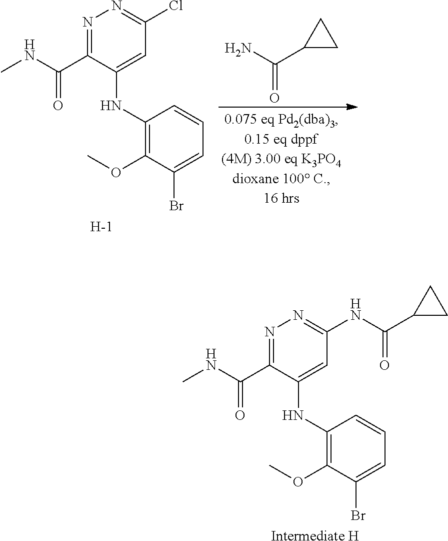

To a solution of compound H-1 (150 g, 403 mmol, 1.00 eq) and cyclopropanecarboxamide (105 g, 1.23 mol, 3.06 eq) in dioxane (1.50 L) was added aqueous K₃PO₄ (4 M, 300 mL, 2.97 eq), Pd₂(dba)₃ (27.7 g, 30.3 mmol, 0.075 eq) and DPPF (33.6 g, 60.6 mmol, 0.15 eq) at 25° C. under N₂ atmosphere. The mixture was de-gassed with N₂ three times and then the mixture was heated to 100° C. and stirred for 16 hrs under N₂ atmosphere until the reaction was determined to be complete by LC/MS analysis. Eight reactions were combined for workup/purification. The resulting mixture was poured into water (16.0 L) and stirred at 25° C. for 0.5 h. The resulting precipitate was isolated by vacuum filtration. The crude product was then triturated with ethyl acetate/methanol (10:1 v/v, 2.00 L) at 20° C. for 2 hrs four times and filtered to provide a light yellow solid (810 g, 1.81 mol) which was triturated with ethyl acetate/methanol (10:1 v/v, 1.65 L) at 20° C. for 16 hrs. The mixture was filtered and washed with ethyl acetate (2.00 L). The cake was dried over vacuum to afford Intermediate H (750 g, 1.70 mol, 52.5% yield) as a light yellow solid.

LCMS: m/z=420.0 (M+H)+.

¹H NMR (400 MHZ, DMSO-d6): δ 11.4 (s, 1H), 11.0 (s, 1H), 9.19 (d, J=4.8 Hz, 1H), 8.13 (s, 1H), 7.54-7.43 (m, 2H), 7.15 (t, J=8.0 Hz, 1H), 3.73 (s, 3H), 2.85 (d, J=4.8 Hz, 3H), 2.14-2.02 (m, 1H), 0.84-0.82 (m, 4H).

### Preparation of Intermediate I

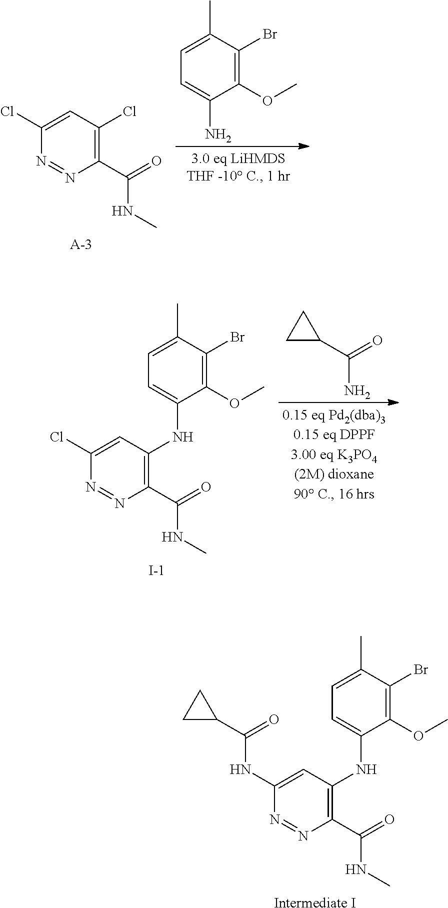

### General Procedure for Preparation of Compound I-1

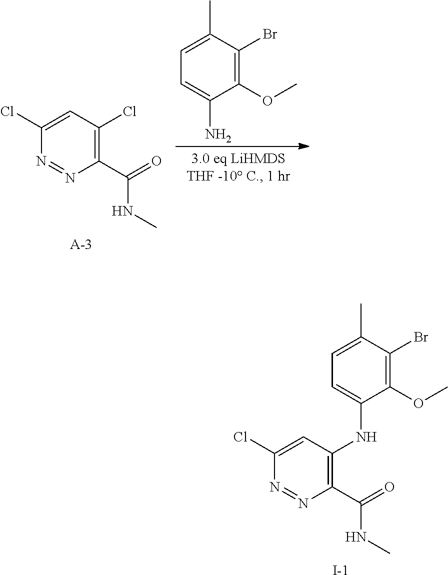 [SMILES: CNC(=O)c1nnc(Cl)cc1Cl ; COc1c(N)ccc(C)c1Br ; "A-3" ; CNC(=O)c1nnc(Cl)cc1Nc1ccc(C)c(Br)c1OC ; [I-]]

To a solution of compound A-3 (33.0 g, 128 mmol, 1.00 eq) and 3-bromo-2-methoxy-4-methylaniline (27.6 g, 128 mmol, 1.00 eq) in THF (600 mL) was added LiHMDS (1 M, 384 mL, 3.00 eq) at −10° C. under N₂. The mixture was then stirred at 20° C. for 1 hr after which the reaction was determined to be complete by LC/MS analysis. The reaction mixture was poured into ice saturated NH₄Cl solution (1.00 L), and the aqueous phase was extracted with ethyl acetate (1.00 L×2). The combined organic phase was washed with brine (2.00 L×2), dried with anhydrous Na₂SO₄, filtered and concentrated under vacuum to give crude residue. This crude residue was purified by trituration with petroleum ether/ethyl acetate (5:1 v/v, 200 mL) at 20° C. for 30 mins. The solid was filtered and the filter cake was dried under vacuum to provide compound I-1 (42.0 g, 105 mmol, 82.3% yield,) as a brown solid.

LCMS: m/z=387.0 (M+H)+.

¹H NMR (400 MHZ, DMSO-d6): δ 11.02 (s, 1H), 9.40-9.39 (m, 1H), 7.45 (d, J=8.4 Hz, 1H), 7.22 (d, J=8.4 Hz, 1H), 7.10 (s, 1H), 3.70 (s, 3H), 2.86 (d, J=4.8 Hz, 3H), 2.37 (s, 3H).

### General Procedure for Preparation of Intermediate I

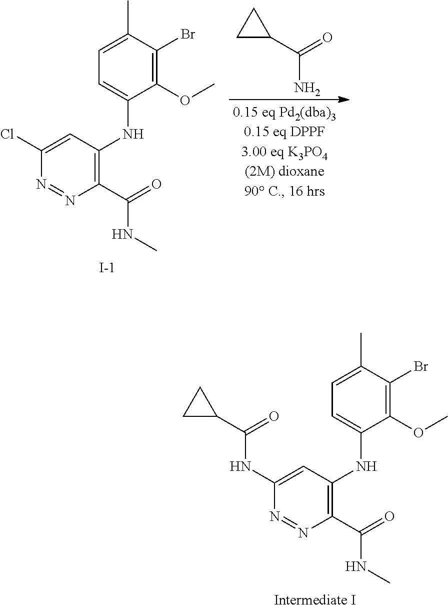 [SMILES: CNC(=O)c1nnc(Cl)cc1Nc1ccc(C)c(Br)c1OC ; NC(=O)C1CC1 ; [I-] ; CNC(=O)c1nnc(NC(=O)C2CC2)cc1Nc1ccc(C)c(Br)c1OC ; "Intermediate I"]

To solution of compound I-1 (25.0 g, 64.8 mmol, 1.00 eq) and cyclopropylcarboxamide (16.5 g, 194 mmol, 3.00 eq) in dioxane (750 mL) was added K₃PO₄ (2 M, 97.2 mL, 3.00 eq), Pd₂(dba)₃ (8.90 g, 9.72 mmol, 0.15 eq) and DPPF (5.39 g, 9.72 mmol, 0.15 eq) at 25° C. The mixture was then degassed with N₂ three times, then the mixture was heated to 90° C. and stirred at 90° C. for 16 hrs under N₂ atmosphere until the reaction was determined to be complete by LC/MS analysis. The mixture was filtered and filtrate cake was washed with ethyl acetate (500 mL×2), then the filtrate was diluted with ethyl acetate (2.00 L) and washed with brine (2.00 L×3), dried with anhydrous Na₂SO₄, filtered and concentrated in vacuum to give a crude product. The yellow solid was purified by trituration with ethyl acetate (200 mL) at 20° C. for 3 hrs, then filtered to give a Intermediate I (10.4 g, 22.8 mmol, 35.2% yield) as a yellow solid.

LCMS: m/z=436.0 (M+H)+.

¹H NMR (400 MHZ, DMSO-d6): δ 11.33 (s, 1H), 10.86 (s, 1H), 9.18-9.15 (m, 1H), 8.04 (s, 1H), 7.36 (d, J=8.0 Hz, 1H), 7.22 (d, J=8.4 Hz, 1H), 3.69 (s, 3H), 2.85 (d, J=5.2 Hz, 3H), 2.36 (s, 3H), 2.10-2.05 (m, 1H), 0.83-0.79 (m, 4H).

### Preparation of Intermediate J

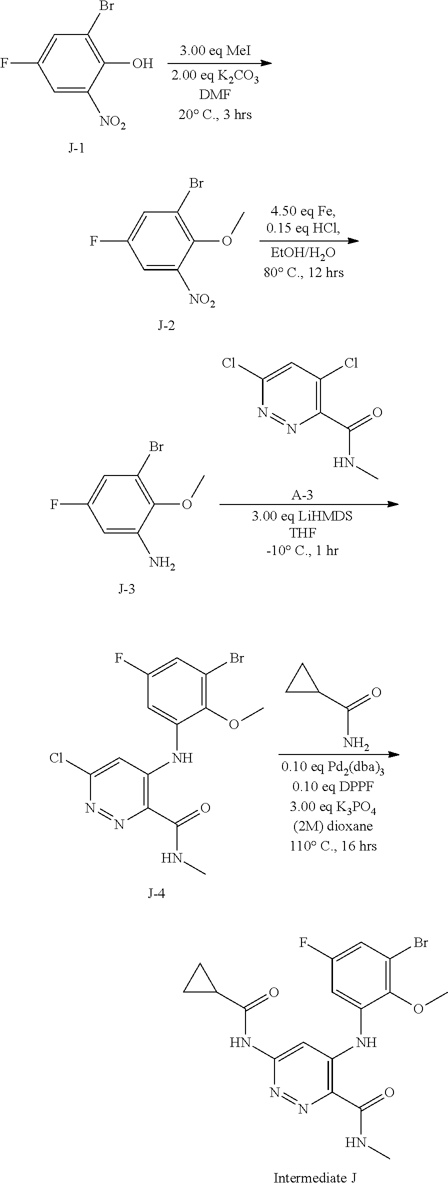

### General Procedure for Synthesis of Compound J-2

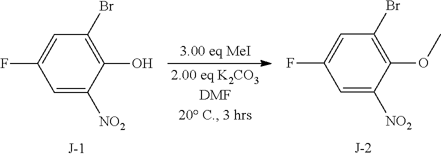

To a mixture of compound J-1 (100 g, 424 mmol, 1.00 eq) in DMF (1.00 L) was added K₂CO₃ (117 g, 847 mmol, 2.00 eq), MeI (180 g, 1.27 mol, 79.1 mL, 3.00 eq), and the mixture was stirred at 20° C. for 12 hrs until the reaction was determined to be complete by HPLC analysis. The mixture was poured into H₂O (1.50 L) and extracted with ethyl acetate (1.00 L×2), the combined organic layers were washed with brine (1.00 L×4), and concentrated under reduced pressure to province compound J-2 (100 g, 400 mmol) as a yellow solid.

¹H NMR (400 MHz, DMSO-d6): δ 8.12-8.09 (m, 1H), 8.04-8.02 (m, 1H), 3.90 (s, 3H)

### General Procedure for Synthesis of Compound J-3

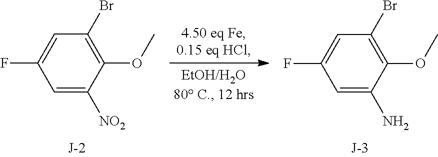

To a mixture of compound J-2 (100 g, 400 mmol, 1.00 eq) in EtOH (1.00 L) was added H₂O (1.00 L), Fe (100 g, 1.79 mol, 4.48 eq) at 20° C., then the mixture was dropwise added HCl (12.0 M, 5.00 mL, 0.15 eq) at 80° C. The mixture was then stirred at 80° C. for 12 hrs until the reaction was determined to be complete by LC/MS analysis. The mixture was filtered and the filtrate was concentrated to remove EtOH, after which H₂O (300 mL) was added, followed by saturated NaHCO₃ solution (300 mL) and extracted with ethyl acetate (500 mL×2). The combined organic layers were washed with brine (500 mL×3), concentrated under reduced pressure to give compound J-3 (80.0 g, 364 mmol) as a brown oil.

¹H NMR (400 MHZ, DMSO-d6): δ 6.55-6.52 (m, 1H), 6.47-6.44 (m, 1H), 5.59 (s, 2H), 3.63 (s, 3H)

### Genera Procedure for Synthesis of Compound J-4

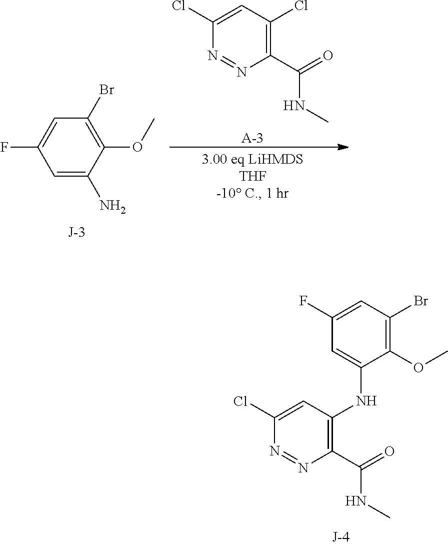 [SMILES: COc1c(N)cc(F)cc1Br ; CNC(=O)c1nnc(Cl)cc1Cl ; "A-3" ; CNC(=O)c1nnc(Cl)cc1Nc1cc(F)cc(Br)c1OC]

To a solution of compound J-3 (40.0 g, 194 mmol, 1.00 eq) and compound 3 (42.7 g, 194 mmol, 1.00 eq) in THF (800 mL) was added LiHMDS (1 M, 582 mL, 3.00 eq) at −10° C. under N₂. The mixture was stirred at 0° C. for 1 hr until the reaction was determined to be complete by LC/MS analysis. The reaction mixture was poured into saturated NH₄Cl solution (2.00 L), and the aqueous phase was extracted with ethyl acetate (2.00 L×2). The combined organic phase was washed with brine (2.00 L×2), dried with anhydrous Na₂SO₄, filtered and concentrated under vacuum to give a crude product. The residue was purified by triturated with petroleum ether/ethyl acetate (1:1 v/v, 200 mL) at 20° C. for 30 mins, and then filtered to provide compound J-4 (60.0 g, 154 mmol) as a yellow solid.

¹H NMR (400 MHZ, DMSO-d6): δ 11.3 (s, 1H), 9.45-9.43 (m, 1H), 7.58-7.55 (m, 1H), 7.46-7.43 (m, 2H), 3.72 (s, 3H), 2.86 (d, J=4.8 Hz, 3H).

### General Procedure for Preparation of Intermediate J

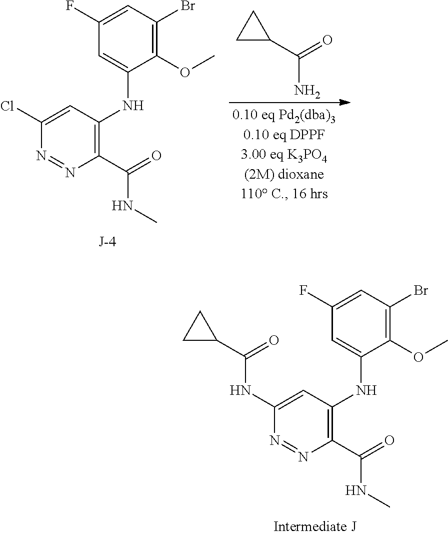

To solution of compound J-4 (50.0 g, 128 mmol, 1.00 eq) and cyclopropylcarboxamide (21.8 g, 256 mmol, 2.00 eq) in dioxane (1.50 L) was added K₃PO₄ (2 M, 192 mL, 3.00 eq), Pd₂(dba)₃ (17.6 g, 19.2 mmol, 0.15 eq) and DPPF (10.6 g, 19.2 mmol, 0.15 eq). The mixture was then stirred at 110° C. for 12 hrs under N₂ atmosphere after which the reaction was determined to be complete by LC/MS analysis. The reaction mixture was then poured into H₂O (2.00 L), and the aqueous phase was extracted with ethyl acetate (1.00 L×3). The combined organic phase was washed with brine (2.00 L×2), dried with anhydrous Na₂SO₄, filtered and concentrated under vacuum to give a crude product. The residue was purified by trituration with petroleum ether:ethyl acetate (3:1 v/v, 200 mL) at 20° C. for 30 mins, and filtered to give a yellow solid. This residue was further purified by trituration with dichloromethane:ethyl acetate (1:1 v/v, 100 mL) at 20° C. for 30 mins, and filtered to provide Intermediate J as a yellow solid (23.0 g, 50.9 mmol, 40.0% yield).

LCMS: m/z=439.9 (M+H)+.

¹H NMR (400 MHZ, DMSO-d6): δ 11.4 (s, 1H), 11.2 (s, 1H), 9.23-9.21 (m, 1H), 8.22 (s, 1H), 7.42-7.36 (m, 2H), 3.73 (s, 3H), 2.85 (d, J=4.8 Hz, 3H), 2.10-2.07 (m, 1H), 0.85 (d, J=5.6 Hz, 4H).

19F NMR (400 MHZ, DMSO-d6): δ 114.504 ppm

### Preparation of Intermediate K

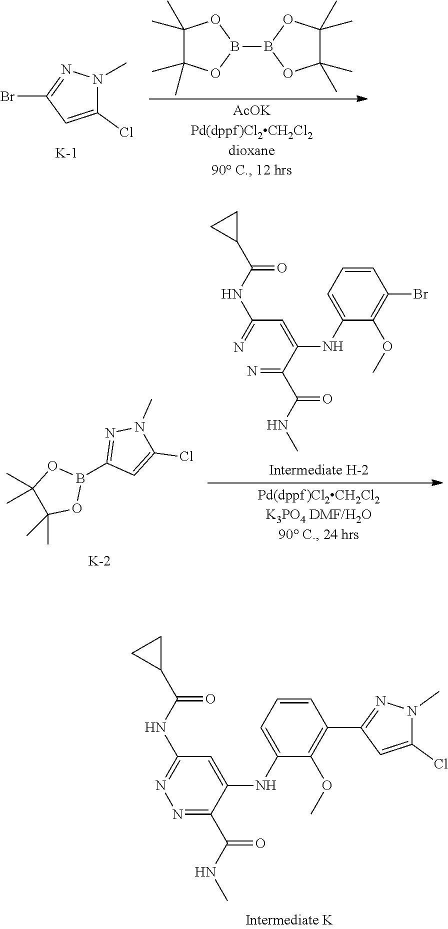

### General Procedure for Synthesis of Compound K-2

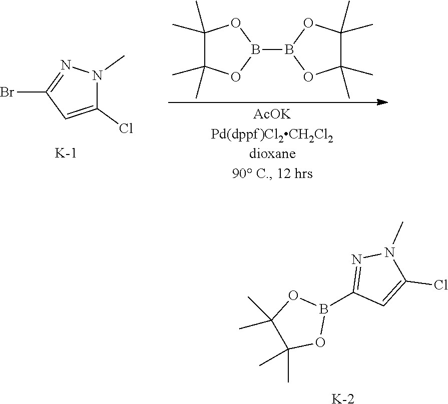

To a solution of compound K-1 (32.0 g, 163 mmol, 1.00 eq) in dioxane (320 mL) was added bi(pinacolato)diboron (62.3 g, 245 mmol, 1.50 eq) and Pd(dppf)Cl₂·CH₂Cl₂ (6.69 g, 8.19 mmol, 0.05 eq) and AcOK (40.1 g, 409 mmol, 2.50 eq). The mixture was then stirred at 90° C. for 12 hrs under N₂ until the reaction was determined to be complete by LC/MS analysis. The reaction mixture was filtered through celite and the filtrate was concentrated under vacuum. The crude product was purified by flash silica gel chromatography (gradient petroleum ether/ethyl acetate) to obtain compound K-2 (44.0 g, 108 mmol, 66.4% yield) as a yellow gum.

LCMS: m/z=242.9 (M+H)+

1H NMR: (400 MHz, CDCl₃): δ 6.57 (s, 1H), 3.89 (s, 3H), 1.34 (s, 12H).

### General Procedure for Intermediate K

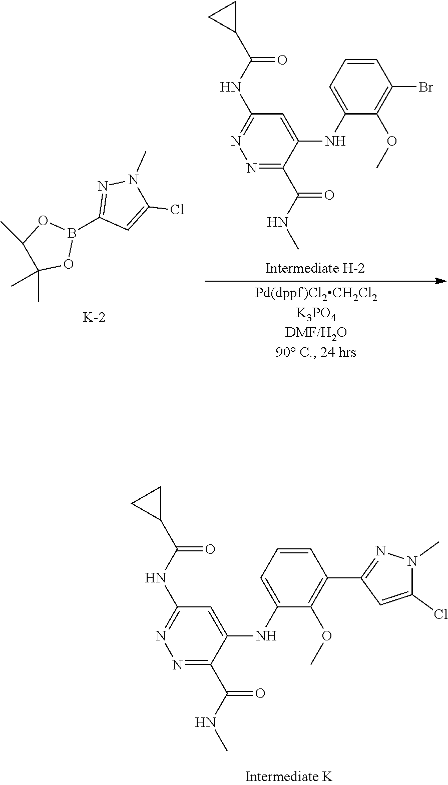

To a solution of compound K-2 (15.0 g, 35.6 mmol, 1.00 eq) and Intermediate H (2.50 eq) in DMF (300 mL) and H₂O (60.0 mL) was added Pd(dppf)Cl₂·CH₂Cl₂ (4.37 g, 5.35 mmol, 0.15 eq) and K₃PO₄ (15.1 g, 71.3 mmol, 2.00 eq). The mixture was then stirred at 90° C. for 24 hrs under N₂ until the reaction was determined to be complete by LC/MS analysis. The reaction mixture was poured into water (1.20 L), extracted with ethyl acetate (200 mL×4), and the combined organic layers washed with brine (500 mL*2), dried over Na₂SO₄, filtered and concentrated under vacuum. The crude product was purified by flash silica gel chromatography (gradient petroleum ether:/ethyl acetate 2% methanol/dichloromethane) to obtain Intermediate K (10.76 g, 22.7 mmol, 64% yield) as a yellow solid.

LCMS: m/z=456.0 (M+H)+

1H NMR: (400 MHz, DMSO-d6): δ 11.3 (s, 1H), 11.0 (s, 1H), 9.17 (d, J=5.2 Hz, 1H), 8.14 (s, 1H), 7.66 (dd, J=7.6, 1.2 Hz, 1H), 7.43 (d, J=6.8 Hz, 1H), 7.25 (t, J=7.8 Hz, 1H), 6.82 (s, 1H), 3.88 (s, 3H), 3.61 (s, 3H), 2.86 (d, J=4.8 Hz, 3H), 2.10-2.04 (m, 1H), 0.82 (t, J=3.8 Hz, 4H).

### Preparation of Intermediate L

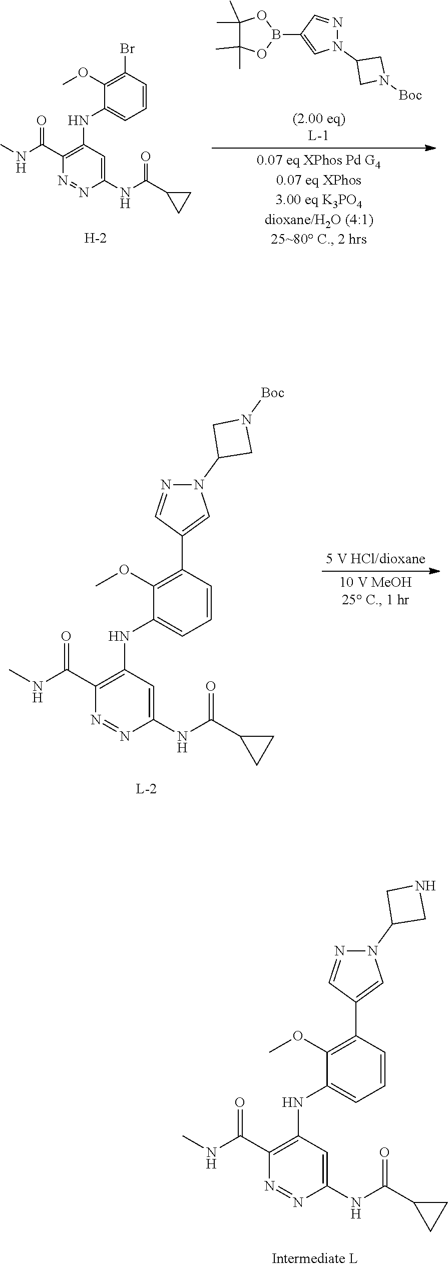

### General Procedure for Synthesis of Compound L-2

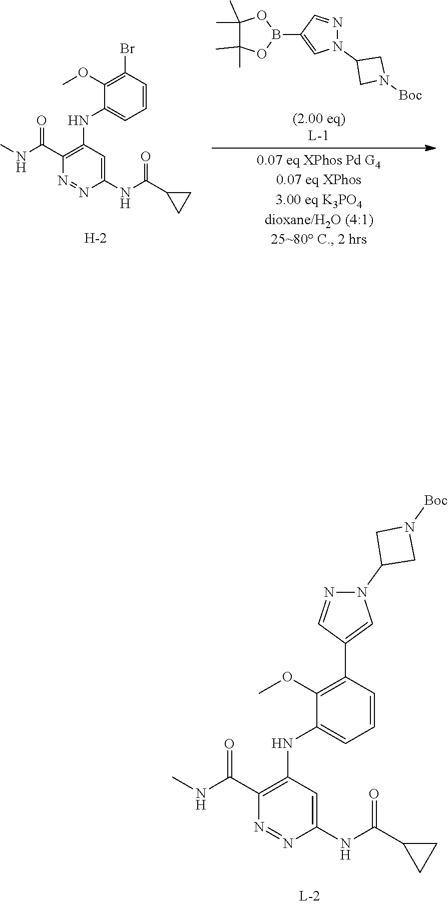

To a solution of compound H-2 (45.0 g, 107 mmol, 1.00 eq) and compound L-1 (74.8 g, 214 mmol, 2.00 eq) in dioxane (900 mL) and H₂O (225 mL) was added XPhos (3.57 g, 7.50 mmol, 0.07 eq), K₃PO₄ (68.2 g, 321 mmol, 3.00 eq) and Xphos Pd G4 (6.45 g, 7.50 mmol, 0.07 eq) at 25° C. under N₂, and the mixture was degassed and purged with N₂ for 3 times. Then the mixture was then heated to 80° C. and stirred at 80° C. for 2 hrs until the reaction was determined to be complete by LC/MS analysis. The reaction mixture was then cooled to 25° C., poured into H₂O (4.00 L) and extracted with dichloromethane (1.00 L×3). The combined organic layers were washed with brine (1.00 L), dried over Na₂SO₄, filtered and concentrated under reduced pressure to give a residue. The residue was purified by flash silica column chromatography (gradient petroleum ether/ethyl acetate) and concentrated under reduced pressure to give compound L-2 (45.0 g, 76.7 mmol, 72% yield) as a yellow solid.

LCMS: m/z=563.2 (M+H)+

¹H NMR (400 MHZ, DMSO-d6): δ 11.31 (s, 1H), 10.95 (s, 1H), 9.16 (d, J=4.8 Hz, 1H), 8.34 (s, 1H), 8.14 (s, 1H), 8.08 (s, 1H), 7.48-7.46 (m, 1H), 7.30-7.28 (d, J=8.0 Hz, 1H), 7.21-7.17 (m, 1H), 5.32-5.25 (m, 1H), 4.32-4.29 (m, 2H), 4.18 (s, 2H), 3.59 (s, 3H), 2.86 (d, J=4.4 Hz, 3H), 2.09-2.02 (m, 1H), 1.41 (s, 9H), 0.83-0.80 (m, 4H)

### General Procedure for Intermediate L

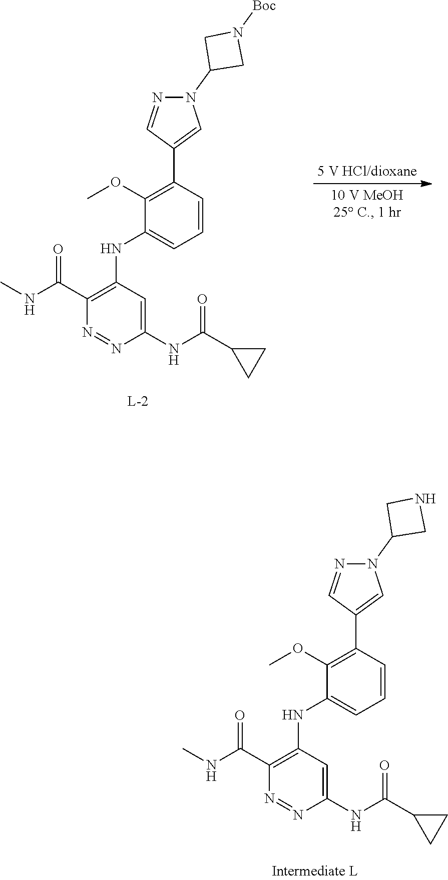 [SMILES: CNC(=O)c1nnc(NC(=O)C2CC2)cc1Nc1cccc(-c2cnn(C3CN(C(=O)OC(C)(C)C)C3)c2)c1OC ; CNC(=O)c1nnc(NC(=O)C2CC2)cc1Nc1cccc(-c2cnn(C3CNC3)c2)c1OC]

To a solution of compound L-2 (70.0 g, 124 mmol, 1.00 eq) in MeOH (700 mL) was added HCl/dioxane (4 M, 350 mL, 11.3 eq), and the mixture was stirred at 25° C. for 1 hr after which the reaction was determined to be complete by LC/MS analysis. The reaction mixture was then concentrated under reduced pressure to give crude product 70.0 g (HCl salt). The HCl salt was dissolved in H₂O (4.00 L) and the mixture was adjusted the pH to 8 with saturated NaHCO₃ solution and the resulting solid was filtered and the filter cake was dried under reduced pressure to provide crude residue (39.0 g). The filtrate was then extracted with dichloromethane/methanol (10:1 v/v, 1.00 L×3). The combined organic layers were washed with brine (1.00 L×2), dried over Na₂SO₄, filtered and concentrated to give additional solid (30.0 g). The combined solids were triturated with dichloromethane/ethyl acetate (3.5:1 v/v, (200 mL) at 25° C. for 1 hr. The solid was filtered and dried over vacuum to provide Intermediate L (56.8 g, 116 mmol, 65% yield) as a yellow solid.

LCMS: m/z=463.2 (M+H)+

¹H NMR (400 MHZ, DMSO-d6): δ 11.30 (s, 1H), 10.96 (s, 1H), 9.16-9.13 (m, 1H), 8.32 (s, 1H), 8.14 (s, 1H), 8.08 (s, 1H), 7.47-7.45 (m, 1H), 7.30 (d, J=8.0 Hz, 1H), 7.21-7.17 (m, 1H), 5.26-5.22 (m, 1H), 3.98-3.94 (m, 2H), 3.81-3.73 (m, 2H), 3.58 (s, 3H), 2.86 (d, J-4.8 Hz, 3H), 2.08-2.06 (m, 1H), 0.82-0.80 (m, 4H).

### Preparation of Intermediate M

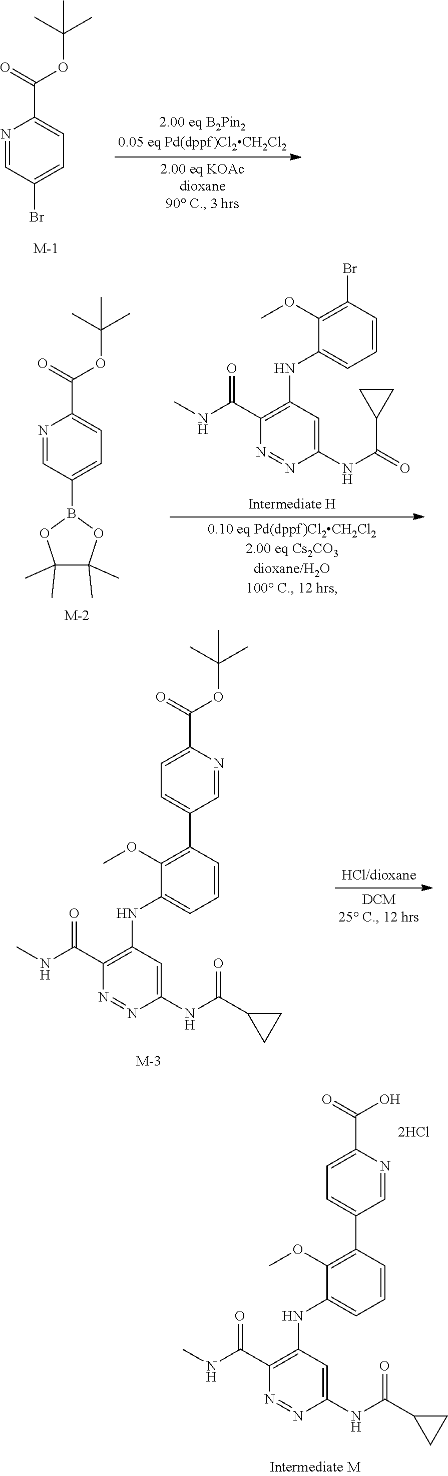

### General Procedure for Synthesis of Compound M-2

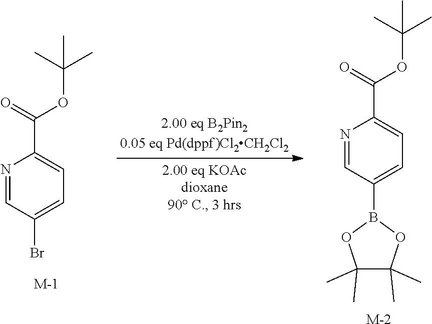

To a solution of compound M-1 (70.0 g, 271 mmol, 1.00 eq) and B₂Pin₂ (137 g, 542 mmol, 2.00 eq) in dioxane (700 mL) was added KOAc (53.2 g, 542 mmol, 2.00 eq) and Pd(dppf)Cl₂·CH₂Cl₂ (11.0 g, 13.5 mmol, 0.05 eq) at 25° C. The mixture was the heated to 90° C. and stirred at 90° C. for 4 hrs under N₂ until the reaction was determined to be complete by LC/MS analysis. The reaction mixture was poured into H₂O (2.00 L), and the aqueous phase was extracted with ethyl acetate (2.00 L×2). The combined organic phase was washed with brine (2.00 L×2), dried with anhydrous Na₂SO₄, filtered and concentrated under vacuum to give a crude product. The residue was then triturated with petroleum ether (500 mL) at 20° C. for 2 hrs, filtered and dried under vacuum at 50° C. to provide compound M-2 (72.0 g, 235 mmol, 87.0% yield) as a yellow solid.

LCMS: m/z=168.1 (M-137)+.

¹H NMR (400 MHZ, DMSO-d6): δ 8.85 (d, J=0.4 Hz, 1H), 8.17-8.15 (m, 1H), 7.98 (d, J=8.0 Hz, 1H), 1.56 (s, 9H), 1.33 (s, 12H).

### General Procedure for Synthesis of Compound M-3

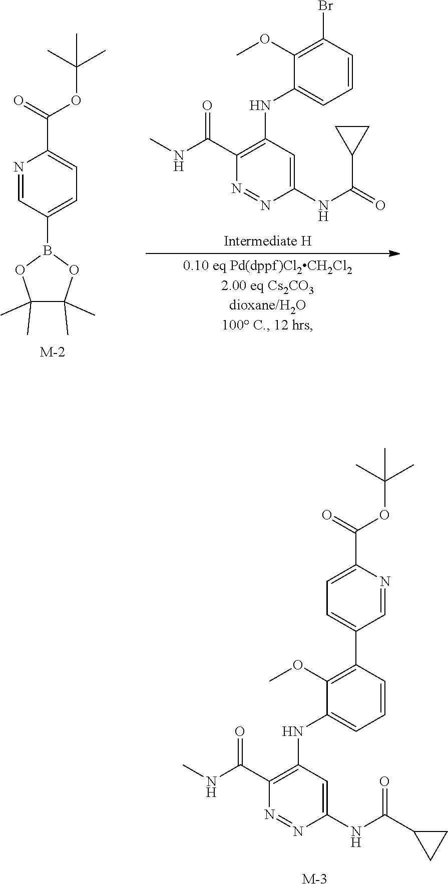

To a solution of Intermediate H (92.0 g, 218 mmol, 1.00 eq) and compound M-2 (80.1 g, 262 mmol, 1.20 eq) in H₂O (184 mL) and dioxane (920 mL) was added Pd(dppf)Cl₂·CH₂Cl₂ (17.8 g, 21.8 mmol, 0.10 eq) and Cs₂CO₃ (142 g, 437 mmol, 2.00 eq) at 25° C. The mixture was then heated to 100° C. and stirred at 100° C. for 2 hrs under N₂ until the reaction was determined to be complete by LC/MS analysis. The reaction mixture was poured into H₂O (2.00 L), the aqueous phase was extracted with ethyl acetate (2.00 L×2). The combined organic phase was washed with brine (2.00 L×2), dried with anhydrous Na₂SO₄, filtered and concentrated under vacuum to give a crude product. The crude product was purified by flash silica gel chromatography (gradient dichloromethane/methanol) to provide compound M-3 (95.0 g, 172 mmol, 78.7% yield) as a yellow solid.

LCMS: m/z=519.1 (M+H)+.

¹H NMR (400 MHZ, DMSO-d6): δ 11.36 (s, 1H), 11.01 (s, 1H), 9.20-9.17 (m, 1H), 8.89 (d, J=1.2 Hz, 1H), 8.20 (s, 1H), 8.18-8.16 (m, 1H), 8.10-8.08 (m, 1H), 7.55 (d, J=6.0 Hz, 1H), 7.36-7.32 (m, 2H), 3.39 (s, 3H), 2.87 (d, J=4.8 Hz, 3H), 2.12-2.06 (m, 1H), 1.59 (s, 9H), 0.85-0.83 (m, 4H).

### General Procedure for Synthesis of Intermediate M

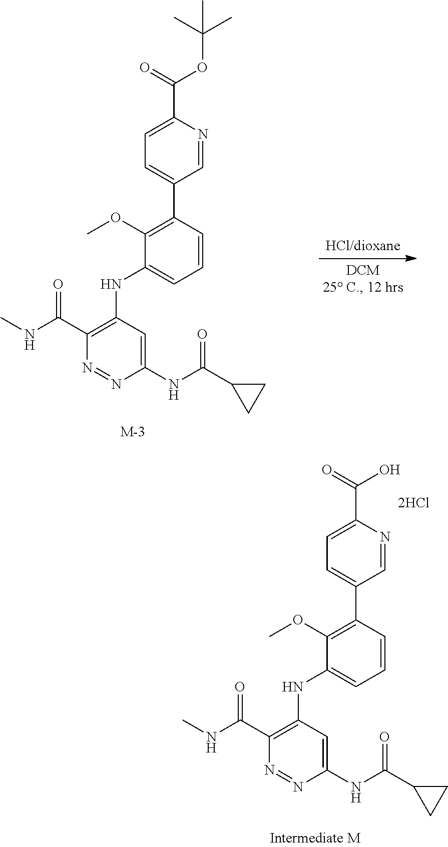 [SMILES: CNC(=O)c1nnc(NC(=O)C2CC2)cc1Nc1cccc(-c2ccc(C(=O)OC(C)(C)C)nc2)c1OC ; "M-3" ; CNC(=O)c1nnc(NC(=O)C2CC2)cc1Nc1cccc(-c2ccc(C(=O)O)nc2)c1OC]

To a solution of compound M-3 (95.0 g, 183 mmol, 1.00 eq) in DCM (950 mL) was added HCl/dioxane (4 M, 950 mL, 20.7 eq) at 25° C., and the mixture was stirred at 25° C. for 12 hrs until the reaction mixture was concentrated under vacuum to give a crude product. The residue was purified by trituration with MeCN (1.00 L) at 20° C. for 12 hrs, filtered and dried under vacuum at 50° C. to provide Intermediate M (83.0 g, 151 mmol, 82.9% yield, dihydrochloride salt) as a yellow solid.

LCMS: m/z=463.1 (M+H)+.

¹H NMR (400 MHZ, DMSO-d6): δ 11.85 (s, 1H), 11.22 (s, 1H), 9.19-9.18 (m, 1H), 8.92 (d, J=1.2 Hz, 1H), 8.28-8.26 (m, 1H), 8.18 (d, J=8.0 Hz, 1H), 8.03 (s, 1H), 7.58-7.56 (m, 1H), 7.41-7.37 (m, 2H), 3.40 (s, 3H), 2.86 (d, J=4.8 Hz, 3H), 2.10-2.04 (m, 1H), 0.92-0.85 (m, 4H).

### Preparation of Intermediate N

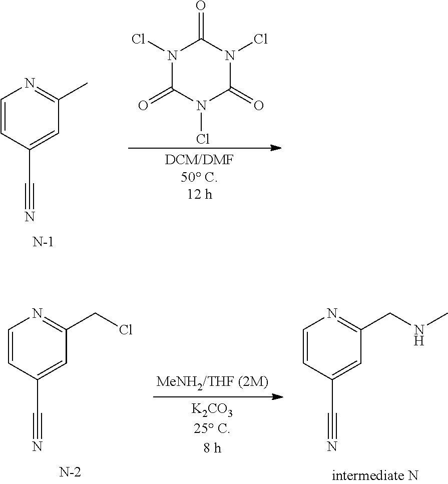

### General Procedure for Synthesis of Compound N-2

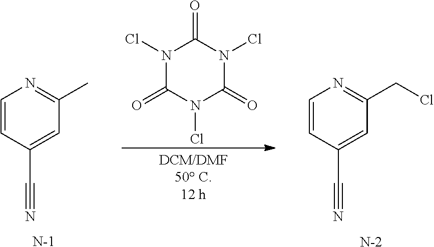

To a solution of compound N-1 (50.0 g, 423 mmol, 1.00 eq) in dichloromethane (1.00 L) was added DMF (3.09 g, 42.3 mmol, 3.26 mL, 0.100 eq) at 25° C., then trichloroisocyanuric acid (128 g, 550 mmol, 1.30 eq) was added at 25° C. The mixture was then heated to 50° C. and stirred at 50° C. for 12 hrs until the reaction was determined to be complete by LC/MS analysis. The mixture was then filtered and the filter cake was washed with dichloromethane (300 mL×3). The filtrate was poured into 10% Na₂CO₃ aqueous solution (1.00 L) slowly. The mixture was separated, the organic layer was washed with brine (1.00 L), dried over Na₂SO₄, filtered and concentrated. The residue was purified by flash silica gel column chromatography (gradient petroleum ether/ethyl acetate) to provide compound N-2 (64.6 g, crude) was obtained as brown liquid.

¹H NMR (400 MHZ, DMSO-d6): δ 8.83 (dd, J=5.2, 0.8 Hz, 1H), 8.03 (d, J=1.2 Hz, 1H), 7.85 (dd, J=4.8, 1.6 Hz, 1H), 4.84 (s, 2H).

### General Procedure for Preparation of Intermediate N

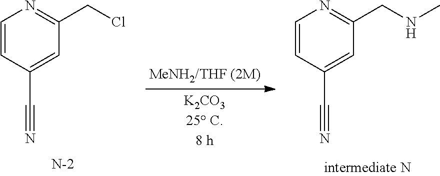

To a solution of compound N-2 (74.5 g, 488 mmol, 1.00 eq) in MeNH₂/THF (2 M, 2.44 L, 10.0 eq) was added K₂CO₃ (202 g, 1.46 mol, 3.00 eq). Then the mixture was stirred at 25° C. for 8 hrs until the reaction was determined to be complete by LC/MS analysis. The mixture was diluted with water (1.00 L), extracted with ethyl acetate (1.00 L×2) and dichloromethane/MeOH (1.10 L×2), the combined organic layers were dried with Na₂SO₄, filtered and concentrated. The crude product was purified by reverse-phase HPLC (0.1% NH₃·H₂O), the eluent was concentrated to remove most of MeCN at 50° C., then extracted with dichloromethane/MeOH (5:1 v/v, 1.92 L×5). The combined organic layers were dried with Na₂SO₄ and filtered. The filtrate was concentrated under vacuum to provide Intermediate N (52.0 g, 346 mmol, 71% yield) as a yellow oil.

LCMS (ES+): m/z=148.1 (M+H)+

¹H NMR (400 MHZ, MeOD): δ 8.75 (d, J=5.2 Hz, 1H), 7.74 (s, 1H), 7.61 (dd, J=5.2, 1.2 Hz, 1H), 3.90 (s, 2H), 2.42 (s, 3H).

The examples in Table 1 were prepared according to the following procedures:

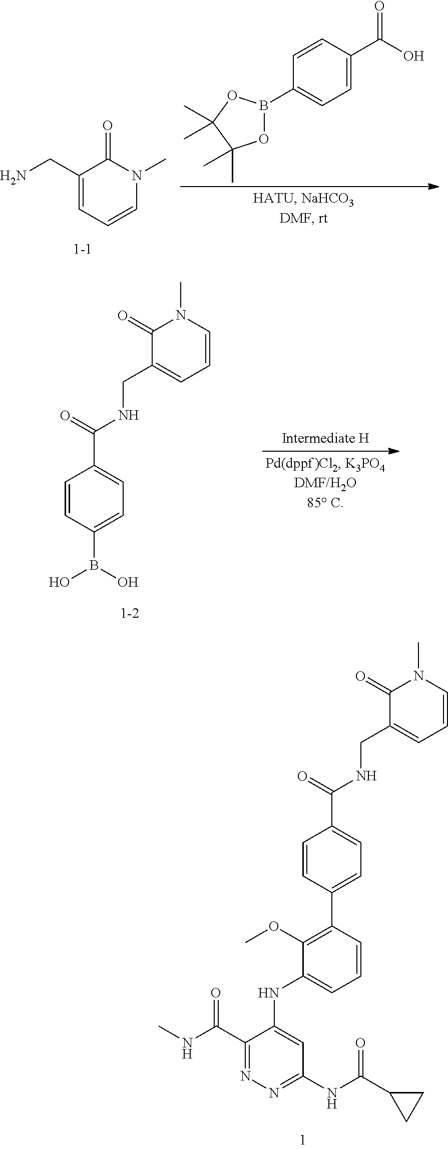

Step 1: To a stirred solution of 3-(aminomethyl)-1-methylpyridin-2 (1H)-one (compound 1-1, 300 mg, 1.0 equiv, 2.17 mmol), sodium hydrogen carbonate (547 mg, 3.0 equiv, 6.51 mmol) and HATU (1.24 g, 1.5 equiv, 3.26 mmol) in DMF (5 mL) was added 4-(4,4,5,5-tetramethyl-1,3,2-dioxaborolan-2-yl)benzoic acid (539 mg, 1.0 equiv, 2.17 mmol) at room temperature. The resulting mixture was then stirred at 25° C. for 1 hour until the reaction was determined to be complete by LC/MS analysis. The reaction was quenched with water and extracted with ethyl acetate. The organic layer was washed with brine, dried over Na₂SO₄ and evaporated to provide compound 1-2 (400 mg, 1.40 mmol, 64%) as a white solid.

LC/MS m/z (ES+): 287.05 [M+H]+

Step 2: A round bottomed flask was charged with Intermediate H (120 mg, 1.0 equiv, 286 μmol), compound 1-2 (123 mg, 1.5 equiv, 428 μmol) (2.8 g, 4 eq), tripotassium phosphate (121 mg, 2.0 equiv, 571 μmol), Pd(dppf)Cl₂·CH₂Cl₂ (10 mol %) and a stirbar after which DMF/water (3:1 v/v, 4 mL) was added. The solution was stirred at 85° C. for 1 hour after which the reaction was determined to be complete by LC/MS analysis. The reaction was quenched with water and extracted with ethyl acetate. The organic layer was washed with brine, dried over Na₂SO₄ and evaporated. The resulting crude material was purified using prep-HPLC (column: XBridge Shield RP18 OBD Column, 30×150 mm, 5 μm, gradient of water (10 mmol/L NH₄HCO3+0.1% NH₃·H₂O)/MeCN) to provide compound 1 (77.6 mg, 133 μmol, 47%) as a white amorphous solid.

LC/MS m/z (ES+): 582.15 [M+H]+

¹H NMR (400 MHZ, DMSO-d6): 11.34 (s, 1H), 11.01 (s, 1H), 9.17 (q, J=4.7 Hz, 1H), 8.91 (t, J=5.8 Hz, 1H), 8.21 (s, 1H), 8.04-7.97 (m, 2H), 7.73-7.66 (m, 2H), 7.63 (dd, J=6.7, 2.0 Hz, 1H), 7.50 (dd, J=7.9, 1.7 Hz, 1H), 7.35-7.21 (m, 3H), 6.22 (t, J=6.8 Hz, 1H), 4.29 (d, J=5.7 Hz, 2H), 3.48 (s, 3H), 3.37 (s, 3H), 2.86 (d, J=4.8 Hz, 3H), 2.10 (p, J=6.3 Hz, 1H), 0.89-0.82 (m, 4H).

### Example 2

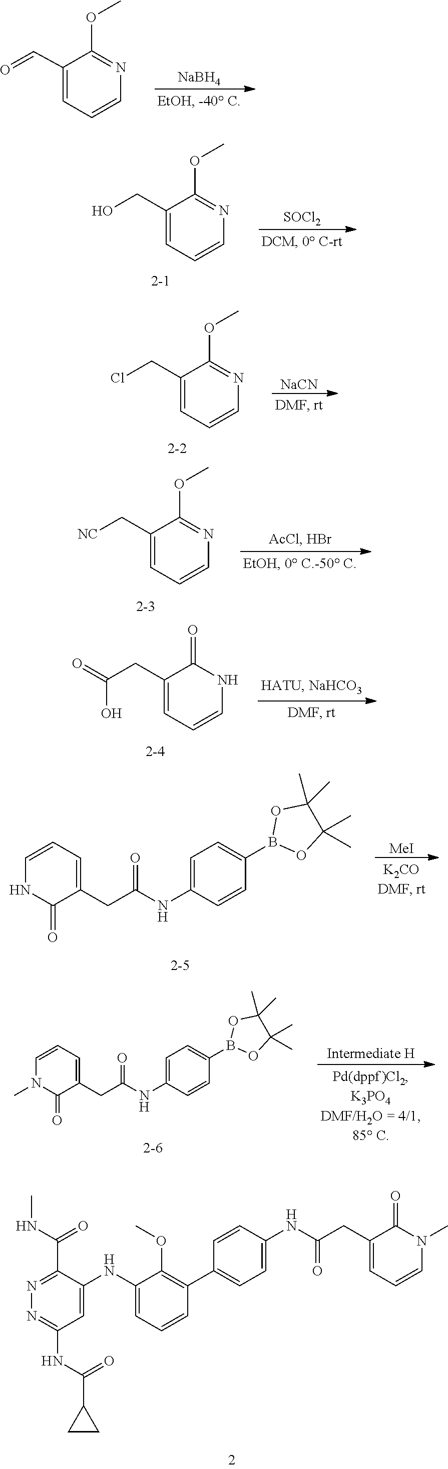

Step 1: To a stirred solution of 2-methoxynicotinaldehyde (5 g, 1.0 equiv, 0.04 mol) in EtOH (50 mL) was added NaBH₄ (3.0 g, 2.0 equiv, 0.07 mol) at −40° C. The resulting mixture was stirred at −40° C. for 2 h after which the reaction was quenched with water and extracted with ethyl acetate. The organic layer was washed with brine, dried over Na₂SO₄ and evaporated to provide compound 2-1 (4.4 g, 32 mmol, 90%) as a white solid.

LC/MS m/z (ES+): 140.10 [M+H]+

Step 2: To a stirred solution of compound 2-1 (4 g, 1.0 equiv, 0.03 mol) in DCM (40 mL) was added SOCl₂ (5 g, 3 mL, 1.5 equiv, 0.04 mol) at 0° C. The resulting mixture was stirred at room temperature for 1 h. The reaction was quenched with aqueous NaHCO₃ and extracted with ethyl acetate. The organic layer was washed with brine, dried over Na₂SO₄ and evaporated to provide compound 2-2 (3.3 g, 21 mmol, 70%) as a white solid.

LC/MS m/z (ES+): 158.00; HPLC [M+H]+

Step 3: To a stirred solution of compound 2-2 (3 g, 1.0 equiv, 0.02 mol) in DMF (5.0 mL) was added NaCN (1 g, 1.5 equiv, 0.03 mol) at room temperature. The resulting mixture was stirred at room temperature for 4 h. The reaction was then quenched with water and extracted with ethyl acetate. The organic layer was washed with brine, dried over Na₂SO₄ and evaporated. This resulted in compound 2-3 (2.6 g, 18 mmol, 90% yield) as a white solid.

LC/MS m/z (ES+): 149.10 [M+H]+

Step 4: To a stirred solution of compound 2-3 (2.4 g, 1.0 equiv, 16 mmol) in EtOH (25 mL) was added and AcCl (13 g, 12 mL, 10 equiv, 0.16 mol) at 0° C. After stirring at 50° C. for 10 h, HBr (3.5 g, 2.4 mL, 37% Wt, 1.0 equiv, 16 mmol) was added and the resulting mixture was stirred at room temperature for 5 h. The reaction was quenched with water and extracted with ethyl acetate. The organic layer was washed with brine, dried over Na₂SO₄ and evaporated. The resulted solution was purified using C18 flash chromatography (gradient water/MeCN) to provide compound 2-4 (1.5 g, 9.8 mmol, 60% yield) as a white solid.

LC/MS m/z (ES+)=154.05 [M+H]+

Step 5: To a stirred solution of 4-(4,4,5,5-tetramethyl-1,3,2-dioxaborolan-2-yl) aniline (200 mg, 1.0 equiv, 913 μmol), compound 2-4 (140 mg, 1.0 equiv, 913 μmol) and NaHCO₃ (0.23 g, 3.0 equiv, 2.74 mmol) in DMF (0.5 mL) was added HATU (521 mg, 1.5 Eq, 1.37 mmol) at room temperature. The resulting mixture was then stirred at room temperature for 1 h. The resulted solution was purified using C18 flash chromatography (gradient: water/MeCN) to provide compound 2-5 (170 mg, 480 μmol, 53% yield) as a white solid.

LC/MS m/z (ES+): 355.30 [M+H]+

Step 6: To a stirred solution of compound 2-5 (150 mg, 1.0 equiv, 423 μmol) and K₂CO₃ (117 mg, 2.0 equiv, 847 μmol) in DMF (1.0 mL) was added MeI (66.1 mg, 29.1 μL, 1.1 Eq, 466 μmol) at room temperature. The resulting mixture was stirred at room temperature for 1 h. The resulted solution was purified using C18 flash chromatography (gradient water/MeCN) to provide compound 2-6 (100 mg, 272 μmol, 64% yield) as a white solid.

LC/MS m/z (ES+): 369.25 [M+H]+

Step 7: To a stirred solution of compound 2-6 (100 mg, 1.0 equiv, 272 μmol), Intermediate H (137 mg, 1.2 equiv, 326 μmol), K₃PO₄ (115 mg, 2.0 equiv, 543 μmol) in DMF (1.2 mL) and H₂O (0.3 mL) were added PdCl₂(dppf)-CH₂Cl₂ (22.2 mg, 0.1 equiv, 27.2 μmol) and the resulting solution was stirred at 85° C. for 5 h under nitrogen atmosphere. The resulting solution was purified using prep-HPLC (column::Xselect CSH C18 OBD Column, gradient of water (0.1% formic acid)/MeCN). This resulted in compound 2 (29.4 mg, 50.5 μmol, 19% yield) as a white solid.

LC/MS m/z (ES+): 582.25 [M+H]+

¹H NMR (Chloroform-d, 400 MHZ) 11.18 (1H, s), 9.99 (1H, s), 8.89 (1H, s), 8.31 (1H, s), 8.13 (1H, s), 7.64 (2H, d, J=8.3 Hz), 7.56 (2H, d, J=8.3 Hz), 7.52 (1H, d, J=6.9 Hz), 7.44 (1H, d, J=7.8 Hz), 7.33 (1H, d, J=6.8 Hz), 7.23 (1H, t, J=7.8 Hz), 7.16 (1H, d, J=7.7 Hz), 6.29 (1H, t, J=6.8 Hz), 3.68 (5H, s), 3.43 (3H, s), 3.06 (3H, d, J=5.0 Hz), 1.75-1.65 (1H, m), 1.18-1.11 (2H, m), 0.96 (2H, dd, J=7.9, 3.1 Hz).

### Example 3

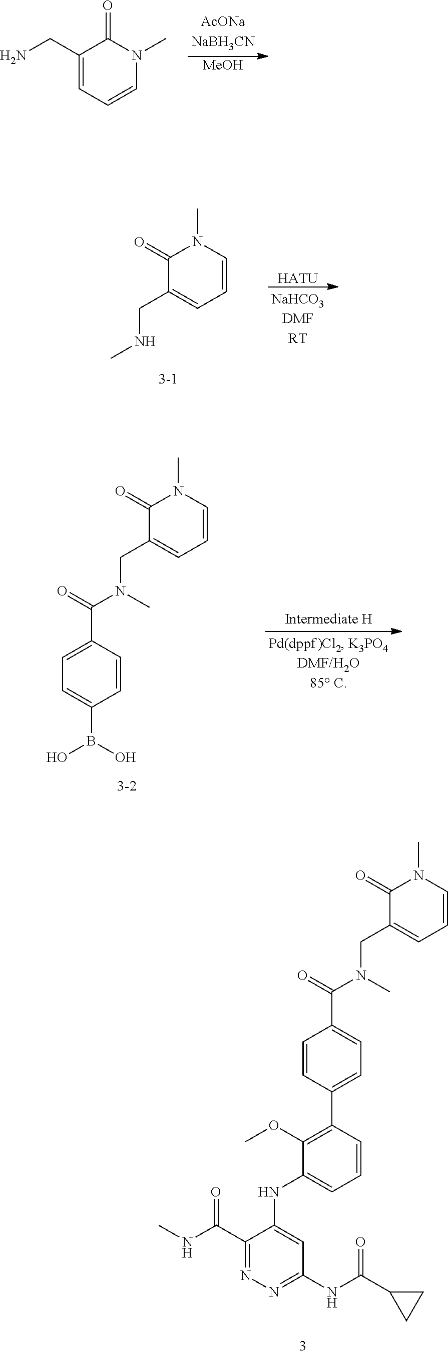

Step 1: To a stirred solution of 3-(aminomethyl)-1-methylpyridin-2 (1H)-one (200 mg, 1.0 equiv, 1.45 mmol), sodium acetate (237 mg, 2.0 equiv, 2.89 mmol) and sodium cyanoborohydride (91.0 mg, 85.3 μL, 1 Eq, 1.45 mmol) in MeOH (2 mL), The resulting mixture was stirred at 25° C. for 1 hour. LCMS was ok. The reaction was quenched with water and extracted with ethyl acetate. The organic layer was washed with brine, dried over Na₂SO₄ and evaporated to provide compound 3-1 (150 mg, 986 μmol, 68.1%) as a white solid.

LC/MS m/z (ES+)=153.10 [M+H]+

Step 2: To a stirred solution of compound 3-1 (100 mg, 1.0 equiv, 657 μmol), sodium hydrogen carbonate (166 mg, 3.0 equiv, 1.97 mmol) and HATU (375 mg, 1.5 equiv, 986 μmol) in DMF (2 mL) was added 4-(4,4,5,5-tetramethyl-1,3,2-dioxaborolan-2-yl)benzoic acid (163 mg, 1 Eq, 657 μmol) at room temperature. The resulting mixture was stirred at 25° C. for 1 hour. LCMS was ok. The reaction was quenched with water and extracted with ethyl acetate.

The organic layer was washed with brine, dried over Na₂SO₄ and evaporated. The resulted in (4-(methyl((1-methyl-2-oxo-1,2-dihydropyridin-3-yl)methyl) carbamoyl)phenyl) boronic acid (150 mg, 500 μmol, 76.1%) was white solid.

m/z (ES+) [M+H]+=383.10; HPLC tR=0.975 min.

Step 3: To a stirred solution of compound 3-2 (50 mg, 1.0 equiv, 0.17 mmol), Intermediate H (70 mg, 1.0 equiv, 0.17 mmol) and PdCl₂ (dppf) (12 mg, 0.1 Eq, 17 μmol) in DMF (1 mL):Water (0.3 mL)=3:1 at N₂. The resulting mixture was stirred at 85° C. for 1 hour. LCMS was ok. The reaction was quenched with water and extracted with EA. The organic layer was washed with brine, dried over Na₂SO₄ and evaporated. The resulting crude material was purified using prep-HPLC with following conditions: Column: XBridge Prep OBD C18 Column, 30*150 mm, 5 um; Mobile Phase A: Water (10 mmol/L NH4HCO3+0.1% NH3·H₂O), Mobile Phase B: ACN; Flow rate: 60 mL/min; Gradient: 30% B to 42% B in 7 min, 42% B; Wave Length: 220 nm; RT1 (min): 6.60; This resulted in 6-(cyclopropanecarboxamido)-4-((2-methoxy-4′-(methyl((1-methyl-2-oxo-1,2-dihydropyridin-3-yl)methyl) carbamoyl)-[1,1′-biphenyl]-3-yl)amino)-N-methylpyridazine-3-carboxamide, compound 3 (28.8 mg, 48.3 μmol, 29%) as a white amorphous solid.

m/z (ES+) [M+H]+=596.30; HPLC tR=1.411 min.

¹H NMR (400 MHz, Chloroform-d) 11.19 (s, 1H), 9.09 (s, 1H), 8.32 (s, 1H), 8.13 (s, 1H), 7.68 (d, J=7.8 Hz, 1H), 7.60 (d, J=7.8 Hz, 1H), 7.55 (d, J=7.9 Hz, 1H), 7.48 (d, J=8.0 Hz, 3H), 7.34-7.23 (m, 3H), 7.22-7.12 (m, 1H), 6.34-6.21 (m, 1H), 4.70 (s, 1H), 4.51 (s, 1H), 3.60 (d, J=15.3 Hz, 3H), 3.46-3.39 (m, 3H), 3.14 (d, J=11.1 Hz, 3H), 3.07 (d, J=5.0 Hz, 3H), 1.74 (dt, J=7.9, 3.6 Hz, 1H), 1.14 (t, J=3.8 Hz, 2H), 0.97 (dt, J=7.7, 3.5 Hz, 2H).

### Example 4

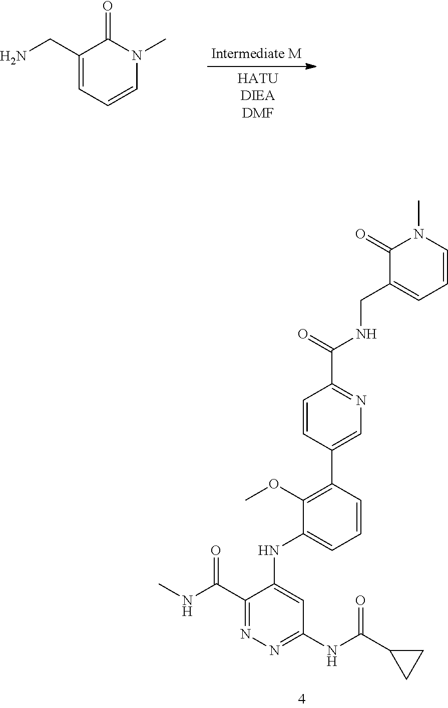

A screw cap vial was loaded with HATU (21 mg, 1.3 equiv, 56 μmol), Intermediate M (20 mg, 1.0 equiv, 43 μmol), followed by the addition of N,N-dimethylformamide (0.5 mL). To the reaction was added DIPEA (11 mg, 15 μL, 2.0 equiv, 86 μmol), and the reaction was stirred for 2 minutes after which 3-(aminomethyl)-1-methylpyridin-2 (1H)-one (12 mg, 82 μL, 1.049 molar, 2.0 equiv, 86 μmol) was added. The reaction was stirred at rt for 16 h. The reaction was then concentrated and the crude residue was purified by C18 high pressure chromatography (gradient: MeCN:H₂O+0.1% formic acid). The product containing fractions were to provide compound 4 (9.6 mg, 15 μmol, 34% yield) as a white solid.

LC/MS (ES+): m/z=583.3 [M+H]+

### Example 6

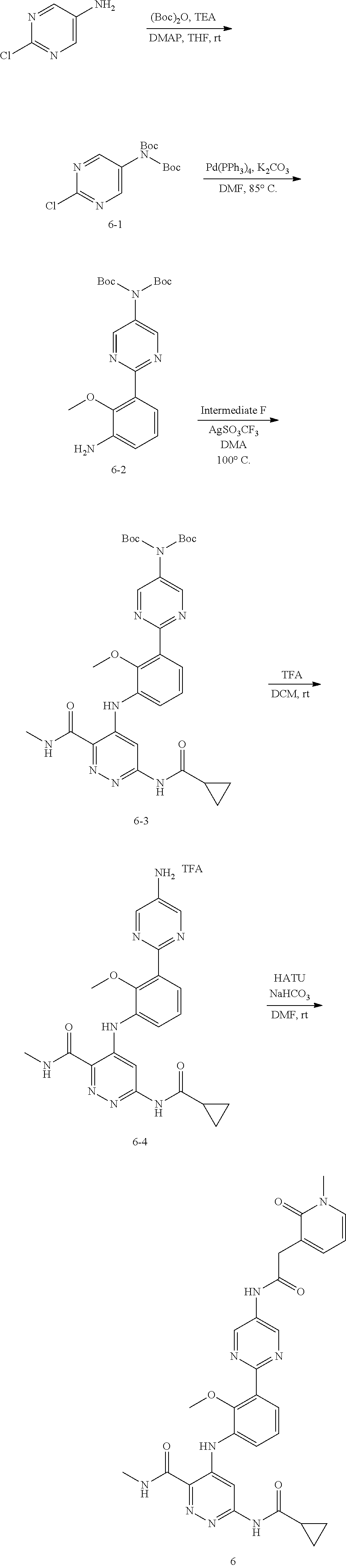 [SMILES: Nc1cnc(Cl)nc1 ; CC(C)(C)OC(=O)N(C(=O)OC(C)(C)C)c1cnc(Cl)nc1 ; COc1c(N)cccc1-c1ncc(N(C(=O)OC(C)(C)C)C(=O)OC(C)(C)C)cn1 ; CNC(=O)c1nnc(NC(=O)C2CC2)cc1Nc1cccc(-c2ncc(N(C(=O)OC(C)(C)C)C(=O)OC(C)(C)C)cn2)c1OC ; CNC(=O)c1nnc(NC(=O)C2CC2)cc1Nc1cccc(-c2ncc(N)cn2)c1OC ; CNC(=O)c1nnc(NC(=O)C2CC2)cc1Nc1cccc(-c2ncc(NC(=O)Cc3cccn(C)c3=O)cn2)c1OC]

Step 1: To a stirred solution of 2-chloropyrimidin-5-amine (450 mg, 1.0 equiv, 3.47 mmol), Boc₂O (1.52 g, 1.60 mL, 2.0 equiv, 6.95 mmol) and TEA (1.05 g, 1.45 mL, 3.0 equiv, 10.4 mmol) in THF (5 mL) was added DMAP (42.4 mg, 0.1 equiv, 347 μmol) at room temperature. The resulting mixture was stirred at room temperature for 10 h after which it was concentrated under reduced pressure and was dissolved in DMF. The resulted solution was purified using C18 flash chromatography (gradient: water/MeCN) to provide compound 6-1 (500 mg, 1.52 mmol, 43.6%) as a white solid.

LC/MS m/z (ES+): 330.00 [M+H]+

Step 2: To a stirred solution of 2-methoxy-3-(4,4,5,5-tetramethyl-1,3,2-dioxaborolan-2-yl) aniline (550 mg, 1.0 equiv, 2.21 mmol), compound 6-1 (874 mg, 1.2 equiv, 2.65 mmol) and K₂CO₃ (915 mg, 3.0 equiv, 6.62 mmol) in DMF (1.0 mL) was added Pd(Ph₃P)₄ (255 mg, 0.1 equiv, 221 μmol) at room temperature. The resulting mixture was stirred at 85° C. for 1 h under N₂. The resulted solution was purified using C18 flash chromatography (gradient: water/MeCN) to provide compound 6-2 (200 mg, 480 μmol, 22% yield) as a white solid.

LC/MS m/z (ES+): 417.30 [M+H]+

Step 3: To a stirred solution of compound 6-2 (200 mg, 1.0 equiv, 480 μmol) Intermediate F (172 mg, 1.2 equiv, 576 μmol) in DMA (2 mL) was added AgSO₃CF₃ (247 mg, 2.0 equiv, 960 μmol) at room temperature. The resulting mixture was stirred at 100° C. for 10 h. The resulting mixture was filtered, the filter cake was washed with DCM. The organic filtrate was washed with brine, dried over Na₂SO₄ and evaporated. This resulted in compound 6-3 (90 mg, 0.14 mmol, 30% yield) as a white solid.

LC/MS m/z (ES+): 535.15 [M+H-100]+

Step 4: To a stirred solution of compound 6-3 (110 mg, 1.0 equiv, 173 μmol) in DCM (1.5 mL) was added 2,2,2-trifluoroacetic acid (0.5 mL) at room temperature. The resulting mixture was stirred at room temperature for 1 h and concentrated under reduced pressure to provide compound 6-4 (100 mg, crude) as a white solid.

LC/MS m/z (ES+): 435.10 [M+H]+.

Step 5: To a stirred solution of compound 6-4 (70 mg, 1.0 equiv, 0.13 mmol), 2-(1-methyl-2-oxo-1,2-dihydropyridin-3-yl) acetic acid (22 mg, 1.0 equiv, 0.13 mmol) and NaHCO₃ (33 mg, 3.0 equiv, 0.39 mmol) in DMF (1.0 mL) was added HATU (50 mg, 1.0 equiv, 0.13 mmol) at room temperature. The resulted solution was purified using prep-HPLC (column: XBridge Prep OBD C18, gradient water/MeCN) to provide compound 6 (14.7 mg, 25.2 μmol, 19% yield) as a white solid.

LC/MS m/z (ES+): 584.25 [M+H]+

¹H NMR (DMSO-d6, 400 MHZ): 11.33 (1H, s), 10.95 (1H, s), 10.64 (1H, s), 9.13 (3H, s), 8.16 (1H, s), 7.66 (1H, dd, J=6.8, 2.1 Hz), 7.54 (2H, ddd, J=11.4, 7.9, 1.6 Hz), 7.45 (1H, dd, J=6.8, 2.0 Hz), 7.29 (1H, t, J=7.9 Hz), 6.24 (1H, t, J=6.8 Hz), 3.66 (3H, s), 3.53 (5H, d, J=53.8 Hz), 2.86 (3H, d, J=4.8 Hz), 2.09 (1H, p, J=6.2 Hz), 1.24 (OH, s), 0.83 (4H, d, J=6.1 Hz).s

### Example 7

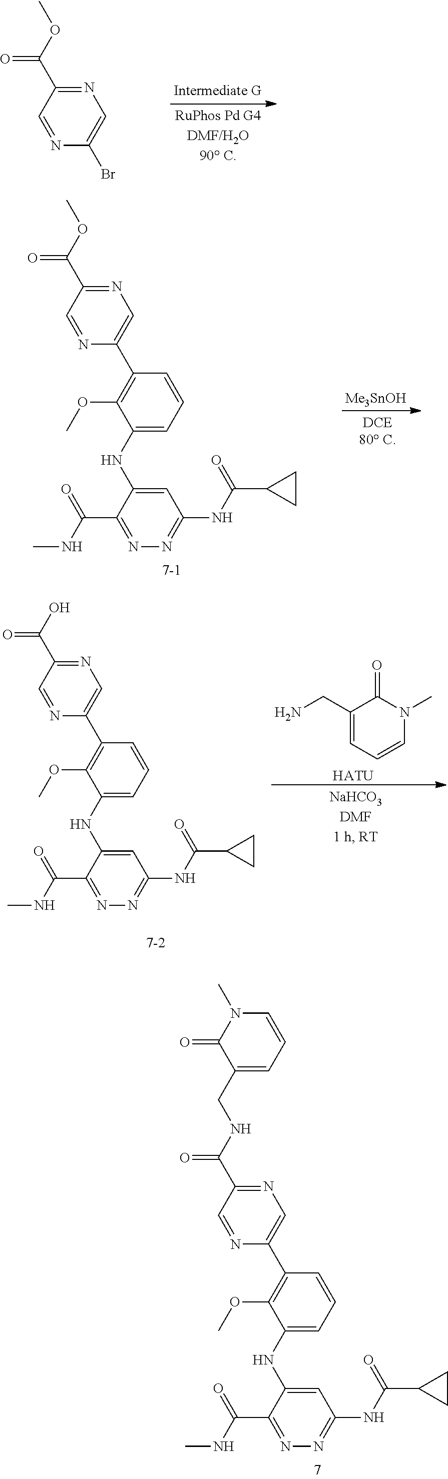

A screw cap vial was loaded with methyl 5-bromopyrazine-2-carboxylate (74 mg, 2 equiv, 0.34 mmol), potassium phosphate (73 mg, 2 equiv, 0.34 mmol), Intermediate G (80 mg, 1 Eq, 0.17 mmol) and RuPhos Pd G4 (Methanesulfonato (2-dicyclohexylphosphino-2′,6′-di-i-propoxy-1,1′-biphenyl) (2′-methylamino-1, l′-biphenyl-2-yl) palladium (II)) (22 mg, 0.15 equiv, 26 μmol). The vial was degased with nitrogen and filled with DMF (3000 μL) and water (800 mL) and the reaction was heated at 90° C. until there was determined to be no SM remaining and 90-95% desired product and 5-10% saponified product. The reaction was concentrated to provide compound 7-1 which was used directly in the next step.

LC/MS m/z (ES+): 478.2 [M+H]+

Compound 7-1 was hydrolyzed using analogous procedures in Example 11 to provide compound 7-2 and analogous amide bond formation procedures to provide compound 7.

LC/MS m/z (ES+): 584.5 [M+H]+

### Example 8

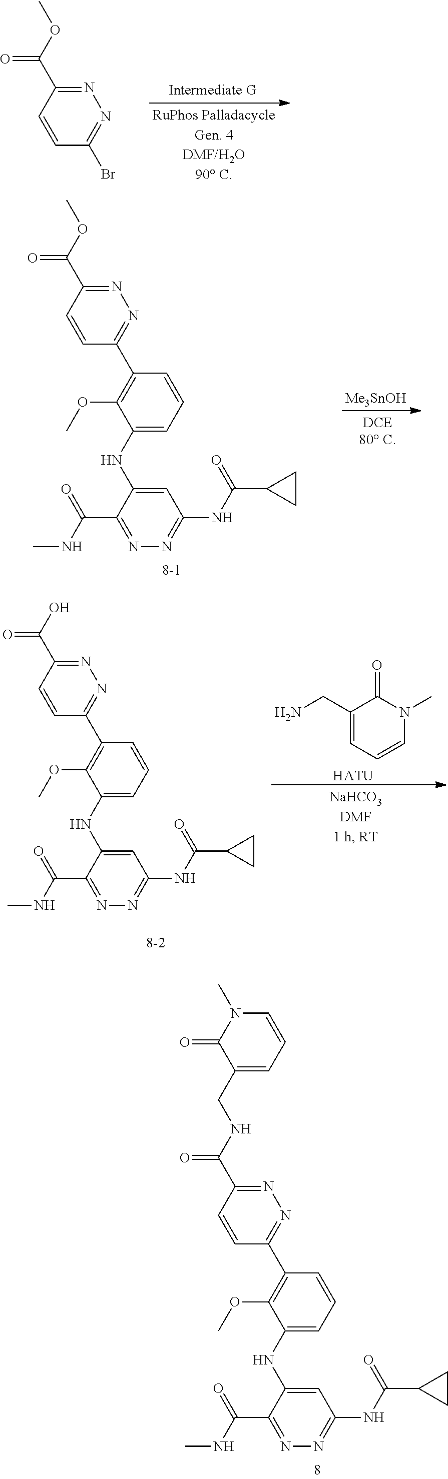

Compound 8 was prepared using analogous procedures as compound 7 in Example 7 except that methyl 6-bromopyridazine-3-carboxylate was used in place of methyl 5-bromopyrazine-2-carboxylate.

LC/MS m/z (ES+): 584.3 [M+H]+

### Example 9

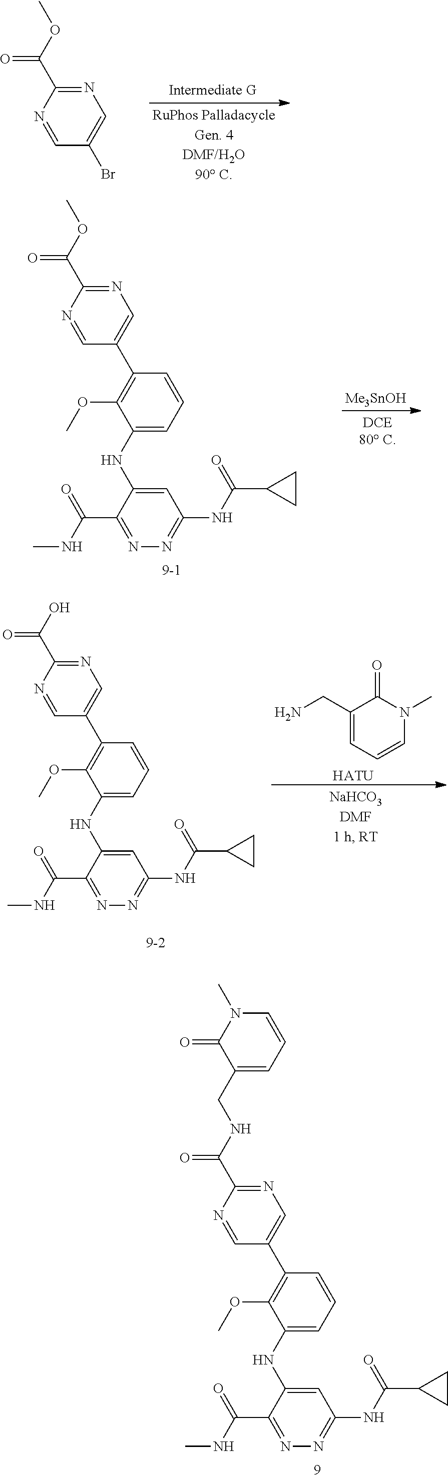

Compound 9 was prepared using analogous procedures as compound 7 in Example 7 except that methyl 6-bromopyrimidine-3-carboxylate was used in place of methyl 5-bromopyrazine-2-carboxylate.

LC/MS m/z (ES+): 584.4 [M+H]+

¹H NMR (400 MHZ, DMSO): δ 11.36 (s, 1H), 11.01 (s, 1H), 9.30 (t, J=6.2 Hz, 1H), 9.18 (s, 3H), 8.18 (s, 1H), 7.62 (ddd, J=19.8, 7.2, 2.0 Hz, 2H), 7.47-7.34 (m, 2H), 7.31 (dd, J=6.8, 1.9 Hz, 1H), 6.22 (t, J=6.8 Hz, 1H), 4.33 (d, J=6.1 Hz, 2H), 3.48 (s, 3H), 3.44 (s, 3H), 2.86 (d, J=4.8 Hz, 3H), 2.08 (s, 1H), 0.92-0.75 (m, 4H).

### Example 10

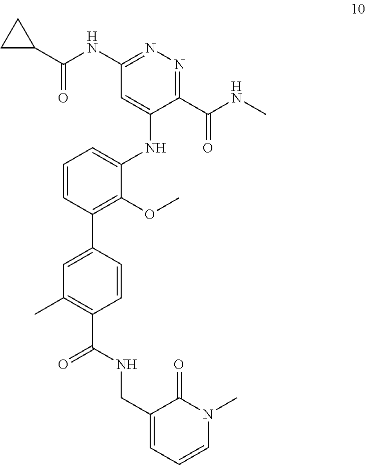 [SMILES: CNC(=O)c1nnc(NC(=O)C2CC2)cc1Nc1cccc(-c2ccc(C(=O)NCc3cccn(C)c3=O)c(C)c2)c1OC]

Compound 10 was prepared in analogous procedures to compound 1 in Example 1 except that 3-methyl-4 (4,4,5,5-tetramethyl-1,3,2-dioxaborolan-2-yl)benzoic acid was used in place of 4-(4,4,5,5-tetramethyl-1,3,2-dioxaborolan-2-yl)benzoic acid.

LC/MS m/z (ES+): need 1c/ms [M+H]+

### Example 11

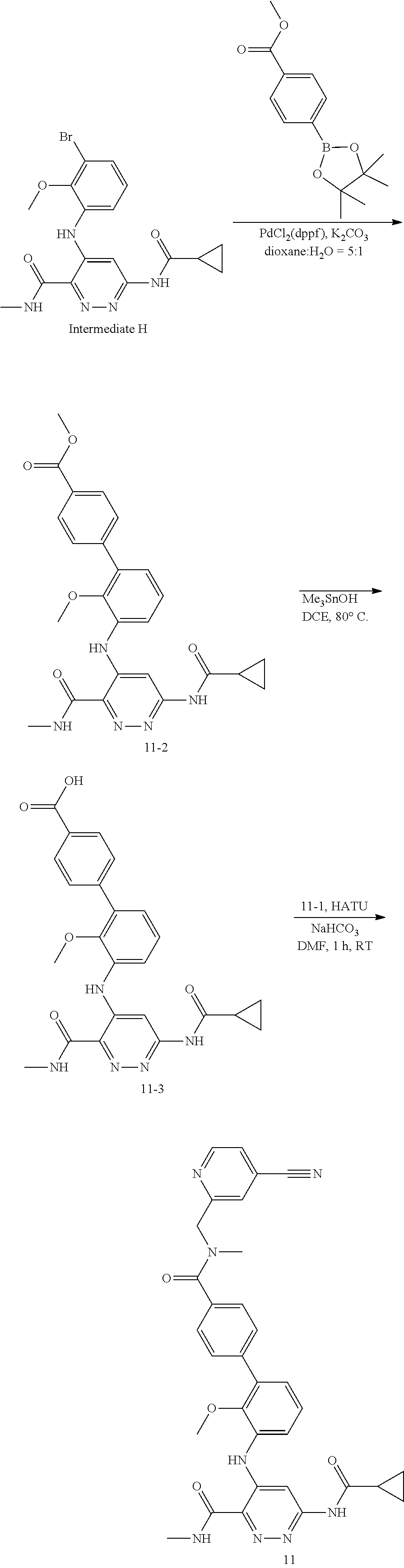 [SMILES: CNC(=O)c1nnc(NC(=O)C2CC2)cc1Nc1cccc(Br)c1OC ; COC(=O)c1ccc(B2OC(C)(C)C(C)(C)O2)cc1 ; CNC(=O)c1nnc(NC(=O)C2CC2)cc1Nc1cccc(-c2ccc(C(=O)OC)cc2)c1OC ; "Me3SnOHDCE, 80° C." ; CNC(=O)c1nnc(NC(=O)C2CC2)cc1Nc1cccc(-c2ccc(C(=O)O)cc2)c1OC ; CNC(=O)c1nnc(NC(=O)C2CC2)cc1Nc1cccc(-c2ccc(C(=O)N(C)Cc3cc(C#N)ccn3)cc2)c1OC]

### Step 1:

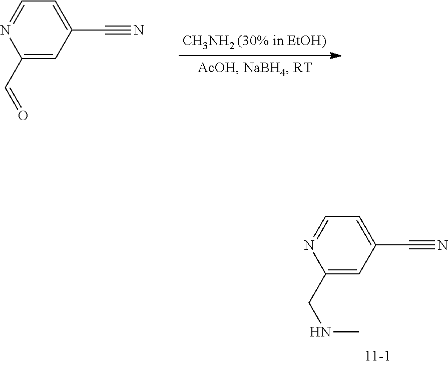

To a solution of 2-formylisonicotinonitrile (264 mg, 1.0 equiv, 2.00 mmol) in EtOH (1 mL), methylamine solution (62.1 mg, 76.2 μL, 1.0 equiv, 2.00 mmol) and acetic acid (120 mg, 115 μL, 1.0 equiv, 2.00 mmol) were added. The mixture was stirred at 25° C. for 2 hour after which NaBH₄ (151 mg, 2.0, 4.00 mmol) was added. The reaction was stirred at RT until determined to be complete by LC/MS analysis. The reaction mixture was then LCMS was ok. The reaction mixture was diluted with water (50 mL), and the aqueous phase was extracted with ethyl acetate (30 mL) three times. The combined organic layers were washed with saturated NaCl, dried over sodium sulfate, filtered, and concentrated under vacuum. The resulting solution was purified using C18 flash chromatography (gradient: water/MeCN). Concentration under vacuum provided compound 11-1 as an off-white solid.

LC/MS m/z (ES+): 148.00 [M+H]+

### Step 2:

To a solution of methyl 4-(4,4,5,5-tetramethyl-1,3,2-dioxaborolan-2-yl)benzoate (26.2 mg, 1.0 equiv, 100 μmol) in 1,4-dioxane (0.5 mL) and H₂O (0.1 mL), Intermediate H (42.0 mg, 1.0 equiv, 100 μmol) PdCl₂(dppf)-CH₂Cl₂ adduct (40.8 mg, 0.5 equiv, 50.0 μmol) and K₃PO₄ (63.6 mg, 3.0 equiv, 300 μmol) were added. The mixture was then heated to 90° C. for 2 hours until the reaction was determined to be complete by LC/MS analysis. The resulting solution was purified using C18 flash chromatography (gradient: water/MeCN). Concentration resulted in compound 11-2 as a off-white solid.

LC/MS m/z (ES+): 476.05 [M+H]+

### Step 3:

To a solution of compound 11-2 (475 mg, 1 Eq, 999 μmol) in DCE (5 mL) trimethyltinhydroxide (3.61 g, 20 equiv, 20.0 mmol) was added. The mixture was then stirred at 85° C. for 12 hour until the reaction was determined to be complete by LC/MS analysis. The resulting solution was purified using C18 flash chromatography (gradient: water/MeCN). Concentration in vacuo resulted in acid 11-3 as a off-white solid.

LC/MS m/z (ES+): 462.5 [M+H]+

### Step 4:

To a solution of 11-1 (300 mg, 1.0 equiv, 2.04 mmol) in DMF, acid 11-3 (941 mg, 1.0 equiv, 2.04 mmol), HATU (1.16 g, 1.5 equiv, 3.06 mmol), NaHCO₃ (685 mg, 4 Eq, 8.15 mmol) were added. The mixture was stirred at 25° C. for 2 hours after which the reaction was determined to be complete by LC/MS analysis. The resulting solution was purified using C18 flash chromatography (gradient: water/MeCN). Concentration in vacuo resulted in compound 11 as a off-white solid.

LC/MS m/z (ES+): 591.25 [M+H]+

¹H NMR (400 MHZ, DMSO-d6): 11.34 (s, 1H), 11.00 (s, 1H), 9.17 (d, J=5.2 Hz, 1H), 8.85 (s, 1H), 8.21 (s, 1H), 7.90-7.76 (m, 2H), 7.66 (d, J=11.2 Hz, 3H), 7.49 (s, 2H), 7.35-7.17 (m, 2H), 4.85 (s, 1H), 4.69 (s, 1H), 3.38 (s, 3H), 3.02 (d, J=37.3 Hz, 3H), 2.86 (d, J=4.8 Hz, 3H), 2.09 (d, J=7.0 Hz, 1H), 0.84 (d, J=7.2 Hz, 4H).

TABLE 2
The following compounds were prepared under analogous coupling conditions
as compound 11 in example 11.

| | | LCMS m/z | |
|---|---|---|---|
| Compound | Structure | (ES+) | ¹H NMR |
| 5 | 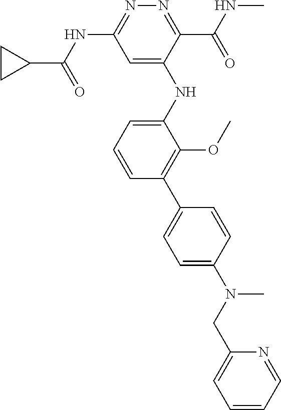 | 566.2 [M + H]+ | |
| 12 | 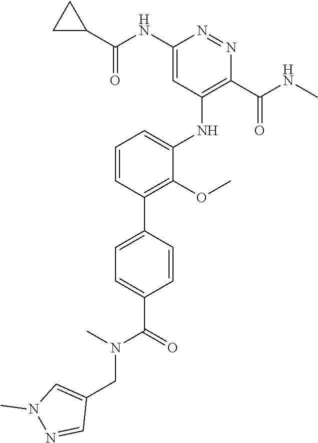 | 591.3 [M + H]+ | ¹H NMR (DMSO-d6, 400 MHz): 11.35 (1H, s), 11.00 (1H, d, J = 11.8 Hz), 9.18 (1H, d, J = 5.3 Hz), 8.85 (1H, s), 8.20 (1H, d, J = 10.8 Hz), 7.95 ^{••}C 7.76 (2H, m), 7.60 (5H, dd, J = 49.8, 20.3 Hz), 7.26 (2H, dd, J = 27.7, 13.8 Hz), 4.77 (2H, d, J = 64.7 Hz), 3.34 (4H, s), 3.02 (3H, d, J = 38.7 Hz), 2.86 (3H, d, J = 4.8 Hz), 2.10 (1H, s), 0.84 (4H, d, J = 7.3 Hz) |
| 73 | 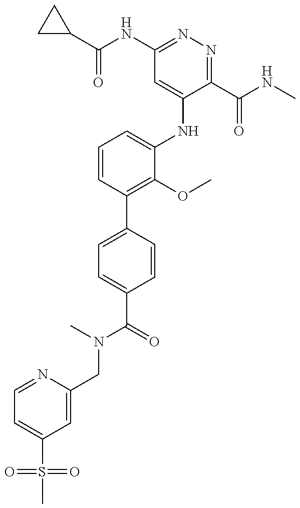 [SMILES: CNC(=O)c1nnc(NC(=O)C2CC2)cc1Nc1cccc(-c2ccc(C(=O)N(C)Cc3cc(S(C)(=O)=O)ccn3)cc2)c1OC] | 644.3 [M + H]+ | ¹H NMR (400 MHz, DMSO-d6) 11.35 (s, 1H), 10.99 (d, J = 12.2 Hz, 1H), 9.17 (d, J = 5.1 Hz, 1H), 8.94 (s, 1H), 8.20 (d, J = 10.8 Hz, 1H), 8.03-7.73 (m, 2H), 7.73-7.38 (m, 5H), 7.25 (t, J = 18.6 Hz, 2H), 4.93 (s, 1H), 4.78 (s, 1H), 3.37 (d, J = 3.7 Hz, 3H), 3.34 (d, J = 4.6 Hz, 3H), 3.04 (d, J = 37.5 Hz, 3H), 2.86 (d, J = 4.7 Hz, 3H), 2.13-2.06 (m, 1H), 0.84 (d, J = 7.3 Hz, 4H). |
| 95 | 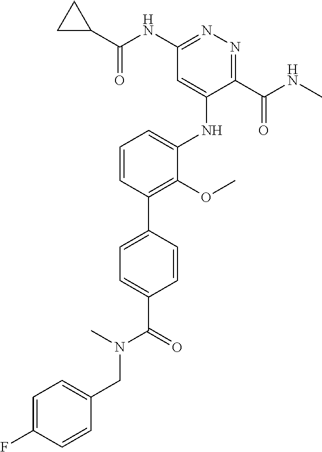 [SMILES: CNC(=O)c1nnc(NC(=O)C2CC2)cc1Nc1cccc(-c2ccc(C(=O)N(C)Cc3ccc(F)cc3)cc2)c1OC] | 583.4 [M + H]⁺ | ¹H NMR (400 MHz, Chloroform-d) 11.16 (s, 1H), 9.10 (s, 1H), 8.32 (s, 1H), 8.16 (s, 1H), 7.67 (s, 2H), 7.54 (d, J = 8.0 Hz, 2H), 7.49 (d, J = 7.8 Hz, 1H), 7.39 (s, 1H), 7.28-7.15 (m, 3H), 7.09 (t, J = 8.5 Hz, 2H), 4.77 (s, 1H), 4.58 (s, 1H), 3.44 (s, 3H), 3.07 (d, J = 5.0 Hz, 4H), 2.96 (s, 2H), 1.76 (s, 1H), 1.14 (s, 2H), 0.96 (d, J = 5.5 Hz, 2H). |
| 206* | 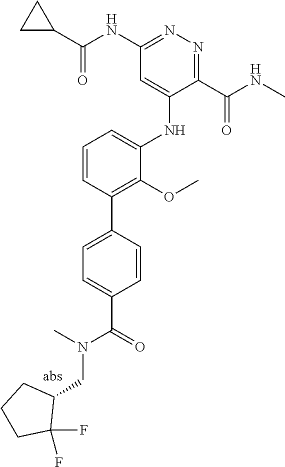 [SMILES: CNC(=O)c1nnc(NC(=O)C2CC2)cc1Nc1cccc(-c2ccc(C(=O)N(C)C[C@H]3CCCC3(F)F)cc2)c1OC] | 593.3 [M + H]+ | ¹H NMR (DMSO-d6, 400 MHz) 11.34 (1H, s), 11.01 (1H, s), 9.17 (1H, q, J = 4.8 Hz), 8.21 (1H, s), 7.68-7.61 (2H, m), 7.55-7.43 (3H, m), 7.30 (1H, t, J = 7.8 Hz), 7.23 (1H, dd, J = 7.7, 1.6 Hz), 3.64-3.35 (4H, m), 2.97 (3H, s), 2.86 (3H, d, J = 4.8 Hz), 2.68-2.56 (1H, m), 2.40-2.15 (1H, m), 2.14-2.01 (2H, m), 2.00-1.70 (3H, m), 1.67-1.42 (1H, m), 1.32-1.09 (1H, m), 0.88-0.81 (4H, m). |
| 208* | 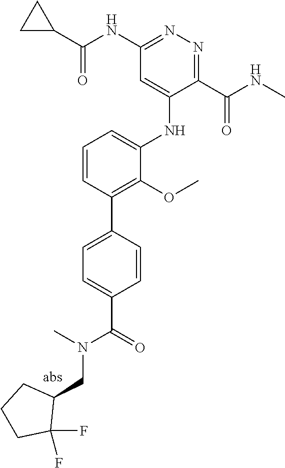 [SMILES: CNC(=O)c1nnc(NC(=O)C2CC2)cc1Nc1cccc(-c2ccc(C(=O)N(C)C[C@@H]3CCCC3(F)F)cc2)c1OC] | 593.3 [M + H]+ | ¹H NMR (DMSO-d6, 400 MHz) 11.34 (1H, s), 11.01 (1H, s), 9.17 (1H, q, J = 4.8 Hz), 8.21 (1H, s), 7.68-7.61 (2H, m), 7.55-7.43 (3H, m), 7.30 (1H, t, J = 7.8 Hz), 7.23 (1H, dd, J = 7.7, 1.6 Hz), 3.64-3.35 (4H, m), 2.97 (3H, s), 2.86 (3H, d, J = 4.8 Hz), 2.68-2.56 (1H, m), 2.40-2.15 (1H, m), 2.14-2.01 (2H, m), 2.00-1.70 (3H, m), 1.67-1.42 (1H, m), 1.32-1.09 (1H, m), 0.88-0.81 (4H, m). |
| 221 | 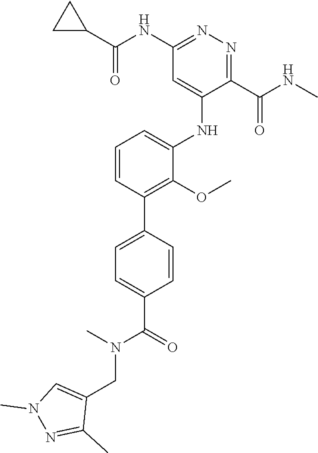 | 583.4 [M + H]+ | ¹H NMR (400 MHz, Chloroform-d) 11.24 (s, 1H), 9.30 (s, 1H), 8.33 (s, 1H), 8.11 (d, J = 5.9 Hz, 1H), 7.67 (d, J = 7.8 Hz, 2H), 7.49 (d, J = 11.5 Hz, 3H), 7.41 (s, 1H), 7.33-7.17 (m, 2H), 4.50 (d, J = 74.9 Hz, 2H), 3.86 (s, 3H), 3.44 (s, 3H), 3.23-2.78 (m, 6H), 2.33 (s, 2H), 2.11 (s, 1H), 1.76 (s, 1H), 1.15 (p, J = 4.3 Hz, 2H), 0.97 (dq, J = 7.6, 4.4, 4.0 Hz, 2H). |
| 316 | 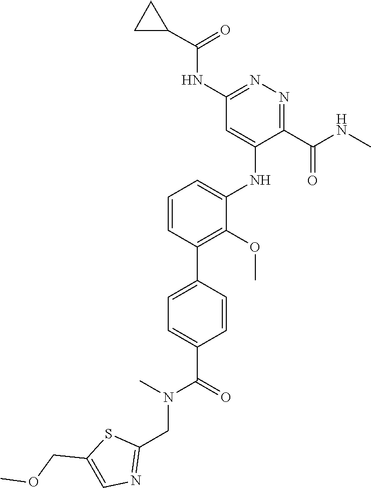 | 616.3 [M + H]+ | ¹H NMR (400 MHz, DMSO-d6) 11.34 (s, 1H), 11.00 (s, 1H), 9.17 (d, J = 4.9 Hz, 1H), 8.21 (s, 1H), 7.69 (d, J = 17.6 Hz, 3H), 7.56 (d, J = 7.8 Hz, 2H), 7.49 (d, J = 7.9 Hz, 1H), 7.30 (t, J = 7.8 Hz, 1H), 7.24 (d, J = 7.6 Hz, 1H), 4.87 (d, J = 44.7 Hz, 2H), 4.63 (s, 2H), 3.37 (s, 3H), 3.29 (s, 3H), 3.05 (s, 3H), 2.86 (d, J = 4.8 Hz, 3H), 2.11 (q, J = 6.1 Hz, 1H), 0.88-0.81 (m, 4H). |

*Absolute stereochemistry arbitrarily assigned.

### Example 13

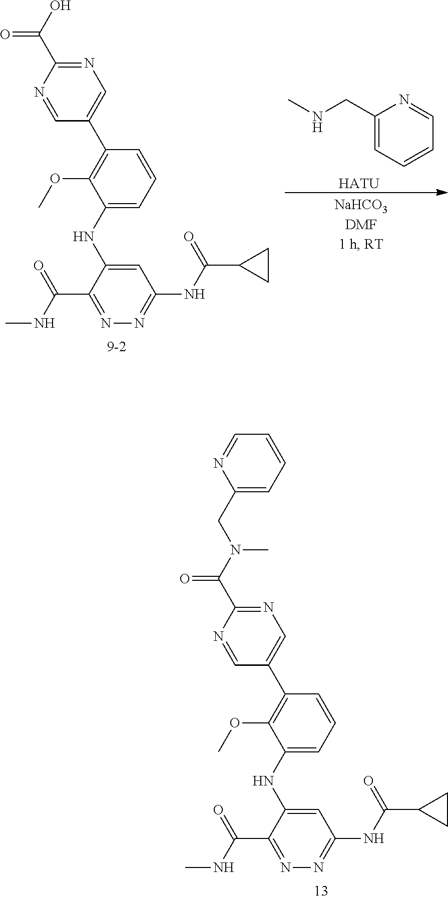

Compound 13 was prepared using standard coupling procedures in example 18.

LC/MS m/z (ES+): 568.2 [M+H]+

### Example 14

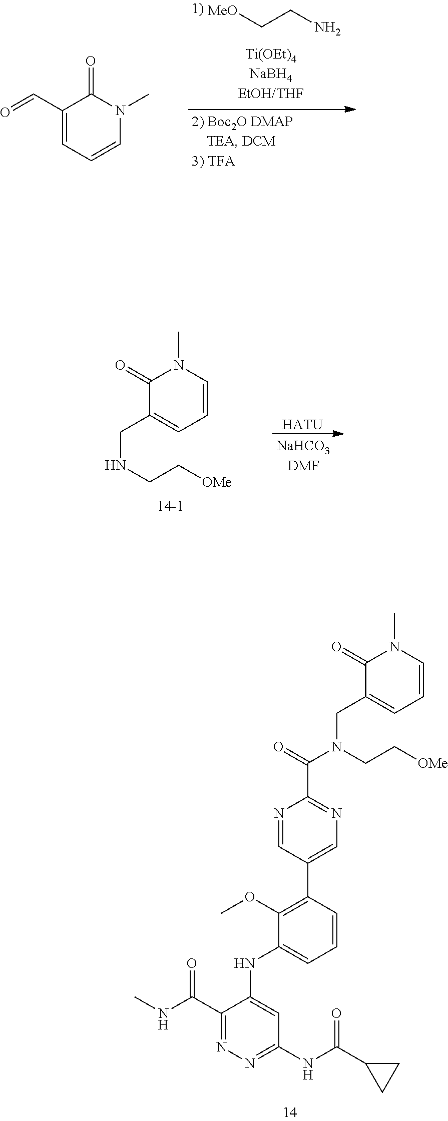

Step 1:1-Amino-2-methoxyethane (548 mg, 631 μL, 2.0 equiv, 7.29 mmol) in 3 mL of THF and and titaniumethoxide (1.14 mL, 1.5 equiv, 5.47 mmol) and the reaction heated to 60° C. for 16 hrs. The reaction was concentrated, redissolved in 6 mL of THF and 1 mL of EtOH after which sodium borohydride (414 mg, 3 equiv, 10.9 mmol) was added. The reaction stirred at rt, then quenched with 2 mL of AcOH and EtOAc (1:1 v/v). The reaction was then diluted with EtOAc and neutralized with saturated NaHCO₃. The reaction was filtered, and the filtrate was partitioned between water and organics. The organics were washed with brine and the combined aqueous layer was re-extracted with EtOAc. The combined organics dried and concentrated. The crude oil was redissolved in DCM (2 mL) after which triethylamine (3 equiv), Boc₂O (1.2 equiv) and cat DMAP (10 mol %) were added. The reaction was stirred for 1 hr at RT after which the reaction quenched with MeOH, dry loaded on silica and purified by flash silica gel chromatography (gradient MeOH/DCM). The product fractions were combined and concentrated. The amine was stirred in TFA (2 mL) for 30 min at RT and then concentrated to provide the desired amine 14-1 which was used directly in the next step.

Step 2: To compound 8-2 (acid) (30 mg, 1 equiv, 65 μmol), HATU (37 mg, 1.5 equiv, 97 μmol), sodium bicarbonate (27 mg, 5.0 equiv, 0.32 mmol) and compound 14-1 (13 mg, 1 Eq, 65 μmol) was added 0.3 mL of DMF and reaction stirred overnight. The reaction diluted in 3 mL of DMSO, filtered and purified by reverse phase HPLC (gradient MeCN/water). The product fractions were combined and concentrated to a white solid to provide compound 14.

LC/MS m/z (ES+): 642.0 [M+H]+

### Example 15

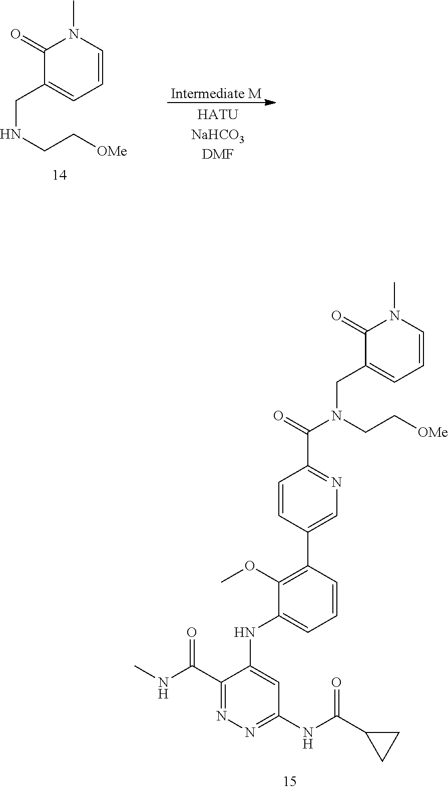 [SMILES: COCCNCc1cccn(C)c1=O ; "Intermediate M" ; CNC(=O)c1nnc(NC(=O)C2CC2)cc1Nc1cccc(-c2ccc(C(=O)N(CCOC)Cc3cccn(C)c3=O)nc2)c1OC]

Compound 15 was prepared in analogous fashion to compound 14 in Example 14 except that Intermediate M was used in place of compound 8-2.

LC/MS m/z (ES+): 641.6 [M+H]+

### Example 16

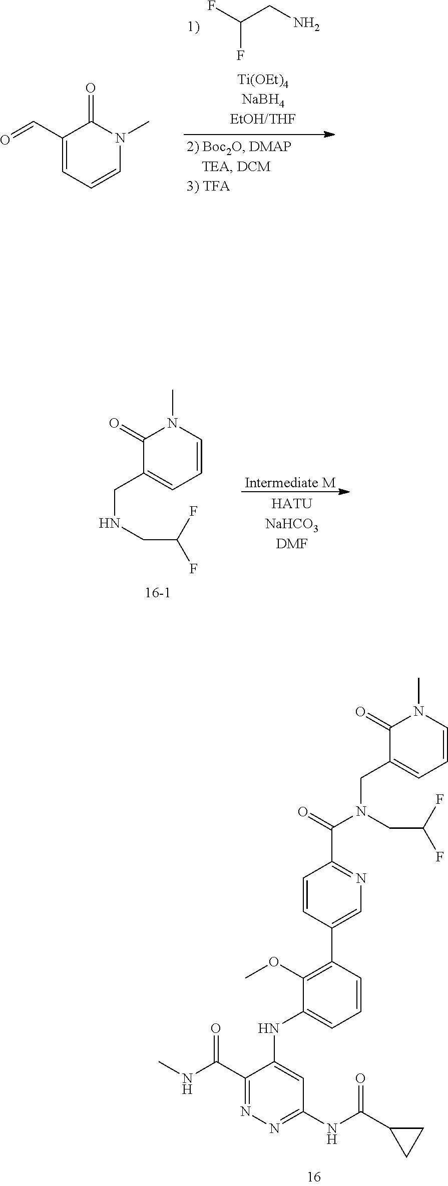

Compound 16 was prepared in analogous fashion to compound 14 in Example 14 except that 2,2-difluoroethan-1-amine was used in place of 1-amino-2-methoxyethane and Intermediate M was used in place of compound 8-2.

LC/MS m/z (ES+): 647.6 [M+H]+

### Example 18

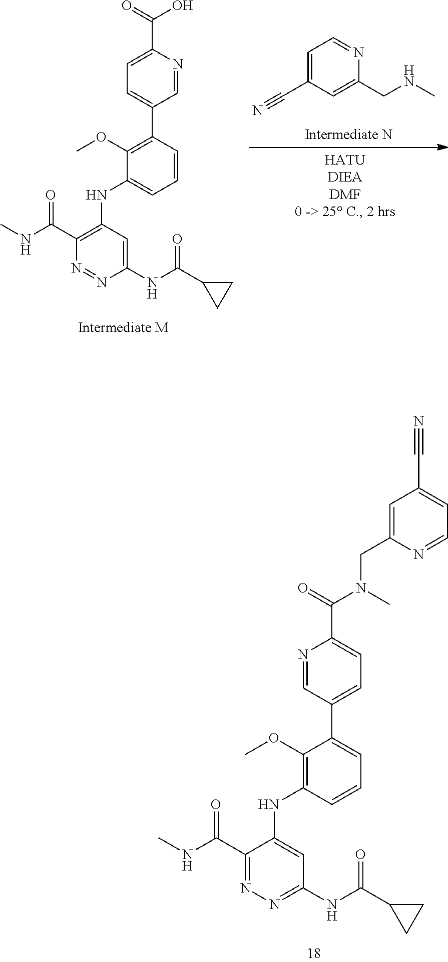 [SMILES: CNC(=O)c1nnc(NC(=O)C2CC2)cc1Nc1cccc(-c2ccc(C(=O)O)nc2)c1OC ; CNCc1cc(C#N)ccn1 ; CNC(=O)c1nnc(NC(=O)C2CC2)cc1Nc1cccc(-c2ccc(C(=O)N(C)Cc3cc(C#N)ccn3)nc2)c1OC]

To a solution of Intermediate M (33.0 g, 66.1 mmol, 1.0 equiv, 2 HCl) and Intermediate N (9.73 g, 66.1 mmol, 1.0 equiv) in DMF (300 mL) was added HATU (37.7 g, 99.2 mmol, 1.5 equiv) and DIEA (42.7 g, 330 mmol, 57.0 mL, 5.0 eqiv) at 0° C., after which the mixture was allowed to warmed to 25° C. and stirred at 25° C. for 2 hrs. After the reaction was determined to be complete by LC/MS analysis, the reaction mixture was poured into H₂O (1.00 L), the aqueous phase was extracted with ethyl acetate (300 mL×3). The combined organic phase was washed with brine (500 mL×2), dried with anhydrous Na₂SO₄, filtered and concentrated under vacuum to give a crude product. The crude was triturated with ethyl acetate (200 mL) at 25° C. for 2 hrs, filtered and the filter cake was dried over vacuum at 50° C. The residue was triturated with MeCN (200 mL) at 25° C. for 16 hrs and filtered. The resulting filter cake was dried over vacuum at 50° C. to provide compound 18 (22.0 g, 36.5 mmol, 55% yield) was obtained as an off-white solid.

LCMS m/z (ES+): 592.1 (M+H)+.

1H NMR (400 MHZ, CDCl₃): δ 11.12 (s, 1H), 9.46 (s, 1H), 8.84-8.76 (m, 2H), 8.31 (d, J=7.2 Hz, 1H), 8.13-8.10 (m, 2H), 7.84-7.72 (m, 2H), 7.54-7.45 (m, 2H), 7.33-7.28 (m, 1H), 7.22-7.15 (m, 1H), 5.03 (d, J=24.4 Hz, 2H), 3.47 (d, J=15.6 Hz, 3H), 3.31 (d, J=60.8 Hz, 3H), 3.06-3.04 (m, 3H), 1.84-1.78 (m, 1H), 1.15-1.09 (m, 2H), 0.95-0.90 (m, 2H).

TABLE 3
The following compounds were prepared under analogous conditions as
compound 18 in example 18:

| | | LCMS m/z | |
|---|---|---|---|
| Compound | Structure | (ES+) | ¹H NMR |
| 17 | 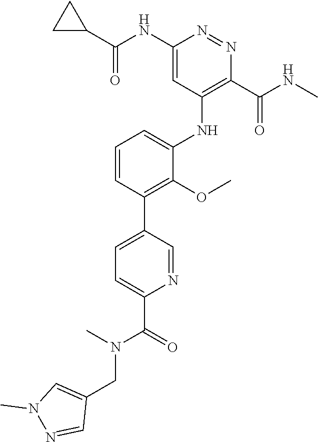 | 570.4 [M + H]+ | |
| 19 | 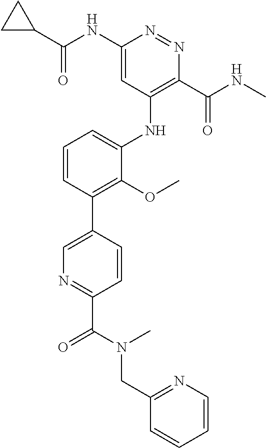 [SMILES: CNC(=O)c1nnc(NC(=O)C2CC2)cc1Nc1cccc(-c2ccc(C(=O)N(C)Cc3ccccn3)nc2)c1OC] | 567.5 [M + H]+ | |
| 23 | 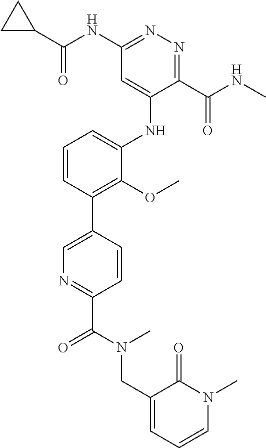 [SMILES: CNC(=O)c1nnc(NC(=O)C2CC2)cc1Nc1cccc(-c2ccc(C(=O)N(C)Cc3cccn(C)c3=O)nc2)c1OC] | 597.4 [M + H]+ | ¹H NMR (500 MHz, DMSO) δ 11.34 (d, J = 4.8 Hz, 1H), 10.99 (d, J = 11.7 Hz, 1H), 9.15 (p, J = 4.9 Hz, 1H), 8.77 (dd, J =17.4, 2.2 Hz, 0H), 8.17-8.06 (m, 2H), 7.75- 7.60 (m, 2H), 7.52 (ddd, J = 12.0, 6.7, 2.9 Hz, 1H), 7.31 (dtd, J = 15.4, 7.8, 4.0 Hz, 3H), 6.24 (dt, J = 27.5, 6.8 Hz, 1H), 4.46 (d, J = 12.4 Hz, 2H), 3.47 (s, 1H), 3.41-3.34 (m, 4H), 3.03 (s, 1H), 2.95 (s, 1H), 2.85 (dd, J = 4.9, 2.2 Hz, 3H), 2.07 (tq, J = 9.0, 5.0 Hz, 1H), 0.82 (hept, J = 3.4 Hz, 4H). |
| 26 | 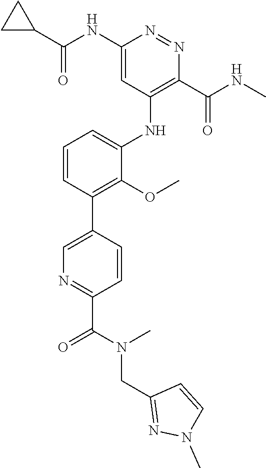 [SMILES: CNC(=O)c1nnc(NC(=O)C2CC2)cc1Nc1cccc(-c2ccc(C(=O)N(C)Cc3ccn(C)n3)nc2)c1OC] | 570.4 [M + H]+ | ¹H NMR (400 MHz, DMSO) δ 11.33 (s, 1H), 10.98 (d, J = 3.7 Hz, 1H), 9.16 (d, J = 5.1 Hz, 1H), 8.76 (ddd, J = 4.3, 2.2, 0.9 Hz, 1H), 8.17 (d, J = 2.9 Hz, 1H), 8.11 (ddd, J = 8.1, 3.9, 2.0 Hz, 1H), 7.68 (dt, J = 8.1, 1.2 Hz, 1H), 7.61 (dd, J = 21.7, 2.2 Hz, 1H), 7.57-7.45 (m, 1H), 7.39- 7.24 (m, 2H), 6.14 (dd, J = 32.1, 2.2 Hz, 1H), 4.57 (d, J = 29.9 Hz, 2H), 3.78 (d, J = 20.7 Hz, 3H), 3.38 (d, J = 4.2 Hz, 3H), 2.95 (d, J = 2.2 Hz, 3H), 2.85 (d, J = 4.8 Hz, 3H), 2.15-2.03 (m, J = 2.7 Hz, 1H), 0.94-0.73 (m, 4H). |
| 30 | 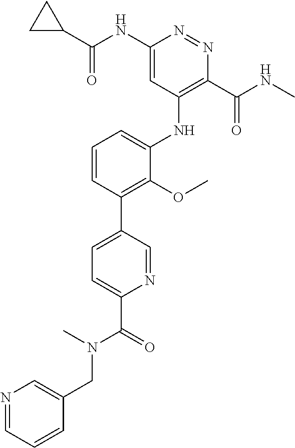 [SMILES: CNC(=O)c1nnc(NC(=O)C2CC2)cc1Nc1cccc(-c2ccc(C(=O)N(C)Cc3cccnc3)nc2)c1OC] | 567.5 [M + H]+ | ¹H NMR (400 MHz, DMSO) δ 11.35 (s, 1H), 10.99 (d, J = 6.9 Hz, 1H), 9.19 (s, 1H), 8.79 (d, J = 1.8 Hz, 1H), 8.70 (d, J = 33.5 Hz, 1H), 8.57 (s, 1H), 8.26-8.08 (m, 2H), 7.90 (s, 1H), 7.85-7.68 (m, 1H), 7.63-7.48 (m, 2H), 7.33 (t, J = 7.4 Hz, 2H), 4.75 (d, J = 23.2 Hz, 2H), 3.39 (d, J = 11.9 Hz, 3H), 2.99 (d, J = 18.1 Hz, 3H), 2.91-2.80 (m, 3H), 2.15-2.03 (m, J = 2.7 Hz, 1H), 0.83 (d, J = 4.5 Hz, 1H). |
| 31 | 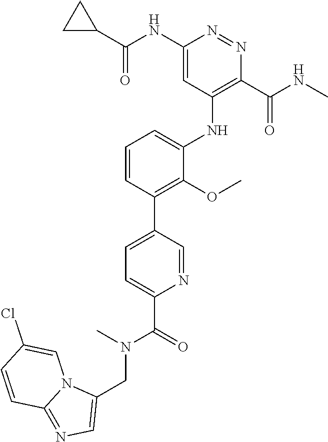 [SMILES: CNC(=O)c1nnc(NC(=O)C2CC2)cc1Nc1cccc(-c2ccc(C(=O)N(C)Cc3cnc4ccc(Cl)cn34)nc2)c1OC] | 640.6 [M + H]+ | ¹H NMR (400 MHz, DMSO) δ 11.34 (s, 1H), 10.99 (s, 1H), 9.17 (d, J = 4.9 Hz, 1H), 8.84 (s, 1H), 8.75 (s, 1H), 8.18 (s, 1H), 8.13 (s, 1H), 7.78 (s, 1H), 7.75-7.62 (m, 2H), 7.59-7.45 (m, 1H), 7.41- 7.25 (m, 3H), 5.11 (s, 2H), 3.39 (s, 3H), 2.95 (s, 2H), 2.85 (d, J = 4.8 Hz, 3H), 2.08 (q, J = 6.2 Hz, 1H), 0.82 (d, J = 4.5 Hz, 4H). |
| 40 | 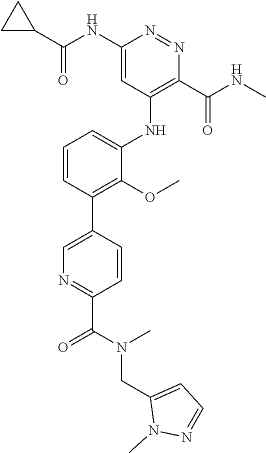 [SMILES: CNC(=O)c1nnc(NC(=O)C2CC2)cc1Nc1cccc(-c2ccc(C(=O)N(C)Cc3ccnn3C)nc2)c1OC] | 570.3 [M + H]+ | |
| 45 | 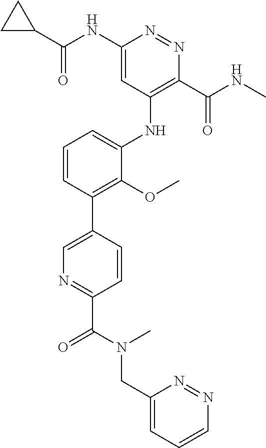 [SMILES: CNC(=O)c1nnc(NC(=O)C2CC2)cc1Nc1cccc(-c2ccc(C(=O)N(C)Cc3cccnn3)nc2)c1OC] | 568.4 [M + H ]+ | |
| 46 | 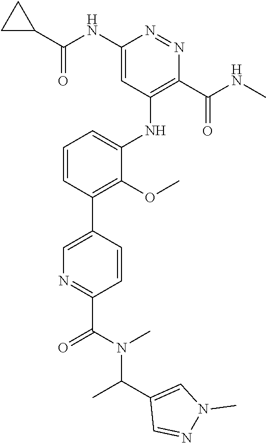 [SMILES: CNC(=O)c1nnc(NC(=O)C2CC2)cc1Nc1cccc(-c2ccc(C(=O)N(C)C(C)c3cnn(C)c3)nc2)c1OC] | 584.4 (M + H)+ | |
| 50 | 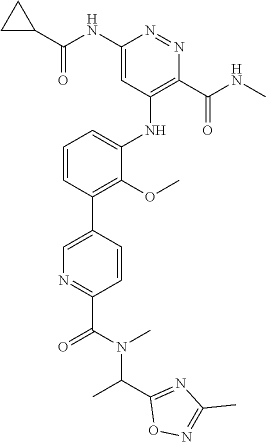 [SMILES: CNC(=O)c1nnc(NC(=O)C2CC2)cc1Nc1cccc(-c2ccc(C(=O)N(C)C(C)c3nc(C)no3)nc2)c1OC] | 586.5 [M + H]+ | |
| 51 | 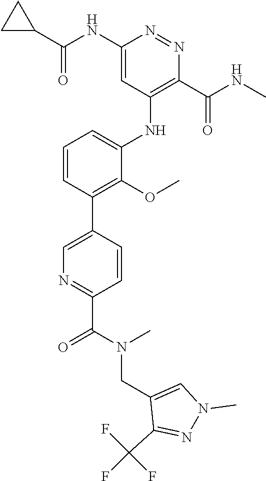 [SMILES: CNC(=O)c1nnc(NC(=O)C2CC2)cc1Nc1cccc(-c2ccc(C(=O)N(C)Cc3cn(C)nc3C(F)(F)F)nc2)c1OC] | 638.2 [M + H]+ | |
| 52 | 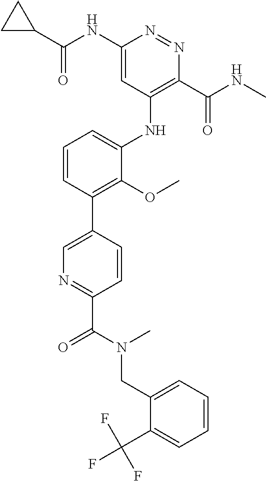 [SMILES: CNC(=O)c1nnc(NC(=O)C2CC2)cc1Nc1cccc(-c2ccc(C(=O)N(C)Cc3ccccc3C(F)(F)F)nc2)c1OC] | 634.2 [M + H]+ | |
| 54 | 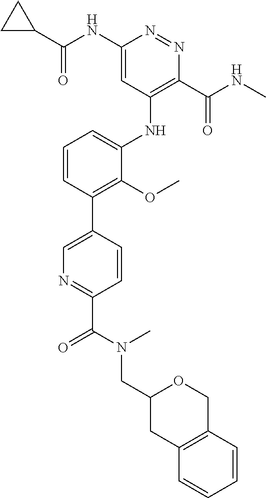 [SMILES: CNC(=O)c1nnc(NC(=O)C2CC2)cc1Nc1cccc(-c2ccc(C(=O)N(C)CC3Cc4ccccc4CO3)nc2)c1OC] | 622.5 [M + H]+ | |
| 57 | 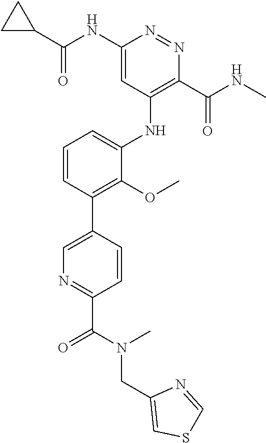 [SMILES: CNC(=O)c1nnc(NC(=O)C2CC2)cc1Nc1cccc(-c2ccc(C(=O)N(C)Cc3cscn3)nc2)c1OC] | 573.2 [M + H]+ | ¹H NMR (500 MHz, DMSO) δ 11.33 (d, J = 2.0 Hz, 1H), 10.99 (d, J = 5.3 Hz, 1H), 9.16 (d, J = 5.1 Hz, 1H), 9.09 (dd, J = 34.1, 1.9 Hz, 1H), 8.83-8.65 (m, 1H), 8.18 (d, J = 5.6 Hz, 1H), 8.12 (ddd, J = 19.4, 8.1, 2.2 Hz, 1H), 7.71 (dd, J = 9.7, 8.1 Hz, 1H), 7.64-7.49 (m, 2H), 7.39- 7.29 (m, 2H), 4.83 (d, J = 15.4 Hz, 2H), 3.39 (d, J = 11.3 Hz, 3H), 3.04 (d, J = 24.4 Hz, 3H), 2.95-2.78 (m, 3H), 2.15-2.00 (m, 1H), 0.83 (d, J = 7.0 Hz, 4H) |
| 58 | 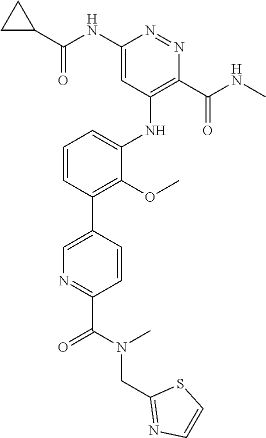 [SMILES: CNC(=O)c1nnc(NC(=O)C2CC2)cc1Nc1cccc(-c2ccc(C(=O)N(C)Cc3nccs3)nc2)c1OC] | 573.2 [M + H]+ | |
| 59 | 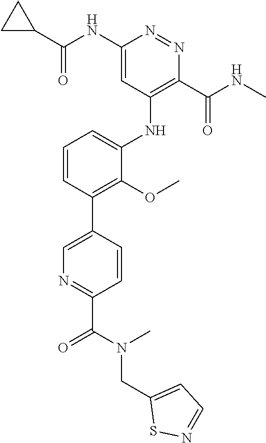 [SMILES: CNC(=O)c1nnc(NC(=O)C2CC2)cc1Nc1cccc(-c2ccc(C(=O)N(C)Cc3ccns3)nc2)c1OC] | 573.1 [M + H]+ | |
| 60 | 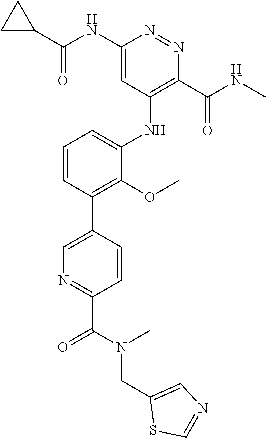 [SMILES: CNC(=O)c1nnc(NC(=O)C2CC2)cc1Nc1cccc(-c2ccc(C(=O)N(C)Cc3cncs3)nc2)c1OC] | 573.2 [M + H]+ | |
| 65* | 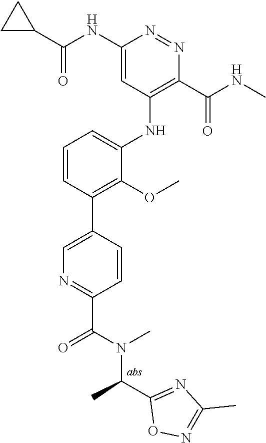 [SMILES: CNC(=O)c1nnc(NC(=O)C2CC2)cc1Nc1cccc(-c2ccc(C(=O)N(C)[C@H](C)c3nc(C)no3)nc2)c1OC] | 586.5 [M + H]+ | |
| 71 | 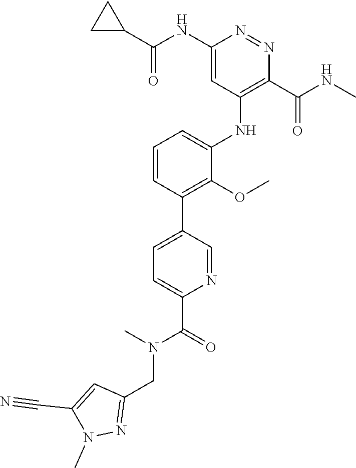 [SMILES: CNC(=O)c1nnc(NC(=O)C2CC2)cc1Nc1cccc(-c2ccc(C(=O)N(C)Cc3cc(C#N)n(C)n3)nc2)c1OC] | 595.4 [M + H]+ | ¹H NMR (400 MHz, DMSO) δ 11.35 (s, 1H), 10.99 (d, J = 2.5 Hz, 1H), 9.17 (d, J = 5.1 Hz, 1H), 8.78 (ddd, J = 5.9, 2.2, 0.9 Hz, 1H), 8.18 (d, J = 3.6 Hz, 1H), 8.13 (dt, J = 8.1, 2.3 Hz, 1H), 7.72 (dd, J = 8.1, 0.8 Hz, 1H), 7.53 (dt, J = 6.1, 2.5 Hz, 1H), 7.43-7.25 (m, 2H), 7.10 (d, J = 1.9 Hz, 1H), 4.66 (d, J = 18.4 Hz, 2H), 3.99 (d, J = 19.7 Hz, 3H), 3.39 (d, J = 3.2 Hz, 3H), 3.00 (d, J = 9.8 Hz, 3H), 2.86 (d, J = 4.8 Hz, 3H), 2.09 (qt, J = 6.9, 3.8 Hz, 1H), 0.83 (q, J = 3.0, 2.6 Hz, 4H). |
| 72 | 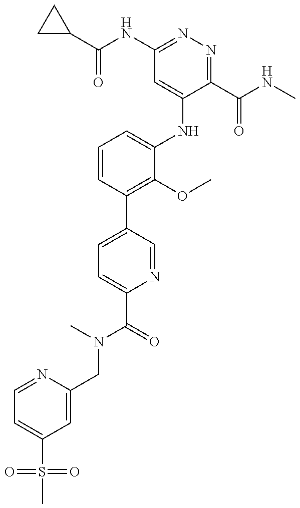 [SMILES: CNC(=O)c1nnc(NC(=O)C2CC2)cc1Nc1cccc(-c2ccc(C(=O)N(C)Cc3cc(S(C)(=O)=O)ccn3)nc2)c1OC] | 645.3 [M + H]+ | ¹H NMR (DMSO-d6, 400 MHz): 11.35 (1H, d, J = 5.0 Hz), 11.00 (1H, d, J = 11.4 Hz), 9.18 (1H, p, J = 4.6 Hz), 8.98-8.63 (2H, m), 8.26-8.05 (2H, m), 7.93-7.79 (2H, m), 7.78-7.70 (1H, m), 7.53 (1H, ddd, J = 12.0, 7.2, 2.4 Hz), 7.40-7.25 (2H, m), 4.97 (2H, d, J = 11.9 Hz), 3.40 (3H, d, J = 17.0 Hz), 3.37-3.31 (3H, m), 3.11 (3H, d, J = 37.7 Hz), 2.86 (3H, dd, J = 4.8, 3.0 Hz), 2.16-2.04 (1H, m), 0.84 (4H, ddd, J = 8.3, 4.5, 2.5 Hz). |
| 80 | 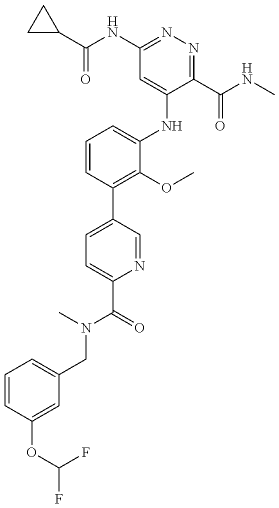 [SMILES: CNC(=O)c1nnc(NC(=O)C2CC2)cc1Nc1cccc(-c2ccc(C(=O)N(C)Cc3cccc(OC(F)F)c3)nc2)c1OC] | 532.3 [M + H]+ | ¹H NMR (DMSO-d6, 400 MHz) 0.83 (4H, s), 2.10 (1H, s), 2.86 (3H, dd), 2.97 (3H, d), 3.39 (3H, d), 4.73 (2H, d), 7.02-7.60 (8H, m), 7.75 (1H, dd), 8.03-8.28 (2H, m), 8.78 (1H, d), 9.17 (1H, s), 10.99 (1H, d), 11.35 (1H, s) |
| 81 | 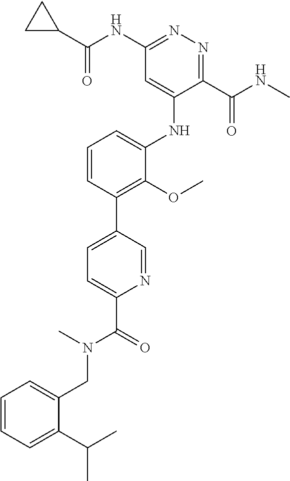 [SMILES: CNC(=O)c1nnc(NC(=O)C2CC2)cc1Nc1cccc(-c2ccc(C(=O)N(C)Cc3ccccc3C(C)C)nc2)c1OC] | 608.3 [M + H]+ | ¹H NMR (DMSO-d6, 400 MHz) 0.84 (4H, d), 1.05 (3H, d), 1.23 (4H, d), 2.10 (1H, s), 2.81-2.98 (7H, m), 3.41 (2H, s), 4.81 (2H, d), 7.10-7.46 (6H, m), 7.49-7.58 (1H, m), 7.69 (1H, dd), 8.12-8.22 (2H, m), 8.76 (1H, d), 9.18 (0H, d), 10.98 (1H, d), 11.34 (1H, d) |
| 82 | 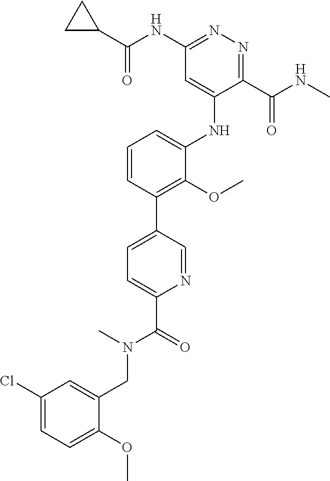 [SMILES: CNC(=O)c1nnc(NC(=O)C2CC2)cc1Nc1cccc(-c2ccc(C(=O)N(C)Cc3cc(Cl)ccc3OC)nc2)c1OC] | 630.3 [M + H]+ | ¹H NMR (DMSO-d6, 400 MHz) 0.84 (4H, t), 2.10 (1H, s), 2.87 (3H, dd), 2.98 (3H, d0, 3.40 (3H, d), 3.76 (3H, d), 4.67 (2H, d), 6.99 (1H, d), 7.10 (1H, d), 7.22-7.35 (3H, m), 7.53 (1H, s), 7.73 (1H, dd), 8.07-8.25 (2H, m), 8.77 (1H, d), 9.17 (1H, s), 10.99 (1H, d), 11.34 (1H, s) |
| 83 | 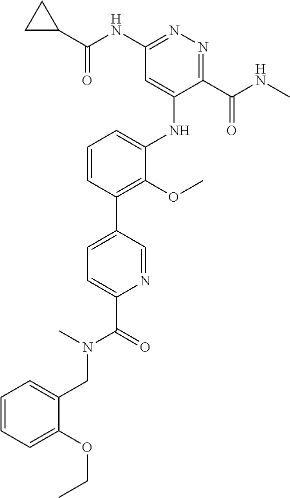 [SMILES: CCOc1ccccc1CN(C)C(=O)c1ccc(-c2cccc(Nc3cc(NC(=O)C4CC4)nnc3C(=O)NC)c2OC)cn1] | 610.5 [M + H]+ | ¹H NMR (DMSO-d6, 400 MHz) 0.84 (4H, d), 1.30 (3H, dt), 2.09 (1H, s), 2.86 (3H, dd), 2.96 (3H, d), 3.39 (3H, d), 3.97 (1H, q), 4.10 (1H, q), 4.69 (2H, s), 6.90-7.06 (2H, m), 7.20-7.36 (4H, m), 7.49- 7.57 (1H, m), 7.71 (1H, dd), 8.10-8.21 (2H, m), 8.71-8.83 (1H, m), 9.19 (1H, s), 11.00 (1H, d), 11.36 (1H, d) |
| 84 | 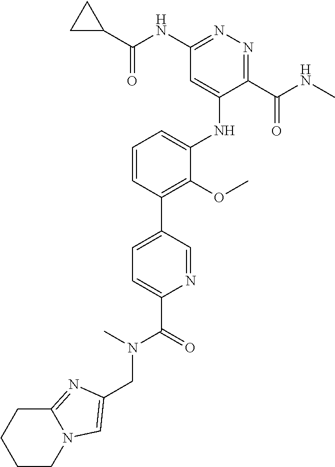 [SMILES: CNC(=O)c1nnc(NC(=O)C2CC2)cc1Nc1cccc(-c2ccc(C(=O)N(C)Cc3cn4c(n3)CCCC4)nc2)c1OC] | 610.3 [M + H]+ | ¹H NMR (DMSO-d6, 400 MHz) 0.01 (4H, s), 0.84 (4H, d), 2.06-2.14 (1H, m), 2.62- 2.74 (2H, m), 2.86 (3H, d), 2.99 (3H, d), 3.40 (3H, d), 3.89 (2H, dt), 4.45 (2H, d), 6.87 (1H, d), 7.29-7.39 (2H, m), 7.54 (1H, d), 7.70 (1H, dd), 8.12 (1H, d), 8.20 (1H, d), 8.74-8.81 (1H, m), 9.19 (1H, d), 11.00 (1H, s), 11.36 (1H, s) |
| 85 | 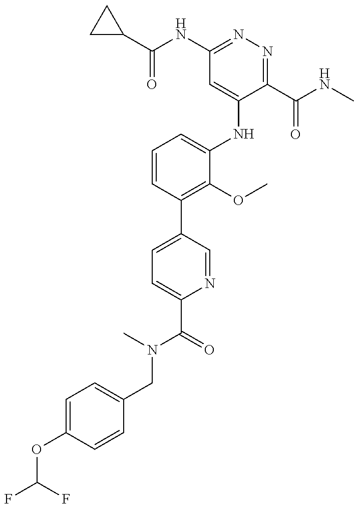 [SMILES: CNC(=O)c1nnc(NC(=O)C2CC2)cc1Nc1cccc(-c2ccc(C(=O)N(C)Cc3ccc(OC(F)F)cc3)nc2)c1OC] | 632.3 [M + H]+ | ¹H NMR (DMSO-d6, 400 MHz): 0.00 (4H, s), 0.84 (4H, d), 2.86 (3H, dd), 2.94 (3H, d), 3.39 (3H, d), 4.69 (2H, d), 7.14-7.27 (3H, m), 7.29-7.47 (4H, m), 7.54 (1H, td), 7.75 (1H, d), 8.14 (1H, td), 8.19 (1H, d), 8.73-8.82 (1H, m), 9.19 (1H, s), 11.00 (1H, d), 11.36 (1H, d) Check? |
| 86 | 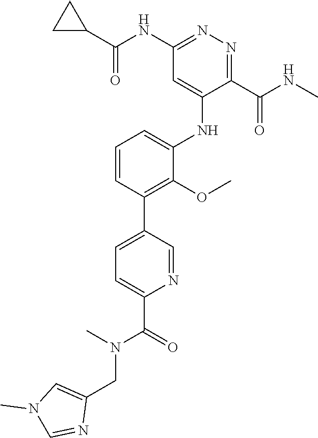 | 570.4 [M + H]+ | ¹H NMR (DMSO-d6, 400 MHz) 0.81-0.87 (4H, m), 2.06-2.14 (1H, m), 2.86 (3H, d), 2.98 (3H, d), 3.40 (3H, s), 3.62 (3H, d), 4.49 (2H, d), 7.03 (1H, d), 7.31-7.39 (2H, m), 7.48-7.56 (1H, m), 7.67 (1H, d), 7.74 (1H, d), 8.12 (1H, dt), 8.20 (1H, s), 8.78 (1H, d), 9.19 (1H, d), 11.00 (1H, s), 11.36 (1H, s) |
| 93 | 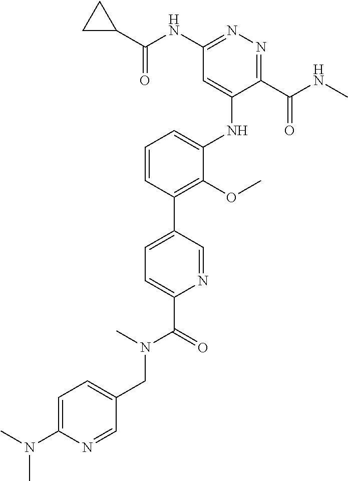 | 610.3 [M + H]+ | ¹H NMR (400 MHz, DMSO- d6) 11.36 (s, 1H), 1.00 (d, J = 3.9 Hz, 1H), 9.23-9.14 (m, 1H), 8.82-8.74 (m, 1H), 8.24- 7.93 (m, 3H), 7.72 (t, J = 8.8 Hz, 1H), 7.57-7.48 (m, 2H), 7.39-7.28 (m, 2H), 6.71-6.60 (m, 1H), 4.52 (d, J = 40.9 Hz, 2H), 3.39 (d, J = 2.4 Hz, 3H), 3.01 (d, J = 10.9 Hz, 6H), 2.90 (s, 3H), 2.86 (d, J = 4.7 Hz, 3H), 2.13-2.04 (m, 1H), 0.87-0.81 (m, 4H) |
| 94 | 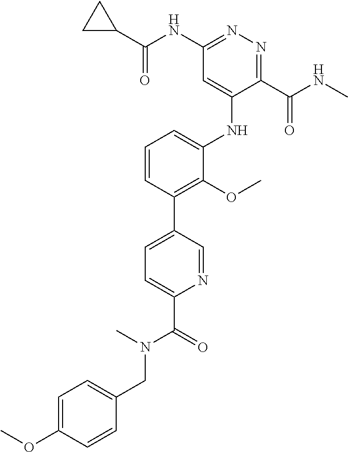 | 596.3 [M + H]+ | ¹H NMR (400 MHz, DMSO- d6) 11.36 (s, 1H), 11.00 (d, J = 7.0 Hz, 1H), 9.22-9.14 (m, 1H), 8.78 (t, J = 2.7 Hz, 1H), 8.19 (d, J = 6.4 Hz, 1H), 8.17-8.09 (m, 1H), 7.72 (d, J = 8.1 Hz, 1H), 7.57-7.48 (m, 1H), 7.39-7.28 (m, 3H), 7.23 (d, J = 8.2 Hz, 1H), 7.00-6.88 (m, 2H), 4.61 (d, J = 39.5 Hz, 2H), 3.75 (d, J = 11.7 Hz, 3H), 3.39 (d, J = 9.2 Hz, 3H), 2.98-2.75 (m, 6H), 2.09 (q, J = 6.4 Hz, 1H), 0.87-0.80 (m, 4H). |
| 96 | 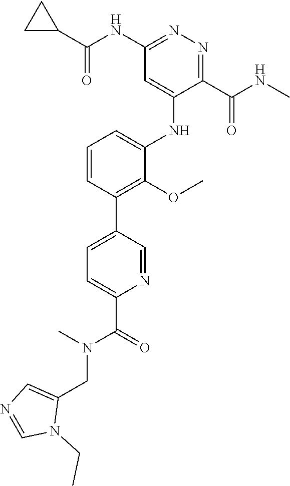 [SMILES: CCn1cncc1CN(C)C(=O)c1ccc(-c2cccc(Nc3cc(NC(=O)C4CC4)nnc3C(=O)NC)c2OC)cn1] | 584.3 [M + H]+ | ¹H NMR (400 MHz, DMSO- d6) 11.36 (s, 1H), 11.00 (s, 1H), 9.19 (q, J = 4.7 Hz, 1H), 8.82-8.75 (m, 1H), 8.19 (s, 1H), 8.17-8.11 (m, 1H), 7.77- 7.67 (m, 2H), 7.57-7.50 (m, 1H), 7.39-7.28 (m, 2H), 6.89 (d, J = 60.5 Hz, 1H), 4.76 (d, J = 3.1 Hz, 2H), 4.08-3.78 (m, 2H), 3.39 (d, J = 5.8 Hz, 3H), 2.96-2.80 (m, 6H), 2.15- 2.04 (m, 1H), 1.38-1.07 (m, 3H), 0.83 (q, J = 4.0 Hz, 4H). |
| 99 | 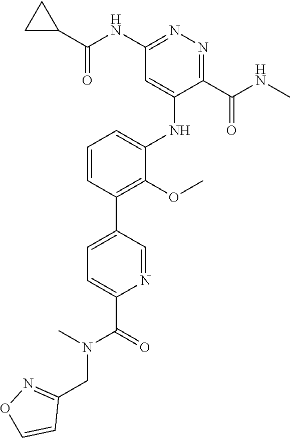 [SMILES: CNC(=O)c1nnc(NC(=O)C2CC2)cc1Nc1cccc(-c2ccc(C(=O)N(C)Cc3ccon3)nc2)c1OC] | 557.2 [M + H]+ | ¹H NMR (400 MHz, DMSO- d6) 11.36 (s, 1H), 11.00 (d, J = 3.6 Hz, 1H), 9.18 (d, J = 4.9 Hz, 1H), 8.96-8.87 (m, 1H), 8.82-8.75 (m, 1H), 8.19 (d, J = 4.6 Hz, 1H), 8.19-8.11 (m, 1H), 7.79-7.71 (m, 1H), 7.58-7.50 (m, 1H), 7.39-7.29 (m, 2H), 6.66-6.57 (m, 1H), 4.81 (s, 2H), 3.39 (d, J = 9.1 Hz, 3H), 3.04 (d, J = 11.4 Hz, 3H), 2.86 (d, J = 4.8 Hz, 3H), 2.14-2.06 (m, 1H), 0.84 (q, J = 3.4, 2.9 Hz, 4H). |
| 100 | 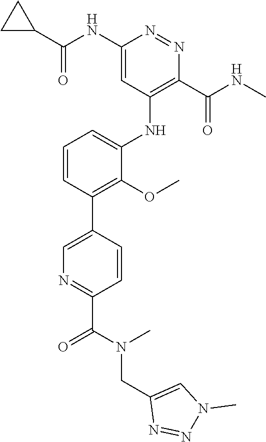 | 571.2 [M + H]+ | |
| 101 | 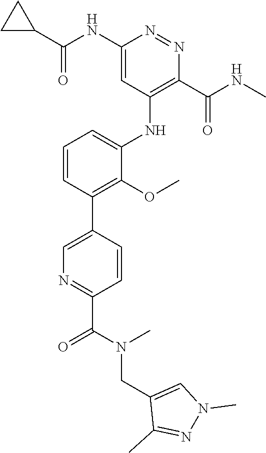 | 584.2 [M + H]+ | |
| 102 | 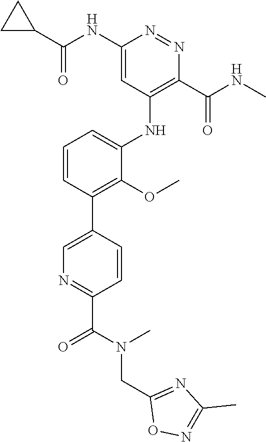 [SMILES: CNC(=O)c1nnc(NC(=O)C2CC2)cc1Nc1cccc(-c2ccc(C(=O)N(C)Cc3nc(C)no3)nc2)c1OC] | 572.1 [M + H]+ | |
| 108 | 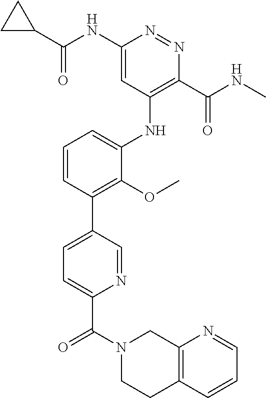 [SMILES: CNC(=O)c1nnc(NC(=O)C2CC2)cc1Nc1cccc(-c2ccc(C(=O)N3CCc4cccnc4C3)nc2)c1OC] | 579.4 [M + H]+ | |
| 113 | 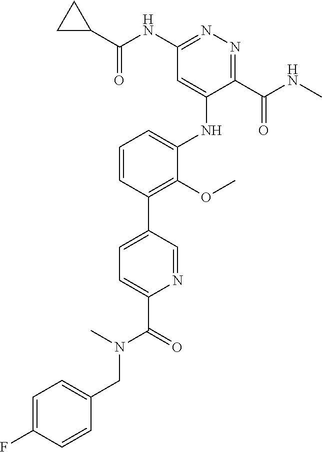 [SMILES: CNC(=O)c1nnc(NC(=O)C2CC2)cc1Nc1cccc(-c2ccc(C(=O)N(C)Cc3ccc(F)cc3)nc2)c1OC] | 584.5 [M + H] | ¹H NMR (400 MHz, DMSO- d6) 11.35 (d, J = 2.7 Hz, 1H), 10.99 (d, J = 7.9 Hz, 1H), 9.17 (s, 1H), 8.78 (dd, J = 13.4, 2.2 Hz, 1H), 8.22-8.10 (m, 2H), 7.74 (dd, J = 8.1, 4.8 Hz, 1H), 7.54 (td, J = 6.9, 2.8 Hz, 1H), 7.46-7.29 (m, 4H), 7.21 (dt, J = 19.1, 8.8 Hz, 2H), 4.72 (s, 1H), 4.64 (s, 1H), 3.39 (d, J = 12.3 Hz, 3H), 2.94 (d, J = 9.3 Hz, 3H), 2.86 (dd, J = 4.8, 1.4 Hz, 3H), 2.09 (d, J = 6.8 Hz, 1H), 0.84 (d, J = 5.0 Hz, 4H). |
| 114 | 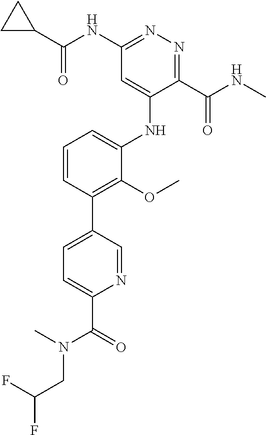 [SMILES: CNC(=O)c1nnc(NC(=O)C2CC2)cc1Nc1cccc(-c2ccc(C(=O)N(C)CC(F)F)nc2)c1OC] | 540.2 [M + H] | ¹H NMR (400 MHz, DMSO- d6) 11.35 (s, 1H), 11.00 (d, J = 2.9 Hz, 1H), 9.18 (d, J = 5.0 Hz, 1H), 8.79 (dd, J = 10.4, 2.2 Hz, 1H), 8.22-8.12 (m, 2H), 7.75 (dd, J = 21.2, 8.1 Hz, 1H), 7.54 (dd, J = 5.8, 2.8 Hz, 1H), 7.40-7.31 (m, 2H), 6.34 (td, J = 55.9, 21.5 Hz, 1H), 4.08-3.89 (m, 2H), 3.40 (d, J = 5.3 Hz, 3H), 3.13 (s, 3H), 2.87 (d, J = 4.8 Hz, 3H), 2.14 ^{••}C 2.06 (m, 1H), 0.88-0.81 (m, 4H) |
| 115* | 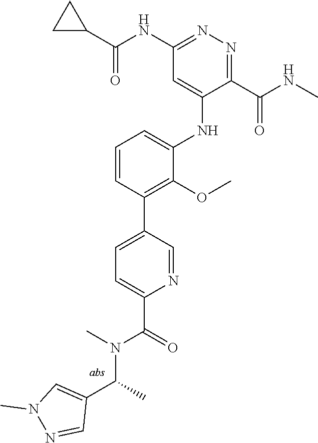 | 584.5 [M + H] | ¹H NMR (400 MHz, DMSO- d6) 11.36 (s, 1H), 11.00 (d, J = 5.2 Hz, 1H), 9.19 (q, J = 4.8 Hz, 1H), 8.79 (ddd, J = 13.4, 2.2, 0.9 Hz, 1H), 8.20 (d, J = 3.9 Hz, 1H), 8.14 (ddd, J = 8.0, 5.6, 2.2 Hz, 1H), 7.76-7.66 (m, 2H), 7.57- 7.50 (m, 1H), 7.40-7.28 (m, 3H), 5.02 (q, J = 6.8 Hz, 1H), 3.82 (d, J = 9.7 Hz, 3H), 3.40 (s, 3H), 2.86 (d, J = 4.8 Hz, 3H), 2.76-2.70 (s, 3H), 2.15- 2.04 (m, 1H), 1.48 (t, J = 7.0 Hz, 3H), 0.88-0.81 (m, 4H). |
| 116* | 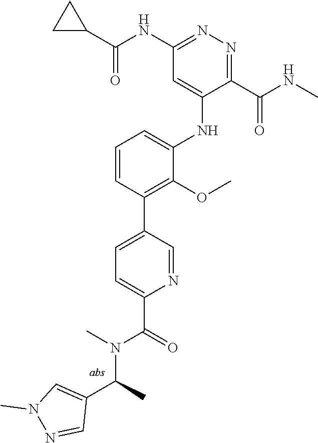 | 584.5 [M + H] | ¹H NMR (400 MHz, DMSO- d6) 11.36 (s, 1H), 11.00 (d, J = 5.2 Hz, 1H), 9.19 (q, J = 4.8 Hz, 1H), 8.79 (ddd, J = 13.4, 2.2, 0.9 Hz, 1H), 8.20 (d, J = 3.9 Hz, 1H), 8.14 (ddd, J = 8.0, 5.6, 2.2 Hz, 1H), 7.76-7.66 (m, 2H), 7.57- 7.50 (m, 1H), 7.40-7.28 (m, 3H), 5.02 (q, J = 6.8 Hz, 1H), 3.82 (d, J = 9.7 Hz, 3H), 3.40 (s, 3H), 2.86 (d, J = 4.8 Hz, 3H), 2.76-2.70 (s, 3H), 2.15- 2.04 (m, 1H), 1.48 (t, J = 7.0 Hz, 3H), 0.88-0.81 (m, 4H). |
| 120 | 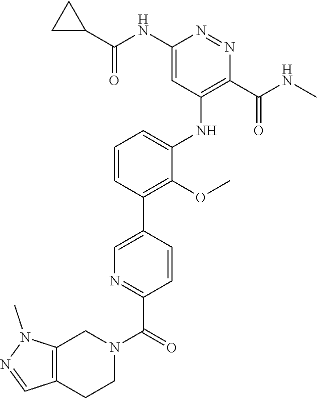 [SMILES: CNC(=O)c1nnc(NC(=O)C2CC2)cc1Nc1cccc(-c2ccc(C(=O)N3CCc4cnn(C)c4C3)nc2)c1OC] | 582.5 [M + H] | |
| 133 | 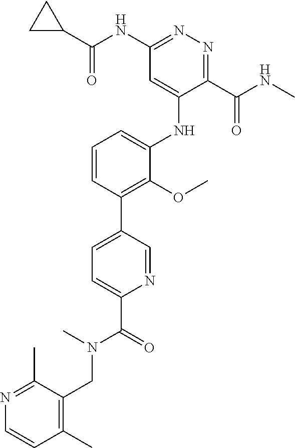 [SMILES: CNC(=O)c1nnc(NC(=O)C2CC2)cc1Nc1cccc(-c2ccc(C(=O)N(C)Cc3c(C)ccnc3C)nc2)c1OC] | 594.7 [M + H] | ¹H NMR (400 MHz, DMSO- d6): 11.35 (s, 1H), 11.00 (s, 1H), 9.18 (d, J = 4.8 Hz, 1H), 8.84-8.74 (m, 1H), 8.24 (dd, J = 20.6, 4.9 Hz, 1H), 8.19 (s, 1H), 8.14 (dd, J = 8.1, 2.2 Hz, 1H), 7.70 (d, J = 8.1 Hz, 1H), 7.54 (dd, J = 7.3, 2.2 Hz, 1H), 7.33 (dd, J = 6.7, 2.6 Hz, 2H), 7.13 (d, J = 5.0 Hz, 1H), 4.86 (s, 2H), 4.78 (s, 0H), 2.86 (d, J = 4.8 Hz, 3H), 2.71 (d, J = 13.4 Hz, 3H), 2.56 (s, 3H), 2.40 (d, J = 14.0 Hz, 3H), 2.26 (s, 1H), 2.09 (q, J = 6.2 Hz, 1H), 0.87- 0.81 (m, 5H) |
| 134 | 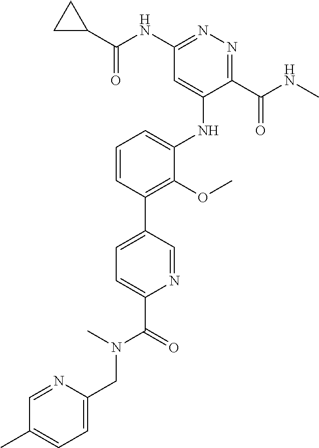 | 581.5 [M + H] | ¹H NMR (400 MHz, DMSO- d6) 11.35 (s, 1H), 10.99 (d, J = 12.3 Hz, 1H), 9.19 (t, J = 4.6 Hz, 1H), 8.83-8.78 (m, 1H), 8.74-8.69 (m, 1H), 8.41- 8.35 (m, 2H), 8.08-7.62 (m, 3H), 7.40-7.25 (m, 3H), 4.75 (m, 2H), 3.05 (s, 3H), 2.99 (s, 3H), 2.86 (m, 3H), 2.29 (m, 3H), 2.08 (m, 1H), 0.83 (m, 4H). |
| 135 | 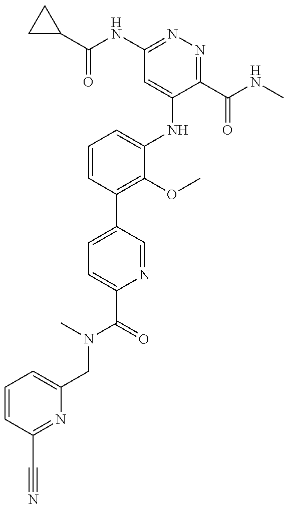 [SMILES: CNC(=O)c1nnc(NC(=O)C2CC2)cc1Nc1cccc(-c2ccc(C(=O)N(C)Cc3cccc(C#N)n3)nc2)c1OC] | 592.5 [M + H] | ¹H NMR (400 MHz, DMSO- d6) 11.36 (d, J = 5.3 Hz, 1H), 10.99 (d, J = 11.9 Hz, 1H), 9.18 (t, J = 4.8 Hz, 1H), 8.82 (dd, J = 2.2, 0.9 Hz, 1H), 8.66-7.95 (m, 4H), 7.80-7.68 (m, 2H), 7.53 (ddd, J = 12.6, 7.2, 2.4 Hz, 1H), 7.40-7.25 (m, 2H), 4.88 (d, J = 7.1 Hz, 2H), 3.41 (s, 3H), 3.06 (s, 3H), 2.86 (dd, J = 4.8, 2.8 Hz, 3H), 2.09 (tt, J = 7.7, 3.9 Hz, 1H), 0.88-0.78 (m, 4H). |
| 136 | 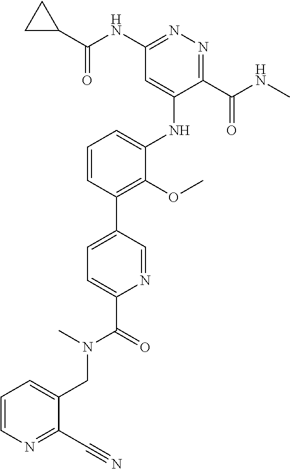 [SMILES: CNC(=O)c1nnc(NC(=O)C2CC2)cc1Nc1cccc(-c2ccc(C(=O)N(C)Cc3cccnc3C#N)nc2)c1OC] | 592.5 [M + H] | ¹H NMR (Chloroform-d, 400 MHz) 0.91-1.02 (2H, m), 1.08-1.19 (2H, m), 1.76 (1H, dd), 3.07 (3H, dd), 3.21 (3H, d), 3.47 (3H, d), 5.11 (2H, d), 7.20 (1H, ddd), 7.28-7.37 (1H, m), 7.50-7.66 (2H,m), 7.90 (1H, dd), 8.07-8.32 (4H, m), 8.64-8.89 (2H, m), 9.11 (1H, s), 11.18 (1H, d) |
| 138* | 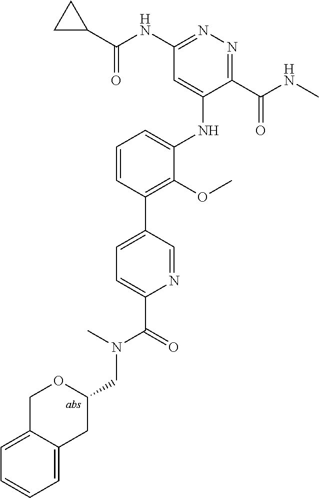 [SMILES: CNC(=O)c1nnc(NC(=O)C2CC2)cc1Nc1cccc(-c2ccc(C(=O)N(C)C[C@@H]3Cc4ccccc4CO3)nc2)c1OC] | 622.3 [M + H] | ¹H NMR (400 MHz, DMSO- d6) 11.36 (d, J = 4.0 Hz, 1H), 11.00 (d, J = 9.4 Hz, 1H), 9.19 (t, J = 4.4 Hz, 1H), 8.82- 8.66 (m, 1H), 8.20 (d, J = 7.7 Hz, 1H), 8.13 (ddd, J = 9.9, 8.1, 2.2 Hz, 1H), 7.70 (dd, J = 8.0, 5.2 Hz, 1H), 7.54 (td, J = 7.0, 3.2 Hz, 1H), 7.40-7.29 (m, 2H), 7.21-6.98 (m, 4H), 4.92-4.65 (m, 2H), 4.09-3.95 (m, 1H), 3.82-3.54 (m, 2H), 3.39 (d, J = 16.8 Hz, 3H), 3.14 (d, J = 11.2 Hz, 3H), 2.86 (dd, J = 4.9, 2.5 Hz, 3H), 2.84-2.70 (m, 1H), 2.61- 2.53 (m, 1H), 2.10 (q, J = 5.6 Hz, 1H), 0.84 (t, J = 7.0 Hz, 4H). |
| 139* | 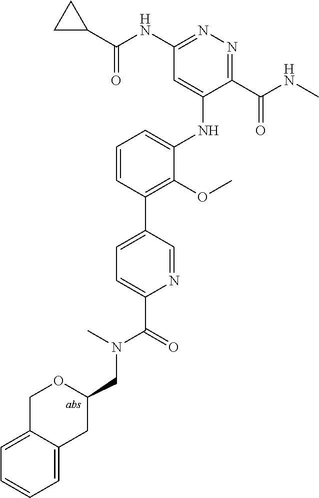 [SMILES: CNC(=O)c1nnc(NC(=O)C2CC2)cc1Nc1cccc(-c2ccc(C(=O)N(C)C[C@H]3Cc4ccccc4CO3)nc2)c1OC] | 622.3 [M + H] | ¹H NMR (400 MHz, DMSO- d6) 11.36 (d, J = 4.0 Hz, 1H), 11.00 (d, J = 9.4 Hz, 1H), 9.19 (t, J = 4.4 Hz, 1H), 8.82- 8.66 (m, 1H), 8.20 (d, J = 7.7 Hz, 1H), 8.13 (ddd, J = 9.9, 8.1, 2.2 Hz, 1H), 7.70 (dd, J = 8.0, 5.2 Hz, 1H), 7.54 (td, J = 7.0, 3.2 Hz, 1H), 7.40-7.29 (m, 2H), 7.21-6.98 (m, 4H), 4.92-4.65 (m, 2H), 4.09-3.95 (m, 1H), 3.82-3.54 (m, 2H), 3.39 (d, J = 16.8 Hz, 3H), 3.14 (d, J = 11.2 Hz, 3H), 2.86 (dd, J = 4.9, 2.5 Hz, 3H), 2.84-2.70 (m, 1H), 2.61- 2.53 (m, 1H), 2.10 (q, J = 5.6 Hz, 1H), 0.84 (t, J = 7.0 Hz, 4H) |
| 140* | 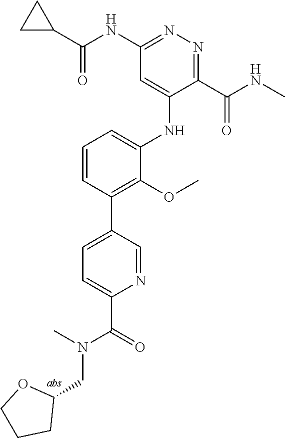 [SMILES: CNC(=O)c1nnc(NC(=O)C2CC2)cc1Nc1cccc(-c2ccc(C(=O)N(C)C[C@@H]3CCCO3)nc2)c1OC] | 560.3 [M + H] | ¹H NMR (400 MHz, DMSO- d6) 11.36 (s, 1H), 11.00 (s, 1H), 9.19 (d, J = 4.9 Hz, 1H), 8.80-8.71 (m, 1H), 8.20 (d, J = 2.1 Hz, 1H), 8.12 (dt, J = 8.2, 2.9 Hz, 1H), 7.64 (dd, J = 8.1, 3.1 Hz, 1H), 7.54 (d, J = 7.2 Hz, 1H), 7.39-7.29 (m, 2H), 4.17-4.01 (m, 1H), 3.91- 3.49 (m, 3H), 3.50-3.35 (m, 4H), 3.06 (d, J = 6.7 Hz, 3H), 2.86 (d, J = 4.8 Hz, 3H), 2.09 (q, J = 6.2 Hz, 1H), 2.00-1.68 (m, 2H), 1.68-1.31 (m, 2H), 0.88-0.81 (m, 4H). |
| 146 | 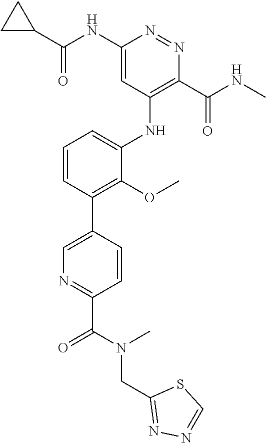 [SMILES: CNC(=O)c1nnc(NC(=O)C2CC2)cc1Nc1cccc(-c2ccc(C(=O)N(C)Cc3nncs3)nc2)c1OC] | 574.0 [M + H] | |
| 149 | 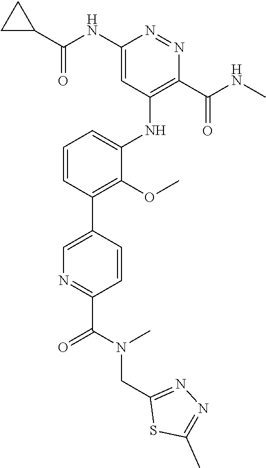 [SMILES: CNC(=O)c1nnc(NC(=O)C2CC2)cc1Nc1cccc(-c2ccc(C(=O)N(C)Cc3nnc(C)s3)nc2)c1OC] | 588.4 [M + H] | |
| 154 | 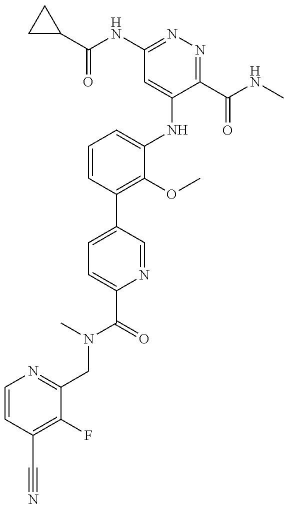 [SMILES: CNC(=O)c1nnc(NC(=O)C2CC2)cc1Nc1cccc(-c2ccc(C(=O)N(C)Cc3nccc(C#N)c3F)nc2)c1OC] | 609.6 [M + H] | ¹H NMR (400 MHz, DMSO- d6) \|Ä 11.37 (d, J = 3.1 Hz, 2H), 11.01 (d, J = 7.9 Hz, 2H), 9.21 ^{••}C 9.14 (m, 2H), 8.81 (d, J = 2.2 Hz, 1H), 8.71-8.60 (m, 3H), 8.15 (dd, J = 9.4, 4.3 Hz, 3H), 8.08 (dd, J = 8.1, 2.3 Hz, 1H), 7.94 (dt, J = 24.6, 4.8 Hz, 2H), 7.69 (dd, J = 10.9, 8.1 Hz, 2H), 7.54 (td, J = 7.5, 2.2 Hz, 2H), 7.40-7.25 (m, 4H), 5.08 (s, 2H), 4.97 (d, J = 1.8 Hz, 2H), 3.39 (d, J = 17.1 Hz, 18H), 3.13 (d, J = 17.9 Hz, 5H), 2.86 (d, J = 4.7 Hz, 5H), 2.13 ^{••}C 2.04 (m, 2H), 1.26 (dd, J = 10.9, 4.8 Hz, 2H), 0.84 (q, J = 4.7, 4.0 Hz, 6H) |
| 158 | 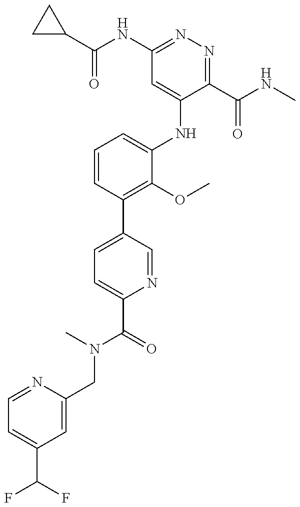 [SMILES: CNC(=O)c1nnc(NC(=O)C2CC2)cc1Nc1cccc(-c2ccc(C(=O)N(C)Cc3cc(C(F)F)ccn3)nc2)c1OC] | 617.3 [M + H] | ¹H NMR (400 MHz, DMSO- d6) 11.35 (d, J = 5.4 Hz, 1H), 11.00 (d, J = 11.8 Hz, 1H), 9.18 (p, J = 4.6 Hz, 1H), 8.84-8.67 (m, 2H), 8.23-8.03 (m, 2H), 7.73 (ddd, J = 17.9, 8.1, 0.8 Hz, 1H), 7.58-7.44 (m, 3H), 7.38-7.22 (m, 2H), 7.06 (dd, J = 55.1, 21.7 Hz, 1H), 4.90 (d, J = 8.5 Hz, 2H), 3.42 (s, 2H), 3.08 (d, J = 33.0 Hz, 3H), 2.86 (dd, J = 4.9, 3.0 Hz, 3H), 2.16-2.04 (m, 1H), 0.88-0.78 (m, 4H) |
| 160* | 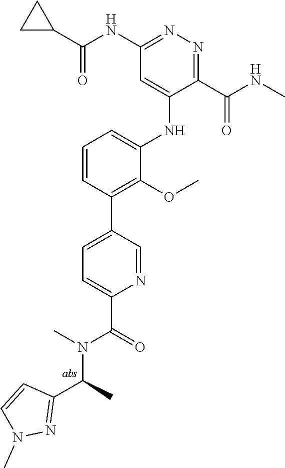 [SMILES: CNC(=O)c1nnc(NC(=O)C2CC2)cc1Nc1cccc(-c2ccc(C(=O)N(C)[C@@H](C)c3ccn(C)n3)nc2)c1OC] | 584.3 [M + H] | ¹H NMR (400 MHz, DMSO- d6) 11.35 (s, 1H), 10.99 (d, J = 7.6 Hz, 1H), 9.18 (d, J = 5.1 Hz, 1H), 8.78 (dd, J = 6.6, 2.2 Hz, 1H), 8.19 (d, J = 5.1 Hz, 1H), 8.13 (dt, J = 8.1, 2.3 Hz, 1H), 7.74-7.60 (m, 2H), 7.57-7.49 (m, 1H), 7.33 (dd, J = 4.9, 3.5 Hz, 2H), 6.20 (dd, J = 10.6, 2.2 Hz, 1H), 5.10 (q, J = 6.9 Hz, 1H), 3.81 (d, J = 16.2 Hz, 3H), 3.40 (s, 3H), 2.86 (d, J = 4.8 Hz, 3H), 2.77 (s, 3H), 2.09 (m, J = 6.0, 5.5 Hz, 1H), 1.53 (t, J = 7.0 Hz, 3H), 0.90-0.81 (m, 4H). |
| 161* | 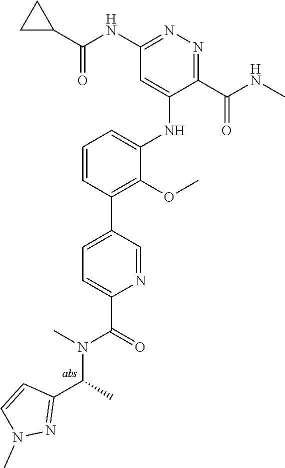 [SMILES: CNC(=O)c1nnc(NC(=O)C2CC2)cc1Nc1cccc(-c2ccc(C(=O)N(C)[C@H](C)c3ccn(C)n3)nc2)c1OC] | 584.3 [M + H]+ | ¹H NMR (400 MHz, DMSO- d6) 11.35 (s, 1H), 10.99 (d, J = 7.6 Hz, 1H), 9.18 (d, J = 5.1 Hz, 1H), 8.78 (dd, J = 6.6, 2.2 Hz, 1H), 8.19 (d, J = 5.1 Hz, 1H), 8.13 (dt, J = 8.1, 2.3 Hz, 1H), 7.74-7.60 (m, 2H), 7.57-7.49 (m, 1H), 7.33 (dd, J = 4.9, 3.5 Hz, 2H), 6.20 (dd, J = 10.6, 2.2 Hz, 1H), 5.10 (q, J = 6.9 Hz, 1H), 3.81 (d, J = 16.2 Hz, 3H), 3.40 (s, 3H), 2.86 (d, J = 4.8 Hz, 3H), 2.77 (s, 3H), 2.09 (m, J = 6.0, 5.5 Hz, 1H), 1.53 (t, J = 7.0 Hz, 3H), 0.90-0.81 (m, 4H). |
| 166 | 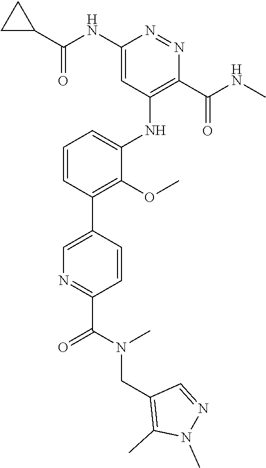 [SMILES: CNC(=O)c1nnc(NC(=O)C2CC2)cc1Nc1cccc(-c2ccc(C(=O)N(C)Cc3cnn(C)c3C)nc2)c1OC] | 584.1 [M + H]+ | ¹H NMR (500 MHz, DMSO) δ 11.33 (s, 1H), 10.99 (d, J = 1.9 Hz, 1H), 9.16 (q, J = 4.8 Hz, 1H), 8.78 (dd, J = 31.9, 2.2 Hz, 1H), 8.18 (d, J = 2.0 Hz, 1H), 8.12 (ddd, J = 13.1, 8.0, 2.3 Hz, 1H), 7.67 (dd, J = 17.4, 8.1 Hz, 1H), 7.53 (dt, J = 7.6, 2.6 Hz, 1H), 7.41- 7.25 (m, 3H), 4.47 (d, J = 34.0 Hz, 2H), 3.69 (d, J = 31.5 Hz, 3H), 3.39 (d, J = 3.1 Hz, 3H), 2.90 (d, J = 23.5 Hz, 3H), 2.86 (d, J = 4.8 Hz, 3H), 2.17 (d, J = 126.5 Hz, 3H), 2.10 (d, J = 6.9 Hz, 1H), 1.09- 0.06 (m, 4H). |
| 167 | 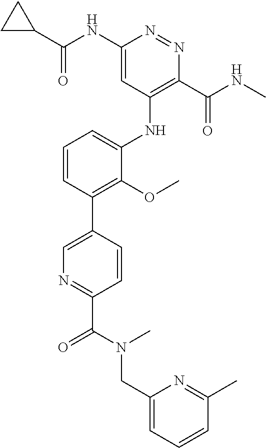 [SMILES: CNC(=O)c1nnc(NC(=O)C2CC2)cc1Nc1cccc(-c2ccc(C(=O)N(C)Cc3cccc(C)n3)nc2)c1OC] | 581.1 [M + H]+ | ¹H NMR (500 MHz, DMSO) δ 11.34 (d, J = 6.0 Hz, 1H), 10.98 (d, J = 14.1 Hz, 1H), 9.16 (t, J = 5.1 Hz, 1H), 8.75 (dd, J = 64.7, 2.2 Hz, 1H), 8.28-8.04 (m, 2H), 7.89 (d, J = 50.8 Hz, 1H), 7.76 (dd, J = 8.1, 5.6 Hz, 1H), 7.53 (ddd, J = 14.1, 7.5, 2.1 Hz, 1H), 7.44-7.22 (m, 4H), 4.82 (d, J = 17.3 Hz, 2H), 3.38 (d, J = 28.2 Hz, 3H), 3.07 (d, J = 40.1 Hz, 3H), 2.86 (dd, J = 4.8, 3.5 Hz, 3H), 2.57 (s, 3H), 2.14-1.97 (m, 1H), 0.83 (td, J = 7.6, 7.0, 4.6 Hz, 4H). |
| 168 | 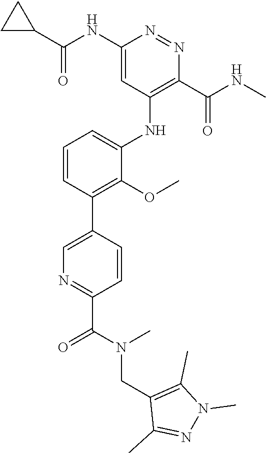 [SMILES: CNC(=O)c1nnc(NC(=O)C2CC2)cc1Nc1cccc(-c2ccc(C(=O)N(C)Cc3c(C)nn(C)c3C)nc2)c1OC] | 598.0 [M + H]+ | ¹H NMR (400 MHz, DMSO) δ 11.35 (s, 1H), 10.99 (s, 1H), 9.17 (d, J = 4.9 Hz, 1H), 8.92- 8.69 (m, 1H), 8.25-8.07 (m, 2H), 7.76-7.60 (m, 1H), 7.53 (dd, J = 7.3, 2.2 Hz, 1H), 7.44-7.24 (m, 2H), 4.50 (d, J = 18.9 Hz, 2H), 3.67-3.53 (m, 3H), 3.39 (s, 3H), 2.86 (d, J = 4.8 Hz, 3H), 2.79 (d, J = 19.2 Hz, 3H), 2.19 (d, J = 46.1 Hz, 3H), 2.10 (s, 1H), 2.09-1.85 (m, 3H), 0.83 (d, J = 7.9 Hz, 4H). |
| 174 | 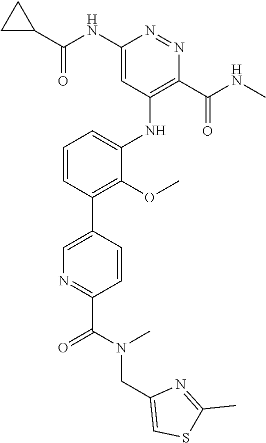 [SMILES: CNC(=O)c1nnc(NC(=O)C2CC2)cc1Nc1cccc(-c2ccc(C(=O)N(C)Cc3csc(C)n3)nc2)c1OC] | 587.5 [M + H]+ | ¹H NMR (500 MHz, DMSO) δ 11.32 (d, J = 2.1 Hz, 1H), 10.98 (d, J = 6.3 Hz, 1H), 9.31-9.07 (m, 1H), 8.76 (dd, J = 13.0, 2.2 Hz, 1H), 8.17 (d, J = 6.5 Hz, 1H), 8.14- 8.01 (m, 1H), 7.70 (dd, J = 9.2, 8.1 Hz, 1H), 7.52 (td, J = 6.7, 2.7 Hz, 1H), 7.43-7.22 (m, 3H), 4.71 (d, J = 20.1 Hz, 2H), 3.02 (d, J = 17.1 Hz, 3H), 2.85 (d, J = 4.7 Hz, 3H), 2.62 (d, J = 31.5 Hz, 3H), 2.53 (s, 3H), 2.08 (s, J = 3.4 Hz, 1H), 0.96-0.60 (m, 4H). |
| 178 | 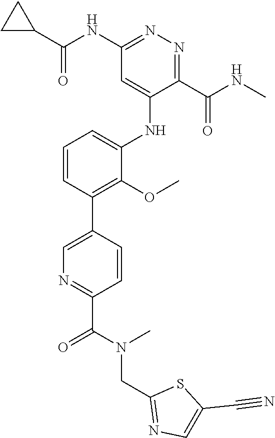 [SMILES: CNC(=O)c1nnc(NC(=O)C2CC2)cc1Nc1cccc(-c2ccc(C(=O)N(C)Cc3ncc(C#N)s3)nc2)c1OC] | 598.2 [M + H]+ | ¹H NMR (40 0MHz, DMSO- d6) 11.37 (s, 1H), 11.00 (d, J = 3.2 Hz, 1H), 9.19 (s, 1H), 8.81 (d, J = 5.9 Hz, 1H), 8.66 (d, J = 9.9 Hz, 1H), 8.18 (t, J = 7.8 Hz, 2H), 7.83 (dd, J = 41.5, 8.2 Hz, 1H), 7.55 (d, J = 4.6 Hz, 1H), 7.38-7.32 (m, 2H), 5.09 (d, J = 10.4 Hz, 2H), 3.40 (d, J = 7.2 Hz, 3H), 3.13 (d, J = 45.9 Hz, 3H), 2.86 (d, J = 4.8 Hz, 3H), 2.10 (s, 1H), 0.84 (d, J = 6.6 Hz, 4H). |
| 179 | 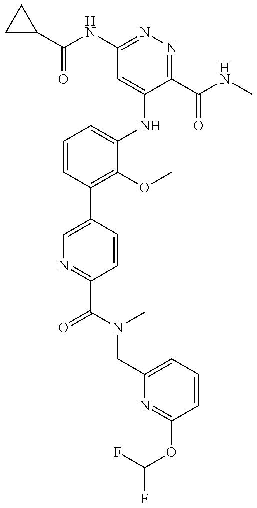 [SMILES: CNC(=O)c1nnc(NC(=O)C2CC2)cc1Nc1cccc(-c2ccc(C(=O)N(C)Cc3cccc(OC(F)F)n3)nc2)c1OC] | 633.3 [M + H]+ | ¹H NMR (400 MHz, DMSO- d6) 11.35 (d, J = 4.8 Hz, 1H), 10.99 (d, J = 9.7 Hz, 1H), 9.22-9.15 (m, 1H), 8.84-8.69 (m, 1H), 8.22-8.07 (m, 2H), 8.01-7.85 (m, 1H), 7.79-7.49 (m, 3H), 7.41-7.28 (m, 2H), 7.22 (dd, J = 33.7, 7.5 Hz, 1H), 6.99 (dd, J = 19.8, 8.1 Hz, 1H), 4.78 (d, J = 9.4 Hz, 2H), 3.41 (s, 3H), 3.08 (d, J = 25.7 Hz, 3H), 2.86 (dd, J = 4.9, 2.7 Hz, 3H), 2.10 (s, 1H), 0.86-0.81 (m, 4H). |
| 181* | 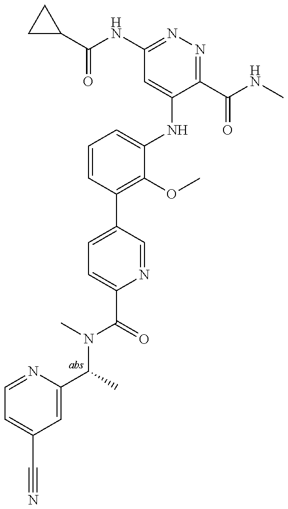 [SMILES: CNC(=O)c1nnc(NC(=O)C2CC2)cc1Nc1cccc(-c2ccc(C(=O)N(C)[C@H](C)c3cc(C#N)ccn3)nc2)c1OC] | 606.3 [M + H]+ | ¹H NMR (400 MHz, DMSO- d6) 11.35 (d, J = 2.2 Hz, 1H), 11.00 (d, J = 8.9 Hz, 1H), 9.21-9.15 (m, 1H), 8.81 (m, 2H), 8.22-8.10 (m, 2H), 7.90 (d, J = 3.0 Hz, 1H), 7.85-7.78 (m, 1H), 7.76 (dd, J = 8.1, 5.3 Hz, 1H), 7.54 (td, J = 6.7, 3.1 Hz, 1H), 7.39-7.29 (m, 2H), 5.90-5.38 (m, 1H), 3.40 (d, J = 4.0 Hz, 3H), 2.91-2.83 (m, 3H), 2.80-2.04 (m, 3H), 2.10 (m, 1H), 1.65 (d, J = 7.0 Hz, 3H), 0.87-0.80 (m, 4H). |
| 188 | 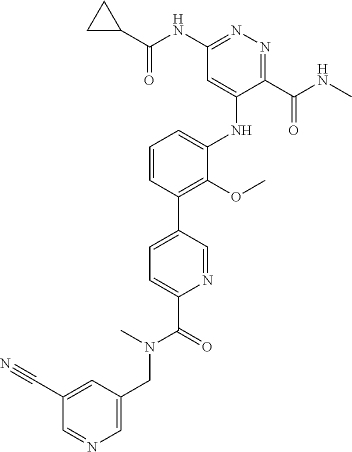 [SMILES: CNC(=O)c1nnc(NC(=O)C2CC2)cc1Nc1cccc(-c2ccc(C(=O)N(C)Cc3cncc(C#N)c3)nc2)c1OC] | 592.3 [M + H]+ | ¹H NMR (400 MHz, DMSO- d6) 11.35 (d, J = 4.1 Hz, 1H), 10.99 (d, J = 8.3 Hz, 1H), 9.18 (t, J = 4.5 Hz, 1H), 8.98 (dd, J = 10.6, 2.0 Hz, 1H), 8.90 (d, J = 2.1 Hz, 1H), 8.85-8.78 (m, 1H), 8.31 (m, 1H), 8.22-8.09 (m, 2H), 7.84- 7.76 (m, 1H), 7.54 (m, 1H), 7.39-7.28 (m, 2H), 4.79 (d, J = 17.1 Hz, 2H), 3.39 (d, J = 12.2 Hz, 3H), 3.06 (s, 2H), 3.00 (s, 1H), 2.86 (dd, J = 4.9, 1.6 Hz, 3H), 2.14-2.05 (m, 1H), 0.88-0.78 (m, 4H). |
| 193* | 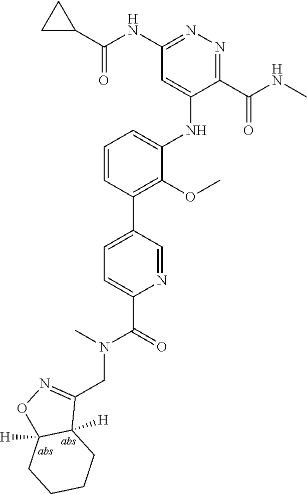 [SMILES: CNC(=O)c1nnc(NC(=O)C2CC2)cc1Nc1cccc(-c2ccc(C(=O)N(C)CC3=NO[C@@H]4CCCC[C@H]34)nc2)c1OC] | 613.3 [M + H]+ | ¹H NMR (400 MHz, DMSO- d6) 11.35 (s, 1H), 10.99 (d, J = 2.7 Hz, 1H), 9.17 (q, J = 4.8 Hz, 1H), 8.82-8.74 (m, 1H), 8.21-8.11 (m, 2H), 7.75- 7.67 (m, 1H), 7.58-7.51 (m, 1H), 7.39-7.26 (m, 2H), 4.69- 4.34 (m, 2H), 4.34-4.24 (m, 1H), 3.40 (d, J = 8.0 Hz, 3H), 3.03 (d, J = 11.2 Hz, 3H), 2.86 (d, J = 4.8 Hz, 3H), 2.13-2.06 (m, 1H), 1.94-0.89 (m, 9H), 0.88-0.81 (m, 4H). |
| 194* | 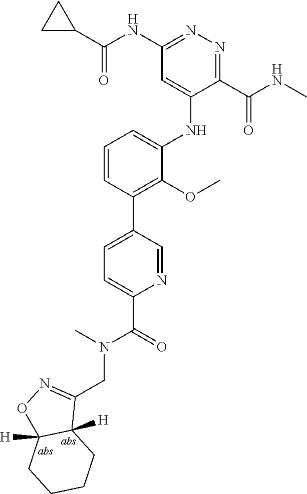 [SMILES: CNC(=O)c1nnc(NC(=O)C2CC2)cc1Nc1cccc(-c2ccc(C(=O)N(C)CC3=NO[C@H]4CCCC[C@@H]34)nc2)c1OC] | 613.3 [M + H]+ | ¹H NMR (400 MHz, DMSO- d6) 11.35 (s, 1H), 10.99 (d, J = 2.7 Hz, 1H), 9.17 (q, J = 4.8 Hz, 1H), 8.82-8.74 (m, 1H), 8.21-8.11 (m, 2H), 7.75- 7.67 (m, 1H), 7.58-7.51 (m, 1H), 7.39-7.26 (m, 2H), 4.69- 4.34 (m, 2H), 4.34-4.24 (m, 1H), 3.40 (d, J = 8.0 Hz, 3H), 3.03 (d, J = 11.2 Hz, 3H), 2.86 (d, J = 4.8 Hz, 3H), 2.13-2.06 (m, 1H), 1.94-0.89 (m, 9H), 0.88-0.81 (m, 4H). |
| 195 | 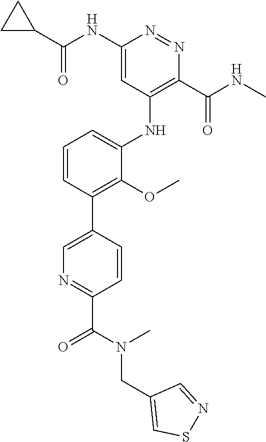 [SMILES: CNC(=O)c1nnc(NC(=O)C2CC2)cc1Nc1cccc(-c2ccc(C(=O)N(C)Cc3cnsc3)nc2)c1OC] | 573.1 [M + H]+ | |
| 196 | 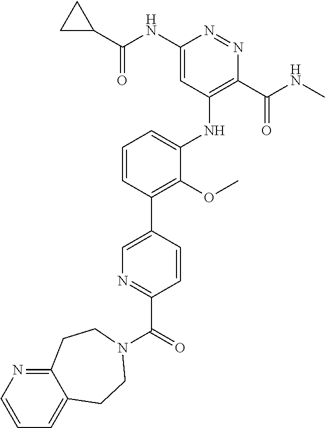 [SMILES: CNC(=O)c1nnc(NC(=O)C2CC2)cc1Nc1cccc(-c2ccc(C(=O)N3CCc4cccnc4CC3)nc2)c1OC] | 593.2 [M + H]+ | |
| 197 | 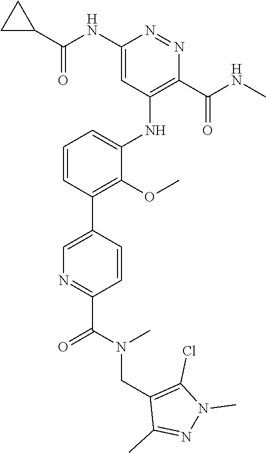 [SMILES: CNC(=O)c1nnc(NC(=O)C2CC2)cc1Nc1cccc(-c2ccc(C(=O)N(C)Cc3c(C)nn(C)c3Cl)nc2)c1OC] | 618.0 [M + H]+ | |
| 198 | 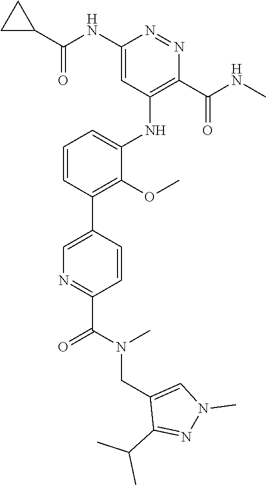 [SMILES: CNC(=O)c1nnc(NC(=O)C2CC2)cc1Nc1cccc(-c2ccc(C(=O)N(C)Cc3cn(C)nc3C(C)C)nc2)c1OC] | 612.2 [M + H]+ | |
| 199 | 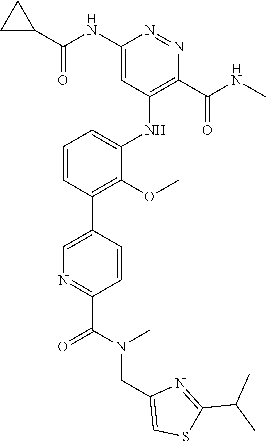 [SMILES: CNC(=O)c1nnc(NC(=O)C2CC2)cc1Nc1cccc(-c2ccc(C(=O)N(C)Cc3csc(C(C)C)n3)nc2)c1OC] | 615.2 [M + H]+ | |
| 207* | 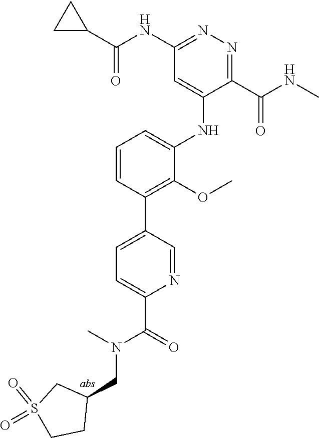 [SMILES: CNC(=O)c1nnc(NC(=O)C2CC2)cc1Nc1cccc(-c2ccc(C(=O)N(C)C[C@H]3CCS(=O)(=O)C3)nc2)c1OC] | 608.3 | ¹H NMR (DMSO-d6, 400 MHz) 11.36 (1H, s), 11.02 (1H, d, J = 8.6 Hz), 9.19 (1H, q, J = 4.7 Hz), 8.81-8.76 (1H, m), 8.21 (1H, d, J = 3.2 Hz), 8.14 (1H, ddd, J = 8.1, 3.6, 2.2 Hz), 7.71 (1H, dd, J = 8.1, 0.9 Hz), 7.55 (1H, dd, J = 7.5, 2.2 Hz), 7.40-7.29 (2H, m), 3.73 (1H, dd, J = 13.2, 7.0 Hz), 3.55 (1H, td, J = 12.0, 10.6, 6.7 Hz), 3.41 (3H, d, J = 2.1 Hz), 3.30- 3.20 (1H, m), 3.17-2.99 (4H, m), 2.98-2.76 (5H, m), 2.30 (1H, dt, J = 7.9, 4.4 Hz), 2.13 (2H, dp, J = 18.5, 6.5 Hz), 1.99-1.62 (1H, m), 0.89-0.82 (4H, m). |
| 209* | 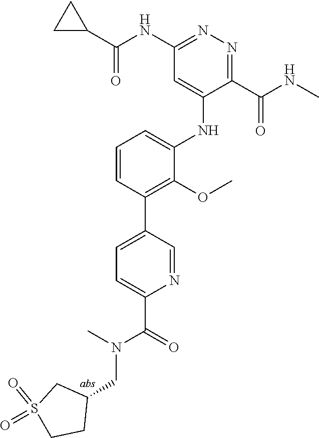 [SMILES: CNC(=O)c1nnc(NC(=O)C2CC2)cc1Nc1cccc(-c2ccc(C(=O)N(C)C[C@@H]3CCS(=O)(=O)C3)nc2)c1OC] | 608.3 | ¹H NMR (DMSO-d6, 400 MHz) 11.36 (1H, s), 11.02 (1H, d, J = 8.6 Hz), 9.19 (1H, q, J = 4.7 Hz), 8.81-8.76 (1H, m), 8.21 (1H, d, J = 3.2 Hz), 8.14 (1H, ddd, J = 8.1, 3.6, 2.2 Hz), 7.71 (1H, dd, J = 8.1, 0.9 Hz), 7.55 (1H, dd, J = 7.5, 2.2 Hz), 7.40-7.29 (2H, m), 3.73 (1H, dd, J = 13.2, 7.0 Hz), 3.55 (1H, td, J = 12.0, 10.6, 6.7 Hz), 3.41 (3H, d, J = 2.1 Hz), 3.30- 3.20 (1H, m), 3.17-2.99 (4H, m), 2.98-2.76 (5H, m), 2.30 (1H, dt, J = 7.9, 4.4 Hz), 2.13 (2H, dp, J = 18.5, 6.5 Hz), 1.99-1.62 (1H, m), 0.89-0.82 (4H, m). |
| 210 | 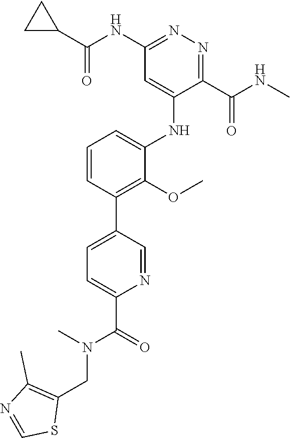 [SMILES: CNC(=O)c1nnc(NC(=O)C2CC2)cc1Nc1cccc(-c2ccc(C(=O)N(C)Cc3scnc3C)nc2)c1OC] | 587.2 [M + H]+ | |
| 211 | 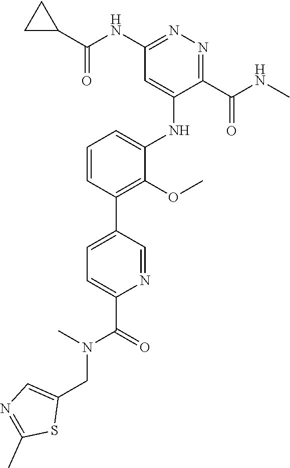 [SMILES: CNC(=O)c1nnc(NC(=O)C2CC2)cc1Nc1cccc(-c2ccc(C(=O)N(C)Cc3cnc(C)s3)nc2)c1OC] | 587.2 [M + H]+ | |
| 222 | 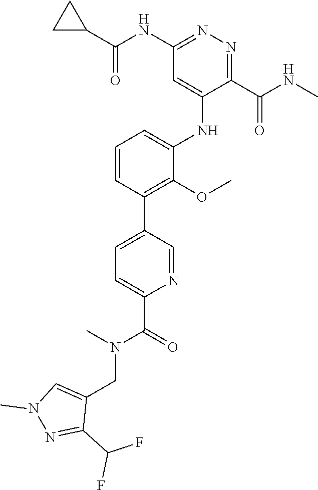 | 620.2 [M + H]+ | ¹H NMR (400 MHz, DMSO- d6) 11.37 (s, 1H), 11.01 (s, 1H), 9.20 (d, J = 5.0 Hz, 1H), 8.78 (d, J = 2.1 Hz, 1H), 8.20 (s, 1H), 7.85 (s, 1H), 7.71 (dd, J = 8.1, 2.0 Hz, 1H), 7.54 (m, J = 7.3, 2.3 Hz, 1H), 7.39-7.28 (m, 2H), 6.78- 6.94 (m, 1H), 4.62 (d, J = 8.5 Hz, 2H), 3.87 (d, J = 11.3 Hz, 3H), 3.42-3.36 (m, 4H), 2.97 (d, J = 1.6 Hz, 3H), 2.86 (d, J = 4.8 Hz, 3H), 2.10 (m, J = 10.0, 5.7 Hz, 1H), 0.88-0.81 (m, 4H). |
| 225* | 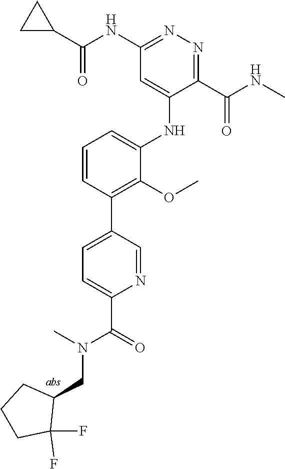 [SMILES: CNC(=O)c1nnc(NC(=O)C2CC2)cc1Nc1cccc(-c2ccc(C(=O)N(C)C[C@@H]3CCCC3(F)F)nc2)c1OC] | 594.2 [M + H]+ | ¹H NMR (DMSO-d6, 400 MHz) 11.36 (1H, s), 11.00 (1H, d, J = 2.9 Hz), 9.18 (1H, q, J = 4.9 Hz), 8.77 (1H, dd, J = 5.4, 2.1 Hz), 8.20 (1H, s), 8.12 (1H, dd, J = 8.1, 2.2 Hz), 7.66 (1H, dd, J = 8.1, 3.4 Hz), 7.54 (1H, dd, J = 7.4, 2.2 Hz), 7.39-7.28 (2H, m), 3.55 (2H, dd, J = 11.1, 7.6 Hz), 3.38-3.40 (2H, m), 3.02 (3H, d, J = 15.7 Hz), 2.86 (3H, d, J = 4.8 Hz), 2.59 (1H, td, J = 17.1, 16.5, 8.3 Hz), 2.37-2.15 (2H, m), 2.14- 2.03 (2H, m), 1.93 (2H, qd, J = 12.5, 10.6, 7.0 Hz), 1.85- 1.49 (2H, m), 0.88-0.81 (4H, m). |
| 226* | 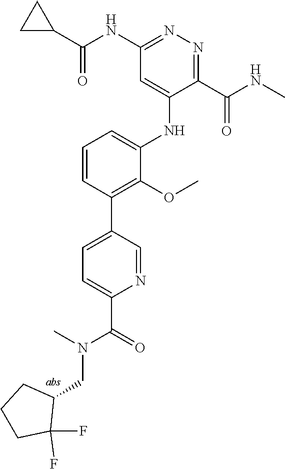 [SMILES: CNC(=O)c1nnc(NC(=O)C2CC2)cc1Nc1cccc(-c2ccc(C(=O)N(C)C[C@H]3CCCC3(F)F)nc2)c1OC] | 594.2 [M + H]+ | ¹H NMR (DMSO-d6, 400 MHz) 11.36 (1H, s), 11.00 (1H, d, J = 2.9 Hz), 9.18 (1H, q, J = 4.9 Hz), 8.77 (1H, dd, J = 5.4, 2.1 Hz), 8.20 (1H, s), 8.12 (1H, dd, J = 8.1, 2.2 Hz), 7.66 (1H, dd, J = 8.1, 3.4 Hz), 7.54 (1H, dd, J = 7.4, 2.2 Hz), 7.39-7.28 (2H, m), 3.55 (2H, dd, J = 11.1, 7.6 Hz), 3.38-3.40 (2H, m), 3.02 (3H, d, J = 15.7 Hz), 2.86 (3H, d, J = 4.8 Hz), 2.59 (1H, td, J = 17.1, 16.5, 8.3 Hz), 2.37-2.15 (2H, m), 2.14- 2.03 (2H, m), 1.93 (2H, qd, J = 12.5, 10.6, 7.0 Hz), 1.85- 1.49 (2H, m), 0.88-0.81 (4H, m). |
| 244 | 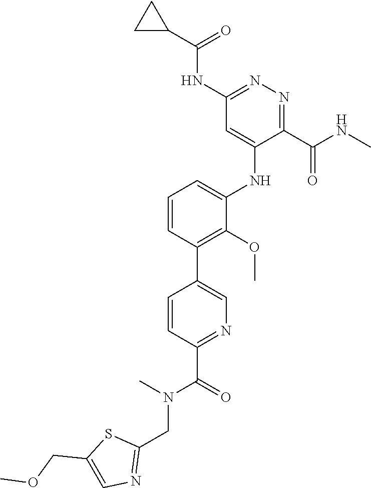 | 617.3 [M + H]+ | ¹H NMR (400 MHz, DMSO- d6) 11.36 (s, 1H), 11.01 (d, J = 3.2 Hz, 1H), 9.19 (d, J = 5.3 Hz, 1H), 8.84-8.76 (m, 1H), 8.23-8.13 (m, 2H), 7.82- 7.65 (m, 2H), 7.55 (dt, J = 6.0, 3.2 Hz, 1H), 7.40-7.31 (m, 2H), 4.99 (d, J = 27.0 Hz, 2H), 4.62 (d, J = 12.6 Hz, 2H), 3.40 (d, J = 9.9 Hz, 3H), 3.28 (d, J = 12.2 Hz, 3H), 3.10 (d, J = 26.8 Hz, 3H), 2.86 (d, J = 4.8 Hz, 3H), 2.10 (s, 1H), 0.84 (d, J = 5.7 Hz, 4H). |
| 252 | 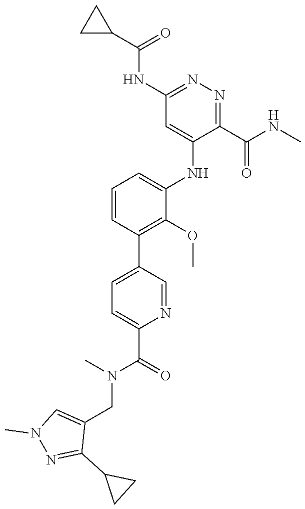 | 610.4 [M + H]+ | ¹H NMR (400 MHz, DMSO- d6) 11.36 (s, 1H), 11.00 (s, 1H), 9.19 (q, J = 4.7 Hz, 1H), 8.79 (m, 1H), 8.20 (s, 1H), 8.13 (m, 1H), 7.73-7.63 (m, 1H), 7.60-7.50 (m, 2H), 7.39- 7.24 (m, 2H), 4.57 (d, J = 27.4 Hz, 2H), 3.70 (d, J = 13.2 Hz, 3H), 3.40 (s, 3H), 2.94 (d, J = 16.7 Hz, 3H), 2.86 (d, J = 4.8 Hz, 3H), 2.15-2.04 (m, 1H), 1.97 (m, 1H), 0.88-0.77 (m, 5H), 0.77- 0.57 (m, 3H). |
| 253 | 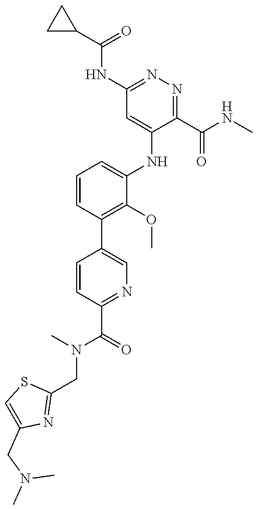 [SMILES: CNC(=O)c1nnc(NC(=O)C2CC2)cc1Nc1cccc(-c2ccc(C(=O)N(C)Cc3nc(CN(C)C)cs3)nc2)c1OC] | 630.3 [M + H]+ | ¹H NMR (Chloroform-d, 400 MHz) 11.15 (1H, d, J = 10.8 Hz), 9.09 (1H, s), 8.84 (1H, dd, J = 4.5, 2.1 Hz), 8.31 (1H, d, J = 1.6 Hz), 8.21-8.09 (2H, m), 7.90 (1H, dd, J = 49.9, 8.1 Hz), 7.54 (1H, dd, J = 7.9, 1.5 Hz,), 7.32 (1H, td, J = 8.8, 8.3, 2.5 Hz), 7.27-7.18 (2H, m), 5.11 (2H, d, J = 28.7 Hz), 3.68 (2H, d, J = 9.2 Hz), 3.47 (3H, d, J = 4.4 Hz), 3.24 (3H, d, J = 33.4 Hz), 3.07 (3H, d, J = 5.0 Hz), 2.38 (6H, d, J = 6.9 Hz), 1.76 (1H, tt, J = 8.0, 4.4 Hz), 1.14 (2H, q, J = 4.1 Hz), 0.96 (2H, dq, J = 7.7, 4.2 Hz). |
| 257 | 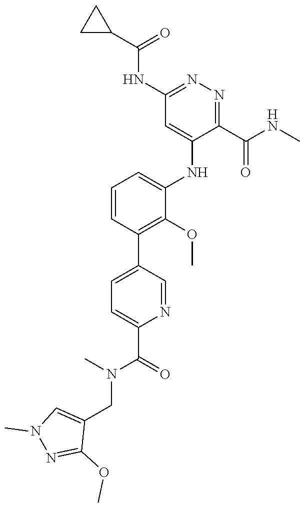 | 600.4 [M + H]+ | ¹H NMR (400 MHz, DMSO- d6) 11.36 (s, 1H), 11.01 (d, J = 3.0 Hz, 1H), 9.19 (q, J = 4.7 Hz, 1H), 8.81 (m, 1H), 8.20 (d, J = 2.1 Hz, 1H), 8.13 (m, 1H), 7.66 (m, 1H), 7.59- 7.47 (m, 2H), 7.40-7.28 (m, 2H), 4.37 (s, 1H), 4.31 (s, 1H), 3.82 (s, 2H), 3.72-3.62 (m, 4H), 3.40 (d, J = 5.4 Hz, 3H), 2.93 (d, J = 1.4 Hz, 3H), 2.86 (d, J = 4.8 Hz, 3H), 2.15-2.04 (m, 1H), 0.88-0.81 (m, 4H). |
| 261 | 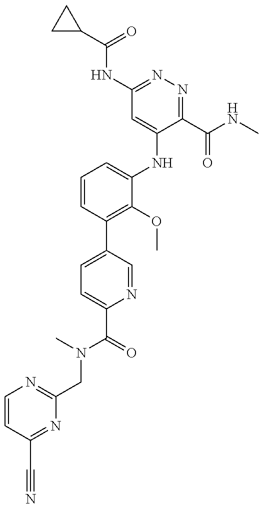 [SMILES: CNC(=O)c1nnc(NC(=O)C2CC2)cc1Nc1cccc(-c2ccc(C(=O)N(C)Cc3nccc(C#N)n3)nc2)c1OC] | 593.3 [M + H]+ | ¹H NMR (400 MHz, Chloroform-d) 11.21 (d, J = 4.8 Hz, 1H), 9.24 (s, 1H), 8.99 (dd, J = 15.4, 4.9 Hz, 1H), 8.74 (ddd, J = 108.8, 2.2, 0.9 Hz, 1H), 8.31 (d, J = 10.8 Hz, 1H), 8.18-8.02 (m, 2H), 7.89 (ddd, J = 28.0, 8.1, 0.8 Hz, 1H), 7.60-7.48 (m, 2H), 7.32 (dt, J = 17.0, 7.9 Hz, 1H), 7.20 (ddd, J = 30.9, 7.7, 1.6 Hz, 1H), 5.22 (s, 1H), 5.08 (s, 1H), 3.49 (s, 2H), 3.42 (s, 1H), 3.40 (s, 2H), 3.30 (s, 1H), 3.07 (dd, J = 5.1, 3.8 Hz, 3H), 1.76 (dq, J = 8.0, 3.6 Hz, 1H), 1.20-1.07 (m, 2H), 0.97 (tdd, J = 7.6, 4.1, 2.0 Hz, 2H). |
| 271 | 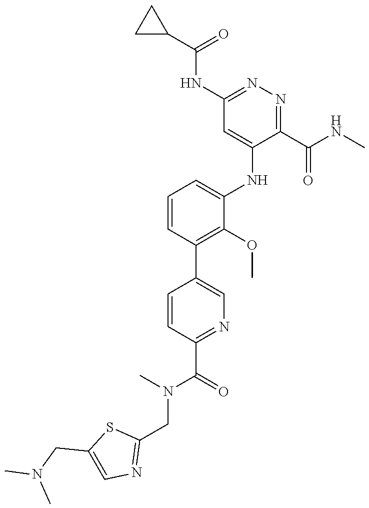 | 630.4 [M + H]+ | ¹H NMR (400 MHz, DMSO- d6) 11.36 (s, 1H), 11.01 (s, 1H), 9.19 (s, 1H), 8.79 (d, J = 16.3 Hz, 1H), 8.23-8.11 (m, 2H), 7.76 (dd, J = 12.8, 8.0 Hz, 1H), 7.57 (d, J = 17.2 Hz, 2H), 7.34 (s, 2H), 4.97 (d, J = 26.1 Hz, 2H), 3.61 (d, J = 15.2 Hz, 2H), 3.40 (d, J = 10.1 Hz, 3H), 3.09 (d, J = 23.1 Hz, 3H), 2.86 (d, J = 4.8 Hz, 3H), 2.18 (s, 3H), 2.14 (s, 3H), 1.24 (s, 1H), 0.84 (d, J = 7.1 Hz, 4H). |
| 282 | 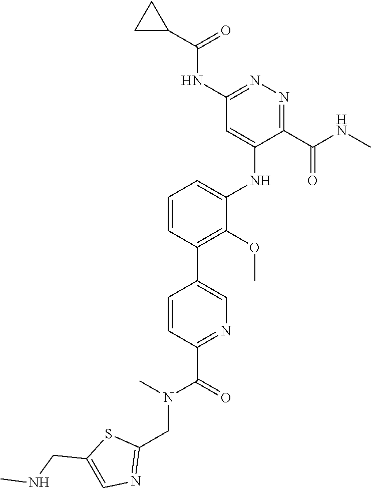 | 616.3 [M + H]+ | ¹H NMR (DMSO-d6, 400 MHz) 11.36 (1H, s), 11.01 (1H, d, J = 2.3 Hz), 9.19 (1H, d, J = 5.1 Hz), 8.84-8.76 (1H, m), 8.22-8.12 (2H, m), 7.76 (1H, dd, J = 18.7, 8.1 Hz), 7.60-7.51 (2H, m), 7.34 (2H, dd, J = 5.9, 3.4 Hz), 4.96 (2H, d, J = 27.3 Hz), 3.81 (2H, d, J = 13.8 Hz), 3.40 (3H, d, J = 8.3 Hz), 3.10 (1H, m), 3.08 (3H, d, J = 24.8 Hz), 2.86 (3H, d, J = 4.8 Hz), 2.26 (3H, d, J = 13.7 Hz), 2.14-2.06 (1H, m), 0.84 (4H, d, J = 6.6 Hz). |
| 284 | 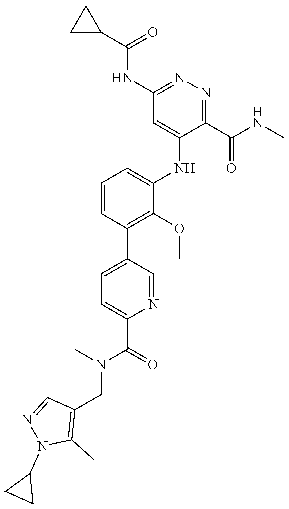 [SMILES: CNC(=O)c1nnc(NC(=O)C2CC2)cc1Nc1cccc(-c2ccc(C(=O)N(C)Cc3cnn(C4CC4)c3C)nc2)c1OC] | 610.4 [M + H]+ | ¹H NMR(400 MHz, DMSO- d6) 11.35 (s, 1H), 11.00 (s, 1H), 9.18 (q, J = 4.8 Hz, 1H), 8.75 (dd, J = 2.2, 0.9 Hz, 1H), 8.20 (s, 1H), 8.13 (m, 1H), 7.68 (m, 1H), 7.54 (m, 1H), 7.39-7.24 (m, 3H), 4.50 (s, 1H), 4.44 (s, 1H), 3.54- 3.37 (m, 4H), 2.91 (d, J = 17.6 Hz, 3H), 2.86 (d, J = 4.8 Hz, 3H), 2.39 (s, 2H), 2.16- 2.05 (m, 2H), 1.08-1.00 (m, 1H), 1.03-0.89 (m, 3H), 0.89- 0.81 (m, 4H). |
| 285 | 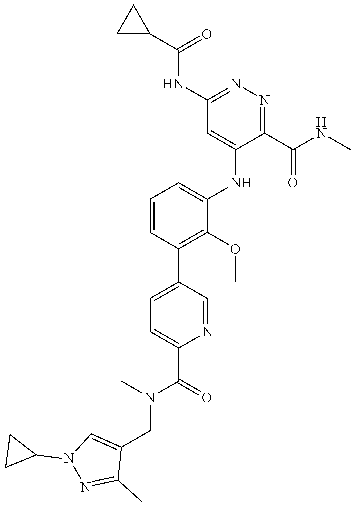 [SMILES: CNC(=O)c1nnc(NC(=O)C2CC2)cc1Nc1cccc(-c2ccc(C(=O)N(C)Cc3cn(C4CC4)nc3C)nc2)c1OC] | 610.4 [M + H]+ | ¹H NMR (400 MHz, DMSO- d6) 11.35 (s, 1H), 11.00 (s, 1H), 9.18 (q, J = 4.7 Hz, 1H), 8.83-8.73 (m, 1H), 8.19-8.13 (m, 2H), 7.73-7.58 (m, 2H), 7.57-7.50 (m, 1H), 7.43-7.28 (m, 2H), 4.50-4.44 (m, 2H), 3.59 (m, 1H), 3.40 (s, 3H), 2.94-2.84 (m, 6H), 2.17 (s, 2H), 2.15-2.05 (m, 1H), 1.92 (s, 1H), 0.96-0.81 (m, 8H) |
| 297 | 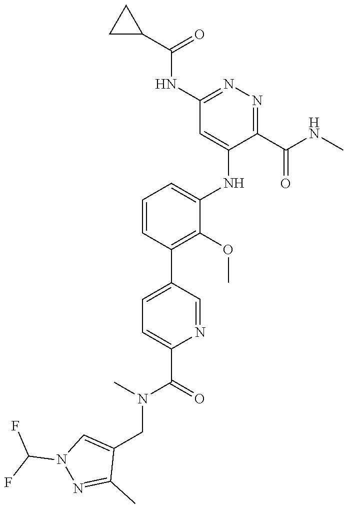 [SMILES: CNC(=O)c1nnc(NC(=O)C2CC2)cc1Nc1cccc(-c2ccc(C(=O)N(C)Cc3cn(C(F)F)nc3C)nc2)c1OC] | 620.4 [M + H]+ | ¹H NMR (400 MHz, DMSO- d6) 11.36 (s, 1H), 11.01 (s, 1H), 9.19 (q, J = 4.7 Hz, 1H), 8.83-8.75 (m, 1H), 8.20 (d, J = 1.3 Hz, 1H), 8.18-8.10 (m, 2H), 7.76-7.64 (m, 2H), 7.58- 7.49 (m, 1H), 7.40-7.28 (m, 2H), 4.57 (d, J = 16.7 Hz, 2H), 3.40 (d, J = 3.9 Hz, 3H), 2.95 (d, J = 14.7 Hz, 3H), 2.86 (d, J = 4.8 Hz, 3H), 2.27 (s, 2H), 2.15-2.07 (m, 1H), 2.05 (s, 1H), 0.90-0.81 (m, 4H). |
| 298 | 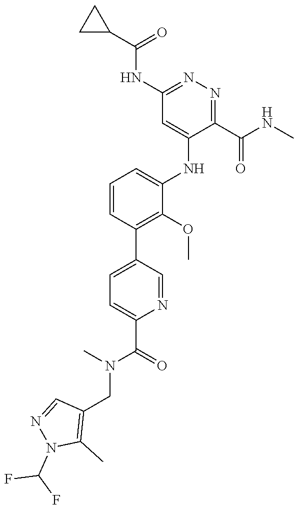 [SMILES: CNC(=O)c1nnc(NC(=O)C2CC2)cc1Nc1cccc(-c2ccc(C(=O)N(C)Cc3cnn(C(F)F)c3C)nc2)c1OC] | 620.3 [M + H]+ | ¹H NMR (400 MHz, DMSO- d6) 11.36 (s, 1H), 11.00 (s, 1H), 9.19 (q, J = 4.8 Hz, 1H), 8.76 (dd, J = 2.2, 0.9 Hz, 1H), 8.20 (s, 1H), 8.14 (td, J = 8.5, 2.2 Hz, 1H), 7.97-7.63 (m, 3H), 7.57-7.51 (m, 1H), 7.33 (m, 2H), 4.53 (d, J = 23.3 Hz, 2H), 3.40 (d, J = 1.5 Hz, 3H), 2.95 (d, J = 4.7 Hz, 3H), 2.86 (d, J = 4.8 Hz, 3H), 2.48 (s, 2H), 2.24 (s, 1H), 2.15-2.04 (m, 1H), 0.88-0.81 (m, 4H). |
| 332 | 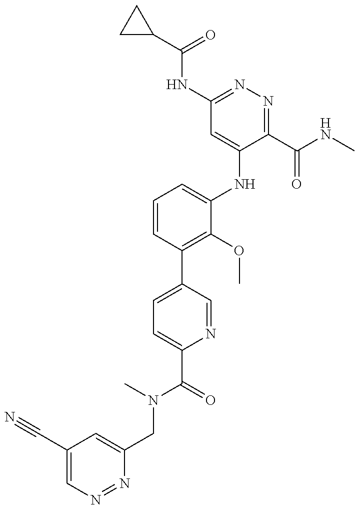 [SMILES: CNC(=O)c1nnc(NC(=O)C2CC2)cc1Nc1cccc(-c2ccc(C(=O)N(C)Cc3cc(C#N)cnn3)nc2)c1OC] | 593.3 [M + H]+ | ¹H NMR (DMSO-d6, 400 MHz) 11.35 (1H, d, J = 5.2 Hz), 10.99 (1H, d, J = 9.9 Hz), 9.60 (1H, dd, J = 14.3, 1.9 Hz), 91.9 (1H, q, J = 4.8 Hz), 8.73 (1H, dd, J = 73.4, 2.2 Hz), 8.29 (1H, dd, J = 24.5, 2.0 Hz), 8.22-8.08 (2H, m), 7.80 (1H, dd, J = 11.3, 8.1 Hz), 7.53 (1H, ddd, J = 11.5, 7.3, 2.3 Hz), 7.40-7.25 (2H, m), 5.09 (2H, d, J = 13.1 Hz), 3.41 (3H, s), 3.14 (3H, d, J = 37.1 Hz), 2.86 (3H, dd, J = 4.9, 2.6 Hz), 2.09 (1H, dt, J = 7.9, 4.6 Hz), 0.83 (4H, ddt, J = 7.0, 5.0, 2.4 Hz) |
| 362 | 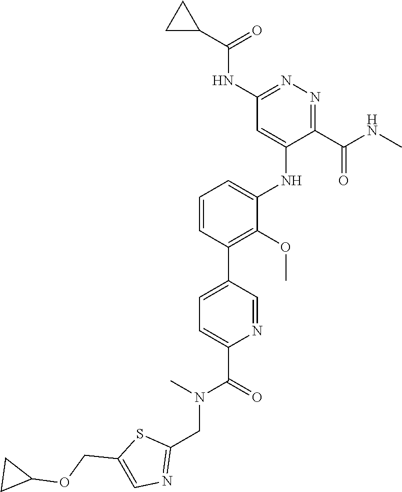 [SMILES: CNC(=O)c1nnc(NC(=O)C2CC2)cc1Nc1cccc(-c2ccc(C(=O)N(C)Cc3ncc(COC4CC4)s3)nc2)c1OC] | 643.3 [M + H]+ | ¹H NMR (400 MHz, DMSO- d6) 11.36 (s, 1H), 11.00 (d, J = 2.6 Hz, 1H), 9.19 (d, J = 5.2 Hz, 1H), 8.84-8.75 (m, 1H), 8.22-8.12 (m, 2H), 7.83- 7.72 (m, 1H, 7.69 (d, J = 14.6 Hz, 1H), 7.58-7.50 (m, 1H), 7.40-7.30 (m, 2H), 5.03 (s, 1H), 4.95 (s, 1H), 4.73- 4.66 (m, 2H), 3.40 (d, J = 10.1 Hz, 4H), 3.13 (s, 2H), 3.06 (s, 1H), 2.86 (d, J = 4.8 Hz, 3H), 2.14-2.04 (m, 1H), 0.84 (q, J = 3.4, 3.0 Hz, 4H), 0.59-0.40 (m, 4H). |
| 380 | 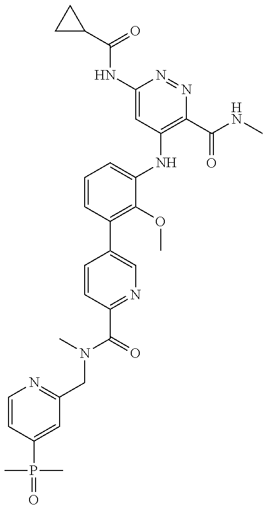 | 643.1 [M + H]+ | |
| 394 | 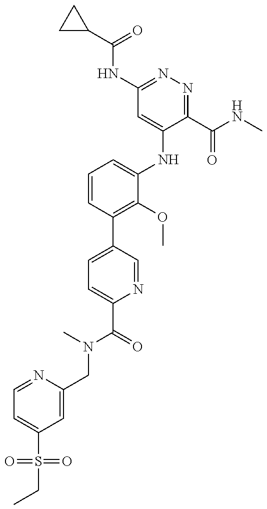 | 359.3 [M + H]+ | ¹H NMR (DMSO-d6, 400 MHz) 11.36 (1H, d, J = 5.5 Hz), 11.00 (1H, d, J = 13.7 Hz), 9.19 (1H, p, J = 4.7 Hz), 8.90 (1H, dd, J = 25.6, 5.0 Hz), 8.73 (1H, dd, J = 69.8, 2.2 Hz), 8.23-8.05 (2H, m), 7.87-7.69 (3H, m), 7.54 (1H, ddd, J = 12.4, 7.2, 2.3 Hz), 7.40- 7.24 (2H, m), 4.97 (2H, d, J = 9.7 Hz), 3.46 (2H, q, J = 7.5 Hz), 3.37 (3H, d, J = 19.9 Hz), 3.11 (3H, d, J = 33.5 Hz), 2.86 (3H, dd, J = 4.8, 3.1 Hz), 2.09 (1H, qd, J = 6.8, 4.4 Hz), 1.12 (3H, dt, J = 22.5, 7.4 Hz), 0.89-0.77 (4H, m). |
| 405* | 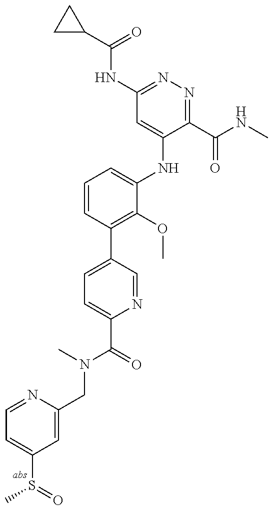 [SMILES: CNC(=O)c1nnc(NC(=O)C2CC2)cc1Nc1cccc(-c2ccc(C(=O)N(C)Cc3cc([S@@](C)=O)ccn3)nc2)c1OC] | 629.3 [M + H]+ | ¹H NMR (400 MHz, DMSO- d6) 11.34 (d, J = 5.1 Hz, 1H), 10.99 (d, J = 12.6 Hz, 1H), 9.17 (p, J = 4.7 Hz, 1H), 8.79 (dd, J = 23.7, 3.6 Hz, 1H), 8.68 (t, J = 3.9 Hz, 1H), 8.23- 8.03 (m, 2H), 7.77-7.62 (m, 3H), 7.59-7.50 (m, 1H), 7.39- 7.24 (m, 2H), 4.91 (d, J = 7.5 Hz, 2H), 3.38 (d, J = 26.4 Hz, 3H), 3.10 (d, J = 29.5 Hz, 3H), 2.86 (q, J = 2.9 Hz, 6H), 2.09 (qd, J = 7.4, 6.8, 4.2 Hz, 1H), 0.84 (qd, J = 7.4, 5.4, 2.4 Hz, 4H). |
| 407* | 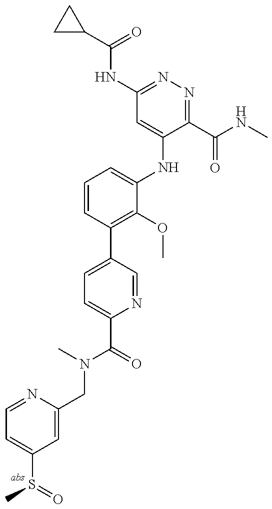 [SMILES: CNC(=O)c1nnc(NC(=O)C2CC2)cc1Nc1cccc(-c2ccc(C(=O)N(C)Cc3cc([S@](C)=O)ccn3)nc2)c1OC] | 629.3 [M + H]+ | ¹H NMR (400 MHz, DMSO- d6) 11.34 (d, J = 5.1 Hz, 1H), 10.99 (d, J = 12.6 Hz, 1H), 9.17 (p, J = 4.7 Hz, 1H), 8.79 (dd, J = 23.7, 3.6 Hz, 1H), 8.68 (t, J = 3.9 Hz, 1H), 8.23- 8.03 (m, 2H), 7.77-7.62 (m, 3H), 7.59-7.50 (m, 1H), 7.39- 7.24 (m, 2H), 4.91 (d, J = 7.5 Hz, 2H), 3.38 (d, J = 26.4 Hz, 3H), 3.10 (d, J = 29.5 Hz, 3H), 2.86 (q, J = 2.9 Hz, 6H), 2.09 (qd, J = 7.4, 6.8, 4.2 Hz, 1H), 0.84 (qd, J = 7.4, 5.4, 2.4 Hz, 4H). |

*Absolute stereochemistry arbitrarily assigned.

### Example 20

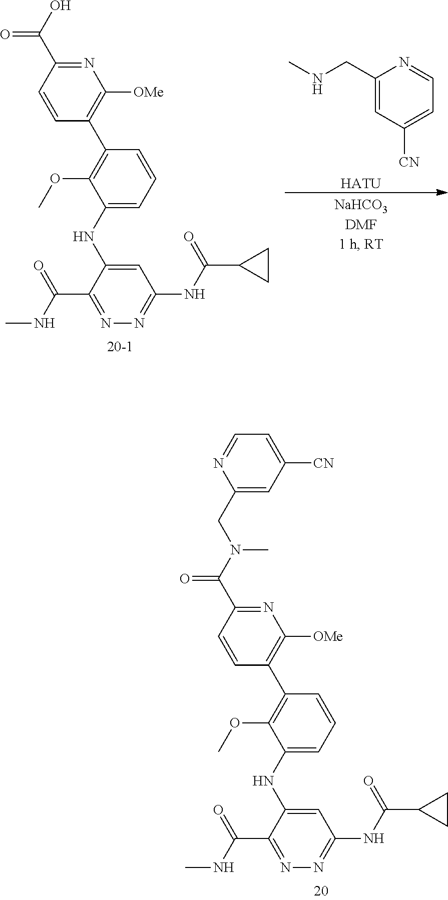 [SMILES: CNC(=O)c1nnc(NC(=O)C2CC2)cc1Nc1cccc(-c2ccc(C(=O)O)nc2OC)c1OC ; CNCc1cc(C#N)ccn1 ; "HATU" ; CNC(=O)c1nnc(NC(=O)C2CC2)cc1Nc1cccc(-c2ccc(C(=O)N(C)Cc3cc(C#N)ccn3)nc2OC)c1OC]

Compound 20-1 was prepared using analogous procedures as compound 7-2 in Example 7 except that methyl 5-bromo-6-methoxypicolinate was used in place of methyl 5-bromopyrazine-2-carboxylate.

Compound 20 was then prepared using analogous coupling conditions as compound 18 in example 18.

LC/MS (ES+): m/z=622.4 [M+H]+

¹H NMR (500 MHz, DMSO) δ 11.32 (d, J=7.0 Hz, 1H), 10.91 (d, J=18.0 Hz, 1H), 9.15 (d, J=5.4 Hz, 1H), 8.82 (dd, J=22.6, 5.0 Hz, 1H), 8.17 (d, J=17.2 Hz, 1H), 7.91-7.79 (m, 1H), 7.76 (t, J=6.8 Hz, 1H), 7.51-7.41 (m, 1H), 7.37 (dd, J=26.6, 7.5 Hz, 1H), 7.24 (dt, J=22.4, 7.9 Hz, 1H), 7.17-7.01 (m, 1H), 4.89 (d, J=26.9 Hz, 2H), 3.40 (d, J=2.9 Hz, 3H), 3.33 (s, 3H), 3.13 (d, J=66.2 Hz, 3H), 2.84 (t, J=4.7 Hz, 3H), 2.09 (d, J=4.6 Hz, 1H), 0.83 (d, J=5.6 Hz, 4H).

### Example 21

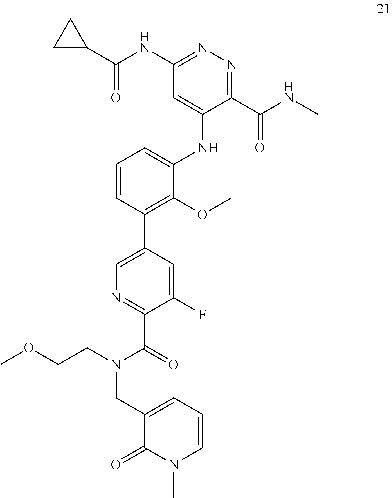

Compound 21 was prepared in analogous fashion to compound 14-2 in Example 14 except that compound 28-1 was used in place of compound 8-2.

LC/MS m/z (ES+): 659.7 [M+H]+

¹H NMR (500 MHz, DMSO): δ 11.36 (d, J=4.1 Hz, 1H), 10.98 (d, J=7.6 Hz, 1H), 9.19-9.12 (m, 1H), 8.66 (t, J=1.6 Hz, 1H), 8.13-8.05 (m, 2H), 7.67 (dd, J=6.7, 2.0 Hz, 1H), 7.54 (ddd, J=9.3, 7.1, 2.4 Hz, 1H), 7.44-7.28 (m, 3H), 6.28 (t, J=6.8 Hz, 1H), 4.50 (s, 1H), 4.34 (s, 1H), 3.68 (t, J=5.7 Hz, 1H), 3.56 (t, J=5.6 Hz, 1H), 3.49 (d, J=5.4 Hz, 1H), 3.48 (s, 2H), 3.43-3.33 (m, 6H), 3.28 (s, 1H), 3.08 (s, 2H), 2.85 (dd, J=4.8, 1.4 Hz, 3H), 2.12-2.02 (m, 1H), 0.88-0.78 (m, 4H)

### Example 22

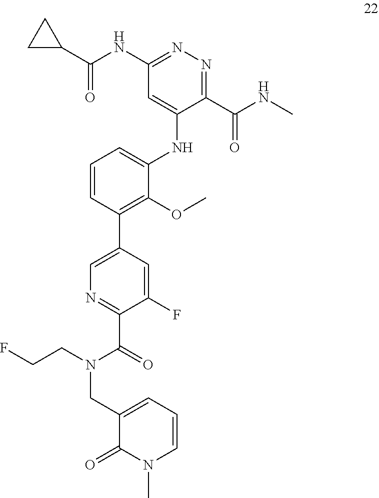 [SMILES: CNC(=O)c1nnc(NC(=O)C2CC2)cc1Nc1cccc(-c2cnc(C(=O)N(CCF)Cc3cccn(C)c3=O)c(F)c2)c1OC]

Compound 22 was prepared in analogous fashion to compound 14-2 in Example 14 except that 2,2-difluoroethan-1-amine was used in place of 1-amino-2-methoxyethane and compound 28-1 was used in place of compound 8-2.

LC/MS m/z (ES+): 647.5 [M+H]+

¹H NMR (500 MHz, DMSO): δ 11.35 (d, J=3.2 Hz, 1H), 10.97 (d, J=6.6 Hz, 1H), 9.16 (qd, J=4.8, 2.1 Hz, 1H), 8.66 (d, J=1.6 Hz, 1H), 8.59 (d, J=1.6 Hz, OH), 8.13 (d, J=6.5 Hz, 1H), 8.09 (dd, J=10.5, 1.7 Hz, 1H), 8.00 (dd, J=10.6, 1.7 Hz, OH), 7.68 (dd, J=6.7, 2.0 Hz, 1H), 7.61 (dd, J=6.7, 2.0 Hz, OH), 7.54 (td, J=7.4, 2.6 Hz, 1H), 7.40-7.29 (m, 3H), 7.08 (dd, J=6.9, 1.9 Hz, OH), 6.29 (t, J=6.8 Hz, 1H), 6.16 (t, J=6.8 Hz, OH), 4.71 (t, J=5.0 Hz, OH), 4.62 (t, J=5.0 Hz, OH), 4.55 (t, J=4.7 Hz, 1H), 4.53 (s, 1H), 4.46 (t, J=4.7 Hz, 1H), 4.37 (s, 1H), 3.89 (t, J=5.0 Hz, OH), 3.84 (t, J=5.0 Hz, OH), 3.69 (dt, J=26.7, 4.8 Hz, 1H), 3.48 (s, 2H), 3.42 (s, 2H), 3.37 (d, J=7.8 Hz, 3H), 2.85 (d, J=4.8 Hz, 3H), 2.08 (ttd, J=7.5, 5.1, 2.4 Hz, 1H), 1.09 (s, 1H), 0.83 (dt, J=8.4, 3.3 Hz, 4H).

### Example 24

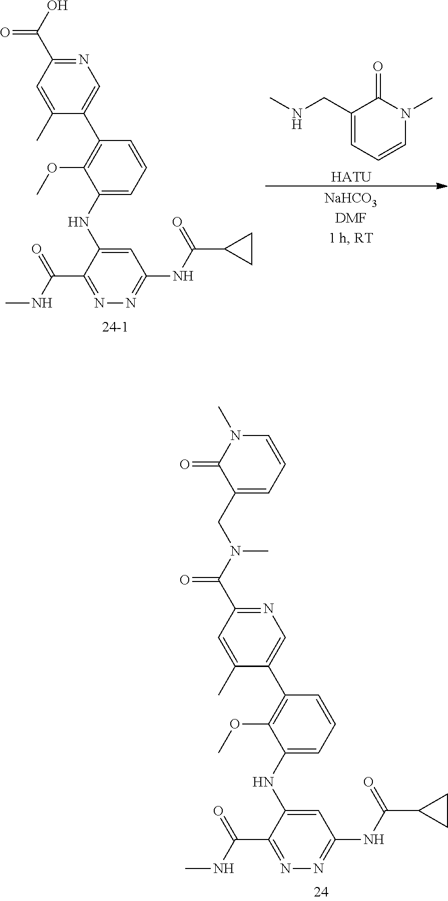

Compound 24-1 was prepared using analogous procedures as compound 7-2 in Example 7 except that methyl 5-bromo-4-methylpicolinate was used in place of methyl 5-bromopyrazine-2-carboxylate.

Compound 24 was then prepared using analogous coupling conditions as compound 18 in example 18.

¹H NMR (500 MHZ, DMSO) δ 11.32 (d, J=2.8 Hz, 1H), 10.99 (d, J=6.1 Hz, 1H), 9.15 (dt, J=6.0, 2.9 Hz, 1H), 8.14 (d, J=5.6 Hz, 1H), 7.80 (d, J=16.1 Hz, 1H), 7.66 (ddd, J=8.5, 6.7, 2.0 Hz, 1H), 7.51 (tdd, J=9.6, 5.4, 1.7 Hz, 2H), 7.34-7.25 (m, 2H), 6.25 (dt, J=21.5, 6.8 Hz, 1H), 4.50 (s, 1H), 4.18 (s, 1H), 3.50-3.44 (m, 5H), 2.98 (s, 2H), 2.88-2.81 (m, 5H), 2.47-2.36 (m, 1H), 2.32 (d, J=1.8 Hz, 3H), 2.30 (s, 2H), 2.08 (pt, J=5.2, 2.5 Hz, 1H), 0.82 (dq, J=6.8, 4.3, 3.6 Hz, 4H).

### Example 25

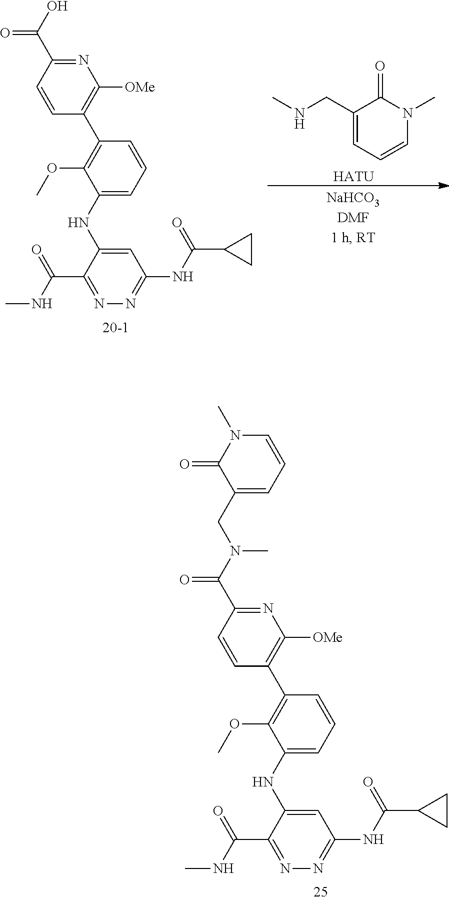

Compound 25 was prepared using analogous coupling conditions as compound 18 in example 18.

¹H NMR (500 MHz, DMSO) δ 11.33 (d, J=5.4 Hz, 1H), 10.92 (d, J=13.8 Hz, 1H), 9.14 (p, J=4.7 Hz, 1H), 8.14 (d, J=12.7 Hz, 1H), 7.75 (d, J=7.5 Hz, 1H), 7.64 (ddd, J=15.8, 6.8, 2.0 Hz, 1H), 7.46 (ddd, J=14.2, 8.0, 1.6 Hz, 1H), 7.32 (dd, J=12.7, 7.3 Hz, 2H), 7.23 (dt, J=18.2, 7.8 Hz, 1H), 7.12 (ddd, J=22.9, 7.7, 1.6 Hz, 1H), 6.26 (q, J=6.7 Hz, 1H), 4.46 (d, J=2.6 Hz, 2H), 3.87 (s, 1H), 3.47 (s, 1H), 3.39 (d, J=9.8 Hz, 3H), 3.32 (s, 2H), 3.16 (s, 1H), 3.10 (s, 1H), 2.99 (s, 2H), 2.84 (dd, J=4.8, 3.2 Hz, 3H), 2.07 (tt, J=9.8, 3.3 Hz, 1H), 0.82 (dq, J=7.3, 3.5 Hz, 4H).

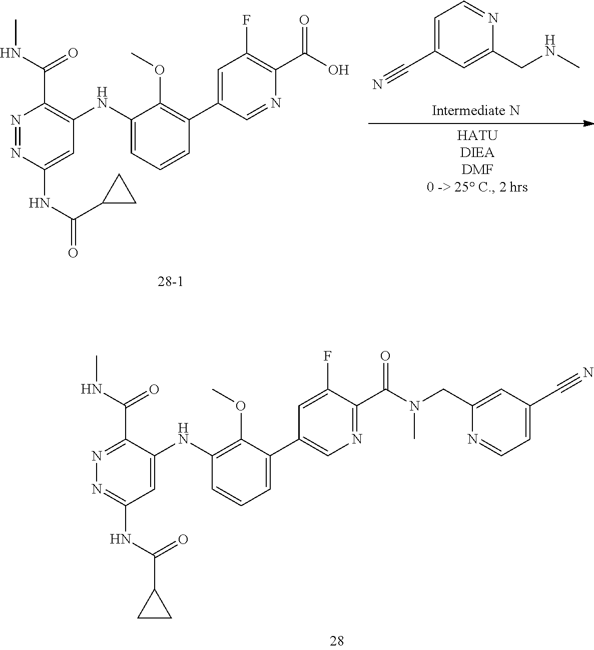

Compound 28-1 was prepared in analogous fashion to Intermediate M except that tert-butyl 5-bromo-3-fluoropicolinate was used in place of tert-butyl 5-bromo-picolinate. Compound 28-1 was then converted to desired amide 28 using the coupling conditions in Example 18.

LCMS m/z (ES+): 610.5 (M+H)+.

TABLE 4
The following compounds were prepared under analogous conditions as
compound 28 in example 28:

| | | LCMS m/z | |
|---|---|---|---|
| Compound | Structure | (ES+) | ¹H NMR |
| 27 | 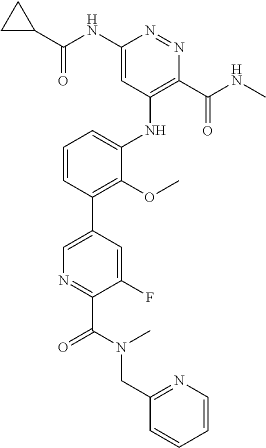 [SMILES: CNC(=O)c1nnc(NC(=O)C2CC2)cc1Nc1cccc(-c2cnc(C(=O)N(C)Cc3ccccn3)c(F)c2)c1OC] | 585.3 [M + H]+ | |
| 29 | 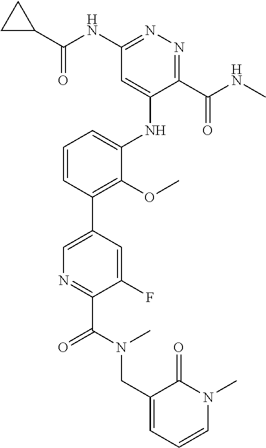 [SMILES: CNC(=O)c1nnc(NC(=O)C2CC2)cc1Nc1cccc(-c2cnc(C(=O)N(C)Cc3cccn(C)c3=O)c(F)c2)c1OC] | 615.3 [M + H]+ | |
| 48 | 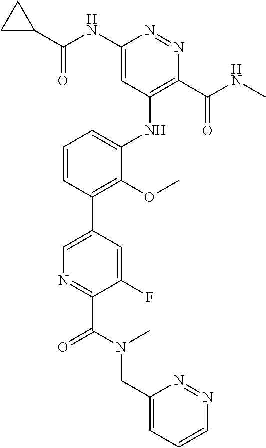 [SMILES: CNC(=O)c1nnc(NC(=O)C2CC2)cc1Nc1cccc(-c2cnc(C(=O)N(C)Cc3cccnn3)c(F)c2)c1OC] | 586.4 [M + H]+ | |
| 53 | 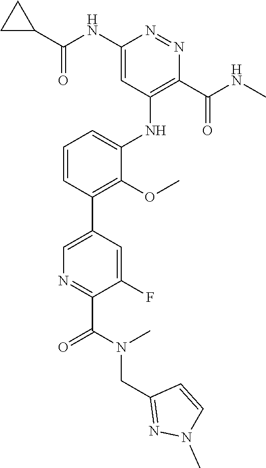 [SMILES: CNC(=O)c1nnc(NC(=O)C2CC2)cc1Nc1cccc(-c2cnc(C(=O)N(C)Cc3ccn(C)n3)c(F)c2)c1OC] | 588.4 [M + H]+ | |
| 98 | 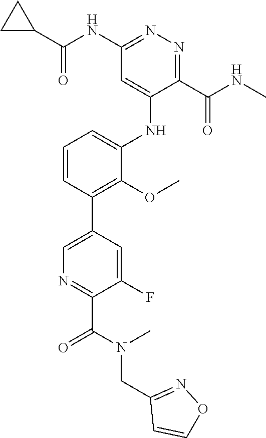 [SMILES: CNC(=O)c1nnc(NC(=O)C2CC2)cc1Nc1cccc(-c2cnc(C(=O)N(C)Cc3ccon3)c(F)c2)c1OC] | 575.5 [M + H]+ | ¹H NMR (400 MHz, DMSO-d6) 11.36 (s, 1H), 10.99 (d, J = 2.7 Hz, 1H), 9.18 (q, J = 4.8 Hz, 1H), 8.98-8.88 (m, 1H), 8.73- 8.65 (m, 1H), 8.18 (d, J = 3.0 Hz, 1H), 8.16-8.05 (m, 1H), 7.60- 7.52 (m, 1H), 7.41-7.30 (m, 2H), 6.60-6.52 (m, 1H), 4.72 (d, J = 95.9 Hz, 2H), 3.43 (d, J = 5.6 Hz, 3H), 3.00 (d, J = 51.6 Hz, 3H), 2.87 (d, J = 4.8 Hz, 3H), 2.11 (q, J = 6.2 Hz, 1H), 0.90- 0.81 (m, 4H). |
| 172 | 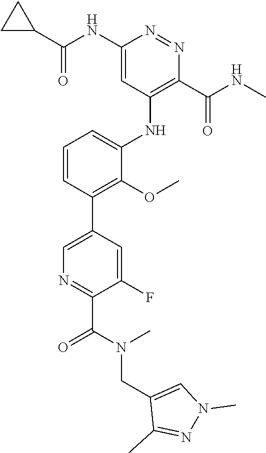 [SMILES: CNC(=O)c1nnc(NC(=O)C2CC2)cc1Nc1cccc(-c2cnc(C(=O)N(C)Cc3cn(C)nc3C)c(F)c2)c1OC] | 602.6 [M + H]+ | ¹H NMR (400 MHz, DMSO) δ 11.35 (s, 1H), 10.98 (s, 1H), 9.18 (d, J = 4.9 Hz, 1H), 8.68 (dt, J = 15.0, 1.7 Hz, 1H), 8.16 (s, 1H), 8.09 (dt, J = 10.5, 1.9 Hz, 1H), 7.65-7.45 (m, 2H), 7.36 (td, J = 4.3, 3.7, 2.3 Hz, 2H), 4.38 (d, J = 116.3 Hz, 2H), 3.73 (d, J = 15.0 Hz, 3H), 3.42 (s, 3H), 3.05- 2.73 (m, 6H), 2.20-1.86 (m, 4H), 0.83 (dd, J = 6.4, 3.5 Hz, 4H). |

### Example 32

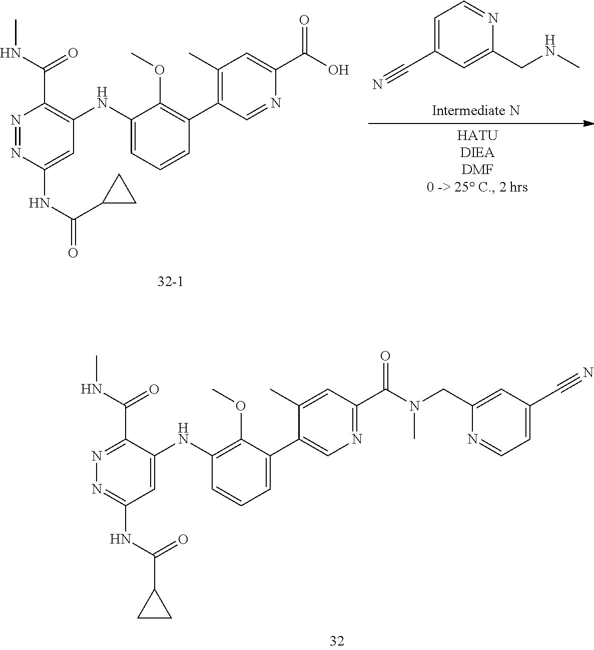

Compound 32-1 was prepared in analogous fashion to Intermediate M except that tert-butyl 5-bromo-4-methylpicolinate was used in place of tert-butyl 5-bromo-picolinate. Compound 32-1 was then converted to desired amide 32 using the coupling conditions in Example 18.

LCMS (ES+) m/z=593.4 [M+H]+

### Example 33

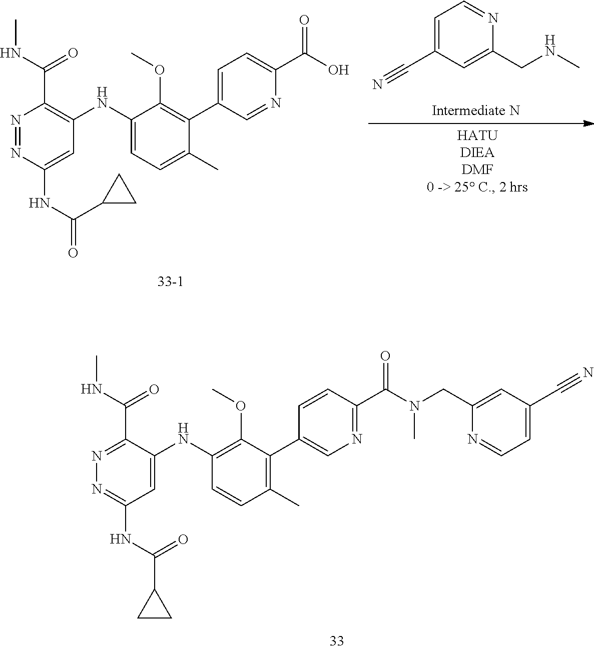

Compound 33-1 was prepared in analogous fashion to Intermediate M except that Intermediate I was used in place of Intermediate H. Compound 33-1 was then converted to desired amide 33 using the coupling conditions in Example 18.

LCMS m/z (ES+): 606.3 (M+H)+

¹H NMR (400 MHZ, DMSO-d6) 11.32 (d, J=5.8 Hz, 1H), 10.81 (d, J=13.5 Hz, 1H), 9.15 (t, J=5.0 Hz, 1H), 8.81 (dd, J=30.3, 5.0 Hz, 1H), 8.51 (dd, J=62.4, 2.1 Hz, 1H), 8.13 (d, J=13.5 Hz, 1H), 8.01-7.63 (m, 4H), 7.42 (dd, J=12.5, 8.2 Hz, 1H), 7.20 (dd, J=13.2, 8.3 Hz, 1H), 4.90 (d, J=11.7 Hz, 2H), 3.11 (d, J=24.3 Hz, 3H), 2.84 (dd, J=4.8, 3.0 Hz, 3H), 2.16-1.95 (m, 4H), 0.84 (tt, J=5.9, 2.2 Hz, 4H)

TABLE 5
The following compounds were prepared under analogous conditions as
compound 33-2 in example 33:

| | | LCMS m/z | |
|---|---|---|---|
| Compound | Structure | (ES+) | ¹H NMR |
| 34 | 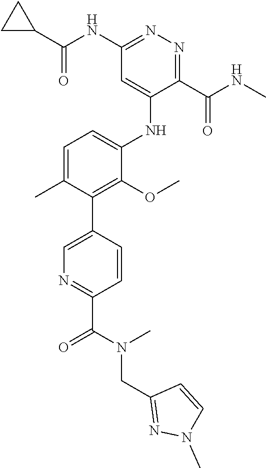 [SMILES: CNC(=O)c1nnc(NC(=O)C2CC2)cc1Nc1ccc(C)c(-c2ccc(C(=O)N(C)Cc3ccn(C)n3)nc2)c1OC] | 584.3 [M + H]+ | |
| 164 | 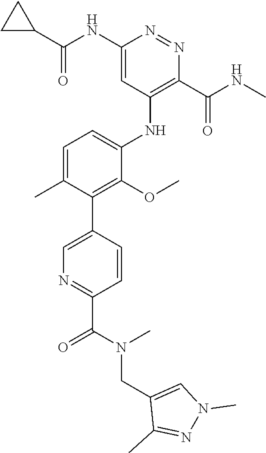 [SMILES: CNC(=O)c1nnc(NC(=O)C2CC2)cc1Nc1ccc(C)c(-c2ccc(C(=O)N(C)Cc3cn(C)nc3C)nc2)c1OC] | 598.2 [M + H]+ | ¹H NMR (500 MHz, DMSO) δ 11.30 (s, 1H), 10.80 (s, 1H), 9.12 (q, J = 4.7 Hz, 1H), 8.53 (dd, J = 25.5, 2.2 Hz, 1H), 8.11 (s, 1H), 7.90 (td, J = 8.4, 2.2 Hz, 1H), 7.66 (dd, J = 10.7, 8.0 Hz, 1H), 7.58 (d, J = 13.8 Hz, 1H), 7.41 (dd, J = 8.2, 2.1 Hz, 1H), 7.19 (dd, J = 8.5, 2.6 Hz, 1H), 4.47 (d, J = 29.5 Hz, 2H), 3.71 (d, J = 18.7 Hz, 3H), 3.29 (d, J = 1.1 Hz, 3H), 2.90 (d, J = 30.9 Hz, 3H), 2.84 (s, 3H), 2.08 (m, 4H), 1.98 (d, J = 131 Hz, 3H), 1.11- 0.75 (m, 4H). NMR is indicative of rotamers |
| 180 | 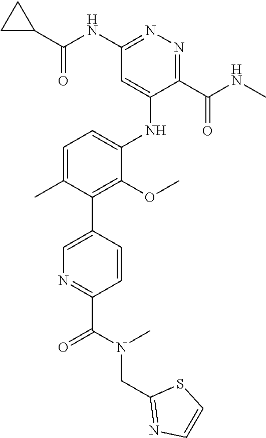 [SMILES: CNC(=O)c1nnc(NC(=O)C2CC2)cc1Nc1ccc(C)c(-c2ccc(C(=O)N(C)Cc3nccs3)nc2)c1OC] | 587.2 (M + H)+ | ¹H NMR (400 MHz, DMSO-d6) 11.32 (s, 1H), 10.80 (d, J = 6.1 Hz, 1H), 9.14 (s, 1H), 8.55 (d, J = 14.9 Hz, 1H), 8.12 (d, J = 6.5 Hz, 1H), 7.94 (td, J = 5.5, 2.7 Hz, 1H), 7.83-7.67 (m, 3H), 7.42 (dd, J = 8.2, 4.4 Hz, 1H), 7.21 (dd, J = 8.4, 4.0 Hz, 1H), 5.04 (d, J = 26.2 Hz, 2H), 3.29 (d, J = 13.8 Hz, 3H), 3.10 (d, J = 16.6 Hz, 3H), 2.84 (d, J = 4.8 Hz, 3H), 2.09 (d, J = 5.1 Hz, 4H), 0.83 (s, 4H). |
| 296 | 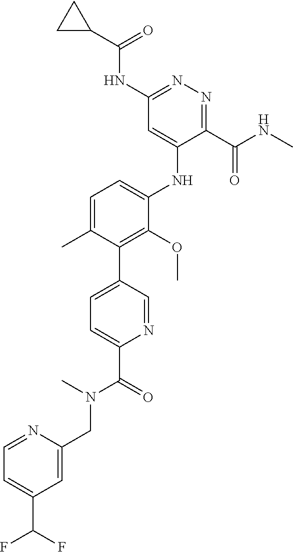 [SMILES: CNC(=O)c1nnc(NC(=O)C2CC2)cc1Nc1ccc(C)c(-c2ccc(C(=O)N(C)Cc3cc(C(F)F)ccn3)nc2)c1OC] | 631.3 (M + H)+ | ¹H NMR (400 MHz, DMSO-d6) 11.32 (d, J = 5.9 Hz, 1H), 10.81 (d, J = 13.7 Hz, 1H), 9.15 (d, J = 5.3 Hz, 1H), 8.72 (dd, J = 28.7, 4.9 Hz, 1H), 8.51 (d, J = 57.4 Hz, 1H), 8.13 (d, J = 12.2 Hz, 1H), 7.91 (dd, J = 38.7, 8.2 Hz, 1H), 7.71 (dd, J = 28.5, 8.1 Hz, 1H), 7.58-7.37 (m, 3H), 7.32- 6.99 (m, 2H), 4.90 (d, J = 8.0 Hz, 2H), 3.23 (s, 3H), 3.09 (d, J = 24.0 Hz, 3H), 2.84 (dd, J = 4.8, 3.1 Hz, 3H), 2.08 (d, J = 26.8 Hz, 4H), 0.83 (d, J = 5.7 Hz, 4H). |
| 317 | 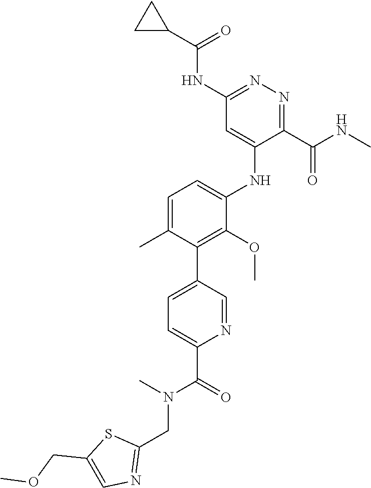 | 613.3 (M+H)+ | ¹H NMR (400 MHz, DMSO-d6) 11.32 (d, J = 2.1 Hz, 1H), 10.82 (d, J = 4.3 Hz, 1H), 9.14 (d, J = 5.3 Hz, 1H), 8.55 (ddd, J = 17.1, 2.1, 0.9 Hz, 1H), 8.13 (d, J = 4.8 Hz, 1H), 7.95 (ddd, J = 8.0, 4.3, 2.2 Hz, 1H), 7.75 (ddd, J = 8.8, 8.0, 0.9 Hz, 1H), 7.68 (dt, J = 18.1, 0.9 Hz, 1H), 7.42 (dd, J = 8.2, 3.9 Hz, 1H), 7.21 (dd, J = 8.4, 4.0 Hz, 1H), 5.00 (d, J = 29.7 Hz, 2H), 4.61 (dd, J = 17.0, 0.9 Hz, 2H), 3.33-3.23 (m, 6H), 3.10 (d, J = 19.3 Hz, 3H), 2.87- 2.81 (m, 3H), 2.10 (d, J = 4.9 Hz, 4H), 0.84 (q, J = 3.0 Hz, 4H). |
| 347 | 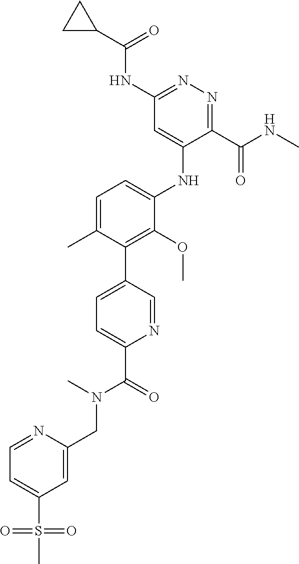 [SMILES: CNC(=O)c1nnc(NC(=O)C2CC2)cc1Nc1ccc(C)c(-c2ccc(C(=O)N(C)Cc3cc(S(C)(=O)=O)ccn3)nc2)c1OC] | 659.3 | ¹H NMR (400 MHz, DMSO-d6) 11.33 (d, J = 5.5 Hz, 1H), 10.81 (d, J = 12.5 Hz, 1H), 9.23-9.07 (m, 1H), 8.89 (dd, J = 27.4, 5.0 Hz, 1H), 8.62-8.38 (m, 1H), 8.13 (d, J = 11.2 Hz, 1H), 7.96 (dd, J = 7.9, 2.2 Hz, 1H), 7.90- 7.78 (m, 2H), 7.73 (dd, J = 12.6, 7.9 Hz, 1H), 7.42 (dd, J = 12.3, 8.2 Hz, 1H), 7.20 (dd, J = 14.5, 8.4 Hz, 1H), 4.97 (d, J = 10.5 Hz, 2H), 3.37 (s, 2H), 3.32 (d, J = 2.0 Hz, 4H), 3.24 (s, 1H), 3.16 (s, 1H), 3.08 (s, 1H), 2.84 (dd, J = 4.8, 3.3 Hz, 3H), 2.19-1.99 (m, 4H), 0.84 (s, 4H). |

### Example 35

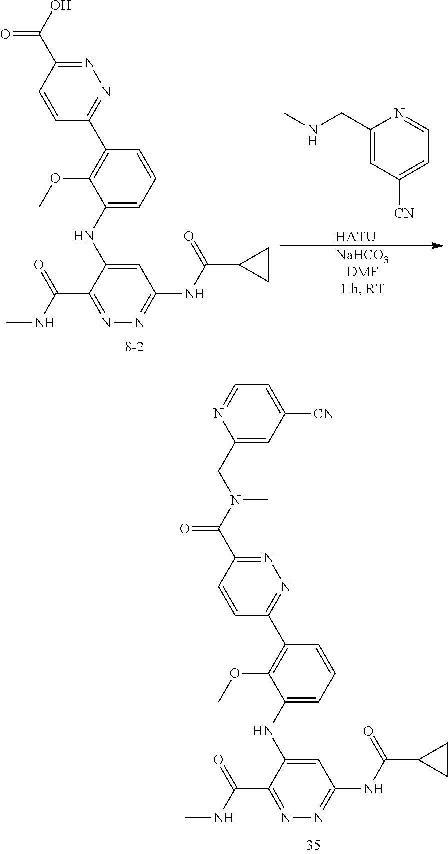

Compound 35 was prepared from acid 8-2 using the analogous coupling conditions from Example 11.

LC/MS (ES+) m/z=593.3 [M+H]

TABLE 6
The following compounds were prepared in analogous fashion to
compound 35-1 in Example 35:

| | | LCMS m/z | |
|---|---|---|---|
| Compound | Structure | (ES+) | ¹H NMR |
| 75 | 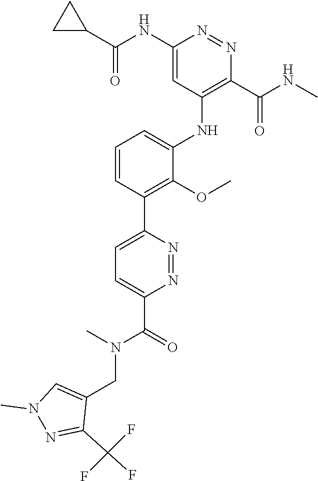 | 639.5 [M + H]+ | |
| 76 | 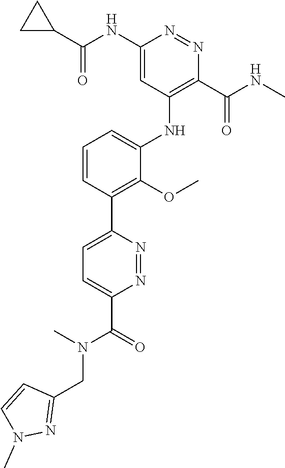 [SMILES: CNC(=O)c1nnc(NC(=O)C2CC2)cc1Nc1cccc(-c2ccc(C(=O)N(C)Cc3ccn(C)n3)nn2)c1OC] | 571.4 [M + H]+ | |

### Example 36

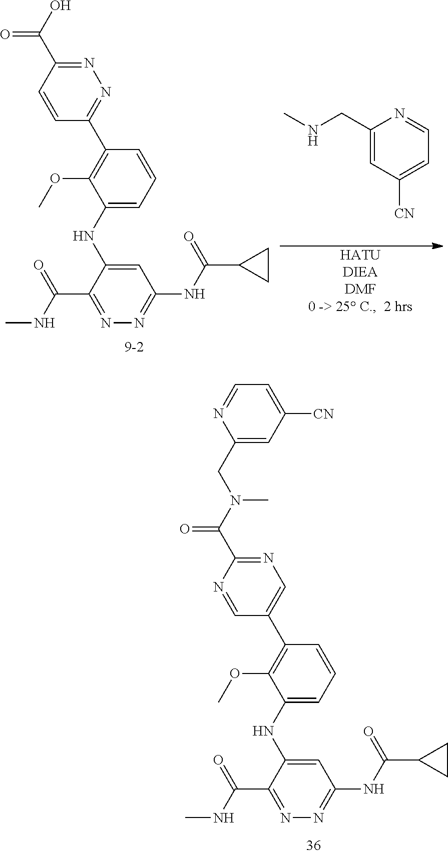

Compound 36 was prepared from acid 9-2 using the analogous coupling conditions from Example 18.

LCMS m/z (ES+): 593. [M+H]+

TABLE 7
The following compounds were prepared under analogous conditions as
compound 36 in example 36:

| | | LCMS m/z | |
|---|---|---|---|
| Compound | Structure | (ES+) | ¹H NMR |
| 49 | 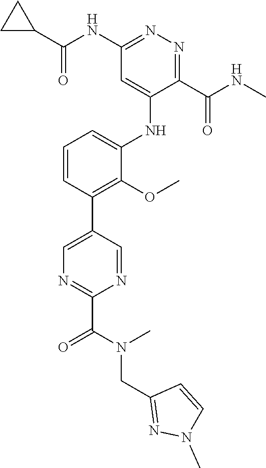 [SMILES: CNC(=O)c1nnc(NC(=O)C2CC2)cc1Nc1cccc(-c2cnc(C(=O)N(C)Cc3ccn(C)n3)nc2)c1OC] | 571.3 [M + H]+ | |
| 56 | 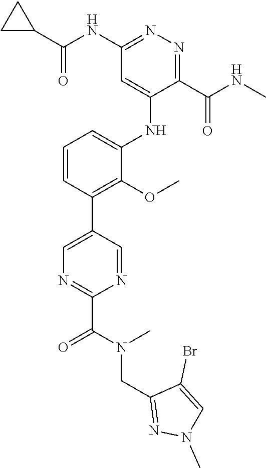 [SMILES: CNC(=O)c1nnc(NC(=O)C2CC2)cc1Nc1cccc(-c2cnc(C(=O)N(C)Cc3nn(C)cc3Br)nc2)c1OC] | 651.2 [M + H]+ | |
| 64 | 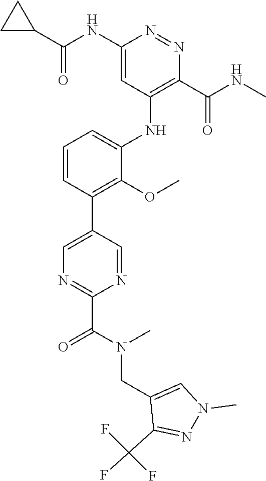 [SMILES: CNC(=O)c1nnc(NC(=O)C2CC2)cc1Nc1cccc(-c2cnc(C(=O)N(C)Cc3cn(C)nc3C(F)(F)F)nc2)c1OC] | 639.4 [M + H]+ | ¹H NMR (400 MHz, DMSO) δ 11.35 (s, 1H), 10.98 (d, J = 2.9 Hz, 1H), 9.18 (d, J = 5.0 Hz, 1H), 9.10 (s, 1H), 9.08 (s, 1H), 8.16 (d, J = 3.2 Hz, 1H), 7.92 (s, 1H), 7.64-7.52 (m, 1H), 7.45- 7.31 (m, 2H), 4.52 (d, J = 106.2 Hz, 2H), 3.91 (d, J = 11.6 Hz, 3H), 3.42 (d, J = 10.8 Hz, 3H), 2.85 (d, J = 4.8 Hz, 3H), 2.80 (s, 3H), 2.14-2.06 (m, 1H), 0.83 (d, J = 8.5 Hz, 4H). |

### Example 37

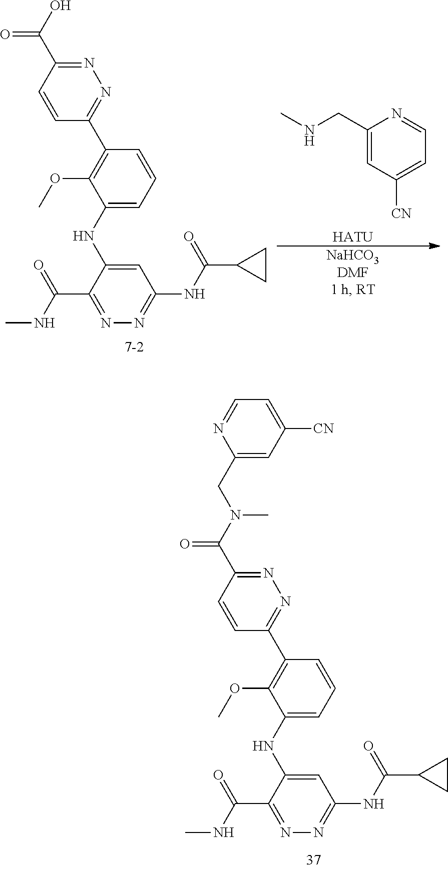

Compound 37 was prepared from acid 7-2 using the analogous coupling conditions from Example 11.

LCMS m/z (ES+): 593.4 [M+H]+

### Example 38

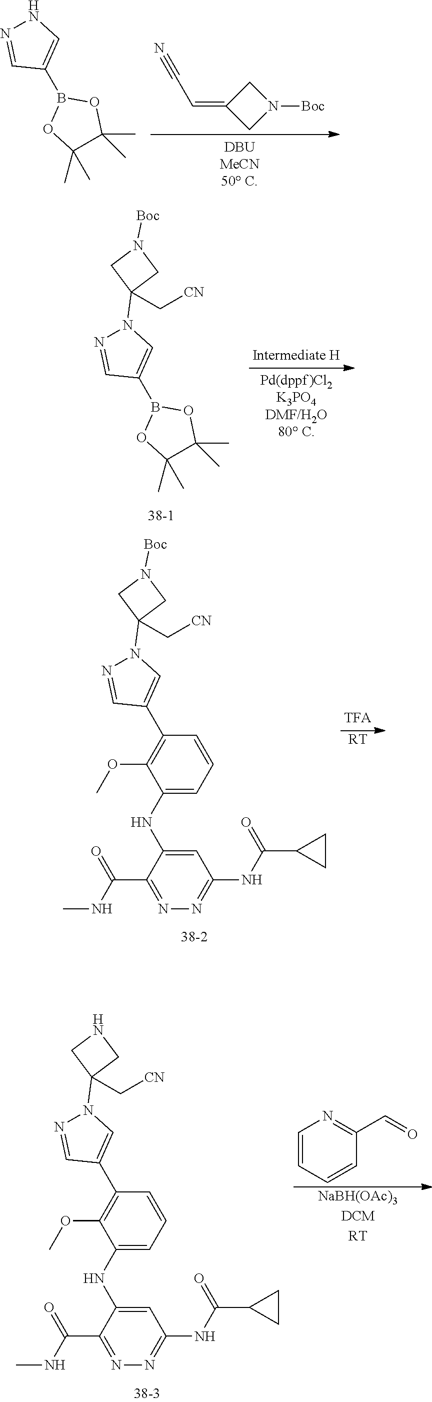

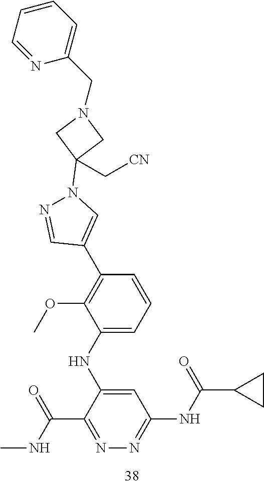 [SMILES: CNC(=O)c1nnc(NC(=O)C2CC2)cc1Nc1cccc(-c2cnn(C3(CC#N)CN(Cc4ccccn4)C3)c2)c1OC]

Step 1: To a solution of 4-(4,4,5,5-tetramethyl-1,3,2-dioxaborolan-2-yl)-1H-pyrazole (1.99 g, 1.0 equiv, 10.3 mmol) in acetonitrile (30 mL) was added tert-butyl 3-(cyanomethylene) azetidine-1-carboxylate (1.99 g, 1.0 equiv, 10.3 mmol) followed by 1,8-diazabicyclo[5.4.0] undec-7-ene (1.56 g, 1.53 mL, 1.0 equiv, 10.3 mmol). The mixture was then stirred at 50° C. for 16 hours, concentrated down, and purified by silica gel chromatography with a (gradient: ethyl acetate/heptanes) to provide compound 38-1 (2.3 g, 5.97 mmol, 58% yield) as a white solid.

LCMS m/z (ES+): 333.3 [M+H-56]⁺

Step 2: In a sealed tube, a mixture of Intermediate H (0.88 g, 1.0 equiv, 2.09 mmol), compound 38-1 (1.16 g, 1.43 equiv, 2.99 mmol), Pd(dppf)Cl₂ (230 mg, 0.1500 Eq, 314 μmol), and potassium phosphate, tribasic (888 mg, 346 μL, 2.00 equiv, 4.18 mmol) in DMF (7.0 mL) and Water (2.3 mL) was degassed with nitrogen. The tube was sealed, and the mixture was stirred for 16 hours at 75-80° C. until the reaction was determined to be complete by LC/MS analysis. The reaction was then treated with water (˜70 mL). The resulting dark yellow solid was isolated by filtration and dried. The isolated solid was treated with MeOH (˜7 mL). The filtrate was concentrated down and purified by silica gel chromatography (gradient: DCM to 10:90:0.5 MeOH/DCM/NH₄OH) to yield compound 38-2 (135.4 mg, 225.0 μmol, 11%) as a yellow, foamy solid.

LCMS m/z (ES+): 602.6 [M+H]⁺

Step 3: A solution of compound 38-2 (135.4 mg, 1.0 equiv, 225.0 μmol) in DCM (3.5 mL) and trifluoroacetic acid (1.283 g, 855.9 μL, 50 equiv, 11.25 mmol) was stirred at RT for 1 hour. The ensuing green solution was concentrated down and dried to yield 300 mg of crude TFA salt of compound 38-3 that was used directly in the next step.

LCMS m/z (ES+): 502.3 [M+H]⁺.

Step 4: A mixture of compound 38-3 (91.5 mg, 1.0 equiv, 182 μmol) and picolinaldehyde (23.4 mg, 1.2 equiv, 219 μmol) in DCM (2.5 mL) was stirred 15 minutes at RT followed by the slow addition over 30 minutes of sodium triacetoxyborohydride (77.3 mg, 2.0 equiv, 365 μmol). The reaction was then stirred for 20 min until the starting material was determined to be consumed by LC/MS analysis. The reaction was then treated with DCM (30 mL) and saturated aqueous NaHCO₃ (10 mL). The layers were separated and the organic layer was dried over sodium sulfate, filtered, concentrated down, and purified by silica gel chromatography (gradient: DCM to 10:90:0.5 MeOH/DCM/NH₄OH) to provide compound 38. (49.5 mg, 79 μmol, 43%, yield) as a light yellow, foamy solid.

LCMS m/z (ES+): 593.4 [M+H]⁺

¹H NMR (500 MHZ, DMSO) § 11.29 (s, 1H), 11.00 (s, 1H), 9.14 (q, J=4.8 Hz, 1H), 8.51-8.48 (m, 1H), 8.46 (s, 1H), 8.15 (s, 1H), 8.07 (s, 1H), 7.76 (td, J=7.6, 1.8 Hz, 1H), 7.49 (dd, J=7.8, 1.5 Hz, 1H), 7.40 (d, J=7.8 Hz, 1H), 7.31 (dd, J=8.0, 1.6 Hz, 1H), 7.26 (ddd, J=7.5, 4.8, 1.2 Hz, 1H), 7.21 (t, J=7.9 Hz, 1H), 3.84 (s, 2H), 3.78 (d, J=8.0 Hz, 2H), 3.68-3.64 (m, 2H), 3.58 (s, 3H), 3.52 (s, 2H), 2.85 (d, J=4.8 Hz, 3H), 2.07 (p, J=6.3 Hz, 1H), 0.86-0.75 (m, 4H).

### Example 42

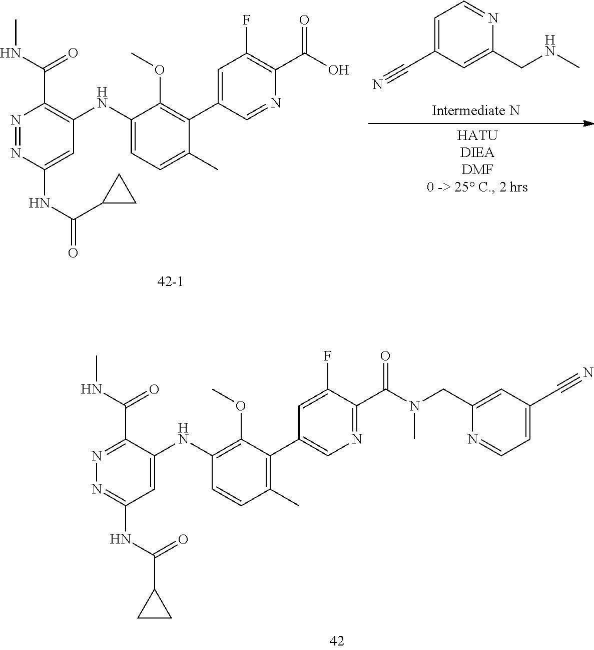

Compound 42-1 was prepared in analogous fashion to Intermediate M except that Intermediate I was used in place of Intermediate H and tert-butyl 5-bromo-3-fluoropicolinate was used in place of tert-butyl 5-bromo-picolinate. Compound 42-1 was then converted to desired amide 42 using the coupling conditions in Example 18.

LCMS m/z (ES+): 624.4 [M+H]+

TABLE 8
The following compounds were prepared under analogous conditions as
compound 42 in example 42:

| | | LCMS | |
|---|---|---|---|
| Compound | Structure | m/z (ES+) | 1H NMR |
| 43 | 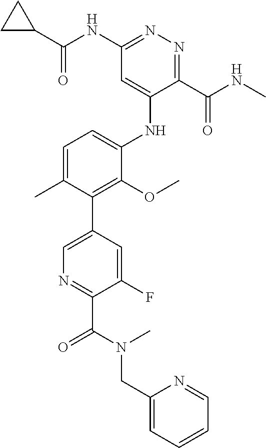 [SMILES: CNC(=O)c1nnc(NC(=O)C2CC2)cc1Nc1ccc(C)c(-c2cnc(C(=O)N(C)Cc3ccccn3)c(F)c2)c1OC] | 599.5 [M + H]+ | |
| 47 | 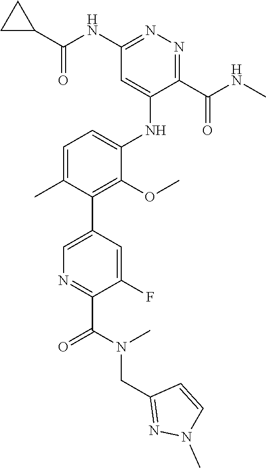 [SMILES: CNC(=O)c1nnc(NC(=O)C2CC2)cc1Nc1ccc(C)c(-c2cnc(C(=O)N(C)Cc3ccn(C)n3)c(F)c2)c1OC] | 602.5 [M + H]+ | |
| 117* | 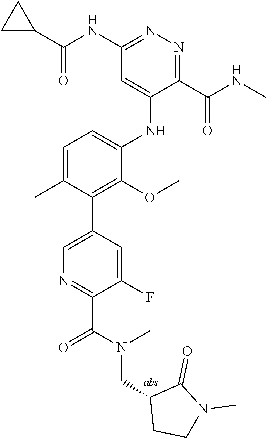 [SMILES: CNC(=O)c1nnc(NC(=O)C2CC2)cc1Nc1ccc(C)c(-c2cnc(C(=O)N(C)C[C@H]3CCN(C)C3=O)c(F)c2)c1OC] | 619.4 [M + H]+ | ¹H NMR (400 MHz, Chloroform-d) 10.90 (d, J = 3.1 Hz, 1H), 9.74 (d, J = 23.0 Hz, 1H), 8.46-8.38 (m, 1H), 8.19 (d, J = 2.6 Hz, 1H), 8.09 (q, J = 5.1 Hz, 1H), 7.61-7.53 (m, 1H), 7.40 (d, J = 8.2 Hz, 1H), 7.16 (d, J = 8.3 Hz, 1H), 4.18-4.08 (m, 1H), 3.86-3.75 (m, 1H), 3.50-3.43 (m, 1H), 3.38 (d, J = 1.5 Hz, 3H), 3.35-3.22 (m, 1H), 3.20 (s, 1H), 3.05 (d, J = 5.0 Hz, 3H), 3.02 (s, 2H), 2.91 (s, 2H), 2.91-2.81 (m, 1H), 2.81 (s, 1H), 2.36-2.21 (m, 1H), 2.17 (d, J = 3.8 Hz, 3H), 2.00 (s, 1H), 1.91- 1.81 (m, 1H), 1.23-1.10 (m, 2H), 0.99-0.89 (m, 2H) |
| 230 | 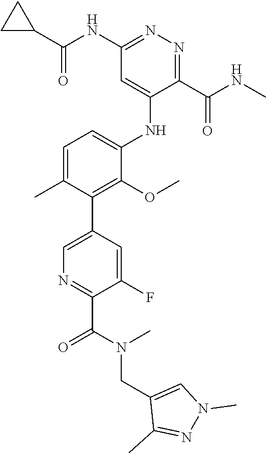 [SMILES: CNC(=O)c1nnc(NC(=O)C2CC2)cc1Nc1ccc(C)c(-c2cnc(C(=O)N(C)Cc3cn(C)nc3C)c(F)c2)c1OC] | 616.4 [M + H]+ | ¹H NMR (400 MHz, Chloroform-d) 11.12 (s, 1H), 9.53 (s, 1H), 8.44 (s, 1H), 8.18 (s, 1H), 8.05 (s, 1H), 7.59 (d, J = 9.6 Hz, 1H), 7.45-7.37 (m, 2H), 7.18 (d, J = 8.1 Hz, 1H), 4.65 (s, 1H), 4.32 (s, 1H), 3.85 (d, J = 4.4 Hz, 3H), 3.38 (d, J = 1.5 Hz, 3H), 3.11-3.03 (m, 4H), 2.90 (s, 2H), 2.34 (s, 2H), 2.21- 2.15 (m, 4H), 1.78 (s, 1H), 1.15 (t, J = 3.6 Hz, 2H), 1.03-0.97 (m, 2H). |
| 289 | 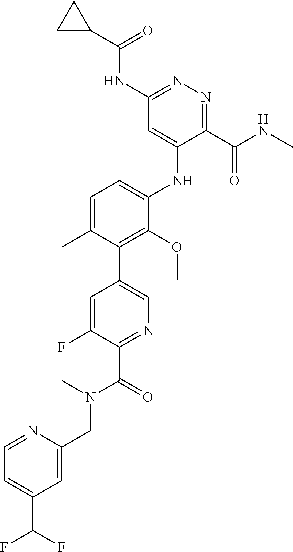 [SMILES: CNC(=O)c1nnc(NC(=O)C2CC2)cc1Nc1ccc(C)c(-c2cnc(C(=O)N(C)Cc3cc(C(F)F)ccn3)c(F)c2)c1OC] | 649.3 [M + H]+ | ¹H NMR (400 MHz, DMSO-d6) 11.33 (d, J = 4.9 Hz, 1H), 10.79 (d, J = 11.0 Hz, 1H), 9.15 (d, J = 5.1 Hz, 1H), 8.72 (dd, J = 40.2, 5.1 Hz, 1H), 8.51-8.29 (m, 1H), 8.11 (d, J = 8.9 Hz, 1H), 8.06-7.80 (m, 1H), 7.54 (s, 1H), 7.50 ^{‥}C. 7.39 (m, 2H), 7.32-6.93 (m, 2H), 4.81 (d, J = 83.2 Hz, 2H), 3.34 (s, 3H), 3.27 (s, 1H), 3.06 (d, J = 52.9 Hz, 3H), 2.85 (dd, J = 4.8, 2.5 Hz, 3H), 2.11 (d, J = 24.8 Hz, 4H), 0.84 (d, J = 4.4 Hz, 4H). |
| 314 | 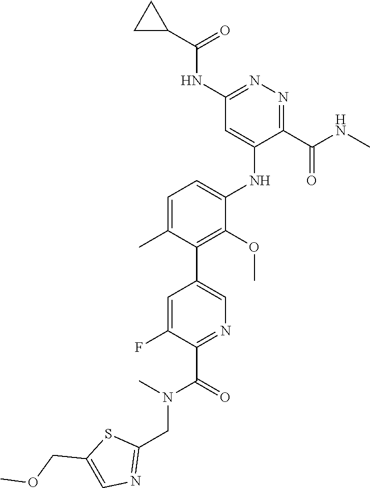 | 649.3 [M + H]+ | ¹H NMR (400 MHz, DMSO- d6) 11.33 (s, 1H), 10.80 (d, J = 4.1 Hz, 1H), 9.18-9.12 (m, 1H), 8.48 (t, J = 1.6 Hz, 1H), 8.11 (d, J = 3.2 Hz, 1H), 7.98 (ddd, J = 22.6, 10.3, 1.6 Hz, 1H), 7.71 (d, J = 0.9 Hz, 1H), 7.44 (dd, J = 8.2, 4.6 Hz, 1H), 7.22 (dd, J = 8.4, 5.1 Hz, 1H), 4.91 (d, J = 73.7 Hz, 2H), 4.61 (dd, J = 19.9, 0.9 Hz, 2H), 3.34 (s, 3H), 3.30 (d, J = 8.6 Hz, 3H), 3.06 (d, J = 53.2 Hz, 3H), 2.88-2.82 (m, 3H), 2.16-2.04 (m, 4H), 0.88- 0.80 (m, 4H). |
| 340 | 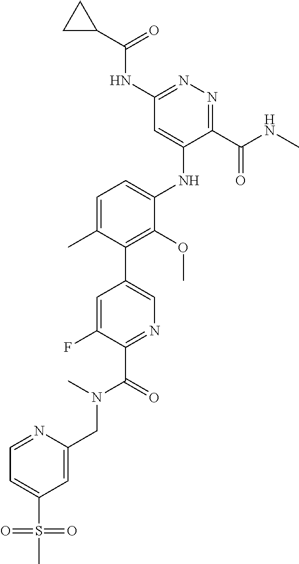 [SMILES: CNC(=O)c1nnc(NC(=O)C2CC2)cc1Nc1ccc(C)c(-c2cnc(C(=O)N(C)Cc3cc(S(C)(=O)=O)ccn3)c(F)c2)c1OC] | 677.3 [M + H]+ | ¹H NMR (400 MHz, DMSO-d6) 11.33 (d, J = 4.5 Hz, 1H), 10.79 (d, J = 9.9 Hz, 1H), 9.15 (t, J = 4.4 Hz, 1H), 8.97-8.81 (m, 1H), 8.41 (dt, J = 58.5, 1.6 Hz, 1H), 8.16-7.96 (m, 2H), 7.92-7.73 (m, 2H), 7.43 (dd, J = 11.2, 8.2 Hz, 1H), 7.21 (dd, J = 13.5, 8.3 Hz, 1H), 4.88 (d, J = 82.4 Hz, 2H), 3.36 (s, 3H), 3.29 (d, J = 9.9 Hz, 3H), 3.08 (d, J = 45.8 Hz, 3H), 2.85 (dd, J = 4.8, 2.6 Hz, 3H), 2.12 (d, J = 23.7 Hz, 4H), 0.84 (dq, J = 6.6, 4.1 Hz, 4H). |

*Absolute stereochemistry arbitrarily assigned.

### Example 55

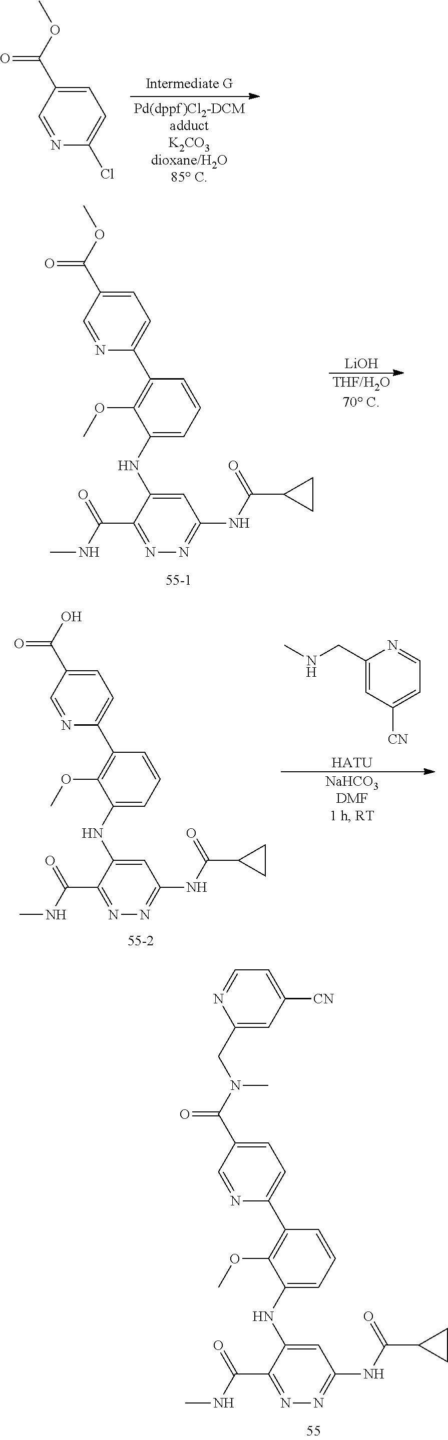

Step 1: A round bottomed flask was charged with Intermediate G (150 mg, 1.0 equiv, 321 μmol), methyl 6-chloronicotinate (55.1 mg, 1.0 equiv, 321 μmol), K₂CO₃ (133 mg, 3.0 equiv, 963 μmol), PdCl₂(dppf)-CH₂Cl₂ adduct (52.4 mg, 0.2 equiv, 64.2 μmol). 1,4-Dioxane (2 mL) and water (0.4 mL) were added, and the solution was stirred at 85° C. for 2 hour under N₂ after which the reaction was quenched with water and extracted with ethyl acetate. The organic layer was washed with brine, dried over Na₂SO₄ and evaporated. The crude product was then purified by prep-TLC (ethyl acetate/petroleum ether=1:1) to provide compound 55-1 (80 mg, 0.17 mmol, 52%) as a white solid.

LC/MS (ES+): 477.1 [M+H]+

Step 2: A round bottomed flask was charged with compound 55-1 (65 mg, 1.0 equiv, 0.14 mmol) in THF (1 mL), LiOH (3.3 mg, 1.0, 0.14 mmol) and water (1 mL) was added, after which the solution was stirred at 70° C. for 30 min. The reaction was quenched with 1M HCl and extracted with ethyl acetate. The organic layer was dried over Na₂SO₄ and evaporated. The crude product was then purified with prep-TLC (ethyl acetate/petroleum ether=1:1 v/v) to provide acid 55-2 (30 mg, 65 μmol, 48% yield) as a white solid.

LC/MS (ES+): 463.15 [M+H]+

Step 3: A round bottomed flask was charged with acid 55-2 (25 mg, 1.0 equiv, 54 μmol), 2-((methylamino)methyl) isonicotinonitrile (16 mg, 2.0 equiv, 0.11 mmol), HATU (31 mg, 1.5 equiv, 81 μmol), and sodium bicarbonate (23 mg, 5.0 equiv, 0.27 mmol). DMF (2 mL) was added, and the solution was stirred at 25° C. for 2 hour. The resulted solution was purified using C18 flash chromatography with the following conditions (gradient: water/MeCN). Lyophilization of product-containing fractions provided compound 55 (13.4 mg, 22.6 μmol, 42% yield) as a white amorphous solid.

LC/MS (ES+): 592.25 [M+H]+

¹H NMR (400 MHZ, DMSO-d6) 11.33 (s, 1H), 10.98 (d, J=13.2 Hz, 1H), 9.17 (s, 1H), 8.94-8.72 (m, 2H), 8.23-7.75 (m, 5H), 7.65-7.46 (m, 2H), 7.34 (t, J=8.1 Hz, 1H), 4.88 (s, 1H), 4.71 (s, 1H), 3.49 (d, J=20.4 Hz, 3H), 3.05 (d, J=47.4 Hz, 3H), 2.87 (d, J=4.8 Hz, 3H), 2.09 (t, J=6.2 Hz, 1H), 0.83 (d, J=7.0 Hz, 4H).

### Example 61

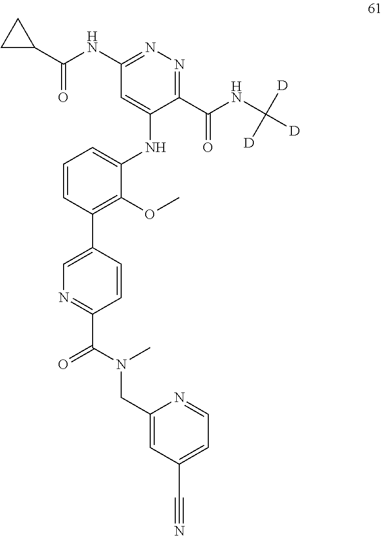 [SMILES: [2H]C([2H])([2H])NC(=O)c1nnc(NC(=O)C2CC2)cc1Nc1cccc(-c2ccc(C(=O)N(C)Cc3cc(C#N)ccn3)nc2)c1OC]

Compound 61 was prepared in analogous fashion to the analogues in Example 18 except that Intermediate B was used as starting material.

LC/MS (ES+): 595.5 [M+H]+

TABLE 9
The following compounds were prepared in analogous fashion to compounds
in example 18 except that Intermediate B was used as starting material:

| | | LCMS m/z | |
|---|---|---|---|
| Compound | Structure | (ES+) | 1H NMR |
| 74 | 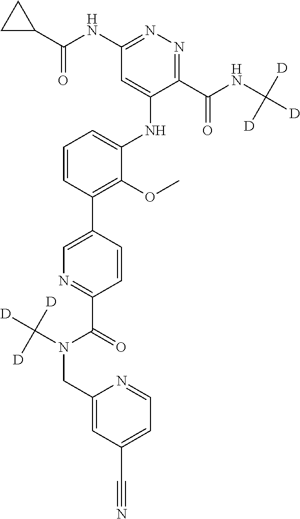 [SMILES: [2H]C([2H])([2H])NC(=O)c1nnc(NC(=O)C2CC2)cc1Nc1cccc(-c2ccc(C(=O)N(Cc3cc(C#N)ccn3)C([2H])([2H])[2H])nc2)c1OC] | 598.4 (M + H)+ | |
| 79 | 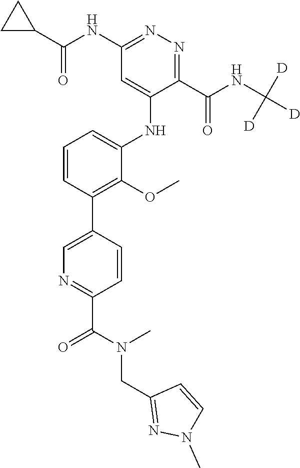 [SMILES: [2H]C([2H])([2H])NC(=O)c1nnc(NC(=O)C2CC2)cc1Nc1cccc(-c2ccc(C(=O)N(C)Cc3ccn(C)n3)nc2)c1OC] | 573.4 | |

### Example 62

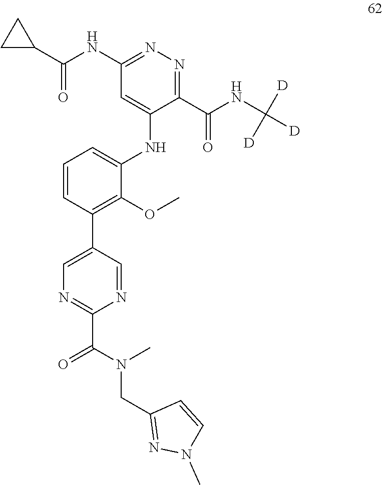 [SMILES: [2H]C([2H])([2H])NC(=O)c1nnc(NC(=O)C2CC2)cc1Nc1cccc(-c2cnc(C(=O)N(C)Cc3ccn(C)n3)nc2)c1OC]

Compound 62 was was prepared in analogous fashion to the analogues in Example 36 except that Intermediate B was used as starting material.

LC/MS (ES+): 574.5 [M+H]+

### Example 63

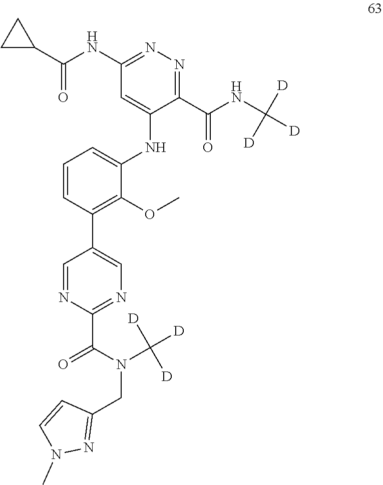 [SMILES: [2H]C([2H])([2H])NC(=O)c1nnc(NC(=O)C2CC2)cc1Nc1cccc(-c2cnc(C(=O)N(Cc3ccn(C)n3)C([2H])([2H])[2H])nc2)c1OC]

Compound 63 was was prepared in analogous fashion to the analogues in Example 36 except that Intermediate B was used as starting material.

LC/MS (ES+): 577.4 [M+H]+

### Example 68

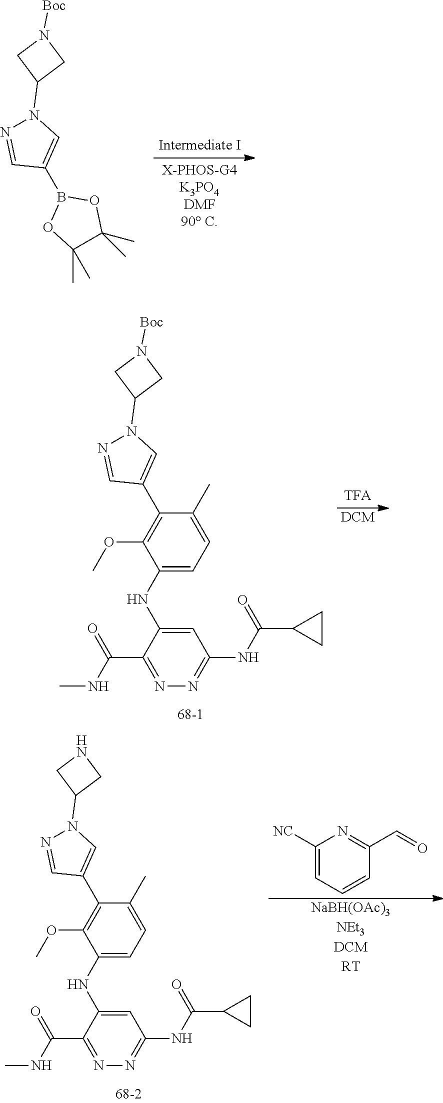

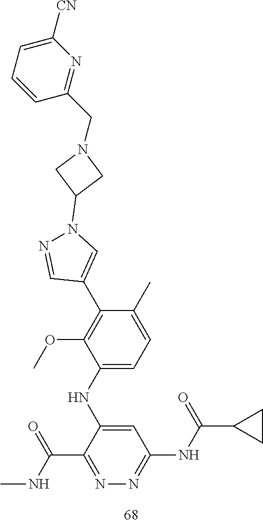 [SMILES: CNC(=O)c1nnc(NC(=O)C2CC2)cc1Nc1ccc(C)c(-c2cnn(C3CN(Cc4cccc(C#N)n4)C3)c2)c1OC]

Step 1: In a sealed tube degassed with nitrogen, a mixture of Intermediate I (406.7 mg, 1.0 equiv, 936.5 μmol), tert-butyl 3-(4-(4,4,5,5-tetramethyl-1,3,2-dioxaborolan-2-yl)-1H-pyrazol-1-yl) azetidine-1-carboxylate (588.7 mg, 1.8 equiv, 1.686 mmol), (s)-(dicyclohexyl (2′,4′,6′-triisopropyl-[1,1′-biphenyl]-2-yl)-15-phosphaneyl) (2′-(methylamino)-[1,1′-biphenyl]-2-yl) palladium (III) methanesulfonate (X-PHOS-G4, 121.0 mg, 0.15 equiv, 140.5 μmol), and potassium phosphate (477.1 mg, 2.4 equiv, 2.248 mmol) in DMF (4.0 mL) and water (1.3 mL) was stirred at 90° C. for 2 hours. The reaction mixture was then cooled to RT and treated with water (˜25 mL) after which the resultant crude yellow solid was isolated by filtration and dried overnight. The crude material was purified by silica gel chromatography (gradient: DCM/10:90:0.5 MeOH/DCM/NH₄OH) to provide compound 68-1 (343.6 mg, 0.57 mmol, 60% yield) as a yellow, foamy solid.

LC/MS (ES+) m/z: 577.4 [M+H]+.

Step 2: A solution of compound 68-1 (343.6 mg, 95% Wt, 1.0 equiv, 566.1 μmol) in DCM (2 mL) and trifluoroacetic acid (1.936 g, 1.292 mL, 30 Eq, 16.98 mmol) was stirred at RT for 1 hour. The ensuing green solution was concentrated down and dried to yield crude TFA salt of compound 68-2, assumed to be quantitative (463.4 mg free base, 0.566 mmol). The product is a thick amber oil and used directly in the next step

LC/MS (ES+) m/z: 477.3 [M+H]+.

Step 3: A mixture of compound 68-2, 3TFA salt (304 mg, 54% Wt, 1.0 equiv, 201 μmol), 2-formylisonicotinonitrile (31.8 mg, 1.20 equiv, 241 μmol), and triethylamine (81.2 mg, 112 μL, 4.0 equiv, 802 μmol) in DCM (1.2 mL) was stirred 20 minutes at RT followed by the slow addition of sodium triacetoxyborohydride (85.0 mg, 2.0 equiv, 401 μmol) over 20 minutes. About 20 minutes after addition of reducing agent was completed, the reaction was diluted with DCM (10 mL) and treated with saturated aqueous NaHCO₃ (5 mL). The layers were separated, and the organic layer was dried over sodium sulfate, filtered, concentrated down, and dried. The crude was purified by silica gel chromatography with a (gradient: DCM/10:90:0.5 MeOH/DCM/NH₄OH) to yield compound 68 (86.1 mg, 0.14 mmol, 72% yield) as a light yellow solid.

LC/MS (ES+) m/z: 593.4 [M+H]+

¹H NMR (400 MHZ, DMSO) δ 11.27 (s, 1H), 10.79 (s, 1H), 9.11 (d, J=4.9 Hz, 1H), 8.76 (dd, J=5.0, 0.9 Hz, 1H), 8.13-8.04 (m, 2H), 7.83-7.78 (m, 1H), 7.74 (dd, J=5.0, 1.6 Hz, 1H), 7.68-7.63 (m, 1H), 7.25 (d, J=8.2 Hz, 1H), 7.10 (d, J=8.3 Hz, 1H), 5.11 (p, J=7.0 Hz, 1H), 3.91 (s, 2H), 3.85 (t, J=7.4 Hz, 2H), 3.66-3.57 (m, 2H), 3.34 (s, 3H), 2.83 (d, J=4.8 Hz, 3H), 2.24 (s, 3H), 2.07 (p, J=7.1 Hz, 1H), 0.86-0.76 (m, 4H).

TABLE 10
The following compounds were prepared in analogous fashion to the
procedures in example 68:

| | | LCMS m/z | |
|---|---|---|---|
| Compound | Structure | (ES+) | 1H NMR |
| 67 | 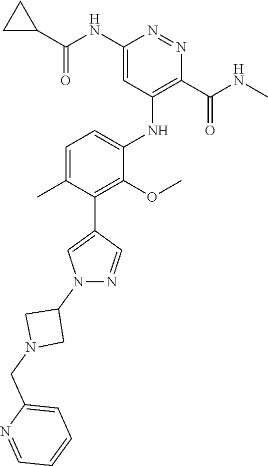 [SMILES: CNC(=O)c1nnc(NC(=O)C2CC2)cc1Nc1ccc(C)c(-c2cnn(C3CN(Cc4ccccn4)C3)c2)c1OC] | 568.5 [M + H]+ | ¹H NMR (400 MHz, DMSO) δ 11.27 (s, 1H), 10.79 (s, 1H), 9.15- 9.08 (m, 1H), 8.49 (ddd, J = 4.9, 1.9, 1.0 Hz, 1H), 8.08 (s, 1H), 8.06 (d, J = 0.8 Hz, 1H), 7.75 (td, J = 7.7, 1.9 Hz, 1H), 7.65 (d, J = 0.7 Hz, 1H), 7.40 (dt, J = 7.9, 1.1 Hz, 1H), 7.27-7.23 (m, 2H), 7.10 (d, J = 8.3 Hz, 1H), 5.09 (p, J = 6.9 Hz, 1H), 3.85-3.77 (m, 4H), 3.57 (td, J = 6.7, 1.6 Hz, 2H), 3.33 (s, 3H), 2.83 (d, J = 4.8 Hz, 3H), 2.23 (s, 3H), 2.11-2.02 (m, 1H), 0.84- 0.76 (m, 4H). |

### Example 87

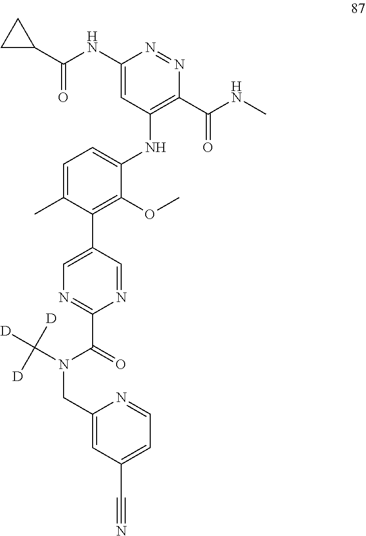 [SMILES: [2H]C([2H])([2H])N(Cc1cc(C#N)ccn1)C(=O)c1ncc(-c2c(C)ccc(Nc3cc(NC(=O)C4CC4)nnc3C(=O)NC)c2OC)cn1]

Compound 87 was was prepared in analogous fashion to the analogues in Example 36 except that Intermediate B was used as starting material.

LC/MS (ES+) m/z: 610.4 [M+H]+

### Example 92

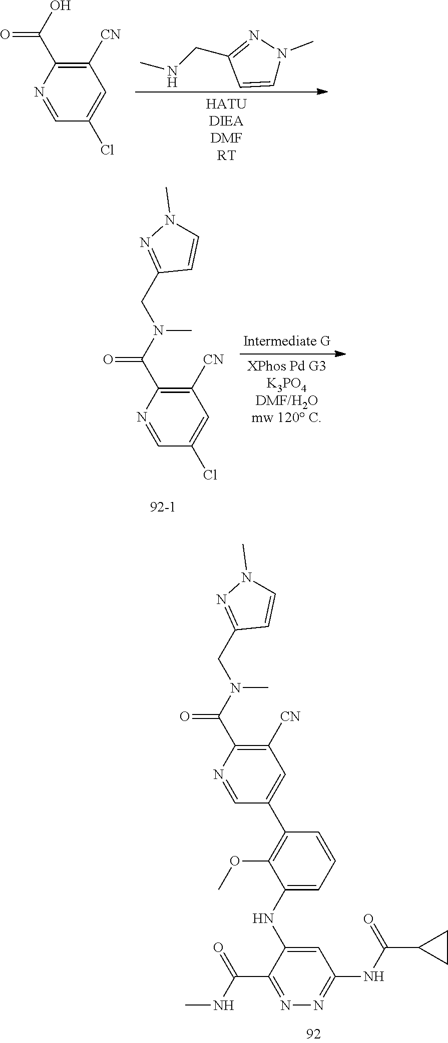

Step 1: A screw cap vial was loaded with HATU (542 mg, 1.3 equiv, 1.42 mmol), 5-chloro-3-cyanopicolinic acid (200 mg, 1.0 equiv, 1.10 mmol) and N-methyl-1-(1-methyl-1H-pyrazol-3-yl) methanamine (206 mg, 1.5 Eq, 1.64 mmol) followed the addition of DMF (3 mL). DIPEA (283 mg, 382 μL, 2.0 equiv, 2.19 mmol). was then added and the reaction was stirred for 1 h until it was determined to be complete by LC/MS analysis. The reaction was then filtered and purified by flash high pressure chromatography on (C18 column, gradient MeCN/0.1% formic acid in water). The product containing fractions were concentrated yielding the 92-1 as an glassy oil.

LC/MS (ES+) m/z: 290.0 [M+H]+

¹H NMR (400 MHZ, DMSO) δ 8.97-8.90 (m, 1H), 8.74 (dd, J=11.7, 2.3 Hz, 1H), 7.63 (dd, J=23.2, 2.2 Hz, 1H), 6.14 (dd, J=31.7, 2.2 Hz, 1H), 4.49 (d, J=109.7 Hz, 2H), 3.78 (d, J=25.0 Hz, 3H), 2.91 (d, J=76.5 Hz, 3H). The NMR spectrum is indicative of rotamers.

Step 2: A screw cap vial was loaded with compound 92-1 (19 mg, 1.5 equiv, 64 μmol), potassium phosphate (23 mg, 2.5 equiv, 0.11 mmol), Intermediate G (20 mg, 1.0 equiv, 43 μmol) and XPhos Pd G3 (5.4 mg, 0.15 equiv, 6.4 μmol). The vial was degassed with nitrogen and filled with DMF (700 μL) and Water (233 μL) and the reaction was heated at 120° C. on the uwave for 15 min until the reaction was determined to be complete by LC/MS analysis. The reaction mixture was concentrated, and the residue was purified by flash silica gel chromatography (gradient: MeOH:DCM+0.1% NH₄OH). The product containing fractions were concentrated yielding the desired product as a pale-yellow solid.

LC/MS (ES+) m/z=595.4 [M+H]+

### Example 103

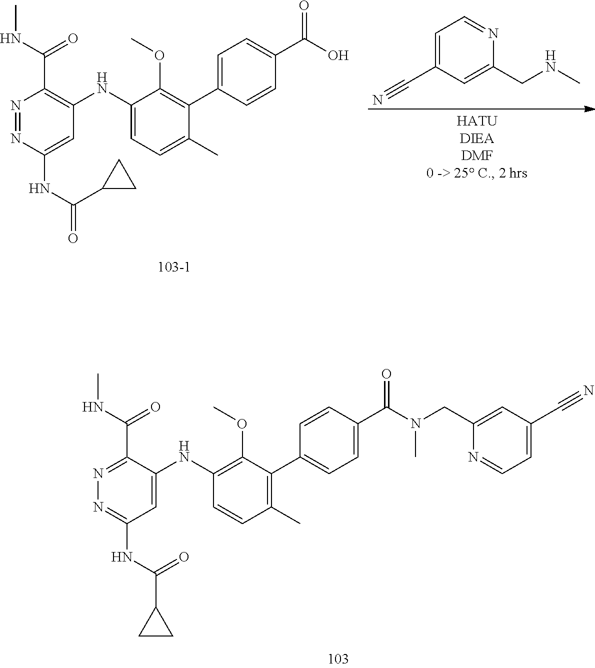 [SMILES: CNC(=O)c1nnc(NC(=O)C2CC2)cc1Nc1ccc(C)c(-c2ccc(C(=O)O)cc2)c1OC ; CNCc1cc(C#N)ccn1 ; CNC(=O)c1nnc(NC(=O)C2CC2)cc1Nc1ccc(C)c(-c2ccc(C(=O)N(C)Cc3cc(C#N)ccn3)cc2)c1OC]

Compound 103-1 was prepared in analogous fashion to compound 11-3 in Example 11 except that Intermediate I was used in place of Intermediate H. Intermediate N was then coupled to compound 103-1 to provide compound 103 using analogous procedures to those in Example 18.

LC/MS m/z (ES+): 605.4 [M+H]+

### Example 105

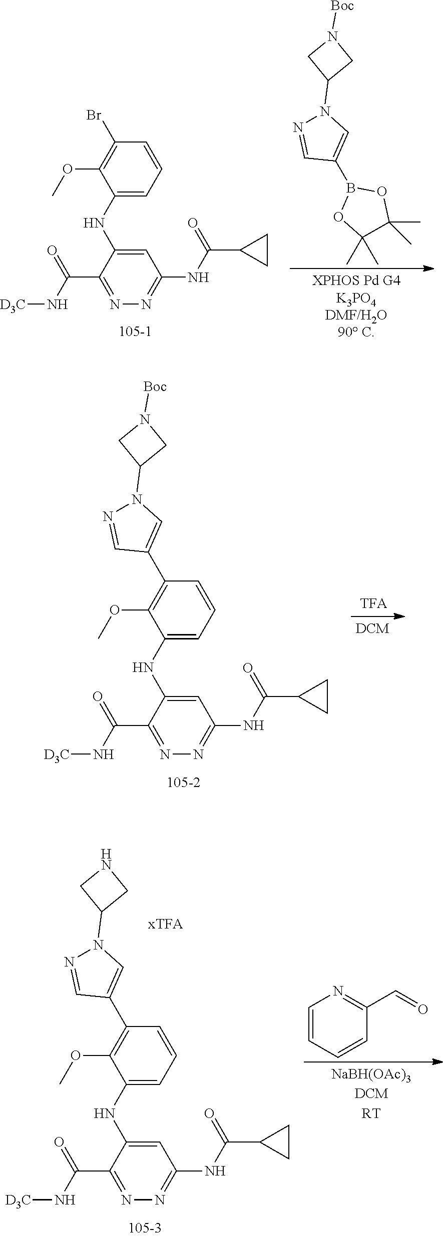 [SMILES: [2H]C([2H])([2H])NC(=O)c1nnc(NC(=O)C2CC2)cc1Nc1cccc(Br)c1OC ; CC(C)(C)OC(=O)N1CC(n2cc(B3OC(C)(C)C(C)(C)O3)cn2)C1 ; [2H]C([2H])([2H])NC(=O)c1nnc(NC(=O)C2CC2)cc1Nc1cccc(-c2cnn(C3CN(C(=O)OC(C)(C)C)C3)c2)c1OC ; [2H]C([2H])([2H])NC(=O)c1nnc(NC(=O)C2CC2)cc1Nc1cccc(-c2cnn(C3CNC3)c2)c1OC ; O=Cc1ccccn1]

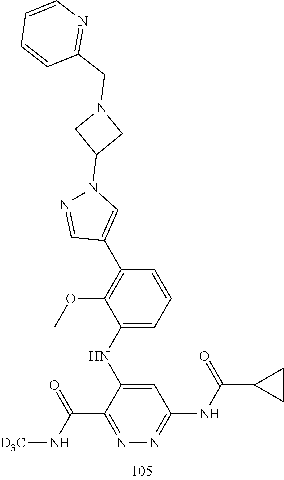 [SMILES: [2H]C([2H])([2H])NC(=O)c1nnc(NC(=O)C2CC2)cc1Nc1cccc(-c2cnn(C3CN(Cc4ccccn4)C3)c2)c1OC]

Compound 105-1 was prepared in analogous fashion to Intermediate H except that intermediate B-1-d3 was used.

Step 1: In a sealed tube degassed with nitrogen, a mixture of compound 105-1 (115 mg, 1.0 equiv, 272 μmol), tert-butyl 3-(4-(4,4,5,5-tetramethyl-1,3,2-dioxaborolan-2-yl)-1H-pyrazol-1-yl) azetidine-1-carboxylate (180 mg, 1.9 equiv, 516 μmol), (s)-(dicyclohexyl(2′,4′,6′-triisopropyl-[1,1′-biphenyl]-2-yl)-15-phosphaneyl) (2′-(methylamino)-[1,1′-biphenyl]-2-yl) palladium (III) methanesulfonate (XPhos Pd G4, 35.1 mg, 0.15 equiv, 40.8 μmol), and potassium phosphate (161 mg, 2.8 equiv, 761 μmol) in DMF (1 mL) and Water (0.3 mL) and was stirred at 90° C. for 90 min. The reaction mixture was cooled to RT and treated with water (500 mL). The resulting gray crude solid that formed was isolated by filtration, then dried overnight before purification by silica gel chromatography with a (gradient: DCM/10:90:0.5 MeOH/DCM/NHOH) to provide compound 105-2 (133.6 mg, 0.23 mmol, 85% yield) as a yellow, foamy solid.

LC/MS m/z (ES+): 566.5 [M+H]+

Step 2: A solution of compound 105-2 (133.6 mg, 98% Wt, 1.0 equiv, 231.5 μmol) in DCM (4 mL) and trifluoroacetic acid (791.7 mg, 528.2 μL, 30 Eq, 6.944 mmol) was stirred at RT. for 1 hour. The ensuing green solution was concentrated down and dried to yield 293 mg of crude compound 105-3 (TFA salt) as a thick oil, assumed to be quantitative: (187 mg, 231.5 μmol). LC-MS (ES+) m/z: 466.3 [M+H]+.

Step 3: A mixture of compound 105-3 (TFA salt) (293 mg, 64% Wt, 1.0 equiv, 232 μmol), picolinaldehyde (30.3 mg, 1.20 equiv, 283 μmol), and triethylamine (119 mg, 164 μL, 5.0 equiv, 1.18 mmol) in DCM (3 mL) was stirred 20 minutes at RT before slow addition of sodium triacetoxyborohydride (100 mg, 2.00 equiv, 472 μmol) over 20 minutes until the reaction was determined to be complete by LC/MS analysis. The reaction was diluted with DCM (10 mL) and treated with saturated aqueous NaHCO₃ (5 mL). The layers were separated, and the organic layer was dried over sodium sulfate, filtered, concentrated down, and dried.

The crude was purified by silica gel chromatography (gradient: DCM/10:90:0.5 MeOH/DCM/NH₄OH) to provide compound 105 (99.7 mg, 0.18 mmol, 76% yield) as a white solid.

LC/MS (ESI+) m/z: 557.4 [M+H]+

¹H NMR (500 MHz, DMSO) δ 11.29 (s, 1H), 10.96 (s, 1H), 9.12 (s, 1H), 8.49 (ddd, J=4.7, 1.9, 0.9 Hz, 1H), 8.36 (d, J=0.8 Hz, 1H), 8.13 (s, 1H), 8.01 (d, J=0.7 Hz, 1H), 7.76 (td, J=7.7, 1.8 Hz, 1H), 7.47 (dd, J=7.8, 1.6 Hz, 1H), 7.40 (dt, J=7.8, 1.2 Hz, 1H), 7.29 (dd, J=7.9, 1.5 Hz, 1H), 7.25 (ddd, J=7.5, 4.8, 1.2 Hz, 1H), 7.19 (t, J=7.9 Hz, 1H), 5.11 (p, J=6.9 Hz, 1H), 3.87-3.76 (m, 4H), 3.62-3.55 (m, 5H), 2.07 (tt, J=7.1, 5.3 Hz, 1H), 0.84-0.77 (m, 4H).

### Example 112

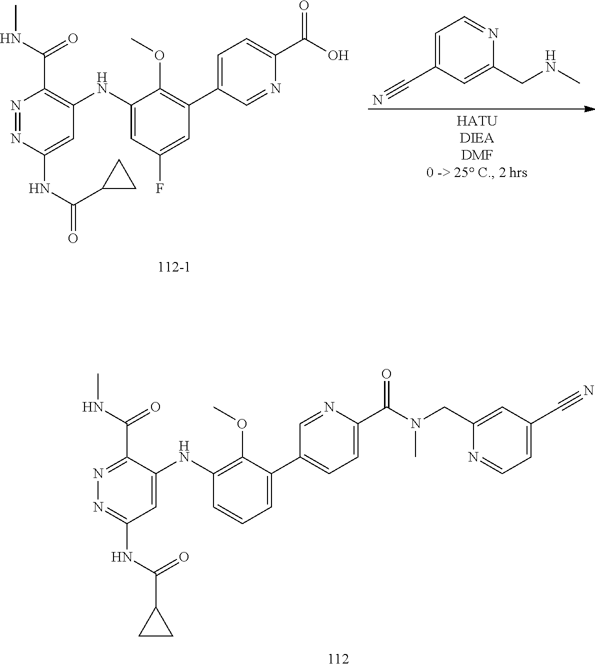 [SMILES: CNC(=O)c1nnc(NC(=O)C2CC2)cc1Nc1cc(F)cc(-c2ccc(C(=O)O)nc2)c1OC ; CNCc1cc(C#N)ccn1 ; CNC(=O)c1nnc(NC(=O)C2CC2)cc1Nc1cccc(-c2ccc(C(=O)N(C)Cc3cc(C#N)ccn3)nc2)c1OC]

Compound 112-1 was prepared in analogous fashion to Intermediate M except that Intermediate J was used in place of Intermediate H. Intermediate N was then coupled to compound 112-1 to provide compound 112 using analogous procedures to those in Example 18.

LCMS m/z (ES+): 610.2 [M+H]+

¹H NMR (400 MHZ, DMSO-d6) 11.42 (d, J=5.1 Hz, 1H), 11.17 (d, J=11.6 Hz, 1H), 9.21 (q, J=4.8 Hz, 1H), 8.89-8.81 (m, 1H), 8.80-8.65 (m, 1H), 8.27 (d, J=11.7 Hz, 1H), 8.15 (ddd, J=34.5, 8.1, 2.3 Hz, 1H), 7.88-7.66 (m, 3H), 7.44 (ddd, J=12.6, 9.7, 3.0 Hz, 1H), 7.21 (ddd, J=20.3, 9.0, 3.0 Hz, 1H), 4.89 (d, J=5.3 Hz, 2H), 3.40 (s, 3H), 3.09 (d, J=30.4 Hz, 3H), 2.86 (dd, J=4.9, 2.8 Hz, 3H), 2.10 (ddd, J=10.4, 5.6, 3.0 Hz, 1H), 0.90-0.81 (m, 4H).

TABLE 11
The following compounds were prepared in analogous fashion to compound
112 in Example 112.

| | | LCMS m/z | |
|---|---|---|---|
| Compound | Structure | (ES+) | ¹H NMR |
| 97 | 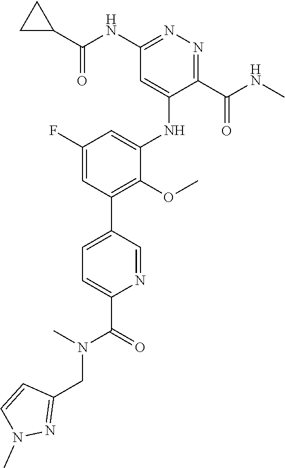 [SMILES: CNC(=O)c1nnc(NC(=O)C2CC2)cc1Nc1cc(F)cc(-c2ccc(C(=O)N(C)Cc3ccn(C)n3)nc2)c1OC] | 588.4 [M + H]+ | ¹H NMR (400 MHz, DMSO-d6) 11.43 (s, 1H), 11.17 (d, J = 4.5 Hz, 1H), 9.23 (d, J = 5.2 Hz, 1H), 8.80 (t, J = 3.0 Hz, 1H), 8.28 (d, J = 3.1 Hz, 1H), 8.16 (ddd, J = 7.8, 4.3, 2.2 Hz, 1H), 7.70 (d, J = 8.1 Hz, 1H), 7.63 (dd, J = 21.2, 2.1 Hz, 1H), 7.45 (dt, J = 9.8, 2.8 Hz, 1H), 7.23 (ddd, J = 8.7, 5.5, 3.0 Hz, 1H), 6.16 (dd, J = 31.5, 2.2 Hz, 1H), 4.58 (d, J = 35.1 Hz, 2H), 3.80 (d, J = 20.6 Hz, 3H), 3.38 (d, J = 4.0 Hz, 3H), 2.96 (d, J = 6.0 Hz, 3H), 2.86 (d, J = 4.8 Hz, 3H), 2.11 (dt, J = 12.8, 6.4 Hz, 1H), 0.86 (d, J = 6.2 Hz, 4H). |

### Example 118

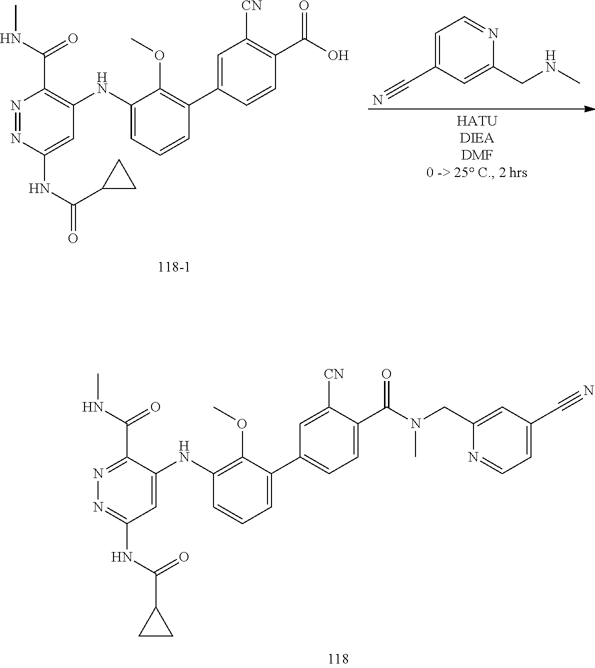 [SMILES: CNC(=O)c1nnc(NC(=O)C2CC2)cc1Nc1cccc(-c2ccc(C(=O)O)c(C#N)c2)c1OC ; CNCc1cc(C#N)ccn1 ; CNC(=O)c1nnc(NC(=O)C2CC2)cc1Nc1cccc(-c2ccc(C(=O)N(C)Cc3cc(C#N)ccn3)c(C#N)c2)c1OC]

Compound 118-1 was prepared in analogous fashion to the corresponding acid in Example 11 except that methyl 2-cyano-4-(4,4,5,5-tetramethyl-1,3,2-dioxaborolan-2-yl)benzoate was used in place of methyl 4-(4,4,5,5-tetramethyl-1,3,2-dioxaborolan-2-yl)benzoate. Intermediate N was then coupled to compound 118-1 to provide compound 118 using analogous procedures to those in Example 18.

LC/MS m/z (ES+): 616.4 [M+H]+

TABLE 12
Compounds in Table 12 were prepared according to the analogous procedures in
Example 118:

| | | LCMS m/z | |
|---|---|---|---|
| Compound | Structure | (ES+) | ¹H NMR |
| 119 | 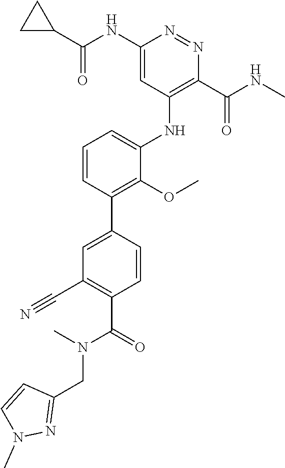 [SMILES: CNC(=O)c1nnc(NC(=O)C2CC2)cc1Nc1cccc(-c2ccc(C(=O)N(C)Cc3ccn(C)n3)c(C#N)c2)c1OC] | 594.5 [M + H]+ | |

### Example 121

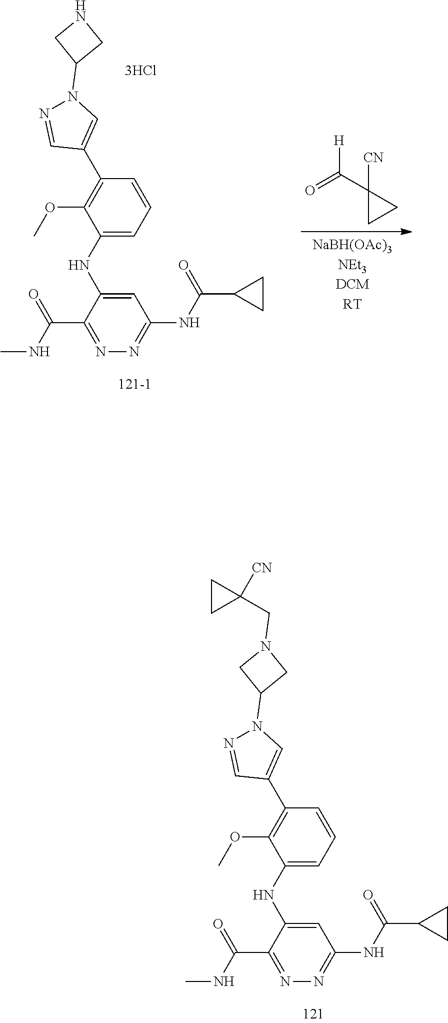 [SMILES: CNC(=O)c1nnc(NC(=O)C2CC2)cc1Nc1cccc(-c2cnn(C3CNC3)c2)c1OC ; N#CC1(C=O)CC1 ; CNC(=O)c1nnc(NC(=O)C2CC2)cc1Nc1cccc(-c2cnn(C3CN(CC4(C#N)CC4)C3)c2)c1OC]

A mixture of compound 121-1, 3HCl (80 mg, 92% Wt, 1.0 equiv, 0.13 mmol), triethylamine (65 mg, 89 μL, 5.0 equiv, 0.64 mmol), and 1-formylcyclopropane-1-carbonitrile (15 mg, 1.2 equiv, 0.15 mmol) in DCM (2 mL) was stirred 15 minutes at RT followed by the slow addition over 30 minutes of sodium triacetoxyborohydride (55 mg, 2.0 equiv, 0.26 mmol). About 30 minutes after final addition of reducing agent, the reaction was treated with DCM (5 mL) and saturated aqueous NaHCO₃ (5 mL). The layers were separated. The aqueous layer was re-extracted with fresh DCM (5 mL). The combined organic layers were dried over sodium sulfate, filtered, concentrated down, and purified by silica gel chromatography (gradient: DCM/10:90:0.5 MeOH/DCM/NH₄OH) to provide compound 121 (46.2 mg, 84 μmol, 66% yield) as a clear film lyophilized with tert-butanol to provide a white, amorphous solid.

LC/MS (ESI+): m/z=542.4 [M+H]+

¹H NMR (500 MHz, DMSO) δ 11.29 (s, 1H), 10.96 (s, 1H), 9.14 (q, J=4.8 Hz, 1H), 8.34 (d, J=0.8 Hz, 1H), 8.13 (s, 1H), 8.00 (s, 1H), 7.45 (dd, J=7.8, 1.5 Hz, 1H), 7.29 (dd, J=7.9, 1.5 Hz, 1H), 7.19 (t, J=7.9 Hz, 1H), 5.12 (p, J=7.1 Hz, 1H), 3.87-3.78 (m, 2H), 3.58 (s, 3H), 3.51 (td, J=6.8, 1.7 Hz, 2H), 2.85 (d, J=4.8 Hz, 3H), 2.61 (s, 2H), 2.07 (tt, J=7.2, 5.2 Hz, 1H), 1.17-1.12 (m, 2H), 0.97-0.91 (m, 2H), 0.83-0.78 (m, 4H).

### Example 127

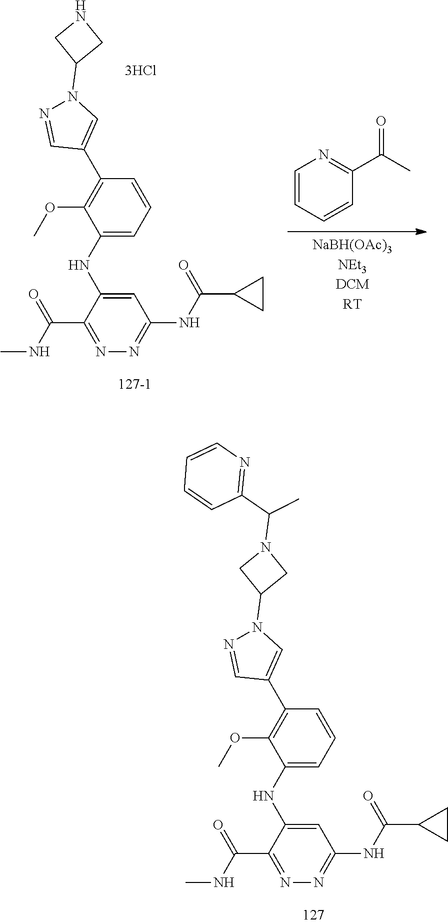

A mixture of compound 127-1, 3HCl (160 mg, 1.0 equiv, 257 μmol), triethylamine (130 mg, 179 μL, 5.0 equiv, 1.29 mmol), and 1-(pyridin-2-yl) ethan-1-one (37.4 mg, 1.2 equiv, 309 μmol) in DCM (4 mL) was stirred 15 minutes at RT followed by the slow addition over 30 minutes of sodium triacetoxyborohydride (109 mg, 2.0 equiv, 515 μmol). The reaction was allowed to stir for 20 minutes after addition of reducing agent, after which the reaction mixture was treated with DCM (5 mL) and saturated aqueous NaHCO₃ (5 mL). The layers were separated. The aqueous layer was re-extracted with fresh DCM (5 mL). The combined organic layers were dried over sodium sulfate, filtered, and concentrated down. The crude was purified by silica gel chromatography (gradient: DCM/10:90:0.5 MeOH/DCM/NH₄OH) to provide racemic compound 127 (99.1 mg, 0.17 mmol, 67% yield) as a white, foamy solid.

LC/MS (ESI+) m/z: 568.4 [M+H]+.

¹H NMR (400 MHz, DMSO) δ 11.30 (s, 1H), 10.96 (s, 1H), 9.15 (q, J=4.7 Hz, 1H), 8.49 (ddd, J=4.8, 1.8, 0.9 Hz, 1H), 8.35 (d, J=0.7 Hz, 1H), 8.13 (s, 1H), 8.00 (d, J=0.7 Hz, 1H), 7.76 (td, J=7.7, 1.8 Hz, 1H), 7.49-7.41 (m, 2H), 7.28 (dd, J=7.9, 1.6 Hz, 1H), 7.25 (ddd, J=7.5, 4.8, 1.2 Hz, 1H), 7.19 (t, J=7.9 Hz, 1H), 5.05 (p, J=7.1 Hz, 1H), 3.80 (t, J=7.1 Hz, 1H), 3.63 (q, J=6.6 Hz, 1H), 3.59-3.49 (m, 5H), 3.40 (t, J=6.8 Hz, 1H), 2.85 (d, J=4.8 Hz, 3H), 2.12-2.02 (m, 1H), 1.20 (d, J=6.6 Hz, 3H), 0.84-0.75 (m, 4H).

The enantiomers of compound 127 were separated to provide compounds 162 and 163 (absolute stereochemistry arbitrarily assigned):

### Example 162

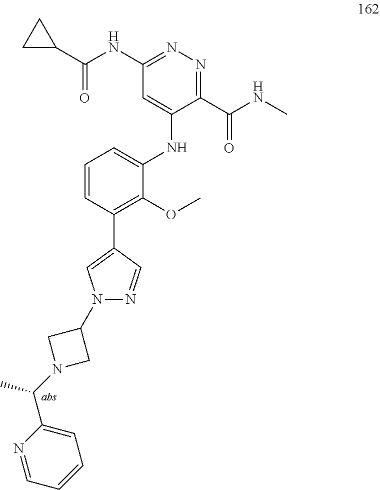 [SMILES: CNC(=O)c1nnc(NC(=O)C2CC2)cc1Nc1cccc(-c2cnn(C3CN([C@@H](C)c4ccccn4)C3)c2)c1OC]

LC/MS (ESI+) m/z: 568.4 [M+H]+.

### Example 163

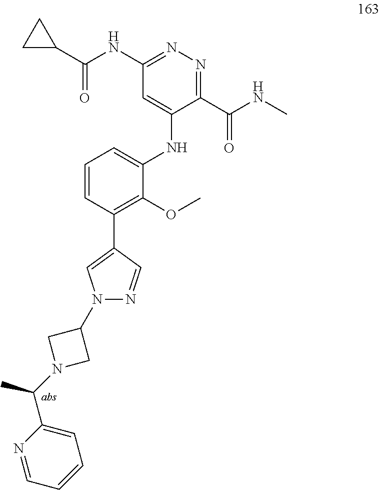 [SMILES: CNC(=O)c1nnc(NC(=O)C2CC2)cc1Nc1cccc(-c2cnn(C3CN([C@H](C)c4ccccn4)C3)c2)c1OC]

LC/MS (ESI+) m/z: 568.4 [M+H]+.

### Example 131

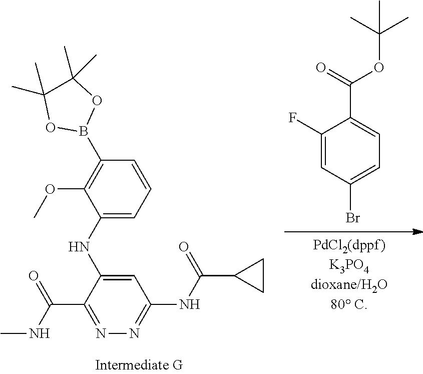 [SMILES: CNC(=O)c1nnc(NC(=O)C2CC2)cc1Nc1cccc(B2OC(C)(C)C(C)(C)O2)c1OC ; CC(C)(C)OC(=O)c1ccc(Br)cc1F]

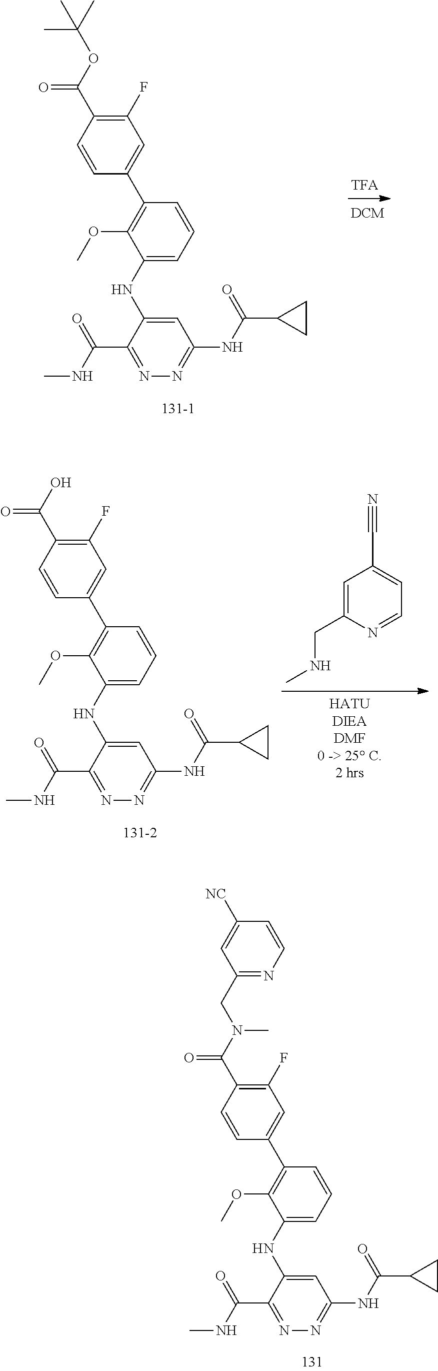 [SMILES: CNC(=O)c1nnc(NC(=O)C2CC2)cc1Nc1cccc(-c2ccc(C(=O)OC(C)(C)C)c(F)c2)c1OC ; CNC(=O)c1nnc(NC(=O)C2CC2)cc1Nc1cccc(-c2ccc(C(=O)O)c(F)c2)c1OC ; CNCc1cc(C#N)ccn1 ; CNC(=O)c1nnc(NC(=O)C2CC2)cc1Nc1cccc(-c2ccc(C(=O)N(C)Cc3cc(C#N)ccn3)c(F)c2)c1OC]

Step 1: A screw cap vial was charged with Intermediate G (200 mg, 1.0 equiv, 428 μmol), tert-butyl 4-bromo-2-fluorobenzoate (235 mg, 2.0 equiv, 856 μmol), 1,1′-bis(diphenylphosphino) ferrocene-palladium (II) dichloride (31.3 mg, 0.1 equiv, 42.8 μmol) and potassium phosphate (182 mg, 2.0 equiv, 856 μmol). The vial was degassed with nitrogen and filled with DMF (5.0 mL) and water (1.7 mL) after which it was heated at 80° C. After 16 h, the reaction was determined to be complete by LC/MS analysis. The reaction was cooled to RT, diluted with EtOAc and extracted with sat. NH₄Cl and brine. The organic layer was dried over Na₂SO₄, concentrated and purified by flash silica gel chromatography (Combiflash, gradient ethyl acetate/heptanes). The product containing fractions were concentrated, yielding compound 131-1 as a tan solid.

LC/MS (ESI+): m/z=536.5 [M+H]+

Step 2: A vial was loaded with compound 131-1 (225 mg, 1.0 equiv, 420 μmol), after which TFA and DCM was added (5 mL, 1:1 v/v). The vial was stirred at RT for 30 min, after which there was determined to be no more SM by LC/MS analysis. The reaction was concentrated and used without further purification.

LC/MS (ESI+): m/z=480.3 [M+H]+

Step 3: Acid 131-2 was converted to corresponding amide 131 using the analogous coupling conditions from Example 18.

LC/MS (ESI+): m/z=609.4 [M+H]+.

TABLE 13
Compounds in Table 13 were prepared according to the analogous procedures in
Example 131:

| | | LCMS m/z | |
|---|---|---|---|
| Compound | Structure | (ES+) | ¹H NMR |
| 132 | 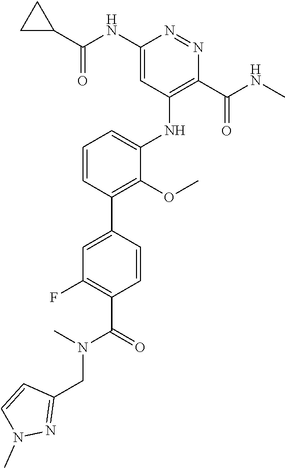 [SMILES: CNC(=O)c1nnc(NC(=O)C2CC2)cc1Nc1cccc(-c2ccc(C(=O)N(C)Cc3ccn(C)n3)c(F)c2)c1OC] | 587.5 [M + H]+ | |

### Example 137

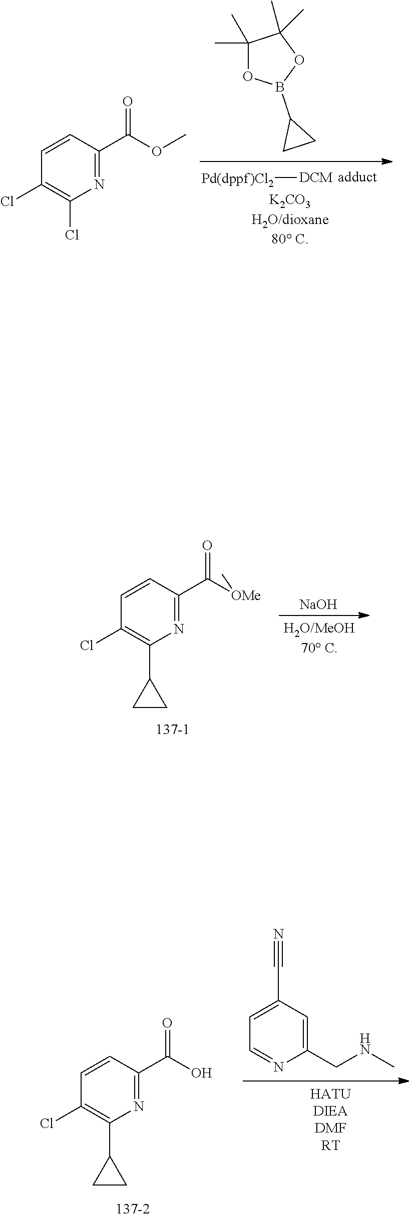

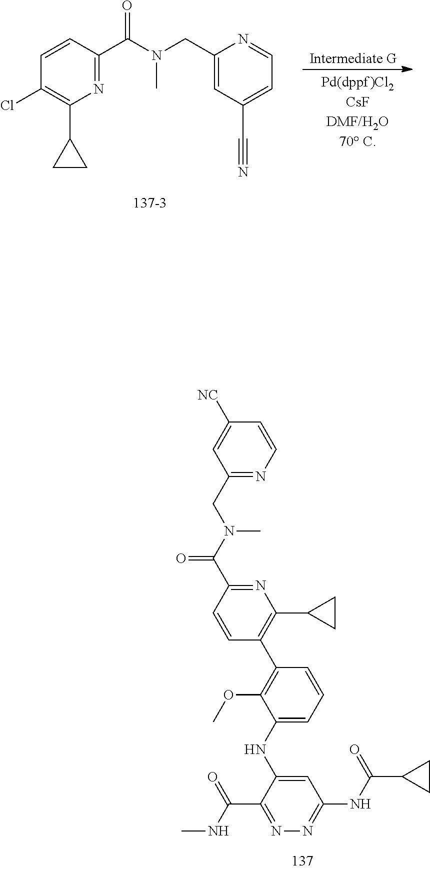 [SMILES: CN(Cc1cc(C#N)ccn1)C(=O)c1ccc(Cl)c(C2CC2)n1 ; CNC(=O)c1nnc(NC(=O)C2CC2)cc1Nc1cccc(-c2ccc(C(=O)N(C)Cc3cc(C#N)ccn3)nc2C2CC2)c1OC]

Step 1: A round bottomed flask was charged with methyl 5,6-dichloropicolinate (2.0 g, 1.0 equiv, 0.01 mol), 2-cyclopropyl-4,4,5,5-tetramethyl-1,3,2-dioxaborolane (1.2 equiv, 0.01 mol), K₂CO₃ (4.0 g, 3.0 equiv, 0.03 mol), PdCl₂(dppf)-CH₂Cl₂ adduct (0.2 equiv, 2 mmol). 1,4-dioxane (8 mL) and water (2 mL) were added, and the solution was stirred at 80° C. for 12 hour under N₂. The reaction was quenched with water and extracted with ethyl acetate. The organic layer was washed with brine, dried over Na₂SO₄ and evaporated. The crude product was purified by prepTLC (ethyl acetate/petroleum ether, 1/1 v:v) to provide compound 137-1 (900 mg, 4.25 mmol, 40%) as an off-white solid.

LC/MS (ES+) m/z: 212.10 [M+H]+

Step 2: A round bottomed flask was charged with compound 137-1 (700 mg, 1.0 equiv, 3.31 mmol) in MeOH (5 mL), followed by NaOH (0.26 g, 2.0 equiv, 6.61 mmol) in water (5 mL), and the solution was stirred at 70° C. for 12 hour. The reaction was then quenched with 1M HCl and extracted with ethyl acetate. The organic layer was dried over Na₂SO₄, evaporated and purified using C18 flash chromatography (gradient MeCN/water) to provide compound 137-2 (200 mg, 1.01 mmol, 31% yield) as a yellow oil.

LC/MS (ES+) m/z: 197.90 [M+H]+

Step 3: A round bottomed flask was charged with compound 137-2, 2-((methylamino)methyl) isonicotinonitrile (179 mg, 1.2 equiv, 1.21 mmol), DIEA (392 mg, 529 μL, 3.0 equiv, 3.04 mmol), and HATU (577 mg, 1.5 equiv, 1.52 mmol). DMF (3 mL) was added, and the solution was stirred at 25° C. for 2 hour. The resulted solution was purified using C18 flash chromatography (gradient water/MeCN) to provide compound 137-3 (230 mg, 704 μmol, 70% yield) as an off-white solid.

LC/MS (ES+) m/z: 327.0 [M+H]+

Step 4: A round bottomed flask was charged with compound 137-3 (100 mg, 1.0 equiv, 306 μmol), Intermediate G (143 mg, 1.0 equiv, 306 μmol), CsF (93.0 mg, 2.0 equiv, 612 μmol) 1,1′-bis(di-t-butylphosphino) ferrocene palladium dichloride (39.9 mg, 0.2 equiv, 61.2 μmol). DMF (4 mL) and water (0.8 mL) were then added, and the solution was stirred at 80° C. for 2 hour under N₂. The resulted solution was purified using PREP-HPLC. Lyophilization of product-containing fractions provided compound 137 (64.5 mg, 102 μmol, 33% yield) as an off-white amorphous solid.

LC/MS (ES+) m/z 632.25 [M+H]+

¹H NMR (400 MHZ, DMSO-d6) 11.33 (d, J=6.4 Hz, 1H), 10.92 (d, J=11.1 Hz, 1H), 9.16 (t, J=5.0 Hz, 1H), 8.87-8.78 (m, 1H), 8.13-7.67 (m, 4H), 7.59-7.44 (m, 2H), 7.38-7.07 (m, 2H), 4.83 (dd, J=17.2, 5.7 Hz, 2H), 3.38 (s, 1H), 3.31 (s, 2H), 3.08 (d, J=24.7 Hz, 3H), 2.85 (t, J=4.5 Hz, 3H), 2.09 (d, J=6.2 Hz, 1H), 1.96-1.68 (m, 1H), 0.99-0.74 (m, 6H), 0.61 (dd, J=8.1, 3.6 Hz, 1H), 0.33 (d, J=44.1 Hz, 1H).

### Example 147

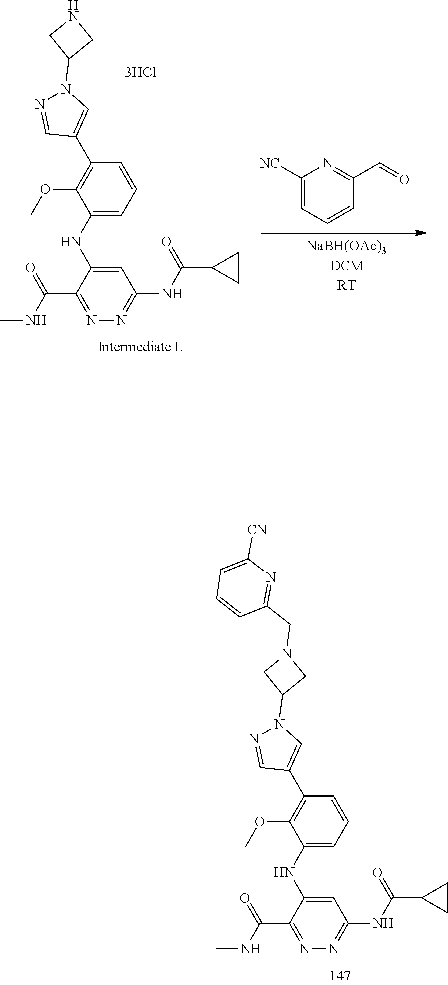 [SMILES: CNC(=O)c1nnc(NC(=O)C2CC2)cc1Nc1cccc(-c2cnn(C3CNC3)c2)c1OC ; N#Cc1cccc(C=O)n1 ; CNC(=O)c1nnc(NC(=O)C2CC2)cc1Nc1cccc(-c2cnn(C3CN(Cc4cccc(C#N)n4)C3)c2)c1OC]

A mixture of compound Intermediate L, 3HCl (80 mg, 1.0 equiv, 0.13 mmol) and 6-formylpicolinonitrile (20 mg, 1.2 equiv, 0.15 mmol) in DCM (2 mL) was stirred 15 minutes at RT followed by the slow addition over 30 minutes of sodium triacetoxyborohydride (55 mg, 2.0 equiv, 0.26 mmol). The reaction was then stirred at RT for 2 h after which there was no more starting material determined by LC/MS analysis. The reaction was treated with DCM (5 mL) and saturated aqueous NaHCO₃ (5 mL) after which the layers were separated and the aqueous layer was re-extracted with fresh DCM (5 mL). The combined organic layers were dried over sodium sulfate, filtered, concentrated down, and purified by silica gel chromatography (gradient: DCM to 10:90:0.5 MeOH/DCM/NH₄OH) to provide compound 147 (42.8 mg, 70.5 μmol, 55% yield) as a white solid.

LCMS m/z (ES+): 579.4 [M+H]+

¹H NMR (500 MHZ, DMSO) δ 11.29 (s, 1H), 10.96 (s, 1H), 9.14 (q, J=5.0 Hz, 1H), 8.37 (d, J=0.8 Hz, 1H), 8.13 (s, 1H), 8.04 (t, J=7.8 Hz, 1H), 8.02 (s, 1H), 7.92 (dd, J=7.6, 1.1 Hz, 1H), 7.75 (dd, J=8.0, 1.1 Hz, 1H), 7.47 (dd, J=7.9, 1.6 Hz, 1H), 7.29 (dd, J=7.9, 1.6 Hz, 1H), 7.19 (t, J=7.9 Hz, 1H), 5.13 (p, J=6.8 Hz, 1H), 3.90 (s, 2H), 3.82 (t, J=7.3 Hz, 2H), 3.66-3.54 (m, 5H), 2.85 (d, J=4.8 Hz, 3H), 2.10-2.02 (m, 1H), 0.85-0.73 (m, 4H).

TABLE 14
The following compounds were prepared in analogous fashion to compound
147 in Example 147:

| | | LCMS | |
|---|---|---|---|
| Compound | Structure | m/z (ES+) | ¹H NMR |
| 39 | 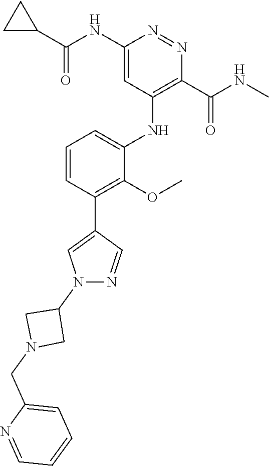 [SMILES: CNC(=O)c1nnc(NC(=O)C2CC2)cc1Nc1cccc(-c2cnn(C3CN(Cc4ccccn4)C3)c2)c1OC] | 554.5 [M + H]+ | ¹H NMR (500 MHz, DMSO) δ 11.30 (s, 1H), 10.98 (s, 1H), 9.16 (q, J = 4.8 Hz, 1H), 8.51 (ddd, J = 4.8, 1.8, 0.9 Hz, 1H), 8.38 (d, J = 0.7 Hz, 1H), 8.15 (s, 1H), 8.03 (d, J = 0.7 Hz, 1H), 7.78 (td, J = 7.7, 1.9 Hz, 1H), 7.48 (dd, J = 7.8, 1.5 Hz, 1H), 7.42 (dt, J = 7.8, 1.1 Hz, 1H), 7.30 (dd, J = 7.9, 1.5 Hz, 1H), 7.27 (ddd, J = 7.6, 4.8, 1.2 Hz, 1H), 7.21 (t, J = 7.9 Hz, 1H), 5.12 (p, J = 6.9 Hz, 1H), 3.85 (s, 2H), 3.82 (td, J = 7.1, 1.7 Hz, 2H), 3.63- 3.57 (m, 5H), 2.87 (d, J = 4.7 Hz, 3H), 2.09 (tt, J = 7.0, 5.4 Hz, 1H), 0.83 (dt, J = 7.6, 2.0 Hz, 4H). |
| 41 | 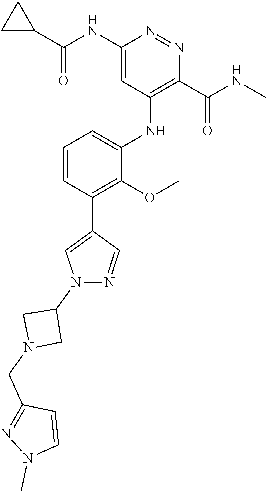 [SMILES: CNC(=O)c1nnc(NC(=O)C2CC2)cc1Nc1cccc(-c2cnn(C3CN(Cc4ccn(C)n4)C3)c2)c1OC] | 557.4 [M + H]+ | ¹H NMR (400 MHz, DMSO) δ 11.30 (s, 1H), 10.96 (s, 1H), 9.19-9.12 (m, 1H), 8.31 (d, J = 0.8 Hz, 1H), 8.14 (s, 1H), 8.00 (d, J = 0.7 Hz, 1H), 7.57 (d, J = 2.1 Hz, 1H), 7.46 (dd, J = 7.8, 1.6 Hz, 1H), 7.28 (dd, J = 7.9, 1.6 Hz, 1H), 7.19 (t, J = 7.9 Hz, 1H), 6.12 (d, J = 2.1 Hz, 1H), 5.03 (p, J = 7.0 Hz, 1H), 3.77 (s, 3H), 3.69 (td, J = 7.0, 1.7 Hz, 2H), 3.60 (s, 2H), 3.57 (s, 3H), 3.47 (td, J = 6.8, 1.6 Hz, 2H), 2.85 (d, J = 4.8 Hz, 3H), 2.11- 2.02 (m, 1H), 0.88-0.74 (m, 4H). |
| 44 | 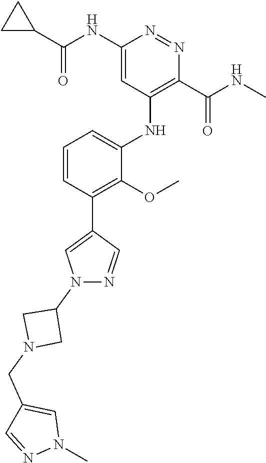 [SMILES: CNC(=O)c1nnc(NC(=O)C2CC2)cc1Nc1cccc(-c2cnn(C3CN(Cc4cnn(C)c4)C3)c2)c1OC] | 557.4 [M + H]+ | |
| 66 | 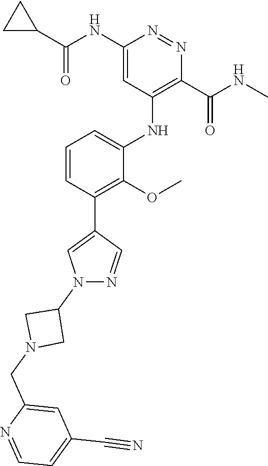 [SMILES: CNC(=O)c1nnc(NC(=O)C2CC2)cc1Nc1cccc(-c2cnn(C3CN(Cc4cc(C#N)ccn4)C3)c2)c1OC] | 579.4 [M + H]+ | ¹H NMR (400 MHz, DMSO) δ 11.30 (s, 1H), 10.97 (s, 1H), 9.19-9.12 (m, 1H), 8.76 (dd, J = 5.0, 0.9 Hz, 1H), 8.38 (d, J = 0.7 Hz, 1H), 8.14 (s, 1H), 8.02 (d, J = 0.7 Hz, 1H), 7.82-7.80 (m, 1H), 7.75 (dd, J = 5.0, 1.6 Hz, 1H), 7.47 (dd, J = 7.8, 1.6 Hz, 1H), 7.29 (dd, J = 7.9, 1.6 Hz, 1H), 7.19 (t, J = 7.9 Hz, 1H), 5.12 (p, J = 7.0 Hz, 1H), 3.91 (s, 2H), 3.84 (t, J = 7.7 Hz, 2H), 3.65-3.54 (m, 5H), 2.85 (d, J = 4.8 Hz, 3H), 2.07 (ddd, J = 12.5, 7.3, 5.3 Hz, 1H), 0.85- 0.75 (m, 4H). |
| 69 | 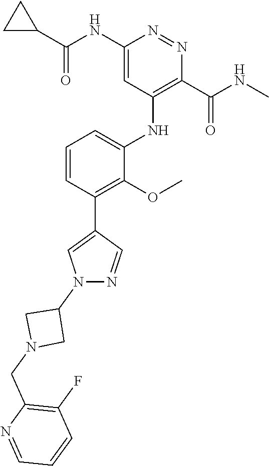 [SMILES: CNC(=O)c1nnc(NC(=O)C2CC2)cc1Nc1cccc(-c2cnn(C3CN(Cc4ncccc4F)C3)c2)c1OC] | 572.5 [M + H]+ | ¹H NMR (400 MHz, DMSO) δ 11.30 (s, 1H), 10.96 (s, 1H), 9.20-9.11 (m, 1H), 8.38 (dt, J = 4.7, 1.5 Hz, 1H), 8.32 (d, J = 0.8 Hz, 1H), 8.13 (s, 1H), 8.00 (d, J = 0.7 Hz, 1H), 7.70 (ddd, J = 9.9, 8.4, 1.3 Hz, 1H), 7.46 (dd, J = 7.8, 1.6 Hz, 1H), 7.41 (dt, J = 8.5, 4.4 Hz, 1H), 7.28 (dd, J = 8.0, 1.5 Hz, 1H), 7.18 (t, J = 7.9 Hz, 1H), 5.05 (p, J = 7.0 Hz, 1H), 3.86 (d, J = 2.5 Hz, 2H), 3.76 (t, J = 7.3 Hz, 2H), 3.63 (t, J = 7.3 Hz, 2H), 3.57 (s, 3H), 2.85 (d, J = 4.8 Hz, 3H), 2.07 (p, J = 7.0 Hz, 1H), 0.87-0.74 (m, 4H). |
| 70 | 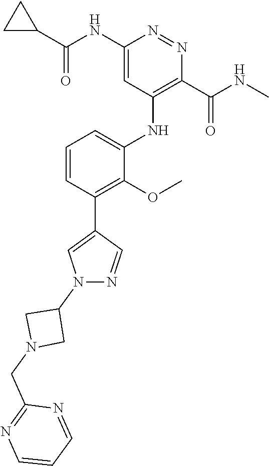 [SMILES: CNC(=O)c1nnc(NC(=O)C2CC2)cc1Nc1cccc(-c2cnn(C3CN(Cc4ncccn4)C3)c2)c1OC] | 555.3 [M + H]+ | ¹H NMR (400 MHz, DMSO) δ 11.30 (s, 1H), 10.96 (s, 1H), 9.15 (q, J = 4.7 Hz, 1H), 8.77 (d, J = 4.9 Hz, 2H), 8.35 (d, J = 0.7 Hz, 1H), 8.14 (s, 1H), 8.00 (d, J = 0.7 Hz, 1H), 7.46 (dd, J = 7.8, 1.6 Hz, 1H), 7.40 (t, J = 4.9 Hz, 1H), 7.28 (dd, J = 8.0, 1.6 Hz, 1H), 7.19 (t, J = 7.9 Hz, 1H), 5.09 (p, J = 6.9 Hz, 1H), 3.90 (s, 2H), 3.86 (dd, J = 8.3, 7.0 Hz, 2H), 3.66 (dd, J = 8.3, 6.7 Hz, 2H), 3.57 (s, 3H), 2.85 (d, J = 4.8 Hz, 3H), 2.07 (p, J = 6.6 Hz, 1H), 0.84-0.77 (m, 4H). |
| 77 | 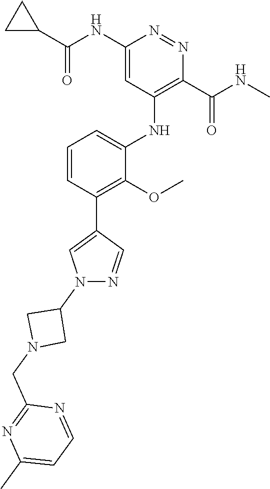 | 569.6 [M + H]+ | ¹H NMR (400 MHz, DMSO) δ 11.29 (s, 1H), 10.96 (s, 1H), 9.15 (q, J = 4.7 Hz, 1H), 8.59 (d, J = 5.1 Hz, 1H), 8.35 (d, J = 0.8 Hz, 1H), 8.14 (s, 1H), 8.00 (d, J = 0.7 Hz, 1H), 7.46 (dd, J = 7.8, 1.6 Hz, 1H), 7.28 (dd, J = 7.9, 1.6 Hz, 1H), 7.26 (d, J = 5.1 Hz, 1H), 7.19 (t, J = 7.9 Hz, 1H), 5.08 (p, J = 7.0 Hz, 1H), 3.89-3.79 (m, 4H), 3.64 (td, J = 6.7, 1.6 Hz, 2H), 3.57 (s, 3H), 2.85 (d, J = 4.8 Hz, 3H), 2.44 (s, 3H), 2.10-2.03 (m, 1H), 0.85- 0.76 (m, 4H). |
| 78 | 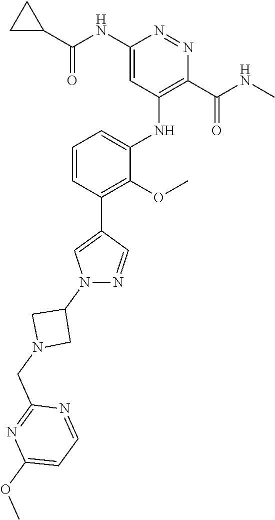 [SMILES: CNC(=O)c1nnc(NC(=O)C2CC2)cc1Nc1cccc(-c2cnn(C3CN(Cc4nccc(OC)n4)C3)c2)c1OC] | 585.5 [M + H]+ | ¹H NMR (400 MHz, DMSO) δ 11.29 (s, 1H), 10.96 (s, 1H), 9.15 (q, J = 4.7 Hz, 1H), 8.45 (d, J = 5.8 Hz, 1H), 8.36 (d, J = 0.8 Hz, 1H), 8.14 (s, 1H), 8.01 (d, J = 0.7 Hz, 1H), 7.46 (dd, J = 7.8, 1.6 Hz, 1H), 7.28 (dd, J = 8.0, 1.6 Hz, 1H), 7.19 (t, J = 7.9 Hz, 1H), 6.81 (d, J = 5.8 Hz, 1H), 5.11 (p, J = 7.0 Hz, 1H), 3.95-3.85 (m, 5H), 3.81 (s, 2H), 3.68 (dd, J = 8.0, 6.5 Hz, 2H), 3.57 (s, 3H), 2.85 (d, J = 4.7 Hz, 3H), 2.11-2.03 (m, 1H), 0.83-0.75 (m, 4H). |
| 88 | 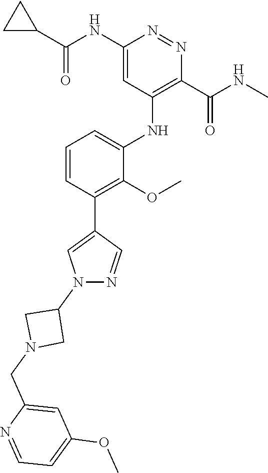 [SMILES: CNC(=O)c1nnc(NC(=O)C2CC2)cc1Nc1cccc(-c2cnn(C3CN(Cc4cc(OC)ccn4)C3)c2)c1OC] | 584.6 [M + H]+ | ¹H NMR (500 MHz, DMSO) δ 11.29 (s, 1H), 10.96 (s, 1H), 9.14 (q, J = 4.8 Hz, 1H), 8.36 (d, J = 0.8 Hz, 1H), 8.30 (d, J = 5.7 Hz, 1H), 8.14 (s, 1H), 8.01 (s, 1H), 7.46 (dd, J = 7.8, 1.5 Hz, 1H), 7.29 (dd, J = 7.9, 1.5 Hz, 1H), 7.19 (t, J = 7.9 Hz, 1H), 6.95 (d, J = 2.6 Hz, 1H), 6.84 (dd, J = 5.7, 2.6 Hz, 1H), 5.11 (p, J = 7.0 Hz, 1H), 3.86-3.75 (m, 7H), 3.61-3.53 (m, 5H), 2.86 (d, J = 4.9 Hz, 3H), 2.10-2.02 (m, 1H), 0.83-0.77 (m, 4H). |
| 89 | 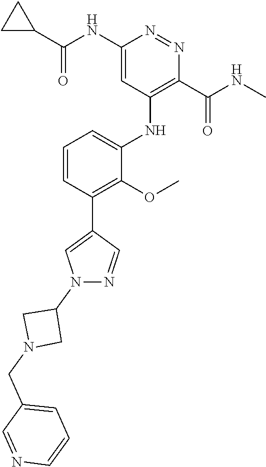 [SMILES: CNC(=O)c1nnc(NC(=O)C2CC2)cc1Nc1cccc(-c2cnn(C3CN(Cc4cccnc4)C3)c2)c1OC] | 554.4 [M + H]+ | ¹H NMR (500 MHz, DMSO) δ 11.29 (s, 1H), 10.96 (s, 1H), 9.14 (q, J = 4.8 Hz, 1H), 8.54-8.51 (m, 1H), 8.46 (dd, J = 4.8, 1.7 Hz, 1H), 8.35 (d, J = 0.8 Hz, 1H), 8.13 (s, 1H), 8.01 (s, 1H), 7.73 (dt, J = 7.8, 2.0 Hz, 1H), 7.46 (dd, J = 7.8, 1.5 Hz, 1H), 7.35 (ddd, J = 7.8, 4.7, 0.9 Hz, 1H), 7.29 (dd, J = 7.9, 1.6 Hz, 1H), 7.19 (t, J = 7.9 Hz, 1H), 5.09 (p, J = 6.9 Hz, 1H), 3.79-3.66 (m, 4H), 3.58 (s, 3H), 3.52 (t, J = 7.0 Hz, 2H), 2.85 (d, J = 4.9 Hz, 3H), 2.06 (td, J = 7.1, 3.7 Hz, 1H), 0.83-0.77 (m, 4H). |
| 90 | 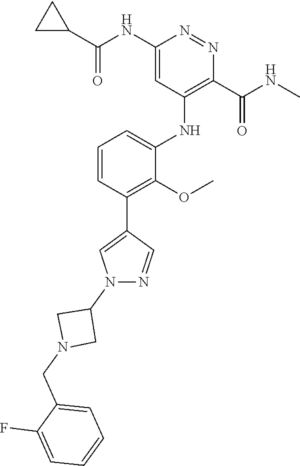 [SMILES: CNC(=O)c1nnc(NC(=O)C2CC2)cc1Nc1cccc(-c2cnn(C3CN(Cc4ccccc4F)C3)c2)c1OC] | 571.4 [M + H]+ | ¹H NMR (500 MHz, DMSO) δ 11.29 (s, 1H), 10.96 (s, 1H), 9.14 (q, J = 4.8 Hz, 1H), 8.34 (d, J = 0.8 Hz, 1H), 8.13 (s, 1H), 8.01 (s, 1H), 7.48-7.41 (m, 2H), 7.34-7.27 (m, 2H), 7.21-7.14 (m, 3H), 5.12-5.02 (m, 1H), 3.80- 3.68 (m, 4H), 3.60-3.50 (m, 5H), 2.85 (d, J = 4.8 Hz, 3H), 2.11-2.01 (m, 1H), 0.83-0.77 (m, 4H). |
| 91 | 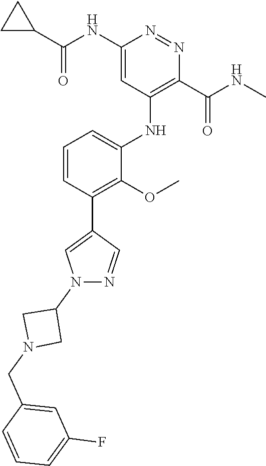 [SMILES: CNC(=O)c1nnc(NC(=O)C2CC2)cc1Nc1cccc(-c2cnn(C3CN(Cc4cccc(F)c4)C3)c2)c1OC] | 571.4 [M + H]+ | ¹H NMR (500 MHz, DMSO) δ 11.31 (s, 1H), 10.98 (s, 1H), 9.16 (q, J = 4.7 Hz, 1H), 8.37 (d, J = 0.7 Hz, 1H), 8.15 (s, 1H), 8.03 (s, 1H), 7.48 (dd, J = 7.9, 1.6 Hz, 1H), 7.38 (td, J = 7.9, 6.1 Hz, 1H), 7.31 (dd, J = 7.9, 1.6 Hz, 1H), 7.24-7.14 (m, 3H), 7.08 (td, J = 8.2, 2.3 Hz, 1H), 5.11 (p, J = 7.0 Hz, 1H), 3.81-3.70 (m, 4H), 3.60 (s, 3H), 3.52 (t, J = 7.1 Hz, 2H), 2.87 (d, J = 4.8 Hz, 3H), 2.12-2.05 (m, 1H), 0.85-0.80 (m, 4H). |
| 104 | 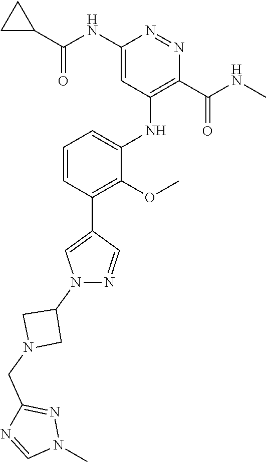 [SMILES: CNC(=O)c1nnc(NC(=O)C2CC2)cc1Nc1cccc(-c2cnn(C3CN(Cc4ncn(C)n4)C3)c2)c1OC] | 558.5 [M + H]+ | ¹H NMR (500 MHz, DMSO) δ 11.29 (s, 1H), 10.96 (s, 1H), 9.14 (q, J = 4.7 Hz, 1H), 8.36 (s, 1H), 8.32 (d, J = 0.8 Hz, 1H), 8.13 (s, 1H), 7.99 (d, J = 0.7 Hz, 1H), 7.45 (dd, J = 7.9, 1.6 Hz, 1H), 7.28 (dd, J = 7.9, 1.5 Hz, 1H), 7.19 (t, J = 7.9 Hz, 1H), 5.04 (p, J = 6.9 Hz, 1H), 3.82 (s, 3H), 3.76 (t, J = 7.4 Hz, 2H), 3.68 (s, 2H), 3.61-3.56 (m, 5H), 2.86 (d, J = 4.8 Hz, 3H), 2.12-2.03 (m, 1H), 0.82-0.78 (m, 4H). |
| 106 | 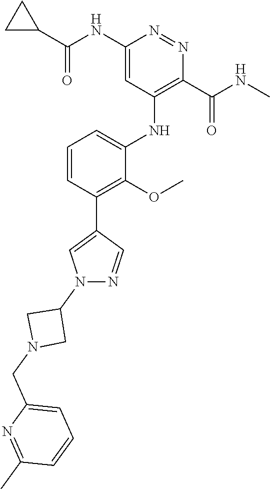 | 568.5 [M + H]+ | ¹H NMR (500 MHz, DMSO) δ 11.29 (s, 1H), 10.96 (s, 1H), 9.14 (q, J = 4.7 Hz, 1H), 8.36 (d, J = 0.8 Hz, 1H), 8.13 (s, 1H), 8.01 (d, J = 0.7 Hz, 1H), 7.63 (t, J = 7.7 Hz, 1H), 7.47 (dd, J = 7.8, 1.5 Hz, 1H), 7.29 (dd, J = 7.9, 1.5 Hz, 1H), 7.21-7.16 (m, 2H), 7.10 (d, J = 7.6 Hz, 1H), 5.10 (p, J = 6.9 Hz, 1H), 3.82-3.75 (m, 4H), 3.61-3.52 (m, 5H), 2.85 (d, J = 4.9 Hz, 3H), 2.43 (s, 3H), 2.10-2.03 (m, 1H), 0.83-0.77 (m, 4H). |
| 107 | 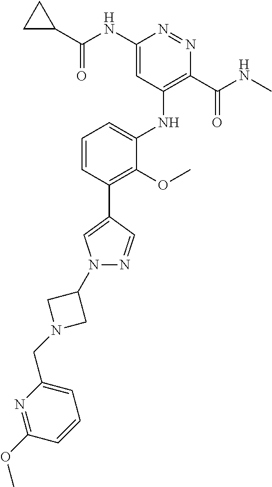 [SMILES: CNC(=O)c1nnc(NC(=O)C2CC2)cc1Nc1cccc(-c2cnn(C3CN(Cc4cccc(OC)n4)C3)c2)c1OC] | 584.5 [M + H]+ | ¹H NMR (500 MHz, DMSO) δ 11.29 (s, 1H), 10.96 (s, 1H), 9.17-9.12 (m, 1H), 8.37 (s, 1H), 8.13 (s, 1H), 8.02 (s, 1H), 7.66 (t, J = 7.8 Hz, 1H), 7.47 (dd, J = 7.8, 1.6 Hz, 1H), 7.29 (dd, J = 7.9, 1.5 Hz, 1H), 7.19 (t, J = 7.9 Hz, 1H), 6.96 (d, J = 7.3 Hz, 1H), 6.68 (d, J = 8.3 Hz, 1H), 5.21-5.05 (m, 1H), 3.94-3.71 (m, 7H), 3.66- 3.53 (m, 5H), 2.85 (d, J = 4.9 Hz, 3H), 2.10-2.03 (m, 1H), 0.83-0.76 (m, 4H). |
| 109 | 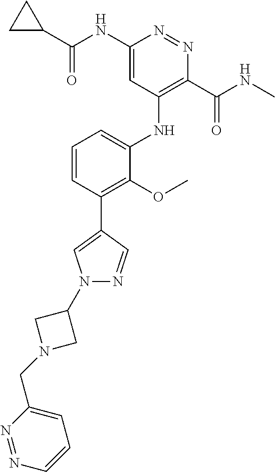 [SMILES: CNC(=O)c1nnc(NC(=O)C2CC2)cc1Nc1cccc(-c2cnn(C3CN(Cc4cccnn4)C3)c2)c1OC] | 555.1 [M + H]+ | ¹H NMR (400 MHz, DMSO) δ 11.29 (s, 1H), 10.96 (s, 1H), 9.19-9.08 (m, 2H), 8.37 (s, 1H), 8.14 (s, 1H), 8.02 (s, 1H), 7.73- 7.63 (m, 2H), 7.47 (dd, J = 7.8, 1.6 Hz, 1H), 7.29 (dd, J = 7.9, 1.6 Hz, 1H), 7.19 (t, J = 7.9 Hz, 1H), 5.12 (p, J = 6.9 Hz, 1H), 4.04 (s, 2H), 3.81 (t, J = 7.7 Hz, 2H), 3.64 (t, 2H), 3.58 (s, 3H), 2.85 (d, J = 4.8 Hz, 3H), 2.07 (tt, J = 7.0, 5.3 Hz, 1H), 0.86-0.73 (m, 4H). |
| 110 | 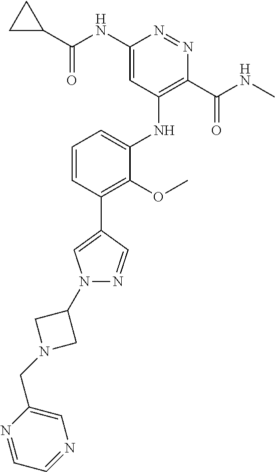 [SMILES: CNC(=O)c1nnc(NC(=O)C2CC2)cc1Nc1cccc(-c2cnn(C3CN(Cc4cnccn4)C3)c2)c1OC] | 555.4 [M + H]+ | ¹H NMR (500 MHz, DMSO) δ 11.29 (s, 1H), 10.96 (s, 1H), 9.14 (q, J = 4.8 Hz, 1H), 8.66 (d, J = 1.5 Hz, 1H), 8.58 (dd, J = 2.6, 1.5 Hz, 1H), 8.54 (d, J = 2.6 Hz, 1H), 8.37 (d, J = 0.8 Hz, 1H), 8.13 (s, 1H), 8.01 (d, J = 0.7 Hz, 1H), 7.46 (dd, J = 7.8, 1.5 Hz, 1H), 7.29 (dd, J = 7.9, 1.5 Hz, 1H), 7.19 (t, J = 7.9 Hz, 1H), 5.12 (p, J = 6.9 Hz, 1H), 3.90 (s, 2H), 3.83 (td, J = 7.1, 1.7 Hz, 2H), 3.62 (td, J = 6.7, 1.6 Hz, 2H), 3.58 (s, 3H), 2.85 (d, J = 4.8 Hz, 3H), 2.11-2.02 (m, 1H), 0.83-0.76 (m, 4H). |
| 111 | 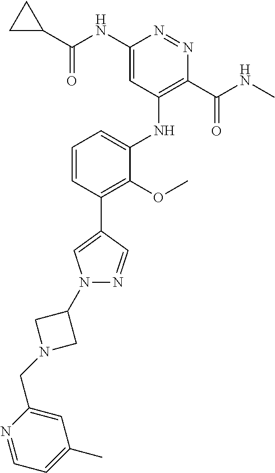 [SMILES: CNC(=O)c1nnc(NC(=O)C2CC2)cc1Nc1cccc(-c2cnn(C3CN(Cc4cc(C)ccn4)C3)c2)c1OC] | 568.5 [M + H]+ | ¹H NMR (500 MHz, DMSO) δ 11.29 (s, 1H), 10.96 (s, 1H), 9.14 (q, J = 4.7 Hz, 1H), 8.36 (d, J = 0.8 Hz, 1H), 8.33 (d, J = 5.0 Hz, 1H), 8.13 (s, 1H), 8.01 (s, 1H), 7.47 (dd, J = 7.8, 1.6 Hz, 1H), 7.29 (dd, J = 7.9, 1.6 Hz, 1H), 7.24- 7.22 (m, 1H), 7.19 (t, J = 7.9 Hz, 1H), 7.08 (d, J = 5.0 Hz, 1H), 5.10 (p, J = 6.9 Hz, 1H), 3.89-3.74 (m, 4H), 3.66-3.53 (m, 5H), 2.85 (d, J = 4.9 Hz, 3H), 2.31 (s, 3H), 2.10- 2.02 (m, 1H), 0.83-0.75 (m, 4H). |
| 122 | 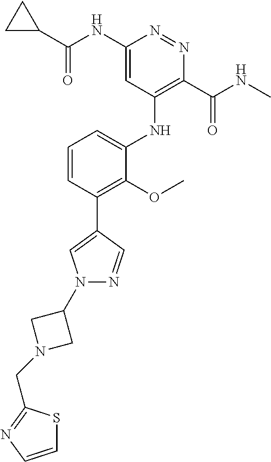 [SMILES: CNC(=O)c1nnc(NC(=O)C2CC2)cc1Nc1cccc(-c2cnn(C3CN(Cc4nccs4)C3)c2)c1OC] | 560.4 [M + H]+ | ¹H NMR (500 MHz, DMSO) δ 11.29 (s, 1H), 10.96 (s, 1H), 9.14 (q, J = 4.7 Hz, 1H), 8.38 (s, 1H), 8.13 (s, 1H), 8.02 (s, 1H), 7.73 (d, J = 3.3 Hz, 1H), 7.65 (d, J = 3.3 Hz, 1H), 7.47 (dd, J = 7.8, 1.5 Hz, 1H), 7.29 (dd, J = 7.9, 1.6 Hz, 1H), 7.19 (t, J = 7.9 Hz, 1H), 5.15 (p, J = 7.0 Hz, 1H), 4.06 (s, 2H), 3.87 (td, J = 7.2, 1.7 Hz, 2H), 3.63 (td, J = 6.7, 1.6 Hz, 2H), 3.59 (s, 3H), 2.86 (d, J = 4.9 Hz, 3H), 2.10-2.03 (m, 1H), 0.84-0.77 (m, 4H). |
| 123 | 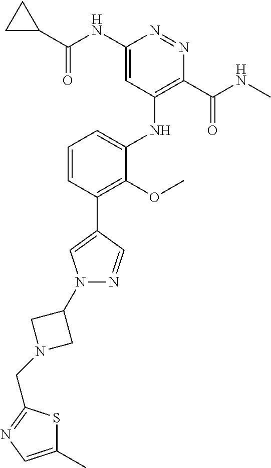 [SMILES: CNC(=O)c1nnc(NC(=O)C2CC2)cc1Nc1cccc(-c2cnn(C3CN(Cc4ncc(C)s4)C3)c2)c1OC] | 574.4 [M + H]+ | ¹H NMR (500 MHz, DMSO) δ 11.29 (s, 1H), 10.96 (s, 1H), 9.14 (q, J = 4.8 Hz, 1H), 8.37 (d, J = 0.8 Hz, 1H), 8.14 (s, 1H), 8.02 (d, J = 0.7 Hz, 1H), 7.46 (dd, J = 7.9, 1.6 Hz, 1H), 7.38 (d, J = 1.3 Hz, 1H), 7.29 (dd, J = 8.0, 1.5 Hz, 1H), 7.19 (t, J = 7.9 Hz, 1H), 5.13 (p, J = 7.0 Hz, 1H), 3.97 (s, 2H), 3.85 (t, J = 7.5 Hz, 2H), 3.65-3.56 (m, 5H), 2.86 (d, J = 4.9 Hz, 3H), 2.40 (d, J = 1.4 Hz, 3H), 2.10-2.03 (m, 1H), 0.83-0.77 (m, 4H). |
| 124 | 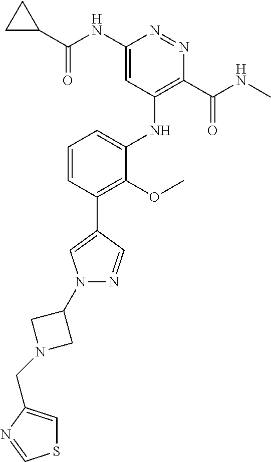 [SMILES: CNC(=O)c1nnc(NC(=O)C2CC2)cc1Nc1cccc(-c2cnn(C3CN(Cc4cscn4)C3)c2)c1OC] | 560.4 [M + H]+ | ¹H NMR (500 MHz, DMSO) δ 11.29 (s, 1H), 10.96 (s, 1H), 9.14 (q, J = 4.8 Hz, 1H), 9.05 (d, J = 2.0 Hz, 1H), 8.35 (d, J = 0.8 Hz, 1H), 8.13 (s, 1H), 8.00 (d, J = 0.7 Hz, 1H), 7.51 (d, J = 2.0 Hz, 1H), 7.46 (dd, J = 7.8, 1.5 Hz, 1H), 7.29 (dd, J = 7.9, 1.6 Hz, 1H), 7.19 (t, J = 7.9 Hz, 1H), 5.08 (p, J = 7.0 Hz, 1H), 3.86 (s, 2H), 3.80 (t, J = 7.3 Hz, 2H), 3.63-3.53 (m, 5H), 2.85 (d, J = 4.8 Hz, 3H), 2.10-2.03 (m, 1H), 0.83-0.78 (m, 4H). |
| 125 | 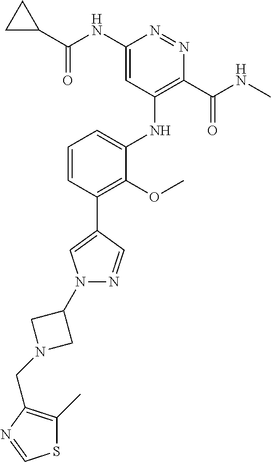 [SMILES: CNC(=O)c1nnc(NC(=O)C2CC2)cc1Nc1cccc(-c2cnn(C3CN(Cc4ncsc4C)C3)c2)c1OC] | 574.4 [M + H]+ | ¹H NMR (500 MHz, DMSO) δ 11.29 (s, 1H), 10.96 (s, 1H), 9.14 (q, J = 4.7 Hz, 1H), 8.88 (s, 1H), 8.34 (d, J = 0.8 Hz, 1H), 8.13 (s, 1H), 8.01 (s, 1H), 7.46 (dd, J = 7.9, 1.5 Hz, 1H), 7.29 (dd, J = 7.9, 1.6 Hz, 1H), 7.19 (t, J = 7.9 Hz, 1H), 5.08 (p, J = 7.0 Hz, 1H), 3.86 (s, 2H), 3.75 (t, J = 7.3 Hz, 2H), 3.58 (s, 3H), 3.51 (t, J = 7.4 Hz, 2H), 2.85 (d, J = 4.8 Hz, 3H), 2.36 (s, 3H), 2.10-2.04 (m, 1H), 0.84-0.76 (m, 4H). |
| 126 | 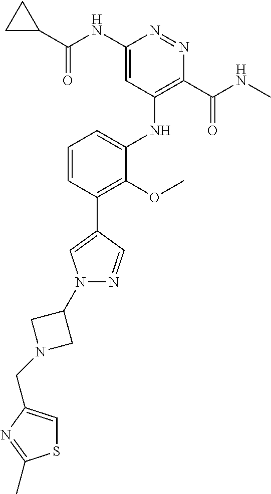 [SMILES: CNC(=O)c1nnc(NC(=O)C2CC2)cc1Nc1cccc(-c2cnn(C3CN(Cc4csc(C)n4)C3)c2)c1OC] | 574.4 [M + H]+ | ¹H NMR (500 MHz, DMSO) δ 11.29 (s, 1H), 10.96 (s, 1H), 9.14 (q, J = 4.8 Hz, 1H), 8.34 (s, 1H), 8.13 (s, 1H), 8.00 (s, 1H), 7.46 (dd, J = 7.8, 1.6 Hz, 1H), 7.29 (dd, J = 7.9, 1.6 Hz, 1H), 7.25 (s, 1H), 7.19 (t, J = 7.9 Hz, 1H), 5.07 (p, J = 6.9 Hz, 1H), 3.82-3.72 (m, 4H), 3.63-3.51 (m, 5H), 2.85 (d, J = 4.9 Hz, 3H), 2.62 (s, 3H), 2.10- 2.04 (m, 1H), 0.83-0.78 (m, 4H). |
| 128 | 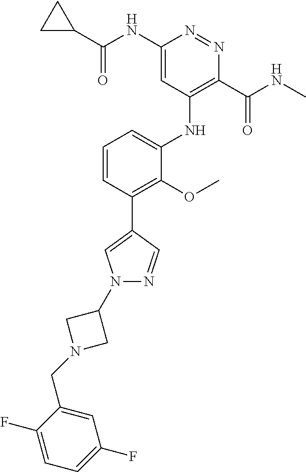 [SMILES: CNC(=O)c1nnc(NC(=O)C2CC2)cc1Nc1cccc(-c2cnn(C3CN(Cc4cc(F)ccc4F)C3)c2)c1OC] | 589.4 [M + H]+ | ¹H NMR (400 MHz, DMSO) δ 11.30 (s, 1H), 10.96 (s, 1H), 9.18-9.12 (m, 1H), 8.36 (d, J = 0.8 Hz, 1H), 8.14 (s, 1H), 8.01 (s, 1H), 7.46 (dd, J = 7.8, 1.6 Hz, 1H), 7.31-7.11 (m, 5H), 5.14-5.04 (m, 1H), 3.82-3.69 (m, 4H), 3.63-3.49 (m, 5H), 2.85 (d, J = 4.8 Hz, 3H), 2.11- 2.02 (m, 1H), 0.84-0.76 (m, 4H). |
| 129 | 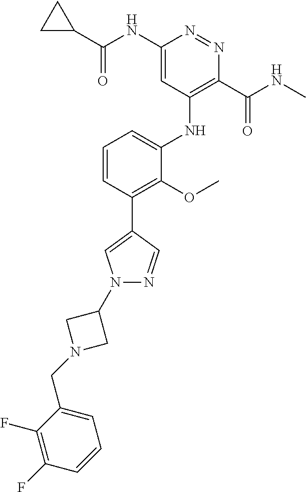 [SMILES: CNC(=O)c1nnc(NC(=O)C2CC2)cc1Nc1cccc(-c2cnn(C3CN(Cc4cccc(F)c4F)C3)c2)c1OC] | 589.5 [M + H]+ | ¹H NMR (500 MHz, DMSO) δ 11.29 (s, 1H), 10.96 (s, 1H), 9.14 (q, J = 4.7 Hz, 1H), 8.35 (d, J = 0.8 Hz, 1H), 8.13 (s, 1H), 8.01 (d, J = 0.7 Hz, 1H), 7.46 (dd, J = 7.8, 1.6 Hz, 1H), 7.36-7.23 (m, 3H), 7.21-7.16 (m, 2H), 5.08 (p, J = 6.9 Hz, 1H), 3.79 (s, 2H), 3.75 (t, J = 7.3 Hz, 2H), 3.58 (s, 3H), 3.55 (t, J = 7.2 Hz, 2H), 2.85 (d, J = 4.8 Hz, 3H), 2.11-2.03 (m, 1H), 0.84-0.76 (m, 4H). |
| 130 | 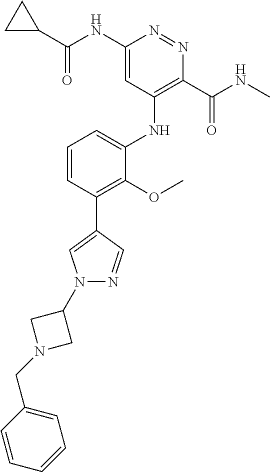 [SMILES: CNC(=O)c1nnc(NC(=O)C2CC2)cc1Nc1cccc(-c2cnn(C3CN(Cc4ccccc4)C3)c2)c1OC] | 553.4 [M + H]+ | ¹H NMR (400 MHz, DMSO) δ 11.30 (s, 1H), 10.96 (s, 1H), 9.15 (q, J = 4.6 Hz, 1H), 8.34 (d, J = 0.7 Hz, 1H), 8.14 (s, 1H), 8.01 (s, 1H), 7.46 (dd, J = 7.8, 1.6 Hz, 1H), 7.34- 7.16 (m, 7H), 5.14-5.02 (m, 1H), 3.80-3.63 (m, 4H), 3.58 (s, 3H), 3.54- 3.43 (m, 2H), 2.85 (d, J = 4.8 Hz, 3H), 2.12-2.02 (m, 1H), 0.85-0.76 (m, 4H). |
| 141 | 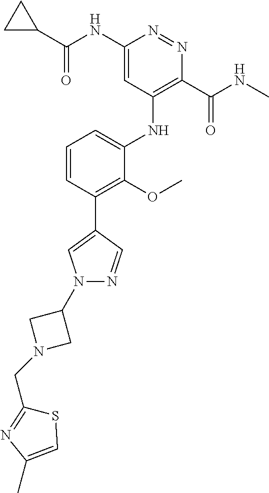 [SMILES: CNC(=O)c1nnc(NC(=O)C2CC2)cc1Nc1cccc(-c2cnn(C3CN(Cc4nc(C)cs4)C3)c2)c1OC] | 574.3 [M + H]+ | ¹H NMR (500 MHz, DMSO) δ 11.29 (s, 1H), 10.96 (s, 1H), 9.14 (q, J = 4.8 Hz, 1H), 8.37 (s, 1H), 8.13 (s, 1H), 8.02 (s, 1H), 7.46 (dd, J = 7.8, 1.6 Hz, 1H), 7.29 (dd, J = 8.0, 1.6 Hz, 1H), 7.19 (t, J = 7.9 Hz, 1H), 7.16 (d, J = 1.2 Hz, 1H), 5.14 (p, J = 7.0 Hz, 1H), 4.00 (s, 2H), 3.87 (t, J = 7.6 Hz, 2H), 3.62 (t, J = 7.2 Hz, 2H), 3.59 (s, 3H), 2.86 (d, J = 4.7 Hz, 3H), 2.32 (d, J = 1.2 Hz, 3H), 2.11-2.02 (m, 1H), 0.85- 0.75 (m, 4H). |
| 142 | 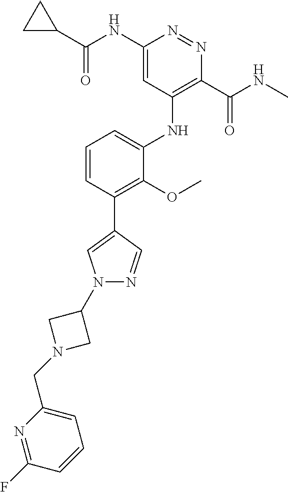 [SMILES: CNC(=O)c1nnc(NC(=O)C2CC2)cc1Nc1cccc(-c2cnn(C3CN(Cc4cccc(F)n4)C3)c2)c1OC] | 572.4 [M + H]+ | ¹H NMR (500 MHz, DMSO) δ 11.29 (s, 1H), 10.96 (s, 1H), 9.14 (q, J = 4.7 Hz, 1H), 8.37 (d, J = 0.7 Hz, 1H), 8.13 (s, 1H), 8.01 (d, J = 0.7 Hz, 1H), 7.96 (td, J = 8.4, 7.4 Hz, 1H), 7.47 (dd, J = 7.9, 1.6 Hz, 1H), 7.35 (dd, J = 7.3, 2.6 Hz, 1H), 7.29 (dd, J = 7.9, 1.5 Hz, 1H), 7.19 (t, J = 7.9 Hz, 1H), 7.04 (dd, J = 8.1, 2.6 Hz, 1H), 5.11 (p, J = 6.9 Hz, 1H), 3.82 (td, J = 7.1, 1.7 Hz, 2H), 3.78 (s, 2H), 3.61-3.55 (m, 5H), 2.85 (d, J = 4.9 Hz, 3H), 2.10-2.02 (m, 1H), 0.83- 0.77 (m, 4H). |
| 143 | 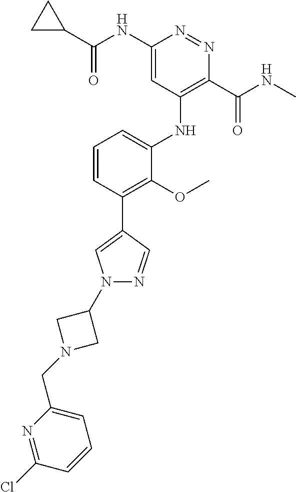 [SMILES: CNC(=O)c1nnc(NC(=O)C2CC2)cc1Nc1cccc(-c2cnn(C3CN(Cc4cccc(Cl)n4)C3)c2)c1OC] | 588.3 [M + H]+ | ¹H NMR (500 MHz, DMSO) δ 11.29 (s, 1H), 10.96 (s, 1H), 9.14 (q, J = 4.8 Hz, 1H), 8.37 (s, 1H), 8.13 (s, 1H), 8.01 (s, 1H), 7.84 (t, J = 7.7 Hz, 1H), 7.47 (dd, J = 7.8, 1.5 Hz, 1H), 7.42 (d, J = 7.5 Hz, 1H), 7.38 (d, J = 7.9 Hz, 1H), 7.29 (dd, J = 7.9, 1.5 Hz, 1H), 7.19 (t, J = 7.9 Hz, 1H), 5.11 (p, J = 6.9 Hz, 1H), 3.86-3.77 (m, 4H), 3.64-3.54 (m, 5H), 2.85 (d, J = 4.8 Hz, 3H), 2.10- 2.03 (m, 1H), 0.84-0.77 (m, 4H). |
| 144 | 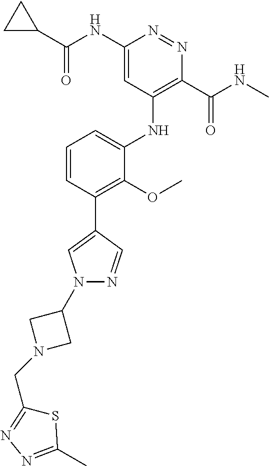 [SMILES: CNC(=O)c1nnc(NC(=O)C2CC2)cc1Nc1cccc(-c2cnn(C3CN(Cc4nnc(C)s4)C3)c2)c1OC] | 575.0 [M + H]+ | ¹H NMR (500 MHz, DMSO) δ 11.30 (s, 1H), 10.97 (s, 1H), 9.15 (d, J = 4.8 Hz, 1H), 8.38 (s, 1H), 8.15 (d, J = 2.9 Hz, 1H), 8.03 (s, 1H), 7.47 (dd, J = 7.8, 1.6 Hz, 1H), 7.30 (dd, J = 7.8, 1.5 Hz, 1H), 7.20 (t, J = 7.9 Hz, 1H), 5.14 (p, J = 6.9 Hz, 1H), 4.13 (s, 2H), 3.84 (td, J = 7.2, 1.7 Hz, 2H), 3.64 (td, J = 6.6, 1.6 Hz, 2H), 3.59 (s, 3H), 2.86 (d, J = 4.8 Hz, 3H), 2.70 (s, 3H), 2.12-2.02 (m, 1H), 0.81 (dd, J = 6.3, 3.5 Hz, 4H). |
| 145 | 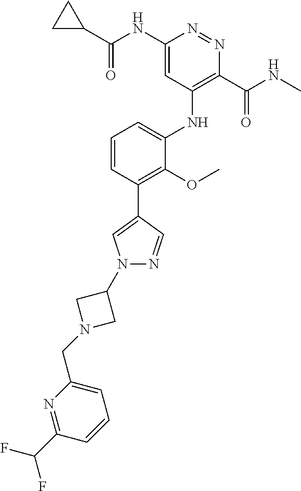 [SMILES: CNC(=O)c1nnc(NC(=O)C2CC2)cc1Nc1cccc(-c2cnn(C3CN(Cc4cccc(C(F)F)n4)C3)c2)c1OC] | 604.4 [M + H]+ | ¹H NMR (500 MHz, DMSO) δ 11.29 (s, 1H), 10.96 (s, 1H), 9.14 (q, J = 4.7 Hz, 1H), 8.37 (d, J = 0.8 Hz, 1H), 8.13 (s, 1H), 8.03-8.01 (m, 1H), 7.98 (t, J = 7.8 Hz, 1H), 7.58 (t, J = 8.0 Hz, 2H), 7.47 (dd, J = 7.8, 1.6 Hz, 1H), 7.29 (dd, J = 7.9, 1.5 Hz, 1H), 7.19 (t, J = 7.9 Hz, 1H), 6.92 (t, J = 55.0 Hz, 1H), 5.13 (p, J = 6.9 Hz, 1H), 3.90 (s, 2H), 3.86-3.79 (m, 2H), 3.66-3.59 (m, 2H), 3.58 (s, 3H), 2.85 (d, J = 4.9 Hz, 3H), 2.12-2.02 (m, 1H), 0.85-0.76 (m, 4H). |
| 148 | 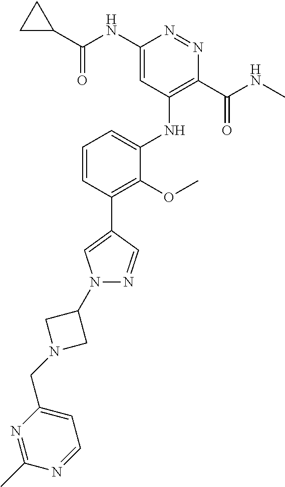 | 569.4 [M + H]+ | ¹H NMR (500 MHz, DMSO) δ 11.29 (s, 1H), 10.96 (s, 1H), 9.14 (q, J = 4.8 Hz, 1H), 8.63 (d, J = 5.2 Hz, 1H), 8.38 (d, J = 0.8 Hz, 1H), 8.13 (s, 1H), 8.02 (s, 1H), 7.47 (dd, J = 7.9, 1.6 Hz, 1H), 7.33- 7.27 (m, 2H), 7.19 (t, J = 7.9 Hz, 1H), 5.13 (p, J = 6.9 Hz, 1H), 3.85 (t, J = 7.3 Hz, 2H), 3.81 (s, 2H), 3.64- 3.53 (m, 5H), 2.85 (d, J = 4.9 Hz, 3H), 2.58 (s, 3H), 2.10-2.02 (m, 1H), 0.84- 0.72 (m, 4H). |
| 152 | 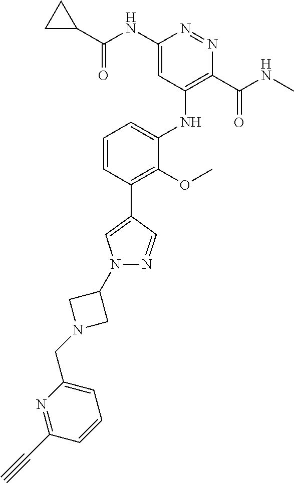 [SMILES: C#Cc1cccc(CN2CC(n3cc(-c4cccc(Nc5cc(NC(=O)C6CC6)nnc5C(=O)NC)c4OC)cn3)C2)n1] | 578.5 [M + H]+ | ¹H NMR (500 MHz, DMSO) δ 11.29 (s, 1H), 10.96 (s, 1H), 9.17-9.11 (m, 1H), 8.37 (d, J = 0.8 Hz, 1H), 8.14 (s, 1H), 8.01 (s, 1H), 7.79 (t, J = 7.8 Hz, 1H), 7.47 (dd, J = 7.8, 1.5 Hz, 1H), 7.44 (d, J = 7.8 Hz, 2H), 7.29 (dd, J = 7.9, 1.5 Hz, 1H), 7.19 (t, J = 7.9 Hz, 1H), 5.11 (p, J = 6.9 Hz, 1H), 4.29 (s, 1H), 3.85- 3.77 (m, 4H), 3.63-3.55 (m, 5H), 2.86 (d, J = 4.9 Hz, 3H), 2.11-2.03 (m, 1H), 0.84-0.76 (m, 4H). |
| 169 | 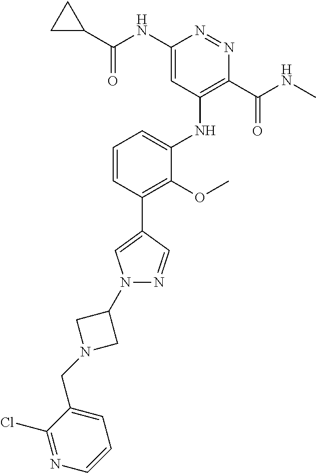 [SMILES: CNC(=O)c1nnc(NC(=O)C2CC2)cc1Nc1cccc(-c2cnn(C3CN(Cc4cccnc4Cl)C3)c2)c1OC] | 588.1 [M + H]+ | |
| 173 | 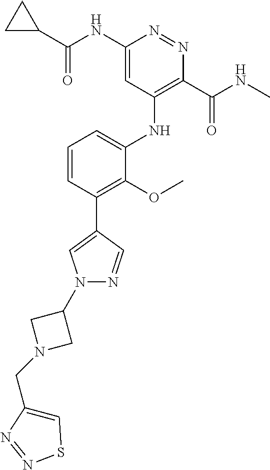 [SMILES: CNC(=O)c1nnc(NC(=O)C2CC2)cc1Nc1cccc(-c2cnn(C3CN(Cc4csnn4)C3)c2)c1OC] | 561.5 [M + H]+ | ¹H NMR (500 MHz, DMSO) δ 11.29 (s, 1H), 10.96 (s, 1H), 9.14 (q, J = 4.8 Hz, 1H), 9.07 (s, 1H), 8.35 (d, J = 0.8 Hz, 1H), 8.13 (s, 1H), 8.01 (d, J = 0.8 Hz, 1H), 7.46 (dd, J = 7.8, 1.6 Hz, 1H), 7.29 (dd, J = 7.9, 1.5 Hz, 1H), 7.19 (t, J = 7.9 Hz, 1H), 5.10 (p, J = 7.0 Hz, 1H), 4.22 (s, 2H), 3.81 (t, J = 7.4 Hz, 2H), 3.64 (t, J = 7.5 Hz, 2H), 3.58 (s, 3H), 2.85 (d, J = 4.8 Hz, 3H), 2.10-2.03 (m, 1H), 0.85-0.75 (m, 4H). |
| 200 | 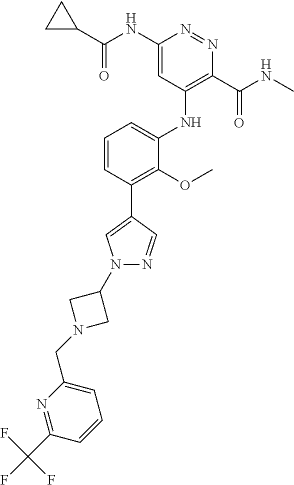 [SMILES: CNC(=O)c1nnc(NC(=O)C2CC2)cc1Nc1cccc(-c2cnn(C3CN(Cc4cccc(C(F)(F)F)n4)C3)c2)c1OC] | 622.5 [M + H]+ | ¹H NMR (500 MHz, DMSO) δ 11.29 (s, 1H), 10.96 (s, 1H), 9.14 (q, J = 4.8 Hz, 1H), 8.38 (d, J = 0.8 Hz, 1H), 8.13 (s, 1H), 8.07 (t, J = 7.8 Hz, 1H), 8.02 (s, 1H), 7.78 (d, J = 7.7 Hz, 1H), 7.73 (d, J = 7.9 Hz, 1H), 7.47 (dd, J = 7.9, 1.6 Hz, 1H), 7.29 (dd, J = 7.9, 1.5 Hz, 1H), 7.19 (t, J = 7.9 Hz, 1H), 5.13 (p, J = 6.9 Hz, 1H), 3.93 (s, 2H), 3.88-3.81 (m, 2H), 3.66- 3.60 (m, 2H), 3.58 (s, 3H), 2.85 (d, J = 4.8 Hz, 3H), 2.10-2.02 (m, 1H), 0.83- 0.75 (m, 4H). |
| 201 | 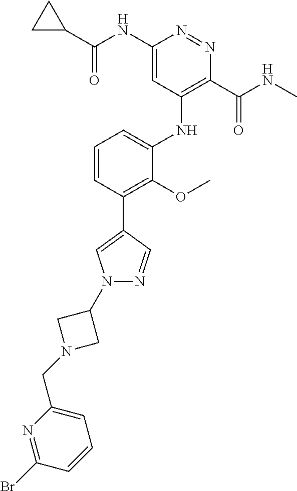 [SMILES: CNC(=O)c1nnc(NC(=O)C2CC2)cc1Nc1cccc(-c2cnn(C3CN(Cc4cccc(Br)n4)C3)c2)c1OC] | 632.3 [M + H]+ | ¹H NMR (500 MHz, DMSO) δ 11.29 (s, 1H), 10.96 (s, 1H), 9.14 (q, J = 4.8 Hz, 1H), 8.37 (d, J = 0.8 Hz, 1H), 8.13 (s, 1H), 8.01 (d, J = 0.7 Hz, 1H), 7.73 (t, J = 7.7 Hz, 1H), 7.51 (d, J = 7.9 Hz, 1H), 7.48-7.41 (m, 2H), 7.29 (dd, J = 7.9, 1.5 Hz, 1H), 7.19 (t, J = 7.9 Hz, 1H), 5.11 (p, J = 6.9 Hz, 1H), 3.85-3.77 (m, 4H), 3.61- 3.55 (m, 5H), 2.85 (d, J = 4.8 Hz, 3H), 2.10-2.03 (m, 1H), 0.84-0.76 (m, 4H). |
| 212 | 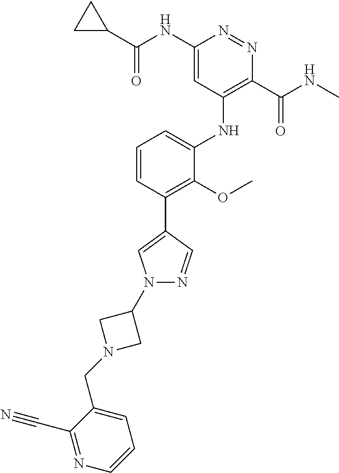 | 597.4 [M + H]+ | ¹H NMR (500 MHz, DMSO) δ 11.29 (s, 1H), 10.96 (s, 1H), 9.14 (dd, J = 5.0 Hz, 1H), 8.47 (d, J = 4.6 Hz, 1H), 8.37 (s, 1H), 8.13 (s, 1H), 8.01 (s, 1H), 7.99 (d, J = 8.3 Hz, 1H), 7.53 (dd, J = 7.9, 4.7 Hz, 1H), 7.46 (dd, J = 7.9, 1.6 Hz, 1H), 7.29 (dd, J = 7.8, 1.6 Hz, 1H), 7.19 (t, J = 7.9 Hz, 1H), 5.20-5.03 (m, 1H), 4.13 (s, 2H), 3.83- 3.75 (m, 2H), 3.61-3.50 (m, 5H), 2.85 (d, J = 4.8 Hz, 3H), 2.10-2.03 (m, 1H), 0.85-0.73 (m, 4H). |
| 213 | 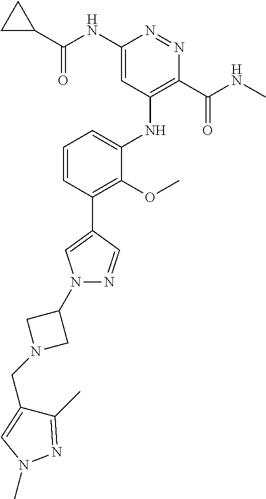 [SMILES: CNC(=O)c1nnc(NC(=O)C2CC2)cc1Nc1cccc(-c2cnn(C3CN(Cc4cn(C)nc4C)C3)c2)c1OC] | 571.5 [M + H]+ | ¹H NMR (500 MHz, DMSO) δ 11.29 (s, 1H), 10.96 (s, 1H), 9.17-9.12 (m, 1H), 8.31 (s, 1H), 8.13 (s, 1H), 8.00 (s, 1H), 7.48 (s, 1H), 7.45 (dd, J = 7.8, 1.6 Hz, 1H), 7.28 (dd, J = 7.9, 1.5 Hz, 1H), 7.19 (t, J = 7.9 Hz, 1H), 5.06-4.97 (m, 1H), 3.69 (s, 3H), 3.64 (t, J = 7.0 Hz, 2H), 3.57 (s, 3H), 3.45 (s, 2H), 3.41- 3.37 (m, 2H), 2.85 (d, J = 4.8 Hz, 3H), 2.13-2.04 (m, 4H), 0.82-0.77 (m, 4H). |
| 215 | 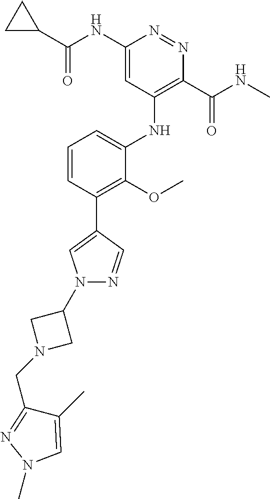 [SMILES: CNC(=O)c1nnc(NC(=O)C2CC2)cc1Nc1cccc(-c2cnn(C3CN(Cc4nn(C)cc4C)C3)c2)c1OC] | 571.5 [M + H]+ | ¹H NMR (500 MHz, DMSO) δ 11.29 (s, 1H), 10.96 (s, 1H), 9.14 (q, J = 4.7 Hz, 1H), 8.29 (s, 1H), 8.13 (s, 1H), 7.99 (s, 1H), 7.45 (dd, J = 7.9, 1.5 Hz, 1H), 7.35 (s, 1H), 7.28 (dd, J = 7.9, 1.5 Hz, 1H), 7.19 (t, J = 7.9 Hz, 1H), 5.00 (p, J = 7.0 Hz, 1H), 3.70 (s, 3H), 3.65 (t, J = 7.2 Hz, 2H), 3.59-3.54 (m, 5H), 3.47 (t, J = 7.5 Hz, 2H), 2.86 (d, J = 4.8 Hz, 3H), 2.11-2.02 (m, 1H), 1.98 (s, 3H), 0.83-0.74 (m, 4H). |
| 216 | 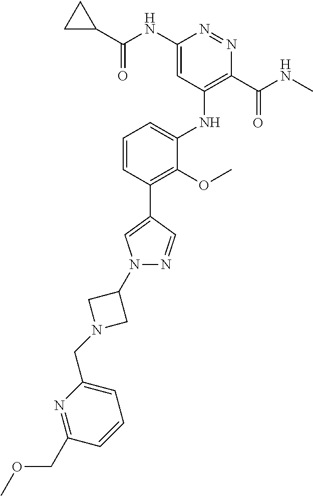 | 598.5 [M + H]+ | ¹H NMR (500 MHz, DMSO) δ 11.29 (s, 1H), 10.96 (s, 1H), 9.14 (dd, J = 5.0 Hz, 1H), 8.36 (s, 1H), 8.13 (s, 1H), 8.01 (s, 1H), 7.77 (t, J = 7.7 Hz, 1H), 7.47 (dd, J = 7.8, 1.5 Hz, 1H), 7.33-7.26 (m, 3H), 7.19 (t, J = 7.9 Hz, 1H), 5.17-5.07 (m, 1H), 4.46 (s, 2H), 3.88-3.77 (m, 4H), 3.65-3.51 (m, 5H), 3.35 (s, 3H), 2.85 (d, J = 4.8 Hz, 3H), 2.10-2.03 (m, 1H), 0.84-0.75 (m, 4H). |
| 228 | 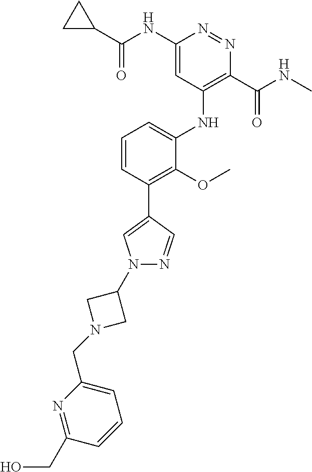 [SMILES: CNC(=O)c1nnc(NC(=O)C2CC2)cc1Nc1cccc(-c2cnn(C3CN(Cc4cccc(CO)n4)C3)c2)c1OC] | 584.3 [M + H]+ | ¹H NMR (400 MHz, DMSO) & 11.30 (s, 1H), 10.96 (s, 1H), 9.15 (q, J = 4.8 Hz, 1H), 8.36 (d, J = 0.7 Hz, 1H), 8.14 (s, 1H), 8.01 (d, J = 0.7 Hz, 1H), 7.76 (t, J = 7.7 Hz, 1H), 7.47 (dd, J = 7.8, 1.6 Hz, 1H), 7.33 (d, J = 7.7 Hz, 1H), 7.29 (dd, J = 8.0, 1.6 Hz, 1H), 7.25 (d, J = 7.7 Hz, 1H), 7.19 (t, J = 7.9 Hz, 1H), 5.35 (t, J = 5.9 Hz, 1H), 5.11 (p, J = 7.0 Hz, 1H), 4.53 (d, J = 5.9 Hz, 2H), 3.85-3.76 (m, 4H), 3.63-3.53 (m, 5H), 2.85 (d, J = 4.8 Hz, 3H), 2.12- 2.01 (m, 1H), 0.84-0.74 (m, 4H). |
| 241 | 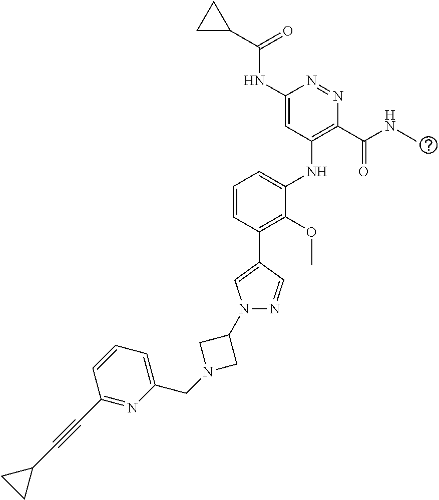 [SMILES: CNC(=O)c1nnc(NC(=O)C2CC2)cc1Nc1cccc(-c2cnn(C3CN(Cc4cccc(C#CC5CC5)n4)C3)c2)c1OC] | 618.3 [M + H]+ | ¹H NMR (400 MHz, DMSO-d6) 11.32 (s, 1H), 10.99 (s, 1H), 9.18 (q, J = 4.8 Hz, 1H), 8.38 (s, 1H), 8.16 (s, 1H), 8.03 (s, 1H), 7.73 (t, J = 7.8 Hz, 1H), 7.48 (dd, J = 7.8, 1.6 Hz, 1H), 7.36-7.27 (m, 3H), 7.20 (t, J = 7.9 Hz, 1H), 5.12 (t, J = 6.9 Hz, 1H), 3.84-3.74 (m, 4H), 3.64- 3.51 (m, 5H), 2.87 (d, J = 4.8 Hz, 3H), 2.13-2.04 (m, 1H), 1.57 (tt, J = 8.3, 5.0 Hz, 1H), 0.97-0.88 (m, 2H), 0.86-0.72 (m, 6H). |
| 242 | 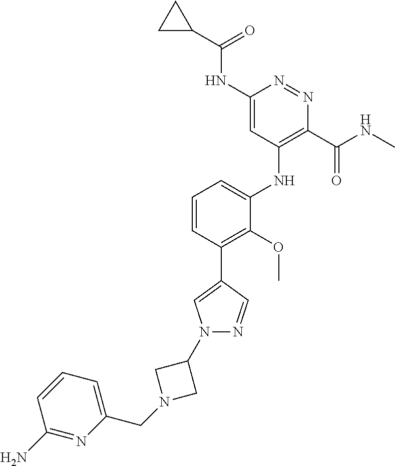 [SMILES: CNC(=O)c1nnc(NC(=O)C2CC2)cc1Nc1cccc(-c2cnn(C3CN(Cc4cccc(N)n4)C3)c2)c1OC] | 569.4 [M + H]+ | ¹H NMR (400 MHz, DMSO-d6) 11.32 (s, 1H), 10.98 (s, 1H), 9.18 (q, J = 4.7 Hz, 1H), 8.36 (s, 1H), 8.16 (s, 1H), 8.03 (s, 1H), 7.51-7.45 (m, 1H), 7.39- 7.27 (m, 2H), 7.21 (t, J = 7.9 Hz, 1H), 6.51 (d, J = 7.1 Hz, 1H), 6.29 (t, J = 8.9 Hz, 1H), 5.83 (s, 2H), 5.15- 5.02 (m, 1H), 4.31 (d, J = 5.8 Hz, 1H), 3.82-3.74 (m, 2H), 3.59 (d, 4H), 3.57- 3.48 (m, 2H), 2.87 (d, J = 4.8 Hz, 3H), 2.14-2.03 (m, 1H), 0.88-0.79 (m, 4H). |
| 243 | 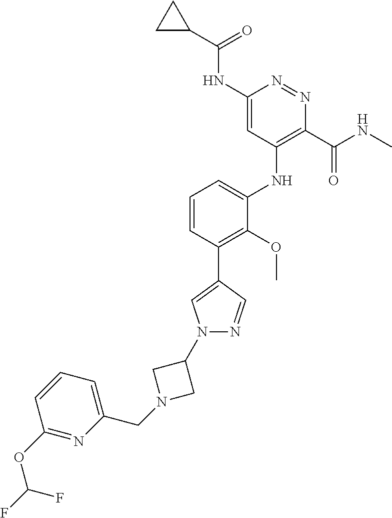 [SMILES: CNC(=O)c1nnc(NC(=O)C2CC2)cc1Nc1cccc(-c2cnn(C3CN(Cc4cccc(OC(F)F)n4)C3)c2)c1OC] | 620.3 [M + H]+ | ¹H NMR (400 MHz, DMSO-d6) 11.34 (s, 1H), 11.00 (s, 1H), 9.20 (d, J = 5.0 Hz, 1H), 8.40 (s, 1H), 8.17 (s, 1H), 8.05 (s, 1H), 7.97-7.84 (m, 1H), 7.71 (s, 2H), 7.55-7.46 (m, 1H), 7.33-7.27 (m, 2H), 7.22 (t, J = 7.9 Hz, 1H), 6.97 (d, J = 8.1 Hz, 1H), 5.14 (p, J = 6.9 Hz, 1H), 3.86 (t, J = 7.5 Hz, 2H), 3.80 (s, 2H), 3.67- 3.58 (m, 5H), 2.87 (d, J = 4.8 Hz, 3H), 2.13-2.04 (m, 1H), 0.83 (dd, J = 6.4, 3.4 Hz, 4H). |
| 248 | 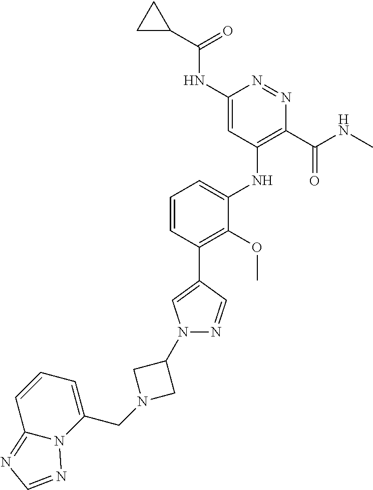 [SMILES: CNC(=O)c1nnc(NC(=O)C2CC2)cc1Nc1cccc(-c2cnn(C3CN(Cc4cccc5ncnn45)C3)c2)c1OC] | 594.1 [M + H]+ | ¹H NMR (500 MHz, DMSO) δ 11.29 (s, 1H), 10.96 (s, 1H), 9.19-9.11 (m, 1H), 8.52 (s, 1H), 8.41 (s, 1H), 8.14 (s, 1H), 8.03 (s, 1H), 7.78 (d, J = 8.8 Hz, 1H), 7.69 (dd, J = 8.9, 7.0 Hz, 1H), 7.47 (dd, J = 7.9, 1.6 Hz, 1H), 7.29 (dd, J = 7.9, 1.5 Hz, 1H), 7.24- 7.16 (m, 2H), 5.23-5.13 (m, 1H), 4.27 (s, 2H), 4.01- 3.91 (m, 2H), 3.79-3.69 (m, 2H), 3.59 (s, 3H), 2.86 (d, J = 4.8 Hz, 3H), 2.10- 2.02 (m, 1H), 0.85-0.71 (m, 4H). |
| 249 | 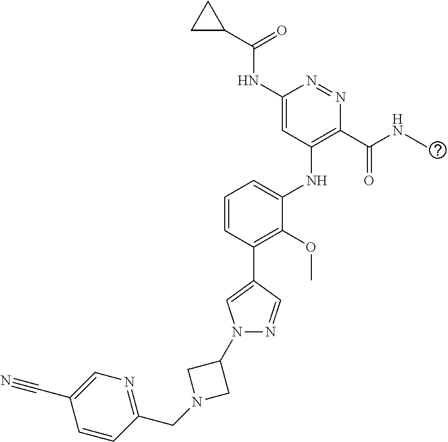 [SMILES: CNC(=O)c1nnc(NC(=O)C2CC2)cc1Nc1cccc(-c2cnn(C3CN(Cc4ccc(C#N)cn4)C3)c2)c1OC] | 578.2 [M + H]+ | ¹H NMR (formate salt) (500 MHz, DMSO) δ 11.29 (s, 1H), 10.96 (s, 1H), 9.14 (d, J = 5.0 Hz, 1H), 8.95 (d, J = 2.2 Hz, 1H), 8.38 (s, 1H), 8.28 (dd, J = 8.2, 2.2 Hz, 1H), 8.13 (d, J = 5.5 Hz, 2H), 8.02 (s, 1H), 7.62 (d, J = 8.2 Hz, 1H), 7.46 (dd, J = 7.8, 1.6 Hz, 1H), 7.29 (dd, J = 8.0, 1.6 Hz, 1H), 7.19 (t, J = 7.9 Hz, 1H), 5.13 (t, J = 7.0 Hz, 1H), 3.95 (s, 2H), 3.85 (s, 2H), 3.58 (s, 5H), 2.85 (d, J = 4.9 Hz, 3H), 2.06 (q, J = 7.1, 6.4 Hz, 1H), 0.80 (dq, J = 5.3, 3.2 Hz, 4H). |
| 250 | 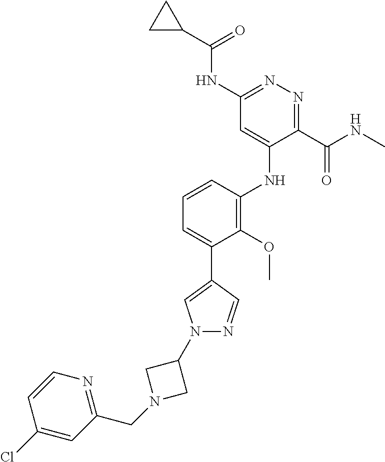 [SMILES: CNC(=O)c1nnc(NC(=O)C2CC2)cc1Nc1cccc(-c2cnn(C3CN(Cc4cc(Cl)ccn4)C3)c2)c1OC] | 588.0 [M + H]+ | ¹H NMR (500 MHz, DMSO) δ 11.29 (s, 1H), 10.96 (s, 1H), 9.14 (d, J = 5.0 Hz, 1H), 8.49 (d, J = 5.3 Hz, 1H), 8.37 (s, 1H), 8.14 (s, 1H), 8.02 (s, 1H), 7.51 (s, 1H), 7.47 (dd, J = 7.8, 1.6 Hz, 1H), 7.44-7.40 (m, 1H), 7.29 (dd, J = 7.9, 1.6 Hz, 1H), 7.19 (t, J = 7.9 Hz, 1H), 5.17-5.03 (m, 1H), 3.92-3.74 (m, 4H), 3.66- 3.52 (m, 5H), 2.85 (d, J = 4.8 Hz, 3H), 2.14-2.02 (m, 1H), 0.96-0.56 (m, 4H) |
| 251 | 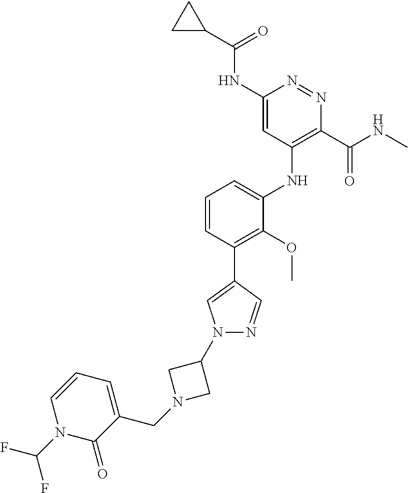 [SMILES: CNC(=O)c1nnc(NC(=O)C2CC2)cc1Nc1cccc(-c2cnn(C3CN(Cc4cccn(C(F)F)c4=O)C3)c2)c1OC] | 620.1 [M + H]+ | ¹H NMR (400 MHz, DMSO) δ 11.30 (s, 1H), 10.97 (s, 1H), 9.16 (d, J = 5.0 Hz, 1H), 8.39 (d, J = 0.8 Hz, 1H), 8.15 (s, 1H), 7.81 (d, J = 59.9 Hz, 1H), 7.69 (dd, J = 7.1, 1.9 Hz, 1H), 7.57-7.41 (m, 2H), 7.30 (dd, J = 7.9, 1.6 Hz, 1H), 7.20 (t, J = 7.9 Hz, 1H), 6.43 (t, J = 6.9 Hz, 1H), 5.11 (p, J = 6.9 Hz, 1H), 3.81 (td, J = 7.1, 1.6 Hz, 2H), 3.64- 3.51 (m, 7H), 2.86 (d, J = 4.8 Hz, 3H), 2.17-1.96 (m, 1H), 0.91-0.74 (m, 4H). |
| 254 | 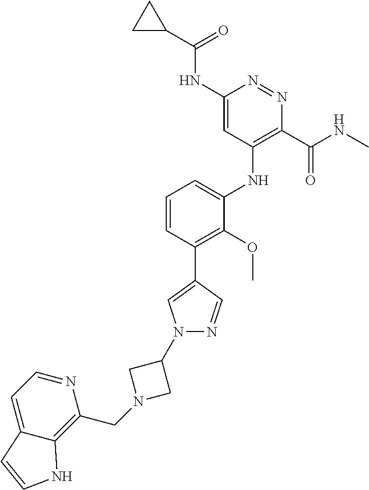 [SMILES: CNC(=O)c1nnc(NC(=O)C2CC2)cc1Nc1cccc(-c2cnn(C3CN(Cc4nccc5cc[nH]c45)C3)c2)c1OC] | 593.3 [M + H]+ | ¹H NMR (400 MHz, DMSO-d6) 11.34 (s, 1H), 11.00 (s, 1H), 9.20 (d, J = 5.0 Hz, 1H), 8.40 (s, 1H), 8.17 (s, 1H), 8.05 (s, 1H), 7.97-7.84 (m, 1H), 7.71 (s, 2H), 7.55-7.46 (m, 1H), 7.33-7.27 (m, 2H), 7.22 (t, J = 7.9 Hz, 1H), 6.97 (d, J = 8.1 Hz, 1H), 5.14 (p, J = 6.9 Hz, 1H), 3.86 (t, J = 7.5 Hz, 2H), 3.80 (s, 2H), 3.67-3.58 (m, 5H), 2.87 (d, J = 4.8 Hz, 3H), 2.13-2.04 (m, 1H), 0.83 (dd, J = 6.4, 3.4 Hz, 4H). |
| 255 | 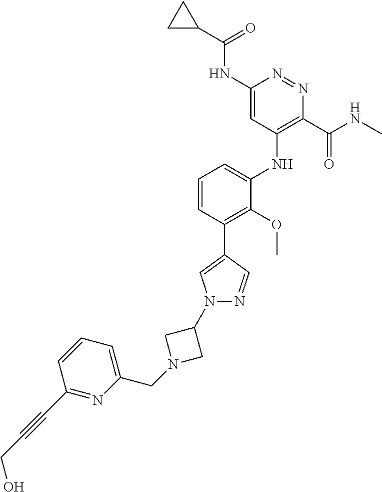 [SMILES: CNC(=O)c1nnc(NC(=O)C2CC2)cc1Nc1cccc(-c2cnn(C3CN(Cc4cccc(C#CCO)n4)C3)c2)c1OC] | 608.4 [M + H]+ | ¹H NMR (400 MHz, DMSO-d6) 11.33 (s, 1H), 10.99 (s, 1H), 9.18 (d, J = 4.9 Hz, 1H), 8.39 (s, 1H), 8.16 (s, 1H), 8.04 (s, 1H), 7.80 (t, J = 7.8 Hz, 1H), 7.49 (dd, J = 7.9, 1.5 Hz, 1H), 7.40 (dd, J = 11.3, 7.7 Hz, 2H), 7.31 (dd, J = 7.9, 1.6 Hz, 1H), 7.21 (t, J = 7.9 Hz, 1H), 5.44 (t, J = 6.0 Hz, 1H), 5.13 (p, J = 7.0 Hz, 1H), 4.33 (d, J = 6.0 Hz, 2H), 3.85-3.77 (m, 4H), 3.60 (s, 3H), 3.60 (t, J = 7.2 Hz, 2H), 2.87 (d, J = 4.8 Hz, 3H), 2.12-2.05 (m, 1H), 0.87- 0.80 (m, 4H). |
| 258 | 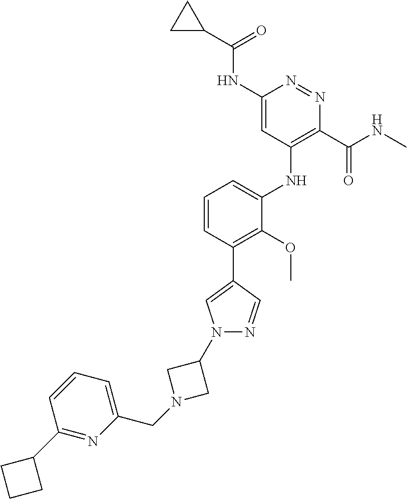 [SMILES: CNC(=O)c1nnc(NC(=O)C2CC2)cc1Nc1cccc(-c2cnn(C3CN(Cc4cccc(C5CCC5)n4)C3)c2)c1OC] | 608.4 [M + H]+ | ¹H NMR (400 MHz, DMSO-d6) 11.32 (s, 1H), 10.98 (s, 1H), 9.17 (d, J = 4.9 Hz, 1H), 8.38 (s, 1H), 8.16 (s, 1H), 8.03 (s, 1H), 7.67 (t, J = 7.7 Hz, 1H), 7.49 (dd, J = 7.8, 1.6 Hz, 1H), 7.30 (dd, J = 8.0, 1.6 Hz, 1H), 7.25-7.16 (m, 2H), 7.12 (d, J = 7.7 Hz, 1H), 5.12 (p, J = 7.0 Hz, 1H), 3.89-3.77 (m, 4H), 3.67- 3.53 (m, 6H), 2.87 (d, J = 4.8 Hz, 3H), 2.32-2.19 (m, 4H), 2.12-2.05 (m, 1H), 2.08-1.93 (m, 1H), 1.83 (ddt, J = 15.7, 7.8, 4.4 Hz, 1H), 0.86-0.79 (m, 4H). |
| 259 | 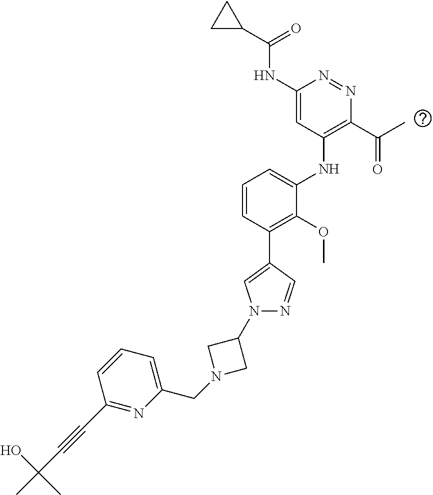 | 636.3 [M + H]+ | ¹H NMR (400 MHz, DMSO-d6) ¦Ä 11.33 (s, 1H), 10.99 (s, 1H), 9.18 (d, J = 5.0 Hz, 1H), 8.39 (s, 1H), 8.16 (s, 1H), 8.04 (s, 1H), 7.78 (t, J = 7.6 Hz, 1H), 7.52 [symbol] C 7.46 (m, 1H), 7.46 [symbol] C 7.25 (m, 3H), 7.21 (t, J = 7.8 Hz, 1H), 5.59 (s, 1H), 5.13 (s, 1H), 3.78 (d, J = 33.3 Hz, 4H), 3.60 (s, 4H), 2.87 (d, J = 4.8 Hz, 3H), 2.12 [symbol] C 1.95 (m, 1H), 1.48 (s, 6H), 1.24 (s, 1H), 0.83 (d, J = 8.1 Hz, 4H). |
| 260 | 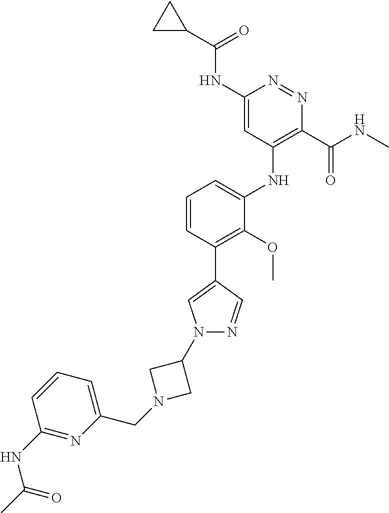 [SMILES: CNC(=O)c1nnc(NC(=O)C2CC2)cc1Nc1cccc(-c2cnn(C3CN(Cc4cccc(NC(C)=O)n4)C3)c2)c1OC] | 611.4 [M + H]+ | ¹H NMR (400 MHz, DMSO-d6) 11.33 (s, 1H), 10.99 (s, 1H), 10.47 (s, 1H), 9.18 (q, J = 4.7 Hz, 1H), 8.36 (s, 1H), 8.16 (s, 1H), 8.04 (s, 1H), 7.96 (d, J = 8.3 Hz, 1H), 7.74 (t, J = 7.9 Hz, 1H), 7.48 (dd, J = 7.9, 1.6 Hz, 1H), 7.31 (dd, J = 7.9, 1.6 Hz, 1H), 7.21 (t, J = 7.9 Hz, 1H), 7.11 (d, J = 7.4 Hz, 1H), 5.13 (p, J = 6.9 Hz, 1H), 3.82 (t, J = 7.4 Hz, 2H), 3.76 (s, 2H), 3.64-3.53 (m, 5H), 2.87 (d, J = 4.8 Hz, 3H), 2.12-2.04 (m, 4H), 0.88-0.78 (m, 4H). |
| 265 | 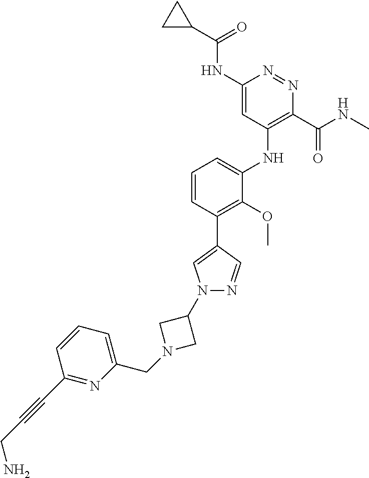 [SMILES: CNC(=O)c1nnc(NC(=O)C2CC2)cc1Nc1cccc(-c2cnn(C3CN(Cc4cccc(C#CCN)n4)C3)c2)c1OC] | 607.3 [M + H]+ | ¹H NMR (400 MHz, DMSO-d6) 11.32 (s, 1H), 10.98 (s, 1H), 9.18 (q, J = 4.8 Hz, 1H), 8.38 (s, 1H), 8.25 (s, 1H), 8.16 (s, 1H), 8.03 (s, 1H), 7.78 (t, J = 7.8 Hz, 1H), 7.48 (dd, J = 7.8, 1.6 Hz, 1H), 7.42-7.26 (m, 3H), 7.21 (t, J = 7.9 Hz, 1H), 5.12 (p, J = 6.9 Hz, 1H), 3.80 (t, J = 7.6 Hz, 5H), 3.63- 3.57 (m, 6H), 2.87 (d, J = 4.8 Hz, 3H), 2.08 (tt, J = 7.1, 5.3 Hz, 1H), 0.94-0.69 (m, 4H). |
| 267 | 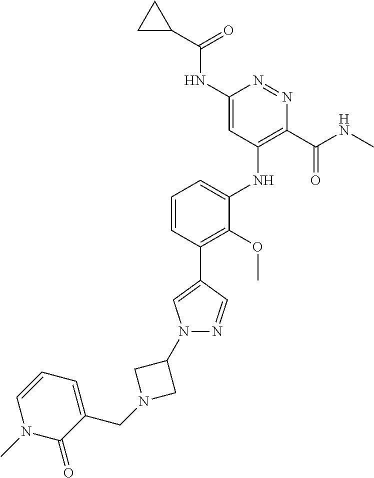 | 584.0 [M + H]+ | ¹H NMR (400 MHz, DMSO) δ 11.32 (s, 1H), 10.98 (s, 1H), 9.18 (d, J = 5.1 Hz, 1H), 8.40 (s, 1H), 8.17 (d, J = 19.2 Hz, 2H), 7.82 (dd, J = 45.0, 6.6 Hz, 2H), 7.48 (dd, J = 7.8, 1.6 Hz, 1H), 7.35-7.27 (m, 1H), 7.22 (t, J = 7.9 Hz, 1H), 6.36 (t, J = 6.8 Hz, 1H), 5.47- 5.35 (m, 1H), 4.60 (s, 2H), 4.53 (s, 2H), 4.40 (s, 2H), 3.60 (s, 3H), 3.52 (s, 3H), 2.86 (d, J = 4.8 Hz, 3H), 2.07 (dd, J = 8.6, 3.6 Hz, 1H), 0.90-0.78 (m, 4H) |
| 273 | 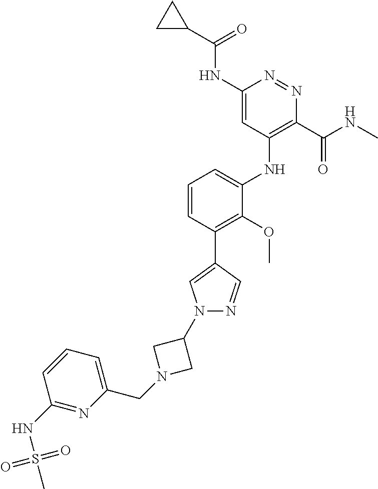 [SMILES: CNC(=O)c1nnc(NC(=O)C2CC2)cc1Nc1cccc(-c2cnn(C3CN(Cc4cccc(NS(C)(=O)=O)n4)C3)c2)c1OC] | 646.2 [M + H]+ | ¹H NMR (400 MHz, DMSO-d6) 11.32 (s, 1H), 10.98 (s, 1H), 9.17 (q, J = 4.7 Hz, 1H), 8.38 (s, 1H), 8.15 (d, J = 1.2 Hz, 1H), 8.03 (s, 1H), 7.71 (t, J = 7.8 Hz, 1H), 7.48 (dd, J = 7.8, 1.6 Hz, 1H), 7.30 (dd, J = 7.9, 1.6 Hz, 1H), 7.21 (t, J = 7.9 Hz, 1H), 7.02 (d, J = 7.4 Hz, 1H), 6.87 (d, J = 8.2 Hz, 1H), 5.12 (p, J = 6.9 Hz, 1H), 3.85 (t, J = 7.5 Hz, 2H), 3.77 (s, 2H), 3.65-3.59 (m, 5H), 3.32 (s, 3H), 2.87 (d, J = 4.8 Hz, 3H), 2.08 (tt, J = 7.0, 5.3 Hz, 1H), 0.88-0.79 (m, 4H) |
| 274 | 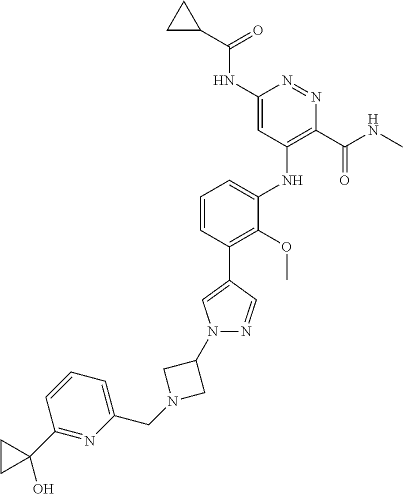 [SMILES: CNC(=O)c1nnc(NC(=O)C2CC2)cc1Nc1cccc(-c2cnn(C3CN(Cc4cccc(C5(O)CC5)n4)C3)c2)c1OC] | 610.4 [M + H]+ | ¹H NMR (400 MHz, DMSO-d6) 11.32 (s, 1H), 10.98 (s, 1H), 9.17 (d, J = 4.9 Hz, 1H), 8.37 (s, 1H), 8.16 (s, 1H), 8.02 (s, 1H), 7.72 (t, J = 7.7 Hz, 1H), 7.58 (dd, J = 7.9, 1.0 Hz, 1H), 7.48 (dd, J = 7.8, 1.6 Hz, 1H), 7.30 (dd, J = 7.9, 1.6 Hz, 1H), 7.20 (t, J = 7.9 Hz, 1H), 7.14 (dd, J = 7.6, 1.1 Hz, 1H), 6.08 (s, 1H), 5.10 (p, J = 6.9 Hz, 1H), 3.79 (t, J = 7.3 Hz, 2H), 3.73 (s, 2H), 3.63-3.51 (m, 5H), 2.87 (d, J = 4.8 Hz, 3H), 2.15-2.03 (m, 1H), 1.22 (dt, J = 6.8, 3.5 Hz, 2H), 1.06 (q, J = 3.9 Hz, 2H), 0.87-0.77 (m, 4H). |
| 275 | 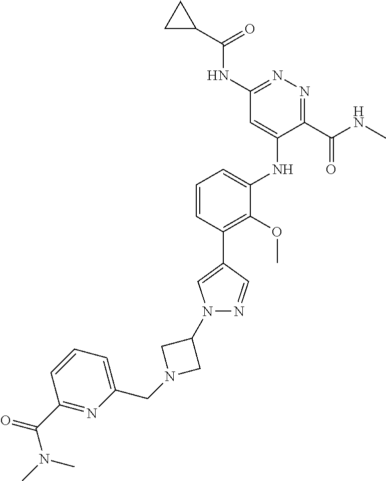 [SMILES: CNC(=O)c1nnc(NC(=O)C2CC2)cc1Nc1cccc(-c2cnn(C3CN(Cc4cccc(C(=O)N(C)C)n4)C3)c2)c1OC] | 625.3 | ¹H NMR (400 MHz, DMSO-d6) 11.32 (s, 1H), 10.98 (s, 1H), 9.17 (q, J = 4.7 Hz, 1H), 8.38 (s, 1H), 8.16 (s, 1H), 8.03 (s, 1H), 7.90 (t, J = 7.7 Hz, 1H), 7.48 (dd, J = 7.8, 1.7 Hz, 2H), 7.42 (dd, J = 7.7, 1.0 Hz, 1H), 7.30 (dd, J = 8.0, 1.6 Hz, 1H), 7.21 (t, J = 7.9 Hz, 1H), 5.14 (p, J = 6.9 Hz, 1H), 3.91-3.80 (m, 4H), 3.59 (s, 5H), 3.01 (s, 3H), 2.94 (s, 3H), 2.87 (d, J = 4.8 Hz, 3H), 2.14-2.03 (m, 1H), 0.88-0.77 (m, 4H). |
| 276 | 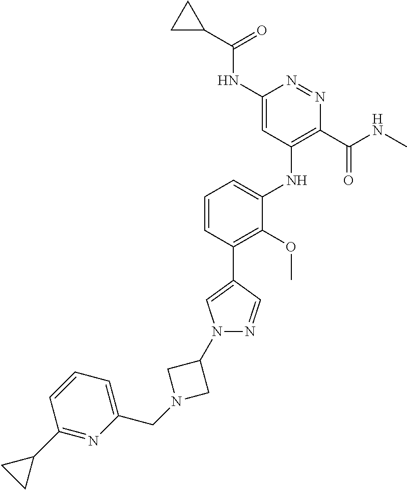 [SMILES: CNC(=O)c1nnc(NC(=O)C2CC2)cc1Nc1cccc(-c2cnn(C3CN(Cc4cccc(C5CC5)n4)C3)c2)c1OC] | 594.3 [M + H]+ | ¹H NMR (400 MHz, DMSO-d6) 11.32 (s, 1H), 10.98 (s, 1H), 9.18 (q, J = 4.8 Hz, 1H), 8.37 (s, 1H), 8.16 (s, 1H), 8.03 (s, 1H), 7.60 (t, J = 7.7 Hz, 1H), 7.48 (dd, J = 7.8, 1.6 Hz, 1H), 7.30 (dd, J = 7.9, 1.6 Hz, 1H), 7.20 (t, J = 7.9 Hz, 1H), 7.13 (ddd, J = 8.8, 7.6, 1.0 Hz, 2H), 5.11 (t, J = 7.0 Hz, 1H), 3.79 (td, J = 7.1, 1.6 Hz, 2H), 3.73 (s, 2H), 3.58 (d, J = 11.0 Hz, 5H), 2.87 (d, J = 4.8 Hz, 3H), 2.07 (dtd, J = 10.9, 5.3, 4.8, 2.6 Hz, 2H), 0.96-0.88 (m, 4H), 0.87- 0.76 (m, 4H). |
| 277 | 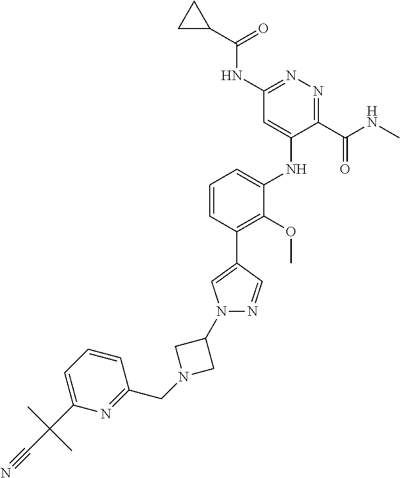 [SMILES: CNC(=O)c1nnc(NC(=O)C2CC2)cc1Nc1cccc(-c2cnn(C3CN(Cc4cccc(C(C)(C)C#N)n4)C3)c2)c1OC] | 621.4 [M + H]+ | ¹H NMR (400 MHz, DMSO-d6) 11.33 (s, 1H), 10.99 (s, 1H), 9.19 (d, J = 4.9 Hz, 1H), 8.39 (s, 1H), 8.16 (s, 1H), 8.04 (s, 1H), 7.87 (t, J = 7.8 Hz, 1H), 7.49 (dd, J = 7.8, 1.4 Hz, 2H), 7.39 (d, J = 7.7 Hz, 1H), 7.31 (dd, J = 7.9, 1.6 Hz, 1H), 7.21 (t, J = 7.9 Hz, 1H), 5.14 (p, J = 7.0 Hz, 1H), 3.89-3.81 (m, 4H), 3.62 (d, J = 15.8 Hz, 5H), 2.87 (d, J = 4.8 Hz, 3H), 2.12-2.05 (m, 1H), 1.71 (s, 6H), 0.86- 0.80 (m, 4H). |
| 281 | 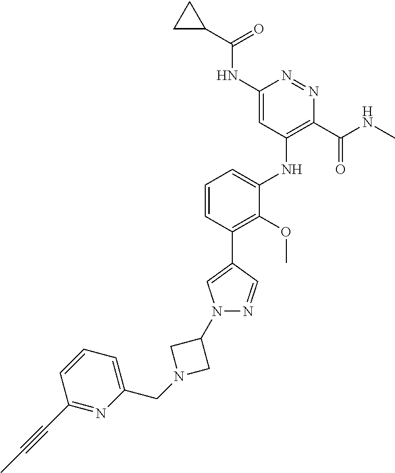 [SMILES: CC#Cc1cccc(CN2CC(n3cc(-c4cccc(Nc5cc(NC(=O)C6CC6)nnc5C(=O)NC)c4OC)cn3)C2)n1] | 592.2 [M + H]+ | ¹H NMR (400 MHz, DMSO-d6) 11.32 (s, 1H), 10.98 (s, 1H), 9.18 (q, J = 4.7 Hz, 1H), 8.38 (s, 1H), 8.15 (s, 1H), 8.03 (s, 1H), 7.75 (t, J = 7.7 Hz, 1H), 7.48 (dd, J = 7.8, 1.6 Hz, 1H), 7.39-7.27 (m, 3H), 7.20 (t, J = 7.9 Hz, 1H), 5.16-5.08 (m, 1H), 3.80 (s, 4H), 3.59 (s, 4H), 2.87 (d, J = 4.8 Hz, 3H), 2.08-2.07 (m, 4H), 0.86-0.79 (m, 4H). |
| 287 | 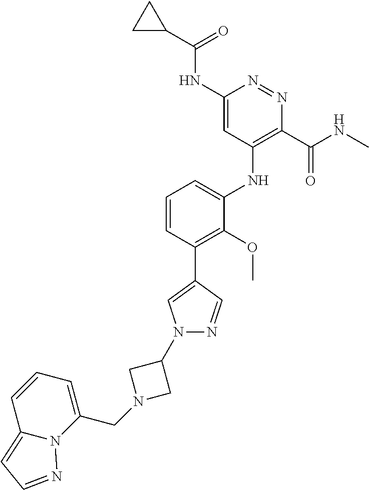 [SMILES: CNC(=O)c1nnc(NC(=O)C2CC2)cc1Nc1cccc(-c2cnn(C3CN(Cc4cccc5ccnn45)C3)c2)c1OC] | 593.2 [M + H]+ | ¹H NMR (500 MHz, DMSO) 8 11.29 (s, 1H), 10.96 (s, 1H), 9.14 (q, J = 4.7 Hz, 1H), 8.42 (d, J = 0.8 Hz, 1H), 8.14 (s, 1H), 8.05- 7.99 (m, 2H), 7.65 (dd, J = 8.9, 1.3 Hz, 1H), 7.47 (dd, J = 7.8, 1.5 Hz, 1H), 7.31- 7.17 (m, 3H), 6.91 (dd, J = 6.9, 1.4 Hz, 1H), 6.66 (d, J = 2.3 Hz, 1H), 5.18 (p, J = 6.9 Hz, 1H), 4.23 (s, 2H), 3.95 (t, J = 7.4 Hz, 2H), 3.71 (t, J = 7.1 Hz, 2H), 3.59 (s, 3H), 2.86 (d, J = 4.8 Hz, 3H), 2.07 (p, J = 6.6 Hz, 1H), 0.86-0.72 (m, 4H). |
| 290 | 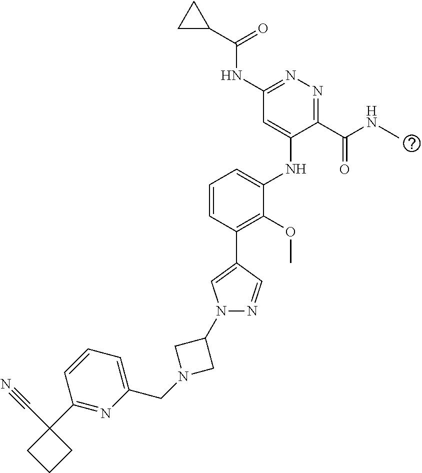 [SMILES: CNC(=O)c1nnc(NC(=O)C2CC2)cc1Nc1cccc(-c2cnn(C3CN(Cc4cccc(C5(C#N)CCC5)n4)C3)c2)c1OC] | 633.3 [M + H]+ | ¹H NMR (400 MHz, DMSO-d6) 11.32 (s, 1H), 10.99 (s, 1H), 9.17 (q, J = 4.7 Hz, 1H), 8.39 (s, 1H), 8.16 (d, J = 5.0 Hz, 1H), 8.03 (s, 1H), 7.87 (t, J = 7.8 Hz, 1H), 7.54-7.45 (m, 2H), 7.45-7.37 (m, 1H), 7.30 (dd, J = 7.9, 1.6 Hz, 1H), 7.20 (t, J = 7.9 Hz, 1H), 5.14 (p, J = 6.9 Hz, 1H), 3.95-3.81 (m, 4H), 3.64 (td, J = 6.7, 1.6 Hz, 2H), 3.59 (s, 3H), 2.87 (d, J = 4.8 Hz, 3H), 2.85- 2.63 (m, 4H), 2.26 (dp, J = 11.3, 8.6 Hz, 1H), 2.15-1.98 (m, 2H), 0.91-0.67 (m, 4H). |
| 292 | 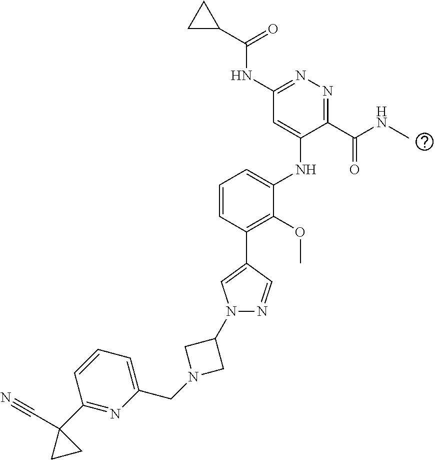 [SMILES: CNC(=O)c1nnc(NC(=O)C2CC2)cc1Nc1cccc(-c2cnn(C3CN(Cc4cccc(C5(C#N)CC5)n4)C3)c2)c1OC] | 619.3 [M + H]+ | ¹H NMR (400 MHz, DMSO-d6) 11.32 (s, 1H), 10.99 (s, 1H), 9.17 (q, J = 4.8 Hz, 1H), 8.37 (s, 1H), 8.16 (s, 1H), 8.03 (s, 1H), 7.81 (t, J = 7.8 Hz, 1H), 7.51-7.40 (m, 2H), 7.35- 7.28 (m, 2H), 7.20 (t, J = 7.9 Hz, 1H), 5.12 (p, J = 7.0 Hz, 1H), 3.88-3.75 (m, 4H), 3.59 (s, 5H), 2.87 (d, J = 4.8 Hz, 3H), 2.14-2.03 (m, 1H), 1.81-1.71 (m, 2H), 1.69 (q, J = 5.2, 4.5 Hz, 2H), 0.88- 0.77 (m, 4H). |
| 294 | 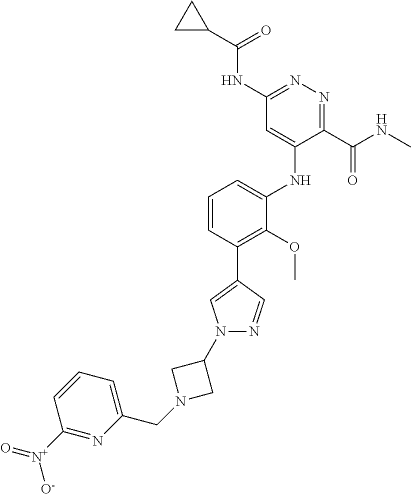 [SMILES: CNC(=O)c1nnc(NC(=O)C2CC2)cc1Nc1cccc(-c2cnn(C3CN(Cc4cccc([N+](=O)[O-])n4)C3)c2)c1OC] | 599.3 [M + H]+ | ¹H NMR (400 MHz, DMSO-d6) 11.31 (s, 1H), 10.98 (s, 1H), 9.17 (d, J = 4.9 Hz, 1H), 8.41 (s, 1H), 8.29-8.13 (m, 2H), 8.05 (s, 1H), 7.92 (dd, J = 7.0, 1.6 Hz, 1H), 7.49 (dd, J = 7.9, 1.6 Hz, 1H), 7.31 (dd, J = 7.9, 1.6 Hz, 1H), 7.21 (m, 2H), 5.25-5.09 (m, 1H), 4.16-3.68 (m, 9H), 2.87 (d, J = 4.8 Hz, 3H), 2.13-2.00 (m, 1H), 0.88-0.70 (m, 4H). |
| 295 | 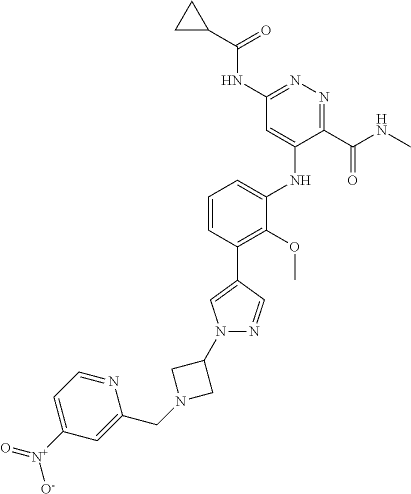 [SMILES: CNC(=O)c1nnc(NC(=O)C2CC2)cc1Nc1cccc(-c2cnn(C3CN(Cc4cc([N+](=O)[O-])ccn4)C3)c2)c1OC] | 599.2 [M + H]+ | ¹H NMR (Chloroform-d, 400 MHz) 11.15 (1H, s), 9.02 (1H, s), 8.91 (1H, dd, J = 5.4, 0.6 Hz), 8.29 (1H, s), 8.17 (2H, dd, J = 7.5, 3.7 Hz), 8.05 (1H, d, J = 0.7 Hz), 8.00 (1H, s), 7.96 (1H, dd, J = 5.4, 2.2 Hz), 7.38 (1H, dd, J = 7.8, 1.6 Hz), 7.32 (1H, dd, J = 7.9, 1.6 Hz), 7.22 (1H, t, J = 7.9 Hz), 5.15 (1H, p, J = 7.0 Hz), 4.18 (4H, d, J = 21.0 Hz), 3.90 (2H, s), 3.68 (3H, s), 3.08 (3H, d, J = 5.1 Hz), 1.74 (1H, tt, J = 8.1, 4.5 Hz), 1.18- 1.09 (2H, m), 0.95 (2H, dq, J = 7.6, 4.1 Hz). |
| 299 | 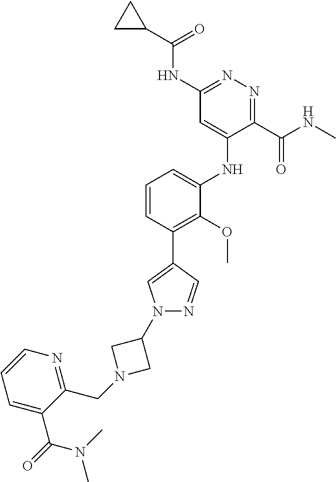 [SMILES: CNC(=O)c1nnc(NC(=O)C2CC2)cc1Nc1cccc(-c2cnn(C3CN(Cc4ncccc4C(=O)N(C)C)C3)c2)c1OC] | 625.4 [M + H]+ | ¹H NMR (400 MHz, DMSO-d6) 11.32 (s, 1H), 10.98 (s, 1H), 9.17 (q, J = 4.8 Hz, 1H), 8.53 (dd, J = 4.9, 1.7 Hz, 1H), 8.36 (s, 1H), 8.15 (s, 1H), 8.02 (s, 1H), 7.64 (dd, J = 7.6, 1.7 Hz, 1H), 7.48 (dd, J = 7.8, 1.6 Hz, 1H), 7.39-7.27 (m, 2H), 7.20 (t, J = 7.9 Hz, 1H), 5.05 (p, J = 6.7 Hz, 1H), 3.88-3.66 (m, 4H), 3.59 (d, J = 3.0 Hz, 5H), 3.02 (s, 3H), 2.87 (d, J = 4.8 Hz, 3H), 2.79 (s, 3H), 2.14- 1.99 (m, 1H), 0.92-0.68 (m, 4H). |
| 300 | 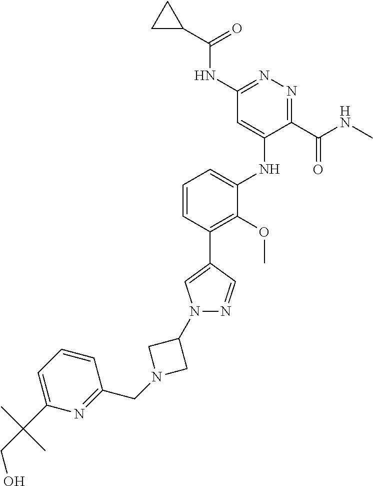 [SMILES: CNC(=O)c1nnc(NC(=O)C2CC2)cc1Nc1cccc(-c2cnn(C3CN(Cc4cccc(C(C)(C)CO)n4)C3)c2)c1OC] | 626.4 [M + H]+ | ¹H NMR (400 MHz, DMSO-d6) 11.31 (s, 1H), 10.98 (s, 1H), 9.17 (q, J = 4.8 Hz, 1H), 8.37 (s, 1H), 8.17 (d, J = 10.7 Hz, 1H), 8.03 (s, 1H), 7.69 (t, J = 7.8 Hz, 1H), 7.48 (dd, J = 7.8, 1.6 Hz, 1H), 7.35-7.11 (m, 4H), 5.12 (p, J = 7.0 Hz, 1H), 4.68 (s, 1H), 4.01- 3.71 (m, 4H), 3.67-3.43 (m, 7H), 2.87 (d, J = 4.8 Hz, 3H), 2.12-2.03 (m, 1H), 1.24 (s, 6H), 0.97-0.70 (m, 4H). |
| 302 | 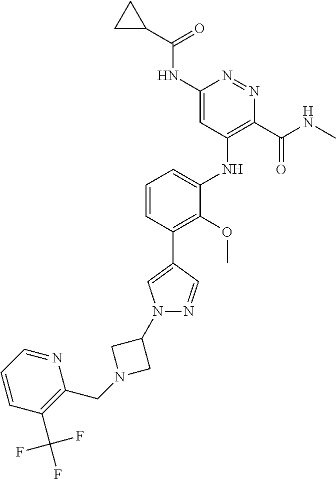 [SMILES: CNC(=O)c1nnc(NC(=O)C2CC2)cc1Nc1cccc(-c2cnn(C3CN(Cc4ncccc4C(F)(F)F)C3)c2)c1OC] | 622.3 [M + H]+ | ¹H NMR (500 MHz, DMSO) & 11.28 (s, 1H), 10.95 (s, 1H), 9.14 (q, J = 4.7 Hz, 1H), 8.80 (dd, J = 4.7, 1.6 Hz, 1H), 8.33 (s, 1H), 8.16 (dd, J = 8.0, 1.7 Hz, 1H), 8.13 (s, 1H), 8.01 (s, 1H), 7.54 (dd, J = 8.0, 4.8 Hz, 1H), 7.46 (dd, J = 7.8, 1.6 Hz, 1H), 7.28 (dd, J = 7.9, 1.6 Hz, 1H), 7.19 (t, J = 7.9 Hz, 1H), 5.07 (p, J = 6.9 Hz, 1H), 3.96 (s, 2H), 3.82 (td, J = 7.0, 1.7 Hz, 2H), 3.68 (dd, J = 7.9, 6.4 Hz, 2H), 3.57 (s, 3H), 2.85 (d, J = 4.7 Hz, 3H), 2.07 (tt, J = 7.0, 5.3 Hz, 1H), 0.92-0.72 (m, 4H). |
| 303 | 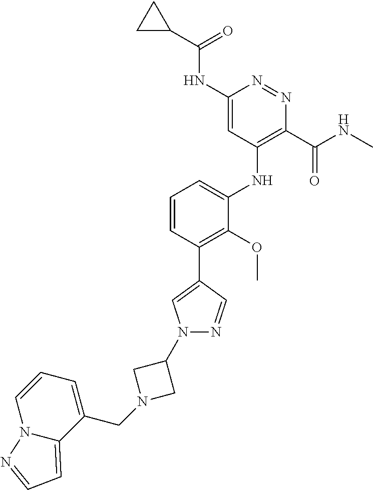 [SMILES: CNC(=O)c1nnc(NC(=O)C2CC2)cc1Nc1cccc(-c2cnn(C3CN(Cc4cccn5nccc45)C3)c2)c1OC] | 593.2 [M + H]+ | ¹H NMR (400 MHz, DMSO) δ 11.30 (s, 1H), 10.97 (s, 1H), 9.20-9.12 (m, 1H), 8.57 (dt, J = 6.9, 1.0 Hz, 1H), 8.37 (d, J = 0.8 Hz, 1H), 8.14 (s, 1H), 8.02 (d, J = 0.7 Hz, 1H), 7.98 (d, J = 2.2 Hz, 1H), 7.46 (dd, J = 7.8, 1.6 Hz, 1H), 7.29 (dd, J = 7.9, 1.6 Hz, 1H), 7.20 (d, J = 7.9 Hz, 1H), 7.18-7.14 (m, 1H), 6.85 (t, J = 6.9 Hz, 1H), 6.71 (dd, J = 2.3, 0.9 Hz, 1H), 5.12 (p, J = 6.9 Hz, 1H), 3.91 (s, 2H), 3.80 (td, J = 7.1, 1.6 Hz, 2H), 3.60- 3.54 (m, 5H), 2.86 (d, J = 4.8 Hz, 3H), 2.11-2.03 (m, 1H), 0.83-0.77 (m, 4H). |
| 304 | 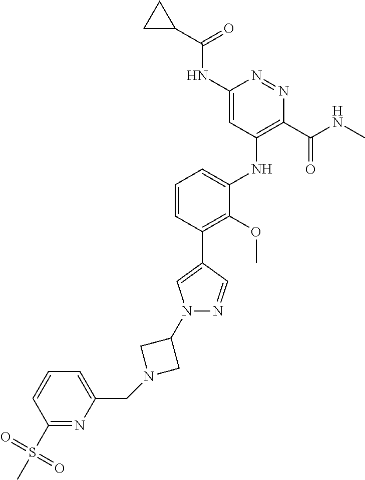 [SMILES: CNC(=O)c1nnc(NC(=O)C2CC2)cc1Nc1cccc(-c2cnn(C3CN(Cc4cccc(S(C)(=O)=O)n4)C3)c2)c1OC] | 632.4 [M + H]+ | ¹H NMR (500 MHz, DMSO) δ 11.30 (s, 1H), 10.97 (s, 1H), 9.15 (q, J = 4.8 Hz, 1H), 8.39 (s, 1H), 8.21-8.08 (m, 2H), 8.03 (s, 1H), 7.94 (dd, J = 7.8, 1.0 Hz, 1H), 7.76 (dd, J = 7.9, 0.9 Hz, 1H), 7.47 (dd, J = 7.9, 1.6 Hz, 1H), 7.30 (dd, J = 7.9, 1.5 Hz, 1H), 7.20 (t, J = 7.9 Hz, 1H), 5.14 (p, J = 6.9 Hz, 1H), 3.96 (s, 2H), 3.86 (td, J = 7.1, 1.7 Hz, 2H), 3.66 (td, J = 6.7, 1.6 Hz, 2H), 3.59 (s, 3H), 3.28 (s, 3H), 2.86 (d, J = 4.8 Hz, 3H), 2.08 (tt, J = 7.0, 5.2 Hz, 1H), 0.87-0.67 (m, 4H). |
| 306 | 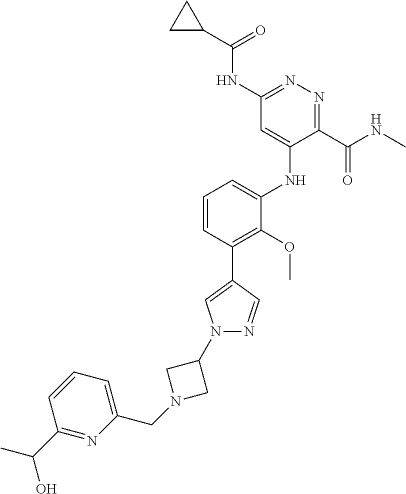 [SMILES: CNC(=O)c1nnc(NC(=O)C2CC2)cc1Nc1cccc(-c2cnn(C3CN(Cc4cccc(C(C)O)n4)C3)c2)c1OC] | | ¹H NMR (500 MHz, DMSO) δ 11.29 (s, 1H), 10.96 (s, 1H), 9.14 (q, J = 4.8 Hz, 1H), 8.36 (s, 1H), 8.13 (s, 1H), 8.01 (s, 1H), 7.74 (t, J = 7.7 Hz, 1H), 7.46 (dd, J = 7.8, 1.5 Hz, 1H), 7.37 (d, J = 7.7 Hz, 1H), 7.29 (dd, J = 7.9, 1.6 Hz, 1H), 7.26-7.16 (m, 2H), 5.28 (d, J = 4.6 Hz, 1H), 5.11 (p, J = 6.9 Hz, 1H), 4.73-4.64 (m, 1H), 3.81 (s, 4H), 3.58 (s, 3H), 2.85 (d, J = 4.7 Hz, 3H), 2.07 (ddd, J = 12.5, 7.3, 5.4 Hz, 1H), 1.34 (d, J = 6.5 Hz, 3H), 0.80 (h, J = 3.3 Hz, 4H) |
| 307 | 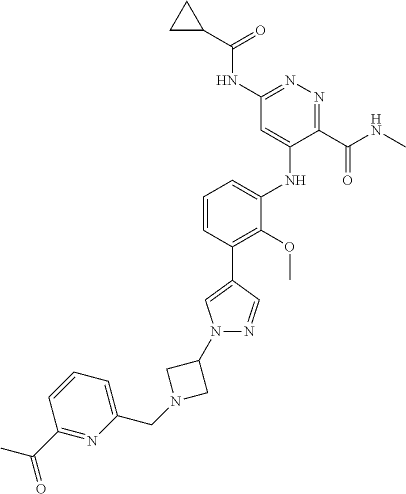 [SMILES: CNC(=O)c1nnc(NC(=O)C2CC2)cc1Nc1cccc(-c2cnn(C3CN(Cc4cccc(C(C)=O)n4)C3)c2)c1OC] | | ¹H NMR (500 MHz, DMSO) 8 11.29 (s, 1H), 10.96 (s, 1H), 9.14 (q, J = 4.8 Hz, 1H), 8.38 (s, 1H), 8.13 (s, 1H), 8.07-7.91 (m, 4H), 7.86-7.77 (m, 2H), 7.77-7.63 (m, 2H), 7.46 (ddd, J = 7.5, 3.6, 1.4 Hz, 1H), 7.29 (dd, J = 7.9, 1.6 Hz, 1H), 7.19 (t, J = 7.9 Hz, 1H), 5.14 (p, J = 6.9 Hz, 1H), 3.93 (s, 2H), 3.87 (td, J = 7.0, 1.7 Hz, 2H), 3.65 (td, J = 6.7, 1.7 Hz, 2H), 3.58 (s, 3H), 2.85 (d, J = 4.7 Hz, 3H), 2.63 (s, 3H), 2.07 (tt, J = 7.0, 5.3 Hz, 1H), 0.81 (dt, J = 7.8, 2.2 Hz, 4H). |
| 308 | 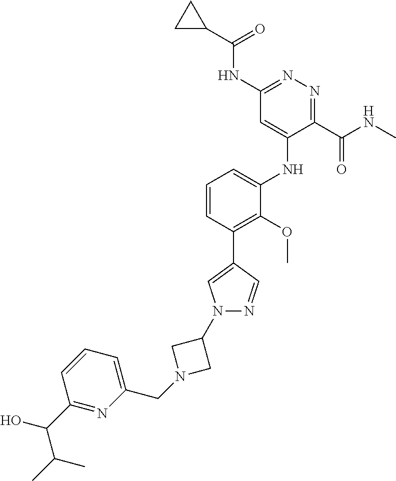 [SMILES: CNC(=O)c1nnc(NC(=O)C2CC2)cc1Nc1cccc(-c2cnn(C3CN(Cc4cccc(C(O)C(C)C)n4)C3)c2)c1OC] | | ¹H NMR (500 MHz, DMSO) δ 11.29 (s, 1H), 10.95 (s, 1H), 9.14 (q, J = 4.8 Hz, 1H), 8.36 (d, J = 11.2 Hz, 1H), 8.13 (s, 1H), 8.00 (s, 1H), 7.73 (t, J = 7.7 Hz, 1H), 7.46 (dd, J = 7.9, 1.5 Hz, 1H), 7.33-7.26 (m, 2H), 7.25-7.15 (m, 2H), 6.64 (s, 1H), 5.16 (d, J = 5.0 Hz, 1H), 5.09 (p, J = 7.0 Hz, 1H), 4.32 (t, J = 4.4 Hz, 1H), 3.78 (d, J = 11.2 Hz, 4H), 3.58 (s, 4H), 3.61-3.53 (m, 1H), 2.85 (d, J = 4.8 Hz, 3H), 2.11-1.96 (m, 2H), 0.86-0.77 (m, 7H), 0.74 (d, J = 6.8 Hz, 3H) |
| 309 | 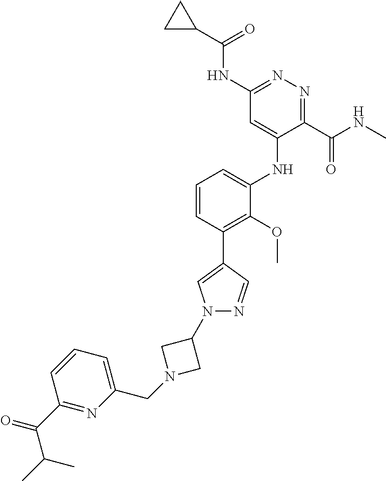 [SMILES: CNC(=O)c1nnc(NC(=O)C2CC2)cc1Nc1cccc(-c2cnn(C3CN(Cc4cccc(C(=O)C(C)C)n4)C3)c2)c1OC] | | ¹H NMR (500 MHz, DMSO) δ 11.29 (s, 1H), 10.96 (s, 1H), 9.14 (d, J = 4.9 Hz, 1H), 8.37 (d, J = 0.8 Hz, 1H), 8.13 (s, 1H), 8.02 (s, 1H), 8.03-7.94 (m, 4H), 7.97-7.92 (m, 1H), 7.84 (ddd, J = 8.1, 2.8, 1.0 Hz, 2H), 7.77-7.62 (m, 3H), 7.50-7.43 (m, 1H), 7.29 (dd, J = 8.0, 1.5 Hz, 1H), 7.24-7.16 (m, 1H), 5.14 (t, J = 7.0 Hz, 1H), 4.17 (s, 1H), 4.04 (h, J = 6.8 Hz, 1H), 3.94 (s, 2H), 3.87 (s, 2H), 3.66 (s, 2H), 3.58 (s, 3H), 2.85 (d, J = 4.7 Hz, 3H), 2.07 (dd, J = 6.6, 4.3 Hz, 1H), 1.11 (d, J = 6.9 Hz, 5H), 0.81 (dt, J = 8.1, 2.4 Hz, 4H). |
| 313 | 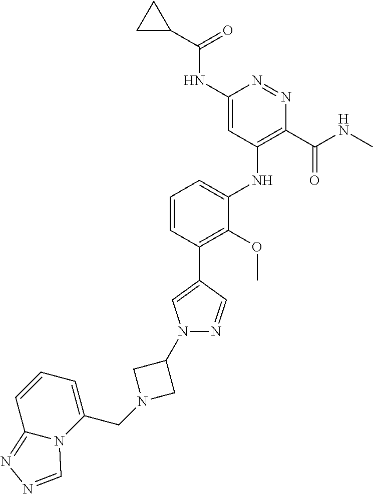 | 591.4 [M + H]+ | ¹H NMR (500 MHz, DMSO) δ 11.29 (s, 1H), 10.96 (s, 1H), 9.35 (d, J = 0.8 Hz, 1H), 9.15 (q, J = 4.8 Hz, 1H), 8.36 (s, 1H), 8.13 (s, 1H), 8.03 (s, 1H), 7.72 (d, J = 9.2 Hz, 1H), 7.46 (dd, J = 7.8, 1.5 Hz, 1H), 7.36 (dd, J = 9.2, 6.6 Hz, 1H), 7.29 (dd, J = 8.0, 1.5 Hz, 1H), 7.19 (t, J = 7.9 Hz, 1H), 6.98 (d, J = 6.7 Hz, 1H), 5.13 (p, J = 7.0 Hz, 1H), 4.11 (s, 2H), 3.83 (t, J = 7.8 Hz, 2H), 3.65 (t, J = 7.5 Hz, 2H), 3.58 (s, 3H), 2.86 (d, J = 4.8 Hz, 3H), 2.10-2.03 (m, 1H), 0.83-0.76 (m, 4H). |
| 324 | 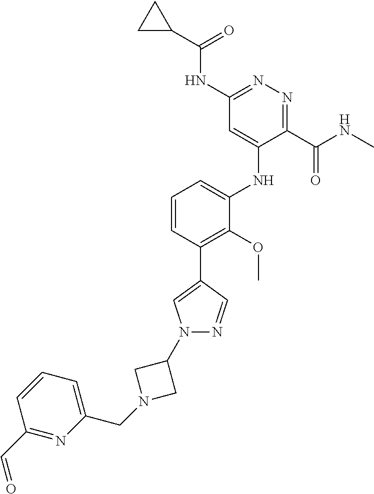 [SMILES: CNC(=O)c1nnc(NC(=O)C2CC2)cc1Nc1cccc(-c2cnn(C3CN(Cc4cccc(C=O)n4)C3)c2)c1OC] | | ¹H NMR (500 MHz, DMSO) δ 11.29 (s, 1H), 10.96 (s, 1H), 9.96 (d, J = 0.7 Hz, 1H), 9.14 (q, J = 4.8 Hz, 1H), 8.38 (d, J = 0.7 Hz, 1H), 8.13 (s, 1H), 8.06- 7.99 (m, 2H), 7.82 (dd, J = 7.6, 1.1 Hz, 1H), 7.72 (dd, J = 7.8, 1.1 Hz, 1H), 7.47 (dd, J = 7.8, 1.5 Hz, 1H), 7.29 (dd, J = 7.9, 1.6 Hz, 1H), 7.19 (t, J = 7.9 Hz, 1H), 5.13 (p, J = 6.9 Hz, 1H), 3.96 (s, 2H), 3.84 (td, J = 7.1, 1.6 Hz, 2H), 3.64 (dd, J = 8.0, 6.4 Hz, 2H), 3.58 (s, 3H), 2.85 (d, J = 4.9 Hz, 3H), 2.11-2.02 (m, 1H), 0.81 (dt, J = 7.7, 2.3 Hz, 4H), 0.79 (s, 1H). |
| 343 | 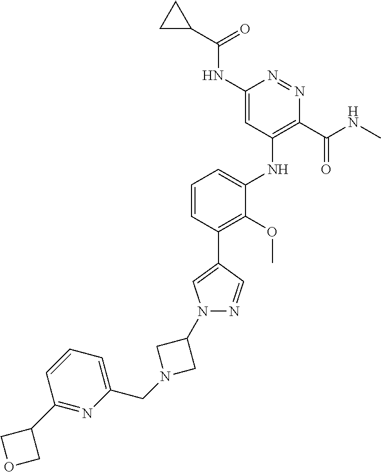 [SMILES: CNC(=O)c1nnc(NC(=O)C2CC2)cc1Nc1cccc(-c2cnn(C3CN(Cc4cccc(C5COC5)n4)C3)c2)c1OC] | 610.3 [M + H]+ | ¹H NMR (400 MHz, DMSO-d6) 11.32 (s, 1H), 10.98 (s, 1H), 9.17 (d, J = 5.0 Hz, 1H), 8.39 (s, 1H), 8.16 (s, 1H), 8.03 (s, 1H), 7.75 (t, J = 7.7 Hz, 1H), 7.48 (dd, J = 7.9, 1.6 Hz, 1H), 7.30 (dd, J = 7.8, 2.0 Hz, 2H), 7.25-7.16 (m, 2H), 5.13 (p, J = 7.0 Hz, 1H), 4.88 (dd, J = 8.5, 5.5 Hz, 2H), 4.79 (dd, J = 6.9, 5.6 Hz, 2H), 4.38 (tt, J = 8.4, 6.8 Hz, 1H), 3.89-3.80 (m, 4H), 3.62 (dd, J = 7.9, 6.4 Hz, 2H), 3.59 (s, 3H), 2.87 (d, J = 4.7 Hz, 3H), 2.14-2.03 (m, 1H), 0.86-0.79 (m, 4H) |
| 344 | 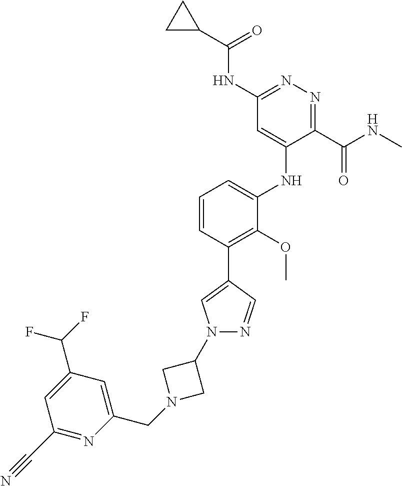 [SMILES: CNC(=O)c1nnc(NC(=O)C2CC2)cc1Nc1cccc(-c2cnn(C3CN(Cc4cc(C(F)F)cc(C#N)n4)C3)c2)c1OC] | 629.4 [M + H]+ | ¹H NMR (400 MHz, Chloroform-d) 11.16 (s, 1H), 8.95 (s, 1H), 8.29 (s, 1H), 8.16 (d, J = 5.8 Hz, 1H), 8.03 (d, J = 27.5 Hz, 2H), 7.79 (d, J = 28.2 Hz, 2H), 7.42-7.30 (m, 2H), 7.22 (t, J = 7.8 Hz, 1H), 6.72 (t, J = 55.2 Hz, 1H), 5.11 (p, J = 6.8 Hz, 1H), 4.11 (s, 2H), 3.96 (dt, J = 80.8, 7.5 Hz, 4H), 3.67 (s, 3H), 3.08 (d, J = 5.0 Hz, 3H), 1.73 (tt, J = 8.1, 4.3 Hz, 1H), 1.14 (p, J = 4.0 Hz, 2H), 0.95 (dq, J = 7.5, 3.9 Hz, 2H). |
| 351 | 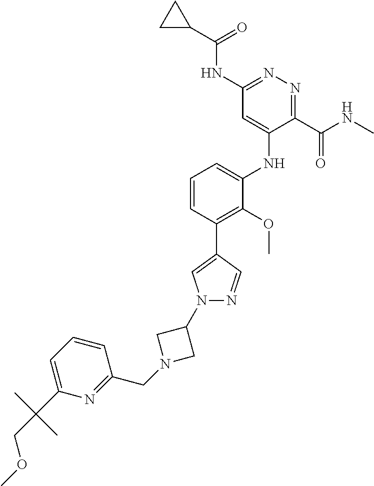 | 640.4 [M + H]+ | ¹H NMR (400 MHz, DMSO-d6) 11.31 (s, 1H), 10.98 (s, 1H), 9.17 (d, J = 5.1 Hz, 1H), 8.37 (s, 1H), 8.15 (s, 1H), 8.03 (s, 1H), 7.68 (t, J = 7.8 Hz, 1H), 7.48 (dd, J = 7.8, 1.7 Hz, 1H), 7.36-7.11 (m, 4H), 5.11 (q, J = 7.0 Hz, 1H), 3.81 (d, J = 8.1 Hz, 4H), 3.60 (d, J = 7.8 Hz, 5H), 3.50 (s, 2H), 3.18 (s, 3H), 2.14-2.01 (m, 1H), 1.27 (s, 6H), 0.83 (dd, J = 7.2, 3.6 Hz, 4H). |
| 365 | 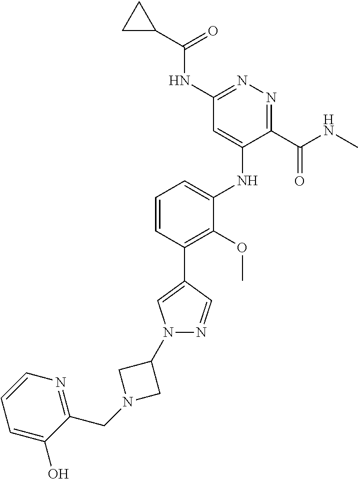 [SMILES: CNC(=O)c1nnc(NC(=O)C2CC2)cc1Nc1cccc(-c2cnn(C3CN(Cc4ncccc4O)C3)c2)c1OC] | 570.3 [M + H]+ | ¹H NMR (400 MHz, DMSO-d6) 11.36 (d, J = 5.0 Hz, 1H), 11.01 (d, J = 3.5 Hz, 1H), 10.66 (s, 2H), 9.19 (q, J = 4.8 Hz, 1H), 8.44 (s, 1H), 8.20 (s, 1H), 8.15 (s, 1H), 8.10 (dd, J = 3.8, 2.2 Hz, 1H), 7.50 (dd, J = 7.8, 1.6 Hz, 1H), 7.37- 7.28 (m, 3H), 7.23 (t, J = 7.9 Hz, 1H), 5.53 (q, J = 7.5 Hz, 1H), 4.75 (s, 4H), 4.64-4.54 (m, 2H), 3.62 (s, 3H), 2.87 (d, J = 4.8 Hz, 3H), 2.16- 2.01 (m, 1H), 0.88-0.70 (m, 4H). |
| 369 | 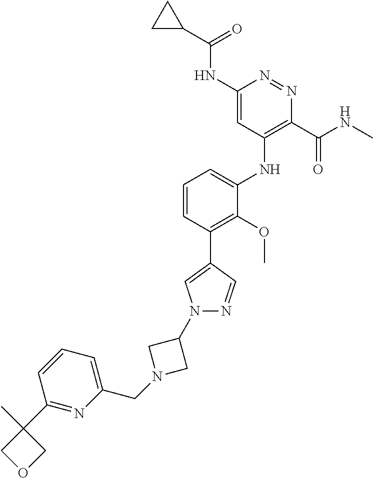 [SMILES: CNC(=O)c1nnc(NC(=O)C2CC2)cc1Nc1cccc(-c2cnn(C3CN(Cc4cccc(C5(C)COC5)n4)C3)c2)c1OC] | 624.3 [M + H]+ | ¹H NMR (400 MHz, Chloroform-d) 11.14 (s, 1H), 8.69 (s, 1H), 8.28 (s, 1H), 8.18 (d, J = 5.3 Hz, 1H), 8.06 (s, 1H), 7.98 (s, 1H), 7.69 (t, J = 7.7 Hz, 1H), 7.37 (dd, J = 7.9, 1.6 Hz, 1H), 7.32 (dd, J = 7.9, 1.6 Hz, 1H), 7.23 (q, J = 8.7, 7.8 Hz, 2H), 7.11 (d, J = 7.8 Hz, 1H), 5.13 (d, J = 5.6 Hz, 3H), 4.66 (d, J = 5.6 Hz, 2H), 4.03 (s, 4H), 3.67 (s, 5H), 3.08 (d, J = 5.0 Hz, 3H), 1.77 (s, 3H), 1.68 (tt, J = 8.1, 4.5 Hz, 1H), 1.18- 1.09 (m, 2H), 1.02-0.91 (m, 2H). |
| 378 | 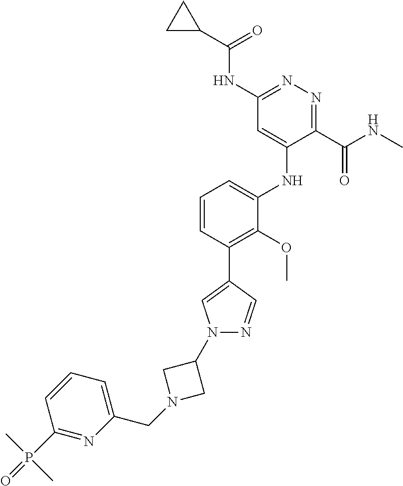 [SMILES: CNC(=O)c1nnc(NC(=O)C2CC2)cc1Nc1cccc(-c2cnn(C3CN(Cc4cccc(P(C)(C)=O)n4)C3)c2)c1OC] | 630.3 [M + H]+ | ¹H NMR (400 MHz, DMSO-d6) 11.31 (s, 1H), 10.98 (s, 1H), 9.17 (d, J = 5.2 Hz, 1H), 8.38 (s, 1H), 8.15 (s, 1H), 8.03 (s, 1H), 7.96 (td, J = 7.6, 3.4 Hz, 1H), 7.84 (t, J = 6.5 Hz, 1H), 7.55 (d, J = 7.9 Hz, 1H), 7.52-7.45 (m, 1H), 7.31 (d, J = 7.7 Hz, 1H), 7.21 (t, J = 7.9 Hz, 1H), 5.14 (t, J = 6.9 Hz, 1H), 3.91 (s, 2H), 3.84 (t, J = 7.4 Hz, 2H), 3.64 (t, J = 7.1 Hz, 2H), 3.60 (s, 3H), 2.87 (d, J = 4.7 Hz, 3H), 2.09 (d, J = 7.9 Hz, 1H), 1.66 (d, J = 13.5 Hz, 6H), 0.87-0.79 (m, 4H). |
| 381 | 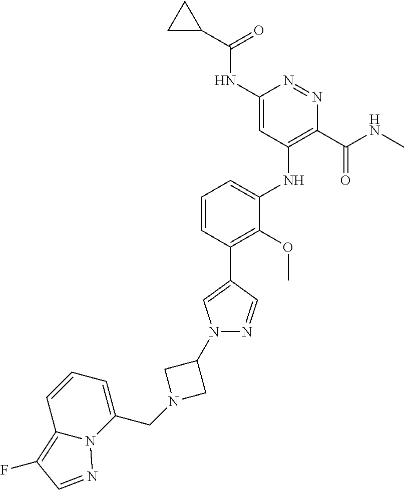 [SMILES: CNC(=O)c1nnc(NC(=O)C2CC2)cc1Nc1cccc(-c2cnn(C3CN(Cc4cccc5c(F)cnn45)C3)c2)c1OC] | 611.2 [M + H]+ | ¹H NMR (400 MHz, DMSO) δ 11.31 (s, 1H), 10.97 (s, 1H), 9.16 (q, J = 4.7 Hz, 1H), 8.42 (s, 1H), 8.16-8.00 (m, 3H), 7.63 (dd, J = 8.9, 1.3 Hz, 1H), 7.47 (dd, J = 7.8, 1.6 Hz, 1H), 7.32-7.15 (m, 3H), 6.94 (dd, J = 6.9, 1.3 Hz, 1H), 5.17 (p, J = 6.9 Hz, 1H), 4.19 (s, 2H), 3.94 (td, J = 7.2, 1.6 Hz, 2H), 3.70 (td, J = 6.6, 1.6 Hz, 2H), 3.59 (s, 3H), 2.85 (d, J = 4.8 Hz, 3H), 2.11-2.02 (m, 1H), 0.87-0.71 (m, 4H). |
| 382 | 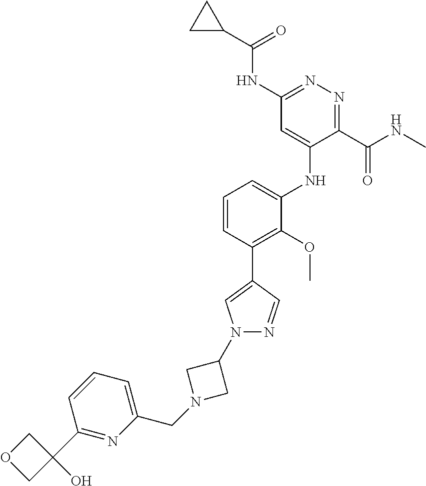 [SMILES: CNC(=O)c1nnc(NC(=O)C2CC2)cc1Nc1cccc(-c2cnn(C3CN(Cc4cccc(C5(O)COC5)n4)C3)c2)c1OC] | 626.2 [M + H]+ | ¹H NMR (400 MHz, DMSO-d6) 11.32 (s, 1H), 10.98 (s, 1H), 9.17 (q, J = 4.8 Hz, 1H), 8.39 (s, 1H), 8.18 (d, J = 16.1 Hz, 2H), 8.04 (s, 1H), 7.80 (t, J = 7.7 Hz, 1H), 7.48 (dd, J = 7.9, 1.7 Hz, 2H), 7.39-7.27 (m, 2H), 7.21 (t, J = 7.9 Hz, 1H), 5.14 (p, J = 6.9 Hz, 1H), 4.93 (d, J = 6.0 Hz, 2H), 4.64 (d, J = 6.0 Hz, 2H), 3.90 (s, 2H), 3.86 (t, J = 7.6 Hz, 2H), 3.65 (td, J = 6.7, 1.5 Hz, 2H), 3.60 (s, 3H), 2.87 (d, J = 4.8 Hz, 3H), 2.13-2.05 (m, 1H), 0.89- 0.78 (m, 4H). |
| 384 | 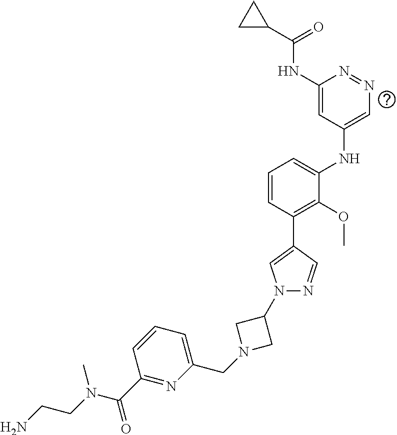 | 654.5 [M + H]+ | ¹H NMR (400 MHz, DMSO-d6) 11.33 (s, 1H), 10.98 (s, 1H), 9.18 (q, J = 4.9 Hz, 1H), 8.37 (d, J = 5.7 Hz, 1H), 8.27-7.88 (m, 4H), 7.66-7.44 (m, 3H), 7.31 (dt, J = 8.1, 2.2 Hz, 1H), 7.21 (td, J = 7.9, 3.8 Hz, 1H), 5.18 (dt, J = 32.0, 7.0 Hz, 1H), 4.02-3.78 (m, 4H), 3.69-3.56 (m, 7H), 3.27 (d, J = 6.0 Hz, 2H), 3.01 (d, J = 23.4 Hz, 3H), 2.87 (d, J = 4.7 Hz, 3H), 2.09 (ddd, J = 9.8, 7.6, 5.0 Hz, 1H), 0.82 (t, J = 6.2 Hz, 4H). |
| 402* | 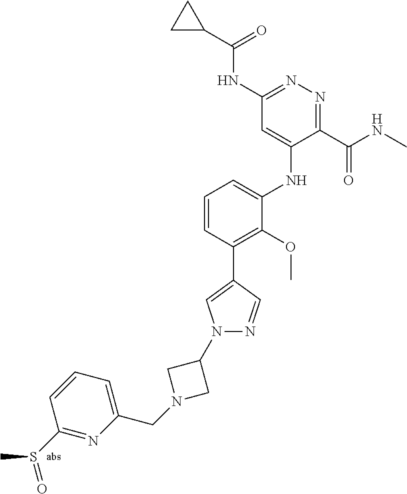 | 616.3 [M + H]+ | ¹H NMR (400 MHz, DMSO-d6) 11.32 (s, 1H), 10.99 (s, 1H), 9.17 (q, J = 4.8 Hz, 1H), 8.39 (s, 1H), 8.16 (s, 1H), 8.11 (td, J = 7.8, 1.4 Hz, 1H), 8.05 (s, 1H), 7.82 (d, J = 7.6 Hz, 1H), 7.57 (d, J = 7.8 Hz, 1H), 7.48 (dd, J = 7.8, 1.6 Hz, 1H), 7.31 (dd, J = 7.9, 1.5 Hz, 1H), 7.21 (t, J = 7.9 Hz, 1H), 5.18 (p, J = 7.0 Hz, 1H), 4.09-3.88 (m, 4H), 3.74 (d, J = 7.2 Hz, 2H), 3.60 (s, 3H), 2.87 (d, J = 4.7 Hz, 3H), 2.81 (s, 3H), 2.13- 2.05 (m, 1H), 0.88-0.79 (m, 4H). |
| 403* | 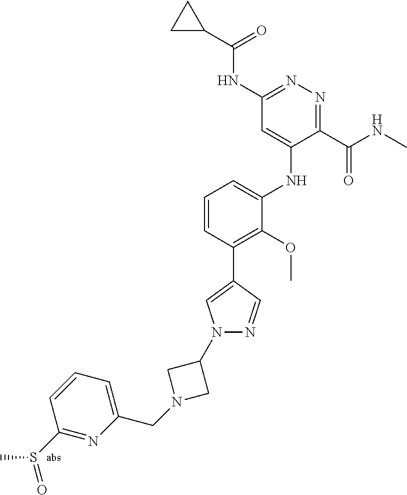 | 616.3 [M + H]+ | ¹H NMR (400 MHz, DMSO-d6) 11.32 (s, 1H), 10.98 (s, 1H), 9.17 (q, J = 4.8 Hz, 1H), 8.39 (s, 1H), 8.18-8.08 (m, 2H), 8.06 (s, 1H), 7.83 (d, J = 7.7 Hz, 1H), 7.58 (d, J = 7.7 Hz, 1H), 7.48 (dd, J = 7.9, 1.6 Hz, 1H), 7.31 (dd, J = 8.0, 1.6 Hz, 1H), 7.21 (t, J = 7.9 Hz, 1H), 5.20 (s, 1H), 3.78 (s, 6H), 3.60 (s, 3H), 2.87 (d, J = 4.8 Hz, 3H), 2.81 (s, 3H), 2.08 (td, J = 7.1, 3.6 Hz, 1H), 0.82 (dq, J = 5.9, 3.8, 3.3 Hz, 4H). |

*Absolute stereochemistry arbitrarily assigned. [symbol]
indicates data missing or illegible when filed

### Example 150

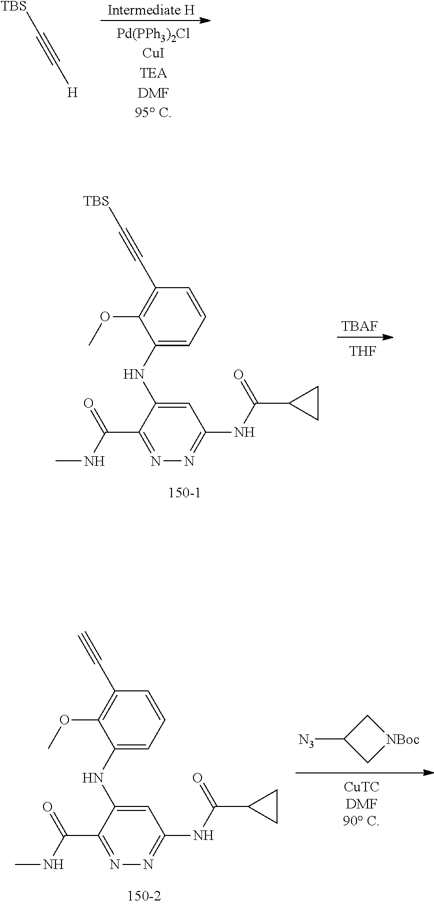

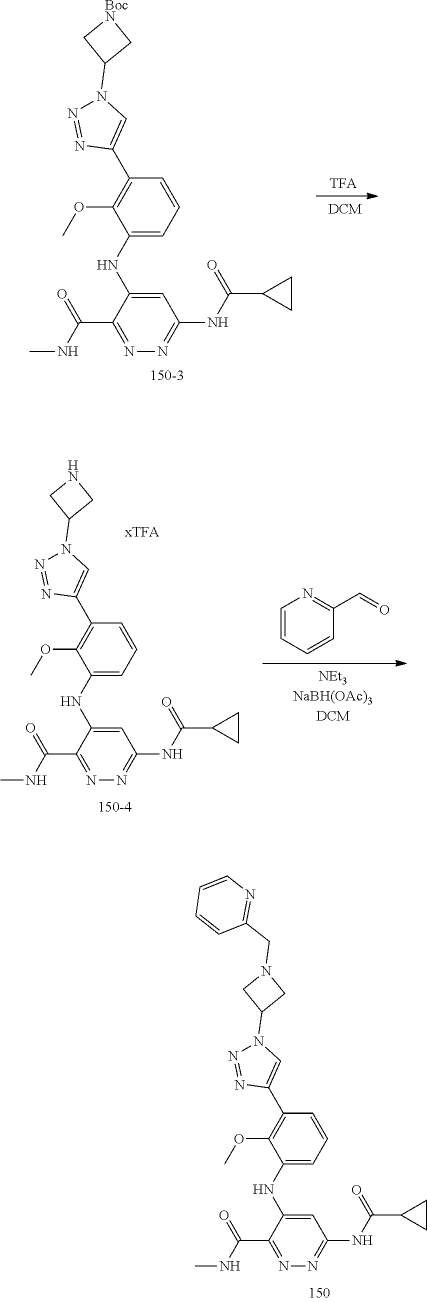 [SMILES: CNC(=O)c1nnc(NC(=O)C2CC2)cc1Nc1cccc(-c2cn(C3CN(C(=O)OC(C)(C)C)C3)nn2)c1OC ; CNC(=O)c1nnc(NC(=O)C2CC2)cc1Nc1cccc(-c2cn(C3CNC3)nn2)c1OC ; O=Cc1ccccn1 ; CNC(=O)c1nnc(NC(=O)C2CC2)cc1Nc1cccc(-c2cn(C3CN(Cc4ccccn4)C3)nn2)c1OC]

Step 1: In a 2-dram vial was charged Intermediate H (2000 mg, 1.0 equiv, 4.759 mmol), tert-butyl(ethynyl)dimethylsilane (1.669 g, 2.5 equiv 11.90 mmol), copper (I) iodide (45.32 mg, 0.05 equiv, 237.9 μmol), and bis-(triphenylphosphino)-palladous chloride (334.0 mg, 0.1 equiv, 475.9 μmol). The vial was evacuated and purged with N₂ after which anhydrous DMF (3 ml) with 0.35 ml of TEA was added and reaction was heated to 95° C. for 2 hrs after which it was determined to be complete by LC/MS analysis. The reaction diluted with EtOAc and plug filtered. The filtrate was purified by flash silica gel chromatography (gradient: ethyl acetate/hexanes). The product fractions were combined and concentrated to provide compound 150-1 as an off-white solid which was used directly in the next step.

LC/MS (ESI+) m/z: 480.4 [M+H]+

¹H NMR (500 MHZ, DMSO) δ 11.31 (s, 1H), 10.92 (s, 1H), 9.14 (q, J=4.7 Hz, 1H), 8.08 (s, 1H), 7.47 (dd, J=8.0, 1.6 Hz, 1H), 7.24 (dd, J=7.8, 1.6 Hz, 1H), 7.16 (t, J=7.9 Hz, 1H), 3.82 (s, 3H), 3.39 (s, 1H), 2.84 (d, J=4.8 Hz, 3H), 2.07 (tt, J=6.9, 5.3 Hz, 1H), 0.98 (s, 9H), 1.00-0.93 (m, 1H), 0.84 (s, 1H), 0.92-0.81 (m, 1H), 0.83-0.78 (m, 4H), 0.18 (s, 6H), 0.15-0.07 (m, 1H).

Step 2: Compound 150-1 was dissolved in 2.5 mL of THF after which tetrabutylammonium fluoride (1M in THF, 2.0 equiv). Stirred for 2 hrs after which complete conversion was observed by LC/MS analysis. The reaction was concentrated and purified by flash silica gel chromatography (gradient: DCM/MeOH). The product fractions were combined and concentrated to provide compound 150-2.

LC/MS (ESI+) m/z: 366.2 [M+H]+

¹H NMR (500 MHZ, DMSO) δ 11.31 (s, 1H), 10.92 (s, 1H), 9.14 (q, J=4.8 Hz, 1H), 8.08 (s, 1H), 7.48 (dd, J=8.0, 1.6 Hz, 1H), 7.27 (dd, J=7.8, 1.5 Hz, 1H), 7.17 (t, J=7.9 Hz, 1H), 4.45 (s, 1H), 3.82 (s, 3H), 2.84 (d, J=4.8 Hz, 3H), 2.07 (tt, J=6.8, 5.5 Hz, 1H), 0.87-0.79 (m, 4H).

Step 3: In a 20 ml vial was charged compound 150-2 (250 mg, 1.0 equiv, 684 μmol), tert-butyl 3-azidoazetidine-1-carboxylate (271 mg, 2.74 mL, 0.5 M, 2.0 equiv, 1.37 mmol), and copper (I) thiophene-2-carboxylate (39.1 mg, 0.3 equiv, 205 μmol). DMF (1.2 mL) was added and reaction heated to 90° C. for 30 min until complete conversion was observed by LC/MS analysis. The reaction was then diluted with EtOAc and washed with water, brined, dried and concentrated. The crude material was purified by flash silica gel column chromatography (gradient: DCM/MeOH) to provide compound 150-3 as a white solid which was used directly in the next step.

LC/MS (ESI+) m/z: 564.4 [M+H]+

¹H NMR (500 MHZ, DMSO) δ 11.30 (s, 1H), 10.95 (s, 1H), 9.16 (q, J=4.8 Hz, 1H), 8.63 (s, 1H), 8.10 (s, 1H), 7.93 (dd, J=7.8, 1.6 Hz, 1H), 7.43 (dd, J=7.9, 1.6 Hz, 1H), 7.30 (t, J=7.9 Hz, 1H), 5.74 (s, 1H), 5.56 (tt, J=8.0, 5.3 Hz, 1H), 4.42 (t, J=8.7 Hz, 2H), 4.27 (s, 2H), 3.65 (s, 3H), 2.86 (d, J=4.8 Hz, 3H), 2.11-2.02 (m, 1H), 1.42 (s, 9H), 0.80 (h, J=3.2 Hz, 4H).

Step 4: Compound 150-3 was dissolved in DCM/TFA (1:1 v/v) and stirred for 1 h after which it concentrated to provide compound 150-4 as a light-colored viscous oil

LC/MS (ESI+) m/z: 464.3 [M+H]+

¹H NMR (500 MHZ, DMSO) δ 11.32 (s, 1H), 10.98 (s, 1H), 9.17 (q, J=4.8 Hz, 1H), 8.97 (s, 1H), 8.69 (s, 1H), 8.10 (s, 1H), 7.96 (dd, J=7.9, 1.5 Hz, 1H), 7.45 (dd, J=8.0, 1.6 Hz, 1H), 7.32 (t, J=7.9 Hz, 1H), 5.78-5.69 (m, 1H), 4.52 (hept, J=5.7 Hz, 4H), 3.67 (s, 3H), 2.86 (d, J=4.8 Hz, 3H), 2.06 (tt, J=7.6, 4.8 Hz, 1H), 0.88-0.75 (m, 4H).

Step 5: In a 20 mL scintillation vial was charged compound 150-4, picolinaldehyde (51.9 mg, 2.0 equiv, 485 μmol) in 1 mL of DCM. TEA was then added (98.1 mg, 135 μL, 4.0 equiv, 970 μmol) and stirred reaction for 20 min. Sodium triacetoxyborohydride (103 mg, 2.0 equiv, 485 μmol) was added portion-wise under continuous stirring for 16 h. The reaction was then diluted with DCM (5 mL) and washed with saturated NaHCO₃. The organic layer was concentrated and purified by flash silica gel chromatography (gradient: DCM/MeOH) to provide compound 150 as a white solid.

LC/MS (ESI+) m/z: 555.0 [M+H]+

¹H NMR (500 MHz, DMSO) δ 11.30 (s, 1H), 10.97 (s, 1H), 9.16 (q, J=4.7 Hz, 1H), 8.63 (s, 1H), 8.50 (ddd, J=4.8, 1.8, 0.9 Hz, 1H), 8.11 (s, 1H), 7.92 (dd, J=7.9, 1.6 Hz, 1H), 7.77 (td, J=7.6, 1.8 Hz, 1H), 7.45-7.38 (m, 2H), 7.33-7.23 (m, 2H), 5.40 (p, J=6.6 Hz, 1H), 3.88 (d, J=7.3 Hz, 4H), 3.72-3.66 (m, 2H), 3.65 (s, 3H), 3.29 (s, 8H), 2.86 (d, J=4.8 Hz, 3H), 2.11-2.02 (m, 1H), 0.85-0.77 (m, 4H).

TABLE 15
The following compounds were prepared using analogous procedures as
those in Example 150:

| | | LCMS m/z | |
|---|---|---|---|
| Compound | Structure | (ES+) | ¹H NMR |
| 151 | 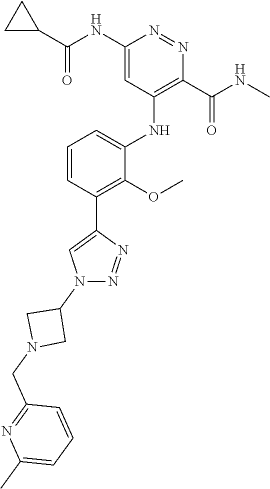 | 569.1 [M + H]+ | ¹H NMR (500 MHz, DMSO) δ 11.30 (s, 1H), 10.97 (s, 1H), 9.16 (q, J = 4.7 Hz, 1H), 8.63 (s, 1H), 8.50 (ddd, J = 4.8, 1.9, 1.0 Hz, 1H), 8.11 (s, 1H), 7.92 (dd, J = 7.9, 1.6 Hz, 1H), 7.76 (td, J = 7.6, 1.8 Hz, 1H), 7.45-7.37 (m, 2H), 7.33-7.22 (m, 2H), 5.39 (p, J = 6.6 Hz, 1H), 3.88 (dd, J = 7.0, 1.5 Hz, 1H), 3.86 (s, 3H), 3.68 (ddd, J = 8.2, 5.3, 2.1 Hz, 2H), 3.65 (s, 3H), 2.86 (d, J = 4.8 Hz, 3H), 2.06 (tt, J = 6.9, 5.4 Hz, 1H), 0.84-0.78 (m, 4H). |
| 183 | 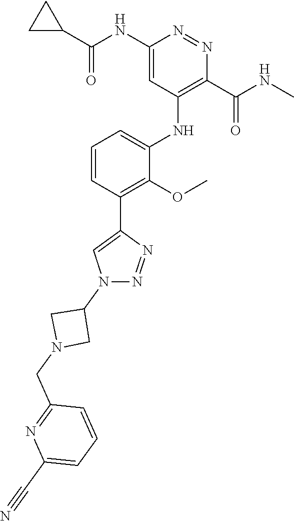 [SMILES: CNC(=O)c1nnc(NC(=O)C2CC2)cc1Nc1cccc(-c2cn(C3CN(Cc4cccc(C#N)n4)C3)nn2)c1OC] | | ¹H NMR (500 MHz, DMSO) δ 11.30 (s, 1H), 10.97 (s, 1H), 9.16 (q, J = 4.7 Hz, 1H), 8.63 (s, 1H), 8.11 (s, 1H), 7.92 (dd, J = 7.9, 1.6 Hz, 1H), 7.64 (t, J = 7.7 Hz, 1H), 7.43 (dd, J = 7.9, 1.7 Hz, 1H), 7.30 (t, J = 7.9 Hz, 1H), 7.19 (d, J = 7.7 Hz, 1H), 7.11 (d, J = 7.6 Hz, 1H), 5.39 (p, J = 6.6 Hz, 1H), 3.90-3.83 (m, 2H), 3.81 (s, 2H), 3.72-3.64 (m, 2H), 3.65 (s, 3H), 2.86 (d, J = 4.8 Hz, 3H), 2.46 (s, 1H), 2.43 (s, 3H), 2.11-2.02 (m, 1H), 0.85- 0.78 (m, 4H). |

### Example 153

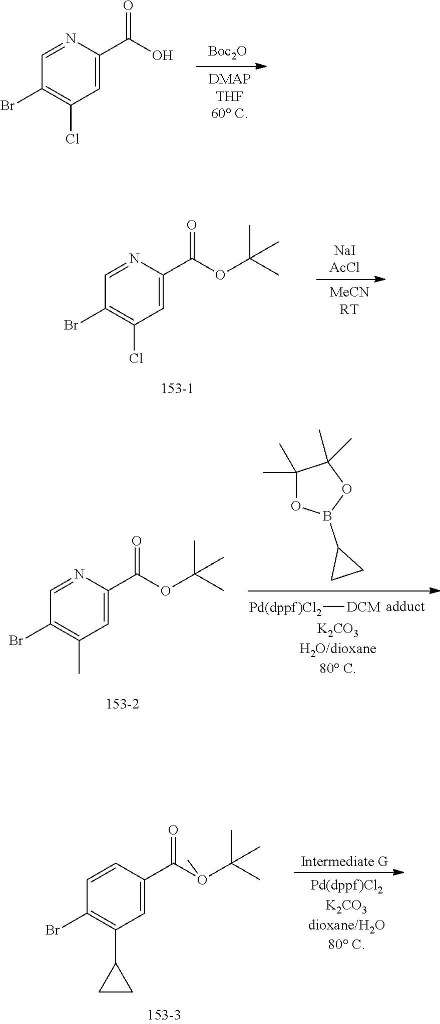

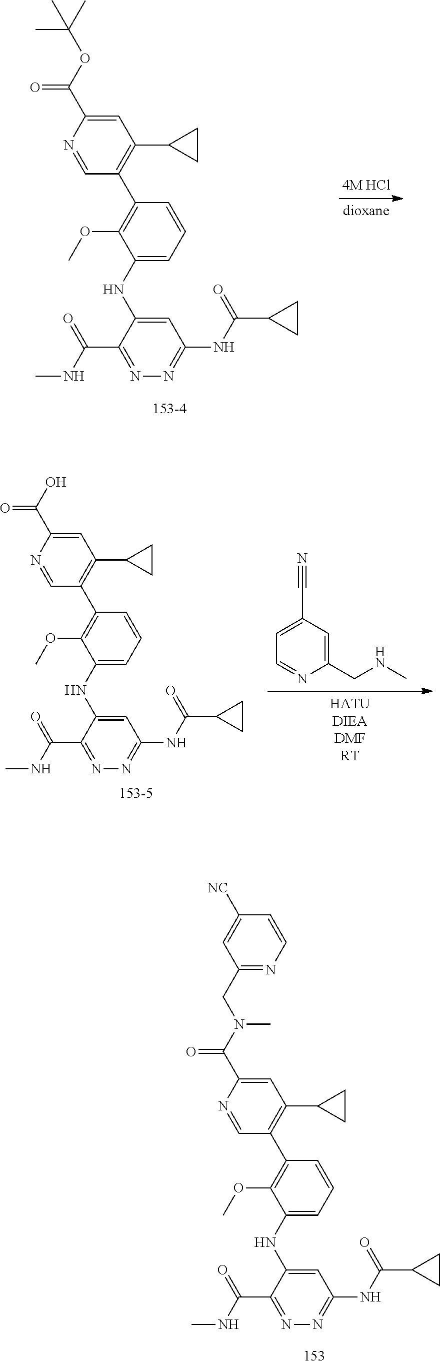 [SMILES: CNC(=O)c1nnc(NC(=O)C2CC2)cc1Nc1cccc(-c2cnc(C(=O)OC(C)(C)C)cc2C2CC2)c1OC ; CNC(=O)c1nnc(NC(=O)C2CC2)cc1Nc1cccc(-c2cnc(C(=O)O)cc2C2CC2)c1OC ; CNCc1cc(C#N)ccn1 ; CNC(=O)c1nnc(NC(=O)C2CC2)cc1Nc1cccc(-c2cnc(C(=O)N(C)Cc3cc(C#N)ccn3)cc2C2CC2)c1OC]

Step 1: A round bottomed flask was charged with 5-bromo-4-chloropicolinic acid (2.0 g, 1.0 equiv, 8 mmol), Boc2O (3.0 g, 3 mL, 1.5 equiv, 0.01 mol), and DMAP (0.1 g, 0.1 equiv, 0.8 mmol). THF (20 mL) was added, and the solution was stirred at 60° C. for 12 hour. The reaction was quenched with water and extracted with ethyl acetate. The organic layer was dried over Na₂SO₄, evaporated and purified using C18 flash chromatography (gradient: water/MeCN) to provide compound 153-1 (1.6 g, 5.5 mmol, 60% yield) as a colorless oil.

LC/MS (ES+) m/z: [M+H]+=291.85

Step 2: A round bottomed flask was charged with compound 153-1 (500 mg, 1.0 equiv, 1.71 mmol), sodium iodide (384 mg, 105 μL, 1.5 equiv, 2.56 mmol). Acetonitrile (5 mL) was added, and the solution was stirred at 25° C. for 30 min. Then AcCl (201 mg, 182 μL, 1.5 equiv, 2.56 mmol) was added, and the solution was stirred at 25° C. for 2 hour. The reaction was quenched with water and extracted with ethyl acetate. The organic layer was washed with Na₂S₂O₃, dried over Na₂SO₄ and evaporated and purified using C18 flash chromatography (gradient: MeCN/water) to provide compound 153-2 (600 mg, 1.56 mmol, 91 yield) as a yellow oil.

LC/MS (ES+) m/z: [M+H]+=383.8

Step 3: A round bottomed flask was charged with compound 153-2 (500 mg, 1.0 equiv, 1.30 mmol), cyclopropylboronic acid (168 mg, 1.5 equiv, 1.95 mmol), K₂CO₃ (540 mg, 3.0 equiv, 3.91 mmol), and PdCl₂(dppf)-CH₂Cl₂ adduct (213 mg, 0.2 equiv, 260 μmol). 1,4-Dioxane (4 mL) and water (0.8 mL) were added, and the solution was stirred at 80° C. for 2 hour under N₂. The reaction was quenched with water and extracted with ethyl acetate. The organic layer was washed with brine, dried over Na₂SO₄ and evaporated, and purified using C18 flash chromatography (gradient: MeCN/water) to provide compound 153-3 (120 mg, 402 μmol, 31% yield) as a yellow oil.

LC/MS (ES+) m/z: [M+H]+=298.10

Step 4: A round bottomed flask was charged with tert-butyl 5-bromo-4-cyclopropylpicolinate (110 mg, 1.0 equiv, 369 μmol), Intermediate G (172 mg, 1.0 equiv, 369 μmol), PdCl₂(dppf)-CH₂Cl₂ adduct (60.3 mg, 0.2 equiv, 73.8 μmol), and K₂CO₃ (153 mg, 3 equiv, 1.11 mmol). 1,4-Dioxane (3 mL) and water (0.6 mL) were added, and the solution was stirred at 80° C. for 2 hours under N₂. The reaction was quenched with water and extracted with ethyl acetate. The organic layer was washed with brine, dried over Na₂SO₄ and evaporated. The crude product was then purified by Prep-TLC (ethyl acetate/petroleum ether=1:1). This resulted in compound 153-4 (140 mg, 251 μmol, 68% yield) as an off-white solid.

LC/MS (ES+) m/z [M+H]+=559.40

Step 5: A round bottomed flask was charged with compound 153-4 (140 mg, 1.0 equiv, 251 μmol) in DCM (3 mL), and 4M HCl in dioxane (3 mL) was added, after which the solution was stirred at 25° C. for 12 hour. The mixture was evaporated to give compound 153-5 (120 mg, 0.19 mmol, 76% yield) as an off-white solid used in next step directly.

LC/MS (ES+) m/z [M+H]+=503.05

Step 6: A round bottomed flask was charged with compound 153-5 (35.1 mg, 1.2 equiv, 239 μmol), HATU (113 mg, 1.5 equiv, 298 μmol), and DIEA (257 mg, 10 Eq, 1.99 mmol). DMF (3 mL) was added, and the solution was stirred at 25° C. for 2 hour. The resulted solution was purified using C18 flash chromatography (gradient: MeCN/Water) Lyophilization yielded compound 153 (73.5 mg, 116 μmol, 59% yield) as an off-white amorphous solid.

LC/MS (ES+) m/z [M+H]+=632.25.

¹H NMR (400 MHz, DMSO-d6) 11.33 (d, J=6.0 Hz, 1H), 10.90 (d, J=14.3 Hz, 1H), 9.16 (t, J=4.8 Hz, 1H), 8.81 (ddd, J=22.3, 4.8, 1.1 Hz, 1H), 8.32 (d, J=54.5 Hz, 1H), 8.05 (d, J=15.5 Hz, 1H), 7.79 (dd, J=14.6, 5.0 Hz, 2H), 7.53 (ddd, J=12.5, 8.0, 1.5 Hz, 1H), 7.32 (dt, J=15.0, 7.8 Hz, 1H), 7.23-6.98 (m, 2H), 4.83 (d, J=17.1 Hz, 2H), 3.37 (s, 1H), 3.30 (s, 2H), 3.07 (d, J=23.5 Hz, 3H), 2.85 (dd, J=4.8, 2.6 Hz, 3H), 2.08 (h, J=4.3 Hz, 1H), 1.86-1.56 (m, 1H), 1.02-0.90 (m, 2H), 0.82 (d, J=5.4 Hz, 6H).

### Example 155

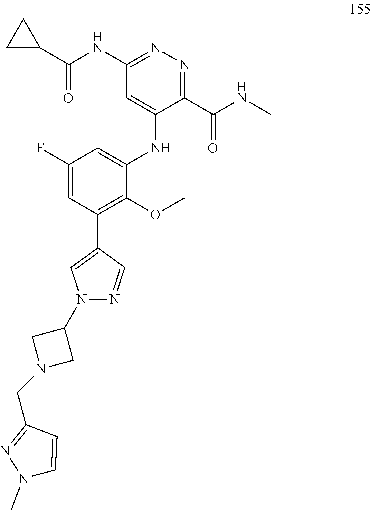 [SMILES: CNC(=O)c1nnc(NC(=O)C2CC2)cc1Nc1cc(F)cc(-c2cnn(C3CN(Cc4ccn(C)n4)C3)c2)c1OC]

Compound 155 was prepared in analogous fashion to related compounds in Example 147 except that Intermediate J was used as starting material.

LC/MS (ESI+) m/z: 575.2 [M+H]+

¹H NMR (400 MHZ, DMSO-d6) 11.39 (s, 1H), 11.16 (s, 1H), 9.22 (d, J=4.9 Hz, 1H), 8.42 (s, 1H), 8.24 (s, 1H), 8.08 (s, 1H), 7.59 (d, J=2.1 Hz, 1H), 7.35 (dd, J=10.0, 3.0 Hz, 1H), 7.18 (dd, J=9.7, 3.0 Hz, 1H), 6.14 (d, J=2.1 Hz, 1H), 5.05 (p, J=7.0 Hz, 1H), 3.79 (s, 3H), 3.72 (t, J=7.4 Hz, 2H), 3.63 (s, 2H), 3.59 (s, 3H), 3.50 (t, J=7.2 Hz, 2H), 2.87 (d, J=4.8 Hz, 3H), 2.10 (p, J=6.3 Hz, 1H), 0.85 (d, J=6.1 Hz, 4H).

### Example 156

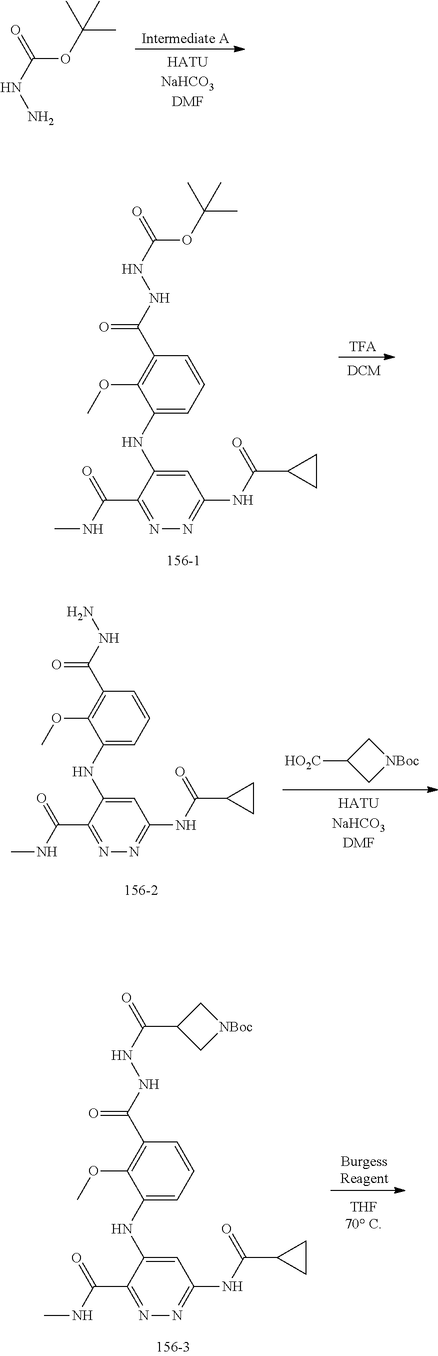

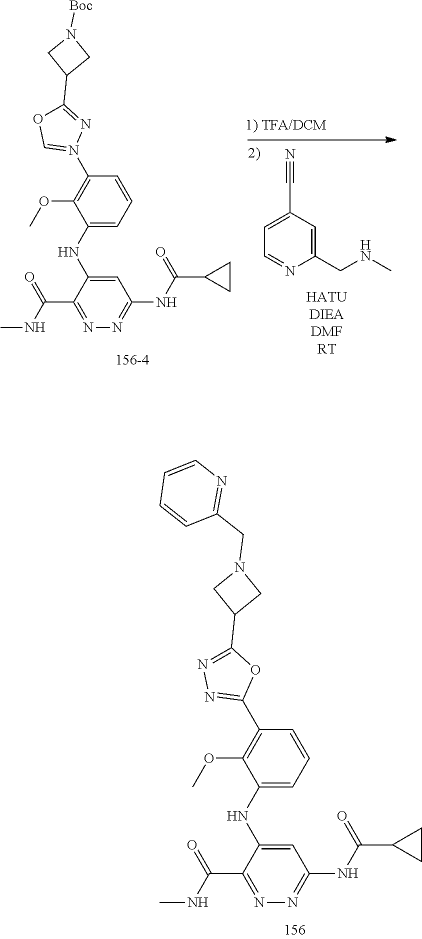

Step 1: In a 40 mL vial was charged Intermediate A (500 mg, 1.0 equiv, 1.30 mmol), tert-butyl hydrazinecarboxylate (206 mg, 1.2 equiv, 1.56 mmol), HATU (888 mg, 1.8 equiv, 2.34 mmol) and sodium bicarbonate (436 mg, 4.0 equiv, 5.19 mmol) in 3 mL of DMF. The reaction stirred at rt for 16 hrs after which it was diluted with EtOAc and water. The organics were washed with water, brine, dried and concentrated. The crude material dry loaded on silica using flash silica gel chromatography (gradient: DCM/MeOH). Product containing fractions were combined and concentrated to a white solid to provide compound 156-1 which was used directly in the next step.

Step 2: Compound 156-1 was dissolved in DCM (2 mL) and TFA (2 mL) was added after which the reaction was concentrated to provide crude compound 156-2 which was used directly in the next step.

Step 3: Compound 156-2, 1-boc-azetidine-3-carboxylic acid (392 mg, 1.5 equiv, 1.95 mmol), HATU (2 equvi) and NaHCO₃ (8 equiv) was stirred in 3 ml of DMF for 16 hrs. The reaction was then diluted with EtOAc and water. The organics were separated and concentrated, dry loaded on silica and purified via flash silica gel chromatography (gradient DCM/MeOH). Product fractions were combined and concentrated to provide compound 156-3 as a white solid.

LC/MS (ESI+) m/z: 583.0

Step 4: In a 30 ml vial was charged 156-3 (250 mg, 1.0 equiv, 429 μmol) and Burgess Reagent (153 mg, 1.5 equiv, 644 μmol) in 3 mL of THF. The reaction heated to 70° C. for 18 hrs after which there was no more SM by LC/MS analysis. The reaction diluted with MTBE and washed with water. The organics were dried and concentrated and purified by flash silica gel chromatography (gradient: DCM/MeOH). The product fractions were combined and concentrated to provide 156-4 as a light-colored oil that was used directly in the next step.

Step 5. Compound 156-4 was dissolved and stirred with 1:1 DCM/TFA (10 mL) after which it was concentrated. The crude material was mixed with the corresponding aldehyde and stirred in DCM, after which 4 eq of TEA (4.0 equiv) and then 2 eq of STAB (2.0 equiv) was added and stirred for 3 hrs. The reaction was diluted with DCM and saturated NaHCO₃. The organics were dried and concentrated and and purified by reverse phase HPLC (gradient: MeCN/water) to provide compound 156.

LC/MS (ES+) m/z: 556:0 [M+H]+

¹H NMR (500 MHz, DMSO) δ 11.34 (s, 1H), 11.07 (s, 1H), 9.18 (q, J=4.8 Hz, 1H), 8.48 (ddd, J=4.8, 1.8, 0.9 Hz, 1H), 8.15 (s, 1H), 7.79-7.68 (m, 3H), 7.44-7.34 (m, 2H), 7.25 (ddd, J=7.5, 4.9, 1.2 Hz, 1H), 4.03 (tt, J=7.9, 6.7 Hz, 1H), 3.80-3.71 (m, 7H), 3.56-3.51 (m, 2H), 3.51-3.37 (m, 1H), 3.27-3.14 (m, 1H), 2.86 (d, J=4.8 Hz, 3H), 2.07 (tt, J=7.0, 5.3 Hz, 1H), 0.82 (dt, J=6.1, 2.4 Hz, 4H)

### Example 157

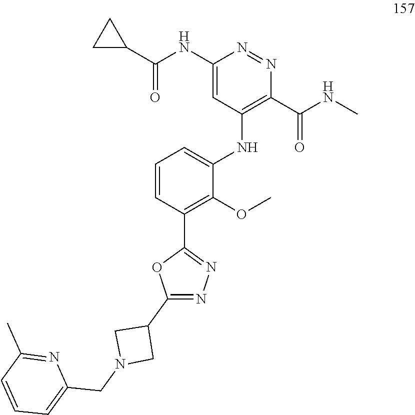 [SMILES: CNC(=O)c1nnc(NC(=O)C2CC2)cc1Nc1cccc(-c2nnc(C3CN(Cc4cccc(C)n4)C3)o2)c1OC]

Compound 157 was prepared using analogous procedures for compound 156 except that 6-methylpicolinaldehyde was used in place of picolinaldehyde.

LC/MS (ES+) m/z: 570.1 [M+H]+

¹H NMR (500 MHZ, DMSO) δ 11.35 (s, 1H), 11.07 (s, 1H), 9.18 (q, J=4.8 Hz, 1H), 8.15 (s, 1H), 7.72 (ddd, J=11.8, 8.0, 1.6 Hz, 2H), 7.63 (t, J=7.7 Hz, 1H), 7.40 (t, J=8.0 Hz, 1H), 7.16 (d, J=7.7 Hz, 1H), 7.10 (d, J=7.6 Hz, 1H), 4.03 (tt, J=7.9, 6.6 Hz, 1H), 3.78 (s, 3H), 3.73 (t, J=7.7 Hz, 2H), 3.71 (s, 2H), 3.52 (t, J=6.9 Hz, 2H), 2.86 (d, J=4.8 Hz, 3H), 2.42 (s, 3H), 2.07 (tt, J=7.0, 5.4 Hz, 1H), 0.86-0.78 (m, 4H).

### Example 159

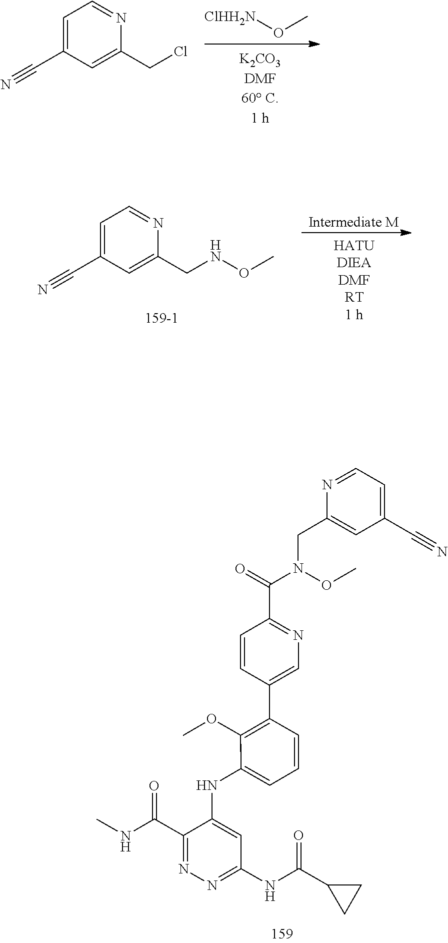

Step 1: A resealable reaction vial was charged with 2-(chloromethyl) isonicotinonitrile (520 mg, 1.0 equiv, 3.41 mmol), O-methylhydroxylamine hydrochloride (569 mg, 2.0 equiv, 6.82 mmol), K₂CO₃ (1.41 g, 3.0 equiv, 10.2 mmol), and a stir bar before being evacuated and purged with nitrogen three times. DMF (12 mL) was added, and the mixture was stirred at 60° C. for 1 h. The reaction mixture was then diluted with H₂O (30 mL), and the aqueous phase was extracted with DCM (3×50 mL) three times. The combined organic layers were washed with brine, dried over sodium sulfate, filtered, and concentrated in vacuo. The resulting crude material was purified by C-18 flash chromatography (gradient: acetonitrile/water) to provide compound 159-1 (55 mg, 10%) as a yellow oil.

LC/MS (ES+) m/z: 164.10 [M+H]+

Step 2: A round bottomed flask was charged with Intermediate M (50 mg, 1.0 equiv, 0.11 mmol), compound 159-1 (21 mg, 1.2 equiv, 0.13 mmol), DIEA (42 mg, 3.0 equiv, 0.32 mmol), HATU (49 mg, 1.2 equiv, 0.13 mmol), and DMF (1 mL) was added, and the solution was stirred at 25° C. for 1 hour. The resulting crude material was purified by prep-HPLC(C18 column, gradient: 0.1% NH₄CO₃ in water/MeCN). Lyophilization of product factions provided compound 159 (35 mg, 53% yield) as a off-white amorphous solid.

LC/MS (ES+) m/z: 608.25 [M+H]+

¹H NMR (400 MHZ, DMSO-d6) 11.35 (s, 1H), 11.00 (s, 1H), 9.18 (d, J=4.8 Hz, 1H), 8.80 (s, 2H), 8.21-8.12 (m, 2H), 7.88 (s, 3H), 7.57-7.51 (m, 1H), 7.36 (d, J=7.9 Hz, 2H), 5.25 (s, 2H), 3.74 (s, 3H), 3.45 (s, 3H), 2.86 (d, J=4.7 Hz, 3H), 2.09 (d, J=5.5 Hz, 1H), 0.83 (s, 4H).

### Example 165

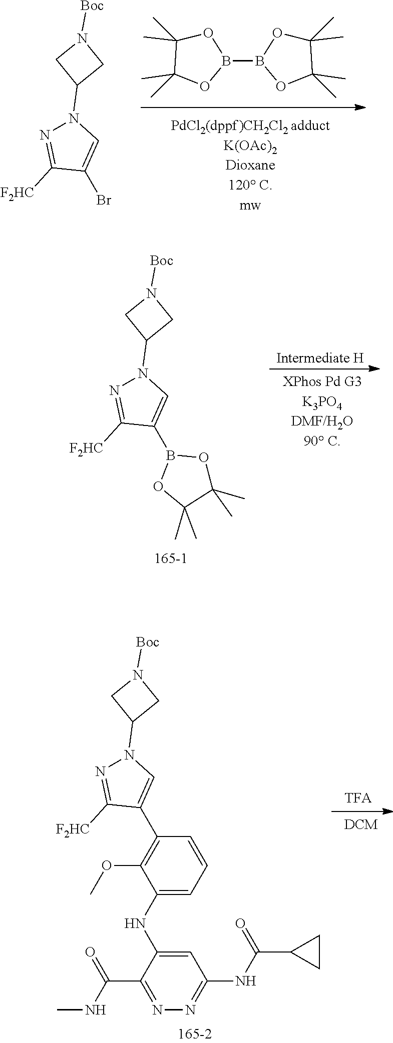

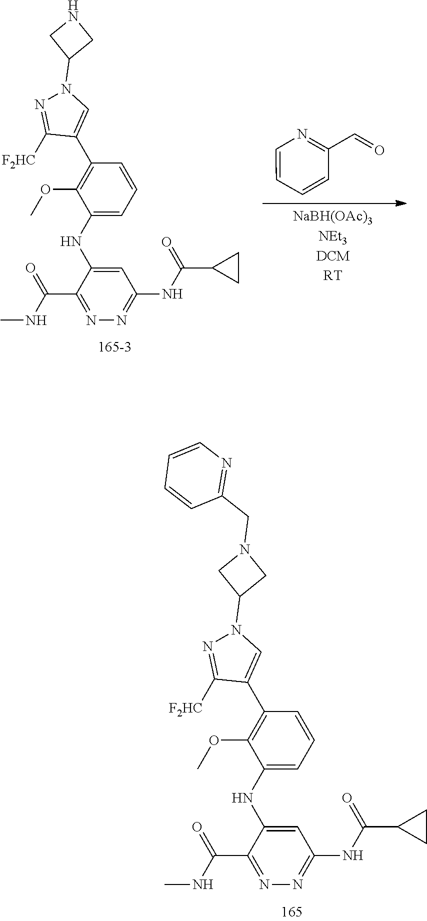 [SMILES: CNC(=O)c1nnc(NC(=O)C2CC2)cc1Nc1cccc(-c2cn(C3CNC3)nc2C(F)F)c1OC ; O=Cc1ccccn1 ; CNC(=O)c1nnc(NC(=O)C2CC2)cc1Nc1cccc(-c2cn(C3CN(Cc4ccccn4)C3)nc2C(F)F)c1OC]

Step 1: A microwave vial was loaded with tert-butyl 3-(4-bromo-3-(difluoromethyl)-1H-pyrazol-1-yl) azetidine-1-carboxylate (100.0 mg, 1.0 equiv, 283.9 μmol), PdCl₂(dppf)-CH₂Cl₂ adduct (69.56 mg, 0.3 equiv, 85.18 μmol), potassium acetate (55.73 mg, 2 Eq, 567.9 μmol) and 4,4,4′,4′,5,5,5′,5′-octamethyl-2,2′-bi(1,3,2-dioxaborolane) (144.2 mg, 2.0 equiv, 567.9 μmol). The vial was purged with nitrogen after which 1,4-dioxane (1 mL) was added. The reaction was heated at 120° C. for 4 h in a microwave reactor until the reaction was determined to be complete by LC/MS analysis. The reaction is cooled down, filtered on a pad of celite, rinsed with EtOAc and concentrated. The crude compound 165-1 is used in the next step without further purification.

Step 2: A screw cap vial was loaded with compound 165-1 (113 mg, 1.0 equiv, 283 μmol), Intermediate H (155 mg, 1.3 equiv, 368 μmol), (s)-(dicyclohexyl(2′,4′,6′-triisopropyl-[1,1′-biphenyl]-2-yl)-15-phosphaneyl) (2′-(methylamino)-[1,1′-biphenyl]-2-yl) palladium (III) methanesulfonate (24.4 mg, 0.1 equiv, 28.3 μmol) and potassium phosphate, tribasic (120 mg, 46.9 μL, 2.0 equiv, 566 μmol). The vial was purged with nitrogen after which N,N-dimethylformamide (2 mL) and water (0.7 mL) was added. The reaction was heated at 90° C. for 16 h after which there was determined to be no more starting material by LC/MS analysis. The reaction was concentrated, resuspended in DMSO filtered and purified by high pressure flash chromatography on C18 (C18 column, Accqprep, gradient: MeCN/0.1% formic acid in water) The product containing fractions were concentrated yielding the desired product compound 165-2 as a beige solid.

LC/MS (ES+) m/z: 613.1 [M+H]+

¹H NMR (400 MHZ, DMSO) δ 11.32 (s, 1H), 10.93 (s, 1H), 9.15 (d, J=5.0 Hz, 1H), 8.23 (d, J=1.2 Hz, 1H), 8.13 (d, J=7.9 Hz, 1H), 7.42 (dd, J=7.8, 1.8 Hz, 1H), 7.35-7.17 (m, 2H), 7.02 (t, J=53.7 Hz, 1H), 5.32 (ddd, J=13.2, 7.9, 5.3 Hz, 1H), 4.34 (t, J=8.6 Hz, 2H), 4.17 (s, 2H), 3.38 (s, 3H), 2.84 (d, J=4.8 Hz, 3H), 2.12-2.05 (m, 1H), 0.82 (d, J=6.2 Hz, 4H).

Step 3: A screw cap vial was loaded with compound 165-2 (35 mg, 1.0 equiv, 57 μmol) followed by addition of DCM (2 mL) and TFA (2 mL). The vial was stirred at RT for 1 h until the reaction showed complete conversion by LC/MS analysis. The reaction was concentrated and the crude compound 165-3 is used without further purification.

LC/MS (ES+) m/z: 513.0 [M+H]+

Step 4: Compound 165-3 was converted to compound 165 using analogous procedures in Example 150.

MS (ES+): m/z=604.2 [M+H]+

### Example 170

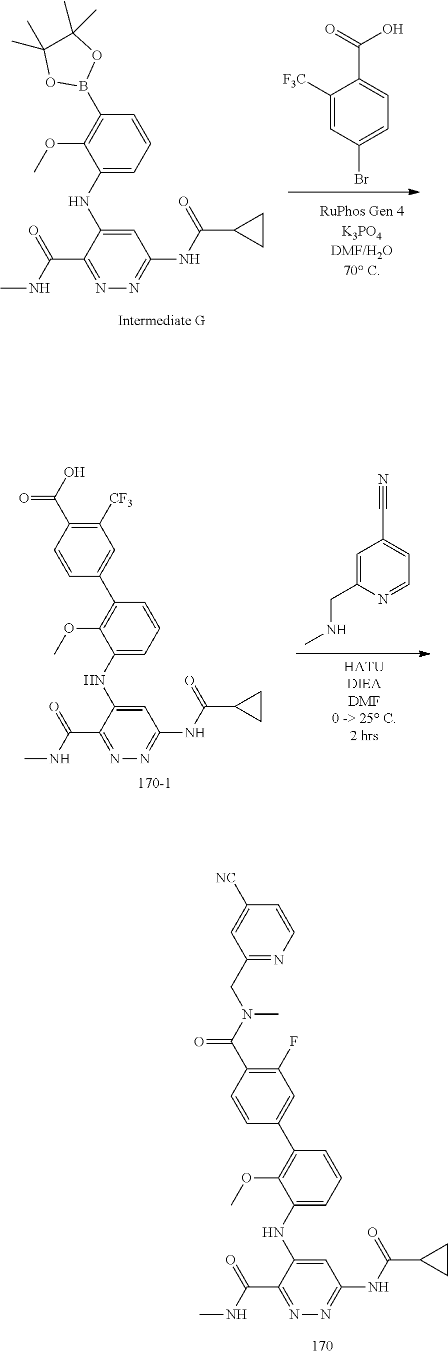 [SMILES: CNC(=O)c1nnc(NC(=O)C2CC2)cc1Nc1cccc(B2OC(C)(C)C(C)(C)O2)c1OC ; O=C(O)c1ccc(Br)cc1C(F)(F)F ; CNC(=O)c1nnc(NC(=O)C2CC2)cc1Nc1cccc(-c2ccc(C(=O)O)c(C(F)(F)F)c2)c1OC ; CNCc1cc(C#N)ccn1 ; CNC(=O)c1nnc(NC(=O)C2CC2)cc1Nc1cccc(-c2ccc(C(=O)N(C)Cc3cc(C#N)ccn3)c(F)c2)c1OC]

Step 1: A screw cap vial was loaded with 4-bromo-2-(trifluoromethyl)benzoic acid (200.0 mg, 1.0 equiv, 743.4 μmol), potassium phosphate (315.6 mg, 2.0 equiv, 1.487 mmol), Intermediate G (416.9 mg, 1.2 equiv, 892.1 μmol) and methanesulfonato (2-dicyclohexylphosphino-2′,6′-di-i-propoxy-1,1′-biphenyl) (2′-methylamino-1,1′-biphenyl-2-yl) palladium (II) (94.83 mg, 0.15 equiv, 111.5 μmol). The vial was degassed with nitrogen and filled with DMF (3.0 mL) and water (1.0 mL) and the reaction was heated at 70° C. for 16 h until the reaction was determined to be complete by LC/MS analysis. The reaction was concentrated, after which the residue was resuspended in 3.0 mL of 9:1 DMSO:formic acid, filtered and purified by high pressure chromatography (C18 column, gradient: MeCN/0.1% formic acid in water). The product containing fractions were concentrated, yielding the desired compound 170-1 as a white solid.

LC/MS (ES+) m/z: 530.3 [M+H]+

Step 2: Compound 170-1 was converted into compound 170 using the analogous procedures in Example 18.

LC/MS (ES+): m/z=659.2 [M+H]+

### Example 171

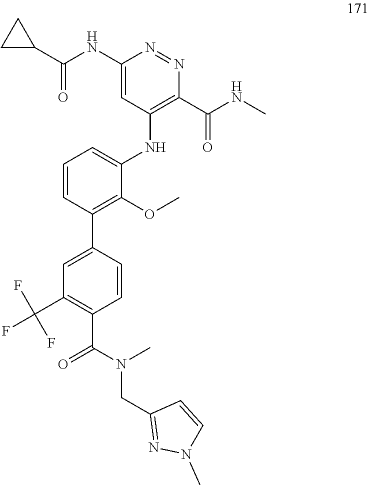 [SMILES: CNC(=O)c1nnc(NC(=O)C2CC2)cc1Nc1cccc(-c2ccc(C(=O)N(C)Cc3ccn(C)n3)c(C(F)(F)F)c2)c1OC]

Compound 171 was prepared using analogous procedures in Example 170 except that N-methyl-1-(1-methyl-1H-pyrazol-3-yl) methanamine was used in place of 2-((methylamino)methyl) isonicotinonitrile.

LC/MS (ES+) m/z=637.2 [M+H]+

### Example 175

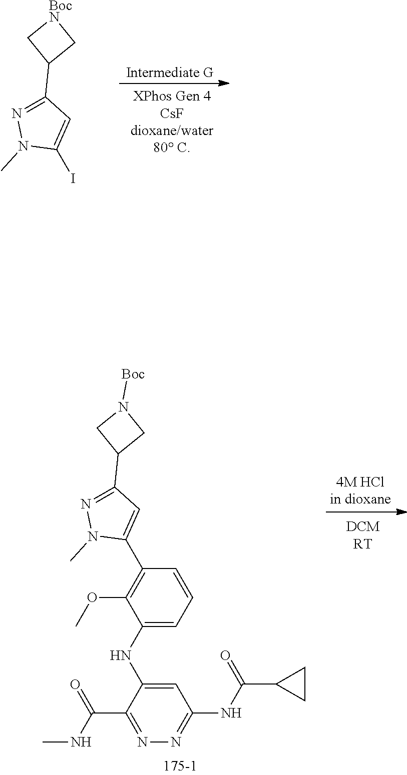 [SMILES: Cn1nc(C2CN(C(=O)OC(C)(C)C)C2)cc1I ; CNC(=O)c1nnc(NC(=O)C2CC2)cc1Nc1cccc(-c2cc(C3CN(C(=O)OC(C)(C)C)C3)nn2C)c1OC]

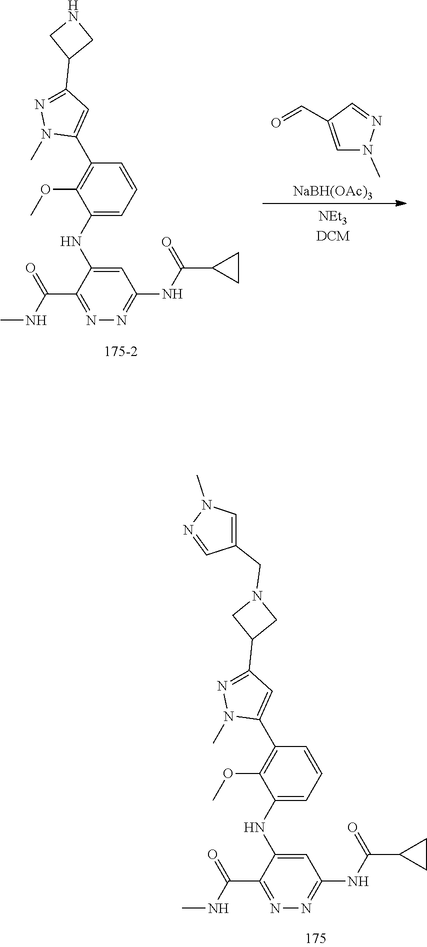

Step 1: A round bottomed flask was charged with tert-butyl 3-(5-iodo-1-methyl-1H-pyrazol-3-yl) azetidine-1-carboxylate (400 mg, 1.0 equiv, 1.10 mmol), Intermediate G (515 mg, 1.0 equiv, 1.10 mmol), cesium fluoride (502 mg, 3.0 equiv, 3.30 mmol), and methanesulfonato (2-dicyclohexylphosphino-2′,4′,6′-tri-i-propyl-1,1′-biphenyl) (2′-methylamino-1,1′-biphenyl-2-yl) palladium (II) (190 mg, 0.2 equiv, 220 μmol). 1,4-Dioxane (5 mL) and water (1 mL) were added, and the solution was stirred at 80° C. for 12 hour under N₂.

The reaction was quenched with water and extracted with ethyl acetate. The organic layer was washed with brine, dried over Na₂SO₄ and evaporated under vacuum. The crude material was purified by prep-TLC (petroleum ether/ethyl acetate: 2:1 v/v) to provide compound 175-1 (350 mg, 607 μmol, 55% yield) as a white solid.

LC/MS (ES+) m/z: 577.30 [M+H]+

Step 2: A round bottomed flask was charged with compound 175-1 (350 mg, 1.0 equiv, 607 μmol) in DCM (3 mL), after which 4M HCl in dioxane (3 mL) was added, and the solution was stirred at 25° C. for 2 hour. The mixture was evaporated to provide compound 175-2 (300 mg, 0.38 mmol, 62% yield) as an colorless oil that was used directly in the next step.

LC/MS (ES+) m/z: 477.25 [M+H]+

Step 3: A mixture of compound 175-2 (60 mg, 1.0 equiv, 0.13 mmol), 1-methyl-1H-pyrazole-4-carbaldehyde (19 mg, 1.4 equiv, 0.18 mmol), TEA (0.10 g, 0.14 mL, 8.0 equiv, 1.0 mmol) were stirred in DCM (3 mL) for 20 min at 25° C. Sodium triacetoxyborohydride (53 mg, 2.0 equiv, 0.25 mmol) was then slowly added, and the mixture was stirred at 25° C. for 30 min. The reaction was quenched with water and extracted with DCM. The organic layer was washed with brine, dried over Na₂SO₄ and evaporated, and purified using HPLC(Xselect CSH C18 OBD Column, gradient: MeCN/0.1% formic acid in water). Lyophilization provided compound 175 (20 mg, 32 μmol, 26% yield) as a white amorphous solid.

LC/MS (ES+) m/z=571.25 [M+H]+

¹H NMR (400 MHZ, Chloroform-d) 11.06 (s, 1H), 9.55 (s, 1H), 8.61 (s, 1H), 8.24 (s, 1H), 8.15 (q, J=5.0 Hz, 1H), 7.65 (s, 1H), 7.58-7.51 (m, 2H), 7.26 (t, J=7.8 Hz, 1H), 7.10 (dd, J=7.7, 1.6 Hz, 1H), 6.20 (s, 1H), 4.25 (t, J=8.8 Hz, 2H), 4.08 (s, 3H), 3.92 (s, 3H), 3.86-3.72 (m, 5H), 3.46 (s, 3H), 3.06 (d, J=5.1 Hz, 3H), 1.80 (tt, J=8.0, 4.5 Hz, 1H), 1.10 (dt, J=6.6, 3.4 Hz, 2H), 0.95 (dq, J=7.6, 4.1 Hz, 2H).

TABLE 16
The following compounds were prepared in analogous fashion to compound
175 in Example 175:

| | | LCMS m/z | |
|---|---|---|---|
| Compound | Structure | (ES+) | ¹H NMR |
| 177 | 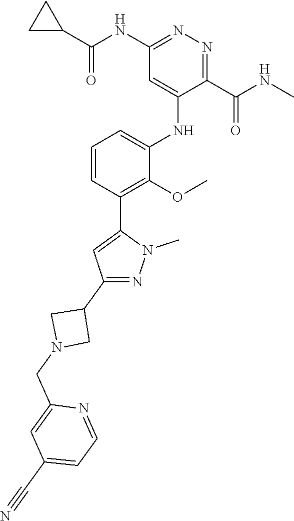 [SMILES: CNC(=O)c1nnc(NC(=O)C2CC2)cc1Nc1cccc(-c2cc(C3CN(Cc4cc(C#N)ccn4)C3)nn2C)c1OC] | 593.3 [M + H]+ | ¹H NMR (400 MHz, Chloroform-d) 11.06 (s, 1H), 8.86 (s, 1H), 8.76 (dd, J = 5.0, 0.9 Hz, 1H), 8.24 (s, 1H), 8.18 (d, J = 5.4 Hz, 1H), 7.73 (s, 1H), 7.54 (dd, J = 8.0, 1.6 Hz, 1H), 7.47-7.41 (m, 1H), 7.25 (d, J = 7.9 Hz, 1H), 7.13 (dd, J = 7.7, 1.6 Hz, 1H), 6.29 (s, 1H), 3.98 (d, J = 33.0 Hz, 5H), 3.76 (s, 3H), 3.56 (s, 2H), 3.48 (s, 3H), 3.07 (d, J = 5.1 Hz, 3H), 1.71 (tt, J = 7.8, 4.5 Hz, 1H), 1.16-1.08 (m, 2H), 0.96 (dt, J = 7.9, 3.5 Hz, 2H). |
| 189 | 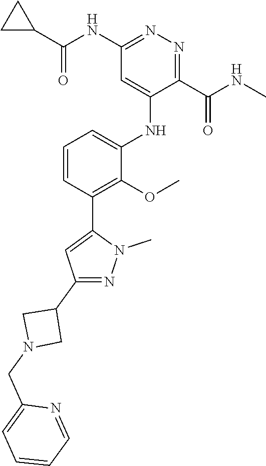 [SMILES: CNC(=O)c1nnc(NC(=O)C2CC2)cc1Nc1cccc(-c2cc(C3CN(Cc4ccccn4)C3)nn2C)c1OC] | 568.4 [M + H]+ | ¹H NMR (400 MHz, Chloroform-d) 11.05 (s, 1H), 8.97 (s, 1H), 8.58 (dt, J = 4.6, 1.5 Hz, 1H), 8.24 (s, 1H), 8.17 (q, J = 5.2 Hz, 1H), 7.69 (td, J = 7.7, 1.8 Hz, 1H), 7.54 (dd, J = 8.0, 1.6 Hz, 1H), 7.44 (d, J = 7.8 Hz, 1H), 7.26 (t, J = 6.9 Hz, 1H), 7.23-7.16 (m, 1H), 7.13 (dd, J = 7.7, 1.6 Hz, 1H), 6.30 (s, 1H), 3.95-3.84 (m, 5H), 3.75 (s, 3H), 3.47 (s, 5H), 3.07 (d, J = 5.1 Hz, 3H), 1.73 (tt, J = 8.0, 4.4 Hz, 1H), 1.12 (dt, J = 6.7, 3.5 Hz, 2H), 0.95 (dq, J = 7.6, 4.1 Hz, 2H). |
| 191 | 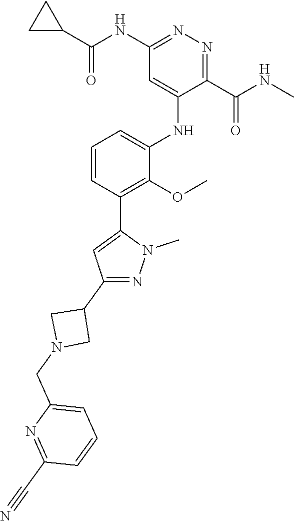 [SMILES: CNC(=O)c1nnc(NC(=O)C2CC2)cc1Nc1cccc(-c2cc(C3CN(Cc4cccc(C#N)n4)C3)nn2C)c1OC] | 593.4 [M + H]+ | ¹H NMR (400 MHz, Chloroform-d) 11.06 (s, 1H), 8.55 (s, 1H), 8.21 (d, J = 6.5 Hz, 2H), 7.90 (s, 2H), 7.67 (s, 1H), 7.54 (dd, J = 8.0, 1.6 Hz, 1H), 7.26 (d, J = 7.9 Hz, 1H), 7.12 (dd, J = 7.7, 1.6 Hz, 1H), 6.27 (s, 1H), 4.07 (d, J = 53.4 Hz, 5H), 3.77 (s, 4H), 3.48 (s, 4H), 3.07 (d, J = 5.2 Hz, 3H), 1.66 (dt, J = 7.9, 3.6 Hz, 1H), 1.12 (p, J = 4.3 Hz, 2H), 0.97 (dq, J = 7.7, 4.1 Hz, 2H). |

### Example 176

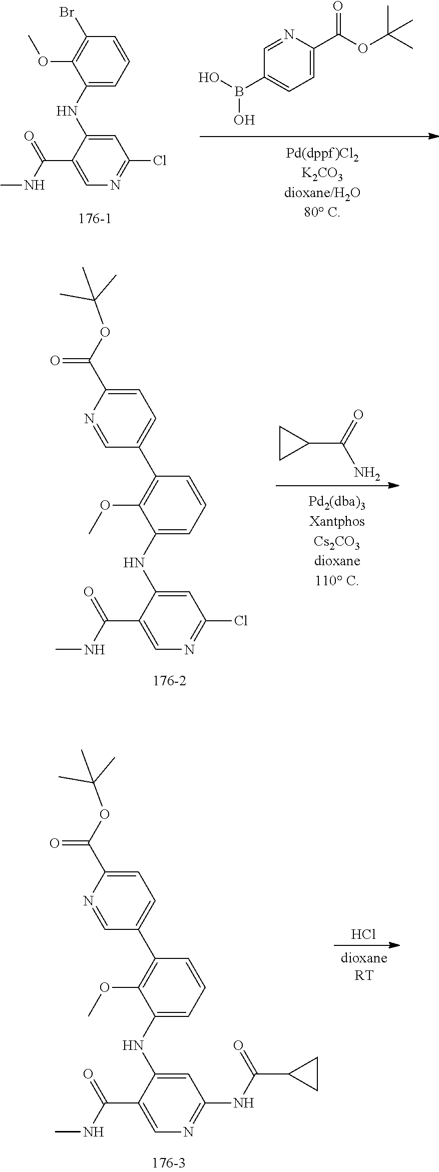 [SMILES: CNC(=O)c1cnc(Cl)cc1Nc1cccc(Br)c1OC ; CC(C)(C)OC(=O)c1ccc(B(O)O)cn1 ; CNC(=O)c1cnc(Cl)cc1Nc1cccc(-c2ccc(C(=O)OC(C)(C)C)nc2)c1OC ; NC(=O)C1CC1 ; CNC(=O)c1cnc(NC(=O)C2CC2)cc1Nc1cccc(-c2ccc(C(=O)OC(C)(C)C)nc2)c1OC ; Cl]

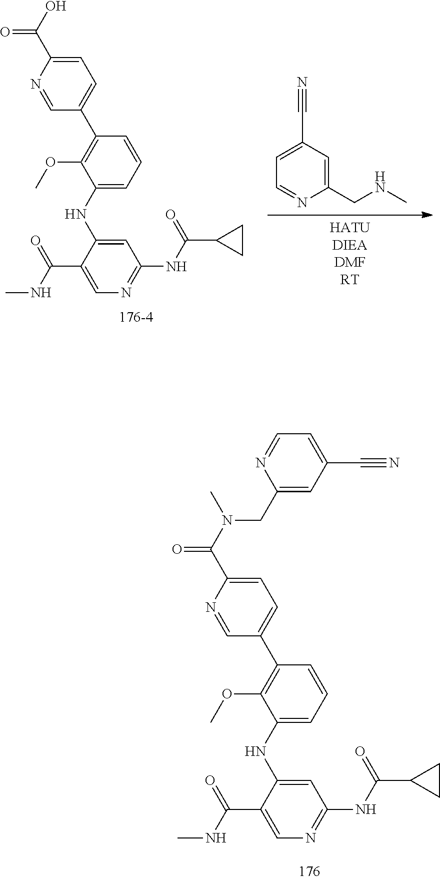 [SMILES: CNC(=O)c1cnc(NC(=O)C2CC2)cc1Nc1cccc(-c2ccc(C(=O)O)nc2)c1OC ; CNCc1cc(C#N)ccn1 ; CNC(=O)c1cnc(NC(=O)C2CC2)cc1Nc1cccc(-c2ccc(C(=O)N(C)Cc3cc(C#N)ccn3)nc2)c1OC]

Step 1: Compound 176-1 was prepared using analogous procedures for the preparation of Intermediate H. A mixture of compound 176-1 (700 mg, 1.0 equiv, 1.89 mmol), (6-(tert-butoxycarbonyl)pyridin-3-yl) boronic acid (379 mg, 0.9 equiv, 1.70 mmol) and potassium carbonate (783 mg, 3.0 equiv, 5.67 mmol) were taken up in 1,4-dioxane (8 mL) and water (2 mL), and nitrogen was bubbled through the slurry for about 10 min. PdCl₂(dppf)-CH₂Cl₂ adduct (154 mg, 0.1 equiv, 189 μmol) was then added with a continued nitrogen flow for an additional 5 min. The reaction heated to 80° C. for 1 hour. After cooling to room temperature, the mixture was purified by flash chromatography on silica gel. Concentration in vacuo resulted compound 176-2 (600 mg, 1.28 mmol, 68% yield) as a yellow solid.

LC/MS (ES+) m/z=469.1 [M+H]+

Step 2: A mixture of compound 176-2 (600 mg, 1.0 equiv, 1.28 mmol), cyclopropanecarboxamide (218 mg, 2.0 equiv, 2.56 mmol) and Cs₂CO₃ (1.25 g, 3.0 equiv, 3.84 mmol) were taken up in 1,4-dioxane (8 mL), and nitrogen bubbled through the slurry for about 10 min. Pd₂(dba)₃ (117 mg, 0.1 equiv, 128 μmol) and xantphos (148 mg, 0.2 equiv, 256 μmol) were then added with a continued nitrogen flow for an additional 5 min. The reaction was heated to 110° C. for 16 hour. After cooling to room temperature, the mixture was purified by flash chromatography on silica gel. Concentration in vacuo resulted in compound 176-3 (400 mg, 773 μmol, 60% yield) as a brown solid.

LC/MS (ES+) m/z: 518.20 [M+H]+

Step 3: Compound 176-3 (350 mg, 1.0 equiv, 676 μmol) was dissolved in DCM (2 mL), and 1,4-dioxane of HCl (5 mL) was then added, the mixture was was stirred at 25° C. for 48 hours. Solvents were evaporated in vacuo to provide compound 176-4 (300 mg, 650 μmol, 96.1%) as a yellow solid.

m/z (ES+) [M+H]+=462.10

Step 4: A solution of compound 176-4 (350 mg, 1.0 equiv, 758 μmol), DIEA (294 mg, 396 μL, 3.0 equiv, 2.28 mmol) and HATU (346 mg, 1.2 equiv, 910 μmol) in DMF (4 mL) were stirred at room temperature for 5 min after which 2-((methylamino)methyl) isonicotinonitrile (112 mg, 1.0 equiv, 758 μmol) was then added, and the reaction was stirred at 25° C. for 1 hour. The resulting residue was purified by flash chromatography on silica gel. Lyophilization yielded compound 176 (276 mg, 467 μmol, 62% yield) as a white amorphous solid.

LC/MS (ES+) m/z=591.2 [M+H]+

¹H NMR (400 MHZ, DMSO-d6) δ 10.86-10.63 (m, 2H), 8.90-8.66 (m, 2H), 8.63 (q, J=4.6 Hz, 1H), 8.53 (d, J=4.3 Hz, 1H), 8.22-8.02 (m, 2H), 7.86-7.67 (m, 3H), 7.56-7.46 (m, 1H), 7.36-7.12 (m, 2H), 4.89 (d, J=12.2 Hz, 2H), 3.38 (d, J=25.0 Hz, 3H), 3.10 (d, J=35.0 Hz, 3H), 2.80 (dd, J=4.5, 3.0 Hz, 3H), 2.05-1.94 (m, 1H), 0.83-0.75 (m, 4H).

### Example 182

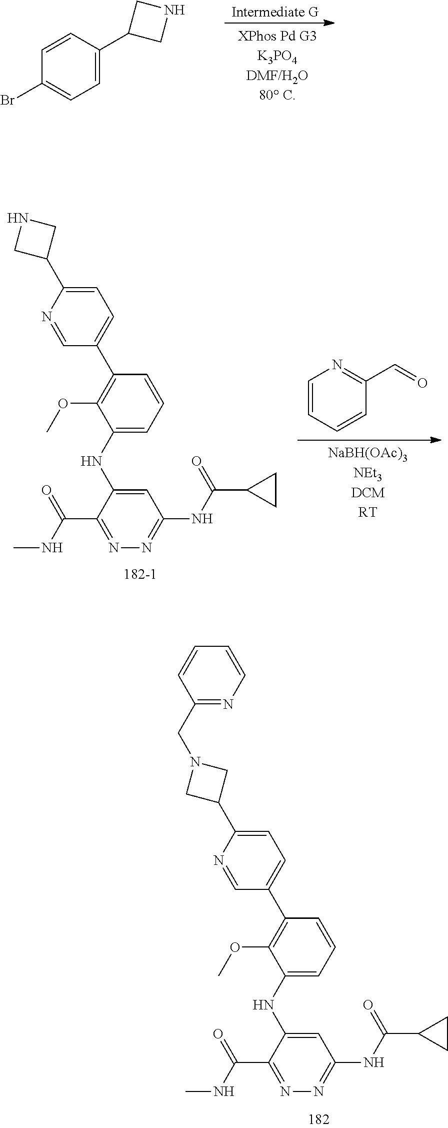 [SMILES: Brc1ccc(C2CNC2)cc1 ; CNC(=O)c1nnc(NC(=O)C2CC2)cc1Nc1cccc(-c2ccc(C3CNC3)nc2)c1OC ; O=Cc1ccccn1 ; CNC(=O)c1nnc(NC(=O)C2CC2)cc1Nc1cccc(-c2ccc(C3CN(Cc4ccccn4)C3)nc2)c1OC]

Step 1: A screw cap vial was loaded with 2-(azetidin-3-yl)-5-bromopyridine HCl (109 mg, 1.2 equiv, 514 μmol), Intermediate G, (200 mg, 1.0 equiv, 428 μmol), (s)-(dicyclohexyl(2′,4′,6′-triisopropyl-[1,1′-biphenyl]-2-yl)-15-phosphaneyl) (2′-(methylamino)-[1,1′-biphenyl]-2-yl) palladium (III) methanesulfonate (36.9 mg, 0.1 equiv, 42.8 μmol) and potassium phosphate, tribasic (318 mg, 124 μL, 3.5 equiv, 1.50 mmol). The vial was purged with nitrogen after which N,N-dimethylformamide (10 mL) and water (3.3 mL) were added. The reaction was heated at 80° C. After 24 h the reaction was concentrated, resuspended in DMSO and formic acid and purified by high pressure flash chromatography (C18, gradient: MeCN/0.1% formic acid in water). The product containing fractions were concentrated to provide compound 182-1 as a white solid.

LC/MS (ES+) m/z: 474.0 [M+H]+

Step 2: A mixture of compound 182-1 (40 mg, 1.0 equiv, 84 μmol), picolinaldehyde (18 mg, 2.0 equiv, 0.17 mmol) and triethylamine (43 mg, 59 μL, 5.0 equiv, 0.42 mmol) were stirred in DCM (2 mL) for 20 minutes at RT before slow addition of sodium triacetoxyborohydride (54 mg, 3.0 equiv, 0.25 mmol) over 20 minutes. The reaction was stirred at RT for 16 h, after which the reaction was determined to be complete by LC/MS analysis. The reaction is concentrated, resuspended in DMSO and purified by high pressure chromatography (C18 gradient MeCN/0.1% formic acid in water). The product containing fractions were concentrated to provide compound 182 as a white solid.

LC/MS (ES+) m/z=565.5 [M+H]+

TABLE 17
The following compounds were prepared using the analogous procedures for
the preparation for compound 182 in Example 182:

| | | LCMS m/z | |
|---|---|---|---|
| Compound | Structure | (ES+) | ¹H NMR |
| 263 | 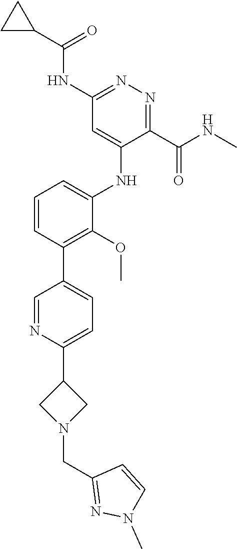 [SMILES: CNC(=O)c1nnc(NC(=O)C2CC2)cc1Nc1cccc(-c2ccc(C3CN(Cc4ccn(C)n4)C3)nc2)c1OC] | 568.3 [M + H]+ | ¹H NMR (400 MHz, DMSO-d6) 11.35 (s, 1H), 10.99 (s, 1H), 9.18 (q, J = 4.8 Hz, 1H), 8.72 (dd, J = 2.4, 0.8 Hz, 1H), 8.20 (s, 1H), 7.94 (dd, J = 8.1, 2.3 Hz, 1H), 7.58 (d, J = 2.1 Hz, 1H), 7.53- 7.43 (m, 2H), 7.35-7.23 (m, 2H), 6.13 (d, J = 2.2 Hz, 1H), 3.78 (s, 4H), 3.65 (t, J = 7.3 Hz, 2H), 3.57 (s, 2H), 3.38 (d, J = 14.0 Hz, 5H), 2.86 (d, J = 4.8 Hz, 3H), 2.09 (td, J = 6.5, 4.4 Hz, 1H), 0.93-0.74 (m, 4H). |
| 264 | 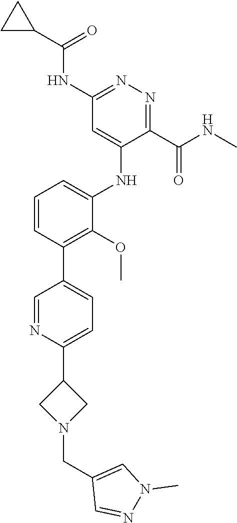 [SMILES: CNC(=O)c1nnc(NC(=O)C2CC2)cc1Nc1cccc(-c2ccc(C3CN(Cc4cnn(C)c4)C3)nc2)c1OC] | 568.3 [M + H]+ | ¹H NMR (400 MHz, DMSO-d6) 11.35 (s, 1H), 10.99 (s, 1H), 9.18 (q, J = 4.7 Hz, 1H), 8.71 (dd, J = 2.3, 0.8 Hz, 1H), 8.20 (s, 1H), 7.94 (dd, J = 8.1, 2.3 Hz, 1H), 7.58 (s, 1H), 7.53-7.43 (m, 2H), 7.37-7.19 (m, 3H), 3.79 (s, 4H), 3.63-3.54 (m, 2H), 3.29 (t, J = 7.3 Hz, 2H), 2.86 (d, J = 4.8 Hz, 3H), 2.17-2.05 (m, 1H), 0.90- 0.70 (m, 4H). |
| 266 | 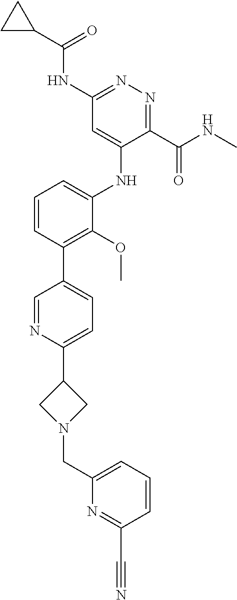 [SMILES: CNC(=O)c1nnc(NC(=O)C2CC2)cc1Nc1cccc(-c2ccc(C3CN(Cc4cccc(C#N)n4)C3)nc2)c1OC] | | ¹H NMR (400 MHz, DMSO-d6) 11.35 (s, 1H), 10.99 (s, 1H), 9.18 (d, J = 4.9 Hz, 1H), 8.74 (d, J = 2.3 Hz, 1H), 8.20 (s, 1H), 8.05 (t, J = 7.8 Hz, 1H), 7.95 (td, J = 8.2, 1.8 Hz, 2H), 7.77 (dd, J = 8.0, 1.1 Hz, 1H), 7.50 (dd, J = 7.7, 1.3 Hz, 2H), 7.37-7.24 (m, 2H), 4.05-3.83 (m, 3H), 3.77 (t, J = 7.2 Hz, 2H), 3.51 (t, J = 6.9 Hz, 2H), 3.39 (s, 3H), 2.86 (d, J = 4.8 Hz, 3H), 2.10 (h, J = 5.7, 5.3 Hz, 1H), 0.92-0.69 (m, 4H). |
| 279 | 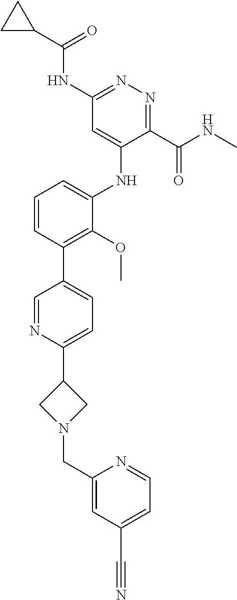 [SMILES: CNC(=O)c1nnc(NC(=O)C2CC2)cc1Nc1cccc(-c2ccc(C3CN(Cc4cc(C#N)ccn4)C3)nc2)c1OC] | 590.2 [M + H]+ | ¹H NMR (400 MHz, DMSO-d6) 11.35 (s, 1H), 10.99 (s, 1H), 9.19 (d, J = 5.0 Hz, 1H), 8.76 (dd, J = 15.0, 3.7 Hz, 2H), 8.20 (s, 1H), 7.96 (dd, J = 8.1, 2.4 Hz, 1H), 7.82 (s, 1H), 7.76 (dd, J = 5.0, 1.6 Hz, 1H), 7.51 (dd, J = 7.9, 1.9 Hz, 2H), 7.38-7.18 (m, 2H), 4.01-3.82 (m, 3H), 3.78 (t, J = 7.3 Hz, 2H), 3.51 (s, 2H), 3.39 (s, 3H), 2.86 (d, J = 4.7 Hz, 3H), 2.10 (p, J = 6.4 Hz, 1H), 0.93- 0.70 (m, 4H). |
| 288 | 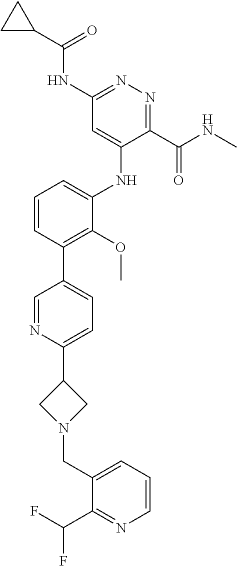 [SMILES: CNC(=O)c1nnc(NC(=O)C2CC2)cc1Nc1cccc(-c2ccc(C3CN(Cc4cccnc4C(F)F)C3)nc2)c1OC] | 615.3 [M + H]+ | ¹H NMR (400 MHz, Chloroform-d) 11.11 (s, 1H), 8.84 (d, J = 19.7 Hz, 2H), 8.57 (d, J = 4.7 Hz, 1H), 8.31 (s, 1H), 8.17 (d, J = 5.3 Hz, 1H), 8.04 (s, 1H), 7.96 (dd, J = 8.1, 2.3 Hz, 1H), 7.51 (dd, J = 7.9, 1.6 Hz, 1H), 7.44 (s, 1H), 7.37 (d, J = 8.1 Hz, 1H), 7.31 (d, J = 7.8 Hz, 1H), 7.19 (dd, J = 7.7, 1.6 Hz, 1H), 6.92 (t, J = 54.6 Hz, 1H), 3.48 (s, 10H), 3.07 (d, J = 5.1 Hz, 3H), 1.72 (tt, J = 8.0, 4.5 Hz, 1H), 1.19 ••C 1.11 (m, 2H), 0.97 (dq, J = 7.5, 4.1 Hz, 2H). |
| 291 | 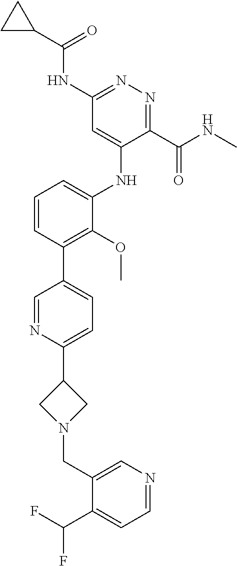 [SMILES: CNC(=O)c1nnc(NC(=O)C2CC2)cc1Nc1cccc(-c2ccc(C3CN(Cc4cnccc4C(F)F)C3)nc2)c1OC] | 615.2 [M + H] | ¹H NMR (400 MHz, Chloroform-d) 11.11 (s, 1H), 8.98 (s, 1H), 8.81 (d, J = 2.2 Hz, 1H), 8.71 (d, J = 4.7 Hz, 2H), 8.31 (s, 1H), 8.17 (d, J = 5.3 Hz, 1H), 7.95 (dd, J = 8.1, 2.3 Hz, 1H), 7.58-7.46 (m, 2H), 7.36- 7.29 (m, 2H), 7.28 (s, 1H), 7.19 (dd, J = 7.8, 1.6 Hz, 1H), 3.91 (s, 3H), 3.80 (s, 2H), 3.54 (s, 2H), 3.48 (s, 3H), 3.07 (d, J = 5.1 Hz, 3H), 1.74 (tt, J = 8.0, 4.5 Hz, 1H), 1.19-1.11 (m, 2H), 0.96 (dq, J = 7.6, 4.1 Hz, 2H). |
| 293 | 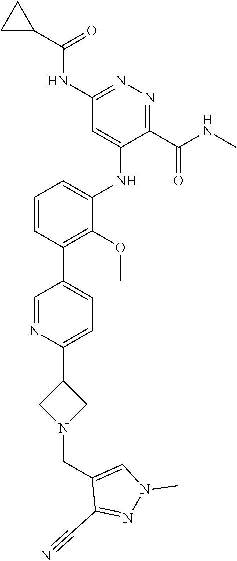 [SMILES: CNC(=O)c1nnc(NC(=O)C2CC2)cc1Nc1cccc(-c2ccc(C3CN(Cc4cn(C)nc4C#N)C3)nc2)c1OC] | 593.3 [M + H]+ | ¹H NMR (400 MHz, DMSO-d6) 11.34 (s, 1H), 10.98 (s, 1H), 9.17 (d, J = 6.1 Hz, 1H), 8.72 (s, 1H), 8.19 (s, 1H), 7.94 (d, J = 7.8 Hz, 2H), 7.49 (t, J = 8.6 Hz, 2H), 7.31 (t, J = 7.7 Hz, 1H), 7.26 (d, J = 7.9 Hz, 1H), 3.91 (s, 3H), 3.82 (t, J = 7.8 Hz, 1H), 3.64 (t, J = 7.0 Hz, 6H), 3.57 (s, 3H), 2.86 (d, J = 4.7 Hz, 3H). |
| 330 | 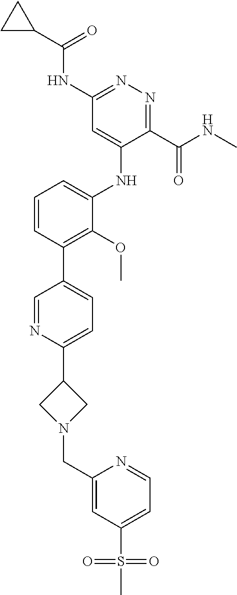 [SMILES: CNC(=O)c1nnc(NC(=O)C2CC2)cc1Nc1cccc(-c2ccc(C3CN(Cc4cc(S(C)(=O)=O)ccn4)C3)nc2)c1OC] | 543.3 [M + H]+ | ¹H NMR (Chloroform-d, 400 MHz) 11.11 (1H, s), 8.94 (1H, s), 8.91-8.81 (2H, m), 8.30 (1H, s), 8.18 (1H, d, J = 5.3 Hz), 8.04 (1H, s), 7.96 (1H, dd, J = 8.0, 2.3 Hz), 7.76 (1H, dd, J = 5.1, 1.7 Hz), 7.52 (1H, dd, J = 8.0, 1.6 Hz), 7.31 (2H, d, J = 7.8 Hz), 7.19 (1H, dd, J = 7.8, 1.5 Hz), 4.34- 4.04 (5H, m), 3.90 (2H, s), 3.48 (3H, s), 3.17 (3H, s), 3.07 (3H, d, J = 5.0 Hz), 1.74 (1H, tt, J = 8.0, 4.5 Hz), 1.14 (2H, dt, J = 6.8, 3.4 Hz), 0.96 (2H, dq, J = 7.5, 4.1 Hz). |
| 385 | 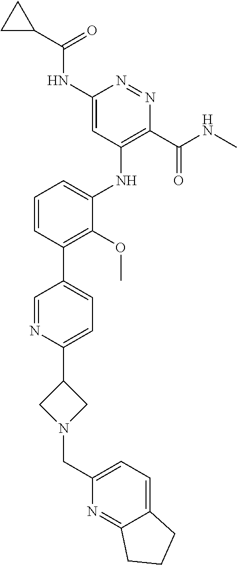 [SMILES: CNC(=O)c1nnc(NC(=O)C2CC2)cc1Nc1cccc(-c2ccc(C3CN(Cc4ccc5c(n4)CCC5)C3)nc2)c1OC] | 605.3 [M + H]+ | |
| 386 | 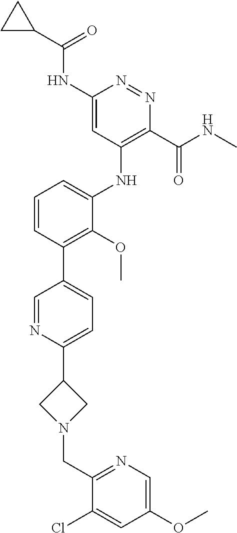 [SMILES: CNC(=O)c1nnc(NC(=O)C2CC2)cc1Nc1cccc(-c2ccc(C3CN(Cc4ncc(OC)cc4Cl)C3)nc2)c1OC] | 629.2 [M + H]+ | |
| 387 | 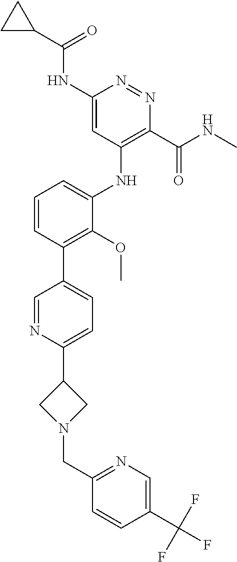 [SMILES: CNC(=O)c1nnc(NC(=O)C2CC2)cc1Nc1cccc(-c2ccc(C3CN(Cc4ccc(C(F)(F)F)cn4)C3)nc2)c1OC] | 633.2 [M + H]+ | |
| 388 | 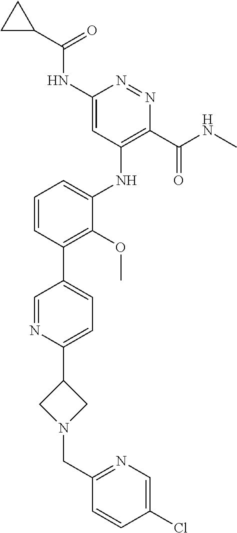 [SMILES: CNC(=O)c1nnc(NC(=O)C2CC2)cc1Nc1cccc(-c2ccc(C3CN(Cc4ccc(Cl)cn4)C3)nc2)c1OC] | 599.2 [M + H]+ | |
| 389 | 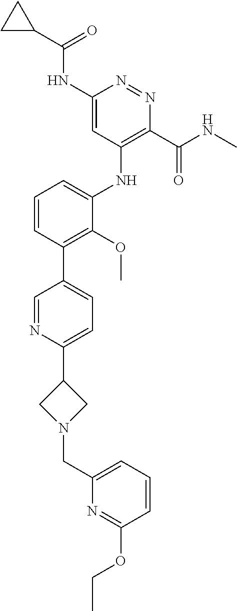 [SMILES: CCOc1cccc(CN2CC(c3ccc(-c4cccc(Nc5cc(NC(=O)C6CC6)nnc5C(=O)NC)c4OC)cn3)C2)n1] | 609.2 [M + H]+ | |
| 390 | 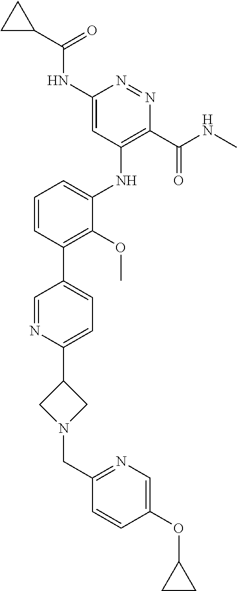 [SMILES: CNC(=O)c1nnc(NC(=O)C2CC2)cc1Nc1cccc(-c2ccc(C3CN(Cc4ccc(OC5CC5)cn4)C3)nc2)c1OC] | 621.3 [M + H]+ | |
| 391 | 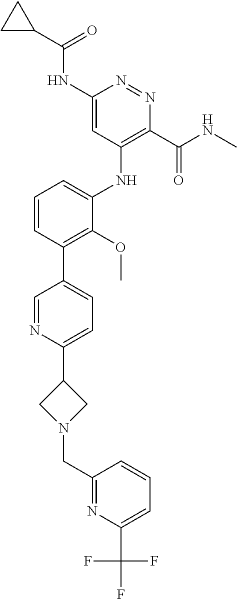 [SMILES: CNC(=O)c1nnc(NC(=O)C2CC2)cc1Nc1cccc(-c2ccc(C3CN(Cc4cccc(C(F)(F)F)n4)C3)nc2)c1OC] | 633.2 [M + H]+ | |

### Example 184

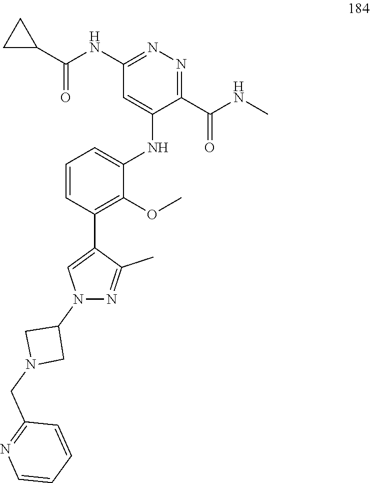 [SMILES: CNC(=O)c1nnc(NC(=O)C2CC2)cc1Nc1cccc(-c2cn(C3CN(Cc4ccccn4)C3)nc2C)c1OC]

Compound 184 was prepared in analogous procedures as compounds in Example 147 except that tert-butyl 3-(3-methyl-4-(4,4,5,5-tetramethyl-1,3,2-dioxaborolan-2-yl)-1H-pyrazol-1-yl) azetidine-1-carboxylate was used to prepare starting material.

LC/MS (ES+) m/z=568.3 [M+H]

### Example 185

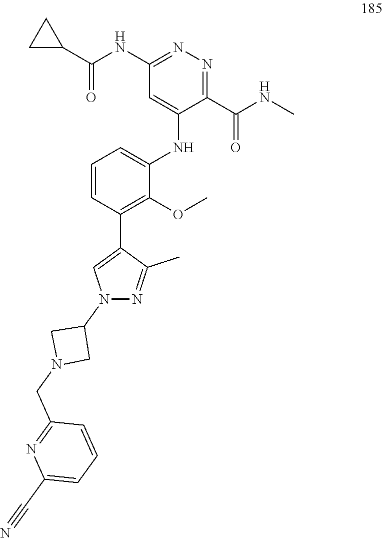 [SMILES: CNC(=O)c1nnc(NC(=O)C2CC2)cc1Nc1cccc(-c2cn(C3CN(Cc4cccc(C#N)n4)C3)nc2C)c1OC]

Compound 185 was prepared in analogous procedures as compounds in Example 147 except that tert-butyl 3-(3-methyl-4-(4,4,5,5-tetramethyl-1,3,2-dioxaborolan-2-yl)-1H-pyrazol-1-yl) azetidine-1-carboxylate was used to prepare starting material.

¹H NMR (500 MHZ, DMSO) δ 11.28 (s, 1H), 10.93 (s, 1H), 9.12 (q, J=4.8 Hz, 1H), 8.15 (s, 1H), 7.94 (s, 1H), 7.68 (s, 1H), 7.37 (dd, J=8.0, 1.5 Hz, 1H), 7.21 (t, J=7.9 Hz, 1H), 7.07 (dd, J=7.7, 1.6 Hz, 1H), 5.74 (s, 1H), 5.27 (tt, J=7.9, 5.4 Hz, 1H), 4.28 (d, J=7.7 Hz, 1H), 4.21 (s, 2H), 3.37 (s, 1H), 3.36 (s, 3H), 2.90-2.81 (m, 6H), 2.72 (d, J=0.6 Hz, 3H), 2.22 (s, 3H), 2.07 (ddd, J=12.5, 7.4, 5.2 Hz, 1H), 1.41 (s, 9H), 0.86-0.78 (m, 4H)

LC/MS (ES+) m/z=593.4 [M+H]

### Example 186

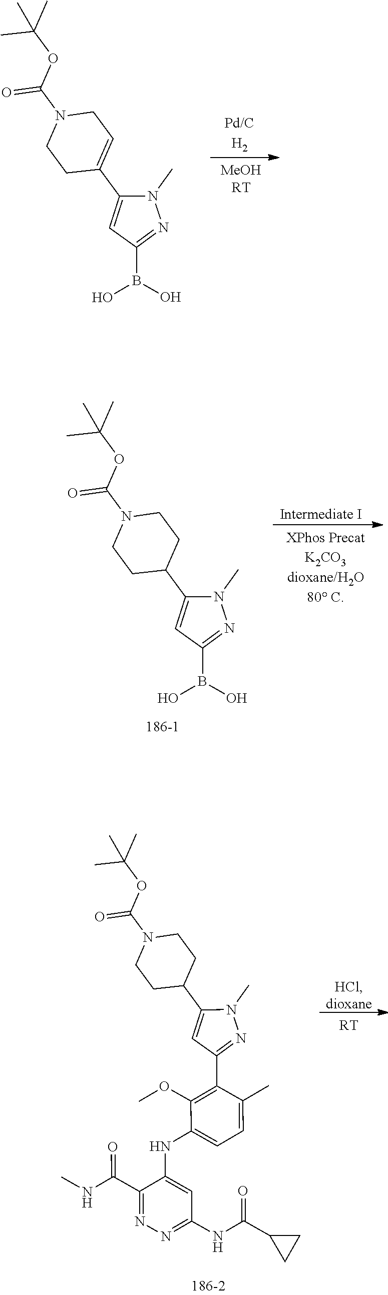 [SMILES: "MeOHRT" ; Cn1nc(B(O)O)cc1C1=CCN(C(=O)OC(C)(C)C)CC1 ; Cn1nc(B(O)O)cc1C1CCN(C(=O)OC(C)(C)C)CC1 ; CNC(=O)c1nnc(NC(=O)C2CC2)cc1Nc1ccc(C)c(-c2cc(C3CCN(C(=O)OC(C)(C)C)CC3)n(C)n2)c1OC ; "RT"]

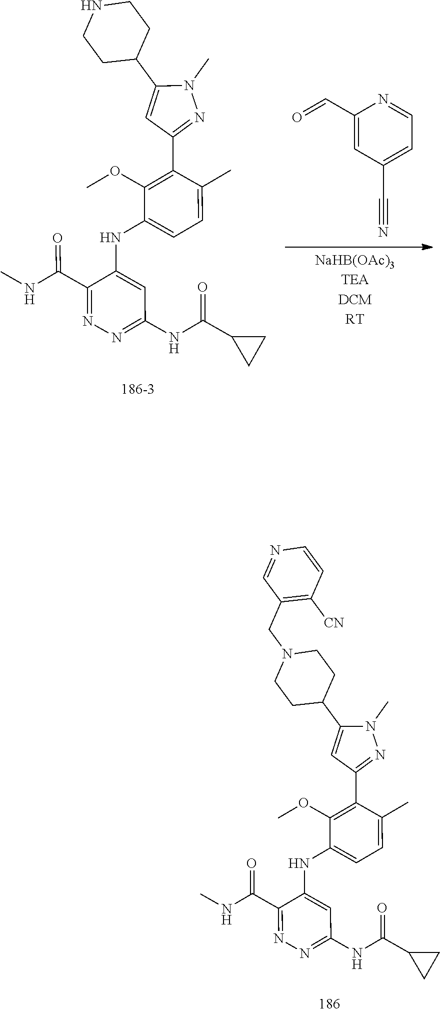 [SMILES: CNC(=O)c1nnc(NC(=O)C2CC2)cc1Nc1ccc(C)c(-c2cc(C3CCNCC3)n(C)n2)c1OC ; N#Cc1ccnc(C=O)c1 ; CNC(=O)c1nnc(NC(=O)C2CC2)cc1Nc1ccc(C)c(-c2cc(C3CCN(Cc4cnccc4C#N)CC3)n(C)n2)c1OC]

Step 1: To a solution of (5-(1-(tert-butoxycarbonyl)-1,2,3,6-tetrahydropyridin-4-yl)-1-methyl-1H-pyrazol-3-yl) boronic acid (900 mg, 1.0 equiv, 2.93 mmol) in MeOH (5.0 mL) was added Pd/C (93.5 mg, 0.3 equiv, 879 μmol). The reaction mixture was stirred at room temperature for 1 h under hydrogen atmosphere. The resulting mixture was filtered, and the filter cake was washed with MeOH. The filtrate was concentrated under reduced pressure. This resulted compound 186-1 (700 mg, 2.26 mmol, 77% yield as a white solid.

LC/MS (ES+) m/z 310.1 [M+H]+

Step 2: To a stirred solution of Intermediate I (660 mg, 1.0 equiv, 1.52 mmol), compound 186-1 (470 mg, 1.0 equiv, 1.52 mmol), K₂CO₃ (420 mg, 2.0 equiv, 3.04 mmol) in 1,4-dioxane (10 mL) and H₂O (2.0 mL) was added XPhos precatalyst (179 mg, 0.15 equiv, 228 μmol) and the resulting solution was stirred at 80° C. for 2 h under nitrogen atmosphere. The resulting mixture was concentrated under reduced pressure and was dissolved in DMF. The resulted solution was purified using C18 flash chromatography (gradient: MeCN/water) to provide compound 186-2 (800 mg, 1.29 mmol, 85% yield) as a yellow solid.

LC/MS (ES+) m/z=619.5 [M+H]+

Step 3: To a stirred solution compound 186-2 (750 mg, 1.0 equiv, 1.21 mmol) was added HCl in dioxane (10 mL, 1 equiv, 1.21 mmol) and the resulting solution was stirred at 25° C. for 1 hour. The resulting mixture was concentrated under reduced pressure to provide compound 186-3 (HCl salt, 600 mg, 1.08 mmol, 89.2%) as a light yellow oil.

m/z (ES+) [M+H]+=519.25

Step 4: To a solution of 186-3 (300 mg, 1.0 equiv, 540 μmol) and 2-formylisonicotinonitrile (143 mg, 2.0 equiv, 1.08 mmol) in DCM (2 mL) was added TEA (219 mg, 301 μL, 4.0 equiv, 2.16 mmol). After stirring at 25° C. for 0.5 h, sodium triacetoxyborohydride (115 mg, 1.0 equiv, 540 μmol) was added and the resulting mixture was stirred at room temperature for 0.5 h. The reaction was quenched with water and extracted with ethyl acetate. The organic layer was washed with brine, dried over Na₂SO₄ and evaporated. The residue was purified using HPLC(C18, gradient: MeCN, 10 mmol/L NH₄HCO₃+0.1% NH₃ in H₂O) to provide compound 186 (3, 2.10 g, 70%) as a yellow oil.

m/z (ES+) [M+H]+=635.35

¹H NMR (DMSO-d6, 400 MHZ) 11.28 (1H, s), 10.76 (1H, s), 9.12 (1H, q, J=4.8 Hz), 8.77 (1H, dd, J=5.0, 0.9 Hz), 8.07 (1H, s), 7.87 (1H, t, J=1.2 Hz), 7.75 (1H, dd, J=5.0, 1.6 Hz), 7.30 (1H, d, J=8.1 Hz), 7.09 (1H, d, J=8.3 Hz), 6.19 (1H, s), 3.83 (3H, s), 3.73 (2H, s), 3.41 (3H, s), 2.93 (2H, d, J=11.2 Hz), 2.85 (3H, d, J=4.8 Hz), 2.82-2.65 (1H, m), 2.24 (2H, t, J=11.4 Hz), 2.18 (3H, s), 2.09 (1H, p, J=6.2 Hz), 1.91 (2H, d, J=12.9 Hz), 1.74-1.60 (2H, m), 0.83 (4H, d, J=6.1 Hz).

### Example 187

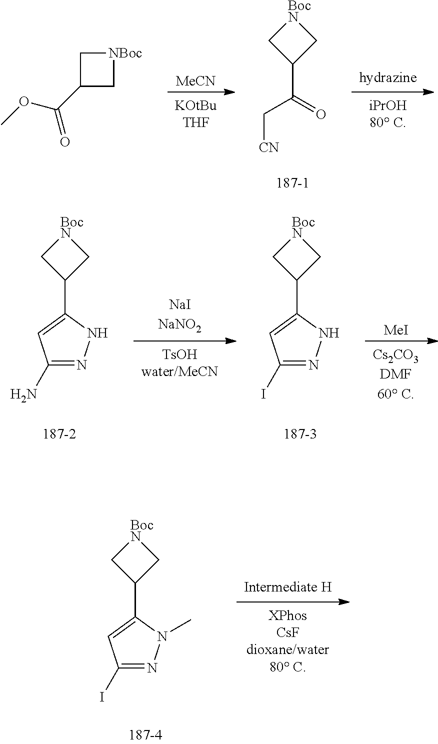

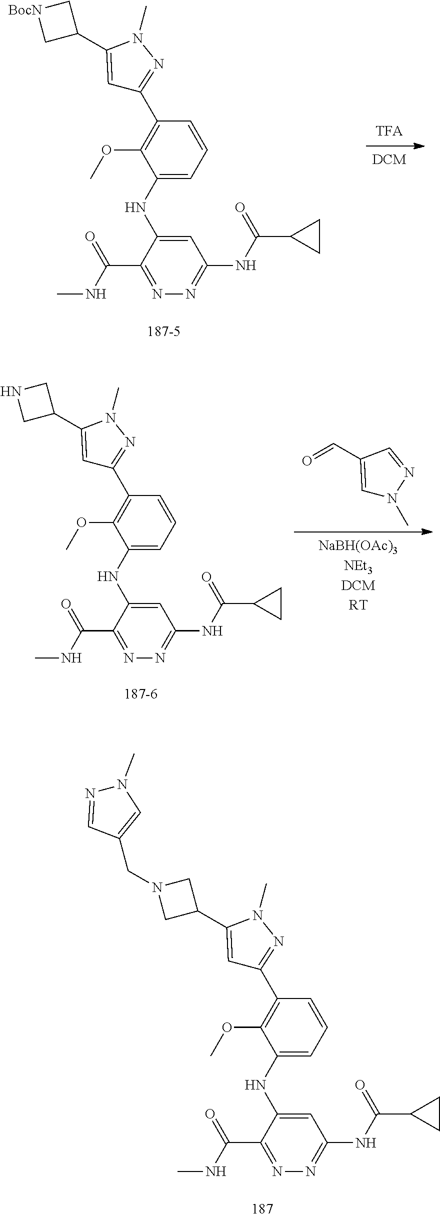

Step 1: To an ice-cooled solution of 1-(tert-butyl) 3-methyl azetidine-1,3-dicarboxylate (10 g, 1.0 equiv, 46 mmol) and MeCN (2.9 g, 3.6 mL, 1.5 equiv, 70 mmol) in tetrahydrofuran (250 mL) was added tBuOK (7.8 g, 8.8 mL, 1.5 equiv, 70 mmol) dropwise under the atmosphere of N₂. The resulting mixture was warmed to room temperature. After 1 h, the reaction mixture was poured into saturated aqueous ammonium chloride, and the resulting solution was extracted with ethyl acetate. The combined organics were washed with saturated aqueous sodium chloride solution, dried over anhydrous sodium sulfate, and concentrated in vacuo to give compound 187-1 (7 g, 0.03 mol, 70% yield) a colorless oil used that was used in next step directly.

m/z (ES+) [M−H]−=223.1

Step 2: To a solution of compound 187-1 (7 g, 1.0 equiv, 0.03 mol) in 2-propanol (140 mL) was added hydrazine monohydrate (28 mL). The mixture was heated to 80° C. After 16 h, the reaction mixture was concentrated in vacuo. The resulting residue was dissolved in dichloromethane, and the organic solution was washed sequentially with water and saturated aqueous sodium chloride solution. The organic layer was dried over anhydrous sodium sulfate, filtered, and concentrated in vacuo to afford compound 187-2 (7.1 g, 30 mmol, 100%) a colorless oil used in next step directly.

m/z (ES+) [M−H]−=237.1

Step 3: To an ice-cooled solution compound 187-2 (7.1 g, 1.0 equiv, 30 mmol) in MeCN (240 mL) and Water (48 mL) was added Ts-OH (17 g, 3.0 equiv, 89 mmol) and sodium nitrite (2.1 g, 1.2 mL, 1.0 equiv, 30 mmol). After 30 min, sodium iodide (8.9 g, 2.4 mL, 2.0 equiv, 60 mmol) was added, and the reaction mixture was warmed to room temperature. After 1 h, the reaction mixture was poured into water, and the resulting solution was extracted with ethyl acetate. The combined organic layer was washed with saturated aqueous sodium chloride solution, dried over anhydrous sodium sulfate, filtered, and concentrated. Purification by flash column chromatography to provide compound 187-3 (3.4 g, 9.7 mmol, 33% yield) as a yellow solid.

m/z (ES+) [M−H]−=347.90

Step 4: To a solution of compound 187-3 (3.4 g, 1 Eq, 9.7 mmol) in DMF (20 mL) was added iodomethane (4.1 g, 3.0 equiv, 29 mmol) and Cs₂CO₃ (6.3 g, 2.0 equiv, 19 mmol). The mixture was stirred at 50-60° C. for 10 h. Ethyl acetate was added to the reaction mixture, and the resulting suspension was filtered. The filtrate was concentrated in vacuo. Purification by flash column chromatography afforded compound 187-4 (1.5 g, 4.1 mmol, 42% yield) as a yellow oil.

m/z (ES+) [M+H]+=363.9

Step 5: A round bottomed flask was charged with compound 187-4 (150 mg, 1.0 equiv, 413 μmol), Intermediate H (193 mg, 1.0 equiv, 413 μmol), cesium fluoride (188 mg, 3.0 equiv, 1.24 mmol) and methanesulfonato (2-dicyclohexylphosphino-2′,4′,6′-tri-i-propyl-1,1′-biphenyl) (2′-methylamino-1,1′-biphenyl-2-yl) palladium (II) (71.2 mg, 0.2 equiv, 82.6 μmol). 1,4-Dioxane (3 mL) and water (0.6 mL) were added, and the solution was stirred at 80° C. for 12 hour under N₂. The reaction was quenched with water and extracted with ethyl acetate. The organic layer was washed with brine, dried over Na₂SO₄ and evaporated. Then the crude product was purified by prep-TLC (petroleum ether/ethyl acetate: 2:1) to provide compound 187-5 (50 mg, 87 μmol, 21% yield) as a white solid.

m/z (ES+) [M+H]+=577.3

Step 6: A round bottomed flask was charged with compound 187-5 (30 mg, 1.0 equiv, 52 μmol) in DCM (1 mL), after which TFA (1 mL) was added, and the solution was stirred at 25° C. for 1 hour. The reaction was quenched with NaHCO₃ and extracted with ethyl acetate. The organic layer was dried over Na₂SO₄ and evaporated to give compound 187-6 (25 mg, 42 μmol, 81% yield) as an colorless oil used in next step directly.

m/z (ES+) [M+H]+=477.2

Step 7: A mixture of compound 187-6 (20 mg, 1.0 equiv, 42 μmol), 1-methyl-1H-pyrazole-4-carbaldehyde (6.5 mg, 1.4 equiv, 59 μmol) and triethylamine (25 mg, 6.0 equiv, 0.25 mmol) were stirred in DCM (3 mL) for 20 min at 25° C. after which triacetoxyhydroborate (18 mg, 2.0 equiv, 84 μmol) slowly, and the mixture was stirred at 25° C. for 30 min. The reaction was quenched with water and extracted with DCM. The organic layer was washed with brine, dried over Na₂SO₄ and evaporated, and purified HPLC(C18, gradient: MeCN/NH₄HCO₃+0.1% NH₃·H₂O in water). Lyophilization yielded compound 187 (15 mg, 26 μmol, 63% yield) as a white amorphous solid.

m/z (ES+) [M+H]+=571.3

¹H NMR (400 MHZ, Chloroform-d) 11.20 (s, 1H), 8.67 (s, 1H), 8.29 (s, 1H), 8.18 (d, J=5.4 Hz, 1H), 7.69 (dd, J=7.9, 1.6 Hz, 1H), 7.50-7.39 (m, 3H), 7.23 (t, J=7.9 Hz, 1H), 6.76 (d, J=0.7 Hz, 1H), 3.92 (s, 5H), 3.80 (s, 4H), 3.71 (s, 3H), 3.66 (s, 2H), 3.26 (s, 2H), 3.08 (d, J=5.1 Hz, 3H), 1.68 (ddd, J=12.4, 7.9, 4.4 Hz, 1H), 1.18-1.10 (m, 2H), 1.02-0.91 (m, 2H).

TABLE 18
The following compounds were prepared using analogous procedures to
compound 187 in Example 187:

| | | LCMS m/z | |
|---|---|---|---|
| Compound | Structure | (ES+) | ¹H NMR |
| 202 | 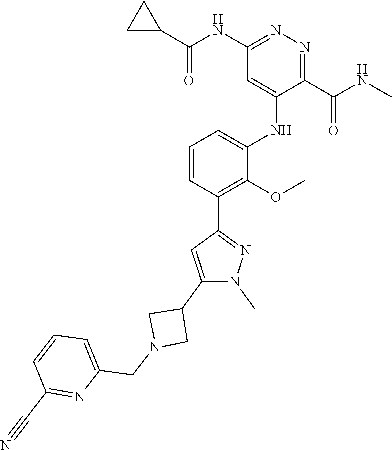 | 593.2 [M + H]+ | ¹H NMR (400 MHz, Chloroform-d) 11.81 (s, 1H), 8.81 (s, 1H), 8.29 (s, 1H), 8.17 (d, J = 5.3 Hz, 1H), 7.86 (t, J = 7.8 Hz, 1H), 7.72-7.62 (m, 3H), 7.44 (dd, J = 8.0, 1.6 Hz, 1H), 7.24 (t, J = 7.9 Hz, 1H), 6.82 (d, J = 0.6 Hz, 1H), 4.00 (d, J = 7.4 Hz, 2H), 3.92 (d, J = 21.9 Hz, 3H), 3.82 (s, 3H), 3.71 (s, 3H), 3.45 (d, J = 7.7 Hz, 2H), 3.08 (d, J = 5.1 Hz, 3H), 1.70 (tt, J = 8.1, 4.5 Hz, 1H), 1.18-1.10 (m, 2H), 0.95 (dq, J = 7.6, 4.1 Hz, 2H). |
| 203 | 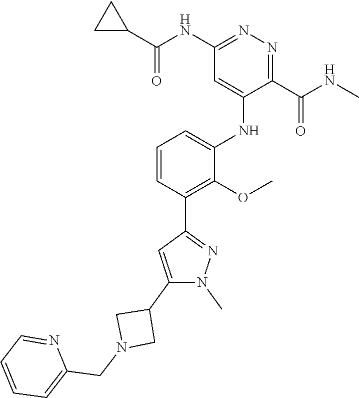 | 568.3 [M + H]+ | ¹H NMR (400 MHz, Chloroform-d) 11.16 (s, 1H), 8.67 (s, 1H), 8.64-8.57 (m, 1H), 8.27 (s, 1H), 8.18 (d, J = 5.4 Hz, 1H), 7.76-7.66 (m, 2H), 7.47- 7.37 (m, 2H), 7.23 (t, J = 7.7 Hz, 2H), 6.78 (d, J = 0.8 Hz, 1H), 4.02 (s, 2H), 3.92 (s, 3H), 3.81 (s, 3H), 3.71 (s, 3H), 3.44 (s, 2H), 3.08 (d, J = 5.1 Hz, 3H), 1.67 (dq, J = 7.9, 4.1 Hz, 1H), 1.18-1.10 (m, 2H), 0.95 (dq, J = 7.5, 4.1 Hz, 2H). |

### Example 190

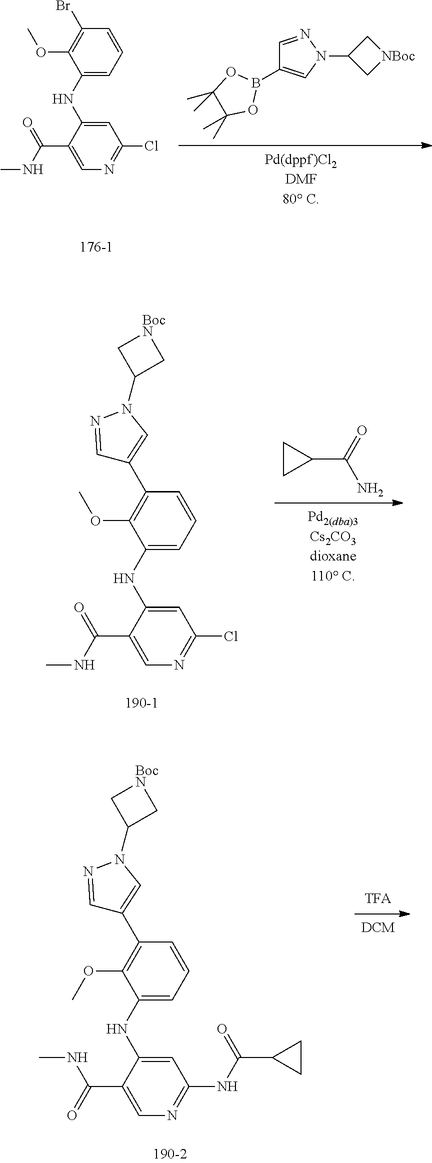 [SMILES: CNC(=O)c1cnc(Cl)cc1Nc1cccc(Br)c1OC ; CC(C)(C)OC(=O)N1CC(n2cc(B3OC(C)(C)C(C)(C)O3)cn2)C1 ; CNC(=O)c1cnc(Cl)cc1Nc1cccc(-c2cnn(C3CN(C(=O)OC(C)(C)C)C3)c2)c1OC ; NC(=O)C1CC1 ; CNC(=O)c1cnc(NC(=O)C2CC2)cc1Nc1cccc(-c2cnn(C3CN(C(=O)OC(C)(C)C)C3)c2)c1OC]

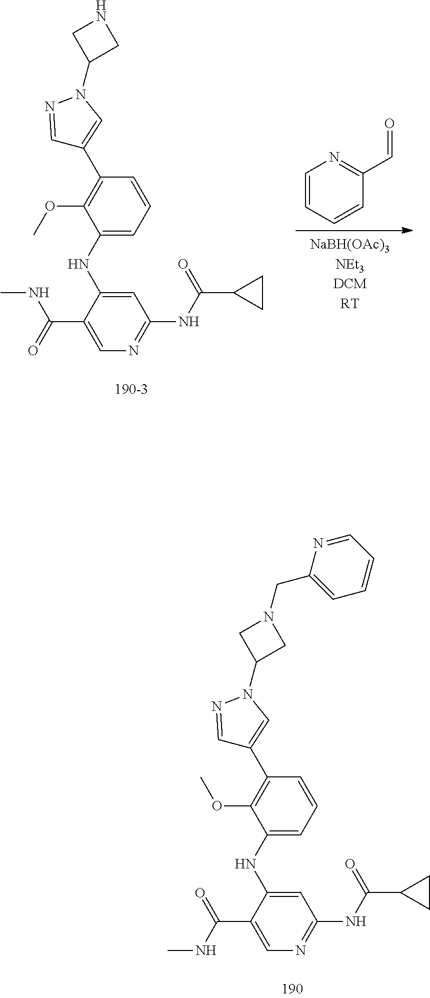 [SMILES: CNC(=O)c1cnc(NC(=O)C2CC2)cc1Nc1cccc(-c2cnn(C3CNC3)c2)c1OC ; O=Cc1ccccn1 ; CNC(=O)c1cnc(NC(=O)C2CC2)cc1Nc1cccc(-c2cnn(C3CN(Cc4ccccn4)C3)c2)c1OC]

Step 1: A solution of compound 176-1 (100 mg, 1.0 equiv, 270 μmol), tert-butyl 3-(4-(4,4,5,5-tetramethyl-1,3,2-dioxaborolan-2-yl)-1H-pyrazol-1-yl) azetidine-1-carboxylate (94.2 mg, 1.0 equiv, 270 μmol) and Pd(dppf)Cl₂, and K₂CO₃ (3.0 equiv) in a flask was stirred for 1.5 h at 80° C. after which the reaction was determined to be complete by LC/MS analysis. The crude reaction mixture was purified by flash silica gel chromatography to get to compound 190-1.

m/z (ES+) [M+H]+=513.1

Step 2: A 1,4-dioxane solution of compound 190-1 (1.0 equiv), cyclopropanecarboxamide (1.0 equiv) and Pd₂(dba)₃ (10 mol %), and Cs₂CO₃ in was stirred overnight at 110° C. until the reaction was determined to be complete by LC/MS analysis. The crude mixture was purified by flash silica gel chromatography to provide compound 190-2

m/z (ES+) [M+H]+=562.3

Step 3: A DCM solution of compound 190-2 (3 g, 1.0 equiv, 0.01 mol), TFA (1 g, 1.0 equiv, 0.01 mol) was stirred for overnight at room temperature until the reaction was determined to be complete by LC/MS analysis. The reaction mixture was concentrated to provide compound 190-3.

m/z (ES+) [M+H]+=462.3

Step 4: A solution of compound 190-3 (360 mg, 1.0 equiv, 780 μmol), picolinaldehyde (83.5 mg, 1.0 equiv, 780 μmol) and TEA (4.0 equiv) and sodium triacetoxyborohydride (2.0 equiv) was stirred for 1.5 h at room temperature until the reaction was determined to be complete by LC/MS analysis. The crude reaction mixture was purified by flash silica gel chromatography to provide compound 190 (300 mg, 543 μmol, 70% yield) as light yellow amorphous solid.

m/z (ES+) [M+H]+=552.6

¹H NMR (400 MHZ, DMSO-d6): Ä 10.75 (s, 1H), 10.68 (s, 1H), 8.61 (s, 1H), 8.51 (d, J=5.9 Hz, 2H), 8.37 (s, 1H), 8.04 (d, J=14.8 Hz, 2H), 7.82-7.73 (m, 1H), 7.45-7.36 (m, 2H), 7.32-7.23 (m, 2H), 7.16 (t, J=7.9 Hz, 1H), 5.12 (t, J=6.8 Hz, 1H), 3.86-3.77 (m, 4H), 3.59 (s, 4H), 3.59 (d, J=14.5 Hz, 1H), 2.80 (d, J=4.5 Hz, 3H), 2.02-1.94 (m, 1H), 0.78 (d, J=6.0 Hz, 4H).

### Example 192

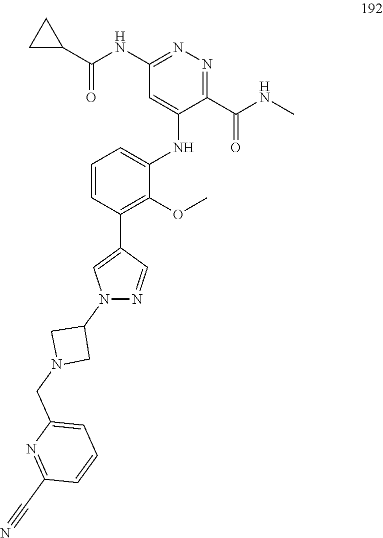 [SMILES: CNC(=O)c1nnc(NC(=O)C2CC2)cc1Nc1cccc(-c2cnn(C3CN(Cc4cccc(C#N)n4)C3)c2)c1OC]

Compound 192 was prepared in analogous fashion to compound 190 in Example 190 except that 6-formylpicolinonitrile was used in place of picolinaldehyde.

LC/MS (ES+) m/z=577.3 [M+H]+

¹H NMR (400 MHZ, DMSO-d6): |Ä 10.75 (s, 1H), 10.68 (s, 1H), 8.52 (s, 1H), 8.38 (s, 1H), 8.10-8.00 (m, 3H), 7.94 (d, J=7.5 Hz, 1H), 7.77 (d, J=8.0 Hz, 1H), 7.39 (d, J=7.9 Hz, 1H), 7.29 (d, J=7.8 Hz, 1H), 7.16 (t, J=7.9 Hz, 1H), 5.18-5.09 (m, 1H), 3.91 (s, 2H), 3.83 (t, J=7.4 Hz, 2H), 3.61 (d, J=11.7 Hz, 5H), 2.80 (d, J=4.4 Hz, 3H), 1.24 (s, 1H), 0.78 (d, J=6.0 Hz, 4H).

### Example 204

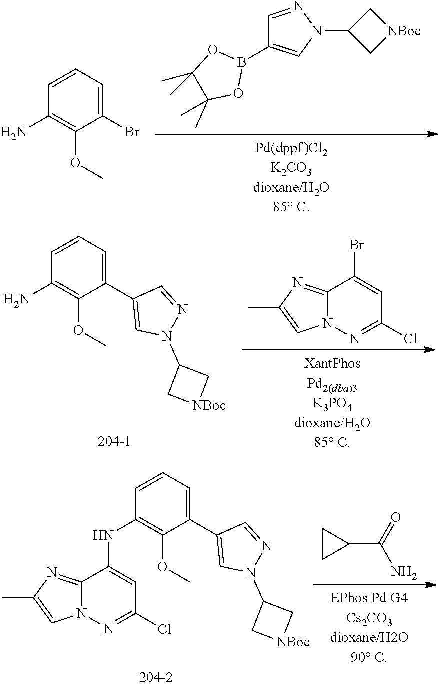

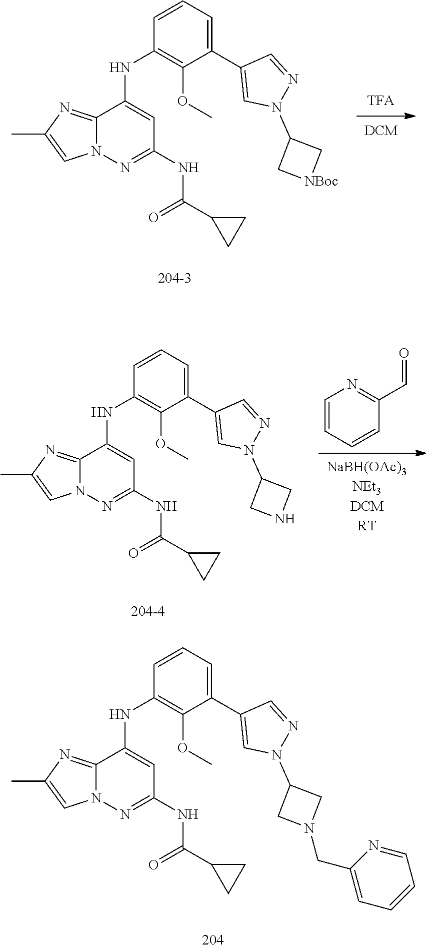 [SMILES: COc1c(Nc2cc(NC(=O)C3CC3)nn3cc(C)nc23)cccc1-c1cnn(C2CN(C(=O)OC(C)(C)C)C2)c1 ; COc1c(Nc2cc(NC(=O)C3CC3)nn3cc(C)nc23)cccc1-c1cnn(C2CNC2)c1 ; O=Cc1ccccn1 ; COc1c(Nc2cc(NC(=O)C3CC3)nn3cc(C)nc23)cccc1-c1cnn(C2CN(Cc3ccccn3)C2)c1]

Step 1: A round bottomed flask was charged with 3-bromo-2-methoxyaniline (1 g, 1.0 equiv, 5 mmol), tert-butyl 3-(4-(4,4,5,5-tetramethyl-1,3,2-dioxaborolan-2-yl)-1H-pyrazol-1-yl) azetidine-1-carboxylate (2 g, 1.2 equiv, 6 mmol), potassium carbonate (1 g, 2 equiv, 0.01 mol), 1,4-dioxane (20 mL), H₂O (4 mL) and a stirbar. Pd(dppf)Cl₂ (0.7 g, 0.2 equiv, 1 mmol) was added, and the solution was stirred at 85° C. for 2 hours. The resulting mixture was extracted with EtOAc. The combined organic layers were washed with water and then washed with brine, and dried over anhydrous Na₂SO₄. After filtration, the filtrate was concentrated under reduced pressure. The residue was purified by silica gel column chromatography to compound 204-1 (1.8 g, 5.2 mmol, 100% yield).

m/z (ES+) [M+H]+=289.15

Step 2: A round bottomed flask was charged compound 204-1 (1 g, 1.0 equiv, 3 mmol), 8-bromo-6-chloro-2-methylimidazo[1,2-b]pyridazine (0.9 g, 1.2 equiv, 3 mmol), tripotassium phosphate (1 g, 2.0 equiv, 6 mmol), 1,4-dioxane (15 mL), H₂O (3 mL) and a stirbar, xantphos (0.2 g, 0.1 equiv, 0.3 mmol), Pd₂(dba)₃ (0.3 g, 0.1 equiv, 0.3 mmol) was added. The solution was stirred at 85° C. for 1 hour until the reaction was determined to be complete by LC/MS analysis. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatography to afford compound 204-2 (800 mg, 1.57 mmol, 50% yield).

m/z (ES+) [M+H]+=510.20

Step 3: A round bottomed flask was charged with compound 204-2 (800 mg, 1.0 equiv, 1.57 mmol), cyclopropanecarboxamide (200 mg, 1.5 equiv, 2.35 mmol), Cs₂CO₃ (1.02 g, 2 equiv, 3.14 mmol), 1,4-dioxane (15 mL), H₂O (3 mL) and a stirbar. EPhos Pd G4 (0.2 equiv) was added, and the solution was stirred at 90° C. for 1 hour. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatography to afford compound 204-3 (420 mg, 752 μmol, 48% yield).

m/z (ES+) [M+H]+=559.35

Step 4: A round bottomed flask was charged with compound 204-3 (400 mg, 1.0 equiv, 716 μmol), DCM (5 mL) and a stirbar. TFA (163 mg, 2.0 equiv, 1.43 mmol) was added, and the solution was stirred at 25° C. for 1 hour. The mixture was then basified to pH>7 with NaHCO₃. The resulting mixture was extracted with EtOAc. The combined organic layers were washed with water and then washed with brine, dried over anhydrous Na₂SO₄. After filtration, the filtrate was concentrated under reduced pressure to afford compound 204-4 (300 mg, 654 μmol, 91 yield).

m/z (ES+) [M+H]+=459.3

Step 5: A mixture of compound 204-4 (300 mg, 1.0 equiv, 654 μmol), picolinaldehyde (105 mg, 1.5 equiv, 981 μmol) TEA (265 mg, 365 μL, 4 equiv, 2.62 mmol) were stirred in DCM (6 mL) for 20 minutes at RT, followed by the slow addition of sodium triacetoxyborohydride (277 mg, 2.0 equiv, 1.31 mmol) over 20 minutes. The resulting mixture was extracted with EtOAc. The combined organic layers were washed with water and then washed with brine, dried over anhydrous Na₂SO₄. After filtration, the filtrate was concentrated under reduced pressure. The crude product was purified by HPLC(C18, gradient MeCN/0.1% formic acid in water) to afford compound 204 (157.5 mg, 264.4 μmol, 40% yield) as an off-white amorphous solid.

m/z (ES+) [M+H]+=550.2

¹H NMR (400 MHZ, DMSO-d6) 10.69 (s, 1H), 8.72 (s, 1H), 8.55-8.50 (m, 1H), 8.36 (s, 1H), 8.14 (s, 1H), 8.05 (s, 1H), 7.79 (td, J=7.7, 1.9 Hz, 1H), 7.73 (d, J=1.0 Hz, 1H), 7.55 (dd, J=7.5, 2.0 Hz, 1H), 7.43 (d, J=7.8 Hz, 1H), 7.32-7.19 (m, 3H), 7.14 (s, 1H), 5.18 (p, J=6.9 Hz, 1H), 3.94 (d, J=14.3 Hz, 4H), 3.72 (s, 2H), 3.58 (s, 3H), 2.38 (s, 3H), 1.90 (p, J=6.8 Hz, 1H), 0.78-0.72 (m, 4H).

### Example 205

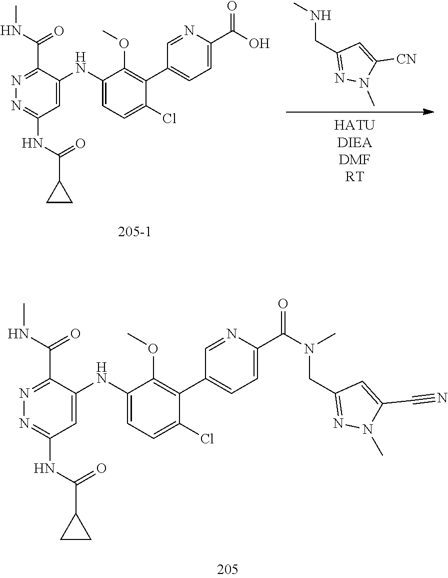 [SMILES: CNC(=O)c1nnc(NC(=O)C2CC2)cc1Nc1ccc(Cl)c(-c2ccc(C(=O)O)nc2)c1OC ; CNCc1cc(C#N)n(C)n1 ; CNC(=O)c1nnc(NC(=O)C2CC2)cc1Nc1ccc(Cl)c(-c2ccc(C(=O)N(C)Cc3cc(C#N)n(C)n3)nc2)c1OC]

Compound 205-1 was prepared in analogous fashion to Intermediate H except that 3-bromo-4-chloro-2-methoxyaniline was used in place of 3-bromo-2-methoxyaniline.

Compound 205-1 was then converted to compound 205 according to the following procedure: A solution of compound 205-1 (80 mg, 1.0 equiv, 0.16 mmol), and DIEA (3.0 equiv), HATU (2.0 equiv) was stirred for 15 min at room temperature. 1-Methyl-3-((methylamino)methyl)-1H-pyrazole-5-carbonitrile (24 mg, 1.0 equiv, 0.16 mmol) was added to the resulting mixture and stirred for additional 1 h at room temperature after which the reaction was quenched with water and extracted with ethyl acetate. The organic layer was washed with brine, dried over Na₂SO₄ and evaporated. The resulting crude material was purified using prep-HPLC which resulted in compound 205 (59.5 mg, 94.6 μmol, 59% yield) as an off-white amorphous solid.

m/z (ES+) [M+H]+=630.1

¹H NMR (400 MHZ, DMSO-d6): |Ä 11.39 (s, 1H), 10.96 (d, J=3.2 Hz, 1H), 9.19 (q, J=5.1 Hz, 1H), 8.64-8.58 (m, 1H), 8.17 (d, J=3.6 Hz, 1H), 8.00 (ddd, J=8.1, 4.1, 2.2 Hz, 1H), 7.75 (dd, J=8.0, 6.8 Hz, 1H), 7.58 (dd, J=8.7, 1.8 Hz, 1H), 7.50 (d, J=8.7 Hz, 1H), 7.11 (s, OH), 4.70 (s, 1H), 4.64 (s, 1H), 4.02 (s, 1H), 3.97 (s, 1H), 3.36 (d, J=5.0 Hz, 3H), 3.01 (d, J=1.7 Hz, 3H), 2.85 (d, J=4.8 Hz, 3H), 2.10 (td, J=7.0, 3.3 Hz, 1H), 0.86 (q, J=3.2, 2.6 Hz, 4H).

### Example 214

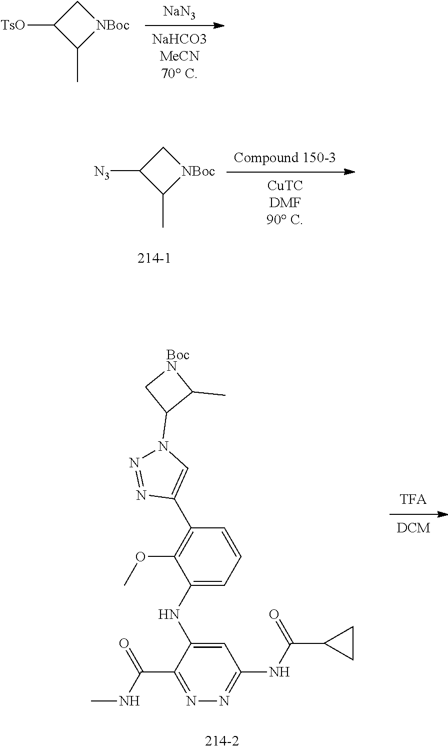 [SMILES: Cc1ccc(S(=O)(=O)OC2CN(C(=O)OC(C)(C)C)C2C)cc1 ; CC1C(N=[N+]=[N-])CN1C(=O)OC(C)(C)C ; CNC(=O)c1nnc(NC(=O)C2CC2)cc1Nc1cccc(-c2cn(C3CN(C(=O)OC(C)(C)C)C3C)nn2)c1OC ; "NaN3"]

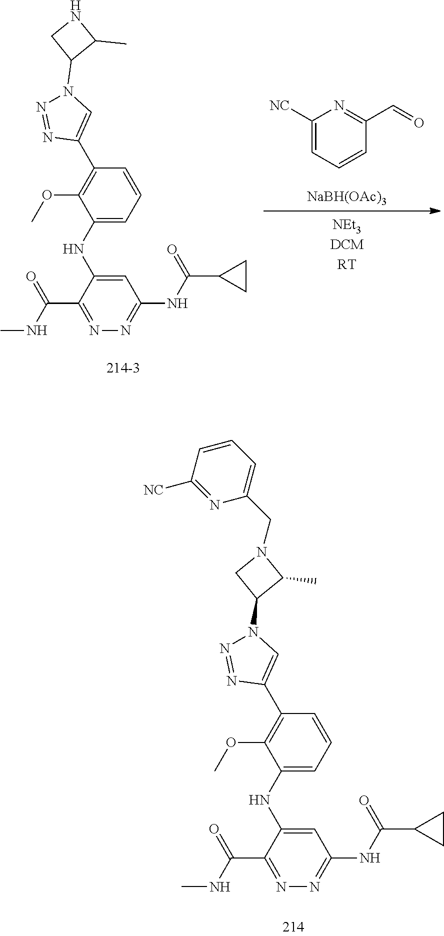 [SMILES: CNC(=O)c1nnc(NC(=O)C2CC2)cc1Nc1cccc(-c2cn(C3CNC3C)nn2)c1OC ; N#Cc1cccc(C=O)n1 ; CNC(=O)c1nnc(NC(=O)C2CC2)cc1Nc1cccc(-c2cn([C@H]3CN(Cc4cccc(C#N)n4)[C@@H]3C)nn2)c1OC ; "NaBH(OAc)3"]

Step 1: In a 40 mL vial was charged tert-butyl 2-methyl-3-(tosyloxy) azetidine-1-carboxylate (350 mg, 1.0 equiv, 1.03 mmol) and sodium azide (200 mg, 3.0 equiv, 3.08 mmol) and NaHCO₃ (2.0 equiv) in MeCN and the reaction was heated to 70° C. for 5 hrs until it was determined to be complete by LC/MS analysis. The reaction was then cooled to rt, diluted with MTBE, washed with water, after which the organics were dried and concentrated to provide compound 214-1 as a crude oil that was used directly in the next step.

Step 2: In a 2 dram vial was charged compound 150-3 (45 mg, 1.0 equiv, 0.12 mmol), compound 214-1 (0.21 g, 25% Wt, 2.0 equiv, 0.25 mmol), copper (I) thiophene-2-carboxylate (7.0 mg, 0.3 equiv, 37 μmol) after which DMF (1.2 mL) was added and the reaction was heated to 90° C. for 10 hrs. The reaction was then diluted with EtOAc and washed with water, brined, dried and concentrated. The crude material was purified by flash silica gel column chromatography (gradient: DCM/MeOH) to provide compound 214-2 as a white solid that was used directly in the next step.

Step 3: Compound 214-2 was deprotected under standard TFA/DCM conditions to provide compound 214-3 that was used directly in the next step.

Step 4: Compound 214-3 was dissolved in 0.2 mL after which aldehyde (4.0 equiv) and 10 eq of TEA (10 equiv) and the mixture was stirred at rt for 30 min after which NaBH(OAc)₃ (5.0 equiv) and stirred for 18 hrs. The reaction was then diluted with DCM and water. The organics were separated, dry loaded on silica and purified by flash silica gel chromatography (gradient: DCM/MeOH). The product fractions were combined and concentrated to collect compound 214 as a the racemic mixture of the trans diastereomer.

LC/MS (ES+) m/z=594.5 [M+H]+

¹H NMR (500 MHZ, DMSO) δ 11.30 (s, 1H), 10.98 (s, 1H), 9.16 (d, J=4.9 Hz, 1H), 8.62 (s, 1H), 8.11 (s, 1H), 8.04 (t, J=7.8 Hz, 1H), 7.94 (dd, J=7.6, 1.1 Hz, 1H), 7.91 (dd, J=7.9, 1.6 Hz, 1H), 7.80 (dd, J=8.1, 1.1 Hz, 1H), 7.43 (dd, J=8.0, 1.6 Hz, 1H), 7.30 (t, J=7.9 Hz, 1H), 5.74 (s, 2H), 4.97 (q, J=7.5 Hz, 1H), 4.04 (d, J=14.2 Hz, 1H), 3.90-3.84 (m, 2H), 3.80 (p, J=6.2 Hz, 1H), 3.65 (s, 3H), 3.51 (t, J=7.5 Hz, 1H), 2.86 (d, J=4.7 Hz, 3H), 2.06 (q, J=7.0, 6.2 Hz, 1H), 1.20 (d, J=6.1 Hz, 3H), 0.83-0.78 (m, 4H).

TABLE 19
The following compounds were prepared in analogous fashion to compound
214-3 in example 214 and separated using SFC to provide the enantiopure trans-azetidines:

| | | LCMS m/z | |
|---|---|---|---|
| Compound | Structure | (ES+) | ¹H NMR |
| 234 | 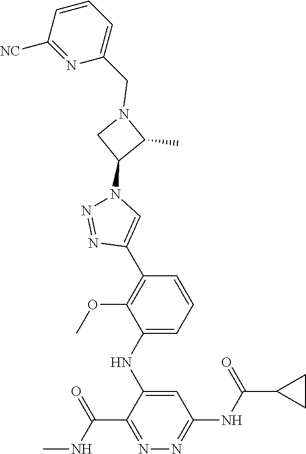 [SMILES: CNC(=O)c1nnc(NC(=O)C2CC2)cc1Nc1cccc(-c2cn([C@H]3CN(Cc4cccc(C#N)n4)[C@@H]3C)nn2)c1OC] | 594.5 [M + H]+ | ¹H NMR (500 MHz, DMSO) δ 11.31 (s, 0H), 10.98 (s, 0H), 9.03 (s, 2H), 8.18-8.06 (m, 3H), 7.78 (d, J = 7.8 Hz, 1H), 5.74 (s, 0H), 5.38 (s, 1H), 4.72 (s, 5H), 4.26 (s, 1H), 3.67 (s, 1H), 3.31 (s, 1H), 3.09 (qd, J = 7.3, 4.8 Hz, 9H), 2.86 (d, J = 4.9 Hz, 1H), 2.07 (ddd, J = 12.4, 7.7, 4.9 Hz, 0H), 1.51 (s, 1H), 1.39 (s, 2H), 1.17 (t, J = 7.3 Hz, 13H), 0.85- 0.76 (m, 2H). |
| 235 | 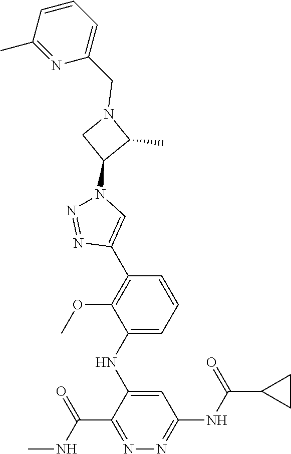 [SMILES: CNC(=O)c1nnc(NC(=O)C2CC2)cc1Nc1cccc(-c2cn([C@H]3CN(Cc4cccc(C)n4)[C@@H]3C)nn2)c1OC] | 583.5 [M + H]+ | ¹H NMR (500 MHz, DMSO) δ 11.31 (s, 1H), 11.12 (s, 3H), 10.98 (s, 1H), 9.17 (q, J = 4.8 Hz, 1H), 9.07 (s, 2H), 8.75 (s, 1H), 8.10 (s, 1H), 8.01-7.96 (m, 1H), 7.99-7.91 (m, 3H), 7.62-7.55 (m, 4H), 7.49 (d, J = 7.6 Hz, 2H), 7.45 (dd, J = 7.9, 1.7 Hz, 1H), 7.31 (t, J = 7.9 Hz, 1H), 5.74 (s, 3H), 5.44 (s, 1H), 5.39 (t, J = 5.9 Hz, 2H), 4.90 (s, 3H), 4.63 (s, 9H), 4.55 (s, 1H), 4.50 (d, J = 14.7 Hz, 3H), 4.27 (s, 2H), 3.67 (s, 3H), 3.31 (s, 6H), 3.09 (qd, J = 7.3, 4.8 Hz, 9H), 2.86 (d, J = 4.8 Hz, 3H), 2.46 (s, 2H), 2.30 (s, 1H), 2.06 (ddd, J = 9.9, 7.6, 4.8 Hz, 1H), 1.54 (s, 2H), 1.40 (s, 6H), 1.30 (d, J = 6.1 Hz, 1H), 1.17 (t, J = 7.3 Hz, 12H), 0.80 (ddt, J = 10.1, 8.4, 4.2 Hz, 4H). |
| 236 | 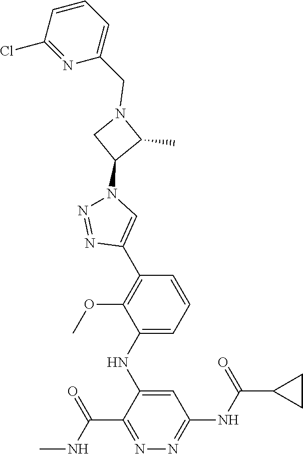 [SMILES: CNC(=O)c1nnc(NC(=O)C2CC2)cc1Nc1cccc(-c2cn([C@H]3CN(Cc4cccc(Cl)n4)[C@@H]3C)nn2)c1OC] | 603.4 [M + H]+ | ¹H NMR (500 MHz, DMSO) δ 11.31 (s, 1H), 11.12 (s, 3H), 10.98 (s, 1H), 9.17 (q, J = 4.8 Hz, 1H), 9.07 (s, 2H), 8.75 (s, 1H), 8.10 (s, 1H), 8.01-7.96 (m, 1H), 7.99-7.91 (m, 3H), 7.62-7.55 (m, 4H), 7.49 (d, J = 7.6 Hz, 2H), 7.45 (dd, J = 7.9, 1.7 Hz, 1H), 7.31 (t, J = 7.9 Hz, 1H), 5.74 (s, 3H), 5.44 (s, 1H), 5.39 (t, J = 5.9 Hz, 2H), 4.90 (s, 3H), 4.63 (s, 9H), 4.55 (s, 1H), 4.50 (d, J = 14.7 Hz, 3H), 4.27 (s, 2H), 3.67 (s, 3H), 3.31 (s, 6H), 3.09 (qd, J = 7.3, 4.8 Hz, 9H), 2.86 (d, J = 4.8 Hz, 3H), 2.46 (s, 2H), 2.30 (s, 1H), 2.06 (ddd, J = 9.9, 7.6, 4.8 Hz, 1H), 1.54 (s, 2H), 1.40 (s, 6H), 1.30 (d, J = 6.1 Hz, 1H), 1.17 (t, J = 7.3 Hz, 12H), 0.80 (ddt, J = 10.1, 8.4, 4.2 Hz, 4H). |
| 237 | 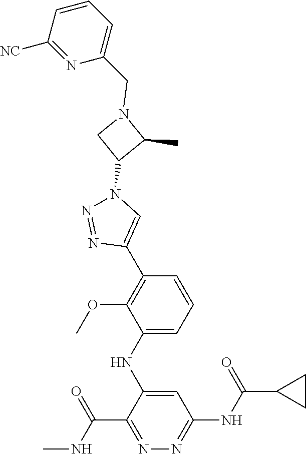 | 594.5 [M + H]+ | ¹H NMR (500 MHz, DMSO) δ 11.31 (s, 0H), 10.98 (s, 0H), 9.03 (s, 2H), 8.18-8.06 (m, 3H), 7.78 (d, J = 7.8 Hz, 1H), 5.74 (s, 0H), 5.38 (s, 1H), 4.72 (s, 5H), 4.26 (s, 1H), 3.67 (s, 1H), 3.31 (s, 1H), 3.09 (qd, J = 7.3, 4.8 Hz, 9H), 2.86 (d, J = 4.9 Hz, 1H), 2.07 (ddd, J = 12.4, 7.7, 4.9 Hz, 0H), 1.51 (s, 1H), 1.39 (s, 2H), 1.17 (t, J = 7.3 Hz, 13H), 0.85- 0.76 (m, 2H). |
| 238 | 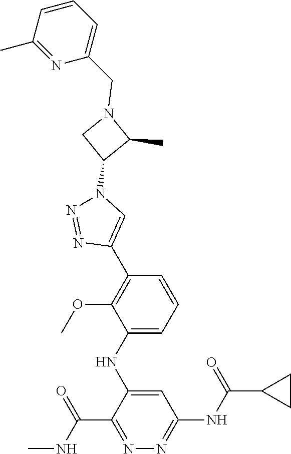 [SMILES: CNC(=O)c1nnc(NC(=O)C2CC2)cc1Nc1cccc(-c2cn([C@@H]3CN(Cc4cccc(C)n4)[C@H]3C)nn2)c1OC] | 583.5 [M + H]+ | ¹H NMR (500 MHz, DMSO) δ 11.31 (s, 1H), 11.12 (s, 3H), 10.98 (s, 1H), 9.17 (q, J = 4.8 Hz, 1H), 9.07 (s, 2H), 8.75 (s, 1H), 8.10 (s, 1H), 8.01-7.96 (m, 1H), 7.99-7.91 (m, 3H), 7.62-7.55 (m, 4H), 7.49 (d, J = 7.6 Hz, 2H), 7.45 (dd, J = 7.9, 1.7 Hz, 1H), 7.31 (t, J = 7.9 Hz, 1H), 5.74 (s, 3H), 5.44 (s, 1H), 5.39 (t, J = 5.9 Hz, 2H), 4.90 (s, 3H), 4.63 (s, 9H), 4.55 (s, 1H), 4.50 (d, J = 14.7 Hz, 3H), 4.27 (s, 2H), 3.67 (s, 3H), 3.31 (s, 6H), 3.09 (qd, J = 7.3, 4.8 Hz, 9H), 2.86 (d, J = 4.8 Hz, 3H), 2.46 (s, 2H), 2.30 (s, 1H), 2.06 (ddd, J = 9.9, 7.6, 4.8 Hz, 1H), 1.54 (s, 2H), 1.40 (s, 6H), 1.30 (d, J = 6.1 Hz, 1H), 1.17 (t, J = 7.3 Hz, 12H), 0.80 (ddt, J = 10.1, 8.4, 4.2 Hz, 4H). |
| 239 | 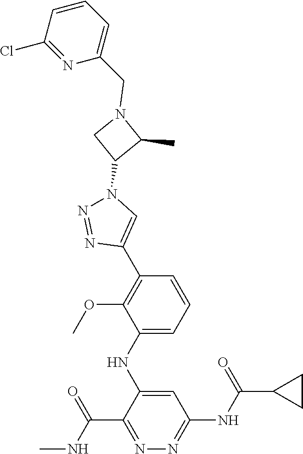 | 603.4 [M + H]+ | ¹H NMR (500 MHz, DMSO) δ 11.31 (s, 1H), 11.12 (s, 3H), 10.98 (s, 1H), 9.17 (q, J = 4.8 Hz, 1H), 9.07 (s, 2H), 8.75 (s, 1H), 8.10 (s, 1H), 8.01-7.96 (m, 1H), 7.99-7.91 (m, 3H), 7.62-7.55 (m, 4H), 7.49 (d, J = 7.6 Hz, 2H), 7.45 (dd, J = 7.9, 1.7 Hz, 1H), 7.31 (t, J = 7.9 Hz, 1H), 5.74 (s, 3H), 5.44 (s, 1H), 5.39 (t, J = 5.9 Hz, 2H), 4.90 (s, 3H), 4.63 (s, 9H), 4.55 (s, 1H), 4.50 (d, J = 14.7 Hz, 3H) 4.27 (s, 2H), 3.67 (s, 3H), 3.31 (s, 6H), 3.09 (qd, J = 7.3, 8.4 Hz, 9H), 2.86 (d, J = 4.8 Hz, 3H), 2.46 (s, 2H), 2.30 (s, 1H), 2.06 (ddd, J = 9.9, 7.6, 4.8 Hz, 1H), 1.54 (s, 2H), 1.40 (s, 6H), 1.30 (d, J = 6.1 Hz, 1H), 1.17 (t, J = 7.3 Hz, 12H), 0.80 (ddt, J = 10.1, 8.4, 4.2 Hz, 4H). |

### Example 217

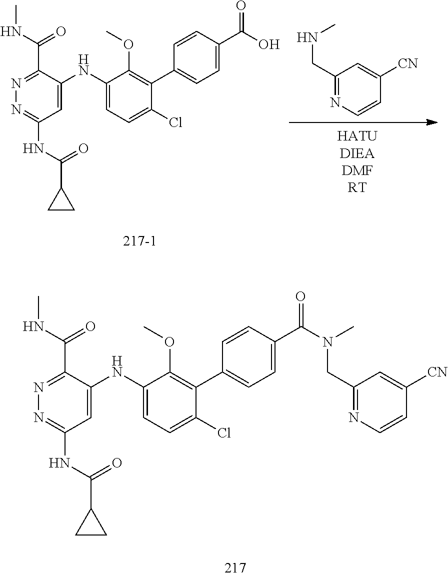 [SMILES: CNC(=O)c1nnc(NC(=O)C2CC2)cc1Nc1ccc(Cl)c(-c2ccc(C(=O)O)cc2)c1OC ; CNCc1cc(C#N)ccn1 ; CNC(=O)c1nnc(NC(=O)C2CC2)cc1Nc1ccc(Cl)c(-c2ccc(C(=O)N(C)Cc3cc(C#N)ccn3)cc2)c1OC]

Compound 217-1 was synthesized using analogous procedures in Example 11 except that 3-bromo-4-chloro-2-methoxyaniline was used in place of 3-bromo-2-methoxyaniline. Compound 217-1 was then converted into 217 using the analogous coupling conditions in example 205.

LC/MS (ES+) m/z=625.3 [M+H}+

¹H NMR (400 MHZ, DMSO-d6) 11.37 (s, 1H), 10.94 (s, 1H), 9.16 (s, 1H), 8.84 (s, 1H), 8.17 (s, 1H), 7.93-7.73 (m, 2H), 7.64 (s, 1H), 7.57-7.32 (m, 5H), 4.77 (d, J=67.7 Hz, 2H), 3.28 (s, 3H), 3.03 (d, J=29.0 Hz, 3H), 2.84 (d, J=4.8 Hz, 3H), 2.10 (s, 1H), 0.86 (d, J=7.4 Hz, 4H).

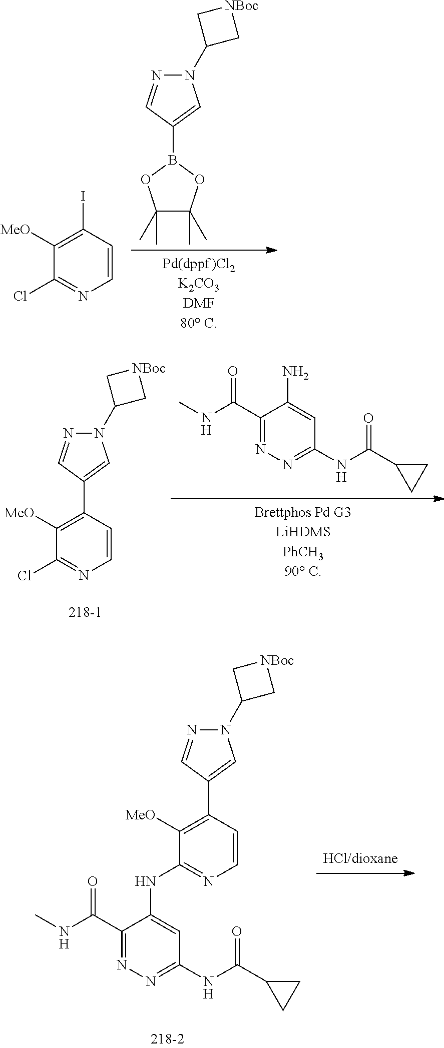

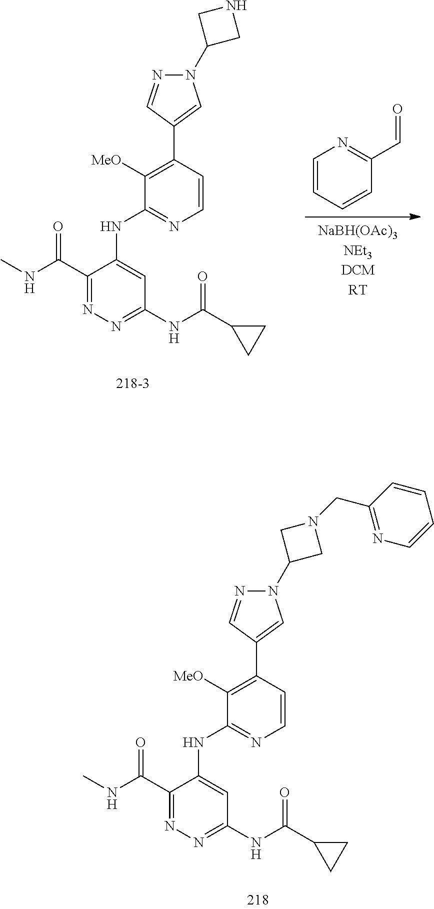 [SMILES: CNC(=O)c1nnc(NC(=O)C2CC2)cc1Nc1nccc(-c2cnn(C3CNC3)c2)c1OC ; O=Cc1ccccn1 ; CNC(=O)c1nnc(NC(=O)C2CC2)cc1Nc1nccc(-c2cnn(C3CN(Cc4ccccn4)C3)c2)c1OC]

Step 1: A solution of tert-butyl 3-(4-(4,4,5,5-tetramethyl-1,3,2-dioxaborolan-2-yl)-1H-pyrazol-1-yl) azetidine-1-carboxylate (622 mg, 1.0 equiv, 1.78 mmol), 2-chloro-4-iodo-3-methoxypyridine (480 mg, 1.0 equiv, 1.78 mmol) and Pd(dppf)Cl₂, K₂CO₃ (3.0 equiv) was stirred for 1.5 h at 80° C. until the reaction was determined to be complete by LC/MS analysis. The reaction was then purified using flash silica gel chromatography to provide compound 218-1 that was used directly in the next step.

m/z (ES+) [M+H]+=365.1

Step 2: A toluene (3 mL) solution of compound 218-1 (150 mg, 1.0 equiv, 411 μmol), 4-amino-6-(cyclopropanecarboxamido)-N-methylpyridazine-3-carboxamide (116 mg, 1.2 equiv, 493 μmol) followed by the addition of Brettphos Pd G3, and LiHMDS after which the reaction was stirred for 1 h at 90° C. The reaction was determined to be complete by LC/MS analysis. The reaction was quenched with water and extracted with ethyl acetate. The organic layer was washed with brine, dried over Na₂SO₄ and evaporated. The resulting crude material was purified using flash silica gel chromatography to provide compound 218-2.

m/z (ES+) [M+H]+=564.3

Step 3: A DCM (2 mL) solution of compound 218-2 (1.0 equiv) and 4NCl in 1,4-dioxane was stirred for 1.5 h at room temperature to remove the Boc group. The reaction was quenched with water and extracted with DCM. The organic layer was washed with brine, dried over Na₂SO₄ and evaporated to get the crude product 218-3 which can be used directly.

m/z (ES+) [M+H]+=464.2

Step 4: A solution of compound 218-3 (80 mg, 1.0 equiv, 0.17 mmol), picolinaldehyde (18 mg, 1.0 equiv, 0.17 mmol) followed by TEA (4.0 equiv), and NaBH(OAc)₃ (3.0 equiv) and was stirred for 1.5 h at room temperature until the reaction was determined to be complete by LC/MS analysis. The crude mixture was purified by flash silica gel chromatography to provide compound 218 as off-white amorphous solid.

m/z (ES+) [M+H]+=555.3

¹H NMR (400 MHZ, DMSO-d6): |Ä 12.34 (s, 1H), 11.32 (s, 1H), 9.85 (s, 1H), 9.27 (q, J=4.7 Hz, 1H), 8.60 (s, 1H), 8.51 (dd, J=4.9, 1.8 Hz, 1H), 8.19 (s, 1H), 8.05 (d, J=5.4 Hz, 1H), 7.78 (td, J=7.7, 1.9 Hz, 1H), 7.42 (d, J=7.8 Hz, 1H), 7.33 (d, J=5.3 Hz, 1H), 7.27 (dd, J=7.6, 4.8 Hz, 1H), 5.16 (p, J=6.9 Hz, 1H), 3.88° C. 3.79 (m, 4H), 3.76 (s, 3H), 3.61 (td, J=6.6, 1.6 Hz, 2H), 2.89 (d, J=4.8 Hz, 3H), 2.13 (td, J=7.5, 3.8 Hz, 1H), 0.94 \ddot{ }0.82 (m, 4H).

### Example 219

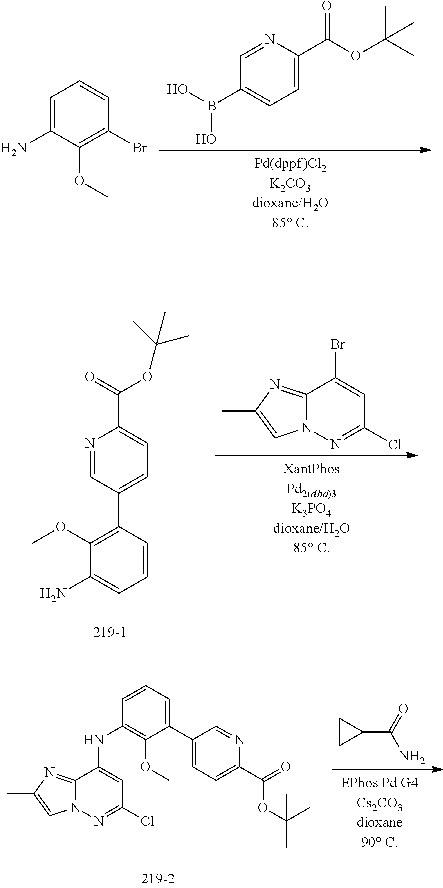

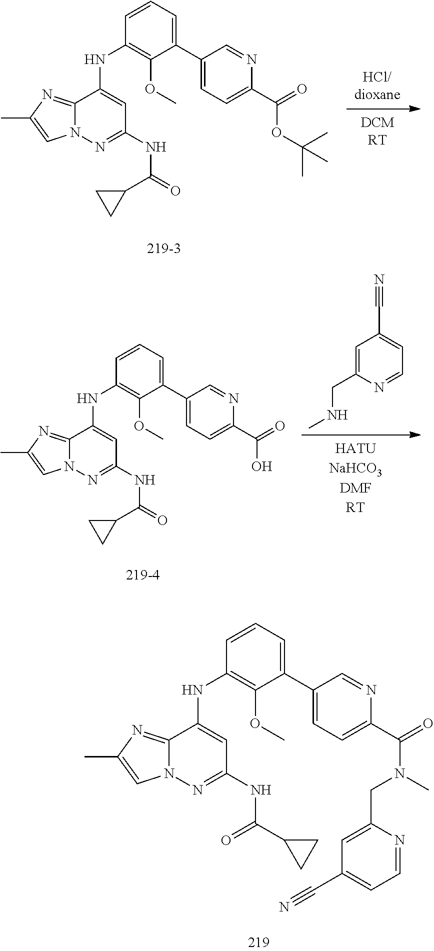

Step 1: A round bottomed flask was charged with 3-bromo-2-methoxyaniline (2 g, 1.0 equiv, 0.01 mol), (6-(tert-butoxycarbonyl)pyridin-3-yl) boronic acid (3 g, 1.2 equiv, 0.01 mol), 1,4-dioxane (30 mL), H₂O (6 mL) and a stirbar after which K₂CO₃ (3 g, 2 Eq, 0.02 mol), PdCl₂(dppf) (1 g, 0.2 equiv, 2 mmol) was added, and the solution was stirred at 85° C. for 2 hour. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatography to afford compound 219-1 (2.3 g, 7.7 mmol, 80% yield).

m/z (ES+) [M+H]+=301.1

Step 2: A round bottomed flask was charged with compound 219-1 (1 g, 1 equiv, 3 mmol), 8-bromo-6-chloro-2-methylimidazo[1,2-b]pyridazine (1 g, 1.2 equiv, 4 mmol), tripotassium phosphate (1 g, 2 equiv, 7 mmol), 1,4-dioxane (15 mL), H₂O (3 mL) and a stirbar after which xantphos (0.2 g, 0.1 equiv, 0.3 mmol), Pd₂(dba)₃ (0.3 g, 0.1 equiv, 0.3 mmol) was added, and the solution was stirred at 85° C. for 1 hour. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatograph to afford compound 219-2 (1.3 g, 2.8 mmol, 80% yield).

m/z (ES+) [M+H]+=466.1

Step 3: A round bottomed flask was charged with compound 219-2 (1.3 g, 1.0 equiv, 2.8 mmol), cyclopropanecarboxamide (0.36 g, 1.5 equiv, 4.2 mmol), Cs₂CO₃ (1.8 g, 2.0 equiv, 5.6 mmol), 1,4-dioxane (20 mL), H₂O (4 mL) and a stirbar, after which EPhos Pd G4 (0.2 equiv) was added, and the solution was stirred at 90° C. for 1 hour. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatography to afford compound 219-3 (800 mg, 1.55 mmol, 56% yield).

m/z (ES+) [M+H]+=515.3

Step 4: A round bottomed flask was charged with compound 219-3 (800 mg, 1.0 equiv, 1.55 mmol), DCM (3 mL) and a stirbar. 4M HCl/Dioxane (5 mL) was added, and the solution was stirred at 25° C. for 2 hours. The resulting mixture was extracted with EtOAc. The combined organic layers were washed with water and then washed with brine, dried over anhydrous Na₂SO₄. After filtration, the filtrate was concentrated under reduced pressure to afford compound 219-4 (700 mg, 1.53 mmol, 98% yield).

m/z (ES+) [M+H]+=459.3

Step 5: A round bottomed flask was charged with compound 219-4 (120 mg, 1.5 equiv, 818 μmol), Intermediate N (183 mg, 4.0 equiv, 2.18 mmol), DMF (10 mL) and a stirbar. HATU (415 mg, 2.0 equiv, 1.09 mmol) was added, and the solution was stirred at 25° C. for 2 hours. The resulting mixture was extracted with EtOAc. The combined organic layers were washed with water and then washed with brine, dried over anhydrous Na₂SO₄. After filtration, the filtrate was concentrated under reduced pressure. The crude product was purified by Prep-HPLC with the following conditions (Column: Xselect CSH C18 OBD, gradient MeCN/0.1% formic acid in water) to afford compound 219 (119.6 mg, 201 μmol, 36.9%, 98.8% Purity) as a orange amorphous solid.

m/z (ES+) [M+H]+=588.2

¹H NMR (400 MHZ, Methanol-d4) |Ä 8.96-8.71 (m, 2H), 8.23 (ddd, J=37.2, 8.0, 2.1 Hz, 1H), 7.88-7.75 (m, 2H), 7.71-7.58 (m, 4H), 7.43-7.27 (m, 2H), 4.97 (d, J=2.9 Hz, 2H), 3.47 (d, J=21.5 Hz, 3H), 3.20 (d, J=17.3 Hz, 3H), 2.53-2.43 (m, 3H), 1.86 (td, J=7.9, 4.0 Hz, 1H), 1.05-0.84 (m, 4H).

### Example 220

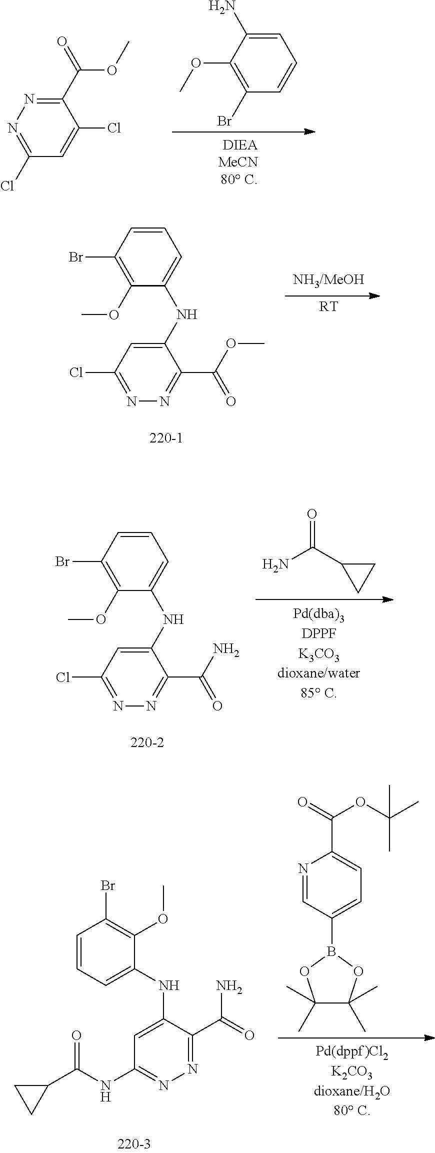

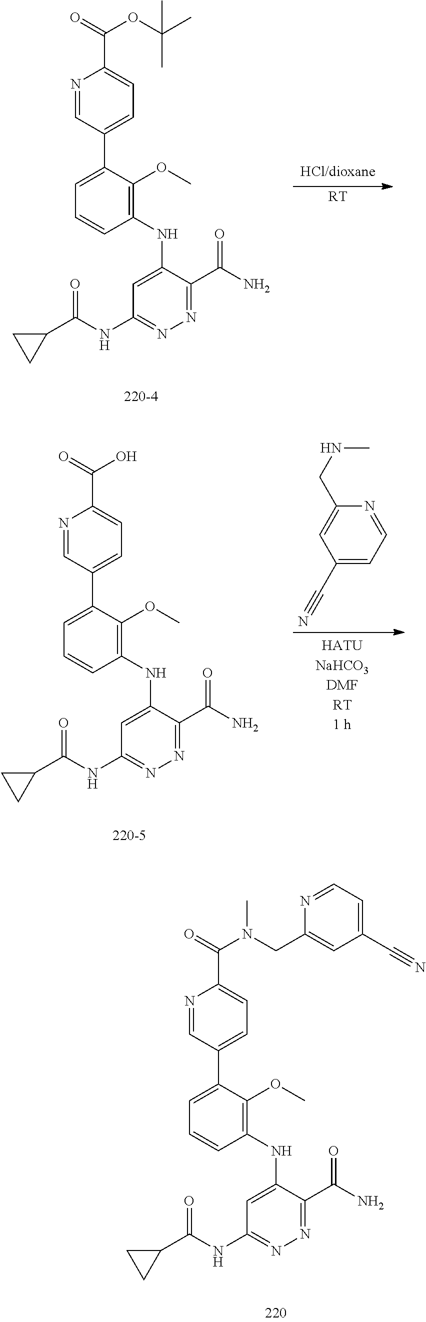 [SMILES: COc1c(Nc2cc(NC(=O)C3CC3)nnc2C(N)=O)cccc1-c1ccc(C(=O)OC(C)(C)C)nc1 ; "RT" ; COc1c(Nc2cc(NC(=O)C3CC3)nnc2C(N)=O)cccc1-c1ccc(C(=O)O)nc1 ; CNCc1cc(C#N)ccn1 ; COc1c(Nc2cc(NC(=O)C3CC3)nnc2C(N)=O)cccc1-c1ccc(C(=O)N(C)Cc2cc(C#N)ccn2)nc1]

Step 1: A round bottomed flask was charged with 3-bromo-2-methoxyaniline (5 g, 1.0 equiv, 0.02 mol)methyl 4,6-dichloropyridazine-3-carboxylate (5 g, 1.0 equiv, 0.02 mol), diisopropylethylamine (0.01 kg, 0.01 L, 3.0 equiv 0.07 mol) and a stir bar. MeCN (5 mL) was added, and the solution was stirred at 85° C. for 12 hour until the reaction was determined to be complete by LC/MS analysis. The reaction mixture was diluted with water (200 mL), and the aqueous phase was extracted with ethyl acetate (200 mL) three times. The combined organic layers were washed with saturated NaCl, dried over sodium sulfate, filtered, and concentrated in vacuo. The resulted solution was purified using C18 flash chromatography (gradient: MeCN/water). The reaction was concentrated in vacuo resulted in compound 220-1 as a off-white solid.

m/z (ES+) [M+H]+=373.85

Step 2: A round bottomed flask was charged with compound 220-1 (2 g, 1 equiv, 5 mmol), ammonia (0.5 g, 0.5 mL, 5 equiv, 0.03 mol) and a stir bar. MeOH (6 mL) was added, and the solution was stirred at 25° C. for 2 hours after which the reaction was determined to be complete by LC/MS analysis. The reaction mixture was diluted with water (100 mL), and the aqueous phase was extracted with ethyl acetate (300 mL) three times. The combined organic layers were washed with saturated NaCl, dried over sodium sulfate, filtered, and concentrated in vacuo. The resulted solution was purified using C18 flash chromatography (gradient: MeCN/water). Concentration in vacuo resulted in compound 220-2 as a off-white solid.

m/z (ES+) [M+H]+=358.85

Step 3: A round bottomed flask was charged with compound 220-2 (1 g, 1.0 equiv, 3 mmol) dppf (0.2 g, 0.1 equiv, 0.3 mmol) cyclopropanecarboxamide (0.6 g, 2.4 equiv, 7 mmol) Pd₂(dba)₃ (0.1 g, 0.05 equiv, 0.1 mmol) and a stir bar. 1,4-Dioxane (5 mL) and H₂O (1 mL) were added, and the solution was stirred at 85° C. for 12 hrs. The reaction mixture was diluted with water (100 mL), and the aqueous phase was extracted with ethyl acetate (100 mL) three times. The combined organic layers were washed with saturated NaCl, dried over sodium sulfate, filtered, and concentrated in vacuo. The resulted solution was purified using C18 flash chromatography (gradient: MeCN/water). Concentration in vacuo resulted in compound 220-3 as a off-white solid.

m/z (ES+) [M+H]+=408.1

Step 4: A round bottomed flask was charged with compound 220-3 (500 mg, 1.0 equiv, 1.23 mmol), PdCl₂ (dppf)-CH₂Cl₂ adduct (201 mg, 0.2 equiv, 246 μmol), (6-(tert-butoxycarbonyl)pyridin-3-yl) boronic acid (329 mg, 1.2 equiv, 1.48 mmol), and K₂CO₃ (510 mg, 3.0 equiv, 3.69 mmol) and a stir bar. 1,4-Dioxane (5 mL) and H₂O (1 mL) was added, and the solution was stirred at 85° C. for 2 hour until the reaction was determine to be complete by LC/MS analysis. The reaction mixture was diluted with water (50 mL), and the aqueous phase was extracted with ethyl acetate (30 mL) three times. The combined organic layers were washed with saturated NaCl, dried over sodium sulfate, filtered, and concentrated in vacuo. The resulting solution was purified using C18 flash chromatography (gradient: MeCN/water). Concentration in vacuo resulted compound 220-4 as a off-white solid.

m/z (ES+) [M+H]+=505.1

Step 5: A round bottomed flask was charged with compound 220-4 (300 mg, 1.0 equiv, 595 μmol), aqueous HCl (5.0 equiv, 2.97 mmol) and a stir bar. 1,4-Dioxane (2 mL) was added, and the solution was stirred at 25° C. for 2 hour after the reaction was determined to be complete by LC/MS analysis. The reaction mixture was diluted with water (50 mL), and the aqueous phase was extracted with ethyl acetate (30 mL) three times. The combined organic layers were washed with saturated NaCl dried over sodium sulfate, filtered, and concentrated in vacuo. The resulted solution was purified using C18 flash chromatography (gradient: MeCN/water). Concentration in vacuo resulted in compound 220-5 as a off-white solid.

m/z (ES+) [M+H]+=449.3

Step 6: A round bottomed flask was charged with compound 220-5 (200 mg, 1.0 equiv, 446 μmol), HATU (254 mg, 1.5 equiv, 669 μmol), 2-((methylamino)methyl) isonicotinonitrile (65.6 mg, 1.0 equiv, 446 μmol), NaHCO₃ (4.0 equiv, 1.78 mmol) and a stir bar. DMF was added, and the solution was stirred at 25° C. for 2 hour after which the reaction was determined to be complete by LC/MS analysis. The reaction mixture was diluted with water (100 mL), and the aqueous phase was extracted with ethyl acetate (100 mL) three times. The combined organic layers were washed with saturated NaCl, dried over sodium sulfate, filtered, and concentrated in vacuo. The resulted solution was purified using C18 flash chromatography with the following conditions (gradient: MeCN/water). Concentration in vacuo resulted in compound 220 as a off-white solid.

m/z (ES+) [M+H]+=578.2

¹H NMR (400 MHZ, DMSO-d6) 11.34 (d, J=5.3 Hz, 1H), 11.03 (d, J=12.9 Hz, 1H), 8.88-8.78 (m, 2H), 8.54 (d, J=4.5 Hz, 1H), 8.22-7.89 (m, 2H) 7.85-7.68 (m, 4H), 7.54 (ddd, J=12.1, 7.2, 2.3 Hz, 1H), 7.40-7.25 (m, 2H), 4.89 (d, J=10.3 Hz, 2H), 3.41-3.14 (m, 3H), 3.14-3.05 (m, 3H), 2.14-2.04 (m, 1H), 0.83 (m, 4H).

### Example 223

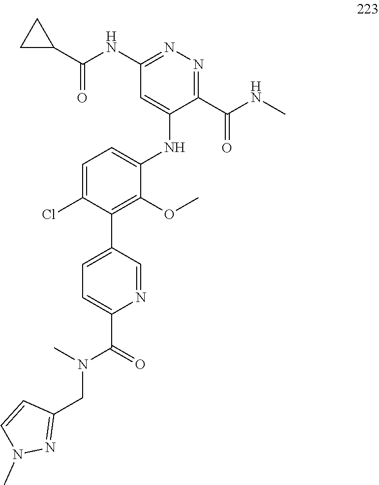 [SMILES: CNC(=O)c1nnc(NC(=O)C2CC2)cc1Nc1ccc(Cl)c(-c2ccc(C(=O)N(C)Cc3ccn(C)n3)nc2)c1OC]

Compound 223 was prepared in analogous fashion to compound 205 in Example 205 except that 1-(1,3-dimethyl-1H-pyrazol-4-yl)-N-methylmethanamine was used in place of N-methyl-1-(1-methyl-1H-pyrazol-3-yl) methanamine.

m/z (ES+) [M+H]+=603.2

¹H NMR (400 MHZ, DMSO-d6) |Ä 11.40 (s, 1H), 10.96 (d, J=3.5 Hz, 1H), 9.20 (d, J=5.1 Hz, 1H), 8.60 (dd, J=6.2, 2.2 Hz, 1H), 8.17 (d, J=2.5 Hz, 1H), 7.99 (ddd, J=8.1, 3.2, 2.2 Hz, 1H), 7.72 (dd, J=8.1, 4.4 Hz, 1H), 7.66 (d, J=2.2 Hz, 1H), 7.63 \ddot{ }C 7.54 (m, 1H), 7.50 (d, J=8.7 Hz, 1H), 6.20 (d, J=2.2 Hz, 1H), 4.63 (s, 1H), 4.53 (s, 1H), 3.83 (s, 3H), 3.78 (s, 3H), 2.97 (d, J=10.8 Hz, 3H), 2.85 (d, J=4.7 Hz, 3H), 2.15° C. 2.05 (m, 1H), 0.90 \ddot{ }C 0.82 (m, 4H).

### Example 224

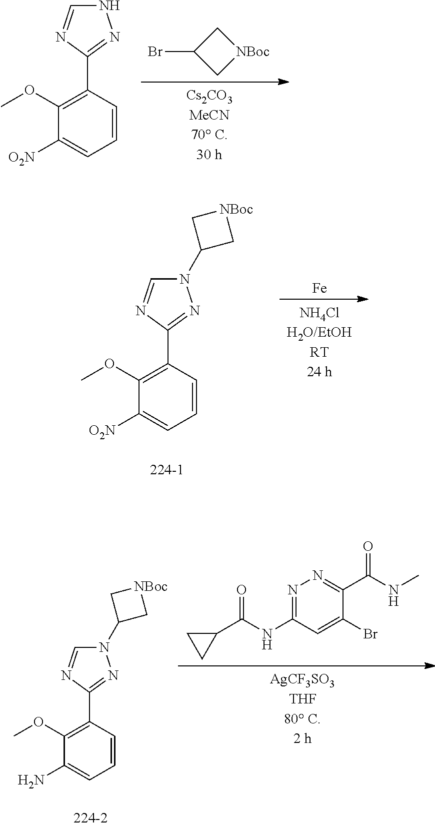

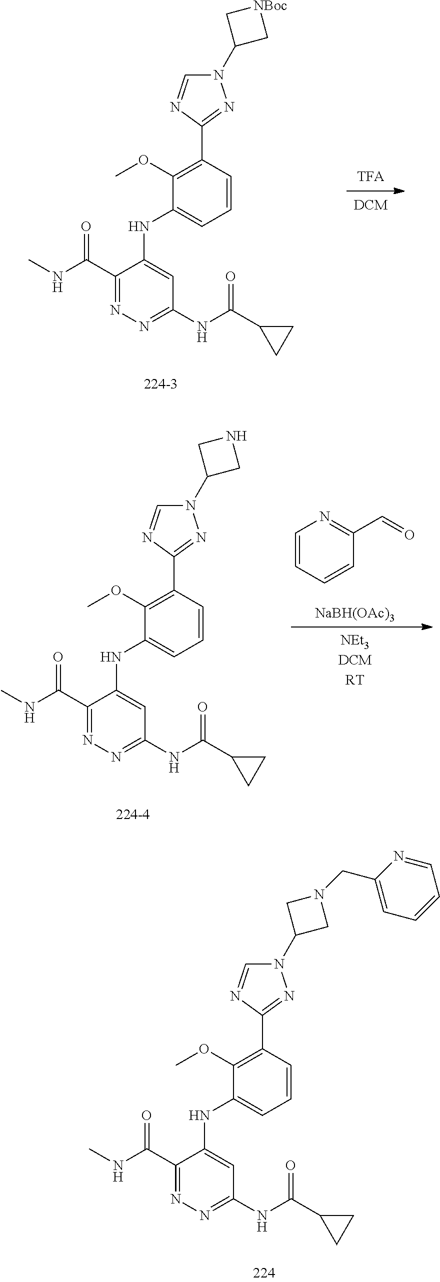 [SMILES: CNC(=O)c1nnc(NC(=O)C2CC2)cc1Nc1cccc(-c2ncn(C3CN(C(=O)OC(C)(C)C)C3)n2)c1OC ; CNC(=O)c1nnc(NC(=O)C2CC2)cc1Nc1cccc(-c2ncn(C3CNC3)n2)c1OC ; O=Cc1ccccn1 ; CNC(=O)c1nnc(NC(=O)C2CC2)cc1Nc1cccc(-c2ncn(C3CN(Cc4ccccn4)C3)n2)c1OC ; "NaBH(OAc)3"]

Step 1: To a stirred solution of 3-(2-methoxy-3-nitrophenyl)-1H-1,2,4-triazole (500 mg, 1.0 equiv, 2.27 mmol) and tert-butyl 3-bromoazetidine-1-carboxylate (643 mg, 1.2 equiv, 2.72 mmol) in MeCN (1 mL) were added Cs₂CO₃ (1.48 g, 2.0 equiv, 4.54 mmol) in portions at room temperature under nitrogen atmosphere. The resulting mixture was stirred for 24 hour at 70° C. The resulting mixture was extracted with EtOAc (2×20 mL). The combined organic layers were washed with water (2×20 mL), dried over anhydrous Na₂SO₄. After filtration, the filtrate was concentrated under reduced pressure. The residue was purified by silica gel column chromatography, eluted with petroleum ether/EtOAc (1:1) to afford compound 224-1 (500 mg, 1.33 mmol, 59% yield) as a white solid.

m/z (ES+) [M+H]+=376.1

Step 2: To a stirred solution of compound 224-1 (500 mg, 1.0 equiv, 1.33 mmol), H₂O (1 mL) and Fe (149 mg, 18.9 μL, 2.0 equiv, 2.66 mmol) in EtOH (1 mL) were added ammonium chloride (142 mg, 98.4 μL, 2.0 equiv, 2.66 mmol) in portions at room temperature under nitrogen atmosphere. The resulting mixture was stirred for 10 hour at 25° C. The reaction was quenched with sat. NH₄Cl (aq.) at room temperature. The aqueous layer was extracted with EtOAc (3×4 mL). The resulting mixture was concentrated under reduced pressure. The residue was dissolved in MeCN (5 mL). The crude product was purified by reverse phase flash with the following conditions (gradient: MeCN/water) to afford compound 224-2 (340 mg, 984 μmol, 74% yield) as a yellow solid.

m/z (ES+) [M+H]+=346.2

Step 3: To a stirred solution of 4-bromo-6-(cyclopropanecarboxamido)-N-methylpyridazine-3-carboxamide (500 mg, 1.0 equiv 1.67 mmol) and silver (I) trifluoromethanesulfonate (859 mg, 2.0 equiv, 3.34 mmol) in THF (10 mL) was added compound 224-2 (1.15 g, 2.0 equiv, 3.34 mmol) in portions at room temperature under nitrogen atmosphere. The resulting mixture was stirred for 3 hour at 70° C. The reaction was quenched with sat. NH₄Cl (aq.) at room temperature. The aqueous layer was extracted with EtOAc (3×4 mL). The resulting mixture was concentrated under reduced pressure. The residue was dissolved in MeCN (5 mL). The crude product was purified by reverse phase flash with the following conditions (gradient: MeCN/water) to afford compound 224-3 (500 mg, 887 μmol, 53% yield) as a yellow solid.

m/z (ES+) [M+H]+=564.2

Step 4: To a stirred solution of compound 224-3 (282 mg, 1.0 equiv, 500 μmol) in DCM (5 mL) were added TFA (57.0 mg, 38.5 μL, 1.0 equiv, 500 μmol) in portions at room temperature under nitrogen atmosphere. The resulting mixture was stirred for 5 hour at 25° C. The reaction was then quenched with sat. NH₄Cl (aq.) at room temperature. The aqueous layer was extracted with EtOAc (3×4 mL). The resulting mixture was concentrated under reduced pressure. The residue was dissolved in MeCN (5 mL). The crude product was purified by reverse phase flash chromatography with the following conditions (gradient: MeCN/water) to afford compound 224-4 (230 mg, 496 μmol, 99% yield) as a yellow solid.

m/z (ES+) [M+H]+=464.3

Step 5: A mixture of compound 224-4 (150 mg, 1.0 equiv, 324 μmol), picolinaldehyde (52.0 mg, 1.5 equiv, 485 μmol), triethylamine (65.5 mg, 90.2 μL, 2.0 equiv, 647 μmol) were stirred in DCM (5 mL) for 20 minutes at RT before the slow addition of sodium triacetoxyborohydride (137 mg, 2.0 equiv, 647 μmol) over 20 minutes. he resulting mixture was extracted with EtOAc. The combined organic layers were washed with water and then washed with brine, dried over anhydrous Na₂SO₄. After filtration, the filtrate was concentrated under reduced pressure. The crude product was purified by Prep-HPLC with the following conditions (Column: Xselect CSH C18 OBD column, gradient MeCN/0.1% formic acid in water) to afford compound 224 (16 mg, 29 μmol, 9% yield) as a light yellow amorphous solid.

m/z (ES+) [M+H]+=555.5

¹H NMR (400 MHZ, DMSO-d6) 11.34 (s, 1H), 11.03 (s, 1H), 9.18 (q, J=4.8 Hz, 1H), 8.76 (s, 1H), 8.50 (dt, J=4.8, 1.4 Hz, 1H), 8.18 (s, 1H), 7.88-7.64 (m, 2H), 7.54 (dd, J=8.0, 1.6 Hz, 1H), 7.41 (d, J=7.8 Hz, 1H), 7.34-7.21 (m, 2H), 5.24 (p, J=6.8 Hz, 1H), 4.02-3.56 (m, 9H), 2.87 (d, J=4.8 Hz, 3H), 2.09 (p, J=6.2 Hz, 1H), 0.99-0.68 (m, 4H).

### Example 227

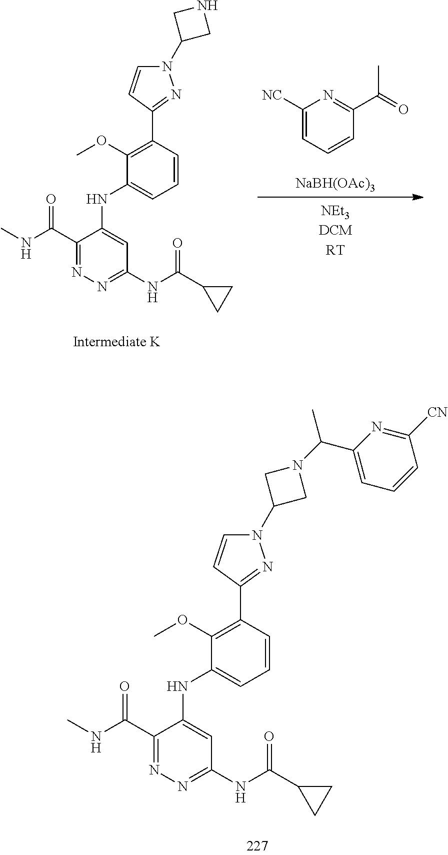 [SMILES: CNC(=O)c1nnc(NC(=O)C2CC2)cc1Nc1cccc(-c2ccn(C3CNC3)n2)c1OC ; CC(=O)c1cccc(C#N)n1 ; CNC(=O)c1nnc(NC(=O)C2CC2)cc1Nc1cccc(-c2ccn(C3CN(C(C)c4cccc(C#N)n4)C3)n2)c1OC ; "NaBH(OAc)3"]

A mixture of Intermediate K (77.0 mg, 1.20 equiv, 527 μmol), and triethylamine (133 mg, 184 μL, 3.0 equiv, 1.32 mmol) in DCM (6 mL) was stirred 50 minutes at RT followed by the slow addition of sodium triacetoxyborohydride (186 mg, 2.0 equiv, 878 μmol) over 60 minutes. The reaction was stirred over three days. The reaction was diluted with DCM (3 mL) and treated with 2N aqueous NaOH (3 mL). The layers were separated, the aqueous layer was extracted again with fresh DCM (5 mL), and the combined organic layers were dried over sodium sulfate, filtered, concentrated down, and dried. The crude was purified by silica gel chromatography (gradient: DCM/10:90:0.5 MeOH/DCM/NH₄OH) to provide compound rac-227 as a mixture of enantiomers (143.1 mg, 0.24 mmol, 54%, 99% Purity) as an off-white solid.

MS (ESI+): m/z=593.5 [M+H]⁺

¹H NMR (400 MHZ, DMSO) § 11.29 (s, 1H), 10.96 (s, 1H), 9.15 (q, J=4.7 Hz, 1H), 8.37 (d, J=0.8 Hz, 1H), 8.13 (s, 1H), 8.04 (t, J=7.8 Hz, 1H), 8.00 (d, J=0.7 Hz, 1H), 7.92 (dd, J=7.6, 1.1 Hz, 1H), 7.79 (dd, J=8.0, 1.1 Hz, 1H), 7.46 (dd, J=7.8, 1.6 Hz, 1H), 7.29 (dd, J=7.9, 1.6 Hz, 1H), 7.19 (t, J=7.9 Hz, 1H), 5.06 (p, J=6.9 Hz, 1H), 3.81 (t, J=7.1 Hz, 1H), 3.71 (q, J=6.5 Hz, 1H), 3.60-3.54 (m, 5H), 3.43 (t, J=7.0 Hz, 1H), 2.85 (d, J=4.8 Hz, 3H), 2.10-2.03 (m, 1H), 1.22 (d, J=6.6 Hz, 3H), 0.84-0.77 (m, 4H).

The enantiomers of compound 227 were separated by chiral SFC to provide compounds 283 and 286 (absolute stereochemistry arbitrarily assigned).

### Compound 283

MS (ESI+): m/z=593.3 [M+H]⁺

¹H NMR (400 MHZ, DMSO-d6) 11.32 (s, 1H), 10.98 (s, 1H), 9.18 (d, J=5.1 Hz, 1H), 8.39 (s, 1H), 8.15 (s, 1H), 8.11-8.00 (m, 2H), 7.95 (d, J=7.5 Hz, 1H), 7.81 (d, J=8.1 Hz, 1H), 7.48 (dd, J=7.8, 1.6 Hz, 1H), 7.30 (dd, J=7.9, 1.5 Hz, 1H), 7.21 (t, J=7.8 Hz, 1H), 5.08 (t, J=7.2 Hz, 1H), 3.83 (s, 1H), 3.72 (d, J=6.5 Hz, 1H), 3.59 (s, 5H), 3.45 (s, 1H), 2.87 (d, J=4.8 Hz, 3H), 2.08 (q, J=6.5, 6.1 Hz, 1H), 1.24 (d, J=6.4 Hz, 3H), 0.82 (dd, J=6.4, 3.6 Hz, 4H).

### Compound 286

MS (ESI+): m/z=593.3 [M+H]⁺

¹H NMR (400 MHZ, DMSO-d6) 11.32 (s, 1H), 10.98 (s, 1H), 9.18 (d, J=4.9 Hz, 1H), 8.39 (s, 1H), 8.15 (s, 1H), 8.05 (dd, J=15.5, 7.7 Hz, 2H), 7.94 (dd, J=7.7, 1.1 Hz, 1H), 7.81 (dd, J=8.1, 1.2 Hz, 1H), 7.48 (dd, J=7.8, 1.6 Hz, 1H), 7.30 (dd, J=7.9, 1.5 Hz, 1H), 7.21 (t, J=7.9 Hz, 1H), 5.09 (q, J=7.1 Hz, 1H), 3.83 (t, J=7.1 Hz, 1H), 3.72 (q, J=6.6 Hz, 1H), 3.59 (s, 5H), 3.44 (t, J=6.9 Hz, 1H), 2.87 (d, J=4.8 Hz, 3H), 2.09 (q, J=6.2, 5.8 Hz, 1H), 1.24 (d, J=6.6 Hz, 3H), 0.82 (dd, J=6.3, 3.4 Hz, 4H).

### Example 229

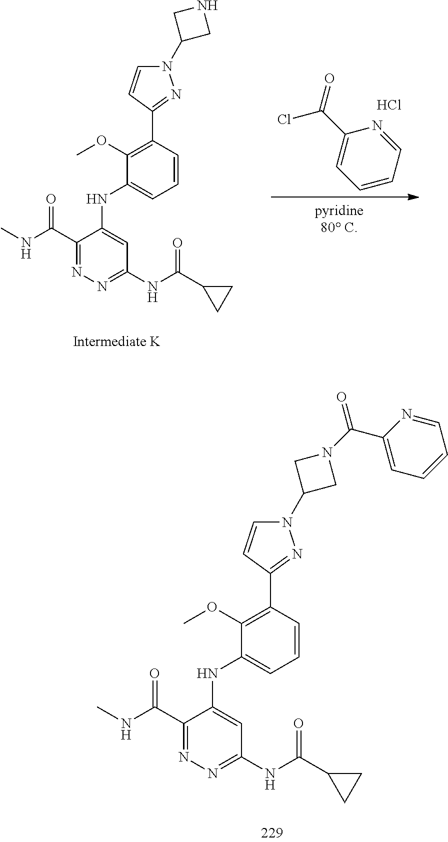 [SMILES: CNC(=O)c1nnc(NC(=O)C2CC2)cc1Nc1cccc(-c2ccn(C3CNC3)n2)c1OC ; O=C(Cl)c1ccccn1 ; Cl ; CNC(=O)c1nnc(NC(=O)C2CC2)cc1Nc1cccc(-c2ccn(C3CN(C(=O)c4ccccn4)C3)n2)c1OC]

A solution of Intermediate K (30 mg, 1.0 equiv, 65 μmol) and picolinoyl chloride hydrochloride (12 mg, 1.0 equiv, 65 μmol) in pyridine (0.5 mL) was stirred 90 hours at RT in a sealed tube. Only a trace amount of desired product was observed, so several additional equivalents of picolinoyl chloride hydrochloride were added, and the reaction was stirred at 85° C. for 5 days until the reaction was 50% complete by LC/MS analysis. A few drops of water were added to quench unreacted acid chloride. The reaction was then concentrated down, dried, and purified by silica gel chromatography (gradient: DCM/10:90:0.5 MeOH/DCM/NH₄OH) to yield compound 229 (5.5 mg, 8.9 μmol, 14% yield) as an off-white solid.

LC/MS (ES+): m/z=568.4 [M+H]+

¹H NMR (500 MHZ, DMSO) δ 11.28 (s, 1H), 10.95 (s, 1H), 9.14 (q, J=4.8 Hz, 1H), 8.64-8.60 (m, 1H), 8.39 (s, 1H), 8.13 (s, 1H), 8.07 (s, 1H), 8.01-7.95 (m, 2H), 7.55 (ddd, J=6.8, 4.7, 1.7 Hz, 1H), 7.47 (dd, J=7.9, 1.5 Hz, 1H), 7.29 (dd, J=7.9, 1.5 Hz, 1H), 7.19 (t, J=7.9 Hz, 1H), 5.41 (tt, J=8.0, 5.1 Hz, 1H), 5.08 (dd, J=10.8, 7.8 Hz, 1H), 4.90 (dd, J=10.8, 5.3 Hz, 1H), 4.58 (dd, J=10.6, 8.0 Hz, 1H), 4.40 (dd, J=10.6, 5.2 Hz, 1H), 3.59 (s, 3H), 2.85 (d, J=4.7 Hz, 3H), 2.06 (qd, J=7.2, 5.1 Hz, 1H), 0.84-0.78 (m, 4H).

### Example 231

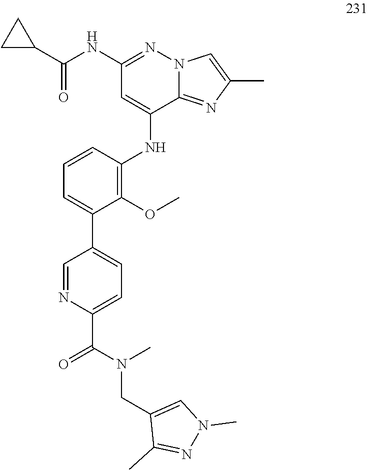

Compound 231 was prepared in analogous fashion to compound 219 in Example 219 except that 1-(1,3-dimethyl-1H-pyrazol-4-yl)-N-methylmethanamine was used in place of Intermediate N.

m/z (ES+) [M+H]+=580.3

¹H NMR (400 MHZ, DMSO-d6) 10.74 (s, 1H), 8.95 (d, J=9.0 Hz, 1H), 8.79 (dd, J=21.2, 2.2 Hz, 1H), 8.13 (ddd, J=8.2, 5.9, 2.2 Hz, 1H), 7.74 (d, J=1.0 Hz, 1H), 7.68 (dd, J=11.5, 8.0 Hz, 1H), 7.60 (d, J=11.2 Hz, 1H), 7.47 (dd, J=7.6, 1.9 Hz, 1H), 7.42-7.30 (m, 2H), 7.25 (d, J=3.0 Hz, 1H), 4.47 (d, J=26.9 Hz, 2H), 3.73 (d, J=14.7 Hz, 3H), 3.37 (d, J=1.5 Hz, 3H), 2.90 (d, J=18.2 Hz, 3H), 2.38 (s, 3H), 2.17 (s, 2H), 1.99-1.87 (m, 2H), 0.78 (d, J=6.2 Hz, 4H).

### Example 232

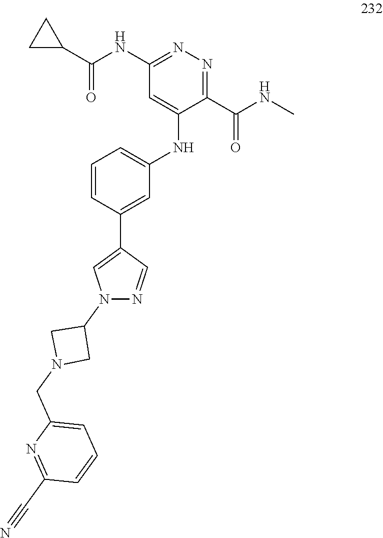 [SMILES: CNC(=O)c1nnc(NC(=O)C2CC2)cc1Nc1cccc(-c2cnn(C3CN(Cc4cccc(C#N)n4)C3)c2)c1]

Example 232 was prepared in analogous fashion to the compounds in Example 147 except that 3-bromoaniline was used in place of 3-bromo-2-methoxyaniline.

LC/MS (ES+) m/z=549.3

¹H NMR (400 MHZ, DMSO-d6) 11.31 (s, 1H), 10.82 (s, 1H), 9.17 (d, J=4.9 Hz, 1H), 8.46 (s, 1H), 8.15 (d, J=8.6 Hz, 1H), 8.07 (dd, J=13.9, 6.0 Hz, 2H), 7.97 (d, J=7.6 Hz, 1H), 7.78 (d, J=7.9 Hz, 1H), 7.57 (d, J=2.2 Hz, 1H), 7.48-7.40 (m, 2H), 7.14 (dt, J=7.0, 2.0 Hz, 1H), 5.11 (s, 1H), 4.00 (s, 5H), 2.86 (d, J=4.8 Hz, 3H), 2.16-1.97 (m, 1H), 0.89-0.74 (m, 4H).

### Example 233

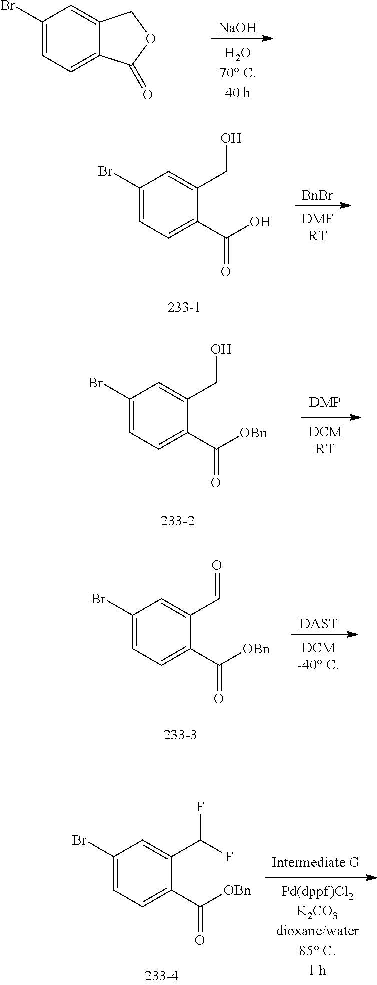

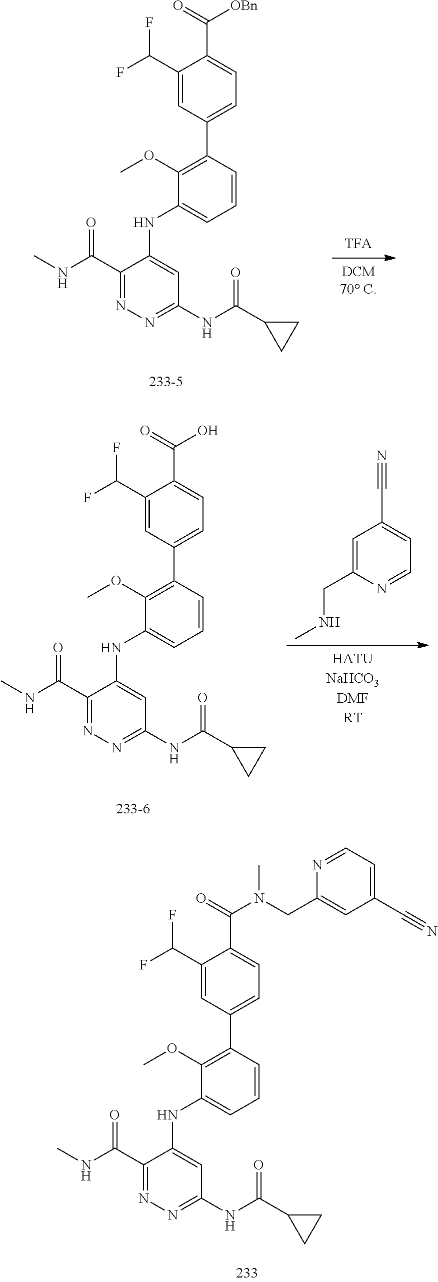 [SMILES: CNC(=O)c1nnc(NC(=O)C2CC2)cc1Nc1cccc(-c2ccc(C(=O)OCc3ccccc3)c(C(F)F)c2)c1OC ; CNC(=O)c1nnc(NC(=O)C2CC2)cc1Nc1cccc(-c2ccc(C(=O)O)c(C(F)F)c2)c1OC ; CNCc1cc(C#N)ccn1 ; CNC(=O)c1nnc(NC(=O)C2CC2)cc1Nc1cccc(-c2ccc(C(=O)N(C)Cc3cc(C#N)ccn3)c(C(F)F)c2)c1OC]

Step 1: To a stirred solution of 5-bromoisobenzofuran-1 (3H)-one (5 g, 1 equiv, 0.02 mol) in H₂O (50 mL) were added NaOH (2 g, 2 equiv, 0.05 mol) in portions at room temperature under nitrogen atmosphere. The resulting mixture was stirred for 5 hour at 70° C. after which the resulting mixture was concentrated under reduced pressure. The aqueous layer was extracted with EtOAc (3×50 mL). The resulting mixture was concentrated under reduced pressure. The residue was purified by silica gel column chromatography (petroleum ether/EtOAc 10:1 v/v) to afford compound 233-1 (4.5 g, 19 mmol, 80% yield) as a yellow solid.

m/z (ES+) [M+H]+=252.9

Step 2: To a stirred solution of compound 233-1 (5 g, 1 equiv, 0.02 mol) in DMF (50 mL) were added benzyl bromide (4 g, 1.1 equiv, 0.02 mol) in portions at room temperature under nitrogen atmosphere. The resulting mixture was stirred for 10 hour at 25° C. The reaction was then quenched with sat. NH₄Cl (aq.) at room temperature. The aqueous layer was extracted with EtOAc (3×4 mL). The resulting mixture was concentrated under reduced pressure. The residue was dissolved in MeCN (5 mL). The crude product was purified by reverse phase flash chromatography (gradient: MeCN/water) to afford compound 233-2 (4 g, 0.01 mol, 60% yield) as a yellow solid.

m/z (ES+) [M+H]+=320.9

Step 3: To a stirred solution of compound 233-2 (5 g, 1 Eq, 0.02 mol) in DCM (10 mL) was added Dess Martin periodinane (0.01 kg, 1.5 equiv, 0.02 mol) in portions at room temperature under nitrogen atmosphere. The resulting mixture was stirred for 2 hour at 25° C. The reaction was quenched with sat. NH₄Cl (aq.) at room temperature. The aqueous layer was extracted with EtOAc (3×4 mL). The resulting mixture was concentrated under reduced pressure. The residue was dissolved in MeCN (5 mL). The crude product was purified by reverse phase flash chromatography (gradient: MeCN/water) to provide compound 233-3 (3.5 g, 11 mmol, 70% yield) as a yellow solid.

m/z (ES+) [M+H]+=318.9

Step 4: Compound 233-3 (3.6 g, 1.0 equiv, 11 mmol) was stirred in THF (10 mL) for 20 minutes at RT followed by the slow addition of DAST (2.2 g, 1.8 mL, 1.2 equiv 14 mmol) over 20 minutes. The resulting mixture was extracted with EtOAc. The combined organic layers were washed with water and then washed with brine, dried over anhydrous Na₂SO₄. After filtration, the filtrate was concentrated under reduced pressure. The crude product was purified by Prep-HPLC(Column: Xselect CSH C18 OBD, gradient: MeCN/0.1% formic acid in water) to afford compound 233-4 (2.2 g, 6.4 mmol, 57% yield) as an off white amorphous solid that was used directly in the next step.

Step 5: A round bottomed flask was charged with compound 233-4 (400 mg, 1.0 equiv, 1.17 mmol), Intermediate G (903 mg, 2.0 equiv, 2.35 mmol), K₂CO₃ (324 mg, 2.0 equiv, 2.35 mmol), 1,4-Dioxane (10 mL), H₂O (3 mL) and a stirbar. PdCl₂ (dppf) (172 mg, 0.2 equiv, 235 μmol) was added, and the solution was stirred at 85° C. for 1 hour. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatograph to compound 233-5 (320 mg, 532 μmol, 45% yield).

m/z (ES+) [M+H]+=602.4

Step 6: To a stirred solution of compound 233-5 (400 mg, 1.0 equiv, 665 μmol) in DCM (5 mL) were added TFA (10 mL) in portions at room temperature under nitrogen atmosphere. The resulting mixture was stirred for 25° C. at 3 hour. The reaction was then quenched with sat. NH₄Cl (aq.) at room temperature. The aqueous layer was extracted with EtOAc (3×10 mL). The resulting mixture was concentrated under reduced pressure. The residue was dissolved in MeCN (5 mL). The crude product was purified by reverse phase flash with the following conditions (gradient: MeCN/water) to afford compound 233-6 (300 mg, 587 μmol, 88% yield) as a yellow solid.

m/z (ES+) [M+H]+=512.1

Step 7: A round bottomed flask was charged with compound 233-6 (200 mg, 1.0 equiv, 391 μmol) DIEA (101 mg, 136 μL, 2.0 equiv, 782 μmol), and HATU (297 mg, 2.0 equiv, 782 μmol). DMF (3 mL) was added, and the solution was stirred at 25° C. for 1 hour. The resulted solution was purified using C18 flash chromatography (gradient: MeCN/water). Lyophilization provided compound 233 (53.8 mg, 84.0 μmol, 22% yield) as a light yellow amorphous solid.

m/z (ES+) [M+H]+=641.4

¹H NMR (400 MHZ, DMSO-d6) 11.34 (d, J=5.1 Hz, 1H), 11.00 (d, J=13.4 Hz, 1H), 9.18 (s, 1H), 8.92-8.71 (m, 1H), 8.19 (d, J=12.5 Hz, 1H), 7.97-7.79 (m, 3H), 7.72-7.60 (m, 1H), 7.57-7.44 (m, 2H), 7.38-6.87 (m, 3H), 4.75 (d, J=118.1 Hz, 2H), 3.39 (s, 3H), 3.10-2.74 (m, 6H), 2.10 (s, 1H), 0.84 (d, J=6.5 Hz, 4H).

### Example 240

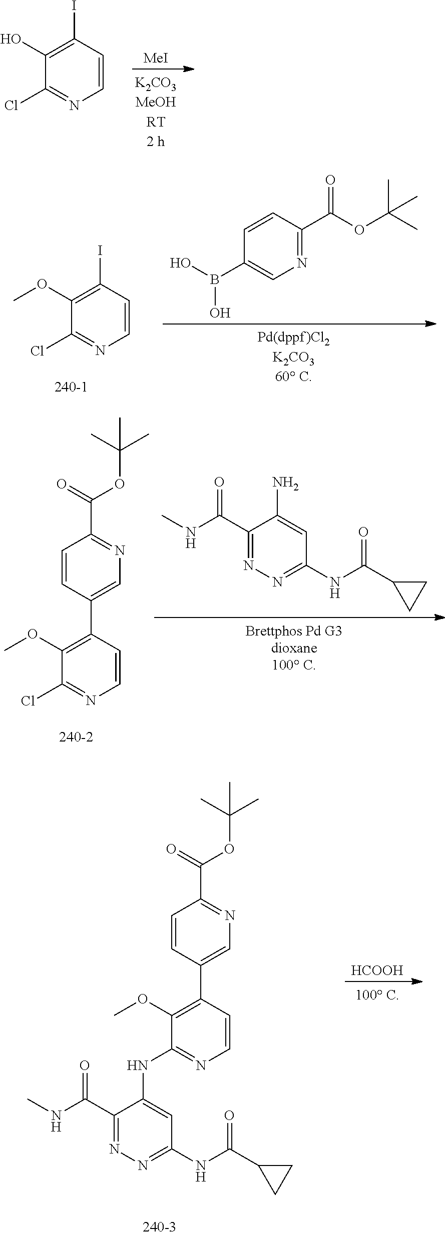

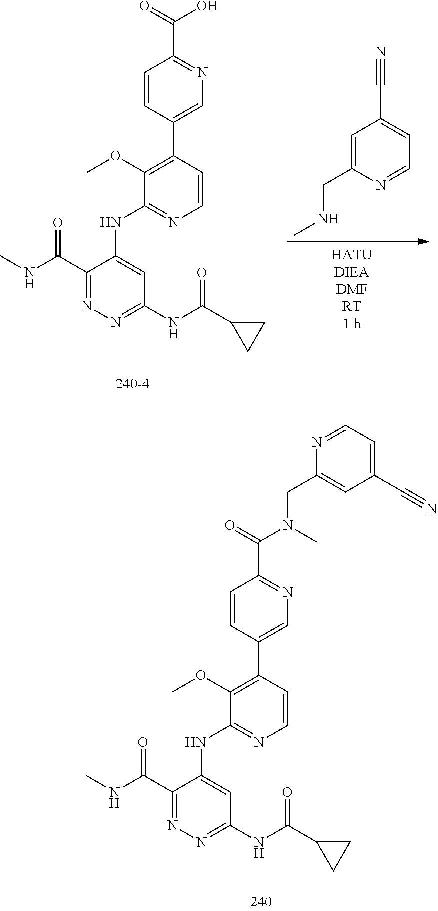 [SMILES: CNC(=O)c1nnc(NC(=O)C2CC2)cc1Nc1nccc(-c2ccc(C(=O)O)nc2)c1OC ; CNCc1cc(C#N)ccn1 ; CNC(=O)c1nnc(NC(=O)C2CC2)cc1Nc1nccc(-c2ccc(C(=O)N(C)Cc3cc(C#N)ccn3)nc2)c1OC]

Step 1: 2-Chloro-4-iodopyridin-3-ol (300 mg, 1.0 equiv, 1.17 mmol), iodomethane (200 mg, 1.2 equiv, 1.41 mmol) and K₂CO₃ (81.2 mg, 0.5 equiv, 587 μmol) were added to MeOH (10 mL). The mixture reacted at 24° C. for 3 hours. The mixture was extracted with water and and EtOAc. The organic layer was purified by reverse phase column chromatography (gradient: MeCN/water) to provide compound 240-1 (160 mg, 594 μmol, 51% yield) a white solid.

m/z (ES+) [M+H]+=269.90

Step 2: Compound 240-1 (150 mg, 1.0 equiv, 557 μmol) and (6-(tert-butoxycarbonyl)pyridin-3-yl) boronic acid (124 mg, 1.0 equiv, 557 μmol) were added to solvent DMF (1 mL) followed by the addition of PdCl₂ (dppf) (40.7 mg, 0.1 equiv, 55.7 μmol) and K₂CO₃ (76.9 mg, 1.0 equiv, 557 μmol) to the reaction mixture after which it was heated to 60° C. for 24 h. The mixture was extracted with water and and EtOAc. The organic layer was purified by reverse phase column chromatography (gradient: MeCN/water) to provide compound 240-2 (92 mg, 0.29 mmol, 52% yield) a white solid.

m/z (ES+) [M+H]+=351.1

Step 3: Compound 240-2 (100 mg, 1.0 equiv, 312 μmol) and 4-amino-6-(cyclopropanecarboxamido)-N-methylpyridazine-3-carboxamide (73.3 mg, 1.0 equiv, 312 μmol) were added to 1,4-dioxane (3 mL) followed by the addition of Brettphos Pd G3 (3 mg) to the reaction solution after which the mixture was heated at 100° C. for 1 h. The mixture reacted at 100° C. for 1 hour. The crude mixture was purified by reverse phase column chromatography (gradient: MeCN/water) to provide compound 240-3 (72 mg, 0.14 mmol, 44%) as a white solid.

m/z (ES+) [M+H]+=520.4

Step 4: Compound 240-3 (70 mg, 1.0 equiv, 0.13 mmol) was added to HCOOH (3 mL). The mixture reacted at 80° C. for 10 min. The crude mixture was purified by reverse phase column chromatography (gradient: MeCN/water) to provide compound 240-4 (24 mg, 52 μmol, 38% yield) as a white solid.

m/z (ES+) [M+H]+=464.3

Step 5: Compound 240-4 (24 mg, 1.0 equiv, 52 μmol) and 2-((methylamino)methyl) isonicotinonitrile (7.6 mg, 1.0 equiv, 52 μmol) were added to DMF (2 mL) after which DIEA (13 mg, 18 μL, 2.0 equiv, 0.10 mmol) and HATU (30 mg, 1.5 equiv, 78 μmol) were added to the reaction mixture. The mixture reacted at 24° C. for 1 hour. The reaction was then quenched with water and purified using PREP-HPLC (column: XBridge Prep OBD C18 Column, gradient: MeCN/10 mM NH₄HCO₃ in water) Lyophilization provided compound 240 (9.2 mg, 16 μmol, 30% yield) as a white amorphous solid.

m/z (ES+) [M+H]+=593.3

¹H NMR (400 MHZ, Chloroform-d) 12.62 (s, 1H), 10.11 (d, J=6.6 Hz, 1H), 9.41 (s, 1H), 8.97-8.91 (m, 1H), 8.83-8.73 (m, 1H), 8.34 (dd, J=12.4, 5.1 Hz, 1H), 8.21 (ddd, J=11.0, 8.1, 2.3 Hz, 2H), 7.95 (dd, J=41.4, 8.1 Hz, 1H), 7.79 (d, J=47.2 Hz, 1H), 7.49 (dd, J=5.0, 1.5 Hz, 1H), 7.01 (dd, J=19.9, 5.1 Hz, 1H), 4.99 (d, J=11.5 Hz, 2H), 3.63 (d, J=16.9 Hz, 3H), 3.31 (s, 2H), 3.18 (s, 1H), 3.09 (dd, J=5.1, 3.6 Hz, 3H), 1.79 (t, J=3.8 Hz, 1H), 1.24 (h, J=4.0 Hz, 2H), 1.02 (dq, J=6.5, 3.5 Hz, 2H).

### Example 245 and Example 246

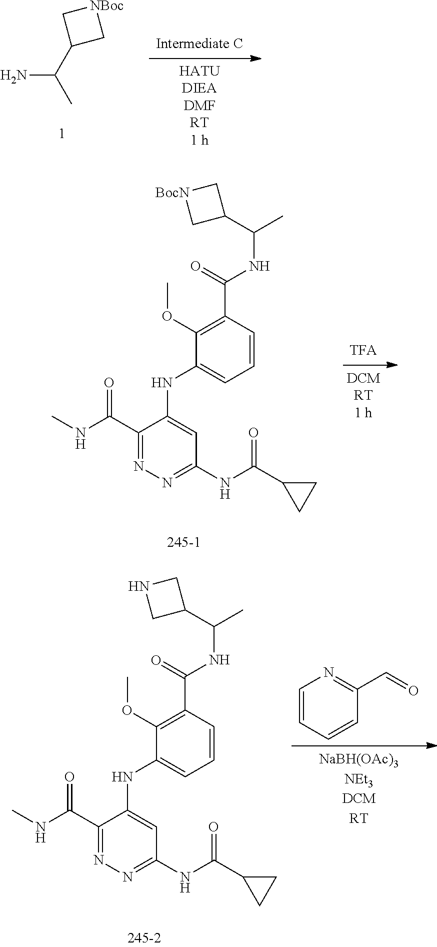 [SMILES: CC(N)C1CN(C(=O)OC(C)(C)C)C1 ; CNC(=O)c1nnc(NC(=O)C2CC2)cc1Nc1cccc(C(=O)NC(C)C2CN(C(=O)OC(C)(C)C)C2)c1OC ; CNC(=O)c1nnc(NC(=O)C2CC2)cc1Nc1cccc(C(=O)NC(C)C2CNC2)c1OC ; O=Cc1ccccn1]

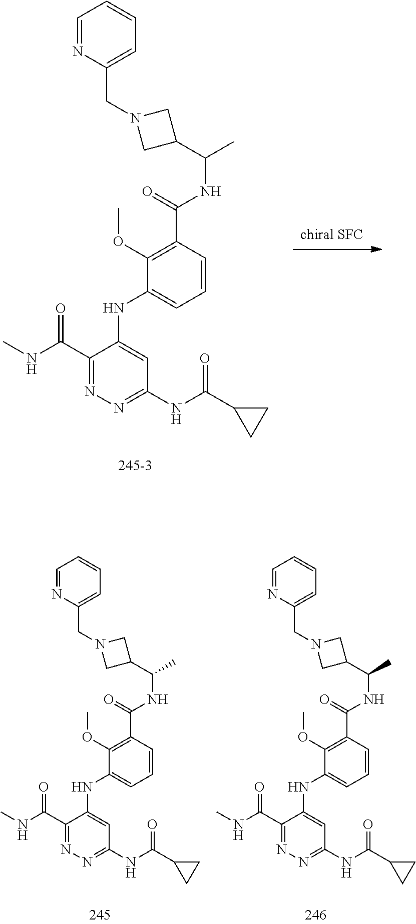 [SMILES: CNC(=O)c1nnc(NC(=O)C2CC2)cc1Nc1cccc(C(=O)NC(C)C2CN(Cc3ccccn3)C2)c1OC ; CNC(=O)c1nnc(NC(=O)C2CC2)cc1Nc1cccc(C(=O)N[C@@H](C)C2CN(Cc3ccccn3)C2)c1OC ; CNC(=O)c1nnc(NC(=O)C2CC2)cc1Nc1cccc(C(=O)N[C@H](C)C2CN(Cc3ccccn3)C2)c1OC]

Step 1: A resealable reaction vial was charged with tert-butyl 3-(1-aminoethyl) azetidine-1-carboxylate (300 mg, 1.2 equiv, 1.50 mmol), Intermediate C (481 mg, 1.0 equiv, 1.25 mmol), HATU (569 mg, 1.2 equiv, 1.50 mmol), DIEA (483 mg, 3 equiv, 3.74 mmol), and a stir bar before being evacuated and purged with nitrogen three times. DMF (12 mL) was added, and the mixture was stirred for 1 h at room temperature. The reaction mixture was diluted with H₂O (30 mL), and the aqueous phase was extracted with DCM (3×50 mL) three times. The combined organic layers were washed with brine, dried over sodium sulfate, filtered, and concentrated in vacuo. The resulting crude material was purified by C-18 flash column chromatography (gradient: MeCN/water) to provide compound 245-1 (500 mg, 71% yield) as a yellow solid.

m/z (ES+) [M+H]+=568.4

Step 2: A resealable reaction vial was charged compound 245-1 (330 mg, 1.0 equiv, 581 μmol), DCM/TFA (7 mL, 1:1 v/v), and a stir bar before being evacuated and purged with nitrogen three times. The mixture was stirred at room temperature for 1 h. The reaction liquid was dried under the vacuum pump to provide compound 245-2 (200 mg, 74% yield) as a yellow solid and used directly without further purification.

m/z (ES+) [M+H]+=468.2

Step 3: A resealable reaction vial was charged with compound 245-2 (200 mg, 1.0 equiv, 428 μmol), picolinaldehyde (55.0 mg, 1.2 equiv, 513 μmol), NaBH(OAc)₃ (181 mg, 2 equiv, 856 μmol), TEA (130 mg, 3.0 equiv, 1.28 mmol), and a stir bar before being evacuated and purged with nitrogen three times. DCM (5 mL) was added, and the mixture was stirred at room temperature. The reaction mixture was dropped into ice water, and the aqueous phase was extracted with DCM (3×50 mL) three times. The combined organic layers were washed with brine, dried over sodium sulfate, filtered, and concentrated in vacuo. The resulting crude material was purified by C-18 flash column chromatography (gradient: MeCN/water) (0˜100%) as eluent to provide racemic compound 245-3 (100 mg, 42% yield) as an off-white solid.

m/z (ES+) [M+H]+=559.3

Step 4: Compound 245 (100 mg, 1 Eq, 179 μmol) was separated by chiral-HPLC (Column: DZ-CHIRALPAK AD-3, 4.6*100 mm, 3.0 um; Mobile Phase A: Hex (0.2% DEA): (EtOH:IPA=2:1)=55:45; Flow rate: 1 mL/min; Gradient: 0% B to 0% B; Injection Volume: 5 ul mL). Lyophilization yielded compound 245-4 (28.1 mg, 28% yield) as a off-white amorphous solid, and compound 246 (24.8 mg, 25% yield) as a off-white amorphous solid (absolute stereochemistry arbitrarily assigned).

### Compound 245:

m/z (ES+) [M+H]+=559.4

¹H NMR (400 MHZ, DMSO-d6) 11.35 (s, 1H), 10.95 (s, 1H), 9.18 (d, J=5.0 Hz, 1H), 8.52-8.45 (m, 1H), 8.23 (d, J=8.4 Hz, 1H), 8.14 (s, 1H), 7.76 (td, J=7.7, 1.9 Hz, 1H), 7.53 (dd, J=7.7, 1.9 Hz, 1H), 7.36 (d, J=7.8 Hz, 1H), 7.32-7.18 (m, 3H), 4.23 (q, J=7.6 Hz, 1H), 3.78 (s, 2H), 3.70 (s, 3H), 3.43 (d, J=10.3 Hz, 2H), 3.16 (d, J=35.3 Hz, 2H), 2.86 (d, J=4.8 Hz, 3H), 2.57 (q, J=7.6 Hz, 1H), 2.13-2.01 (m, 1H), 1.08 (d, J=6.6 Hz, 3H), 0.88-0.76 (m, 4H).

### Compound 246:

m/z (ES+) [M+H]+=559.3

¹H NMR (400 MHZ, DMSO-d6) 11.35 (s, 1H), 10.95 (s, 1H), 9.18 (q, J=4.7 Hz, 1H), 8.57-8.36 (m, 1H), 8.23 (d, J=8.4 Hz, 1H), 8.14 (s, 1H), 7.76 (td, J=7.7, 1.9 Hz, 1H), 7.53 (dd, J=7.7, 1.9 Hz, 1H), 7.36 (d, J=7.8 Hz, 1H), 7.31-7.17 (m, 3H), 4.30-4.13 (m, 1H), 3.77 (s, 2H), 3.70 (s, 3H), 3.51-3.38 (m, 2H), 3.15 (d, J=34.6 Hz, 2H), 2.86 (d, J=4.8 Hz, 3H), 2.64-2.53 (m, 1H), 2.08 (qd, J=7.4, 5.1 Hz, 1H), 1.08 (d, J=6.6 Hz, 3H), 0.91-0.76 (m, 4H).

### Example 247

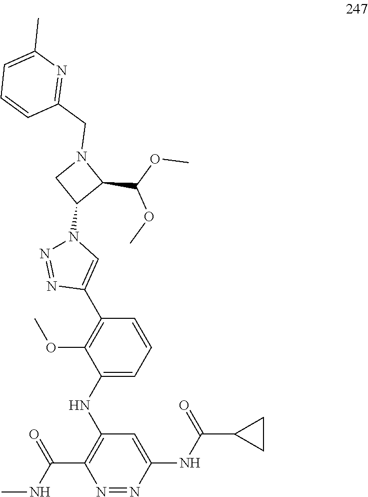 [SMILES: CNC(=O)c1nnc(NC(=O)C2CC2)cc1Nc1cccc(-c2cn([C@@H]3CN(Cc4cccc(C)n4)[C@H]3C(OC)OC)nn2)c1OC]

Compound 247 was prepared in analogous fashion to the procedures in Example 150 except that tert-butyl 3-azido-2-(dimethoxymethyl) azetidine-1-carboxylate was used in place of tert-butyl 3-azidoazetidine-1-carboxylate. It was isolated as a racemic mixture of the trans diastereomers.

m/z (ES+) [M+H]+=643.2

¹H NMR (500 MHZ, DMSO) δ 11.30 (s, 1H), 10.98 (s, 1H), 9.16 (d, J=4.9 Hz, 1H), 8.56 (s, 1H), 8.11 (s, 1H), 7.91 (dd, J=7.9, 1.6 Hz, 1H), 7.65 (t, J=7.6 Hz, 1H), 7.43 (dd, J=7.9, 1.6 Hz, 1H), 7.30 (t, J=7.9 Hz, 1H), 7.20 (d, J=7.6 Hz, 1H), 7.13 (d, J=7.6 Hz, 1H), 5.26 (q, J=7.5 Hz, 1H), 4.48 (d, J=6.2 Hz, 1H), 4.01 (d, J=13.9 Hz, 1H), 3.90 (t, J=6.7 Hz, 1H), 3.85-3.75 (m, 2H), 3.63 (s, 3H), 3.49 (t, J=7.5 Hz, 1H), 3.25 (d, J=13.7 Hz, 5H), 3.13 (s, 1H), 2.86 (d, J=4.8 Hz, 3H), 2.46 (s, 7H), 2.44 (s, 3H), 2.06 (q, J=6.9, 6.2 Hz, 1H), 0.84-0.78 (m, 4H).

### Example 256

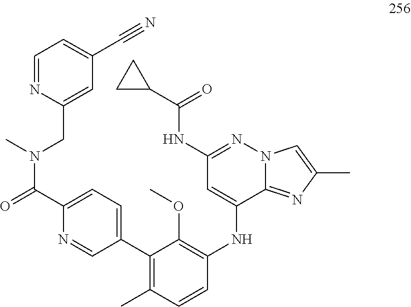

Compound 256 was prepared in analogous fashion using the procedures in example 219 except that 3-bromo-2-methoxy-4-methylaniline was used in place of 3-bromo-2-methoxyaniline.

m/z (ES+) [M+H]⁺=602.2

¹H NMR (400 MHZ, DMSO-d6) 10.75 (d, J=7.1 Hz, 1H), 8.97-8.74 (m, 2H), 8.50 (dd, J=63.4, 2.2 Hz, 1H), 8.02-7.64 (m, 5H), 7.41-7.15 (m, 3H), 4.90 (d, J=11.9 Hz, 2H), 3.30 (s, 2H), 3.23 (s, 1H), 3.11 (d, J=26.7 Hz, 3H), 2.38 (d, J=2.8 Hz, 3H), 2.11 (d, J=24.8 Hz, 3H), 1.92 (dt, J=12.3, 6.6 Hz, 1H), 0.88-0.71 (m, 4H).

### Example 262

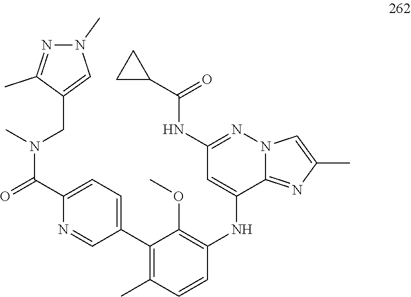

Compound 262 was prepared in analogous fashion to compound 256 except that 1-(1,3-dimethyl-1H-pyrazol-4-yl)-N-methylmethanamine was used in place of 2-((methylamino)methyl) isonicotinonitrile.

m/z (ES+) [M+H]+=594.3

¹H NMR (400 MHZ, DMSO-d6) 10.70 (s, 1H), 8.82 (d, J=6.6 Hz, 1H), 8.54 (ddd, J=20.4, 2.1, 0.9 Hz, 1H), 7.91 (td, J=7.7, 2.2 Hz, 1H), 7.73-7.63 (m, 2H), 7.59 (d, J=14.3 Hz, 1H), 7.35 (dd, J=8.1, 1.2 Hz, 1H), 7.23-7.15 (m, 2H), 4.48 (d, J=23.1 Hz, 2H), 3.72 (d, J=15.9 Hz, 3H), 3.28 (d, J=1.1 Hz, 3H), 2.91 (d, J=23.8 Hz, 3H), 2.37 (d, J=0.9 Hz, 3H), 2.18 (s, 2H), 2.12 (d, J=2.5 Hz, 3H), 1.92 (d, J=7.7 Hz, 2H), 0.78 (d, J=6.2 Hz, 4H).

### Example 268

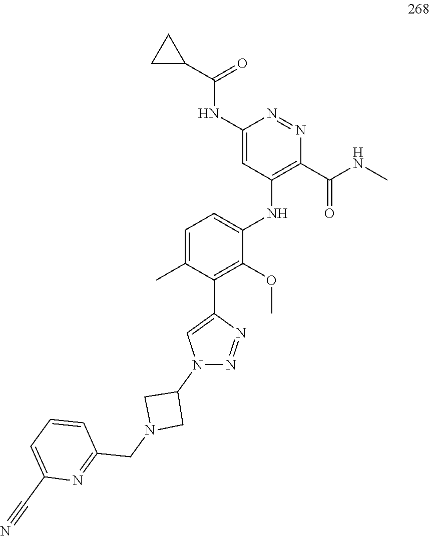 [SMILES: CNC(=O)c1nnc(NC(=O)C2CC2)cc1Nc1ccc(C)c(-c2cn(C3CN(Cc4cccc(C#N)n4)C3)nn2)c1OC]

Compound 268 was prepared in analogous fashion using the procedures from Example 150 except that Intermediate I was used instead of Intermediate H.

m/z (ES+) [M+H]+=594.1

¹H NMR (400 MHZ, DMSO) δ 11.28 (s, 1H), 10.80 (s, 1H), 9.12 (q, J=4.7 Hz, 1H), 8.51 (s, 1H), 8.10-7.99 (m, 2H), 7.91 (ddd, J=11.8, 7.6, 1.1 Hz, 1H), 7.77 (ddd, J=8.0, 6.4, 1.1 Hz, 1H), 7.38 (d, J=8.2 Hz, 1H), 7.19-7.12 (m, 1H), 5.41 (p, J=6.5 Hz, 1H), 4.59 (d, J=5.9 Hz, 1H), 3.96-3.88 (m, 4H), 3.72-3.64 (m, 2H), 3.42 (s, 3H), 2.83 (d, J=4.8 Hz, 3H), 2.23 (s, 3H), 2.06 (h, J=5.9, 5.5 Hz, 1H), 0.85-0.78 (m, 4H).

### Example 269

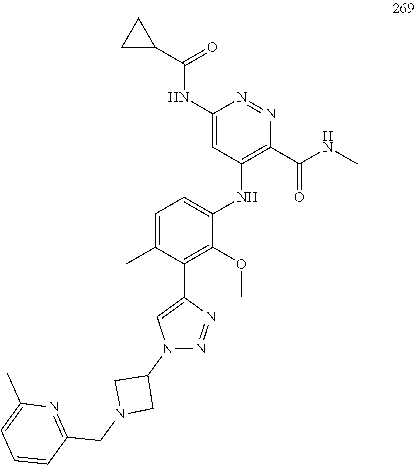 [SMILES: CNC(=O)c1nnc(NC(=O)C2CC2)cc1Nc1ccc(C)c(-c2cn(C3CN(Cc4cccc(C)n4)C3)nn2)c1OC]

Compound 269 was prepared in analogous fashion using the procedures from Example 150 except that Intermediate I was used instead of Intermediate H.

m/z (ES+) [M+H]+=583.0

¹H NMR (400 MHZ, DMSO) δ 11.28 (s, 1H), 10.79 (s, 1H), 9.12 (q, J=4.7 Hz, 1H), 8.48 (s, 1H), 8.08 (s, 1H), 7.63 (t, J=7.7 Hz, 1H), 7.37 (d, J=8.2 Hz, 1H), 7.23-7.13 (m, 2H), 7.11 (d, J=7.6 Hz, 1H), 5.44-5.33 (m, 1H), 3.89 (td, J=7.1, 1.5 Hz, 2H), 3.81 (s, 2H), 3.68-3.57 (m, 2H), 3.41 (s, 3H), 2.83 (d, J=4.8 Hz, 3H), 2.43 (s, 3H), 2.23 (s, 3H), 2.12-2.01 (m, 1H), 0.85-0.78 (m, 4H).

### Example 270

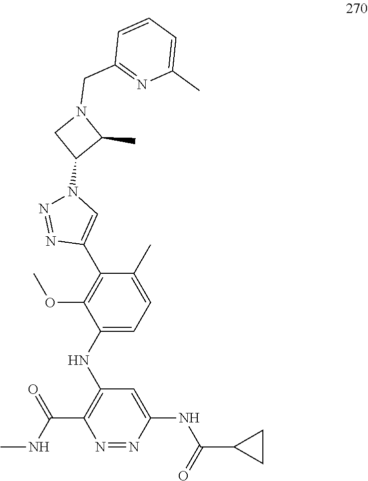 [SMILES: CNC(=O)c1nnc(NC(=O)C2CC2)cc1Nc1ccc(C)c(-c2cn([C@@H]3CN(Cc4cccc(C)n4)[C@H]3C)nn2)c1OC]

Compound 270 was prepared in analogous fashion using the procedures from Example 150 except that Intermediate I was used instead of Intermediate H and tert-butyl 3-azido-2-(dimethoxymethyl) azetidine-1-carboxylate was used in place of tert-butyl 3-azidoazetidine-1-carboxylate. It was isolated as a racemic mixture of the trans diastereomers.

m/z (ES+) [M+H]⁺=597.1

¹H NMR (400 MHZ, DMSO) δ 11.28 (s, 1H), 10.79 (s, OH), 9.12 (d, J=4.9 Hz, OH), 8.07 (s, OH), 7.68-7.60 (m, 1H), 7.60 (d, J=7.8 Hz, 1H), 7.35 (dd, J=20.8, 8.1 Hz, 1H), 7.23 (d, J=7.7 Hz, 1H), 7.10 (tt, J=17.8, 8.8 Hz, 3H), 3.95-3.83 (m, 1H), 3.70 (dd, J=15.6, 10.0 Hz, 1H), 3.53 (s, 2H), 3.47-3.39 (m, OH), 3.40 (s, 1H), 3.16 (s, 3H), 3.03 (s, 1H), 2.84 (d, J=4.8 Hz, 2H), 2.59 (s, 1H), 2.43 (d, J=6.7 Hz, 5H), 2.22 (s, 1H), 2.07 (p, J=6.3 Hz, 1H), 1.90 (s, 1H), 1.25-1.17 (m, 2H), 1.10 (d, J=5.4 Hz, 3H), 0.85-0.74 (m, 2H).

### Example 272

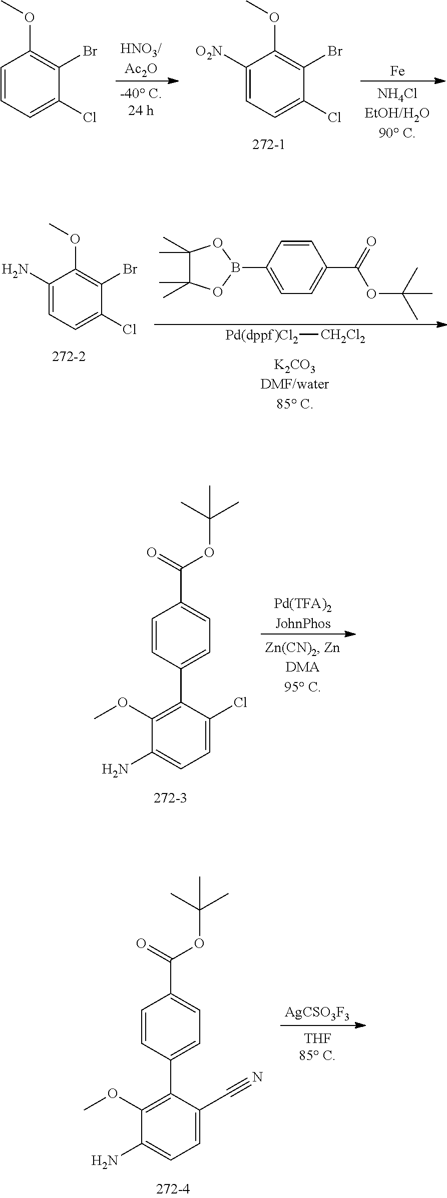

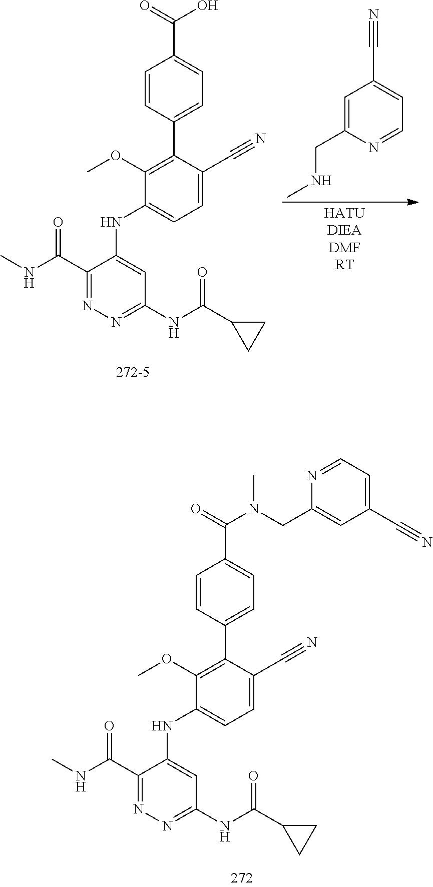 [SMILES: CNC(=O)c1nnc(NC(=O)C2CC2)cc1Nc1ccc(C#N)c(-c2ccc(C(=O)O)cc2)c1OC ; CNCc1cc(C#N)ccn1 ; CNC(=O)c1nnc(NC(=O)C2CC2)cc1Nc1ccc(C#N)c(-c2ccc(C(=O)N(C)Cc3cc(C#N)ccn3)cc2)c1OC]

Step 1: To a stirred solution of 2-bromo-1-chloro-3-methoxybenzene (5 g, 1 equiv, 0.02 mol) in Ac₂O (60 mL) was added HNO₃ (20 mL) at −40° C. The resulting mixture was stirred at room temperature for 12 h. The reaction was quenched with water and extracted with ethyl acetate. The organic layer was washed with brine, dried over Na₂SO₄ and evaporated to provide compound 272-1 (3 g, 0.01 mol, 50% yield) as a white solid.

m/z (ES+) [M+H]+=266.0

Step 2: To a stirred solution of compound 272-1 (2 g, 1 equiv, 8 mmol) and NH₄Cl (2 g, 4 equiv, 0.03 mol) in EtOH (21 mL) and H₂O (14 mL) was added Fe (2 g, 5 equiv, 0.04 mol) at room temperature. The resulting mixture was stirred at 90° C. for 2 h. The resulting mixture was filtered, the filter cake was washed with EtOH. The filtrate was quenched with water and extracted with ethyl acetate. The organic layer was washed with brine, dried over Na₂SO₄ and evaporated. This resulted in compound 272-2 (1.8 g, 7.6 mmol, 100%) as a white solid.

m/z (ES+) [M+H+2]+=238.0

Step 3: To a stirred solution of compound 272-2 (1.7 g, 1.0 equiv, 7.2 mmol), tert-butyl 4-(4,4,5,5-tetramethyl-1,3,2-dioxaborolan-2-yl)benzoate (2.2 g, 1.0 equiv, 7.2 mmol), K₂CO₃ (3.0 g, 3.0 equiv, 22 mmol) in DMF (20 mL) and H₂O (5.0 mL) was added Pd(dppf)Cl₂ (1.2 g, 0.2 equiv, 1.4 mmol) and the resulting solution was stirred at 85° C. for 1 hour under nitrogen atmosphere. The resulted solution was purified using C18 flash chromatography (gradient: MeCN/water) to provide compound 272-3 (1.6 g, 4.8 mmol, 67%) as a white solid.

m/z (ES+) [M+H]+=334.2

Step 4: To a stirred solution of compound 272-3 (1 g, 1 equiv, 3 mmol), 2-[di(tert-butyl)phosphino]-1,1′-binaphthyl (0.1 g, 0.088 equiv, 0.3 mmol), Zn(CN)₂ (0.2 g, 0.56 equiv, 2 mmol) and Zn (0.04 g, 0.19 equiv, 0.6 mmol) in DMA (10 mL) was added Pd(TFA)₂ (0.04 g, 0.043 Eq, 0.1 mmol) and the resulting solution was stirred at 95° C. for 14 hour under nitrogen atmosphere. The resulted solution was purified using C18 flash chromatography (gradient: MeCN/water) This resulted in compound 272-4 (500 mg, 1.54 mmol, 50% yield) as a white solid.

m/z (ES+) [M+H]+=325.2

Step 5: To a stirred solution of 4-bromo-6-(cyclopropanecarboxamido)-N-methylpyridazine-3-carboxamide (300 mg, 1.0 equiv, 1.00 mmol) and compound 272-4 (325 mg, 1 Eq, 1.00 mmol) in THF (1 mL) was added Silver (I) TrifluoromethaneSulfonate (515 mg, 2.0 equiv, 2.01 mmol) at room temperature. The resulting mixture was stirred at 85° C. for 1 h after which it was concentrated under reduced pressure and was dissolved in DMF. The resulted solution was purified using C18 flash chromatography (gradient: MeCN/water) to provide compound 272-5 (180 mg, 370 μmol, 36.9%) as a yellow solid.

m/z (ES+) [M+H]+=487.3

Step 6: To a stirred solution of compound 272-5 (53.2 mg, 1.1 equiv, 362 μmol) and NaHCO₃ (0.36 g, 13 equiv, 4.28 mmol) in DMF (2.0 mL) was added HATU (188 mg, 1.5 equiv, 493 μmol) at room temperature. The resulting mixture was stirred at room temperature for 1 h. The resulted solution was purified using prep-HPLC with following conditions (column: XBridge Prep OBD C18 Column, gradient: MeCN, 10 mM NH₄HCO₃ in water). This resulted in compound 272 (51.1 mg, 83.0 μmol, 25% yield) as a white amorphous solid.

m/z (ES+) [M+H]+=616.3.

¹H NMR (Chloroform-d, 400 MHz) 11.73 (1H, s), 9.10 (1H, s), 8.80 (1H, d, J=5.0 Hz), 8.57 (1H, s), 8.16 (1H, d, J=5.1 Hz), 7.77-7.34 (8H, m), 4.84 (2H, d, J=87.1 Hz), 3.43 (3H, s), 3.17 (3H, s), 3.07 (3H, d, J=5.0 Hz), 1.76 (1H, s), 1.22-1.16 (2H, m), 1.02 (2H, dq, J=7.7, 4.2 Hz).

### Example 278

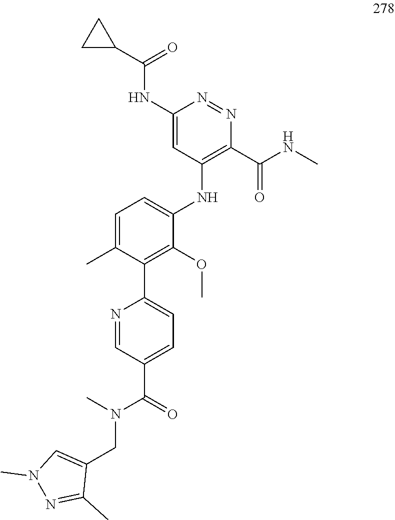

Compound 278 was prepared using analogous procedures to those in Example 55 except that 3-bromo-2-methoxy-4-methylaniline was used in place of 3-bromo-2-methoxyaniline.

m/z (ES+) [M+H]+=597.7

¹H NMR (400 MHz, DMSO-d6) 11.31 (s, 1H), 10.79 (s, 1H), 9.13 (q, J=4.8 Hz, 1H), 8.73 (d, J=2.2 Hz, 1H), 8.12 (s, 1H), 7.95 (dd, J=8.0, 2.3 Hz, 1H), 7.64 (s, 1H), 7.50 (d, J=8.0 Hz, 1H), 7.41 (d, J=8.2 Hz, 1H), 7.16 (d, J=8.2 Hz, 1H), 4.50 (s, 1H), 4.32 (s, 1H), 3.73 (s, 3H), 3.35 (s, 3H), 2.95 (s, 1H), 2.90° C. 2.81 (m, 5H), 2.17 (s, 2H), 2.08 (dd, J=8.7, 3.7 Hz, 1H), 2.05 (s, 3H), 1.88 (s, 1H), 0.87 \ddot{ }C 0.81 (m, 4H)

### Example 280

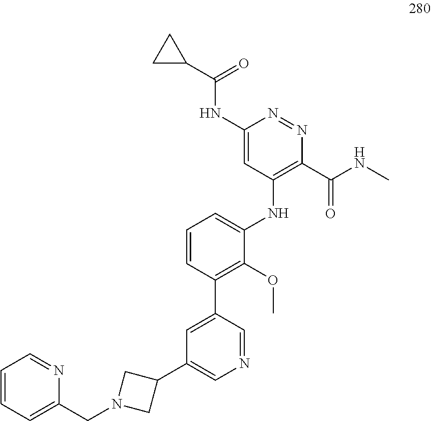 [SMILES: CNC(=O)c1nnc(NC(=O)C2CC2)cc1Nc1cccc(-c2cncc(C3CN(Cc4ccccn4)C3)c2)c1OC]

Compound 280 was prepared using analogous procedures as those in Example 182 except that tert-butyl 3-(5-bromopyridin-3-yl) azetidine-1-carboxylate was used in place of 2-(azetidin-3-yl)-5-bromopyridine.

MS (ES+) m/z=565.1 [M+H]+

### Example 301

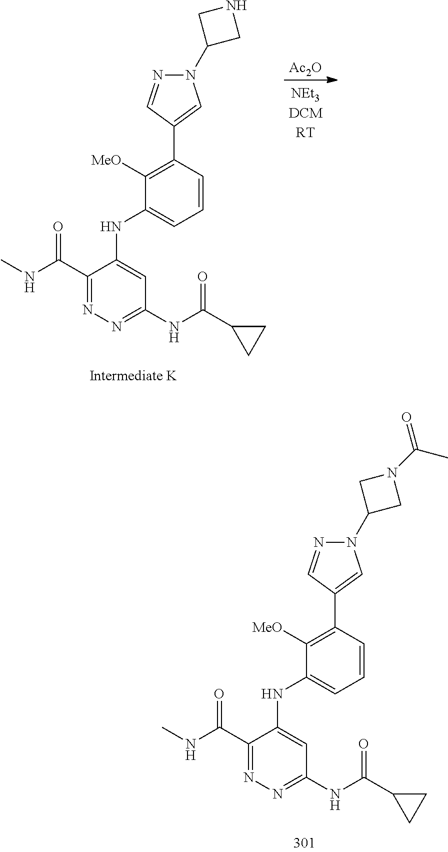 [SMILES: "Ac2O" ; CNC(=O)c1nnc(NC(=O)C2CC2)cc1Nc1cccc(-c2cnn(C3CNC3)c2)c1OC ; CNC(=O)c1nnc(NC(=O)C2CC2)cc1Nc1cccc(-c2cnn(C3CN(C(C)=O)C3)c2)c1OC]

In a 2-dram vial was charged Intermediate K (75 mg, 1.0 equiv, 0.16 mmol) in 1.5 mL of DCM and pyridine (38 mg, 39 μL, 3.0 equiv, 0.49 mmol), after which acetyl chloride (15 mg, 14 μL, 1.2 equiv, 0.19 mmol) was added in 1 mL of DCM dropwise, and then stirred for 1 hr. The reaction was then partitioned between DCM and water. The organics were dry loaded on silica gel and purified by flash column chromatography to provide compound 301.

MS (ES+) m/z=505.5 [M+H]+

¹H NMR (500 MHz, DMSO) δ 11.29 (s, 1H), 10.95 (s, 1H), 9.14 (q, J=4.8 Hz, 1H), 8.36 (s, 1H), 8.13 (s, 1H), 8.07 (s, 1H), 7.47 (dd, J=7.8, 1.5 Hz, 1H), 7.29 (dd, J=7.8, 1.6 Hz, 1H), 7.19 (t, J=7.9 Hz, 1H), 5.32 (tt, J=8.1, 5.3 Hz, 1H), 4.61-4.54 (m, 1H), 4.43 (dd, J=9.0, 5.3 Hz, 1H), 4.30 (dd, J=9.9, 8.1 Hz, 1H), 4.14 (dd, J=10.0, 5.4 Hz, 1H), 3.58 (s, 3H), 2.85 (d, J=4.9 Hz, 3H), 2.07 (tt, J=7.2, 5.2 Hz, 1H), 1.82 (s, 3H), 1.22 (s, 1H), 0.86-0.76 (m, 4H).

### Example 305

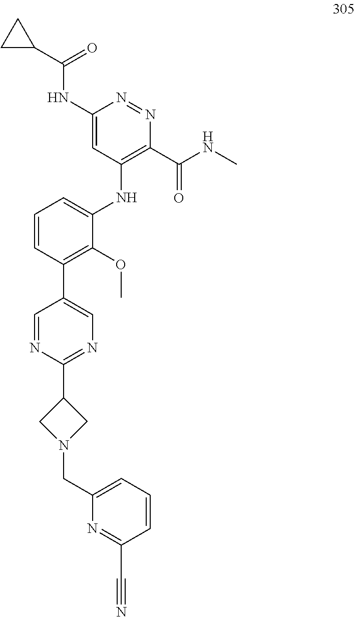 [SMILES: CNC(=O)c1nnc(NC(=O)C2CC2)cc1Nc1cccc(-c2cnc(C3CN(Cc4cccc(C#N)n4)C3)nc2)c1OC]

Compound 305 was prepared using analogous procedures as those in Example 182 except that tert-butyl 3-(5-bromopyrimidin-2-yl) azetidine-1-carboxylate was used in place of 2-(azetidin-3-yl)-5-bromopyridine and the Boc group was deprotected under standard TFA/DCM deprotection conditions.

LC/MS (ESI+): m/z=591.4 [M+H]+

TABLE 20
The following compounds were prepared using analogous procedures as
those used for the formation of Example 305.

| | | LCMS | |
|---|---|---|---|
| Com- | | m/z | |
| pound | Structure | (ES+) | ¹H NMR |
| 325 | 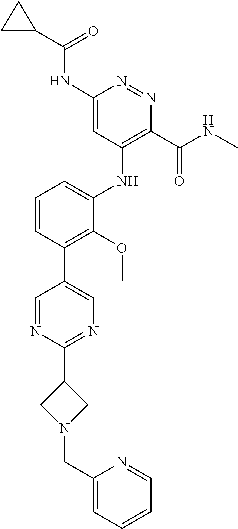 [SMILES: CNC(=O)c1nnc(NC(=O)C2CC2)cc1Nc1cccc(-c2cnc(C3CN(Cc4ccccn4)C3)nc2)c1OC] | 566.1 [M + H]+ | |
| 326 | 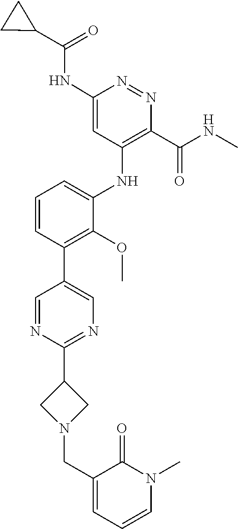 [SMILES: CNC(=O)c1nnc(NC(=O)C2CC2)cc1Nc1cccc(-c2cnc(C3CN(Cc4cccn(C)c4=O)C3)nc2)c1OC] | 596.2 [M + H]+ | |
| 327 | 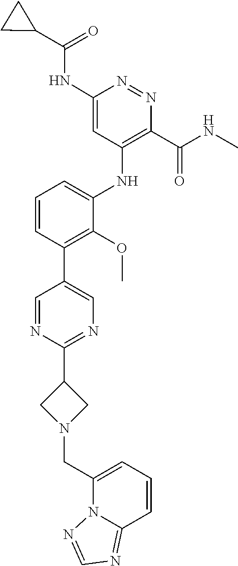 [SMILES: CNC(=O)c1nnc(NC(=O)C2CC2)cc1Nc1cccc(-c2cnc(C3CN(Cc4cccc5ncnn45)C3)nc2)c1OC] | 606.4 [M + H]+ | |

### Example 310

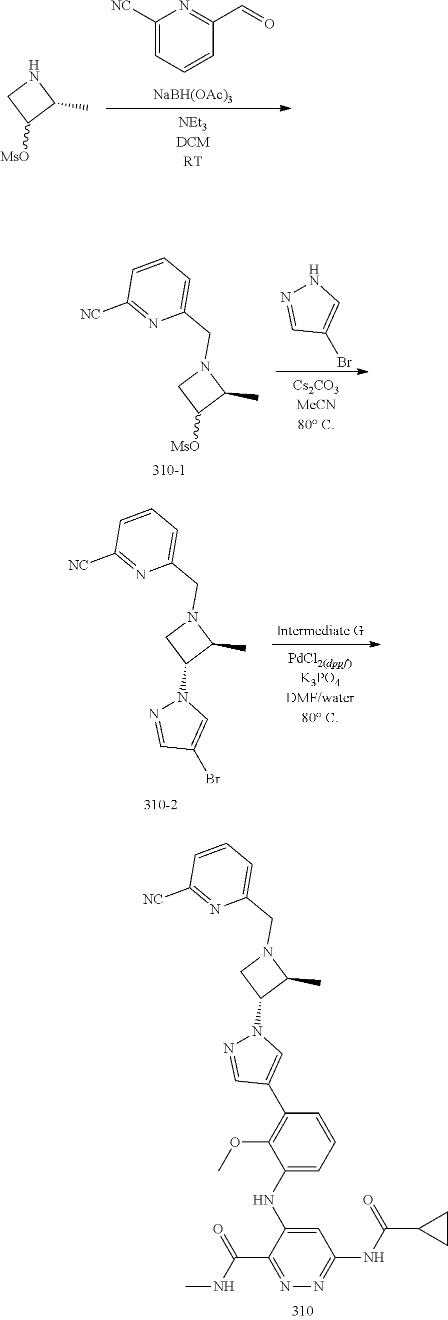

Step 1: In a 30 ml vial was charged (2S)-2-methylazetidin-3-yl methanesulfonate (300 mg, 1.0 equiv, 1.82 mmol), 6-formylpicolinonitrile (480 mg, 2.0 equiv, 3.63 mmol) in 3 mL of DCM after which TEA (735 mg, 1.01 mL, 4.0 equiv, 7.26 mmol) was added and stirred for 10 min. Sodium triacetoxyborohydride (770 mg, 2.0 equiv, 3.63 mmol) was then added followed by continued stirring for 2 hrs until the reaction was determined to be complete by LC/MS analysis. The reaction was then diluted with DCM and water. The organics were separated washed with brine, dried and concentrated to provide compound 310-1 that was used directly in the next step.

Step 2: Compound 310-1 was taken in with 4-bromo-1H-pyrazole (267 mg, 1.0 equiv, 1.82 mmol) in 3 mL of ACN and followed by the addition of cesium carbonate (1.18 g, 2.0 equiv, 3.63 mmol) and the reaction was heated to 80° C. for 45 min. Complete conversion to desired was observed by LC/MS analysis. Reaction was then cooled to RT and diluted with DCM and water. The organics were washed with water once more, then brine, dried and concentrated. The crude material was purified by flash silica gel chromatography (gradient: ethyl acetate/hexanes). Desired product fractions were combined and concentrated to provide compound 310-2 as a viscous oil that semi-solidified under vacuum and was used directly in the next step.

LC/MS m/z (ES+): 334.2 [M+H]+

Step 3: In a 2 dram vial was Intermediate G (200 mg, 1.0 equiv, 428 μmol), compound 310-2 (142 mg, 1.0 equiv, 428 μmol), PdCl₂ (dppf) (62.6 mg, 0.2 equiv, 85.6 μmol), and potassium phosphate, tribasic (182 mg, 70.9 μL, 2.0 equiv, 856 μmol). The vial was purged with N₂ after which nitrogen-degassed DMF/water 3:1 (2.5 mL) was added and reaction was heated to 80° C. for 1 hr. The reaction was then cooled to RT, diluted with DCM and water. The organics were dried and concentrated, dry loaded on silica and purified by flash silica gel chromatography (gradient DCM/0.5% NH₄OH in MeOH). Product fractions were concentrated and combined to provide compound 310 as a single, enantiopure diastereomer.

LC/MS m/z (ES+): 593.3 [M+H]+

¹H NMR (500 MHZ, DMSO) δ 11.29 (s, 1H), 10.97 (s, 1H), 9.15 (q, J=4.7 Hz, 1H), 8.43 (s, 1H), 8.15 (s, 1H), 8.04 (t, J=7.8 Hz, 1H), 7.99 (s, 1H), 7.92 (d, J=7.6 Hz, 1H), 7.82 (dd, J=7.9, 1.1 Hz, 1H), 7.49 (dd, J=7.9, 1.6 Hz, 1H), 7.30 (dd, J=8.0, 1.6 Hz, 1H), 7.20 (t, J=7.9 Hz, 1H), 5.74 (s, 1H), 5.05 (t, J=6.9 Hz, 1H), 3.97-3.89 (m, 2H), 3.79 (td, J=14.3, 13.5, 6.3 Hz, 2H), 3.58 (s, 3H), 3.55 (t, J=8.0 Hz, 1H), 3.39 (dd, J=14.2, 7.2 Hz, 1H), 2.86 (d, J=4.6 Hz, 3H), 2.06 (dd, J=6.8, 4.2 Hz, 1H), 1.08 (t, J=7.0 Hz, 1H), 0.80 (dq, J=5.4, 3.3 Hz, 4H), 0.63 (d, J=6.3 Hz, 3H).

### Example 311

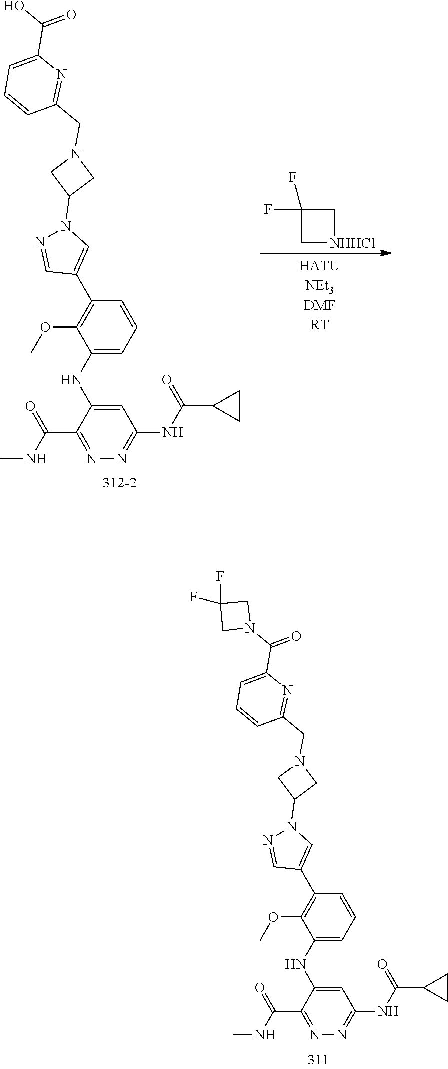

A screw cap vial was loaded with 3,3-difluoroazetidine HCl salt (8.45 mg, 1.3 equiv, 65.3 μmol), acid 312-2 (30.0 mg, 1.0 equiv, 50.2 μmol), HATU (22.9 mg, 1.2 equiv, 60.2 μmol). The vial was filled with N,N-dimethylformamide (1.0 mL) followed by triethylamine (12.7 mg, 17.5 μL, 2.5 equiv, 125 μmol) after which the reaction was stirred at RT for 30 min until the reaction was determined to be complete by LC/MS analysis. The reaction was then diluted to approximately 2.0 mL with DMSO, filtered and purified by high pressure chromatography (C18 column, gradient: MeCN/0.1% formic acid in water). The product containing fractions were concentrated to provide the desired product compound 311 as a white solid.

LC/MS m/z (ES+): 673.4 [M+H]+

TABLE 21
The following compounds were prepared in analogous fashion to compound
311 in Example 311:

| | | LCMS m/z | |
|---|---|---|---|
| Compound | Structure | (ES+) | ¹H NMR |
| 354 | 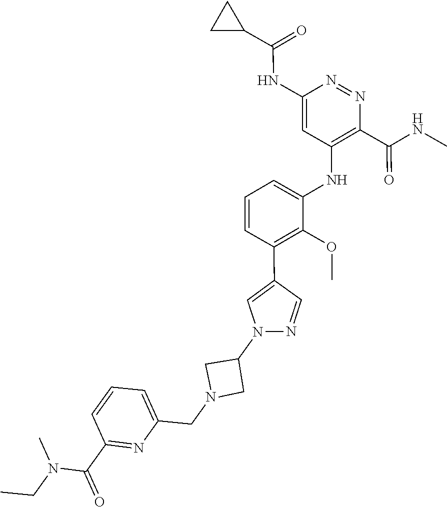 [SMILES: CCN(C)C(=O)c1cccc(CN2CC(n3cc(-c4cccc(Nc5cc(NC(=O)C6CC6)nnc5C(=O)NC)c4OC)cn3)C2)n1] | 639.2 [M + H]+ | |
| 355 | 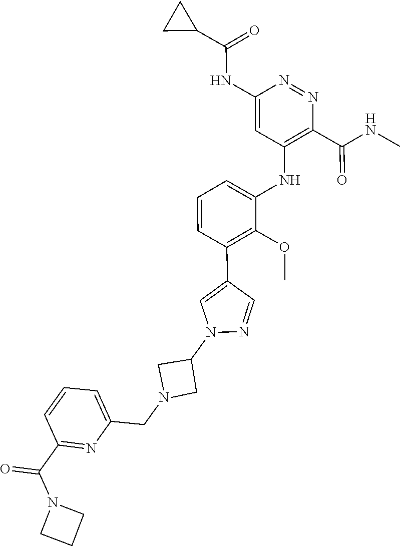 [SMILES: CNC(=O)c1nnc(NC(=O)C2CC2)cc1Nc1cccc(-c2cnn(C3CN(Cc4cccc(C(=O)N5CCC5)n4)C3)c2)c1OC] | 637.3 [M + H]+ | |
| 356 | 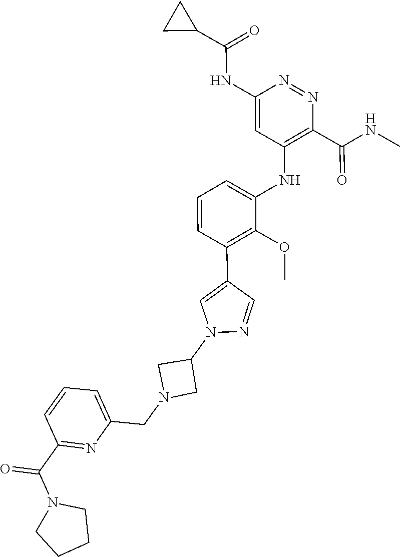 [SMILES: CNC(=O)c1nnc(NC(=O)C2CC2)cc1Nc1cccc(-c2cnn(C3CN(Cc4cccc(C(=O)N5CCCC5)n4)C3)c2)c1OC] | 651.2 [M + H]+ | |
| 357 | 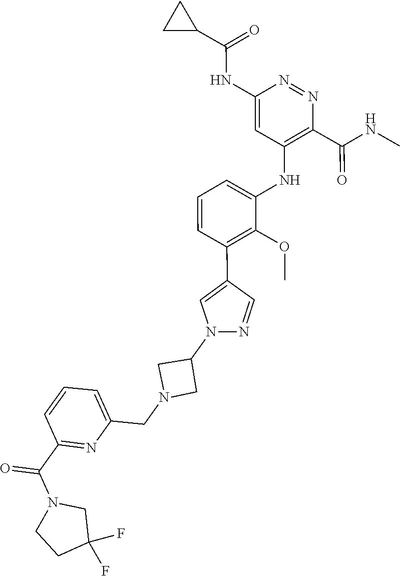 [SMILES: CNC(=O)c1nnc(NC(=O)C2CC2)cc1Nc1cccc(-c2cnn(C3CN(Cc4cccc(C(=O)N5CCC(F)(F)C5)n4)C3)c2)c1OC] | 687.2 [M + H]+ | |
| 358 | 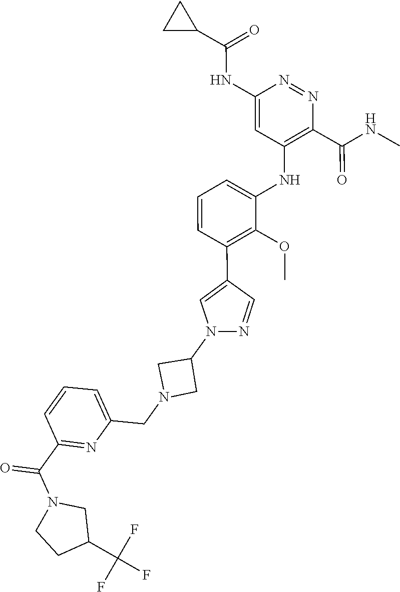 [SMILES: CNC(=O)c1nnc(NC(=O)C2CC2)cc1Nc1cccc(-c2cnn(C3CN(Cc4cccc(C(=O)N5CCC(C(F)(F)F)C5)n4)C3)c2)c1OC] | 719.2 [M + H]+ | |

### Example 312

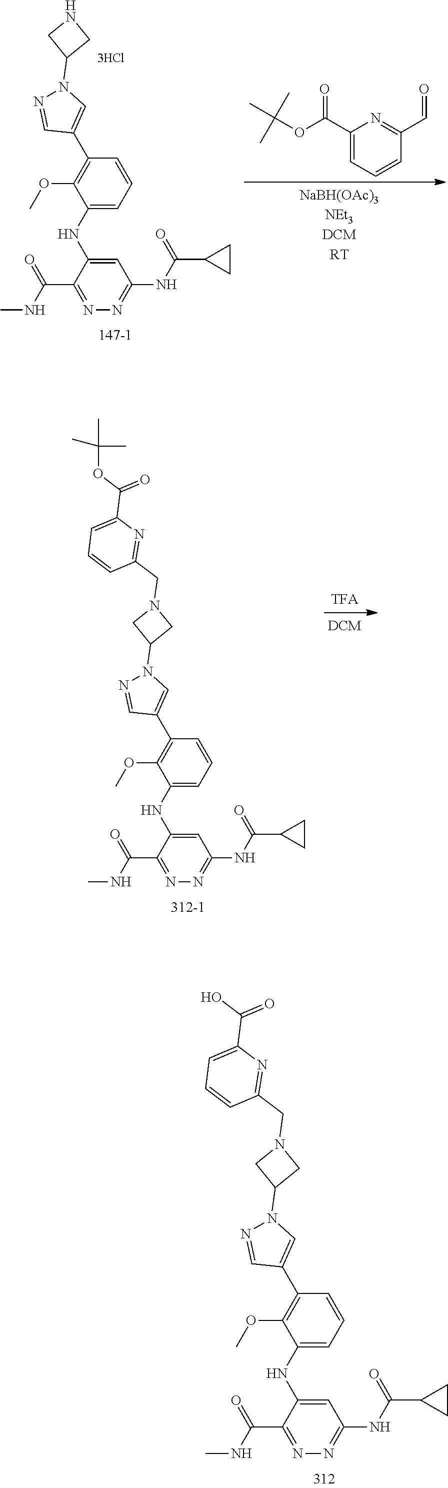 [SMILES: CNC(=O)c1nnc(NC(=O)C2CC2)cc1Nc1cccc(-c2cnn(C3CNC3)c2)c1OC ; CC(C)(C)OC(=O)c1cccc(C=O)n1 ; CNC(=O)c1nnc(NC(=O)C2CC2)cc1Nc1cccc(-c2cnn(C3CN(Cc4cccc(C(=O)OC(C)(C)C)n4)C3)c2)c1OC ; CNC(=O)c1nnc(NC(=O)C2CC2)cc1Nc1cccc(-c2cnn(C3CN(Cc4cccc(C(=O)O)n4)C3)c2)c1OC]

Step 1: A mixture of compound 147-1 (200 mg, 1.0 equiv, 432 μmol), tert-butyl 6-formylpicolinate (89.6 mg, 1.0 equiv, 432 μmol), and triethylamine (87.5 mg, 2.0 equiv, 865 μmol) were stirred in DCM (5 mL) for 20 minutes at RT, followed by the slow addition of sodium triacetoxyhydroborate (367 mg, 4.0 equiv, 1.73 mmol) over 20 minutes. The reaction is stirred at RT for 16 h, after which the reaction was determined to be complete by LC/MS analysis. The reaction is then concentrated, resuspended in DMSO, filtered and purified by high pressure chromatography (C18 column, gradient MeCN/0.1% formic acid in H₂O). The product containing fractions are concentrated to provide compound 312-1 that is used directly in the next step.

Step 2: Ester 312-1 is suspended in 8 mL of a 1:1 DCM:TFA solution and stirred at RT for 30 min, after which the reaction was determined to be complete by LC/MS analysis. The reaction was concentrated, triturated with diethyl ether and dried over high vac to provide the acid 312 as a pale yellow solid.

LC/MS m/z (ES+): 598.4 [M+H]+

¹H NMR (500 MHz, DMSO) δ 11.29 (s, 1H), 10.96 (s, 1H), 9.14 (d, J=4.8 Hz, 1H), 8.37 (s, 1H), 8.14 (d, J=4.6 Hz, 2H), 8.02 (s, 1H), 7.99-7.77 (m, 2H), 7.62 (dd, J=7.6, 1.4 Hz, 1H), 7.47 (dd, J=7.9, 1.6 Hz, 1H), 7.29 (dd, J=7.9, 1.6 Hz, 1H), 7.19 (t, J=7.9 Hz, 1H), 5.12 (p, J=6.9 Hz, 1H), 3.90 (s, 2H), 3.82 (td, J=7.1, 1.7 Hz, 2H), 3.75-3.52 (m, 5H), 2.85 (d, J=4.9 Hz, 3H), 2.07 (tt, J=7.1, 5.3 Hz, 1H), 0.95-0.57 (m, 4H).

### Example 313

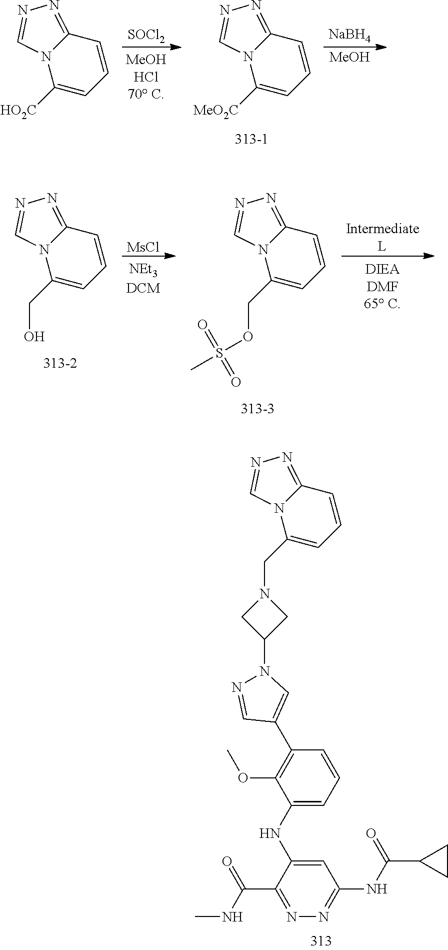

Step 1: To a stirred 0° C. suspension of [1,2,4]triazolo[4,3-a]pyridine-5-carboxylic acid, HCl (0.9987 g, 1.0 equiv, 5.004 mmol) in MeOH (10 mL) was added dropwise thionyl chloride (1.086 g, 666.4 μL, 1.825 equiv, 9.131 mmol). The reaction was stirred at 70° C. for 2 hours. The mixture was then cooled to RT. Unreacted insoluble starting acid was removed by filtration after addition of DCM (30 mL). The filtrate was concentrated down and taken up in DCM (30 mL) and washed with saturated aqueous NaHCO₃ (10 mL). The organic layer was brine-washed, dried over sodium sulfate, filtered, concentrated down, and dried to yield compound 313-1 (141 mg, 796 μmol, 16%) as a brown solid.

LC/MS (ES+): m/z=178.2 [M+H]+.

Step 2: To a stirred RT mixture of methyl [1,2,4]triazolo[4,3-a]pyridine-5-carboxylate (141 mg, 1 Eq, 796 μmol) in MeOH (2 mL) was added carefully sodium tetrahydroborate (151 mg, 5.0 equiv, 3.98 mmol) in 3 portions over 1-2 minutes. The mixture was stirred at 50° C. for 4 hours, cooled to RT, and quenched with water (1 mL). The bulk of solvents was removed under reduced pressure. The residue was diluted with DCM (40 mL) and washed with brine (10 mL). The aqueous layer was extracted with fresh DCM (20 mL). The organic layers were brine-washed, dried over sodium sulfate, filtered, concentrated down, and dried to yield compound 313-2 (52.2 mg, 350 μmol, 44% yield) as a brown solid.

LC/MS (ES+): m/z=150.1 [M+H]+.

Step 3: To a stirred mixture of 313-2 (52.2 mg, 1.0 equiv, 350 μmol), and triethylamine (106 mg, 146 μL, 3.0 equiv, 1.05 mmol) in DCM (1.5 mL) was added dropwise methanesulfonyl chloride (40.1 mg, 27.1 μL, 1.0 equiv, 350 μmol) over several minutes. The reaction solution was stirred for 2 days at R.T. The reaction was diluted with DCM (5 mL) and treated with aqueous saturated NaHCO₃ (5 mL). The layers were separated, and the aqueous layer was extracted with fresh DCM (5 mL). The combined organic layers were dried over sodium sulfate, filtered, concentrated down, and dried to yield compound 313-3 (101.9 mg, 0.36 mmol, 100%, 80% Purity) that was used directly in the next step.

Step 4: To a not fully dissolved mix of Intermediate L (60 mg, 1.0 equiv, 0.13 mmol) and diisopropylethylamine (50 mg, 67 μL, 3.0 equiv, 0.39 mmol) in DMF (0.3 mL) in a sealed tube was added a solution of compound 313-3 and stirred at 65° C. for 2 hours. The reaction was cooled to RT and treated with water (˜ 8 mL). The beige precipitate that formed was isolated by filtration, dried, dissolved in DCM, and purified by silica gel chromatography (gradient: DCM/10:90:0.5 MeOH/DCM/NH₄OH to provide compound 313 (35.0 mg, 58 μmol, 45%, 99% Purity) as a white, foamy solid.

LC/MS (ES+): m/z=594.1 [M+H]⁺.

¹H NMR (500 MHZ, DMSO) δ 11.29 (s, 1H), 10.96 (s, 1H), 9.35 (d, J=0.8 Hz, 1H), 9.15 (q, J=4.8 Hz, 1H), 8.36 (s, 1H), 8.13 (s, 1H), 8.03 (s, 1H), 7.72 (d, J=9.2 Hz, 1H), 7.46 (dd, J=7.8, 1.5 Hz, 1H), 7.36 (dd, J=9.2, 6.6 Hz, 1H), 7.29 (dd, J=8.0, 1.5 Hz, 1H), 7.19 (t, J=7.9 Hz, 1H), 6.98 (d, J=6.7 Hz, 1H), 5.13 (p, J=7.0 Hz, 1H), 4.11 (s, 2H), 3.83 (t, J=7.8 Hz, 2H), 3.65 (t, J=7.5 Hz, 2H), 3.58 (s, 3H), 2.86 (d, J=4.8 Hz, 3H), 2.10-2.03 (m, 1H), 0.83-0.76 (m, 4H).

TABLE 22
The following compounds were prepared using analogous procedures from
Example 313:

| | | LCMS m/z | |
|---|---|---|---|
| Compound | Structure | (ES+) | ¹H NMR |
| 373 | 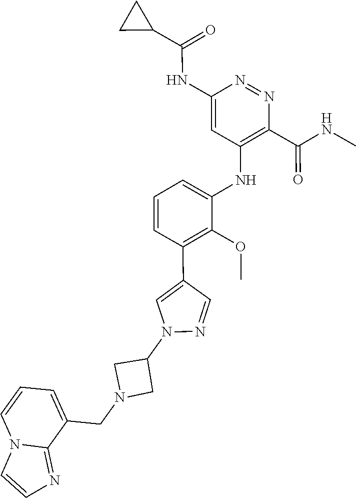 [SMILES: CNC(=O)c1nnc(NC(=O)C2CC2)cc1Nc1cccc(-c2cnn(C3CN(Cc4cccn5ccnc45)C3)c2)c1OC] | 593.2 [M + H]+ | ¹H NMR (500 MHz, DMSO) δ 11.29 (s, 1H), 10.96 (s, 1H), 9.14 (q, J = 4.8 Hz, 1H), 8.44 (dd, J = 6.8, 1.2 Hz, 1H), 8.37 (s, 1H), 8.14 (s, 1H), 8.01 (s, 1H), 7.93 (d, J = 1.2 Hz, 1H), 7.53 (d, J = 1.2 Hz, 1H), 7.47 (dd, J = 7.8, 1.5 Hz, 1H), 7.29 (dd, J = 7.9, 1.6 Hz, 1H), 7.22-7.15 (m, 2H), 6.86 (t, J = 6.8 Hz, 1H), 5.12 (p, J = 6.9 Hz, 1H), 4.05 (s, 2H), 3.84 (t, J = 7.7 Hz, 2H), 3.62 (t, J = 7.4 Hz, 2H), 3.58 (s, 3H), 2.86 (d, J = 4.8 Hz, 3H), 2.11-2.02 (m, 1H), 0.83-0.77 (m, 4H). |
| 374 | 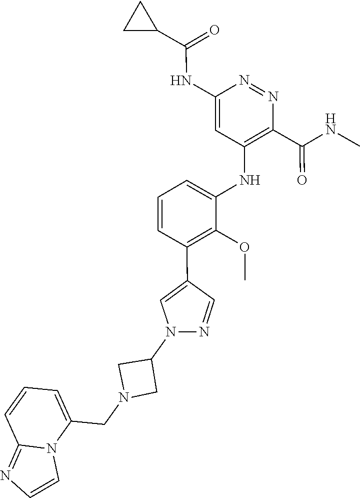 [SMILES: CNC(=O)c1nnc(NC(=O)C2CC2)cc1Nc1cccc(-c2cnn(C3CN(Cc4cccc5nccn45)C3)c2)c1OC] | 593.1 [M + H]+ | ¹H NMR (500 MHz, DMSO) δ 11.29 (s, 1H), 10.97 (s, 1H), 9.15 (q, J = 4.7 Hz, 1H), 8.38 (s, 1H), 8.14 (s, 1H), 8.03 (s, 1H), 7.98- 7.95 (m, 1H), 7.63 (d, J = 1.3 Hz, 1H), 7.53 (d, J = 9.0 Hz, 1H), 7.46 (dd, J = 7.9, 1.6 Hz, 1H), 7.29 (dd, J = 7.9, 1.6 Hz, 1H), 7.23 (dd, J = 9.1, 6.8 Hz, 1H), 7.19 (t, J = 7.9 Hz, 1H), 6.92 (dd, J = 6.8, 1.1 Hz, 1H), 5.13 (p, J = 7.0 Hz, 1H), 4.05 (s, 2H), 3.83 (td, J = 7.1, 1.6 Hz, 2H), 3.64 (td, J = 6.7, 1.6 Hz, 2H), 3.58 (s, 3H), 2.86 (d, J = 4.7 Hz, 3H), 2.11-2.03 (m, 1H), 0.86-0.77 (m, 4H). |

### Example 315

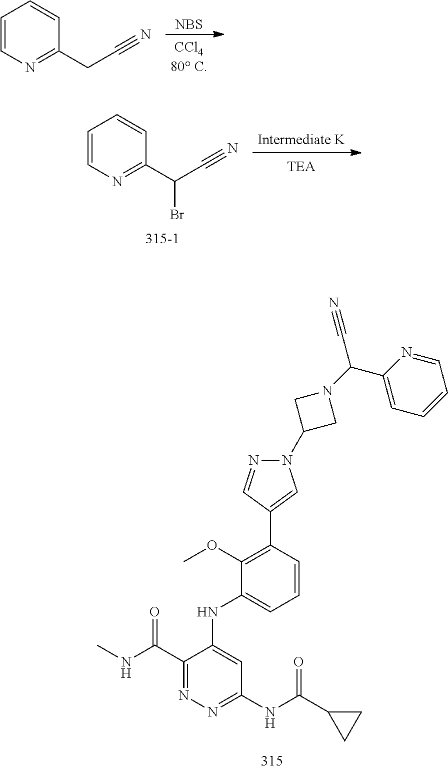

Step 1: A solution of 2-(pyridin-2-yl) acetonitrile (50 mg, 1.0 equiv, 0.42 mmol), NBS (1.0 equiv) in CCl₄ (2 mL) was stirred for 1.5 h at 80° C. until the reaction was determined to be complete by LC/MS analysis. The reaction was then quenched with water and extracted with DCM. The organic layer was washed with brine, dried over Na₂SO₄ and evaporated. The resulting crude material was purified by flash silica gel chromatography to provide compound 315-1 that was used directly in the next step.

m/z (ES+) [M+H]+=197.0

Step 2: A solution of compound 315-1 (10 mg, 1.0 equiv, 51 μmol) and Intermediate K (23 mg, 1.0 equiv, 51 μmol) and TEA in a flask was stirred for 2 h at room temperature until it was determined to be complete by LC/MS analysis. The reaction was quenched with water and extracted with ethyl acetate. The organic layer was washed with brine, dried over Na₂SO₄ and evaporated. The resulting crude material was purified using prep-HPLC to provide racemic compound 315 (9.2 mg, 16 μmol, 31%) as a white amorphous solid.

m/z (ES+) [M+H]+=579.2

¹H NMR (400 MHZ, DMSO-d6) 11.32 (s, 1H), 10.98 (s, 1H), 9.18 (d, J=4.9 Hz, 1H), 8.65-8.58 (m, 1H), 8.37 (s, 1H), 8.16 (s, 1H), 8.06 (s, 1H), 7.91 (td, J=7.7, 1.8 Hz, 1H), 7.54 (d, J=7.9 Hz, 1H), 7.51-7.41 (m, 2H), 7.31 (dd, J=7.9, 1.6 Hz, 1H), 7.21 (t, J=7.9 Hz, 1H), 5.52 (s, 1H), 5.20 (p, J=6.9 Hz, 1H), 3.88 (dt, J=20.2, 7.1 Hz, 2H), 3.82-3.71 (m, 2H), 3.60 (s, 3H), 2.87 (d, J=4.8 Hz, 3H), 2.08 (q, J=6.2 Hz, 1H), 0.86-0.79 (m, 4H).

### Example 318

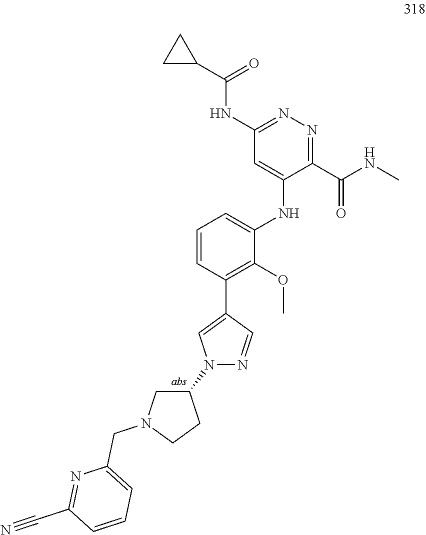 [SMILES: CNC(=O)c1nnc(NC(=O)C2CC2)cc1Nc1cccc(-c2cnn([C@@H]3CCN(Cc4cccc(C#N)n4)C3)c2)c1OC]

Compound 318 was prepared in analogous fashion to the procedures in Example 147 except that tert-butyl-(4-(4,4,5,5-tetramethyl-1,3,2-dioxaborolan-2-yl)-1H-pyrazol-1-yl) pyrrolidine-1-carboxylate was used in place of tert-butyl 3-(4-(4,4,5,5-tetramethyl-1,3,2-dioxaborolan-2-yl)-1H-pyrazol-1-yl) azetidine-1-carboxylate. The enantiomers were separated under the following conditions: Column: CHIRALPAK IC, 2*25 cm, 5 um; Mobile Phase A: Hex (0.1% IPAmine)-HPLC, Mobile Phase B: EtOH:DCM=1:1-HPLC; Flow rate: 20 mL/min; Gradient: 50% B to 50% B in 14.5 min; Wave Length: 220/254 nm; RT1 (min): 7.88; RT2 (min): 12.713

m/z (ES+) [M+Na]+=615.3

¹H NMR (Chloroform-d, 400 MHZ) 11.17 (1H, s), 8.96 (1H, s), 8.30 (1H, s), 8.16 (1H, d, J=5.3 Hz), 8.07 (1H, s), 7.94 (3H, d, J=3.8 Hz), 7.68 (1H, d, J=7.4 Hz), 7.37 (1H, dd, J=7.8, 1.6 Hz), 7.32 (1H, dd, J=7.9, 1.6 Hz), 7.22 (1H, t, J=7.9 Hz), 5.06 (1H, s), 4.50-4.02 (2H, m), 3.65 (3H, s), 3.45-3.15 (2H, s), 3.08 (3H, d, J=5.1 Hz), 2.65 (1H, dd, J=14.1, 7.9 Hz), 2.34 (1H, td, J=8.3, 3.7 Hz), 1.72 (2H, tt, J=7.9, 4.4 Hz), 1.13 (2H, dt, J=6.7, 3.4 Hz), 0.96 (2H, dq, J=7.6, 4.1 Hz), 0.86 (1H, s).

### Example 319

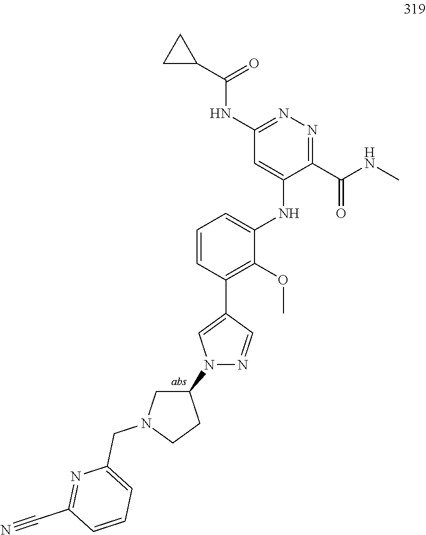 [SMILES: CNC(=O)c1nnc(NC(=O)C2CC2)cc1Nc1cccc(-c2cnn([C@H]3CCN(Cc4cccc(C#N)n4)C3)c2)c1OC]

Compound 319 was isolated as the 2^{nd} enantiomer from chiral HPLC purification from the racemic mixture in Example 147. The enantiomers were separated under the following conditions: Column: CHIRALPAK IC, 2*25 cm, 5 um; Mobile Phase A: Hex (0.1% IPAmine)-HPLC, Mobile Phase B: EtOH:DCM=1:1-HPLC; Flow rate: 20 mL/min; Gradient: 50% B to 50% B in 14.5 min; Wave Length: 220/254 nm; RT1 (min): 7.88; RT2 (min): 12.713

m/z (ES+) [M+Na]+=615.3

¹H NMR (Chloroform-d, 400 MHz) 11.17 (1H, s), 8.89 (1H, s), 8.30 (1H, s), 8.17 (1H, d, J=5.3 Hz), 8.08 (1H, s), 7.93 (3H, d, J=3.6 Hz), 7.67 (1H, d, J=7.5 Hz), 7.37 (1H, dd, J=7.9, 1.6 Hz), 7.32 (1H, dd, J=7.8, 1.6 Hz), 7.22 (1H, t, J=7.9 Hz), 5.06 (1H, s), 4.50-4.02 (2H, s), 3.65 (3H, s), 3.45-3.15 (2H, s), 3.08 (3H, d, J=5.1 Hz), 2.64 (1H, dd, J=14.1, 7.5 Hz), 2.34 (1H, ddd, J=13.2, 8.6, 4.3 Hz), 2.03 (1H, s), 1.71 (2H, td, J=7.9, 4.0 Hz), 1.17-1.09 (2H, m), 0.96 (2H, dq, J=7.5, 4.1 Hz).

### Example 320

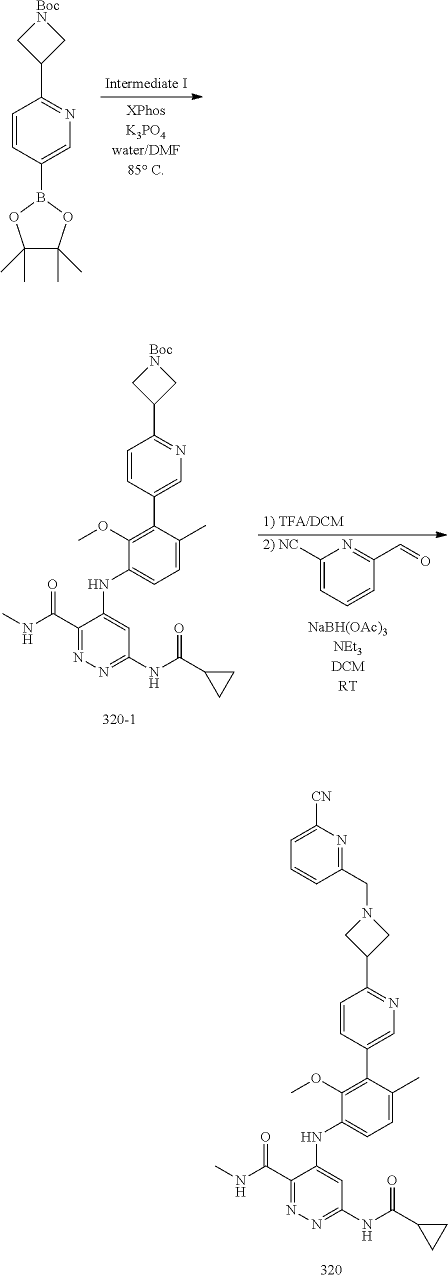

Step 1: A screw cap vial was loaded with tert-butyl 3-(5-(4,4,5,5-tetramethyl-1,3,2-dioxaborolan-2-yl) pyridin-2-yl) azetidine-1-carboxylate (402.4 mg, 1.3 equiv, 1.117 mmol), Intermediate I (373.2 mg, 1.0 equiv, 859.2 μmol), methanesulfonato (2-dicyclohexylphosphino-2′,4′,6′-tri-i-propyl-1,1′-biphenyl) (2′-methylamino-1,1′-biphenyl-2-yl) palladium (II) (111.0 mg, 0.15 equiv, 128.9 μmol) and potassium phosphate, dibasic (299.3 mg, 2.0 equiv, 1.718 mmol). The vial was purged with N₂, filled with Water (1.4 mL) and N,N-dimethylformamide (7.0 mL) and heated at 85° C. for 16 h until LC/MS analysis showed about 50% conversion to desired product. The reaction is cooled down, diluted in sat NH₄Cl solution and EtOAC and extracted with water and brine. The organic layer is dried over Na₂SO₄, concentrated and the residue is purified by flash silica gel chromatography on silica gel (gradient: MeOH:DCM 0-10%+0.1% NH₄OH). The product containing fractions are concentrated yielding compound 320-1 as a white solid which was used directly in the next step.

Step 2: Compound 320-1 was resuspended in 4 mL of 1:1 DCM:TFA. After 5 minutes, LC/MS analysis indicated full conversion of starting material towards desired product. The reaction was concentrated yielding the desired product as a glassy solid, the crude mixture was triturated with ethyl ether, dried under vacuum and used in the next step without further purification.

LC/MS (ES+): m/z=488.3 [M+H]+

A mixture of deprotected azetidine (50 mg, 1.0 equiv, 0.10 mmol), 6-cyanopicolinaldehyde (11 mg, 1.0 equiv, 0.10 mmol) and triethylamine (21 mg, 2.0 equiv, 0.21 mmol) were stirred in DCM (5 mL) for 20 minutes at RT followed by the slow addition of sodium triacetoxyhydroborate (87 mg, 4.0 equiv, 0.41 mmol) over 20 minutes. The reaction is stirred at RT for 16 h, after which it was determined to be complete by LC/MS analysis. The reaction is concentrated, resuspended in DMSO, filtered and purified by high pressure chromatography on C18 (Accq prep, gradient: MeCN/0.1% formic acid in water) to provide compound 320.

LC/MS (ES+): m/z=604.3 [M+H]+

TABLE 23
The following compounds were prepared using analogous procedures as
those used for the formation of Example 320.

| | | LCMS m/z | |
|---|---|---|---|
| Compound | Structure | (ES+) | ¹H NMR |
| 321 | 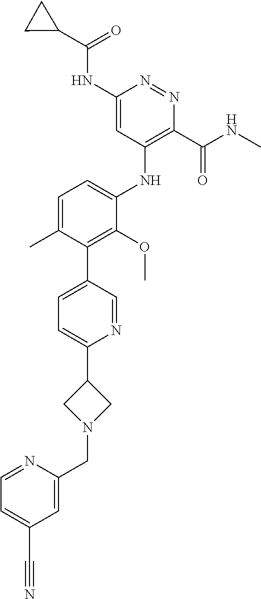 [SMILES: CNC(=O)c1nnc(NC(=O)C2CC2)cc1Nc1ccc(C)c(-c2ccc(C3CN(Cc4cc(C#N)ccn4)C3)nc2)c1OC] | 604.4 [M + H]+ | |
| 322 | 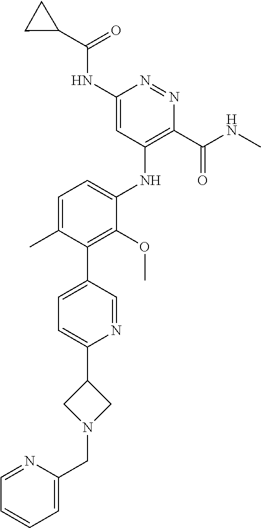 [SMILES: CNC(=O)c1nnc(NC(=O)C2CC2)cc1Nc1ccc(C)c(-c2ccc(C3CN(Cc4ccccn4)C3)nc2)c1OC] | 579.4 [M + H]+ | ¹H NMR (500 MHz, DMSO) δ 11.30 (s, 1H), 10.79 (s, 1H), 9.12 (q, J = 4.8 Hz, 1H), 8.49 (dt, J = 4.6, 1.6 Hz, 1H), 8.47 (d, J = 2.1 Hz, 1H), 8.14 (s, 1H), 8.12 (s, 1H), 7.76 (td, J = 7.7, 1.8 Hz, 1H), 7.71 (dd, J = 7.9, 2.3 Hz, 1H), 7.48 (d, J = 7.9 Hz, 1H), 7.41 (d, J = 7.8 Hz, 1H), 7.38 (d, J = 8.2 Hz, 1H), 7.26 (ddd, J = 7.5, 4.8, 1.2 Hz, 1H), 7.18 (d, J = 8.3 Hz, 1H), 3.89 (p, J = 7.4 Hz, 1H), 3.80 (s, 2H), 3.75 (t, J = 7.4 Hz, 2H), 3.50 (t, J = 7.0 Hz, 2H), 3.29 (s, 3H), 2.84 (d, J = 4.8 Hz, 3H), 2.14-1.98 (m, 4H), 0.92- 0.75 (m, 4H) (formic acid salt) |

### Example 323

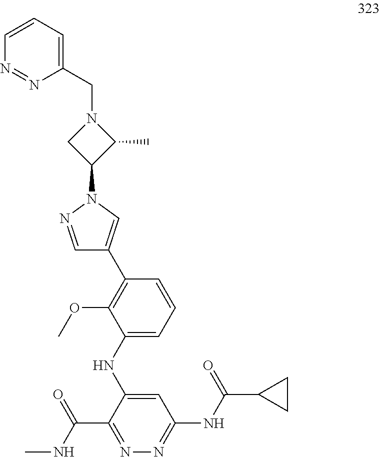 [SMILES: CNC(=O)c1nnc(NC(=O)C2CC2)cc1Nc1cccc(-c2cnn([C@H]3CN(Cc4cccnn4)[C@@H]3C)c2)c1OC]

Compound 323 was prepared using analogous procedures to those in Example 310 except that pyridazine-3-carbaldehyde was used in place of 6-formylpicolinonitrile.

¹H NMR (500 MHz, DMSO) δ 11.29 (s, 1H), 10.96 (s, 1H), 9.17-9.10 (m, 2H), 8.41 (d, J=0.7 Hz, 1H), 8.15 (s, 1H), 7.98 (d, J=0.7 Hz, 1H), 7.74 (dd, J=8.5, 1.7 Hz, 1H), 7.67 (dd, J=8.4, 4.9 Hz, 1H), 7.49 (dd, J=7.8, 1.6 Hz, 1H), 7.29 (dd, J=7.9, 1.6 Hz, 1H), 7.20 (t, J=7.9 Hz, 1H), 5.74 (s, 1H), 5.09-5.02 (m, 1H), 4.05 (d, J=13.8 Hz, 1H), 3.93 (d, J=13.7 Hz, 1H), 3.88 (d, J=8.7 Hz, 1H), 3.82 (q, J=6.5 Hz, 1H), 3.63-3.56 (m, 1H), 3.57 (s, 3H), 3.42 (s, 2H), 2.86 (d, J=4.8 Hz, 3H), 2.11-2.02 (m, OH), 1.08 (t, J=7.0 Hz, 1H), 0.80 (dq, J=5.5, 3.2 Hz, 4H), 0.59 (d, J=6.3 Hz, 3H).

### Example 328

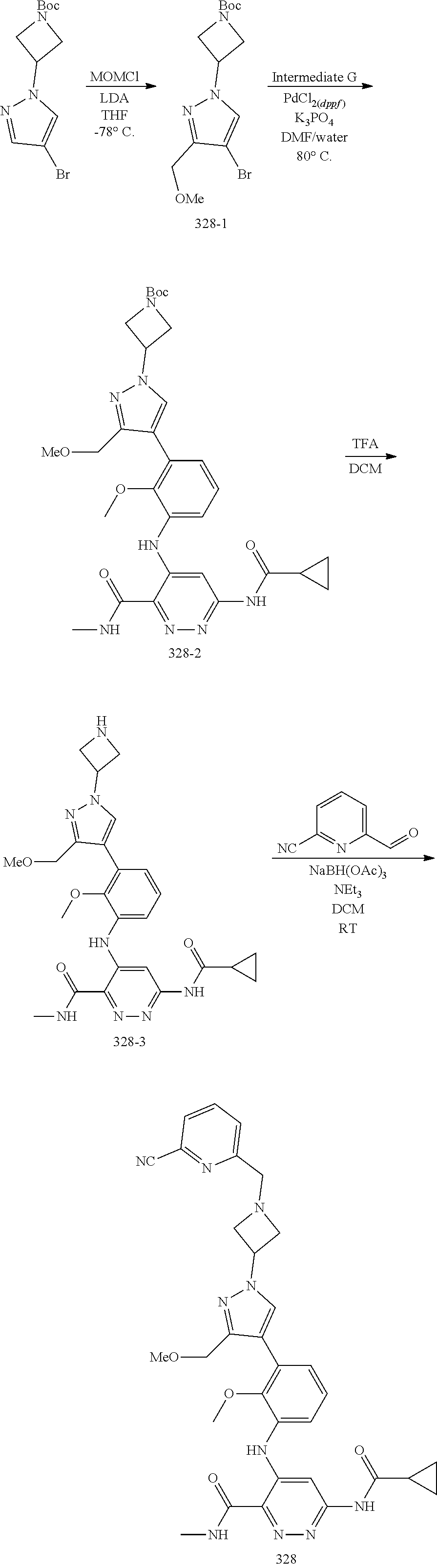

Step 1: In a 40 mL pressure vial was charged tert-butyl 3-(4-bromo-1H-pyrazol-1-yl) azetidine-1-carboxylate (504 mg, 1.0 equiv, 1.67 mmol) in 8 mL of anhydrous THF. The reaction was then cooled to −78° C. after which LDA was added (2.50 mL, 1 molar, 1.5 equiv, 2.50 mmol) and reaction was stirred for 1 hr. Chloromethyl methyl ether (134 mg, 126 μL, 1.0 equiv, 1.67 mmol) was then added in 1 mL of THF and reaction gradually warmed to rt and stirred for 45 min. The reaction was quenched with aq. NH₄Cl and extracted with EtOAc. The organics were washed with water, brine dried and concentrated product purified by flash silica column chromatography (gradient: ethyl acetate/hexanes) to provide 328-1 which as used directly in the next step.

Step 2: Compound 328-1 (400 mg) with Intermediate G (541 mg, 0.69 equiv, 1.16 mmol), K₃PO₄ (1.8 equiv) and Pd(ddpf)Cl₂ (0.15 equiv) was charged in a 30 mL vial, after which it was evacuated purged with N₂ followed by the addition of nitrogen-degassed DMF/water (5 mL, 3:1 v/v) and the reaction heated to 80° C. for 5 hrs until complete conversion was observed by LC/MS analysis. The reaction was then diluted with DCM and water. The organics were washed with water, brine, dried and concentrated. The product was purified by flash silica gel column chromatography (gradient: DCM/MeOH) to provide compound 328-2 (500 mg) which was used directly in the next step.

Step 3: Compound 328-3 was stirred with DCM/TFA (10 mL) stirred for 1 hr. The reaction was then concentrated and purified by flash silica gel column chromatography (gradient: DCM/MeOH). The product fractions combined and concentrated provide an off white solid as compound 328-3.

LC/MS (ES+): m/z=507.2 [M+H]+

Step 4: Compound 328-3 and 6-formyl-2-pyridinecarbonitrile (61 mg, 0.28 equiv, 0.46 mmol) was dissolved in 2 mL of DCM and added TEA (169 mg, 232 μL, 1.0 equiv, 1.67 mmol), stirred for 10 min followed by the addition of sodium triacetoxyborohydride (354 mg, 1.0 equiv, 1.67 mmol) and stirred for 18 hrs until the reaction was determined to be complete by LC/MS analysis. The reaction was then diluted with DCM/water and the organics were dry loaded on silica and purified by flash silica gel column chromatography (gradient: DCM/MeOH) to provide compound 328 as an off-white solid.

LC/MS (ES+): m/z=623.2 [M+H]+

¹H NMR (400 MHZ, DMSO) δ 11.30 (s, 1H), 10.92 (s, 1H), 9.14 (q, J=4.8 Hz, 1H), 8.14 (s, 1H), 7.72 (s, 1H), 7.39 (dd, J=8.0, 1.6 Hz, 1H), 7.23 (t, J=7.9 Hz, 1H), 7.08 (dd, J=7.7, 1.6 Hz, 1H), 5.34 (p, J=7.7 Hz, 1H), 4.41 (s, 2H), 4.10 (td, J=7.4, 1.6 Hz, 2H), 3.85-3.71 (m, 2H), 3.35 (s, 3H), 3.20 (s, 3H), 2.84 (d, J=4.8 Hz, 3H), 2.07 (tt, J=6.9, 5.4 Hz, 1H), 1.00 (s, 2H), 0.82 (h, J=3.2 Hz, 4H).

### Example 329

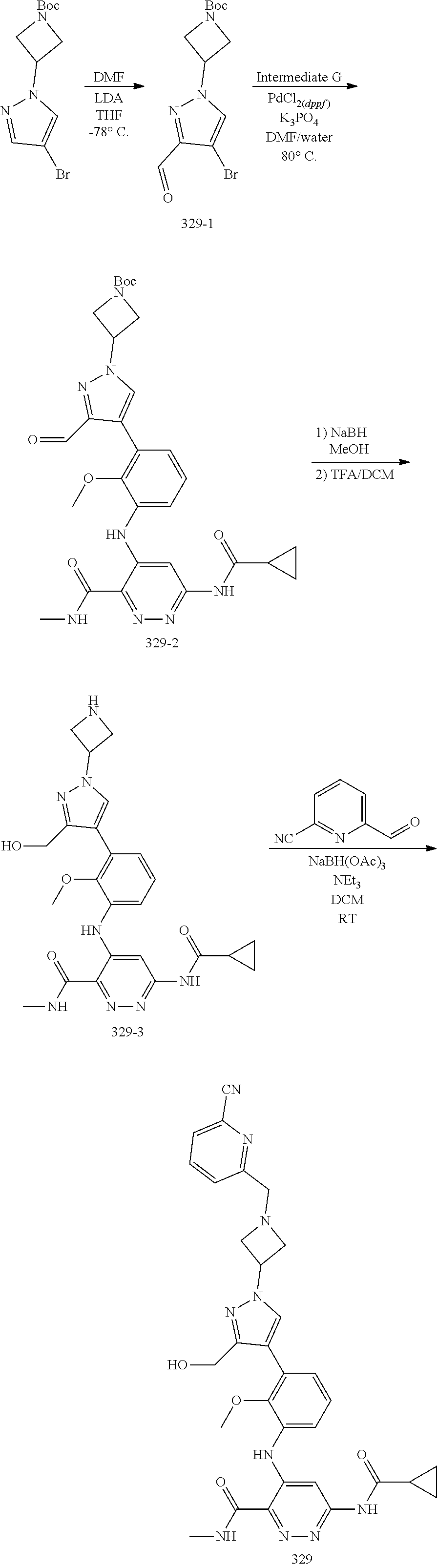

Step 1: In a 40 ml pressure vial was charged tert-butyl 3-(4-bromo-1H-pyrazol-1-yl) azetidine-1-carboxylate (1500 mg, 1.0 equiv, 4.964 mmol) in 16 mL of anhydrous THF. The reaction was cooled to −78° C. after which LDA was added (7.446 mL, 1 molar, 1.5 equiv, 7.446 mmol) and the reaction was stirred for 1 hr. DMF (1.09 g, 1.15 mL, 3.0 equiv, 14.89 mmol) was added in 1 mL of THF and the reaction was gradually warmed to rt and stirred for 45 min. The reaction was quenched with aq. NH₄Cl and extracted with EtOAc. The organics were washed with water, brine dried and concentrated product was purified by flash silica gel column chromatography (gradient: hexanes/ethyl acetate) to provide compound 329-1 as a viscous oil.

Step 2: Compound 329-1 (1340 mg) with Intermediate G (1.856 g, 0.8 equiv, 3.971 mmol), K₃PO₄ (1.8 equiv) and Pd(ddpf)Cl₂ (0.15 equiv) was charged in a 30 mL vial, evacuated and purged with nitrogen after which nitrogen-degassed DMF/water (12 mL, 3:1 v/v) and the reaction was heated to 80° C. for 5 hrs until complete conversation was observed by LC/MS analysis. The reaction was then diluted with DCM and water. The organics were washed with water, brine, dried and concentrated. The crude material was purified by flash silica gel chromatography (gradient: MeOH/DCM). Compound 329-2 was isolated as a light brown solid (2000 mg).

LC/MS (ES+): m/z=591.2 [M+H]+

Step 3: In a 40 mL vial was charged compound 329-2 (325 mg, 1.0 equiv, 550 μmol) in 5 mL of MeOH was added NaBH₄ (62.4 mg, 3.0 equiv, 1.65 mmol), after which the reaction turned dark, with continued stirring for 30 min after which the reaction was determined to be complete by LC/MS analysis. The reaction was quenched with brine, extracted with DCM. The organics were dried and concentrated. The crude product was then taken in 1:1 DCM/TFA and stirred at 45° C. for 1 hr. The reaction was concentrated and dry loaded on silica and purified by flash silica gel column chromatography (gradient: MeOH/DCM). The product fractions were combined and concentrated to a light brown oil to provide compound 329-3.

LC/MS (ES+): m/z=493.1 [M+H]+

Step 4: Compound 329-3 was converted to compound 329 using analogous reductive amination procedures in Example 328.

¹H NMR (500 MHZ, DMSO) δ 11.30 (s, 1H), 10.96 (s, 1H), 9.13 (d, J=4.9 Hz, 1H), 8.49 (s, 1H), 8.19 (s, 1H), 8.04 (t, J=7.8 Hz, 1H), 7.92 (dd, J=7.7, 1.1 Hz, 1H), 7.76 (dd, J=8.0, 1.1 Hz, 1H), 7.69 (s, 1H), 7.38 (dd, J=7.9, 1.6 Hz, 1H), 7.22 (t, J=7.8 Hz, 1H), 7.16 (dd, J=7.8, 1.7 Hz, 1H), 5.74 (s, 1H), 5.30-5.21 (m, 2H), 4.48 (d, J=4.9 Hz, 2H), 3.88 (s, 2H), 3.82 (t, J=7.3 Hz, 2H), 3.66 (t, J=7.4 Hz, 2H), 3.38 (s, 3H), 3.16 (d, J=5.1 Hz, 1H), 3.08 (s, 3H), 2.84 (d, J=4.8 Hz, 3H), 2.08 (p, J=6.4 Hz, 1H), 2.06 (s, 1H), 1.23 (s, 1H), 0.85-0.80 (m, 4H).

### Example 331

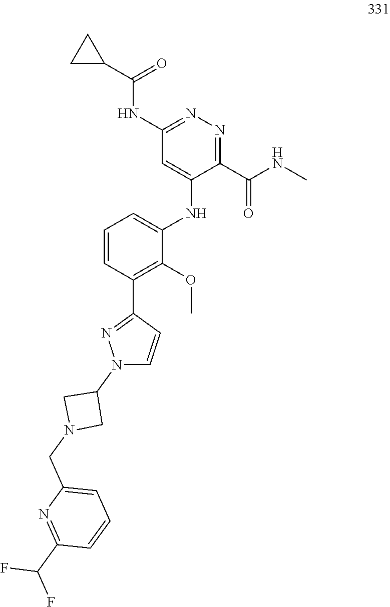 [SMILES: CNC(=O)c1nnc(NC(=O)C2CC2)cc1Nc1cccc(-c2ccn(C3CN(Cc4cccc(C(F)F)n4)C3)n2)c1OC]

Compound 331 was prepared using analogue procedures from Example 341 except that 3-bromo-1H-pyrazole is used in place 4-bromo-1H-imidazole.

LC/MS (ES+): m/z=604.3 M+H]+

¹H NMR (400 MHZ, DMSO-d6) 11.33 (s, 1H), 10.99 (s, 1H), 9.18 (q, J=4.7 Hz, 1H), 8.16 (d, J=10.8 Hz, 1H), 8.04-7.96 (m, 2H), 7.73 (dd, J=7.9, 1.6 Hz, 1H), 7.61 (t, J=7.2 Hz, 2H), 7.41 (dd, J=8.0, 1.6 Hz, 1H), 7.25 (t, J=7.9 Hz, 1H), 6.94 (t, J=55.0 Hz, 1H), 6.78 (d, J=2.2 Hz, 1H), 5.18-5.10 (m, 1H), 3.93 (s, 2H), 3.86 (t, J=7.2 Hz, 2H), 3.65 (t, J=7.0 Hz, 2H), 3.62 (s, 3H), 2.87 (d, J=4.7 Hz, 3H), 2.14-2.03 (m, 1H), 0.87-0.80 (m, 4H).

### Example 333

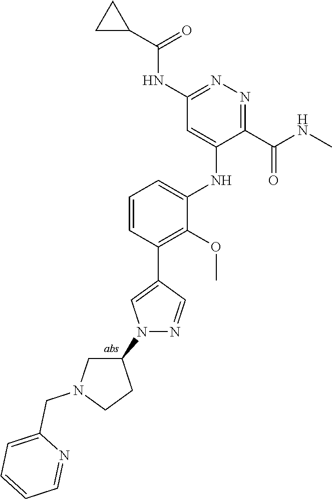 [SMILES: CNC(=O)c1nnc(NC(=O)C2CC2)cc1Nc1cccc(-c2cnn([C@H]3CCN(Cc4ccccn4)C3)c2)c1OC]

Compound 333 was prepared using analogous conditions from Example 318. The racemic mixture was separated to provide compound 333 and compound 334 as enantiompure material (absolute stereochemistry arbitrarily assigned).

m/z (ES+) [M+H]+=568.3

¹H NMR (Chloroform-d, 400 MHz) 11.15 (1H, s), 8.97 (1H, s), 8.63-8.57 (1H, m), 8.30 (1H, s), 8.17 (1H, d, J=5.2 Hz), 8.08 (1H, s), 7.92 (1H, s), 7.79-7.70 (1H, m), 7.57 (1H, s), 7.36 (1H, dd, J=7.9, 1.6 Hz), 7.31 (1H, dd, J=7.8, 1.6 Hz), 7.28-7.17 (2H, m), 5.04 (1H, s), 4.07 (2H, s), 3.65 (3H, s), 3.08 (6H, d, J=5.1 Hz), 2.62 (1H, s), 2.32 (1H, s), 1.73 (2H, tt, J=8.1, 4.5 Hz), 1.13 (2H, dt, J=6.8, 3.5 Hz), 0.95 (2H, dq, J=7.6, 4.1 Hz).

### Example 334

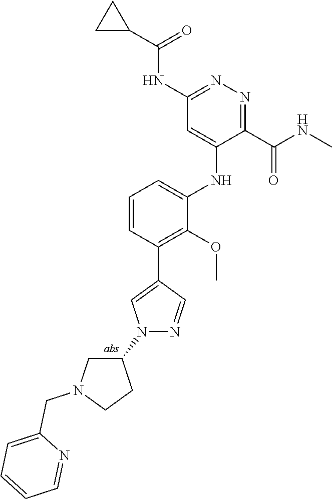 [SMILES: CNC(=O)c1nnc(NC(=O)C2CC2)cc1Nc1cccc(-c2cnn([C@@H]3CCN(Cc4ccccn4)C3)c2)c1OC]

Compound 334 was isolated as the second enantiomer from chiral HPLC separation of the racemic mixture from Example 333.

m/z (ES+) [M+H]+=568.3

¹H NMR (Chloroform-d, 400 MHz) 11.15 (1H, s), 8.95 (1H, s), 8.60 (1H, d, J=4.9 Hz), 8.30 (1H, s), 8.16 (1H, d, J=5.3 Hz), 8.09 (1H, s), 7.91 (1H, s), 7.73 (1H, t, J=7.3 Hz), 7.55 (1H, s), 7.36 (1H, dd, J=7.9, 1.5 Hz), 7.31 (1H, dd, J=7.9, 1.5 Hz), 7.22 (2H, q, J=8.0, 6.5 Hz), 5.02 (1H, s), 4.02 (2H, s), 3.65 (3H, s), 3.37-3.12 (2H, m), 3.08 (3H, d, J=5.0 Hz), 2.91 (1H, s), 2.60 (1H, s), 2.30 (1H, s), 1.72 (2H, td, J=7.9, 4.1 Hz), 1.14 (2H, p, J=4.3 Hz), 0.95 (2H, dq, J=7.6, 4.1 Hz).

### Example 335

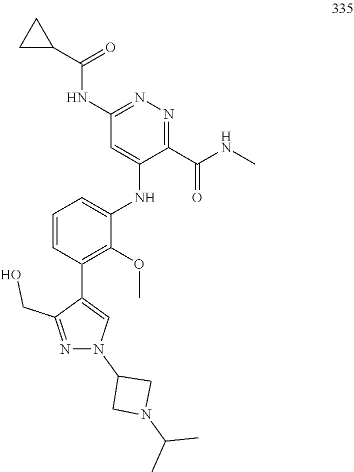 [SMILES: CNC(=O)c1nnc(NC(=O)C2CC2)cc1Nc1cccc(-c2cn(C3CN(C(C)C)C3)nc2CO)c1OC]

Compound 335 was prepared in analogous fashion using the procedures from Example 329 except that acetone was used in place of 6-cyanopicolinaldehyde.

¹H NMR (500 MHz, DMSO) δ 11.31 (s, 1H), 10.96 (s, 1H), 10.29 (s, 1H), 9.15 (q, J=4.8 Hz, 1H), 8.19 (s, 1H), 7.81 (s, 1H), 7.41 (dd, J=8.0, 1.6 Hz, 1H), 7.24 (t, J=7.8 Hz, 1H), 7.15 (dd, J=7.7, 1.6 Hz, 1H), 5.38 (s, 1H), 4.52 (d, J=4.0 Hz, 2H), 4.43 (s, 4H), 3.39 (s, 3H), 2.84 (d, J=4.9 Hz, 3H), 2.12 (s, 4H), 2.09 (dt, J=8.0, 5.0 Hz, 1H), 2.06 (s, 4H), 1.19 (s, 1H), 1.15 (s, 5H), 0.82 (dq, J=9.8, 3.3 Hz, 4H).

TABLE 24
The following compounds were prepared according to analogous procedures
for Example 335:

| | | LCMS m/z | |
|---|---|---|---|
| Compound | Structure | (ES+) | ¹H NMR |
| 336 | 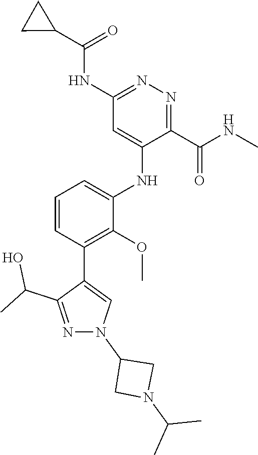 [SMILES: CNC(=O)c1nnc(NC(=O)C2CC2)cc1Nc1cccc(-c2cn(C3CN(C(C)C)C3)nc2C(C)O)c1OC] | 549.2 [M + H]+ | ¹H NMR (500 MHz, DMSO) δ 11.29 (s, 1H), 10.93 (s, 1H), 9.13 (q, J = 4.8 Hz, 1H), 8.26 (s, 1H), 8.16 (s, 1H), 7.54 (s, 1H), 7.39 (dd, J = 8.0, 1.6 Hz, 1H), 7.20 (t, J = 7.9 Hz, 1H), 7.05 (dd, J = 7.7, 1.6 Hz, 1H), 5.41 (p, J = 7.4 Hz, 1H), 4.87 (q, J = 6.7 Hz, 1H), 3.72 (td, J = 6.8, 2.1 Hz, 1H), 3.64 (td, J = 6.8, 2.1 Hz, 1H), 3.49 (t, J = 7.0 Hz, 1H), 3.36 (s, 3H), 3.16 (s, 2H), 2.84 (d, J = 4.8 Hz, 3H), 2.42 (h, J = 6.2 Hz, 1H), 2.12-2.03 (m, 1H), 2.06 (s, 1H), 1.37 (d, J = 6.7 Hz, 3H), 0.91 (d, J = 6.1 Hz, 6H), 0.85-0.79 (m, 4H). |
| 338 | 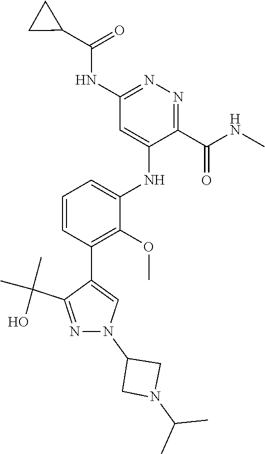 [SMILES: CNC(=O)c1nnc(NC(=O)C2CC2)cc1Nc1cccc(-c2cn(C3CN(C(C)C)C3)nc2C(C)(C)O)c1OC] | 563.1 [M + H]+ | ¹H NMR (500 MHz, DMSO) δ 11.29 (s, 1H), 10.89 (s, 1H), 9.13 (q, J = 4.8 Hz, 1H), 8.44 (s, 5H), 8.13 (s, 1H), 7.40 (dd, J = 8.0, 1.6 Hz, 1H), 7.34 (s, 1H), 7.16 (t, J = 7.8 Hz, 1H), 6.96 (dd, J = 7.6, 1.5 Hz, 1H), 5.78-5.69 (m, 1H), 5.40 (s, 1H), 3.64 (d, J = 13.6 Hz, 1H), 3.64 (s, 1H), 3.42 (t, J = 7.2 Hz, 2H), 3.16 (s, 2H), 2.83 (d, J = 4.7 Hz, 3H), 2.44- 2.37 (m, 1H), 2.12-2.03 (m, 1H), 1.29 (s, 6H), 1.23 (s, 1H), 0.90 (d, J = 6.1 Hz, 6H), |

### Example 337

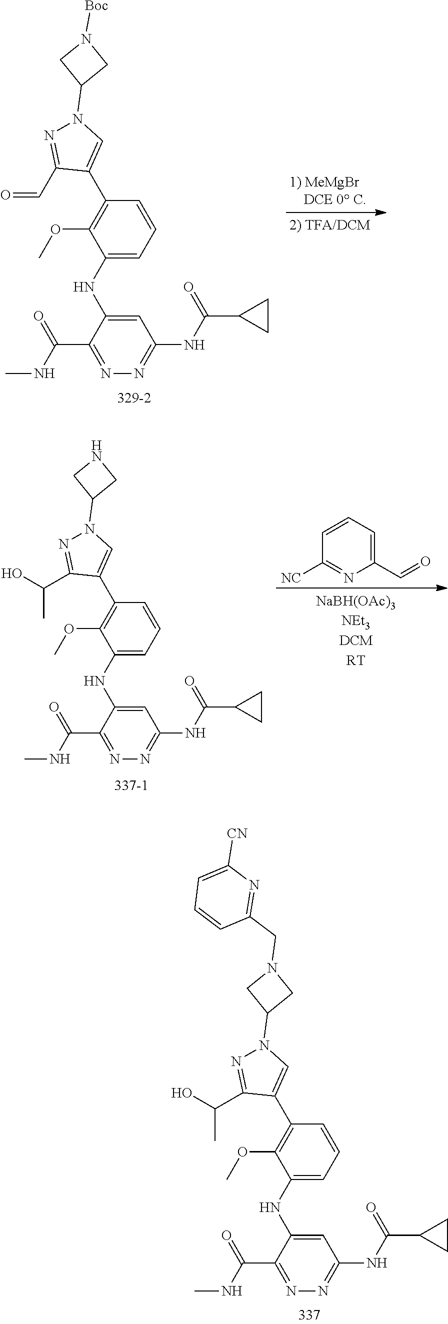 [SMILES: CNC(=O)c1nnc(NC(=O)C2CC2)cc1Nc1cccc(-c2cn(C3CN(C(=O)OC(C)(C)C)C3)nc2C=O)c1OC ; CNC(=O)c1nnc(NC(=O)C2CC2)cc1Nc1cccc(-c2cn(C3CNC3)nc2C(C)O)c1OC ; N#Cc1cccc(C=O)n1 ; CNC(=O)c1nnc(NC(=O)C2CC2)cc1Nc1cccc(-c2cn(C3CN(Cc4cccc(C#N)n4)C3)nc2C(C)O)c1OC]

Step 1: In a 40 mL vial was charged compound 329-2 (850 mg, 1.0 equiv, 1.44 mmol) and magnesium bromide diethyl etherate (743 mg, 2.0 equiv, 2.88 mmol) in 20 mL of DCE. The reaction was then cooled to 0° C. after which additional methylmagnesium bromide (601 mg, 1.68 mL, 3.0 molar, 3.5 equiv, 5.04 mmol) was added and the reaction was stirred for 30 min until the reaction was determined to be complete by LC/MS analysis. The reaction was then quenched with aq NH₄Cl, then partitioned between MTBE and water. The organics were dried and concentrated. Crude material was then dissolved in 1:1 DCE/TFA and stirred at 40° C. for 1 h, after which it was concentrated and purified by flash silica gel chromatography (gradient: MeOH/DCM) to provide compound 337-1.

m/z (ES+) [M+H]+=507.2

Step 2: Compound 337-1 was converted to compound 337 using the analogue procedures from Example 329.

m/z (ES+) [M+H]+=623.2

¹H NMR (500 MHz, DMSO) δ 11.30 (s, 1H), 10.93 (s, 1H), 9.13 (q, J=4.8 Hz, 1H), 8.40 (s, 1H), 8.16 (s, 1H), 8.04 (t, J=7.8 Hz, 1H), 7.92 (dd, J=7.7, 1.1 Hz, 1H), 7.77 (dd, J=8.0, 1.1 Hz, 1H), 7.57 (s, 1H), 7.39 (dd, J=8.0, 1.6 Hz, 1H), 7.20 (t, J=7.9 Hz, 1H), 7.05 (dd, J=7.7, 1.6 Hz, 1H), 5.56 (p, J=7.3 Hz, 1H), 5.49 (s, 1H), 4.87 (q, J=6.8 Hz, 1H), 4.07 (s, 1H), 3.89 (s, 2H), 3.86 (td, J=6.8, 1.9 Hz, 1H), 3.81-3.75 (m, 1H), 3.72 (t, J=7.0 Hz, 1H), 3.63 (t, J=7.1 Hz, 1H), 3.16 (s, 2H), 2.84 (d, J=4.8 Hz, 3H), 2.38 (s, 2H), 2.12-2.03 (m, 1H), 2.06 (s, 1H), 1.37 (d, J=6.7 Hz, 3H), 0.86-0.79 (m, 4H).

### Example 339

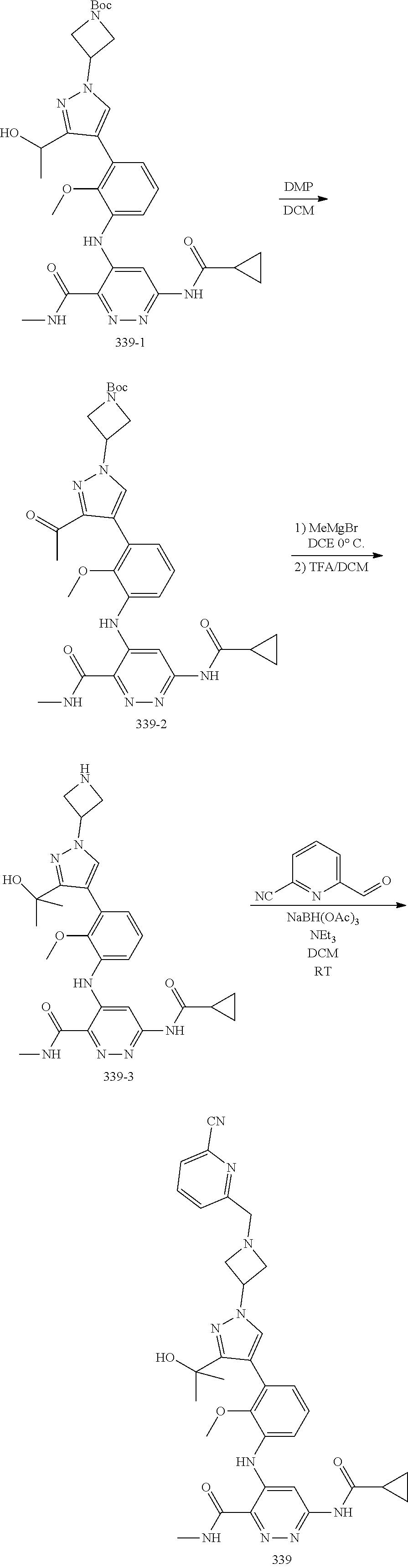 [SMILES: CNC(=O)c1nnc(NC(=O)C2CC2)cc1Nc1cccc(-c2cn(C3CN(C(=O)OC(C)(C)C)C3)nc2C(C)O)c1OC ; CNC(=O)c1nnc(NC(=O)C2CC2)cc1Nc1cccc(-c2cn(C3CN(C(=O)OC(C)(C)C)C3)nc2C(C)=O)c1OC ; CNC(=O)c1nnc(NC(=O)C2CC2)cc1Nc1cccc(-c2cn(C3CNC3)nc2C(C)(C)O)c1OC ; N#Cc1cccc(C=O)n1 ; CNC(=O)c1nnc(NC(=O)C2CC2)cc1Nc1cccc(-c2cn(C3CN(Cc4cccc(C#N)n4)C3)nc2C(C)(C)O)c1OC]

Step 1: Compound 339-1 was converted to compound 339-2 using standard oxidation conditions with Dess Martin periodinane.

Step 2: In a 40 mL vial was charged compound 339-2 (1.0 equiv) and magnesium bromide diethyl etherate (2.0 equiv) in 20 mL of DCE. The reaction was then cooled to 0° C. after which additional methylmagnesium bromide (3.5 equiv) was added and the reaction was stirred for 30 min until the reaction was determined to be complete by LC/MS analysis. The reaction was then quenched with aq NH₄Cl, then partitioned between MTBE and water. The organics were dried and concentrated. Crude material was then dissolved in 1:1 DCE/TFA and stirred at 40° C. for 1 h, after which it was concentrated and purified by flash silica gel chromatography (gradient: MeOH/DCM) to provide compound 339-3.

¹H NMR (500 MHZ, DMSO) δ 11.30 (s, 1H), 10.90 (s, 1H), 9.14 (q, J=4.8 Hz, 1H), 8.96 (s, 1H), 8.68 (s, 1H), 8.13 (s, 1H), 7.54 (s, 1H), 7.43 (dd, J=8.0, 1.6 Hz, 1H), 7.19 (t, J=7.9 Hz, 1H), 6.98 (dd, J=7.7, 1.6 Hz, 1H), 6.50 (s, OH), 6.09 (p, J=7.6 Hz, 1H), 5.52 (s, 1H), 4.43 (s, 2H), 4.36 (s, 2H), 3.40 (s, 3H), 2.83 (d, J=4.8 Hz, 3H), 2.12-2.03 (m, 1H), 1.31 (s, 6H), 1.23 (s, 1H), 0.87-0.77 (m, 4H).

Step 3: Compound 339-3 was converted to compound 339 using the analogous procedures from Example 329.

m/z (ES+) [M+H]+=637.2

### Example 341

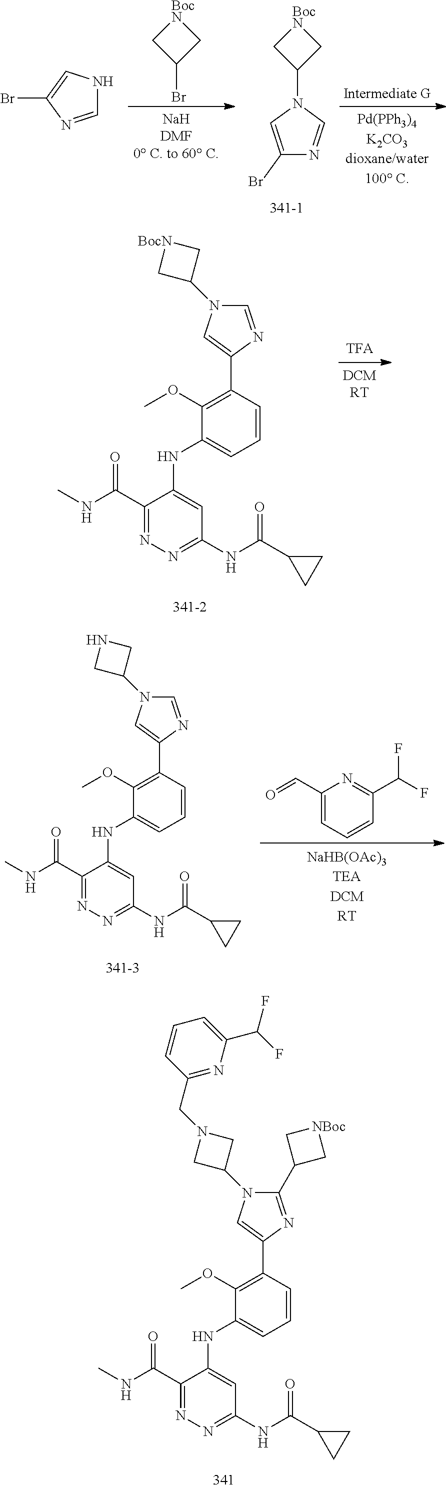

Step 1:4-Bromo-1H-imidazole (1 g, 1 equiv, 7 mmol) in DMF (35 mL) was slowly added NaH (1 g, 60% Wt, 5 equiv, 0.03 mol), the solution was stirred at 0° C. for 30 min, followed by the addition of tert-butyl 3-bromoazetidine-1-carboxylate (2 g, 1 equiv, 7 mmol) was added to the solution. The reaction was heated to 60° C. after which it was stirred for 1 h. The reaction mixture was then diluted with water (100 mL), and the aqueous phase was extracted with ethyl acetate (3×100 mL) three times. The combined organic layers were washed with saturated NaCl, dried over sodium sulfate, filtered, and concentrated in vacuo. The resulted solution was purified using C18 flash chromatography (gradeint: MeCN/water). Concentration in vacuo resulted in compound 341-1 (220 mg, 728 μmol, 10% yield) as a off-white solid.

m/z (ES+) [M+H]+=207.9

Step 2: Intermediate G (328 mg, 1.0 equiv, 702 μmol), compound 341-1 (212 mg, 1.0 equiv, 702 μmol), potassium carbonate (291 mg, 123 μL, 3.0 equiv, 2.10 mmol), 1,4-dioxane (5 mL), water (1 mL) were added to a 40 mL vial, and the reaction was purged with nitrogen. Tetrakis(triphenylphosphine) palladium (0) (162 mg, 0.2 equiv, 140 μmol) was added to the solution under nitrogen atmosphere after which the reaction mixture was stirred at 100° C. for 2 hours. The reaction mixture was then diluted with water (20 mL), and the aqueous phase was extracted with ethyl acetate (3×20 mL) three times. The combined organic layers were washed with saturated NaCl dried over sodium sulfate, filtered, and concentrated in vacuo. The resulting solution was purified using C18 flash chromatography (gradient: MeCN/water). Concentration in vacuo resulted in compound 341-2 (157 mg, 279 μmol, 40% yield) as a brown solid.

m/z (ES+) [M+H]+=563.2

Step 3: Compound 341-2 (110 mg, 1.0 equiv, 196 μmol), TFA (1 g, 1 mL, 7 equiv, 0.01 mol), DCM (3 mL) were added to a 40 mL vial. The mixture was stirred at 25° C. for 2 hours. The solution was neutralized by saturated NaHCO₃ solution to Ph=7, then extracted with DCM (3×50 mL) three times. The combined organic layers were washed with saturated NaCl, dried over sodium sulfate and filtered. Concentration in vacuo resulted in compound 341-3 (120 mg, 0.18 mmol, 93% yield) as a off-white solid.

m/z (ES+) [M+Na]+=485.4

Step 4: Compound 341-3 (120 mg, 1.0 equiv, 259 μmol), 6-(difluoromethyl) picolinaldehyde (48.9 mg, 1.2 equiv, 311 μmol), and TEA (105 mg, 145 μL, 4.0 equiv, 1.04 mmol) were stirred in DCM (4 mL) for 20 minutes at RT followed by the slow addition of sodium triacetoxyborohydride (110 mg, 2.0 equiv, 519 μmol) over 20 minutes followed by an additional 20 min of stirring. The reaction was concentrated, and the resulting crude material was purified by C-18 flash column chromatography (gradient: MeCN/water) to provide compound 341 (20 mg, 33 μmol, 13% yield) as a white amorphous solid.

m/z (ES+) [M+Na]+=604.4

¹H NMR (400 MHz, DMSO-d6) 11.30 (s, 1H), 10.95 (s, 1H), 9.17 (d, J=5.2 Hz, 1H), 8.12 (s, 1H), 8.04-7.94 (m, 2H), 7.93-7.86 (m, 2H), 7.60 (dd, J=11.7, 7.8 Hz, 2H), 7.33-7.26 (m, 1H), 7.22 (t, J=7.8 Hz, 1H), 6.93 (t, J=55.0 Hz, 1H), 5.02 (t, J=6.7 Hz, 1H), 3.93 (s, 2H), 3.82 (t, J=7.5 Hz, 2H), 3.67 (s, 3H), 3.51 (s, 2H), 2.87 (d, J=4.8 Hz, 3H), 2.08 (s, 1H), 0.85-0.78 (m, 4H).

### Example 342

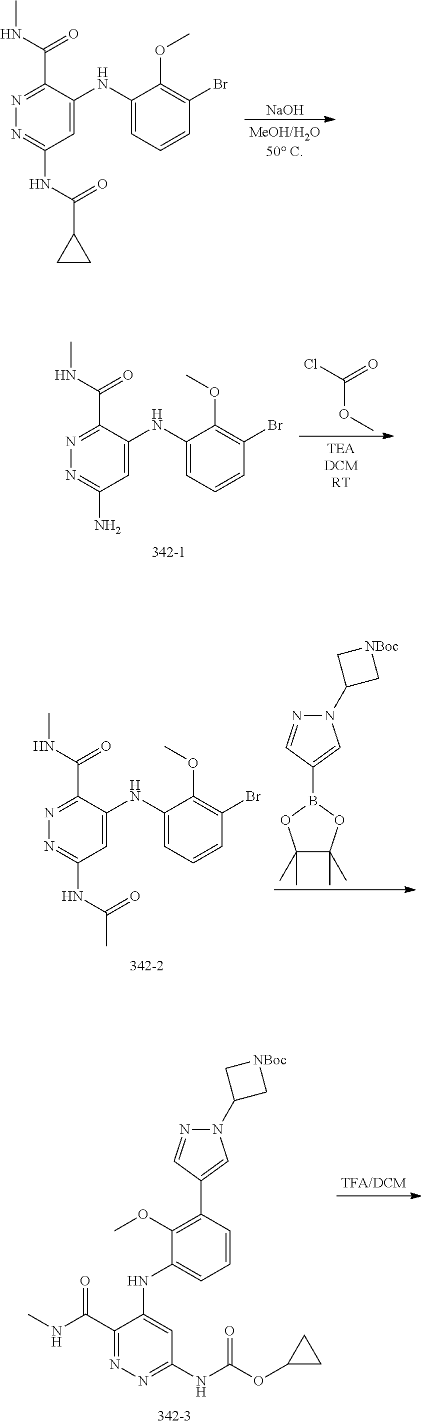

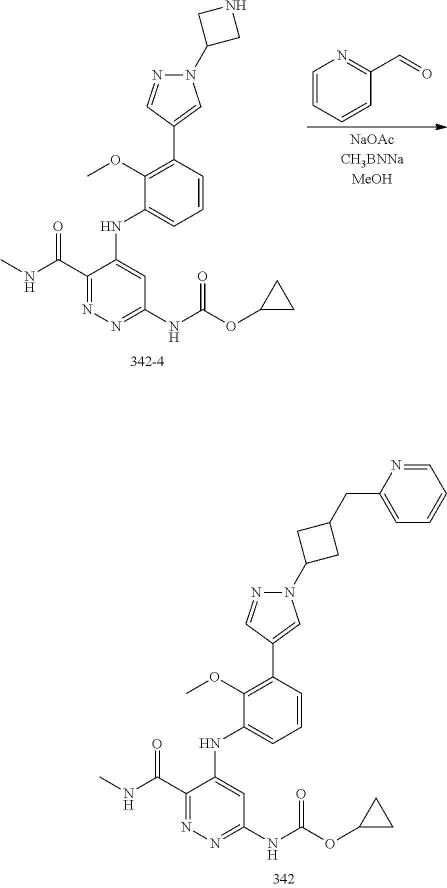 [SMILES: CNC(=O)c1nnc(NC(=O)OC2CC2)cc1Nc1cccc(-c2cnn(C3CNC3)c2)c1OC ; O=Cc1ccccn1 ; CNC(=O)c1nnc(NC(=O)OC2CC2)cc1Nc1cccc(-c2cnn(C3CC(Cc4ccccn4)C3)c2)c1OC]

Step 1: To a solution of Intermediate H (2 g, 1 equiv, 5 mmol) in MeOH (30 mL) was added a solution of NaOH (0.6 g, 3 equiv, 0.01 mol) in H₂O (15 mL), The mixture was stirred at 50° C. for 16 hour. The reaction mixture was diluted with H₂O (30 mL), and the aqueous phase was extracted with DCM (3×50 mL) three times. The combined organic layers were washed with brine, dried over sodium sulfate, filtered, and concentrated in vacuo. The resulting crude material was purified by C-18 Flash with (gradient: MeCN/Water) to provide compound 342-1 (1.4 g, 4.0 mmol, 80% yield) as a an orange-red solid.

m/z (ES+) [M+H]+=352.0

Step 2: A resealable reaction vial was charged with compound 342-1 (1.4 g, 1.0 equiv, 4.0 mmol), TEA (1.2 g, 3.0 equiv, 12 mmol), DCM (12 mL), and cyclopropyl carbonochloridate (0.57 g, 1.2 equiv, 4.8 mmol), and a stirbar before being evacuated and purged with nitrogen three times. The mixture was stirred at room temperature for 1 h. The reaction mixture was diluted with H₂O (30 mL), and the aqueous phase was extracted with DCM (3×50 mL) three times. The combined organic layers were washed with brine, dried over sodium sulfate, filtered, and concentrated in vacuo. The resulting crude material was purified by C-18 flash chromatography (gradient MeCN/water) to provide compound 342-2 (600 mg, 1.38 mmol, 35% yield) as a yellow solid.

m/z (ES+) [M+H]+=436.1

Step 3: A resealable reaction vial was charged with compound 342-2 (600 mg, 1.0 equiv, 1.38 mmol), tert-butyl 3-(4-(4,4,5,5-tetramethyl-1,3,2-dioxaborolan-2-yl)-1H-pyrazol-1-yl) azetidine-1-carboxylate (720 mg, 1.5 equiv, 2.06 mmol), Pd(dppf)Cl₂ (89.5 mg, 0.1 equiv, 138 μmol), CsF (627 mg, 3 equiv, 4.13 mmol), and a stirbar before being evacuated and purged with nitrogen three times. dioxane:H₂O (10 mL) was added, and the mixture was stirred at 80° C. for 2 h. The reaction mixture was diluted with H₂O (30 mL), and the aqueous phase was extracted with DCM (3×50 mL) three times. The combined organic layers were washed with brine, dried over sodium sulfate, filtered, and concentrated in vacuo. The resulting crude material was purified by HPLC (acetonitrile/water/0.1% formic acid). Lyophilization yielded compound 342-3 (500 mg, 864 μmol, 63% yield) as a yellow solid.

m/z (ES+) [M+H]+=579.3

Step 4: A resealable reaction vial was charged with compound 342-3 (490 mg, 1.0 equiv, 847 μmol), DCM:TFA (12 mL), and a stirbar before being evacuated and purged with nitrogen three times. The mixture was stirred at room temperature for 1 h. The mixture was concentrated under vacuum and diluted with H₂O (30 mL), and the aqueous phase was extracted with THF (3×50 mL) three times. The combined organic layers were washed with brine, dried over sodium sulfate, filtered, and concentrated in vacuo to get compound 342-4 (350 mg, 731 μmol, 86.4%) as a dark orange solid.

m/z (ES+) [M+H]+=479.3

Step 5: A round bottomed flask was charged with compound 342-4 (120 mg, 1.0 equiv, 251 μmol), picolinaldehyde (40.3 mg, 1.5 equiv, 376 μmol), sodium acetate (82.3 mg, 4.0 equiv, 1.00 mmol) and a stirbar. MeOH (2 mL) was added, and the solution was stirred at 0° C. for 20 min. Sodium (methylbutanylidene)amide (63.0 mg, 4.0 equiv, 1.00 mmol) was added, and the solution was stirred at 25° C. for 30 min until the reaction was determined to be complete by LC/MS analysis. The resulting crude material was purified by HPLC(Column: XBridge Prep OBD C18 Column, gradient: water (10 mM NH₄HCO₃)/MeCN Lyophilization yielded compound 342 (18.2 mg, 32.0 μmol, 13% yield) as a off-white amorphous solid.

¹H NMR (400 MHZ, DMSO-d6) 10.96 (s, 1H), 10.75 (s, 1H), 9.15 (q, J=4.8 Hz, 1H), 8.51 (d, J=5.0 Hz, 1H), 8.39 (s, 1H), 8.04 (s, 1H), 7.86 (s, 1H), 7.81-7.73 (m, 1H), 7.50 (d, J=7.8 Hz, 1H), 7.41 (d, J=7.8 Hz, 1H), 7.34 (d, J=7.9 Hz, 1H), 7.25 (p, J=7.8, 6.6 Hz, 2H), 5.13 (p, J=6.9 Hz, 1H), 4.05 (tt, J=6.3, 3.2 Hz, 1H), 3.83 (d, J=10.5 Hz, 4H), 3.61 (s, 5H), 2.87 (d, J=4.8 Hz, 3H), 0.74-0.63 (m, 4H).

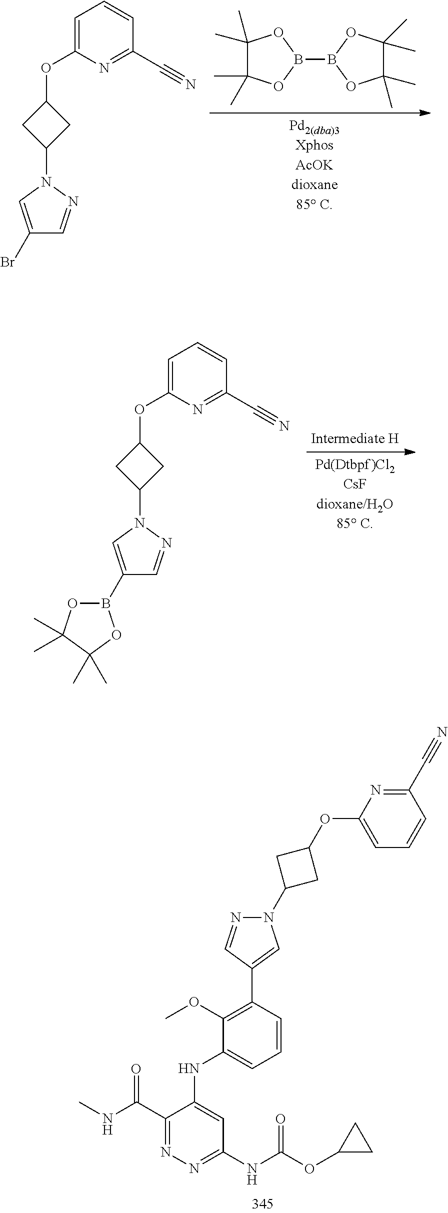

Step 1: A resealable reaction vial was charged with 6-(3-(4-bromo-1H-pyrazol-1-yl)cyclobutoxy) picolinonitrile (360 mg, 1.0 equiv, 1.13 mmol), 4,4,4′,4′,5,5,5′,5′-octamethyl-2,2′-bi(1,3,2-dioxaborolane) (430 mg, 1.5 equiv, 1.69 mmol), Pd₂(dba)₃ (103 mg, 0.1 equiv, 113 μmol), Xphos (53.8 mg, 0.1 equiv, 113 μmol), AcOK (0.33 g, 3 equiv, 3.38 mmol), and a stir bar before being evacuated and purged with nitrogen three times. 1,4-Dioxane (10 mL) was added, and the mixture was stirred at 85° C. for 2 hours. The reaction mixture was diluted with H₂O (30 mL), and the aqueous phase was extracted with DCM (3×50 mL) three times. The combined organic layers were washed with brine, dried over sodium sulfate, filtered, and concentrated in vacuo. The resulting crude material was purified by C-18 flash chromatography (gradient: acetonitrile/water) to get compound 345-1 (350 mg, 956 μmol, 85% yield) as an off-white solid.

m/z (ES+) [M+H]+=367.2

Step 2: A resealable reaction vial was charged compound 345-1 (119 mg, 1.0 equiv, 273 μmol), 6-(3-(4-(4,4,5,5-tetramethyl-1,3,2-dioxaborolan-2-yl)-1H-pyrazol-1-yl)cyclobutoxy) picolinonitrile (100 mg, 1.0 equiv, 273 μmol), CsF (124 mg, 3.0 equiv, 819 μmol), 1,4-Dioxane (4 mL), water (1 mL) were added to a 40 mL vial, the air in the reaction solution was replaced with nitrogen. 1,1′-Bis(di-t-butylphosphino) ferrocene palladium dichloride (35.6 mg, 0.2 equiv, 54.6 μmol) was added to the solution under nitrogen atmosphere. The reaction mixture was stirred at 85° C. for 2 hours. The reaction mixture was diluted with H₂O (30 mL), and the aqueous phase was extracted with DCM (3×50 mL) three times. The combined organic layers were washed with brine, dried over sodium sulfate, filtered, and concentrated in vacuo. The resulting crude material was purified by C-18 flash chromatography (gradient: MeCN/Water). Lyophilization yielded compound 345 (29.9 mg, 50.2 μmol, 18% yield) as an off-white amorphous solid as a mixture of cis and trans diastereomers.

m/z (ES+) [M+H]+=596.2

¹H NMR (400 MHZ, DMSO-d6) 10.95 (s, 1H), 10.74 (s, 1H), 9.14 (d, J=4.9 Hz, 1H), 8.33 (s, 1H), 8.03 (s, 1H), 8.01-7.93 (m, 1H), 7.85 (s, 1H), 7.69 (d, J=7.1 Hz, 1H), 7.50 (d, J=7.5 Hz, 1H), 7.33 (d, J=7.8 Hz, 1H), 7.29-7.19 (m, 2H), 5.11 (t, J=7.2 Hz, 1H), 4.78 (t, J=8.1 Hz, 1H), 4.05 (s, 1H), 3.60 (s, 3H), 3.07 (s, 2H), 2.87 (d, J=4.7 Hz, 3H), 2.71 (s, 2H), 0.72-0.64 (m, 3H).

### Example 346

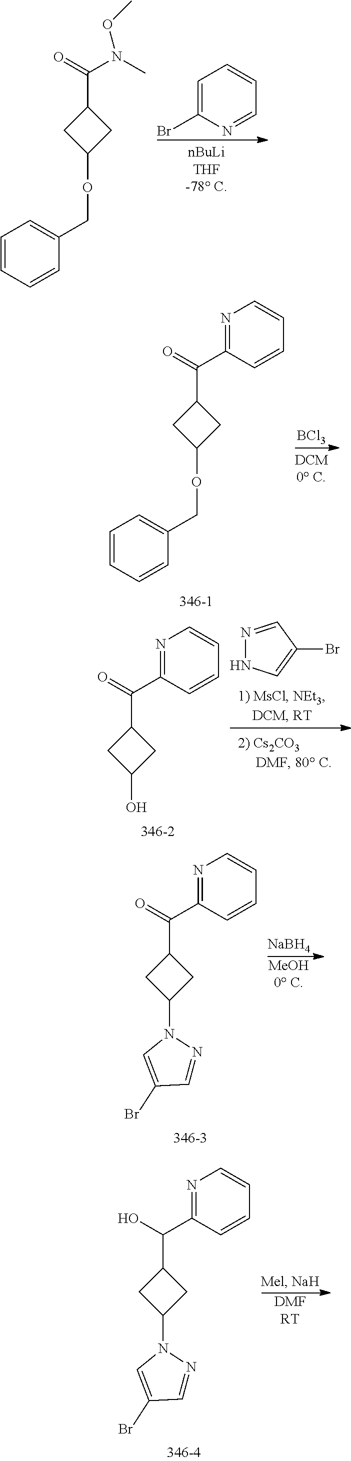

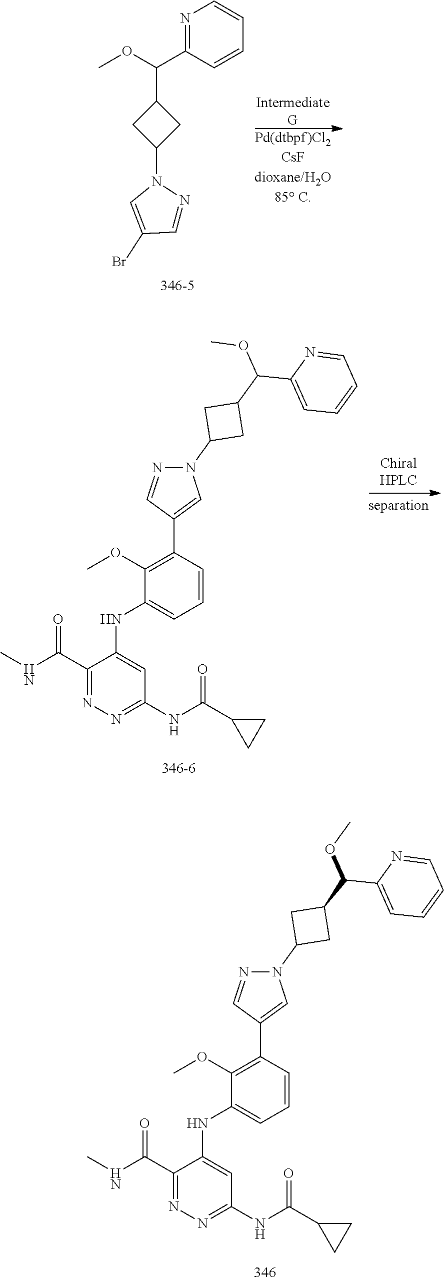

Step 1: Under nitrogen atmosphere, to a solution of 2-bromopyridine (1.3 g, 1.0 equiv, 8.2 mmol) in THF (20 mL) was stirred for 5 min at −78° C., and added dropwise butyllithium (1.1 g, 6.6 mL, 2.5 molar, 2.0 equiv, 16 mmol) was stirred for 1 h at −78° C., after which 3-(benzyloxy)-N-methoxy-N-methylcyclobutane-1-carboxamide (2.7 g, 1.3 equiv, 11 mmol) was added. The mixture was stirred for 2 h at −78° C. The reaction was then washed with saturated NH₄Cl, extracted with ethyl acetate (150 mL×3), and dried with Na₂SO₄ for 5 min, then filtered and concentrated in vacuo. The mixture was purified by reverse phase column chromatography (gradient: MeCN/H₂O), and the product containing fractions were concentrated yielding to the desired product compound 346-1 (1 g, 4 mmol, 50% yield).

m/z (ES+) [M+H]+=268.0

Step 2: To a solution of compound 346-1 (1 g, 1 equiv, 4 mmol) in DCM (15 mL) was stirred at 0° C. and under N₂ atmosphere, was added trichloroborane (1 g, 0.01 L, 1 molar, 3.0 equiv, 0.01 mol) at 0° C., then stirred for 2 h at 25° C., under N₂ atmosphere. The reaction was then washed with saturated Na₂CO₃, extracted with DCM (50 mL×3), and dried with Na₂SO₄ for 5 min, then filtered and concentrated in vacuo. The mixture was purified by reverse phase column chromatography (gradeint: MeCN/H₂O), the product containing fractions were concentrated to provide compound 346-2 (540 mg, 3.05 mmol, 80% yield).

m/z (ES+) [M+H]+=178.1

Step 3: A mixture of compound 346-2 (600 mg, 1.0 equiv, 3.39 mmol), triethylamine (1.37 g, 4.0 equiv, 13.5 mmol) were stirred in DCM (10 mL) at RT after which methanesulfonyl chloride was added (582 mg, 1.5 equiv, 5.08 mmol), and the reaction was stirred for 5 h at rt. After the reaction was determined to be complete, H₂O (20 mL) was added, and the moisture was extracted with DCM (30 mL×3), and dried with Na₂SO₄ for 5 min, then filtered and concentrated in vacuo.

The crude material was redissolved in DMF (10 mL), after which was added the 4-bromo-1H-pyrazole (547 mg, 1.1 equiv, 3.72 mmol) and cesium carbonate (3.0 equiv), and the reaction was stirred for overnight at 80° C. After the reaction was complete, H₂O (100 mL) was added, and the mixture was extracted with ethyl acetate (130 ml×3), and dried with Na₂SO₄ for 5 min, then filtered and concentrated in vacuo. The mixture was purified by reverse phase chromatography (gradient: MeCN/H₂O), and the product containing fractions were concentrated yielding to the desired product compound 346-3 as a yellow oil (900 mg, 2.94 mmol, 87% yield).

m/z (ES+) [M+H]+=305.8

Step 4: A solution of compound 346-3 (890 mg, 1.0 equiv, 2.91 mmol) were stirred in MeOH (10 mL) at 0° C., then slowly added NaBH₄ (550 mg, 5.0 equiv 14.5 mmol), stirred for 3 hour at rt. After the reaction, was added H₂O (20 mL), then filtered and concentrated in vacuo. Water was added (100 mL), extracted with ethyl acetate (130 mL×3), and dried with Na₂SO₄ for 5 min, then filtered and concentrated in vacuo. The mixture was purified by reverse phase column chromatography (gradient: MeCN/H₂O), the product containing fractions were concentrated yielding to the desired product compound 346-4 as a yellow oil (500 mg, 1.62 mmol, 56%).

m/z (ES+) [M+H]+=308.2

Step 5: A solution of compound 346-4 (570 mg, 1.0 equiv, 1.85 mmol) was stirred in DMF (10 mL) at 0° C., after which sodium hydride (0.13 g, 3 equiv, 5.55 mmol) was added, stirred for 1 h at rt. Iodomethane (289 mg, 1.1 equiv, 2.03 mmol) was added and stirred at 25° C. for overnight. Water was added (100 ml), and the moisture was extracted with ethyl acetate (130 mL×3), and dried with Na₂SO₄ for 5 min, then filtered and concentrated in vacuo. The mixture was purified by reverse phase chromatography (gradient: MeCN/H₂O) and the product containing fractions were concentrated yielding to the desired product as a yellow oil, product 346-5 (570 mg, 1.77 mmol, 96%) m/z (ES+) [M+H]+=322.2

Step 6: A mixture of Intermediate G (530 mg, 1.0 equiv, 1.13 mmol), compound 346-5 (402 mg, 1.1 equiv, 1.25 mmol), cesium fluoride (345 mg, 2.0 equiv, 2.27 mmol), Pd(dtbpf)Cl₂ (148 mg, 0.2 equiv, 227 μmol) was stirred in dioxane/H₂O (15 mL) (4/1 v:v) at 85° C. for 5 hour under N₂ atmosphere. Brine was then added (100 mL), and the mixture was extracted with DCM (130 mL×3), and dried with Na₂SO₄ for 5 min, then filtered and concentrated in vacuo. The mixture was purified by reverse phase column chromatography (gradient: MeCN/H₂O), the product containing fractions were concentrated yielding to the desired product compound 346-6 as a yellow oil (660 mg, 1.13 mmol, 100% yield).

m/z (ES+) [M+H]+=583.2

Compound 346 was isolated from consecutive chiral HPLC separations as a single enantiomer of the trans cyclobutyl diasteromers.

Purification 1: CHIRALPAK IE, 2*25 cm, 5 um; Mobile Phase A: MtBE (0.3% IPA)-HPLC, Mobile Phase B: EtOH:DCM=1:1-HPLC; Flow rate: 20 mL/min; Gradient: 40% B to 40% B in 11 min; Wave Length: 220/254 nm; RT1 (min): 7.224; RT2 (min): 9.184; Sample

Solvent: EtOH:DCM=1:1-HPLC; Injection Volume: 0.5 mL; Number Of Runs: 13

Purification 2: Column: Lux 3 um Cellulose-2, 4.6*50 mm, 3 um; Mobile Phase A: Hex (0.2% DEA): EtOH=50:50; Flow rate: 1 mL/min; Gradient: 0% B to 0% B; Injection Volume: 5 μl mL

Purification 3: Lux 5 um Cellulose-2, 2.12*25 cm, 5 um; Mobile Phase A: Hex (0.3% IP Amine)-HPLC, Mobile Phase B: EtOH-HPLC; Flow rate: 20 mL/min; Gradient: 50% B to 50% B in 30 min; Wave Length: 220/254 nm; RT1 (min): 18.37; RT2 (min): 21.22; Sample Solvent: EtOH-HPLC; Injection Volume: 0.9 mL; Number Of Runs: 12

m/z (ES+) [M+H]+=583.5

¹H NMR (400 MHZ, DMSO-d6) 11.31 (s, 1H), 10.98 (s, 1H), 9.17 (q, J=4.7 Hz, 1H), 8.55 (ddd, J=4.9, 1.8, 0.9 Hz, 1H), 8.25-8.21 (m, 1H), 8.16 (s, 1H), 7.97 (d, J=0.7 Hz, 1H), 7.82 (td, J=7.7, 1.8 Hz, 1H), 7.47 (dd, J=7.8, 1.6 Hz, 1H), 7.39 (dt, J=7.9, 1.2 Hz, 1H), 7.35-7.26 (m, 2H), 7.19 (t, J=7.9 Hz, 1H), 4.73 (t, J=7.8 Hz, 1H), 4.30 (d, J=5.3 Hz, 1H), 3.59 (s, 3H), 3.23 (s, 3H), 2.87 (d, J=4.8 Hz, 3H), 2.50-2.38 (m, 4H), 2.22 (ddd, J=11.1, 7.5, 4.7 Hz, 1H), 2.08 (h, J=5.8 Hz, 1H), 0.86-0.79 (m, 4H).

TABLE 25
The following diastereomers were isolated under analogous conditions.

| | | LCMS m/z | |
|---|---|---|---|
| Compound | Structure | (ES+) | ¹H NMR |
| 350* | 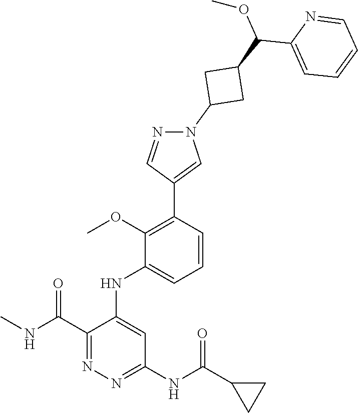 | 383.5 [M + H]+ | ¹H NMR (400 MHz, DMSO-d6) 11.31 (s, 1H), 10.96 (s, 1H), 9.16 (d, J = 4.9 Hz, 1H), 8.57 (ddd, J = 4.9, 1.8, 0.9 Hz, 1H), 8.27 (d, J = 0.7 Hz, 1H), 8.15 (s, 1H), 7.97 (s, 1H), 7.84 (td, J = 7.7, 1.8 Hz, 1H), 7.48-7.40 (m, 2H), 7.37- 7.25 (m, 2H), 7.19 (t, J = 7.9 Hz, 1H), 5.01 (p, J = 7.7 Hz, 1H), 4.39 (d, J = 7.2 Hz, 1H), 3.58 (s, 3H), 3.27 (s, 3H), 2.87 (d, J = 4.8 Hz, 3H), 2.71 (dd, J = 11.1, 6.3 Hz, 1H), 2.55 (dd, J = 8.3, 3.4 Hz, 2H), 2.37 (dt, J = 7.7, 3.5 Hz, 1H), 2.08 (dq, J = 8.0, 5.3, 3.6 Hz, 1H), 0.82 (d, J = 8.0 Hz, 4H). |
| 352* | 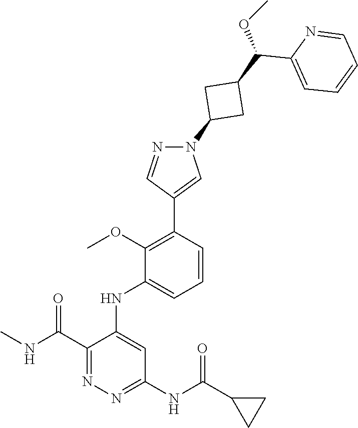 | 383.5 [M + H]+ | ¹H NMR (400 MHz, DMSO-d6) 11.31 (s, 1H), 10.98 (s, 1H), 9.17 (q, J = 4.7 Hz, 1H), 8.55 (ddd, J = 4.9, 1.8, 0.9 Hz, 1H), 8.25- 8.21 (m, 1H), 8.16 (s, 1H), 7.97 (d, J = 0.7 Hz, 1H), 7.82 (td, J = 7.7, 1.8 Hz, 1H), 7.47 (dd, J = 7.8, 1.6 Hz, 1H), 7.39 (dt, J = 7.9, 1.2 Hz, 1H), 7.35-7.26 (m, 2H), 7.19 (t, J = 7.9 Hz, 1H), 4.73 (t, J = 7.8 Hz, 1H), 4.30 (d, J = 5.3 Hz, 1H), 3.59 (s, 3H), 3.23 (s, 3H), 2.87 (d, J = 4.8 Hz, 3H), 2.50-2.38 (m, 4H), 2.22 (ddd, J = 11.1, 7.5, 4.7 Hz, 1H), 2.08 (h, J = 5.8 Hz, 1H), 0.86- 0.79 (m, 4H). |
| 353* | 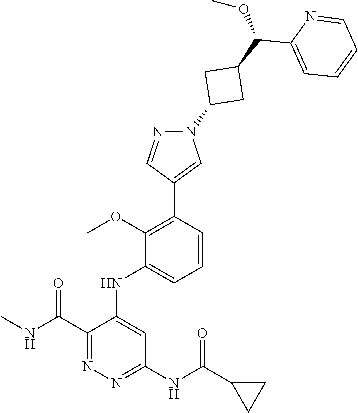 | 383.5 [M + H]+ | ¹H NMR (400 MHz, DMSO-d6) 11.31 (s, 1H), 10.96 (s, 1H), 9.16 (d, J = 4.9 Hz, 1H), 8.57 (ddd, J = 4.9, 1.8, 0.9 Hz, 1H), 8.27 (d, J = 0.7 Hz, 1H), 8.15 (s, 1H), 7.97 (s, 1H), 7.84 (td, J = 7.7, 1.8 Hz, 1H), 7.48-7.40 (m, 2H), 7.37- 7.25 (m, 2H), 7.19 (t, J = 7.9 Hz, 1H), 5.01 (p, J = 7.7 Hz, 1H), 4.39 (d, J = 7.2 Hz, 1H), 3.58 (s, 3H), 3.27 (s, 3H), 2.87 (d, J = 4.8 Hz, 3H), 2.71 (dd, J = 11.1, 6.3 Hz, 1H), 2.55 (dd, J = 8.3, 3.4 Hz, 2H), 2.37 (dt, J = 7.7, 3.5 Hz, 1H), 2.08 (dq, J = 8.0, 5.3, 3.6 Hz, 1H), 0.82 (d, J = 0.82 Hz, 4H). |

*Absolute stereochemistry arbitrarily assigned.

### Example 348 and Example 349

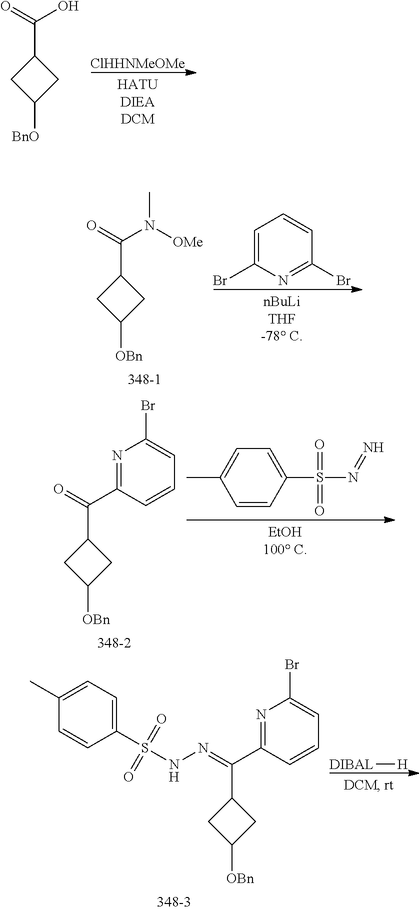

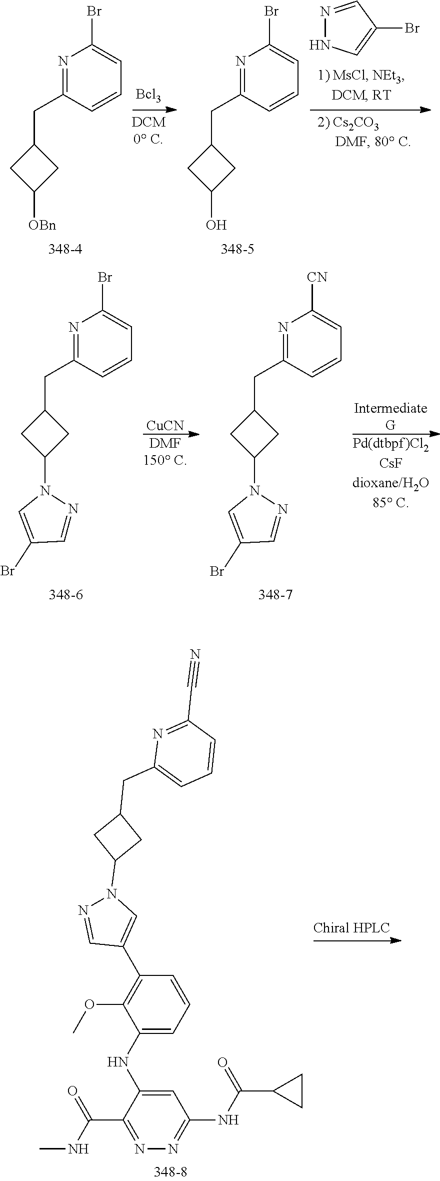

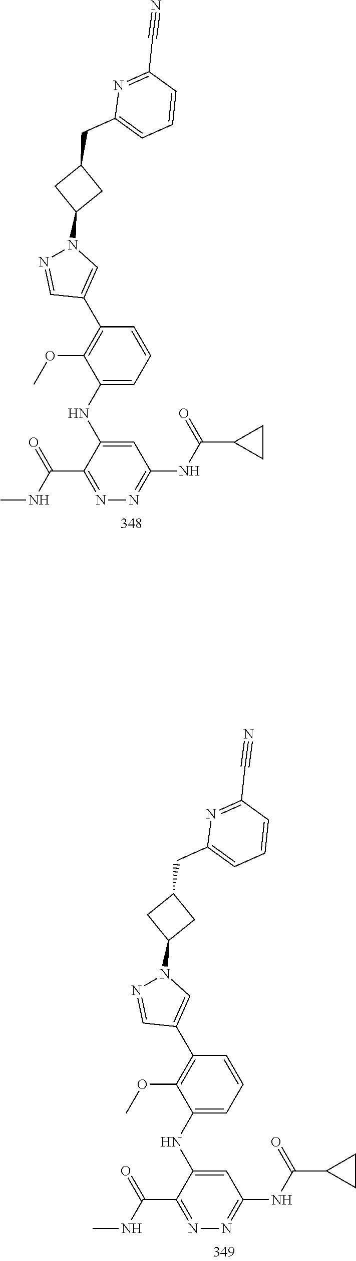 [SMILES: CNC(=O)c1nnc(NC(=O)C2CC2)cc1Nc1cccc(-c2cnn([C@H]3C[C@@H](Cc4cccc(C#N)n4)C3)c2)c1OC ; CNC(=O)c1nnc(NC(=O)C2CC2)cc1Nc1cccc(-c2cnn([C@H]3C[C@H](Cc4cccc(C#N)n4)C3)c2)c1OC]

Step 1: To a stirred solution of 3-(benzyloxy)cyclobutane-1-carboxylic acid (2.2 g, 1.0 equiv, 11 mmol), N,O-dimethylhydroxylamine hydrochloride (1.2 g, 1.2 equiv, 13 mmol) and HATU (8.1 g, 2.0 equiv, 21 mmol) in DCM (10 mL) was stirred at room temperature for 0.5 h, after which disopropylethylamine (4.1 g, 5.5 mL, 3.0 equiv, 32 mmol) is added at room temperature. The resulting mixture was stirred at room temperature for 2 h. The reaction was quenched with water and extracted with DCM. The organic layer was washed with brine, dried over Na₂SO₄ and evaporated. The resulted solution was purified using C18 flash column chromatography (gradient: MeCN/water) to provide compound 348-1 (2.1 g, 8.4 mmol, 79%) as a light-yellow solid.

m/z (ES+) [M+H]+=250.1

Step 2: Under nitrogen atmosphere, to a solution of 2,6-dibromopyridine (2.3 g, 1.0 equiv, 9.6 mmol) in THF (10 mL) was stirred for 5 min at −78° C., followed by dropwise addition of n-butyllithium (0.62 g, 3.9 mL, 2.5 molar, 1.0 equiv, 9.6 mmol). The reaction was stirred for 1 h at −78° C., after which compound 348-1 (2.4 g, 1.0 equiv, 9.6 mmol) was added. The mixture was stirred for 2 h at −78° C. The reaction was then washed with saturated NH₄Cl, extracted with ethyl acetate (150 mL×3), and dried with Na₂SO₄ for 5 min, then filtered and concentrated in vacuo.

The mixture was purified by reverse phase column chromatography (gradient: MeCN/water). The product containing fractions were concentrated yielding to the desired product compound 348-2 as a yellow oil (2.1 g, 6.1 mmol, 63% yield).

m/z (ES+) [M+H]+=346.2

Step 3: To a solution of compound 348-2 (2.1 g, 1.0 equiv, 6.1 mmol), tosyldiazene (1.3 g, 1.2 equiv, 7.3 mmol) in EtOH (15 mL) was stirred at 100° C. for 2 h. The solution was concentrated to dryness. The reaction was quenched with water and extracted with DCM. The organic layer was washed with brine, dried over Na₂SO₄ and evaporated. The resulted solution was purified using C18 flash chromatography with the following conditions (gradient: MeCN/water). This provided compound 348-3 (1.6 g, 3.1 mmol, 51% yield) as a yellow oil.

m/z (ES+) [M+H]+=513.9

Step 4: To a stirred solution of compound 348-3 (1.6 g, 1.0 equiv, 3.1 mmol) in DCM (15 mL), and followed by the addition of diisobutylaluminum hydride (4.4 g, 5.4 mL, 10 equiv, 31 mmol) at room temperature. The resulting mixture was stirred at room temperature for 1 h followed by the slow addition of NaOH (2M) at rt until pH 10 was reached. The mixture was then extracted with DCM, dried over Na₂SO₄ and evaporated. The resulted solution was purified using C18 flash chromatography (gradient: MeCN/water) to provide compound 348-4 (710 mg, 2.14 mmol, 69% yield) as a light yellow solid.

m/z (ES+) [M+H]+=332.2

Step 5: Under nitrogen atmosphere, to a stirred solution of compound 348-4 (710 mg, 1.0 equiv, 2.14 mmol) in DCM (8 mL) was stirred at 0° C. for 5 min, followed by the addition of trichloroborane (1.25 g, 5.0 equiv, 10.7 mmol) at 0° C., and stirred at 25° C. for 1 h. The reaction was washed with sat. NaHCO₃ and extracted with DCM, dried over Na₂SO₄ and evaporated. The resulted solution was purified using C18 flash chromatography (gradient: MeCN/water) to provide compound 348-5 (440 mg, 1.82 mmol, 85% yield) as a light yellow solid.

m/z (ES+) [M+H]+=242.2

Step 6: To a stirred solution compound 348-5 (440 mg, 1.0 equiv, 1.82 mmol), triethylamine (552 mg, 3.0 equiv, 5.45 mmol) in DCM (5 mL) at 0° C. for 5 min followed by the addition of methanesulfonyl chloride (250 mg, 1.2 equiv, 2.18 mmol) at 0° C. The resulting mixture was stirred at room temperature for 1 h. The reaction was washed with brine and extracted with DCM, dried over Na₂SO₄ and evaporated. To the mixture was added 4-bromo-1H-pyrazole (294 mg, 1.1 equiv, 2.00 mmol), Cs₂CO₃ (1.78 g, 3.0 equiv, 5.45 mmol) in DMF (6 mL) at 80° C. for 1 hour. The reaction was washed with brine and extracted with DCM, dried over Na₂SO₄ and evaporated. The resulted solution was purified using C18 flash chromatography (gradient: MeCN/water) to provide compound 348-6 (486 mg, 1.31 mmol, 72% yield) as a light yellow solid.

m/z (ES+) [M+H]+=371.8

Step 7: A stirred solution of compound 348-6 (486 mg, 1.0 equiv, 1.31 mmol), cyanocopper (117 mg, 1.0 equiv, 1.31 mmol) in DMF (5 mL) was heated to at 150° C. for 4 hour. The reaction was washed with brine and extracted with DCM, dried over Na₂SO₄ and evaporated. The resulted solution was purified using C18 flash chromatography (gradient: MeCN/water) to provided 348-7 (220 mg, 694 μmol, 53% yield) as a light yellow solid.

m/z (ES+) [M+H]+=317.2

Step 8: To a stirred solution of Intermediate G (220 mg, 1.0 equiv, 471 μmol) was added compound 348-7 (164 mg, 1.1 equiv, 518 μmol), 1,1′-Bis(di-t-butylphosphino) ferrocene palladium dichloride (46.0 mg, 0.15 equiv, 70.6 μmol), cesium fluoride (215 mg, 3.0 equiv, 1.41 mmol) in 1,4-dioxane (8 mL) and H₂O (2 mL) and the resulting solution was stirred at 85° C. for 3 hour under nitrogen atmosphere. The reaction was washed with brine and extracted with DCM, dried over Na₂SO₄ and evaporated. The column was purified with HPLC(Column: XSelect CSH Fluoro Phenyl, gradient: water (0.1% FA)/MeCN) to provide compound 348-8 (120 mg, 208 μmol, 44 yield) as a off-white solid.

m/z (ES+) [M+H]+=578.2

The cis and trans isomers were separated by Chiral HPLC:

Conditions: CHIRALPAK IF, 2*25 cm, 5 um; Mobile Phase A: MtBE (0.3% IPA)-HPLC, Mobile Phase B: ACN; Flow rate: 20 mL/min; Gradient: 10% B to 10% B in 27 min; Wave Length: 220/254 nm; RT1 (min): 17.35; RT2 (min): 20.89; Sample Solvent: ACN; Injection Volume: 0.7 mL; Number Of Runs: 3).

Compound 348 (cis diastereomer):

m/z (ES+) [M+H]+=578.4

¹H NMR (400 MHZ, DMSO-d6) 11.31 (s, 1H), 10.98 (s, 1H), 9.16 (t, J=4.8 Hz, 1H), 8.28 (d, J=0.8 Hz, 1H), 8.16 (s, 1H), 8.03-7.94 (m, 2H), 7.88 (dd, J=7.7, 1.1 Hz, 1H), 7.66 (dd, J=7.9, 1.1 Hz, 1H), 7.47 (dd, J=7.8, 1.6 Hz, 1H), 7.29 (dd, J=7.9, 1.6 Hz, 1H), 7.20 (t, J=7.9 Hz, 1H), 4.78 (p, J=8.3 Hz, 1H), 3.59 (s, 3H), 3.04 (d, J=6.5 Hz, 2H), 2.87 (d, J=4.9 Hz, 3H), 2.55 (d, J=7.7 Hz, 2H), 2.30 (t, J=8.9 Hz, 2H), 2.07 (dt, J=7.0, 5.4 Hz, 1H), 1.24 (s, 1H), 0.88-0.79 (m, 4H).

### Compound 349 (Trans Diastereomer):

m/z (ES+) [M+H]+=578.35; HPLC tR=0.837 min.

¹H NMR (400 MHZ, DMSO-d6) 11.31 (s, 1H), 10.97 (s, 1H), 9.17 (q, J=4.7 Hz, 1H), 8.28 (s, 1H), 8.15 (s, 1H), 8.04-7.96 (m, 2H), 7.89 (dd, J=7.7, 1.1 Hz, 1H), 7.69 (dd, J=8.0, 1.1 Hz, 1H), 7.46 (dd, J=7.8, 1.5 Hz, 1H), 7.29 (dd, J=7.9, 1.5 Hz, 1H), 7.19 (t, J=7.9 Hz, 1H), 5.11 (p, J=7.7 Hz, 1H), 3.59 (s, 3H), 3.10 (d, J=7.9 Hz, 2H), 2.87 (d, J=4.7 Hz, 3H), 2.80 (dq, J=8.8, 4.2 Hz, 1H), 2.63 (tdd, J=9.2, 7.3, 2.4 Hz, 2H), 2.31 (td, J=9.0, 8.5, 3.9 Hz, 2H), 2.14-2.03 (m, 1H), 0.88-0.79 (m, 4H).

### Example 361

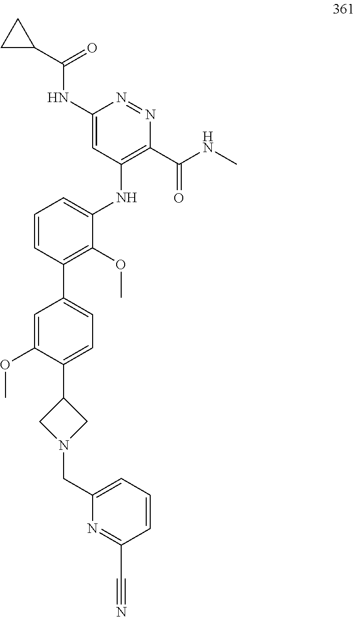 [SMILES: CNC(=O)c1nnc(NC(=O)C2CC2)cc1Nc1cccc(-c2ccc(C3CN(Cc4cccc(C#N)n4)C3)c(OC)c2)c1OC]

Compound 362 was prepared using analogous procedures as those in Example 182 except that tert-butyl 3-(4-bromo-2-methoxyphenyl) azetidine-1-carboxylate was used in place of 2-(azetidin-3-yl)-5-bromopyridine and the Boc group was deprotected under standard TFA/DCM deprotection conditions.

LC/MS (ESI+): m/z=619.2 [M+H]+

TABLE 26
The following compounds were prepared using analogous procedures as those used for the
formation of Example 362.

| | | LCMS m/z | |
|---|---|---|---|
| Compound | Structure | (ES+) | ¹H NMR |
| 359 | 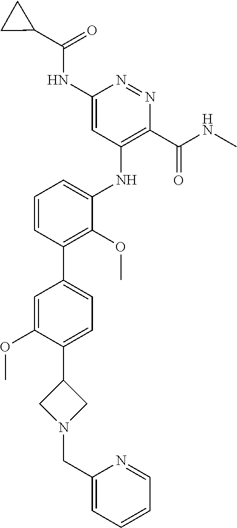 [SMILES: CNC(=O)c1nnc(NC(=O)C2CC2)cc1Nc1cccc(-c2ccc(C3CN(Cc4ccccn4)C3)c(OC)c2)c1OC] | 594.2 [M + H]+ | ¹H NMR (400 MHz, DMSO) δ 11.32 (s, 1H), 10.98 (s, 1H), 9.16 (d, J = 5.0 Hz, 1H), 8.65-8.49 (m, 1H), 8.18 (s, 1H), 7.80 (td, J = 7.7, 1.8 Hz, 1H), 7.47-7.41 (m, 2H), 7.36 (d, J = 7.8 Hz, 1H), 7.33-7.20 (m, 3H), 7.16 (d, J = 7.2 Hz, 2H), 4.10-3.87 (m, 4H), 3.81 (s, 3H), 3.57-3.43 (m, 3H), 3.38 (s, 3H), 2.85 (d, J = 4.8 Hz, 3H), 2.08 (q, J = 7.0, 6.1 Hz, 1H), 0.94-0.67 (m, 4H). |
| 360 | 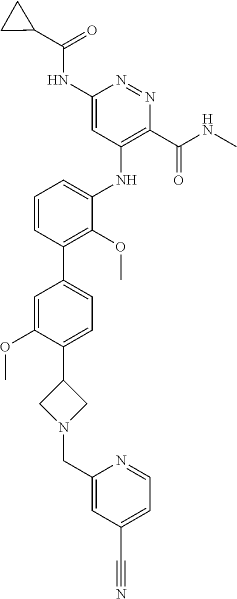 [SMILES: CNC(=O)c1nnc(NC(=O)C2CC2)cc1Nc1cccc(-c2ccc(C3CN(Cc4cc(C#N)ccn4)C3)c(OC)c2)c1OC] | 619.2 [M + H]+ | ¹H NMR (500 MHz, DMSO) δ 11.31 (s, 1H), 10.97 (s, 1H), 9.15 (q, J = 4.8 Hz, 1H), 8.79 (dd, J = 5.0, 0.9 Hz, 1H), 8.18 (s, 1H), 7.85 (s, 1H), 7.79 (dd, J = 5.0, 1.6 Hz, 1H), 7.44 (dd, J = 7.8, 1.8 Hz, 1H), 7.36 (d, J = 7.7 Hz, 1H), 7.27 (t, J = 7.8 Hz, 1H), 7.22 (dd, J = 7.7, 1.8 Hz, 1H), 7.16 (d, J = 7.5 Hz, 2H), 4.16- 3.90 (m, 6H), 3.81 (s, 3H), 3.52- 3.25 (m, 4H), 2.86 (d, J = 4.8 Hz, 3H), 2.09 (tt, J = 7.5, 5.0 Hz, 1H), 0.99-0.62 (m, 4H). |

### Example 363

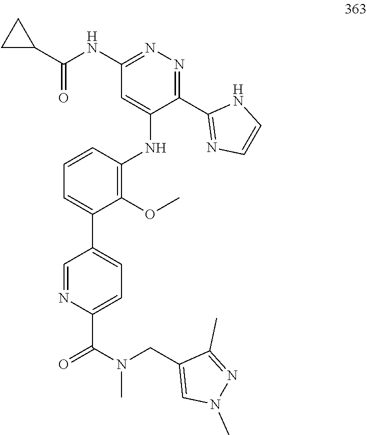 [SMILES: COc1c(Nc2cc(NC(=O)C3CC3)nnc2-c2ncc[nH]2)cccc1-c1ccc(C(=O)N(C)Cc2cn(C)nc2C)nc1]

Compound 363 was prepared using analogous procedures as those in Example 366 except that 1-(1,3-dimethyl-1H-pyrazol-4-yl)-N-methylmethanamine was used in place of 2-((methylamino)methyl) isonicotinonitrile.

m/z (ES⁺) [M+H]⁺=593.4

¹H NMR (400 MHZ, DMSO-d6) 13.30 (s, 1H), 11.59 (d, J=2.3 Hz, 1H), 11.22 (s, 1H), 8.78 (d, J=2.2 Hz, 1H), 8.26 (d, J=1.5 Hz, 1H), 8.16 (ddd, J=8.0, 5.3, 2.2 Hz, 1H), 7.69 (dd, J=12.9, 8.0 Hz, 1H), 7.60 (dd, J=7.6, 3.0 Hz, 2H), 7.40-7.28 (m, 3H), 7.25 (s, 1H), 4.48 (d, J=27.0 Hz, 2H), 3.73 (d, J=13.9 Hz, 3H), 3.43 (s, 3H), 3.04-2.87 (m, 3H), 2.18-1.93 (m, 4H), 0.83 (d, J=6.2 Hz, 4H).

### Example 364

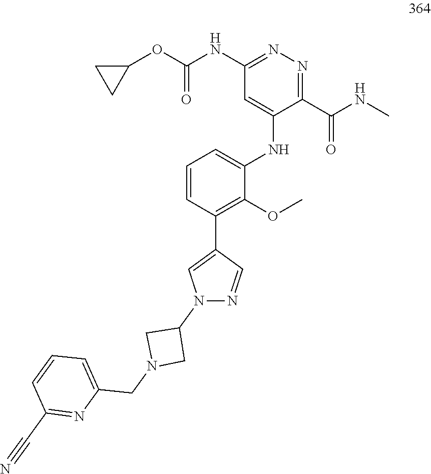 [SMILES: CNC(=O)c1nnc(NC(=O)OC2CC2)cc1Nc1cccc(-c2cnn(C3CN(Cc4cccc(C#N)n4)C3)c2)c1OC]

Compound 364 was prepared using analogous conditions as those in Example 342.

m/z (ES⁺) [M+H]⁺=595.4

¹H NMR (400 MHZ, DMSO-d6) 10.96 (s, 1H), 10.75 (s, 1H), 9.15 (q, J=4.8 Hz, 1H), 8.41 (s, 1H), 8.10-8.01 (m, 2H), 7.94 (dd, J=7.8, 1.1 Hz, 1H), 7.86 (s, 1H), 7.77 (dd, J=8.0, 1.1 Hz, 1H), 7.51 (dd, J=7.8, 1.6 Hz, 1H), 7.34 (dd, J=8.0, 1.6 Hz, 1H), 7.24 (t, J=7.9 Hz, 1H), 5.14 (p, J=6.9 Hz, 1H), 4.05 (tt, J=6.3, 3.3 Hz, 1H), 3.91 (s, 2H), 3.84 (td, J=7.1, 1.6 Hz, 2H), 3.68-3.57 (m, 5H), 2.87 (d, J=4.8 Hz, 3H), 0.74-0.61 (m, 4H).

### Example 366

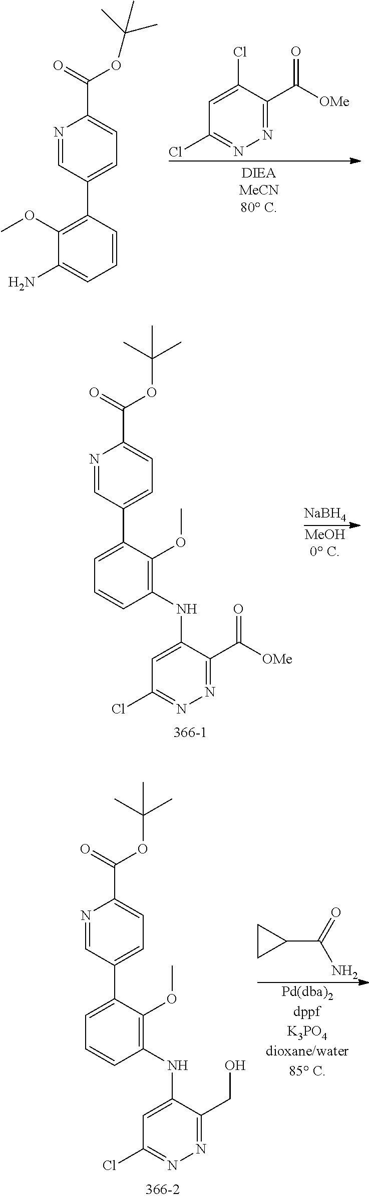

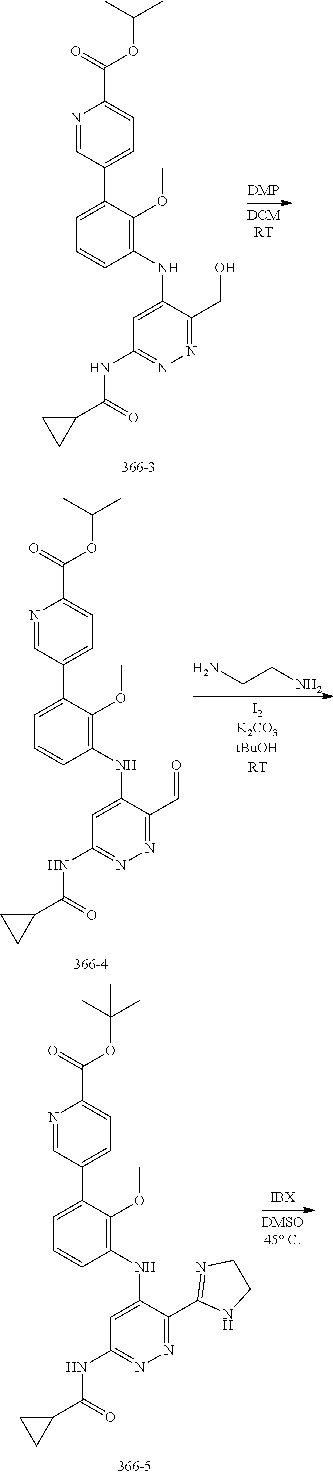

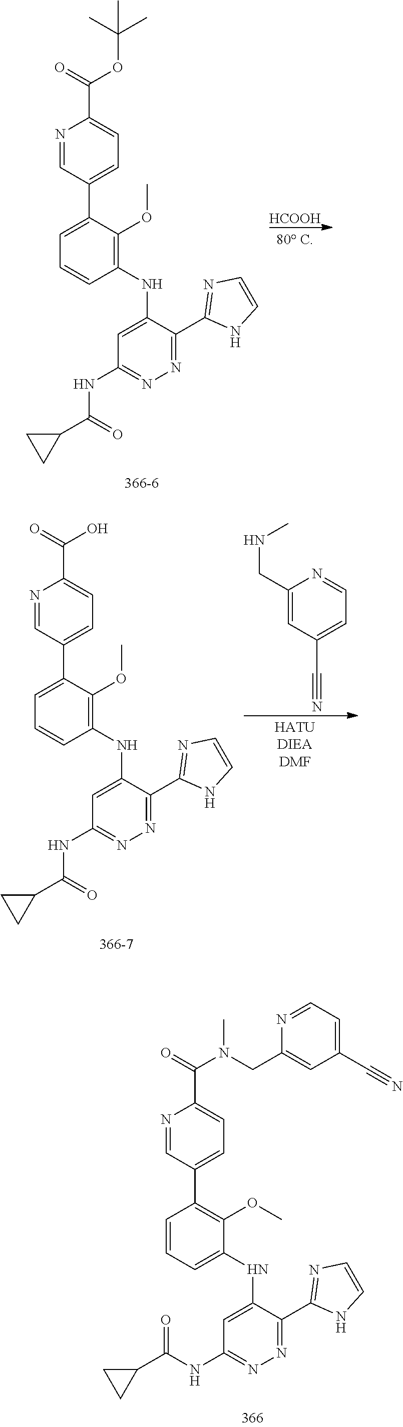 [SMILES: COc1c(Nc2cc(NC(=O)C3CC3)nnc2-c2ncc[nH]2)cccc1-c1ccc(C(=O)OC(C)(C)C)nc1 ; "HCOOH" ; COc1c(Nc2cc(NC(=O)C3CC3)nnc2-c2ncc[nH]2)cccc1-c1ccc(C(=O)O)nc1 ; CNCc1cc(C#N)ccn1 ; COc1c(Nc2cc(NC(=O)C3CC3)nnc2-c2ncc[nH]2)cccc1-c1ccc(C(=O)N(C)Cc2cc(C#N)ccn2)nc1]

Step 1: A round bottomed flask was charged with tert-butyl 5-(3-amino-2-methoxyphenyl) picolinate (4.5 g, 1.0 equiv, 15 mmol), DIEA (5.8 g, 7.8 mL, 3.0 equiv, 45 mmol), methyl 4,6-dichloropyridazine-3-carboxylate (6.2 g, 2.0 equiv, 30 mmol) and a stirbar. Acetonitrile (5 mL) was added, and the solution was stirred at 80° C. for 16 hour until the reaction was determined to be complete by LC/MS analysis. The resulting crude material was purified by silica gel chromatography. Concentration in vacuo resulted in compound 366-1 (2.7 g, 5.7 mmol, 38% yield).

m/z (ES+) [M+H]+=471.1

Step 2: A round bottomed flask was charged with compound 366-1 (1.4 g, 1.0 equiv, 3.0 mmol) and a stirbar. MeOH (25 mL) was added, and the solution was stirred at 0° C. for 5 min. NaBH₄ (0.67 g, 6.0 equiv, 18 mmol) was added and the solution was warmed to 25° C. and stirred for 120 min until the reaction was determined to be complete by LC/MS analysis. The resulting crude material was purified by flash silica gel chromatography. Concentration in vacuo resulted in compound 366-2 (1 g, 2 mmol, 80% yield.

m/z (ES+) [M+H]+=443.3

Step 3: A round bottomed flask was charged with compound 366-2 (0.96 g, 2.0 mmol, 1.0 equiv), cyclopropanecarboxamide (951 mg, 3.0 equiv, 11.2 mmol), K₃PO₄ (2.37 g, 5.59 mL, 2 molar, 3.0 equiv, 11.2 mmol) and a stirbar. 1,4-Dioxane (100 mL) and water (5.04 mL) were added, and the solution was flushed with N₂ (3×). Pd₂(dba)₃ (2.05 g, 0.6 equiv, 2.24 mmol) and dppf (2.07 g, 1.0 equiv, 3.73 mmol) were added, and the solution was flushed with N₂ (3×). The solution was stirred at 85° C. for 16 hour until the reaction was determined to be complete by LC/MS analysis. The resulting crude material was purified by prep-TLC (DCM/MeOH=15/1). Concentration in vacuo resulted in compound 366-3 (0.96 g, 2.0 mmol, 52% yield) as a yellow solid.

m/z (ES+) [M+H]+=492.0

Step 4: A round bottomed flask was charged with compound 366-3 (0.96 g, 1.0 equiv, 2.0 mmol), Dess Martin periodinane (1.7 g, 2.0 equiv, 3.9 mmol) and a stirbar. DCM (10 mL) was added, and the solution was stirred at 25° C. for 2 hour after which the resulting crude material was purified by prep-TLC (EA/PE=2/1). Concentration in vacuo resulted in compound 366-4 (0.6 g, 1 mmol, 60% yield) as a yellow solid.

m/z (ES+) [M+H]+=490.4

Step 5: A round bottomed flask was charged compound 366-4 (0.6 g, 1.0 equiv, 1 mmol), ethylenediamine (0.2 g, 0.2 mL, 3.0 equiv, 4 mmol) and a stirbar. t-BuOH (6 mL) was added, the solution was purged with N₂ (3×) and the solution was stirred at 25° C. for 30 min. Iodine (0.5 g, 1.5 equiv, 2 mmol) and K₂CO₃ (0.3 g, 1.5 equiv, 2 mmol) was added and the reaction was stirred until there was no more starting material by LC/MS analysis. The resulting crude material was purified by TLC (EA/PE=2/1). Concentration in vacuo resulted in compound 366-5 (0.35 g, 0.66 mmol, 50% yield) as a yellow solid.

m/z (ES+) [M+H]+=536.4

Step 6: A round bottomed flask was charged with compound 366-5 (0.35 g, 1.0 equiv, 0.66 mmol), 1-hydroxy-1-oxo-115-benzo[d][1,2]iodaoxol-3 (1H)-one (0.19 g, 1.0 equiv, 0.66 mmol) and a stirbar. DMSO (3 mL) was added, and the solution was stirred at 45° C. for 120 min until the reaction was determined to be complete by LC/MS analysis. The reaction mixture was then diluted with water (15 mL), and the aqueous phase was extracted with DCM (20 mL×3). The combined organic layers were washed with brine, dried over sodium sulfate, filtered, and concentrated in vacuo. Lyophilization yielded compound 366-6 (0.19 g, 0.36 mmol, 54%) as a yellow amorphous solid.

m/z (ES+) [M+H]+=528.4

Step 7: A round bottomed flask was charged with compound 366-6 (0.18 g, 1.0 equiv, 0.34 mmol), and a stirbar. Formic Acid (2 mL) was added, and the solution was stirred at 80° C. for 10 min until the reaction was determined to be complete by LC/MS analysis. Lyophilization compound 366-7 (0.15 g, 0.32 mmol, 93% yield) as a brown amorphous solid.

m/z (ES+) [M+H]+=472.2

Step 8: A round bottomed flask was charged with compound 366-7 (70 mg, 1.0 equiv, 0.15 mmol), DIEA (29 mg, 39 μL, 1.5 equiv, 0.22 mmol) and a stirbar. DMF (0.5 mL) was added and the solution stirred was at 25° C. for 30 1 min. 2-((Methylamino)methyl) isonicotinonitrile (26 mg, 1.2 Eq, 0.18 mmol) was then added and the solution was stirred at 25° C. for 120 min until the reaction was determined to be complete by LC/MS analysis. The resulting crude material was purified by flash silica gel chromatography. Concentration in vacuo resulted in compound 366 as an off-white amorphous solid.

m/z (ES+) [M+H]+=601.30.

¹H NMR (400 MHz, DMSO-d6) 13.30 (s, 1H), 11.59 (d, J=8.7 Hz, 1H), 11.21 (d, J=4.6 Hz, 1H), 8.88-8.81 (m, 1H), 8.79-8.67 (m, 1H), 8.25 (d, J=9.1 Hz, 1H), 8.15 (ddd, J=34.8, 8.1, 2.2 Hz, 1H), 7.85-7.69 (m, 3H), 7.59 (ddd, J=11.4, 7.8, 1.8 Hz, 1H), 7.41-7.27 (m, 3H), 7.25 (d, J=5.1 Hz, 1H), 4.90 (d, J=12.3 Hz, 2H), 3.41 (d, J=25.0 Hz, 3H), 3.10 (d, J=34.9 Hz, 3H), 2.16-2.05 (m, 1H), 0.83 (dd, J=7.1, 5.1 Hz, 4H).

### Example 367

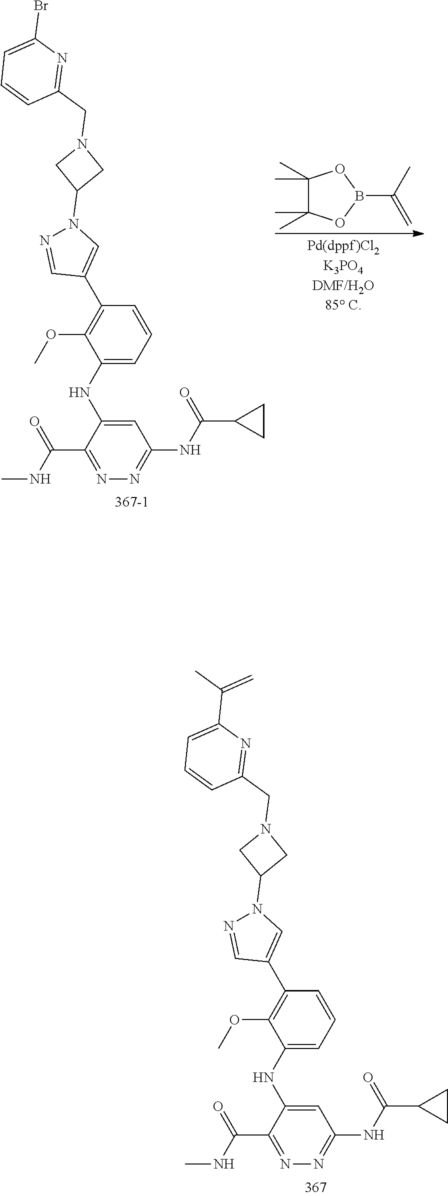 [SMILES: CNC(=O)c1nnc(NC(=O)C2CC2)cc1Nc1cccc(-c2cnn(C3CN(Cc4cccc(Br)n4)C3)c2)c1OC ; C=C(C)B1OC(C)(C)C(C)(C)O1 ; C=C(C)c1cccc(CN2CC(n3cc(-c4cccc(Nc5cc(NC(=O)C6CC6)nnc5C(=O)NC)c4OC)cn3)C2)n1]

A 2-dram vial was charged compound 367-1 (65 mg, 1.0 equiv, 0.10 mmol), isopropenyl boronic acid pinacol ester (26 mg, 29 μL, 1.5 equiv, 0.15 mmol), K₃PO₄ (2.0 equiv) and Pd(dppf)Cl₂ (15 mol %). The reaction was purged with N₂ after which 1 mL of DMF/water (3:1 v/v) was added and the reaction heated to 85° C. for 1 hr until the reaction was determined to be complete by LC/MS analysis. The reaction was then diluted with DCM and water. The organics were dry loaded on silica gel and purified using flash silica gel chromatography (gradient DCM/MeOH). Fractions containing the product were combined and concentrated to provide compound 367 as an off-white solid.

LC/MS m/z (ES+): 594.2 [M+H]+

¹H NMR (400 MHZ, DMSO) δ 11.30 (s, 1H), 10.96 (s, 1H), 9.15 (q, J=4.7 Hz, 1H), 8.37 (s, 1H), 8.14 (s, 1H), 8.01 (s, 1H), 7.74 (t, J=7.8 Hz, 1H), 7.54 (t, J=5.2 Hz, 3H), 7.51 (d, J=7.5 Hz, 1H), 7.50-7.43 (m, 2H), 7.32-7.25 (m, 2H), 7.19 (t, J=7.9 Hz, 1H), 5.90 (t, J=1.5 Hz, 1H), 5.74 (s, 1H), 5.28 (t, J=1.7 Hz, 1H), 5.11 (p, J=6.8 Hz, 1H), 3.83 (s, 3H), 3.83 (d, J=14.6 Hz, 1H), 3.60 (t, J=7.1 Hz, 2H), 3.58 (s, 3H), 2.85 (d, J=4.7 Hz, 3H), 2.13 (s, 3H), 2.11-2.01 (m, 1H), 0.86-0.77 (m, 4H).

### Example 368

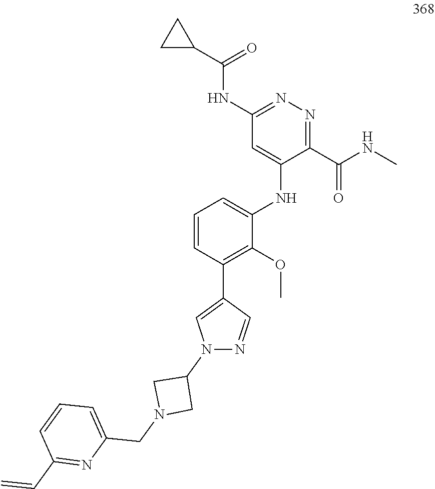 [SMILES: C=Cc1cccc(CN2CC(n3cc(-c4cccc(Nc5cc(NC(=O)C6CC6)nnc5C(=O)NC)c4OC)cn3)C2)n1]

Compound 368 was prepared in analogous fashion to compound 367 in Example 367 except that vinal boronic acid, pinacol ester was used in place of isopropenyl boronic acid pinacol ester.

LC/MS m/z (ES+): 580.1 [M+H]+

¹H NMR (400 MHZ, DMSO) δ 11.30 (s, 1H), 10.96 (s, 1H), 9.16 (d, J=4.9 Hz, 1H), 8.37 (d, J=0.8 Hz, 1H), 8.14 (s, 1H), 8.01 (d, J=0.7 Hz, 1H), 7.74 (t, J=7.7 Hz, 1H), 7.47 (dd, J=7.8, 1.6 Hz, 1H), 7.37 (dd, J=7.7, 1.0 Hz, 1H), 7.29 (dd, J=7.9, 1.6 Hz, 2H), 7.19 (t, J=7.9 Hz, 1H), 6.78 (dd, J=17.4, 10.7 Hz, 1H), 6.21 (dd, J=17.5, 1.7 Hz, 1H), 5.44 (dd, J=10.7, 1.7 Hz, 1H), 5.11 (p, J=6.9 Hz, 1H), 3.85-3.77 (m, 4H), 3.63-3.57 (m, 2H), 3.58 (s, 3H), 2.85 (d, J=4.8 Hz, 3H), 2.10-2.03 (m, 1H), 0.84-0.77 (m, 4H).

### Example 370

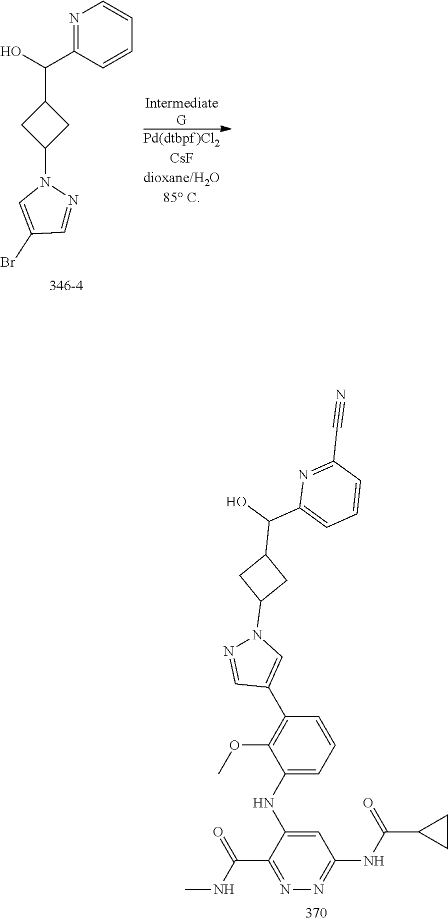 [SMILES: OC(c1ccccn1)C1CC(n2cc(Br)cn2)C1 ; "IntermediateG" ; "Pd(dtbpf)Cl2CsFdioxane/H2O85° C." ; CNC(=O)c1nnc(NC(=O)C2CC2)cc1Nc1cccc(-c2cnn(C3CC(C(O)c4cccc(C#N)n4)C3)c2)c1OC]

Compound 346-4 (50 mg, 1.0 equiv, 0.19 mmol), Intermediate G (89 mg, 1.0 equiv, 0.19 mmol), cesium fluoride (29 mg, 7.0 μL, 1.0 equiv, 0.19 mmol), water (0.1 mL), 1,4-dioxane (0.5 mL) were added to 8 mL vial, and the reaction was purged with nitrogen. 1,1′-Bis(di-t-butylphosphino) ferrocene palladium dichloride (25 mg, 0.2 equiv, 38 μmol) was added to the solution under nitrogen atmosphere. The reaction mixture was stirred at 85° C. for 2 hours. The reaction mixture was diluted with H₂O (30 mL), and the aqueous phase was extracted with DCM (3×50 mL) three times. The combined organic layers were washed with brine, dried over sodium sulfate, filtered, and concentrated in vacuo. The crude products was purified by prep-HPLC(Column: XBridge Prep OBD C18 Column, gradient 10 mM NH₄HCO₃ in water/MeCN), and concentrated to provide compound 370 (5 mg, 9 μmol, 5% yield) as an off-white amorphous solid.

m/z (ES+) [M+H]+=569.4

¹H NMR (400 MHZ, DMSO-d6) 11.31 (d, J=2.5 Hz, 1H), 10.98 (d, J=7.5 Hz, 1H), 9.17 (q, J=3.6, 2.5 Hz, 1H), 8.54-8.46 (m, 1H), 8.26 (d, J=6.6 Hz, 1H), 8.16 (d, J=5.4 Hz, 1H), 7.96 (d, J=1.8 Hz, 1H), 7.78 (td, J=7.6, 1.8 Hz, 1H), 7.57-7.42 (m, 2H), 7.34-7.13 (m, 3H), 5.56 (d, J=5.1 Hz, 1H), 4.78-4.66 (m, 1H), 4.62 (t, J=4.6 Hz, 1H), 3.59 (d, J=4.9 Hz, 3H), 2.87 (dd, J=4.8, 1.8 Hz, 3H), 2.51-2.42 (m, 2H), 2.37-2.22 (m, 2H), 2.08 (q, J=6.1 Hz, 1H), 0.82 (q, J=2.6 Hz, 4H).

### Example 371

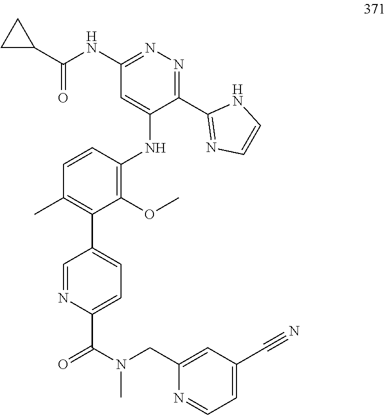 [SMILES: COc1c(Nc2cc(NC(=O)C3CC3)nnc2-c2ncc[nH]2)ccc(C)c1-c1ccc(C(=O)N(C)Cc2cc(C#N)ccn2)nc1]

Compound 371 was prepared using analogous procedures as those in Example 366 except that tert-butyl 5-(3-amino-2-methoxy-6-methylphenyl) picolinate was used as starting material instead of tert-butyl 5-(3-amino-2-methoxyphenyl) picolinate.

LC/MS m/z (ES+): 615.3 [M+H]+

¹H NMR (400 MHZ, DMSO-d6) 13.25 (s, 1H), 11.41 (d, J=10.6 Hz, 1H), 11.18 (d, J=5.0 Hz, 1H), 8.81 (ddd, J=30.3, 4.9, 1.0 Hz, 1H), 8.52 (ddd, J=61.6, 2.1, 0.9 Hz, 1H), 8.19 (d, J=9.7 Hz, 1H), 8.00-7.65 (m, 4H), 7.47 (dd, J=11.6, 8.2 Hz, 1H), 7.36-7.30 (m, 1H), 7.27-7.17 (m, 2H), 4.90 (d, J=12.5 Hz, 2H), 3.30 (d, J=30.9 Hz, 3H), 3.11 (d, J=22.8 Hz, 3H), 2.14-2.03 (m, 4H), 0.83 (t, J=6.3 Hz, 4H).

### Example 372

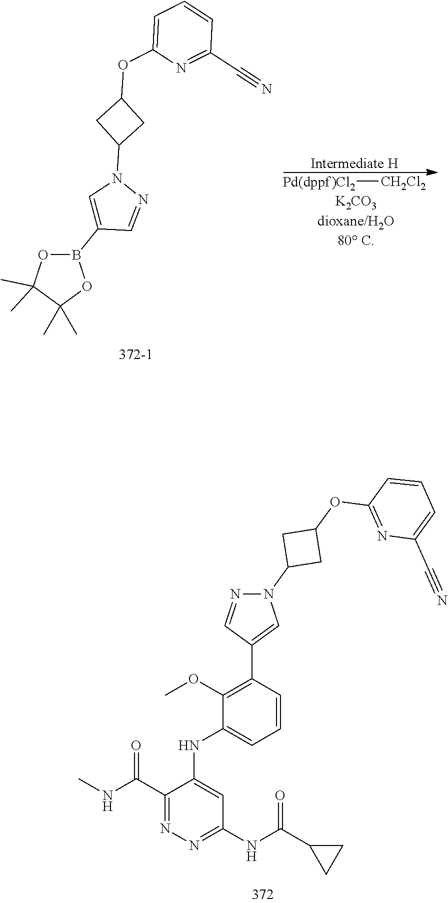

Intermediate H (115 mg, 1.0 equiv, 273 μmol), and compound 372-1 (100 mg, 1.0 equiv, 273 μmol), potassium carbonate (113 mg, 47.7 μL, 3.0 equiv, 819 μmol), 1,4-dioxane (2.5 mL), and waterer (0.5 mL) were added to a 40 ml vial, and the reaction was purged with nitrogen. PdCl₂ (dppf)-CH₂Cl₂ (44.6 mg, 0.2 equiv, 54.6 μmol) was added to the solution under nitrogen atmosphere. The reaction mixture was stirred at 80° C. for 2 hours. The reaction was diluted with water (10 mL) and extracted with DCM (3×10 mL) three times. The organic layer was washed with brine, dried over Na₂SO₄ and evaporated.

The resulted solution was purified using C18 flash chromatography (gradient: MeCN/water) Concentration in vacuo resulted compound 372 (5.1 mg, 9 μmol, 3% yield) as an off-white amorphous solid.

m/z (ES+) [M+H]+=580.3

¹H NMR (400 MHZ, DMSO-d6) 11.31 (s, 1H), 10.97 (s, 1H), 9.16 (q, J=4.9 Hz, 1H), 8.32 (s, 1H), 8.15 (s, 1H), 8.02 (s, 1H), 7.97 (t, J=7.9 Hz, 1H), 7.68 (d, J=7.2 Hz, 1H), 7.51-7.44 (m, 1H), 7.33-7.15 (m, 3H), 5.10 (p, J=7.3 Hz, 1H), 4.77 (p, J=8.1 Hz, 1H), 3.59 (s, 3H), 3.12-3.01 (m, 2H), 2.87 (d, J=4.8 Hz, 3H), 2.69 (dt, J=11.9, 8.8 Hz, 2H), 2.08 (p, J=6.2 Hz, 1H), 0.83 (dd, J=7.4, 3.7 Hz, 4H).

### Example 375

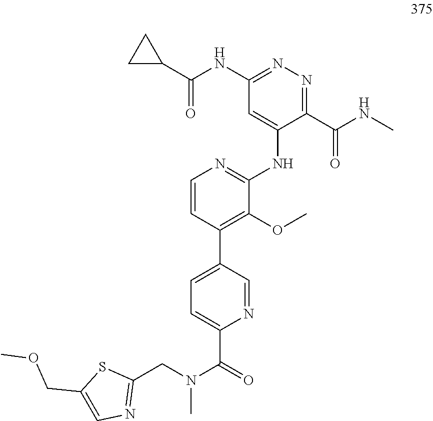

Compound 375 was prepared using analogous conditions as those in Example 240.

m/z (ES+) [M+H]+=618.3

¹H NMR (400 MHz, DMSO-d6) 12.46 (s, 1H), 11.38 (s, 1H), 9.86 (d, J=2.7 Hz, 1H), 9.29 (d, J=5.1 Hz, 1H), 8.89 (dd, J=15.2, 2.2 Hz, 1H), 8.27 (ddd, J=8.1, 4.1, 2.2 Hz, 1H), 8.21 (t, J=5.3 Hz, 1H), 7.82 (dd, J=17.5, 8.1 Hz, 1H), 7.69 (d, J=15.3 Hz, 1H), 7.20 (d, J=5.2 Hz, 1H), 4.99 (d, J=16.8 Hz, 2H), 4.62 (d, J=11.9 Hz, 2H), 3.53 (d, J=8.7 Hz, 3H), 3.12 (s, 3H), 3.07 (s, 3H), 2.88 (d, J=4.7 Hz, 3H), 2.24-2.05 (m, 1H), 0.89 (t, J=7.2 Hz, 4H).

### Example 376

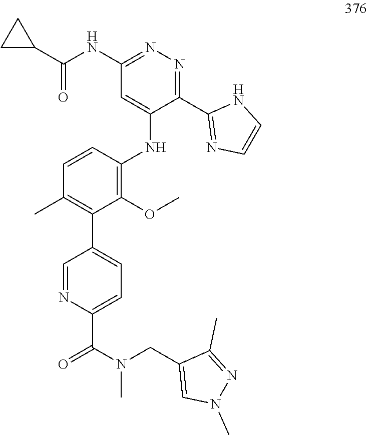 [SMILES: COc1c(Nc2cc(NC(=O)C3CC3)nnc2-c2ncc[nH]2)ccc(C)c1-c1ccc(C(=O)N(C)Cc2cn(C)nc2C)nc1]

Compound 376 was prepared using analogous procedures as those in Example 366 except that tert-butyl 5-(3-amino-2-methoxy-6-methylphenyl) picolinate was used as starting material instead of tert-butyl 5-(3-amino-2-methoxyphenyl) picolinate.

LC/MS m/z (ES+): 607.4 [M+H]+

¹H NMR (400 MHZ, DMSO-d6) 13.26 (s, 1H), 11.41 (s, 1H), 11.18 (s, 1H), 8.57 (dd, J=22.0, 2.1 Hz, 1H), 8.25-8.18 (m, 1H), 7.94 (td, J=7.9, 2.2 Hz, 1H), 7.68 (dd, J=10.5, 8.0 Hz, 1H), 7.60 (d, J=9.2 Hz, 1H), 7.48 (dd, J=8.2, 2.0 Hz, 1H), 7.33 (s, 1H), 7.22 (d, J=8.7 Hz, 2H), 4.48 (d, J=23.2 Hz, 2H), 3.73 (d, J=14.7 Hz, 3H), 3.32 (s, 3H), 2.91 (d, J=24.3 Hz, 3H), 2.18-1.90 (m, 7H), 0.83 (d, J=6.2 Hz, 4H).

### Example 377

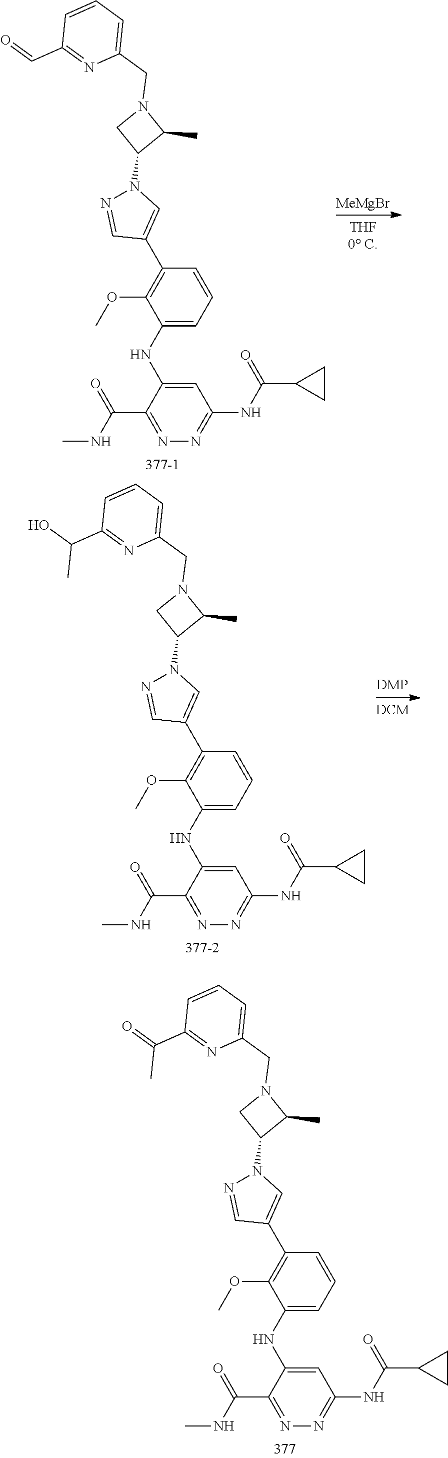 [SMILES: "MeMgBr" ; CNC(=O)c1nnc(NC(=O)C2CC2)cc1Nc1cccc(-c2cnn([C@@H]3CN(Cc4cccc(C=O)n4)[C@H]3C)c2)c1OC ; CNC(=O)c1nnc(NC(=O)C2CC2)cc1Nc1cccc(-c2cnn([C@@H]3CN(Cc4cccc(C(C)O)n4)[C@H]3C)c2)c1OC ; CNC(=O)c1nnc(NC(=O)C2CC2)cc1Nc1cccc(-c2cnn([C@@H]3CN(Cc4cccc(C(C)=O)n4)[C@H]3C)c2)c1OC]

Compound 377-1 was prepared according to analogous procedures from Example 310.

Step 1: In a 40 ml vial was charged compound 377-1 (100 mg, 1.0 equiv, 168 μmol) in dry THF. The reaction was cooled to 0° C. after which methyl magnesium bromide (50.1 mg, 140 μL, 3 molar, 2.5 equiv, 420 μmol) was added and stirred for 30 min after which there was complete conversion to the desired by LC/MS analysis. The reaction was quenched with aq NH₄Cl and extracted with MTBE. Organics were dried and concentrated to provide compound 377-2 that was used directly in the next step.

Step 2: Compound 377-2 was dissolved in DCM (10 m) followed by the addition of Dess Martin Periodinane (107 mg, 1.5 equiv, 252 μmol) and stirred for 2 hrs after which complete conversion to the desired was observed by LC/MS. The reaction quenched with aq Na₂S2O₃ and extracted with MTBE. The organics were dry loaded on silica purified by flash silica gel chromatography (gradient: DCM/MeOH). Product fractions were combined and concentrated to provide compound 377 as a white solid.

LC/MS m/z (ES+): 610.1 [M+H]+

¹H NMR (500 MHZ, DMSO) δ 11.29 (s, 1H), 10.97 (s, 1H), 9.15 (q, J=4.9 Hz, 1H), 8.45 (s, 1H), 8.15 (s, 1H), 7.99 (s, 1H), 7.98 (t, J=7.7 Hz, 1H), 7.83 (dd, J=7.7, 1.1 Hz, 1H), 7.73 (dd, J=7.8, 1.1 Hz, 1H), 7.49 (dd, J=7.9, 1.6 Hz, 1H), 7.30 (dd, J=7.9, 1.5 Hz, 1H), 7.20 (t, J=7.9 Hz, 1H), 5.06 (td, J=7.2, 2.4 Hz, 1H), 4.02-3.93 (m, 2H), 3.87-3.78 (m, 2H), 3.60 (t, J=8.5 Hz, 1H), 3.58 (s, 3H), 2.86 (d, J=4.8 Hz, 3H), 2.63 (s, 3H), 2.53 (s, 1H), 2.42 (s, 1H), 2.11-2.02 (m, 1H), 1.22 (s, 1H), 0.86-0.78 (m, 4H), 0.66 (d, J=6.3 Hz, 3H).

### Example 379

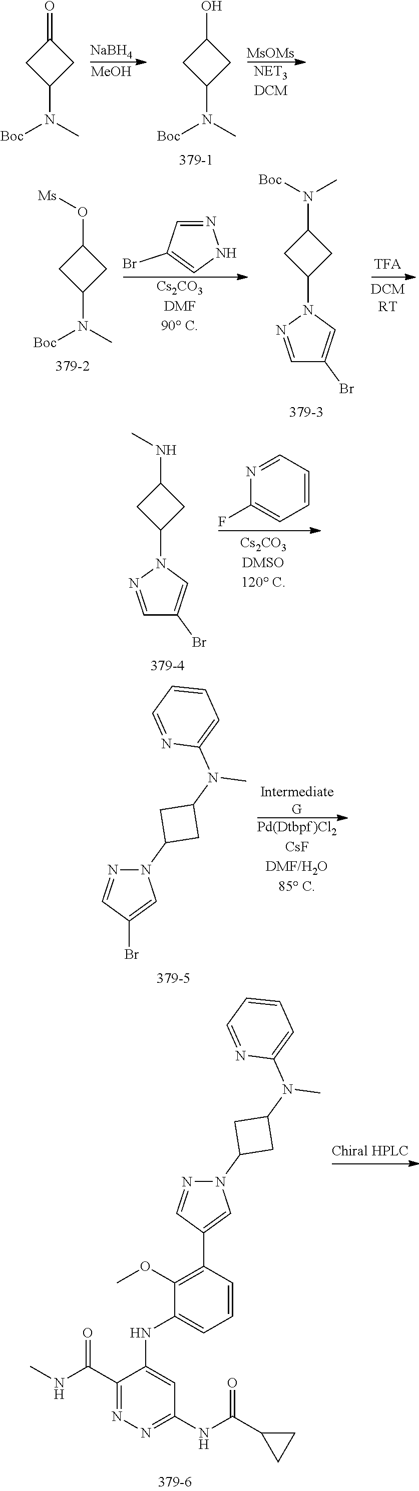

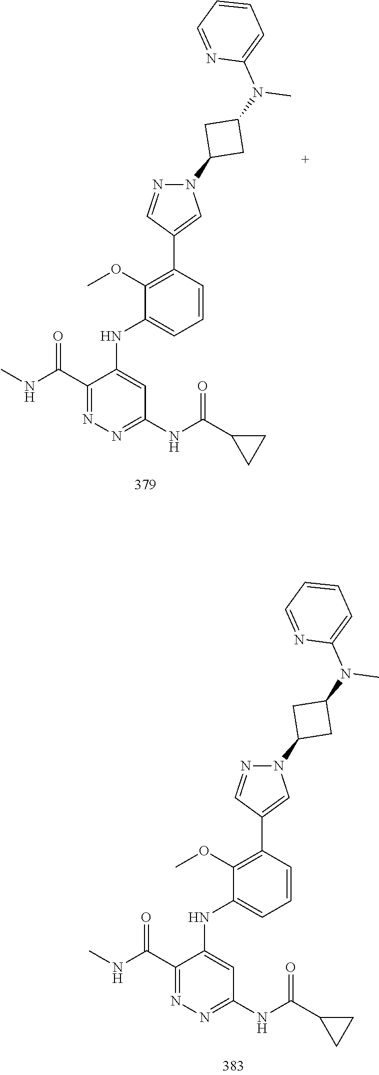

Step 1: A round bottomed flask was charged with tert-butyl methyl (3-oxocyclobutyl) carbamate (2 g, 1.0 equiv, 0.01 mol), NaBH₄ (1 g, 1 mL, 3.0 equiv, 0.03 mol) MeOH (5 mL) were added, and the solution was stirred at 25° C. for 2 h until the reaction was determined to be complete by LC/MS analysis. The reaction mixture was diluted with water (500 mL), and the aqueous phase was extracted with EA (300 mL) three times. The combined organic layers were washed with saturated NaCl, dried over sodium sulfate, filtered, and concentrated in vacuo. The resulting solution was purified using C18 flash chromatography (gradient: MeCN/water) Concentration in vacuo resulted in compound 379-1 as a off-white solid.

m/z (ES+) [M+H]+=146.1

Step 2: A round bottomed flask was charged with compound 379-1 (800 mg, 1.0 equiv, 3.97 mmol), dimesyl anhydride (1.1 equiv), triethylamine (2.01 g, 2.77 mL, 5.0 equiv, 19.9 mmol) and a stir bar. DCM (5 mL) was added, and the solution was stirred at 25° C. for 2 hour until the reaction was determined to be complete by LC/MS analysis. The reaction mixture was diluted with water (500 mL), and the aqueous phase was extracted with EA (300 mL) three times. The combined organic layers were washed with saturated NaCl dried over sodium sulfate, filtered, and concentrated in vacuo. The resulting crude material was purified by flash silica gel chromatography (gradient: heptanes/ethyl acetate). Concentration in vacuo resulted compound 379-2 as a off-white solid.

m/z (ES+) [M+H]+=302.1

Step 3: A round bottomed flask was charged with 379-2 (1 g, 1.0 equiv, 4 mmol), cesium carbonate (3 g, 0.9 mL, 3.0 equiv, 0.01 mol), 4-bromo-1H-pyrazole (0.6 g, 1.2 equiv, 4 mmol) and a stir bar. DMF (3 mL) was then added, and the solution was stirred at 90° C. for 6 hour until the reaction was determined to be complete by LC/MS analysis. The reaction mixture was then diluted with water (100 mL), and the aqueous phase was extracted with EA (100 mL) three times. The combined organic layers were washed with saturated NaCl dried over sodium sulfate, filtered, and concentrated in vacuo. The resulted solution was purified using C18 flash chromatography (gradient: MeCN/water). Concentration in vacuo resulted in compound 379-3 as a off-white solid.

m/z (ES+) [M+H]+=330.2

Step 4: A round bottomed flask was charged with compound 379-3 (500 mg, 1.0 equiv, 1.51 mmol), trifluoroacetic acid (345 mg, 230 μL, 2.0 equiv, 3.03 mmol) and a stir bar. DCM (2 mL) was added, and the solution was stirred at 25° C. for 2 hour until the reaction was determined to be complete by LC/MS analysis. The reaction mixture was diluted with water (100 mL), and the aqueous phase was extracted with EA (100 mL) three times. The combined organic layers were washed with saturated NaCl dried over sodium sulfate, filtered, and concentrated in vacuo. The resulted solution was purified using C18 flash chromatography (gradient: MeCN/water. Concentration in vacuo resulted in compound 379-4 as a off-white solid.

m/z (ES+) [M+H]+=231.9

Step 5: A round bottomed flask was charged with compound 379-4 (500 mg, 1.0 equiv, 2.17 mmol), cesium carbonate (1.42 g, 348 μL, 2.0 equiv, 4.35 mmol), 2-fluoropyridine (316 mg, 1.5 equiv, 3.26 mmol) and a stir bar. DMSO (3 mL) was added, and the solution was stirred at 120° C. for 12 hours until the reaction was determined to be complete by LC/MS analysis. The reaction mixture was diluted with water (500 mL), and the aqueous phase was extracted with EA (300 mL) three times. The combined organic layers were washed with saturated NaCl dried over sodium sulfate, filtered, and concentrated in vacuo. The resulting crude material was purified by silica gel chromatography (gradient: heptanes/ethyl acetate). Concentration in vacuo resulted in compound 379-5 as a off-white solid.

m/z (ES+) [M+H]+=307.0

Step 6: A round bottomed flask was charged with compound 379-5 (80 mg, 1.0 equiv, 0.26 mmol), 1,1′-bis(di-t-butylphosphino) ferrocene palladium dichloride (34 mg, 0.2 equiv, 52 μmol), Intermediate G (0.12 g, 1.0 equiv, 0.26 mmol), cesium fluoride (0.12 g, 29 μL, 3.0 equiv, 0.78 mmol) and a stirbar. DMF (0.8 mL), H₂O (0.2 mL) was added, and the solution was stirred at 80° C. for 2 hour until the reaction was determined to be complete by LC/MS analysis. The reaction mixture was diluted with water (50 mL), and the aqueous phase was extracted with EA (30 mL) three times. The combined organic layers were washed with sat NaCl dried over sodium sulfate, filtered, and concentrated in vacuo. The resulted solution was purified using C18 flash chromatography (gradient: MeCN/water). Concentration in vacuo resulted in compound 379-6 as a off-white solid.

m/z (ES+) [M+H]+=568.3

The cis and trans diasteromers were separated by chiral HPLC to provide compound 379 as the trans diastereomer:

¹H NMR (400 MHZ, Chloroform-d) 11.15 (s, 1H), 9.24 (s, 1H), 8.31 (s, 1H), 8.22 (ddd, J=5.0, 2.0, 0.8 Hz, 1H), 8.17-8.12 (m, 1H), 8.05 (d, J=0.7 Hz, 1H), 8.00 (d, J=0.7 Hz, 1H), 7.52 (ddd, J=8.9, 7.1, 2.0 Hz, 1H), 7.36 (ddd, J=9.2, 7.9, 1.6 Hz, 2H), 7.22 (t, J=7.9 Hz, 1H), 6.67-6.56 (m, 2H), 5.24-5.13 (m, 1H), 4.95-4.87 (m, 1H), 3.69 (s, 3H), 3.17 (s, 3H), 3.08 (d, J=5.1 Hz, 3H), 3.01-2.83 (m, 4H), 1.78 (tt, J=7.8, 4.5 Hz, 1H), 1.18-1.11 (m, 2H), 0.97-0.91 (m, 2H).

The cis and trans diasteromers were separated by chiral HPLC to provide compound 381 as the cis diastereomer:

¹H NMR (400 MHZ, Chloroform-d) 11.16 (s, 1H), 9.32 (s, 1H), 8.31 (d, J=2.7 Hz, 1H), 8.22 (dd, J=5.2, 1.9 Hz, 1H), 8.14 (s, 1H), 7.97 (d, J=6.8 Hz, 2H), 7.54 (t, J=8.0 Hz, 1H), 7.34 (ddd, J=15.1, 7.9, 1.6 Hz, 2H), 7.21 (t, J=7.9 Hz, 1H), 6.63 (ddd, J=12.4, 8.0, 4.1 Hz, 2H), 5.04-4.91 (m, 1H), 4.66-4.53 (m, 1H), 3.67 (s, 3H), 3.14 (d, J=2.7 Hz, 3H), 3.07 (d, J=5.0 Hz, 3H), 2.96 (s, 2H), 2.96-2.81 (m, 2H), 1.43 (d, J=6.3 Hz, 1H), 1.20-1.09 (m, 2H), 0.94 (dq, J=7.4, 4.0 Hz, 2H).

### Example 392

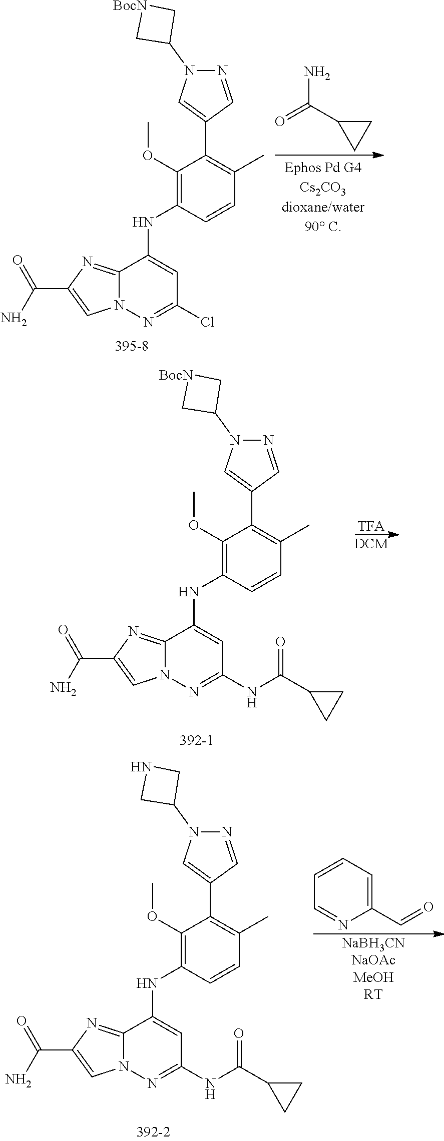

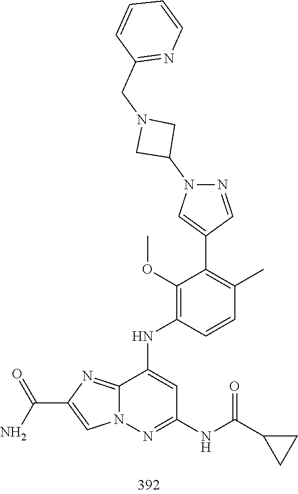 [SMILES: COc1c(Nc2cc(NC(=O)C3CC3)nn3cc(C(N)=O)nc23)ccc(C)c1-c1cnn(C2CN(Cc3ccccn3)C2)c1]

Step 1: To a stirred solution of compound 395-8 (100 mg, 1.0 equiv, 181 μmol), cyclopropanecarboxamide (23.1 mg, 1.5 equiv, 271 μmol), Cs₂CO₃ (118 mg, 2.0 equiv, 362 μmol) in 1,4-dioxane (0.4 mL) and H₂O (0.1 mL) were added EPhos Pd G4 (24.9 mg, 0.15 equiv, 27.1 μmol) and the resulting solution was stirred at 90° C. for 1 h under nitrogen atmosphere. The resulting mixture was concentrated under reduced pressure and was dissolved in DCM. The crude product obtained was purified by flash silica gel column chromatography (gradient: DCM/MeOH) to provide compound 392-1 as a yellow solid.

m/z (ES+) [M+H]+=602.3

Step 2: To a stirred solution of compound 392-1 (25 mg, 1.0 equiv, 42 μmol) in DCM (1 mL) was added TFA (0.4 g, 0.3 mL, 9.0 equiv, 4 mmol) and the resulting solution was stirred at room temperature for 1 h. The resulting mixture was concentrated under reduced pressure. This resulted in compound 392-2 (35 mg, 70 μmol, 170%) as a light yellow oil.

m/z (ES+) [M+H]+=502.4

Step 3: To a stirred solution of compound 391-2 (100 mg, 1.0 equiv, 199 μmol), picolinaldehyde (42.7 mg, 2.0 equiv, 399 μmol), in MeOH (10 mL) at room temperature. Sodium acetate (3.0 equiv), followed by portion-wise addition of sodium cyanoborohydride (2.0 equiv). The resulting mixture was stirred at room temperature for 2 h. The reaction was quenched with water and extracted with EA. The organic layer was washed with brine, dried over Na₂SO₄ and evaporated. The crude material was purified by HPLC(Column: Xselect CSH C18 OBD, gradient: water (0.05% TFA)/MeCN). This resulted in compound 392 a white amorphous solid.

m/z (ES+) [M+H]+=593.3

### Example 393

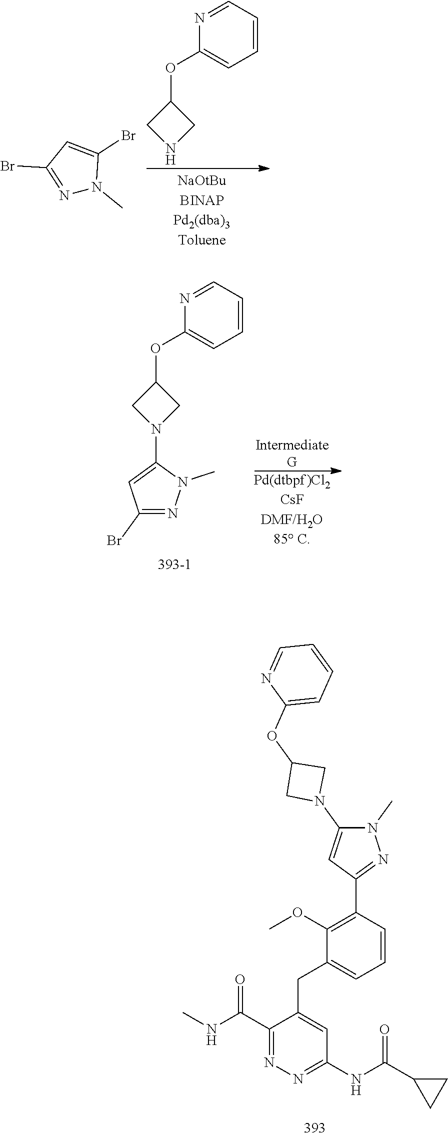

Step 1: A round bottomed flask was charged with 3,5-dibromo-1-methyl-1H-pyrazole (500 mg, 1.0 equiv, 2.08 mmol), NaOtBu, (601 mg, 674 μL, 3.0 equiv, 6.25 mmol) 2-(azetidin-3-yloxy)pyridine (313 mg, 1.0 equiv, 2.08 mmol), Pd₂(dba)₃ (382 mg, 0.2 equiv, 417 μmol), BINAP (649 mg, 0.5 equiv, 1.04 mmol) and a stir bar. Toluene (1 mL) was added, and the solution was stirred at 120° C. for 12 hour until the reaction was determined to be complete by LC/MS analysis. The reaction mixture was diluted with water (100 mL), and the aqueous phase was extracted with ethyl acetate (100 mL) three times. The combined organic layers were washed with saturated NaCl, dried over sodium sulfate, filtered, and concentrated in vacuo.

The resulted solution was purified using C18 flash chromatography (gradient: MeCN/water). Concentration in vacuo resulted in compound 393-1 as a off-white solid.

m/z (ES+) [M+H]+=310.9

Step 2: A round bottomed flask was charged compound 393-1 (100 mg, 1.0 equiv, 323 μmol), 1,1′-bis(di-t-butylphosphino) ferrocene palladium dichloride (42.2 mg, 0.2 equiv, 64.7 μmol), Intermediate G (151 mg, 1.0 equiv, 323 μmol), cesium fluoride (147 mg, 35.8 μL, 3.0 equiv, 970 μmol) and a stir bar. DMF (1 mL) and H₂O (0.2 mL) was added, the solution was under an N₂ atmosphere and the solution was stirred at 85° C. for 2 hour until the reaction was determined to be complete by LC/MS analysis. The reaction mixture was diluted with water (50 mL), and the aqueous phase was extracted with ethyl acetate (30 mL) three times. The combined organic layers were washed with saturated NaCl dried over sodium sulfate, filtered, and concentrated in vacuo. The resulted solution was purified using C18 flash chromatography (gradient: MeCN/water). Concentration in vacuo resulted in compound 393 as a off-white solid.

m/z (ES+) [M+H]+=570.3

¹H NMR (400 MHZ, DMSO-d6) 11.31 (s, 1H), 10.96 (s, 1H), 9.16 (q, J=4.7 Hz, 1H), 8.18 (ddd, J=5.0, 2.0, 0.8 Hz, 1H), 8.14 (s, 1H), 7.77 (ddd, J=8.3, 7.2, 2.0 Hz, 1H), 7.65 (dd, J=7.9, 1.6 Hz, 1H), 7.36 (dd, J=7.9, 1.6 Hz, 1H), 7.19 (t, J=7.9 Hz, 1H), 7.04 (ddd, J=7.1, 5.1, 1.0 Hz, 1H), 6.92 (dd, J=8.3, 1.0 Hz, 1H), 6.12 (s, 1H), 5.45 (tt, J=6.3, 4.7 Hz, 1H), 4.42-4.34 (m, 2H), 3.90 (dd, J=8.7, 4.8 Hz, 2H), 3.63 (d, J=16.3 Hz, 6H), 2.87 (d, J=4.8 Hz, 3H), 2.08 (p, J=6.2 Hz, 1H), 0.86-0.79 (m, 4H).

### Example 395

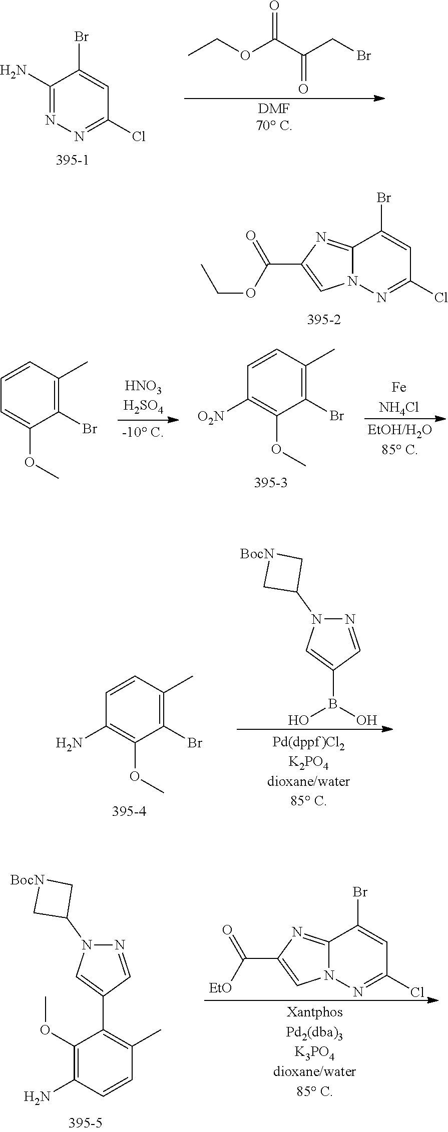

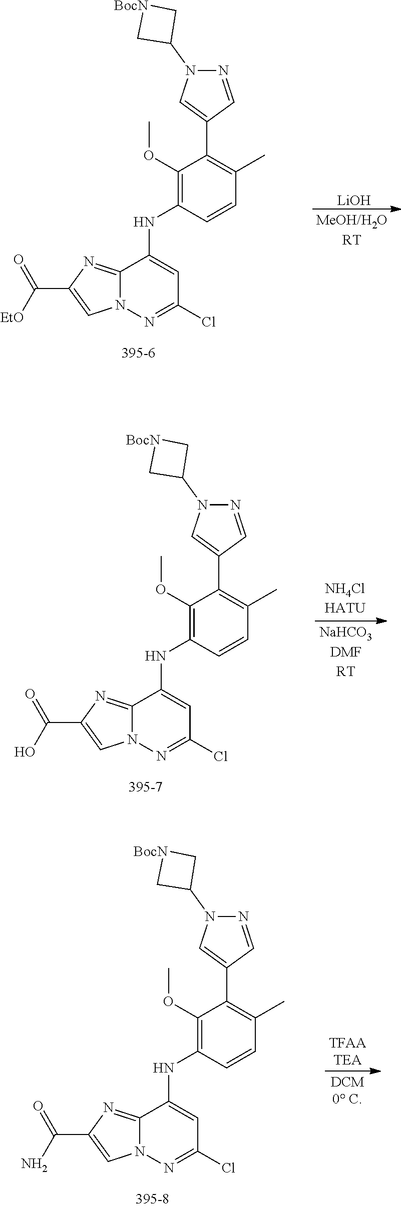 [SMILES: CCOC(=O)c1cn2nc(Cl)cc(Nc3ccc(C)c(-c4cnn(C5CN(C(=O)OC(C)(C)C)C5)c4)c3OC)c2n1 ; "LiOH" ; COc1c(Nc2cc(Cl)nn3cc(C(=O)O)nc23)ccc(C)c1-c1cnn(C2CN(C(=O)OC(C)(C)C)C2)c1 ; COc1c(Nc2cc(Cl)nn3cc(C(N)=O)nc23)ccc(C)c1-c1cnn(C2CN(C(=O)OC(C)(C)C)C2)c1]

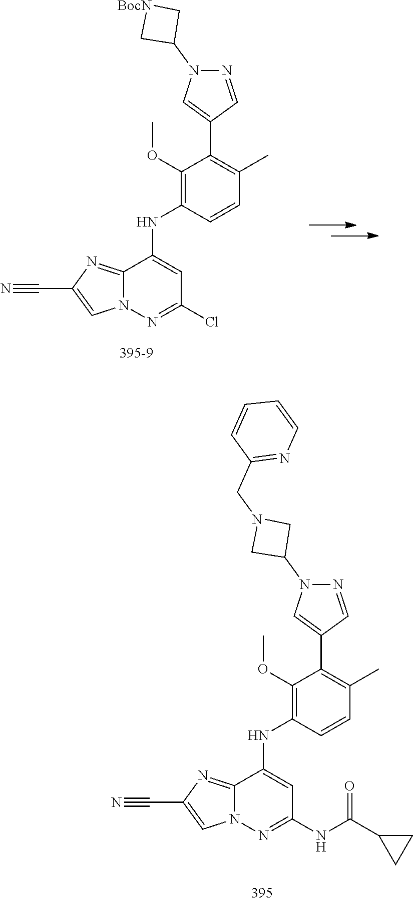 [SMILES: COc1c(Nc2cc(Cl)nn3cc(C#N)nc23)ccc(C)c1-c1cnn(C2CN(C(=O)OC(C)(C)C)C2)c1 ; COc1c(Nc2cc(NC(=O)C3CC3)nn3cc(C#N)nc23)ccc(C)c1-c1cnn(C2CN(Cc3ccccn3)C2)c1]

Step 1: A stirred solution of 4-bromo-6-chloropyridazin-3-amine (2 g, 1.0 equiv, 0.01 mol) and ethyl 3-bromo-2-oxopropanoate (3 g, 1.5 equiv, 0.01 mol) in DMF (20 mL) was heated to 70° C. The resulting mixture was stirred at 70° C. for 12 hours. The resulted solution was purified using C18 flash chromatography (gradient: MeCN/water) This resulted in compound 395-2 (2 g, 7 mmol, 70% yield) as a yellow solid.

m/z (ES+) [M+H]+=306.0

Step 2: To a solution of 2-bromo-1-methoxy-3-methylbenzene (10 g, 1 equiv, 50 mmol) in DCM (20 mL) was added H₂SO₄ (98 g, 53 mL, 20 equiv, 0.99 mol) and nitric acid (31 g, 26 mL, 10 equiv, 0.50 mol) at −10° C., and the resulting mixture was stirred at −10° C. The reaction was quenched with water and extracted with DCM. The organic layer was washed with brine, dried over Na₂SO₄ and evaporated. This resulted in compound 395-3 (10 g, 41 mmol, 82% yield) as a yellow solid.

m/z (ES+) [M+H]+=247.0

Step 3: To a solution of compound 395-3 (10 g, 1.0 equiv, 41 mmol) in EtOH (80 mL) and H₂O (20 mL) was added ammonium chloride (4.3 g, 3.0 mL, 2.0 equiv, 81 mmol) and iron (1.1 g, 0.14 mL, 0.5 equiv, 20 mmol) at room temperature, after which the resulting mixture was stirred at 85° C. for 12 h. The reaction was quenched with water and extracted with DCM. The organic layer was washed with brine, dried over Na₂SO₄ and evaporated. The resulting solution was purified using C18 flash chromatography (gradient: MeCN/water) to provide compound 395-4 (300 mg, 1.39 mmol, 3.4%) as a yellow solid.

m/z (ES+) [M+H]+=218.1

Step 4: To a stirred solution of compound 395-4 (300 mg, 1.0 equiv, 1.39 mmol), (1-(1-(tert-butoxycarbonyl) azetidin-3-yl)-1H-pyrazol-4-yl) boronic acid (445 mg, 1.2 equiv, 1.67 mmol), potassium phosphate, tribasic (884 mg, 345 μL, 3 equiv, 4.17 mmol) in 1,4-dioxane (4 mL) and H₂O (1 mL) were added followed by the addition of PdCl₂ (dppf) (203 mg, 0.2 equiv, 278 μmol) and the resulting solution was stirred at 85° C. for 2 h under nitrogen. The resulting mixture was concentrated under reduced pressure and was dissolved in DCM. The resulted solution was purified using C18 flash chromatography (gradient: MeCN/water) This resulted in compound 395-5 (500 mg, 1.39 mmol, 100% yield) as a yellow solid.

m/z (ES+) [M+H]+=359.30

Step 5: To a stirred solution of compound 395-5 (1.8 g, 1.0 equiv, 5.0 mmol), compound 395-2 (1.8 g, 1.2 equiv, 6.0 mmol), potassium phosphate, tribasic (3.2 g, 1.2 mL, 3.0 equiv, 15 mmol) in 1,4-dioxane (0.5 mL) and H₂O (0.1 mL) were added Pd₂(dba)₃ (0.92 g, 0.2 equiv, 1.0 mmol) and xantphos (0.58 g, 0.2 Eq, 1.0 mmol), the resulting solution was stirred at 85° C. for 2 h under nitrogen atmosphere. The resulting mixture was concentrated under reduced pressure and was dissolved in DMF. The resulted solution was purified using C18 flash chromatography (gradient: MeCN/water). This resulted in compound 395-6 (1.3 g, 2.2 mmol, 44% yield) as a yellow solid.

m/z (ES+) [M+H]+=582.4

Step 6: To a stirred solution of compound 395-6 (1 g, 1.0 equiv, 2 mmol), LiOH (0.04 g, 1.0 equiv, 2 mmol), in MeOH (0.4 mL) and H₂O (0.1 mL), and the resulting solution was stirred at room temperature for 2 h. The reaction was quenched with water and extracted with EA. The organic layer was washed with brine, dried over Na₂SO₄ and evaporated. The resulting mixture was concentrated under reduced pressure. This resulted in compound 395-7 (600 mg, 1.08 mmol, 60%) as a yellow solid.

m/z (ES+) [M+H]+=554.4

Step 7: A solution of compound 395-7 (800 mg, 1.0 equiv, 1.44 mmol), ammonium chloride (154 mg, 107 μL, 2.0 equiv, 2.89 mmol), sodium bicarbonate (607 mg, 281 μL, 5.0 equiv, 7.22 mmol) and HATU (824 mg, 1.5 equiv, 2.17 mmol) in DMF (1 mL) was stirred at room temperature. The reaction was quenched with water and extracted with EA. The organic layer was washed with brine, dried over Na₂SO₄ and evaporated. This resulted in compound 395-8 (400 mg, 723 μmol, 50% yield) a yellow solid.

m/z (ES+) [M+H]+=553.4

Step 8: To a stirred solution of compound 395-8 (100 mg, 1.0 equiv, 181 μmol) in DCM (1 mL) was added pyridine (18 g, 18 mL, 0.22 mol) and Tf₂O (20 g, 9 mL, 0.05 mol) at 0° C. The resulting mixture was stirred at 0° C. for 1 hour. The resulting mixture was dropwise to NaHCO₃ solution at 0° C. The reaction was quenched with water and extracted with EA. The organic layer was washed with brine, dried over Na₂SO₄ and evaporated. This resulted in compound 395-9 (100 mg, 187 μmol, 103%) as a yellow solid.

m/z (ES+) [M+H]+=535.15

Compound 395-9 was transformed into the final product 395 using the analogous procedures from Example 392.

m/z (ES+) [M+H]+=575.25

¹H NMR (400 MHZ, Chloroform-d) 8.66-8.60 (m, 1H), 8.10 (s, 1H), 7.92 (s, 1H), 7.86 (d, J=3.1 Hz, 2H), 7.79-7.68 (m, 3H), 7.49 (d, J=8.0 Hz, 1H), 7.43 (d, J=8.3 Hz, 1H), 7.26 (s, 1H), 7.16 (d, J=8.3 Hz, 1H), 5.24 (s, 1H), 4.21 (t, J=51.9 Hz, 5H), 3.44 (s, 3H), 2.31 (s, 3H), 1.57 (tt, J=8.0, 4.4 Hz, 2H), 1.20-1.11 (m, 2H), 0.98 (dq, J=7.5, 4.1 Hz, 2H).

### Example 396

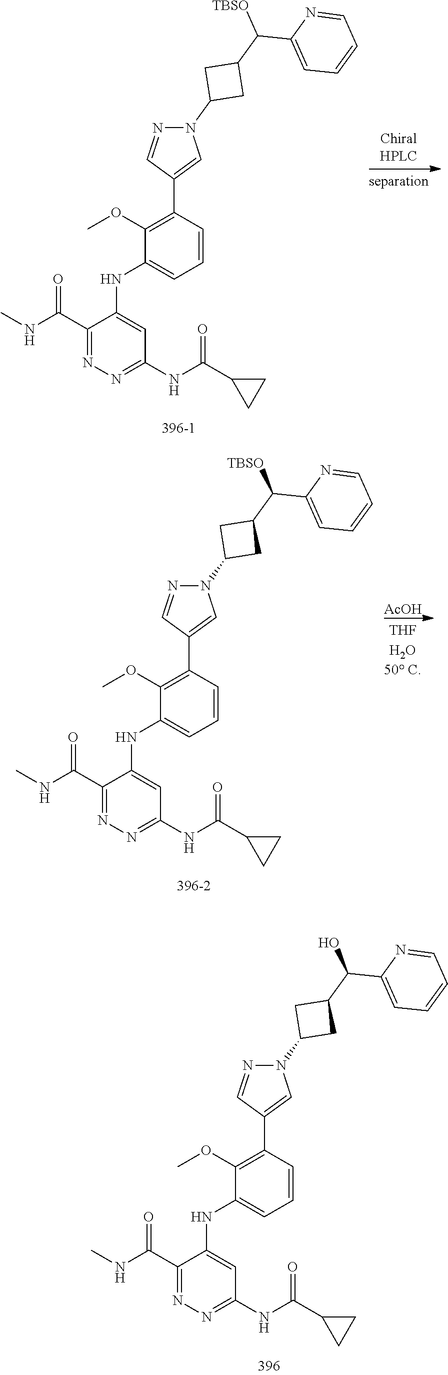

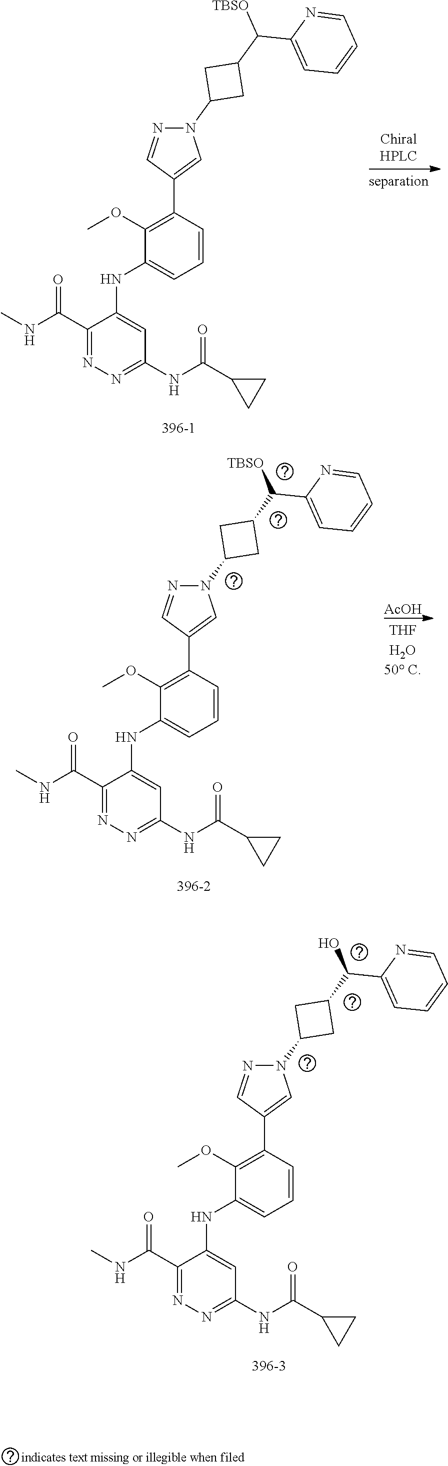

Compound 396-1 was prepared using analogous procedures in Example 346 except that 2-((3-(4-bromo-1H-pyrazol-1-yl)cyclobutyl)((tert-butyldimethylsilyl)oxy)methyl)pyridine was used in place of compound 346-5.

The four diastereomers of 396-1 were separated under the following chiral HPLC conditions: Lux 5 μm Cellulose-2, 3*15 cm, 5 um; Mobile Phase A: Hex (0.3% IPAmine)-HPLC, Mobile Phase B: EtOH-HPLC; Flow rate: 20 mL/min; Gradient: 40% B to 40% B in 20 min; Wave Length: 220/254 nm; RT1 (min): 11.09; RT2 (min): 14.48; Sample Solvent: EtOH-HPLC; Injection Volume: 1 mL

Compound 396-2 was isolated as an off-white solid and deprotected under the following conditions: A 8 mL vial was charged with compound 396-2 (49 mg, 1.0 equiv, 72 μmol), excess AcOH (1.6 g, 1.5 mL, 26 mmol) and a stir bar. THF (1 mL) and water (1 mL) was added, and the solution was stirred at 50° C. for 24 hours.

The reaction was concentrated, resuspended in DMSO, filtered and the purified by reverse phase chromatography (column: Xselect CSH C18 OBD, gradient: Water (0.1% FA)/MeCN). The product containing fractions were concentrated to provide compound 396 as a single cis-cyclobbutyl diastereomer (absolute stereochemistry arbitrarily assigned) (15.9 mg, 28.0 μmol, 39%) as a white amorphous solid.

m/z (ES+) [M+H]+=569.3

¹H NMR (400 MHZ, DMSO-d6) 11.31 (s, 1H), 10.99 (s, 1H), 9.17 (q, J=4.7 Hz, 1H), 8.49 (dd, J=4.9, 1.8 Hz, 1H), 8.25 (s, 1H), 8.16 (s, 1H), 7.96 (s, 1H), 7.78 (td, J=7.7, 1.8 Hz, 1H), 7.49 (d, J=7.5 Hz, 1H), 7.49-7.43 (m, 1H), 7.33-7.21 (m, 2H), 7.20 (t, J=7.9 Hz, 1H), 5.56 (s, 1H), 4.78-4.65 (m, 1H), 4.65-4.59 (m, 1H), 3.60 (s, 3H), 2.87 (d, J=4.7 Hz, 3H), 2.52 (s, 1H), 2.49-2.41 (m, 2H), 2.37-2.22 (m, 2H), 2.09 (p, J=6.3 Hz, 1H), 0.88 [symbol] C 0.79 (m, 4H).

### Example 397

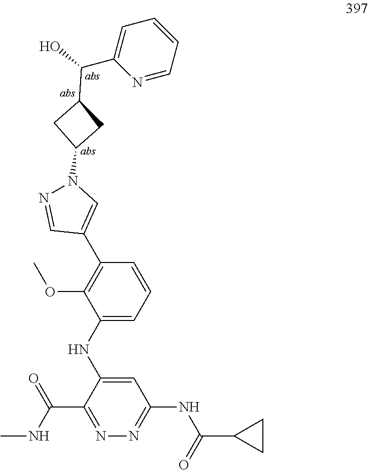 [SMILES: CNC(=O)c1nnc(NC(=O)C2CC2)cc1Nc1cccc(-c2cnn([C@H]3C[C@H]([C@H](O)c4ccccn4)C3)c2)c1OC]

Compound 397 was isolated as a single cis-cyclobbutyl diastereomer (absolute stereochemistry arbitrarily assigned) using the procedures in example 396.

m/z (ES+) [M+H]+=569.3

¹H NMR (400 MHZ, DMSO-d6) 11.31 (s, 1H), 10.96 (s, 1H), 9.16 (q, J=4.7 Hz, 1H), 8.50 (ddd, J=4.9, 1.9, 0.9 Hz, 1H), 8.29-8.25 (m, 1H), 8.15 (s, 1H), 7.96 (s, 1H), 7.80 (td, J=7.7, 1.8 Hz, 1H), 7.54 (dt, J=8.0, 1.2 Hz, 1H), 7.45 (dd, J=7.8, 1.6 Hz, 1H), 7.32-7.22 (m, 2H), 7.19 (t, J=7.9 Hz, 1H), 5.66 (d, J=5.0 Hz, 1H), 4.99 (p, J=7.8 Hz, 1H), 4.73 (t, J=5.5 Hz, 1H), 3.58 (s, 3H), 2.87 (d, J=4.7 Hz, 3H), 2.77 (dq, J=9.7, 4.9 Hz, 1H), 2.61-2.51 (m, 2H), 2.44 (ddd, J=19.4, 10.4, 6.4 Hz, 2H), 2.08 (tt, J=6.9, 5.3 Hz, 1H), 0.88-0.79 (m, 4H).

### Example 398

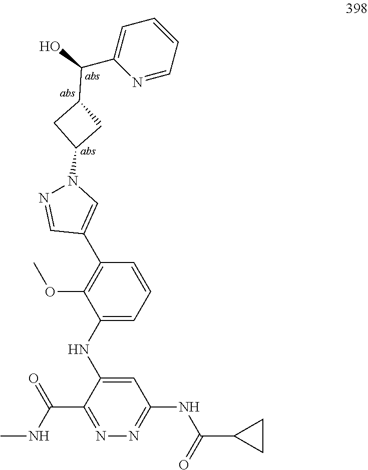 [SMILES: CNC(=O)c1nnc(NC(=O)C2CC2)cc1Nc1cccc(-c2cnn([C@H]3C[C@@H]([C@@H](O)c4ccccn4)C3)c2)c1OC]

Compound 398 was isolated as a single trans-cyclobbutyl diastereomer (absolute stereochemistry arbitrarily assigned) using the procedures in example 396.

m/z (ES+) [M+H]+=569.3

¹H NMR (400 MHZ, DMSO-d6) 11.31 (s, 1H), 10.97 (s, 1H), 9.16 (q, J=4.8 Hz, 1H), 8.50 (ddd, J=4.8, 1.8, 0.9 Hz, 1H), 8.27 (s, 1H), 8.15 (s, 1H), 7.97 (s, 1H), 7.80 (td, J=7.7, 1.8 Hz, 1H), 7.54 (dd, J=7.9, 1.2 Hz, 1H), 7.45 (dd, J=7.8, 1.6 Hz, 1H), 7.32-7.22 (m, 2H), 7.19 (t, J=7.9 Hz, 1H), 5.69-5.64 (m, 1H), 4.99 (p, J=7.8 Hz, 1H), 4.76-4.69 (m, 1H), 3.58 (s, 3H), 2.87 (d, J=4.8 Hz, 3H), 2.77 (dq, J=9.6, 4.8 Hz, 1H), 2.61-2.51 (m, 2H), 2.44 (ddd, J=19.3, 10.4, 6.3 Hz, 2H), 2.14-2.03 (m, 1H), 0.88-0.79 (m, 4H).

### Example 399

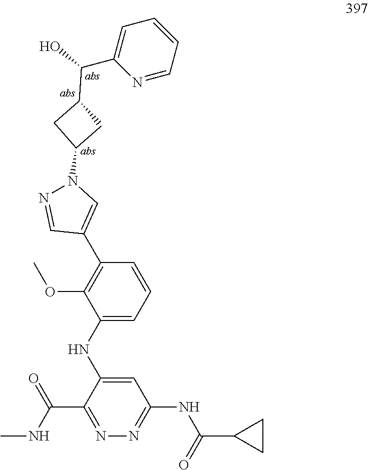 [SMILES: CNC(=O)c1nnc(NC(=O)C2CC2)cc1Nc1cccc(-c2cnn([C@H]3C[C@@H]([C@H](O)c4ccccn4)C3)c2)c1OC]

Compound 399 was isolated as a single trans-cyclobbutyl diastereomer (absolute stereochemistry arbitrarily assigned) using the procedures in example 396.

m/z (ES+) [M+H]+=569.3

¹H NMR (400 MHZ, DMSO-d6) 11.31 (s, 1H), 10.98 (s, 1H), 9.17 (q, J=4.8 Hz, 1H), 8.52-8.46 (m, 1H), 8.25 (s, 1H), 8.16 (s, 1H), 7.96 (s, 1H), 7.78 (td, J=7.7, 1.8 Hz, 1H), 7.52-7.44 (m, 2H), 7.32-7.16 (m, 3H), 5.56 (d, J=5.1 Hz, 1H), 4.78-4.65 (m, 1H), 4.62 (t, J=4.6 Hz, 1H), 3.60 (s, 3H), 2.87 (d, J=4.8 Hz, 3H), 2.52 (s, 1H), 2.50-2.43 (m, 2H), 2.37-2.22 (m, 2H), 2.08 (h, J=5.8, 5.4 Hz, 1H), 0.88-0.79 (m, 4H).

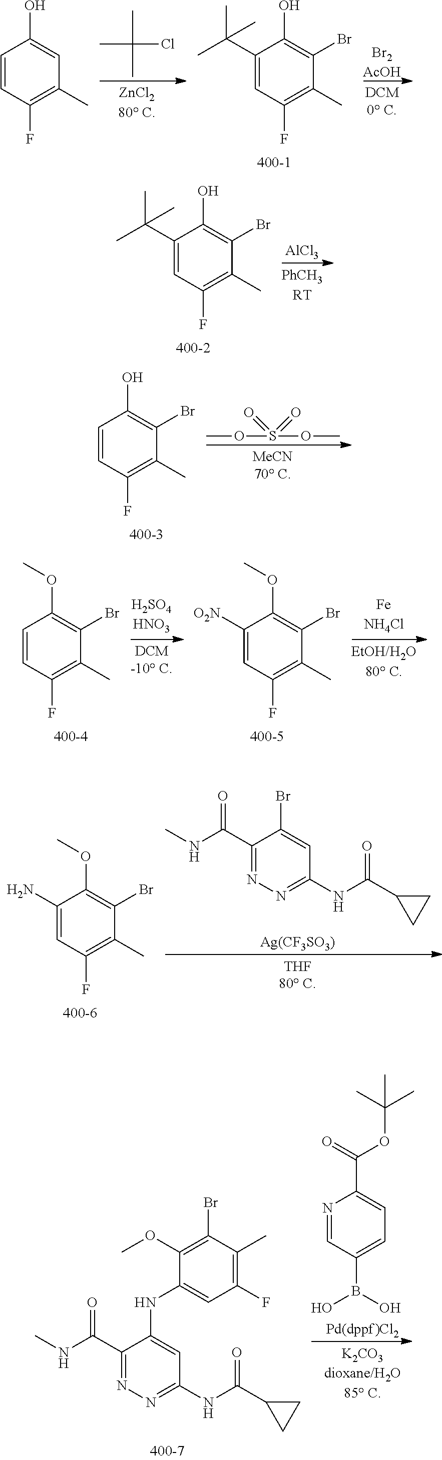

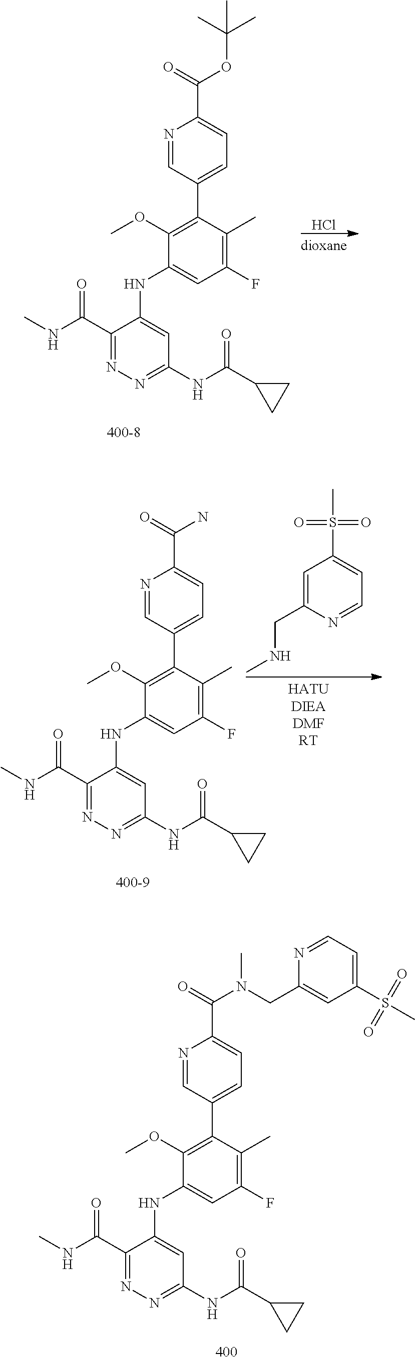

Step 1: A round bottomed flask was charged with 4-fluoro-3-methylphenol (10 g, 1.0 equiv, 79 mmol), 2-chloro-2-methylpropane (15 g, 2.0 equiv, 0.16 mol), ZnCl₂ (40 mL) was added, and the solution was stirred at 80° C. for 3 hour until the reaction was determined to be complete by LC/MS analysis. The resulting mixture was extracted with EtOAc. The combined organic layers were washed with water and then washed with brine, dried over anhydrous Na₂SO₄. After filtration, the filtrate was concentrated under reduced pressure. The residue was purified by flash silica gel column chromatography to afford compound 400-1 (14 g, 77 mmol, 97% yield).

m/z (ES+) [M+H]−=180.9

Step 2: A round bottomed flask was charged with compound 400-1 (5 g, 1 equiv, 0.03 mol), DCM (50 mL) and a stirbar. Bromine (9 g, 3 mL, 2 equiv, 0.05 mol), AcOH (0.8 g, 0.8 mL, 0.5 equiv, 0.01 mol) were added, and the solution was stirred at 0° C. for 12 hours. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatography to afford compound 400-2 (5.2 g, 20 mmol, 70% yield).

m/z (ES+) [M+H]−=259.1

Step 3: A round bottomed flask was charged with compound 400-2 (5 g, 1 equiv, 0.02 mol), toluene (60 mL) and a stirbar, after which aluminum trichloride (3 g, 1.2 equiv, 0.02 mol) was added, and the solution was stirred at 25° C. for 2 h until the reaction was determined to be complete by LC/MS analysis. The resulting mixture was extracted with EtOAc. The combined organic layers were washed with water and then washed with brine, dried over anhydrous Na₂SO₄. After filtration, the filtrate was concentrated under reduced pressure. The residue was purified by silica gel column chromatography to compound 400-3 (2.5 g, 12 mmol, 60% yield).

m/z (ES+) [M+H]−=203.2

Step 4: A round bottomed flask was charged with compound 400-3 (2.4 g, 1.0 equiv, 12 mmol), K₂CO₃ (3.2 g, 2.0 equiv, 23 mmol), MeCN (0.5 mL) and a stirbar, after which dimethyl sulfate (2.2 g, 1.5 equiv, 18 mmol) was added, and the solution was stirred at 80° C. for 1 hour until the reaction was determined to be complete by LC/MS analysis. The resulting mixture was extracted with EtOAc. The combined organic layers were washed with water and then washed with brine, and dried over anhydrous Na₂SO₄. After filtration, the filtrate was concentrated under reduced pressure. The residue was purified by silica gel column chromatography to afford compound 400-4 (3 g, 0.01 mol, 100% yield) as a crude product. The crude product was used in next step directly without further workup.

m/z (ES+) [M+H]+=219.1

Step 5: A round bottomed flask was charged compound 400-4 (2.5 g, 1.0 equiv, 11 mmol), DCM (5 mL) and a stirbar. H₂SO₄:HNO₃ (1:1 v/v, 5 mL) was added, and the solution was stirred at −10° C. for 5 min until the reaction was determined to be complete by LC/MS analysis. The resulting mixture was extracted with EtOAc. The combined organic layers were washed with water and then washed with brine, and dried over anhydrous Na₂SO₄. After filtration, the filtrate was concentrated under reduced pressure to afford compound 400-5 (2.5 g, 9.5 mmol, 83% yield) as a crude product. The crude product was used in next step directly without further workup.

m/z (ES+) [M+H]+=264.0

Step 6: A round bottomed flask was charged with compound 400-5 (2.5 g, 1 equiv, 9.5 mmol), ammonium chloride (2.5 g, 5 equiv, 47 mmol), EtOH (20 mL), H₂O (4 mL) and a stirbar. Iron (2.6 g, 5.0 equiv, 47 mmol) was added, and the solution was stirred at 80° C. for 5 hour. The resulting mixture was filtered, the filter cake was washed with DCM. The filtrate was extracted with DCM. The combined organic layers were washed with water and then washed with brine, dried over anhydrous Na₂SO₄. After filtration, the filtrate was concentrated under reduced pressure. The residue was purified by to afford compound 400-6 (2 g, 9 mmol, 90%).

m/z (ES+) [M+H]+=233.9

Step 7: A round bottomed flask was charged with compound 400-6 (2 g, 1 equiv, 9 mmol), 4-bromo-6-(cyclopropanecarboxamido)-N-methylpyridazine-3-carboxamide (3 g, 1.2 equiv, 0.01 mol), THF (20 mL) and a stirbar. Ag(OTf) (4 g, 2 equiv, 0.02 mol) was added, and the solution was stirred at 80° C. for 2 hours. The resulting mixture was filtered, the filter cake was washed with DCM. The filtrate was extracted with DCM. The combined organic layers were washed with water and then washed with brine, dried over anhydrous Na₂SO₄. After filtration, the filtrate was concentrated under reduced pressure. The residue was purified by silica gel column chromatography to afford compound 400-7 (2.6 g, 5.7 mmol, 70% yield).

m/z (ES+) [M+H]+=452.0

Step 8: A round bottomed flask was charged with compound 400-7 (2 g, 1 equiv, 4 mmol), (6-(tert-butoxycarbonyl)pyridin-3-yl) boronic acid (1 g, 1.2 equiv, 5 mmol), K₂CO₃ (1 g, 2 equiv, 9 mmol), H₂O (0.2 mL), 1,4-dioxane (1 mL) and a stirbar. PdCl₂(dppf) (0.6 g, 0.2 Eq, 0.9 mmol) was added, and the solution was stirred at 85° C. for 2 hour until the reaction was determined to be complete by LC/MS analysis. The resulting mixture was extracted with EtOAc. The combined organic layers were washed with water and then washed with brine, and dried over anhydrous Na₂SO₄. After filtration, the filtrate was concentrated under reduced pressure. The residue was purified by silica gel column chromatography to afford compound 400-8 (1.2 g, 2.2 mmol, 50% yield).

m/z (ES+) [M+H]+=551.2

Step 9: A round bottomed flask was charged with compound 400-8 (300 mg, 1.0 equiv, 545 μmol), DCM (3 mL) and a stirbar. HCl/dioxane 3 mL was added, and the solution was stirred at 25° C. for 4 hours until the reaction was determined to be complete by LC/MS analysis. The resulting mixture was concentrated under reduced pressure to afford compound 400-9 (200 mg, 404 μmol, 74% yield) as a crude product. The crude product was used in next step directly without further workup.

m/z (ES+) [M+H]+=451.9

Step 10: A round bottomed flask was charged with compound 400-9 (150 mg, 1.0 equiv, 303 μmol), N-methyl-1-(4-(methylsulfonyl)pyridin-2-yl) methanamine (72.9 mg, 1.2 equiv, 364 μmol), HATU (231 mg, 2 Eq, 607 μmol), DMF (5 mL) and a stirbar. DIEA (78.4 mg, 106 μL, 2.0 equiv, 607 μmol) was added, and the solution was stirred at 25° C. for 3 hours until the reaction was determined to be complete by LC/MS analysis. The resulting mixture was extracted with EtOAc. The combined organic layers were washed with water and then washed with brine, dried over anhydrous Na₂SO₄. After filtration, the filtrate was concentrated under reduced pressure. The crude product was purified by Prep-HPLC to afford compound 400 (78.1 mg, 114 μmol, 38% yield) as a white amorphous solid.

m/z (ES+) [M+H]+=677.1

¹H NMR (400 MHZ, DMSO-d6) 11.38 (d, J=5.3 Hz, 1H), 10.97 (d, J=12.1 Hz, 1H), 9.22-9.15 (m, 1H), 8.89 (dd, J=27.8, 5.0 Hz, 1H), 8.53 (dd, J=62.1, 2.1 Hz, 1H), 8.21 (d, J=10.6 Hz, 1H), 8.02-7.71 (m, 4H), 7.42 (dd, J=12.8, 10.7 Hz, 1H), 4.97 (d, J=6.6 Hz, 2H), 3.37 (s, 1H), 3.32 (s, 2H), 3.26 (d, J=32.9 Hz, 3H), 3.12 (d, J=28.2 Hz, 3H), 2.84 (dd, J=4.8, 3.3 Hz, 3H), 2.18-2.06 (m, 1H), 1.98 (dd, J=26.1, 2.3 Hz, 3H), 0.86 (dd, J=8.0, 4.9 Hz, 4H).

### Example 401

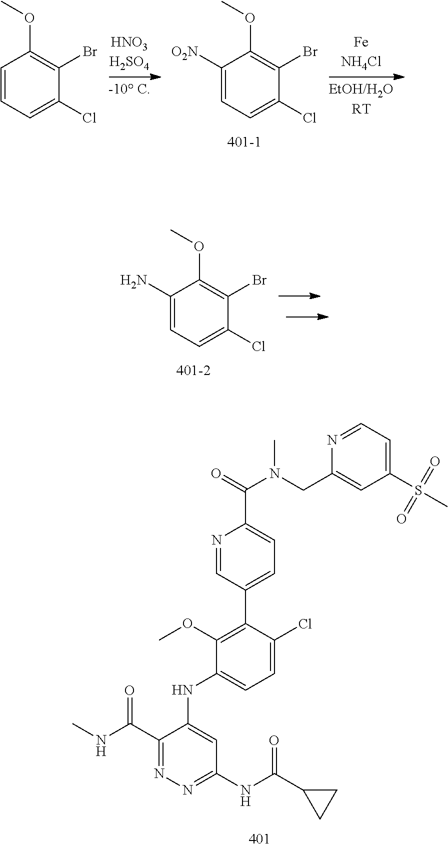

Step 1: A round bottomed flask was charged with 2-bromo-1-chloro-3-methoxybenzene (1.1 g, 1 equiv, 5.0 mmol), nitric acid (2.2 g, 1.8 mL, 7 equiv, 35 mmol) and sulfuric acid (fuming 60%) (6.2 g, 3.2 mL, 7 equiv, 35 mmol), and the solution was stirred at −10° C. for 5 min. The resulting mixture was concentrated under reduced pressure. The crude product was purified by Prep-HPLC to afford compound 401-1 (305 mg, 1.14 mmol, 23% yield) as an off-white amorphous solid.

m/z (ES+) [M+H]+=266.0

Step 2: A round bottomed flask was charged with compound 401-1 (280 mg, 1.0 equiv, 1.05 mmol), EtOH (10 mL), H₂O (2.0 mL) and a stirbar. Ammonium chloride (112 mg, 77.6 μL, 2.0 equiv, 2.10 mmol), iron (176 mg, 22.4 μL, 3.0 equiv, 3.15 mmol) was added, and the solution was stirred at 20° C. for 2 hours.

The resulting mixture was extracted with EtOAc. The combined organic layers were washed with brine and then washed with water, dried over anhydrous Na₂SO₄. After filtration, the filtrate was concentrated under reduced pressure. The resulting crude material was purified by silica gel chromatography. Concentration in vacuo resulted compound 401-2 (300 mg, 0.89 mmol, 85% yield) as an off white amorphous solid

m/z (ES+) [M+H]+=236.0

Compound 401-2 was converted to compound 401 using the analogous procedures from Example 400.

m/z (ES+) [M+H]+=479.2

¹H NMR (400 MHZ, DMSO-d6) 11.40 (d, J=5.5 Hz, 1H), 10.96 (d, J=12.5 Hz, 1H), 9.27-9.15 (m, 1H), 8.90 (dd, J=26.1, 5.0 Hz, 1H), 8.57 (dd, J=60.4, 2.2 Hz, 1H), 8.17 (d, J=11.3 Hz, 1H), 8.06-7.72 (m, 4H), 7.62-7.43 (m, 2H), 4.96 (d, J=5.1 Hz, 2H), 3.38 (d, J=1.6 Hz, 3H), 3.31 (d, J=8.3 Hz, 3H), 3.11 (d, J=30.8 Hz, 3H), 2.87-2.77 (m, 3H), 2.10 (d, J=6.1 Hz, 1H), 0.85 (h, J=4.2, 3.5 Hz, 4H).

### Example 404

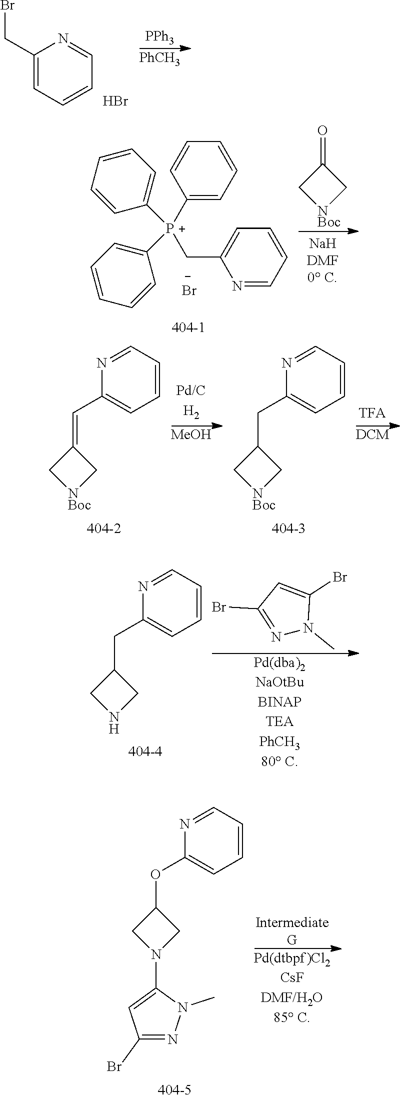

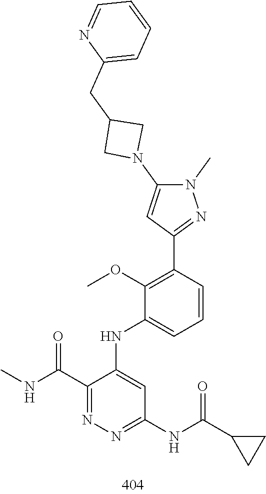 [SMILES: CNC(=O)c1nnc(NC(=O)C2CC2)cc1Nc1cccc(-c2cc(N3CC(Cc4ccccn4)C3)n(C)n2)c1OC]

Step 1: A round bottomed flask was charged with 2-(bromomethyl)pyridine hydrobromide (6 g, 1.0 equiv, 0.02 mol), and triphenylphosphine (8 g, 7 mL, 1.3 equiv, 0.03 mol). Toluene (60 mL) was added, and the solution was stirred at 115° C. for 12 hours. The mixture was filtered and the filtrate cake was washed with EtOH to provide 404-1 (7 g, 0.02 mol, 70% yield) as a brown solid.

m/z (ES+) [M]+=354.3

Step 2: To a cold solution of compound 404-1 (3 g, 1 equiv, 7 mmol) in DMF (20 mL) was added NaH (0.4 g, 60% Wt, 1.5 equiv, 0.01 mol) at 0° C. after which the resulting mixture was stirred at 0° C. for 15 min. A solution of tert-butyl 3-oxoazetidine-1-carboxylate was then added (1 g, 1.2 equiv, 8 mmol) in DMF (10 mL) was added and the reaction was stirred at 65° C. for 12 hours. After the reaction was complete, the reaction was quenched with a saturated NH₄Cl solution and extracted with EtOAc. The organic layer were washed with water and brine, then dried over anhydrous Na₂SO₄, filtered and evaporated. This residue was purified by column chromatography on silica gel (petroleum ether/ethyl acetate=5:1) to give compound 404-2 (800 mg, 3.25 mmol, 50% yield) as a white solid.

m/z (ES+) [M+H]+=247.4

Step 3: A round bottomed flask was charged compound 404-2 (750 mg, 1.0 equiv, 3.04 mmol) and Pd/C(32.4 mg, 0.1 equiv, 304 μmol). MeOH (10 mL) was added, and the solution was stirred at 25° C. for 1 hour under H₂. The mixture was filtered and the filtrate cake was washed with methanol. The filtrate was evaporated to compound 404-3 (650 mg, 2.62 mmol, 86% yield) as a yellow solid used in next step directly.

m/z (ES+) [M+H]+=249.1

Step 4: A round bottomed flask was charged with compound 404-3 (650 mg, 1.0 equiv, 2.62 mmol) in DCM (6 mL). TFA (3 mL) was then added, and the solution was stirred at 25° C. for 30 min. The mixture was evaporated to give compound 404-4 (900 mg, 2.4 mmol, 93% yield) as a yellow oil used in next step directly.

m/z (ES+) [M+H]+=149.1

Step 5: A round bottomed flask was charged with compound 404-4 (800 mg, 1.0 equiv, 5.40 mmol), 3,5-dibromo-1-methyl-1H-pyrazole (1.94 g, 1.5 equiv, 8.10 mmol), TEA (1.64 g, 2.26 mL, 3.0 equiv, 16.2 mmol), NaOtBu (1.56 g, 1.75 mL, 3.0 equiv, 16.2 mmol), BINAP (672 mg, 0.2 equiv, 1.08 mmol), and Pd₂(dba)₃ (494 mg, 0.1 equiv, 540 μmol). Toluene (10 mL) was added, and the solution was stirred at 80° C. for 24 hours under N₂. The reaction was quenched with water and extracted with EA. The organic layer was washed with brine, dried over Na₂SO₄ and evaporated. The crude product was then purified using C18 flash chromatography (gradient: MeCN/water). This resulted in compound 404-5 (100 mg, 326 μmol, 6% yield) as a yellow solid.

m/z (ES+) [M+H]+=307.2

Step 6: A round bottomed flask was charged with compound 404-5 (95 mg, 1.0 equiv, 0.31 mmol), Intermediate G (0.14 g, 1.0 equiv, 0.31 mmol), CsF (94 mg, 2.0 equiv, 0.62 mmol) and 1,1′-bis(di-t-butylphosphino) ferrocene palladium dichloride (40 mg, 0.2 equiv, 62 μmol). DMF (5 mL) and water (1 mL) were added, and the solution was stirred at 80° C. for 2 hours under N₂. The resulted solution was purified using PREP-HPLC (column: Xselect CSH C18 OBD, gradient: Water (0.1% FA)/MeCN). Lyophilization yielded compound 404 (20.2 mg, 35.6 μmol, 12% yield) as an off-white amorphous solid.

m/z (ES+) [M+H]+=568.2

¹H NMR (400 MHZ, Methanol-d4) 8.90 (d, J=6.1 Hz, 1H), 8.56 (s, 1H), 8.50 (t, J=7.8 Hz, 1H), 8.24 (s, 1H), 8.07 (d, J=8.0 Hz, 1H), 7.95 (t, J=6.8 Hz, 1H), 7.56 (dd, J=7.8, 1.6 Hz, 1H), 7.46 (dd, J=8.0, 1.6 Hz, 1H), 7.23 (t, J=7.9 Hz, 1H), 6.09 (s, 1H), 5.05 (dd, J=13.4, 7.7 Hz, 1H), 4.83 (dd, J=13.3, 5.8 Hz, 1H), 3.78 (dd, J=18.3, 8.1 Hz, 1H), 3.66 (d, J=2.2 Hz, 6H), 3.56-3.36 (m, 3H), 3.01 (s, 3H), 1.95 (tt, J=7.7, 4.6 Hz, 1H), 1.03-0.88 (m, 4H).

### Example 408

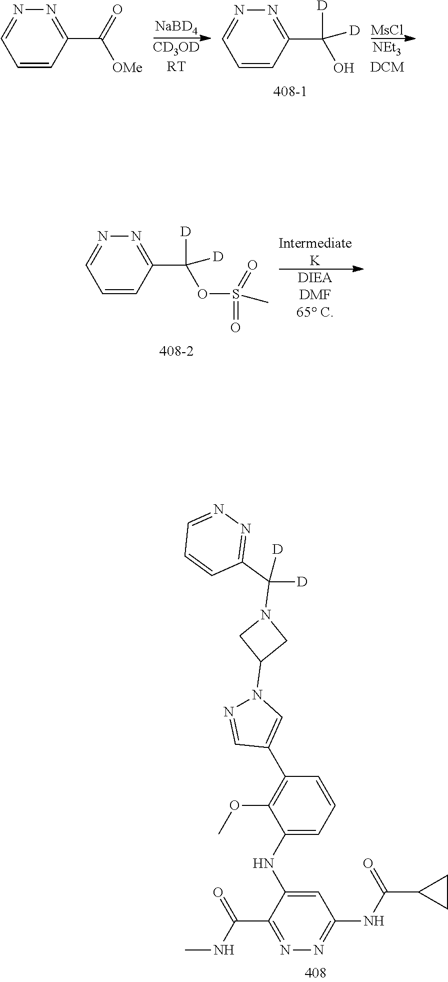

Step 1: To a stirred suspension of methyl pyridazine-3-carboxylate (2.646 g, 1.0 equiv, 19.16 mmol) in methanol-d4 (3 mL) at RT was added sodium tetrahydroborate-d4 (4.009 g, 3.733 mL, 5.0 equiv, 95.78 mmol). The mixture turned temporarily yellow with stirring and exhibited a mild exotherm. The reaction was stirred for 50 minutes at 40° C. until the reaction was determined to be complete by LC/MS analysis. The reaction was then treated with 20 mL of H₂O and 20 mL of brine and then extracted with 4:1 DCM/iPrOH (3×200 mL), followed by 5% MeOH/DCM (6×100 mL). The combined organic layers were dried over sodium sulfate, filtered, concentrated down, and re-filtered with DCM to remove insoluble inorganics. The filtrate was concentrated down and dried under vacuum to compound 408-1 (1.455 g, 12.98 mmol, 68% yield) as a cloudy, thick yellow oil that was used directly in the next step.

Step 2: To a stirred mixture of compound 408-1 (410 mg, 1.0 equiv, 3.66 mmol) and triethylamine (1.11 g, 1.53 mL, 3.0 equiv, 11.0 mmol) in DCM (8 mL) at RT was added one half portion of methanesulfonyl chloride (838 mg, 566 μL, 2.0 equiv 7.31 mmol). After 1 hour, the second half of methanesulfonyl chloride was added. The reaction was stirred a total of 3 hours until it was determined to be complete by TLC analysis after which it was diluted with DCM (30 mL) and washed with brine (10 mL). The aqueous layer was extracted with fresh DCM (30 mL). The combined organic layers were dried over sodium sulfate, filtered, concentrated down, and dried to provide compound 408-2 (586.7 mg, 3.084 mmol, 84% yield) as a dark amber, thick oil that was used directly in the next step.

Step 3: To a slurry of Intermediate K (300 mg, 1.0 equiv, 649 μmol) and diisopropylethylamine (252 mg, 334 μL, 3.0 equiv, 1.95 mmol) in DMF (2 mL) in a sealed tube was added a solution of compound 408-2 (160 mg, 1.3 equiv, 843 μmol) dissolved in DMF (0.35 mL). The sealed tube was stirred at 65° C. for 2 hours. Additional compound 408-2 (160 mg, 1.3 equiv, 843 μmol) was added after about 1 hour of stirring. The reaction was cooled to RT. and treated with water (15 mL), followed by the addition of aqueous 2N NaOH (2 mL). The tan precipitate that formed was isolated by filtration, dried, dissolved in DCM, and purified by silica gel chromatography (gradient: DCM/10:90:0.5 MeOH/DCM/NH₄OH) to provide compound 408 (114.1 mg, 0.20 mmol, 31% yield) as a tan, foamy solid.

LC-MS (ESI+) m/z: 557.4 [M+H]+

¹H NMR (500 MHz, DMSO) δ 11.29 (s, 1H), 10.96 (s, 1H), 9.19-9.10 (m, 2H), 8.37 (d, J=0.7 Hz, 1H), 8.13 (s, 1H), 8.02 (d, J=0.8 Hz, 1H), 7.72-7.64 (m, 2H), 7.47 (dd, J=7.8, 1.6 Hz, 1H), 7.29 (dd, J=8.0, 1.5 Hz, 1H), 7.19 (t, J=7.9 Hz, 1H), 5.11 (p, J=6.9 Hz, 1H), 3.80 (td, J=7.1, 1.7 Hz, 2H), 3.63 (td, J=6.7, 1.6 Hz, 2H), 3.58 (s, 3H), 2.86 (d, J=4.8 Hz, 3H), 2.07 (tt, J=7.0, 5.3 Hz, 1H), 0.81 (dt, J=7.9, 2.3 Hz, 4H).

### Example 409

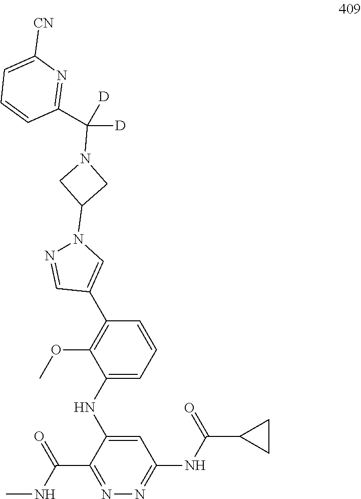 [SMILES: [2H]C([2H])(c1cccc(C#N)n1)N1CC(n2cc(-c3cccc(Nc4cc(NC(=O)C5CC5)nnc4C(=O)NC)c3OC)cn2)C1]

Example 409 is prepared in using analogous procedures form Example 408 except that methyl 6-cyanopicolinate was used in place of methyl pyridazine-3-carboxylate. LC/MS (ES+) m/z=581.2 [M+H]+

### Example A1—Surface Plasmon Resonance Binding Assay

Selected compounds of the present disclosure were tested in a Surface Plasmon Resonance (SPR) Assay to measure their binding kinetics and affinity for proteins of interest.

| Materials | Vendor | Catalog number |
|---|---|---|
| 1M HEPES pH 7.5 | Boston BioProducts | BBH-75 |
| 5M NaCl | Sigma-Aldrich | S6546-1L |
| 10% Tween | Fisher Scientific | 50843255 |
| Dithiothreitol powder | Fisher Scientific | ICN19482110 |
| JAK2 JH1* or JH2** | Biortus | |
| Domain “WT” | | |
| Biotin CAPture Kit, Series S | Global Life Sciences | 28920234 |
| | Solutions | |
| Biotin | Sigma-Aldrich | B4501 |

*JAK2 JH1 WT (T842-G1132) N-His60-avi-TEV
**JAK2 JH2 WT (V536-D812) [W659A, W777A, F794H solubilizing mutations] C-thrombin-avi-His6

| Instrument | Vendor |
|---|---|
| Biacore 8K SPR system | Cytiva (formerly GE Healthcare) |
| Bravo liquid handler | Agilent |

Prepare buffer: 50 mM HEPES pH 7.5, 150 mM NaCl, 0.05% (v/v) Tween, and 10% (v/v) glycerol were combined and pH was adjusted to 7.5, then filtered. 5 mM DTT, was added, and buffer without DMSO was reserved for compound resuspension. 2% (v/v) DMSO was added for assay buffer, and separately 1% DMSO was used to make buffer for resuspending compounds.

Prepare compounds: Using a Bravo liquid handler, 99 μL of 1% (v/v) DMSO assay buffer was added to 1 μL compounds in DMSO.

Run 8K Method: Biotin CAPture (CAP) chip surface was regenerated prior to run by: Inserting CAP chip into Biacore 8K, prime machine with assay buffer with 2% (v/v) DMSO; Setting instrument temperature to 20° C.; Conditioning surface with NaOH/GdnHCl reagents from CAP chip kit (60 seconds, 10 μL/min); and Capturing oligo-Streptavidin (SA) reagent from CAP kit (600 seconds, 1 μL/min), expected capture ˜2500 RU. JAK2 JH1 or JH2 protein was immobilized by preparing a sample of 0.5 uM JH1 or 0.4 uM JH2 protein diluted in assay buffer without DMSO and supplemented with 10 uM of a selected compound of the invention as a stabilizer (370 seconds, 5 uL/min), expected capture ˜1000 RU. Remaining biotin sites on SA were blocked with 1 uM biotin (30 seconds, 20 uL/min), then equilibrated for 1800 seconds at 100 uL/min. Compounds being assayed were run in single cycle kinetics: 4 doses with a top concentration of 5 uM (JH1) or 500 nM (JH2), dilution factor of 3, at 20° C., for a contact time of 60 seconds, and a dissociation time of 300 seconds (JH1) or 1200 seconds (JH2), at a flow rate of 30 uL/min. The surface was completely regenerated after each compound is run, and JH1 or JH2 protein was immobilized fresh prior to each new compound. Data were fitted kinetically with 1:1 binding model using Biacore Insight Evaluation Software.

Results of the SPR Binding Assay using JAK2 JH1 or JH2 domain proteins are presented in Table 1. Compounds having a dissociation constant (K_{D}) greater than 10 pM, but less than or equal to 10 nM are represented as “A”; compounds having a K_{D} greater than 10 nM but less than or equal to 50 nM are represented as “B”; compounds having a K_{D} greater than 50 nM but less than or equal to 500 nM are represented as “C”; compounds having a K_{D} greater than 500 nM but less than or equal to 5 UM are represented as “D”; and compounds having a K_{D} greater than 5 uM but less than or equal to 100 UM are represented as “E”.

### Example A2—Cellular TF1 STAT3 and STAT5 Assays

Selected compounds of the present disclosure were tested in a Cellular TF1 STAT3 or STAT5 assay, to measure the degree of JAK2-mediated STAT3 or STAT5 phosphorylation, respectively. TF1 cells (immortalized erythroleukemia line) expressing either wildtype or V617F mutant JAK2 with a luciferase reporter were used.

| Materials | Vendor | Catalog number |
|---|---|---|
| RPMI 1640 Medium | | |
| Fetal Bovine Serum (FBS) | | |
| 384 well white clear bottom plate, | Corning | 3762 |
| tissue culture treated | | |
| ONE-Glo Luciferase Assay System | Promega | E6130 |

| Instrument | Vendor |
|---|---|
| Vi-CELL XR | Beckman Coulter |
| Echo 555 LIQUID HANDLER | Labcyte |
| Biomek Liquid Handler | Beckman |
| En Vision Multimode Plate Reader | Perkin Elmer |
| Cell line | Growth Media |
| TF1 JAK2 V617F STAT3 reporter | RPMI 1640 + 10% FBS |
| TF1 JAK2 V617F STAT5 reporter | RPMI 1640 + 10% FBS |

Cell culture: Cells were put into antibiotic selection (1 ug/ml puro+500 ug/ml Geneticin) when first thawed then maintained in growth media without antibiotics. Cells were maintained in exponential growth phase, between 0.02-1 E6 cells/ml.

Compound preparation: Compounds were screened as 10-point dose response curves 1:3 dilutions. DMSO is used as the vehicle control. All wells were normalized to the same final DMSO concentration. Compounds were pre-spotted onto 384 well plates using an Echo acoustic dispenser, 50 nL of 1000× (10 mM top concentration) DMSO stock/well. Each compound is run in triplicate plates.

Cell plating: Cells were counted using Vi-Cell XR, then cells were spun down and resuspended in growth media to 200,000 cells/ml. Cells were added to plates containing compound. 10,000 cells/well in 50 ul volume using a Biomek liquid handler. Plates were briefly mixed on a shaker for 1 minute at 2500 rpm then placed in a 37° C., 5% CO₂ incubator overnight for 18-20 hrs.

Measuring luciferase activity: ONE-glo reagent was brought to room temperature, and 25 μL of ONE-glo assay reagent was added to each well using Biomek liquid handler. The plates were incubated for 10 minutes at room temperature, then luminescence was measured on an Envision plate reader.

Results of the Cellular TF1 STAT3 and STAT5 assay are presented in Table 1. Compounds having an IC₅₀ greater than 10 pM, but less than or equal to 10 nM are represented as “A”; compounds having an IC₅₀ greater than 10 nM but less than or equal to 50 nM are represented as “B”; compounds having an IC₅₀ greater than 50 nM but less than or equal to 500 nM are represented as “C”; compounds having an IC₅₀ greater than 500 nM but less than or equal to 5 UM are represented as “D”; and compounds having an IC₅₀ greater than 5 μM but less than or equal to 100 μM are represented as “E”.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A compound of formula I′:
Cy^{A}-L¹-Cy^{B} I′
or a pharmaceutically acceptable salt thereof, wherein: 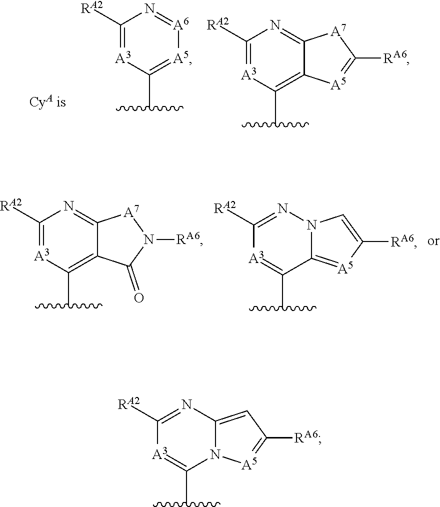
L¹ is —NH—; or
Cy^{A}-L¹- taken together are 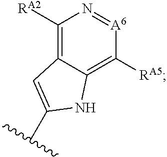
Cy^{B} is 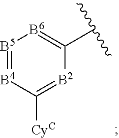
R^{A2} is hydrogen, or —NHR^{A2A}
A³ is N, CH, or C(R^{A3});
A⁵ is N, CH, or C(R^{A5});
A⁶ is N or CH;
A⁷ is NH, S, or CH₂;
B² is N, CH or C(R^{B2});
B⁴ is N, CH or C(R^{B4});
B⁵ is N, CH or C(R^{B5});
B⁶ is N or CH;
Cy^{C} is phenyl, or a 5-6 membered monocyclic heteroaryl ring having 1-4 heteroatoms independently selected from nitrogen, oxygen, and sulfur; wherein Cy^{C} is substituted by R^{C1} and 0-4 instances of R^{C2};
R^{C1} is 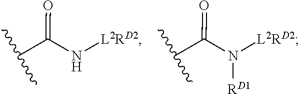
a 3-7 membered saturated or partially unsaturated heterocyclic ring having 1-4 heteroatoms independently selected from nitrogen, oxygen, or sulfur; or a 3-7 membered saturated or partially unsaturated carbocyclic ring; wherein said ring is substituted by -L²R^{D2} and 0-4 instances of R^{CA},
L² is —CH₂—, —CH(R^{L2})—, —C(R^{L2})₂—, —C(O)—, —O—, or —N(R^{L2})—;
each instance of R^{A2A}, R^{A3}, R^{A5}, R^{A6}, R^{B2}, R^{B4}, R^{B5}, R^{C2}, R^{CA}, R^{D1}, and R^{L2} is independently R^{A} or R^{B}, and is substituted by 0-4 instances of R^{C};
R^{D2} is R^{B}, substituted by 0-4 instances of R^{D2A};
each instance of R^{D2A} is independently R^{C}; or an instance of R^{D2A} and R^{D1} are taken together with their intervening atoms to form a saturated or partially unsaturated 3-7 membered ring fused to R^{D2};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; and
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are optionally taken together with their intervening atoms to form an optionally substituted 4-7 membered saturated, partially unsaturated, or heteroaryl ring having 0-3 heteroatoms, in addition to the nitrogen, independently selected from nitrogen, oxygen, and sulfur.

2. The compound of claim 1, wherein Cy^{A} is

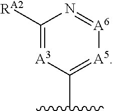

3. The compound of claim 2, wherein R^{A2} is —NHR^{A2A}.

4. The compound of claim 3, wherein R^{A2A} is 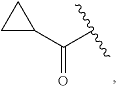 [SMILES: *C(=O)C1CC1]
substituted by 0-4 instances of R^{C}.

5. The compound of any one of claims 1-4, wherein A⁶ is N.

6. The compound of any one of claims 1-5, wherein A⁵ is C(R^{A5}).

7. The compound of any one of claims 1-6, wherein R^{A5} is —C(O)NH₂ or —C(O)NHMe.

8. The compound of any one of claims 1-7, wherein A³ is CH.

9. The compound of any one of claims 1-8, wherein L¹ is —NH—.

10. The compound of any one of claims 1-9, wherein each of B², B⁴, B⁵, and B⁶ is independently CH or C(R^{B2}).

11. The compound of any one of claims 1-10, wherein Cy^{C} is phenyl or a nitrogen-containing 5-6 membered monocyclic heteroaryl substituted by 0-4 instances R^{C2}.

12. The compound of any one of claims 1-11, wherein R^{C1} is 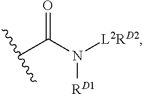
a nitrogen-containing 3-7 membered saturated or partially unsaturated heterocyclic ring, or a 3-7 membered saturated or partially unsaturated carbocyclic ring, each of which is substituted with 0-4 R^{CA}.

13. The compound of any one of claims 1-12, wherein L² is —CH₂— or —CH(R^{L2})—.

14. The compound of any one of claims 1-13, wherein R^{B} is a C₁₋₆ aliphatic chain; phenyl; a 5-6 membered nitrogen-containing monocyclic heteroaryl ring; an 8-10 membered nitrogen-containing bicyclic heteroaryl ring; or a 3-7 membered saturated or partially unsaturated monocyclic carbocyclic ring.

15. The compound of any one of claims 1-14, wherein the compound is a compound of formula (II-a): 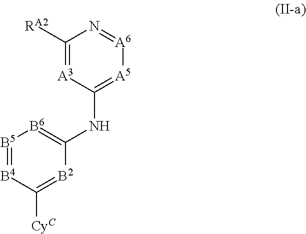
or a pharmaceutically acceptable salt thereof, wherein each of R^{A2}, A³, A⁵, A⁶, B², B⁴, B⁵, B⁶, and Cy^{C} are defined as for claim 1.

16. The compound of any one of claims 1-15, wherein the compound is a compound of formula (III-a): 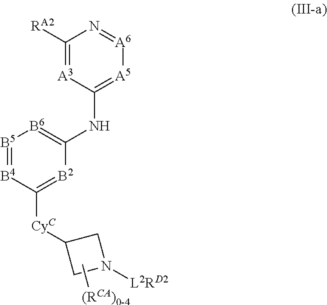
or a pharmaceutically acceptable salt thereof, wherein each of R^{A2}, A³, A⁵, A⁶, B², B⁴, B⁵, B⁶, Cy^{C}, L²R^{D2}, and R^{CA} are defined as for claim 1.

17. The compound of any one of claims 1-16, wherein the compound is a compound of formula (IV-a): 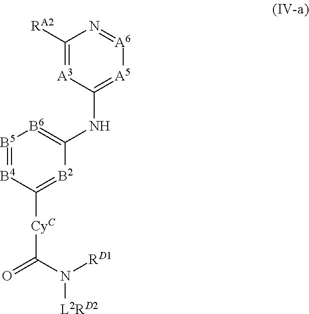
or a pharmaceutically acceptable salt thereof, wherein each of R^{A2}, A³, A⁵, A⁶, B², B⁴, B⁵, B⁶, Cy^{C}, R^{D1}, L²R^{D2}, and R^{CA} are defined as for claim 1.

18. The compound of any one of claims 1-17, wherein the compound is a compound shown in Table 1 or a pharmaceutically acceptable salt thereof.

19. A pharmaceutical composition comprising a compound of any one of claims 1-18, or a pharmaceutically acceptable salt thereof, and a pharmaceutically acceptable carrier, adjuvant, or diluent.

20. A method of inhibiting a JAK2 enzyme in a sample, e.g., in vivo or in vitro, by contacting a JAK2 enzyme with a compound of any one of claims 1-18, or a pharmaceutically acceptable salt thereof, or a composition of claim 19.

21. The method of claim 20, wherein the inhibiting of a JAK2 enzyme comprises reducing the activity of the JAK2 enzyme by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

22. The method of claim 20, wherein the inhibiting of a JAK2 enzyme comprises reducing the activity of the JAK2 enzyme by at least 1-fold, 1.5-fold, 2-fold, 3-fold, 5-fold, 10-fold, 20-fold, 30-fold, 50-fold, 100-fold, or more, e.g., relative to a reference standard.

23. A method of treating a JAK2-mediated disorder in a patient in need thereof, comprising administering to the patient a compound of any one of claims 1-18 or composition of claim 19.

24. The method of claim 23, wherein the JAK2-mediated disorder is a disorder mediated by a JAK2 containing a V617F mutation.

25. The method of any one of claims 23-24, wherein the JAK2-mediated disorder is a proliferative disease.

26. The method of any one of claims 23-25, wherein the proliferative disease displays overexpression or amplification of JAK2, or somatic mutation of JAK2.

27. The method of any one of claims 23-26, wherein the JAK2-mediated disorder is myelofibrosis (MF), polycythemia Vera (PV), essential thrombocythemia (ET), acute megakaryocytic leukemia, T-cell acute lymphoblastic leukemia (T-ALL), B-cell acute lymphoblastic leukemia (B-ALL), acute myeloid leukemia (AML), Chronic Myelomonocytic Leukemia (CMML), T-cell large granular lymphocytic leukemia (T-LGL), T-cell prolymphocytic leukemia (T-PLL), or graft versus host disease (GVHD).

28. The method of any one of claims 23-27, wherein the method comprises the steps of:
(i) identifying a subject in need of such treatment;
(ii) providing a disclosed compound, or a pharmaceutically acceptable salt thereof; and
(iii) administering said provided compound in a therapeutically effective amount to treat, suppress and/or prevent the disease state or condition in a subject in need of such treatment.

29. A compound or pharmaceutically acceptable salt, solvate, stereoisomer, or tautomer thereof, according to any one of claims 1-18 or a composition of claim 19, for use as a medicament.

30. A compound or pharmaceutically acceptable salt, solvate, stereoisomer, or tautomer thereof, according to any one of claims 1-18 or a composition of claim 19 for use in the treatment of a JAK2-mediated disorder.
